(19) United States
(12) Patent Application Publication
(10) Pub. No.: US 2024/0309408 A1
(43) Pub. Date: Sep. 19, 2024

(54) Methods of Use of Oligosaccharide Compositions for Modulating Microbiota and their Metabolic Products, and as Therapeutics for Health Applications

(71) Applicant: BCD BIOSCIENCE, INC., Davis, CA (US)

(72) Inventors: Matthew Joseph AMICUCCI, Davis, CA (US); Angela Maria MARCOBAL-BARRANCO, Davis, CA (US); Steven Michael WATKINS, Davis, CA (US); Maria Ximena MALDONADO GÓMEZ, Davis, CA (US); Nithya KRISHNAKUMAR, Davis, CA (US); Cory Glen VIERRA, Davis, CA (US); Alexandria Marie Salazar CONNER, Davis, CA (US); Riley Anne DREXLER, Davis, CA (US); Yiyun LIU, Davis, CA (US); Jennifer Elizabeth NILL, Davis, CA (US); Bruce Robert MCCONNELL, Davis, CA (US); James Patrick CERNEY, Davis, CA (US); Megan LEE, Davis, CA (US)

(73) Assignee: BCD BIOSCIENCE, INC., Davis, CA (US)

(21) Appl. No.: 18/555,115

(22) PCT Filed: May 12, 2022
(86) PCT No.: PCT/US2022/029065

§ 371 Date: Oct. 12, 2023

Related U.S. Application Data
(60) Provisional application No. 63/188,178, filed on May 13, 2021.
(60) Provisional application No. 63/188,192, filed on May 13, 2021.
(60) Provisional application No. 63/188,239, filed on May 13, 2021.
(60) Provisional application No. 63/188,386, filed on May 13, 2021.
(60) Provisional application No. 63/188,392, filed on May 13, 2021.
(60) Provisional application No. 63/188,395, filed on May 13, 2021.
(60) Provisional application No. 63/188,402, filed on May 13, 2021.
(60) Provisional application No. 63/188,411, filed on May 13, 2021.
(60) Provisional application No. 63/253,864, filed on Oct. 8, 2021.

(51) Int. Cl. C12P 7/6409; C12N 1/20; C12P 7/52; C12P 17/10; C12R 1/145; C12R 1/25

(52) U.S. Cl. CPC C12P 7/6409; C12N 1/205; C12P 7/52; C12P 17/10; C12R 2001/145; C12R 2001/25

(57) ABSTRACT

Provided are oligosaccharides, compositions comprising oligosaccharides, formulations thereof, methods for modulating microbiota and their metabolic products, and methods for the use of same as therapeutics for treating, preventing, or improving conditions associated with various health conditions, including gastrointestinal health, cardiovascular health, renal system health, central nervous system health, immune system health, and urogenital health.

### CROSS-REFERENCE TO RELATED APPLICATIONS

This application claims the benefit of U.S. Provisional Patent Application No. 63/188,239, filed May 13, 2021, U.S. Provisional Patent Application No. 63/188,178, filed May 13, 2021, U.S. Provisional Patent Application No. 63/188,386, filed May 13, 2021, U.S. Provisional Patent Application No. 63/188,192, filed May 13, 2021, U.S. Provisional Patent Application No. 63/188,392, filed May 13, 2021, U.S. Provisional Patent Application No. 63/188,395, filed May 13, 2021, U.S. Provisional Patent Application No. 63/188,402, filed May 13, 2021, U.S. Provisional Patent Application No. 63/188,411, filed May 13, 2021, and U.S. Provisional Patent Application No. 63/253,864, filed Oct. 8, 2021, each of which is incorporated herein by reference in its entirety for all purposes.

### FIELD

The disclosure herein generally relates to oligosaccharides, compositions comprising oligosaccharides, methods to obtain oligosaccharides and oligosaccharide compositions, methods for modulating microbiota and their metabolic products using oligosaccharides or oligosaccharide compositions, and methods for use of oligosaccharides or oligosaccharides as therapeutics for health applications including for gastrointestinal health, cardiovascular health, renal system health, nervous system health, immune system health, and urogenital health.

### BACKGROUND

Chronic medical conditions or diseases are conditions that endure for extended periods and require ongoing medical attention or limit activities of daily living, or both. In some cases, the symptoms may go through phases of flare-ups and relapses while in other cases, the symptoms remain consistently. Chronic diseases are extremely prevalent and are a major contributor to impaired quality of life and health economic burdens. For example, chronic diseases such as heart disease, cancer, and diabetes are the leading causes of death and disability in the United States and are the leading drivers of the country's $3.8 trillion in annual health care costs.

Many chronic diseases are caused by poor life-style choices such as tobacco use, poor nutrition, lack of physical activity, and excessive alcohol use. In other instances, the causes may be attributed to genes or infections. However, it is now becoming clear that many chronic diseases are associated with disruptions or dysfunctionality in the intestinal microbiota. The intestinal microbiota consists of a wide variety of microorganisms, primarily bacteria, viruses, and fungi, that live in the intestine. Generally, the intestinal microbiota forms a symbiotic relationship with the host to the extent that it has been called an essential organ.

The intestinal microbiota has a crucial role in human health through several mechanisms. First, the intestinal microbiota contains far more versatile metabolic genes than the human genome, and therefore provides humans with unique and specific enzymes and biochemical pathways to metabolize foods. For example, the intestinal microbiota has the potential to increase energy extraction from food, increase nutrient harvest, synthesize essential nutrients, and alter appetite signaling. Secondly, the human microbiota provides a defense against infection by protecting the host through competitive exclusion and the production of antimicrobial substances. Thirdly, the microbiota is essential in the development of the intestinal mucosa and immune system of the host. For example, germ-free animals have abnormal immune cell types, deficits in local and systemic lymphoid structures, poorly formed spleens and lymph nodes, and perturbed cytokine levels. The intestinal microbiota is primarily involved in promoting the maturation of immune cells and the normal development of immune functions.

The intestinal microbiota, once disturbed or rendered dysfunctional, also has a role in the development of many chronic diseases.

The intestinal microbiota appears to play a role in all the chronic conditions described above and hence appropriately modulating the microbiota is an attractive potential approach. However, the intestinal microbiota is a complex and very dynamic microbial ecosystem. Selective stimulation of specific intestinal bacteria to promote their growth and metabolic activity is difficult. Antibiotics are able to modulate the microbiota but are not selective and cannot be used over the longer term. The administration of live, beneficial microorganisms (probiotics) is a potential approach. However, the survival and persistence of ingested probiotics is key to effectiveness and many strains have poor persistence. Also, many potential probiotics are extremely difficult to cultivate. A further approach has been the use of oligosaccharides which can be metabolized by specific endemic microorganisms.

Oligosaccharides are short chains of carbohydrates that have been shown to have a variety of functions (e.g., bioactive functions, etc.) that are influenced by a number of structural attributes such as stereochemistry, branching, degree of polymerization, monosaccharide composition, and glycosidic bond positions (Amicucci, Nandita et al. 2019). Oligosaccharides from human milk (HMOs), for example, promote the growth of certain microbes that are nascent to the infant gut, while also modulating the immune system, reducing instances of diarrhea, and protecting the host from pathogen adhesion (Morrow, Ruiz-Palacios et al. 2004, LoCascio, Ninonuevo et al. 2007, Smilowitz, Lebrilla et al. 2014).

However, oligosaccharides are not universally utilized by all microbiota, and even the same type of oligosaccharide, depending on chain length and structure, can have different effects. For example, one study reported that, while oligofructose was utilized by certain Lactobacillus species, the oligofructose did not increase the proportion of lactobacilli (a favorable urogenital organism) and actually stimulated growth of C. albican (an unfavorable pathogen) (Collins et ail. 2018). On the other hand, a different study found that vaginal Lactobacillus isolates were able to utilize shorter chain oligofructose, whereas C. albicans did not (Rosseau et al. 2005).

While oligosaccharides show promise for modulating certain microbiota and their associated metabolic intermediates and products, the ability to access testable oligosaccharides has been limited by the few available methods of production. For example, biological synthesis is currently the primary tool for producing oligosaccharides at scale, including human milk oligosaccharides (Merighi, McCoy et al. 2016, Yu, Liu et al. 2018). However, little work has been done to expand the availability of oligosaccharides beyond HMOs or other common oligosaccharides, such as galactooligosaccharides (GOS) and fructooligosaccharides (FOS) (Gosling, Stevens et al. 2010, Dominguez, Rodrigues et al. 2014). Chemical methods for the depolymerization of polysaccharides, while known in the art, are not routinely employed, but show promise to provide a more robust and broader path to polysaccharide depolymerization to produce oligosaccharides. For example, a method for polysaccharide depolymerization using Fenton's chemistry followed by cleavage using a strong-Arrhenius base (Na⁺OH⁻, K⁺OH⁻ or Ca²⁺⁽(OH⁻)₂) has recently been described (Amicucci, Park et al. 2018, Amicucci, Nandita et al. 2020). Methods based on Fenton chemistry have also been created that employ volatile nitrogen-based bases that can be evaporated off and also actively quench hydrogen peroxide (see WO 2021/097138).

While such methods in some cases provide access to unique oligosaccharides and oligosaccharide compositions, depending on the depolymerization conditions and source of polysaccharide, it is unknown whether a given oligosaccharide or oligosaccharide composition can be utilized by a given microorganism or even if specific oligosaccharide structures can modulate communities of bacteria and metabolites in various microbiomes, including the gut, skin, urogenital, respiratory, oral, and so forth.

Thus, there is a need in the art for oligosaccharides that can modulate microbiomes and/or their metabolic products in targeted directions and associated methods of such modulation. In addition, there remains a need for effective methods for the prophylaxis and/or treatment of chronic medical conditions which are associated with intestinal microbiota.

### SUMMARY

Provided are methods for modifying microbial communities and the associated bioactive metabolites in vitro and/or in the gastrointestinal tract of a subject by applying one or more oligosaccharide compositions of varying structures. Depending on the oligosaccharide structures, microbial community structure (e.g., microbial abundance levels and composition) and metabolism respond in different ways. Also provided are methods of treating diseases, conditions, disorders, and/or indications relating to gastrointestinal health, gut brain axis, cardiovascular disease, and/or metabolic disorders. Also provided are novel oligosaccharide compositions and their structural features. Generally the oligosaccharide compositions are, but need not be, derived from natural products.

A method for modulating microbiota to produce at least one short chain fatty acid and/or to increase an abundance of the microbiota, the method comprising:
contacting the microbiota with a formulation comprising an oligosaccharide composition;
wherein the method modulates the microbiota to produce the at least one short chain fatty acid and/or increase the abundance of the microbiota; and
wherein the oligosaccharide composition comprises:
   (1) at least one first feature comprising:
      a sum of glucose, galactose, and mannose subunits in an amount of at least 40 wt. %, based on total weight of saccharide subunits; or
   (2) at least one second feature comprising:
      a sum of rhamnose, galacturonic acid, arabinose, fucose, and mannose subunits in an amount of at least 3 wt. %, based on total weight of saccharide subunits;
      a sum of xylose subunits in an amount of at least 33 wt. %, based on total weight of saccharide subunits;
      a sum of mannose subunits in an amount of at least 50 wt. %, based on total weight of saccharide subunits; and a sum of galactose subunits in an amount of less than 20 wt. %, based on total weight of saccharide subunits;
      a weight ratio of mannose subunits to glucose subunits between 2:1 and 4:1; and wherein at least 70 wt. % of the non-terminal mannose subunits have at least one 2-linkage, and wherein at least 70 wt. % of the non-terminal glucose subunits have at least one 3-linkage, at least one 4-linkage, and/or at least one 6-linkage; or
      any combination thereof; or
   (3) at least one third feature comprising:
      a sum of glucose, galactose, mannose subunits in an amount of at least 50 wt. %, based on total weight of saccharide subunits;
      a sum of glucose subunits in an amount of at least 20 wt. %, based on total weight of saccharide subunits; and a sum of xylose and arabinose subunits in an amount of at least 33 wt. %, based on total weight of saccharide subunits; or
      any combination thereof.

A method for modulating microbiota to: produce at least one indole derivative, metabolize bile acid and/or bile salt, increase an abundance of the microbiota, or any combination thereof; the method comprising:
contacting the microbiota with a formulation comprising an oligosaccharide composition;
wherein the method modulates the microbiota to: produce the at least one indole derivative, metabolize the bile acids and/or bile salts, increase the abundance of the microbiota, or any combination thereof; and
wherein the oligosaccharide composition comprises:
   a sum of galactose subunits in an amount of 3 wt. % to 60 wt. % galactose, based on total weight of saccharide subunits; and a sum of arabinose subunits in an amount of 20 wt. % to 90 wt. %, based on total weight of saccharide subunits;
   non-terminal glucose subunits, wherein (1) 20 wt. % to 100 wt. % of the non-terminal glucose subunits have at least one 3-linkage, based on total weight of non-terminal saccharide subunits; and/or (2) 33 wt. % to 80 wt. % of the non-terminal glucose subunits have at least one 4-linkage, based on total weight of non-terminal saccharide subunits;
   a sum of mannose subunits in an amount of 33 wt. % to 70 wt. %, based on total weight of saccharide subunits; and a sum of glucose subunits in an amount of 18 wt. % to 45 wt. %, based on total weight of saccharide subunits;
   a sum of xylose subunits in an amount of 33 wt. % to 70 wt. %, based on total weight of saccharide subunits; and a sum of arabinose subunits in an amount of 18 wt. % to 45 wt. %, based on total weight of saccharide subunits; or
   any combination thereof.

An oligosaccharide composition, comprising a sum of galactose and arabinose subunits in an amount of at least 33 wt. %, based on total weight of saccharide subunits.

An oligosaccharide composition, comprising a sum of 3-linked and 4-linked glucose subunits in an amount of between 7-50 wt. %, based on total weight of saccharide subunits.

An oligosaccharide composition, comprising at least 35 wt. % fucose subunits, based on total weight of saccharide subunits.

An oligosaccharide composition, comprising at least 25 wt. % rhamnose subunits, based on total weight of saccharide subunits.

A formulation comprising an oligosaccharide composition.

A method for treating or preventing a disease, condition, disorder, and/or indication in a subject in need thereof, the method comprising:
administering to the subject the formulation comprising an oligosaccharide composition;
wherein the method treats or prevents the disease, condition, disorder, and/or indication in the subject.

An oligosaccharide composition that is or comprises CLX101, CLX101C, CLX102, CLX103, CLX105, CLX107, CLX108, CLX109, CLX110, CLX111, CLX112, CLX113, CLX114, CLX115, CLX115AL, CLX115-FC, CLX115A, CLX116, CLX117, CLX118, CLX119, CLX121, CLX122, CLX122DS, CLX122DSF, CLX123, CLX124, CLX125, CLX126, CLX127, CLX128, CLX129, CLX130, CLX131, CLX132, CLX133, or any combination thereof.

Without wishing to be bound by any particular theory, there may be discussion herein of beliefs or understandings of underlying principles relating to the devices and methods disclosed herein. It is recognized that regardless of the ultimate correctness of any mechanistic explanation or hypothesis, an embodiment or aspect of the invention can nonetheless be operative and useful.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 shows, by way of non-limiting examples, comparison of the total production of oligosaccharides from locust bean gum by different cleaving reagents and temperatures.

FIG. 2 shows, by way of non-limiting examples, locust bean gum oligosaccharide profiles of different cleaving reagents reacted at 45° C.

FIG. 3 shows, by way of non-limiting examples, residual hydrogen peroxide after incubation with three exemplary cleavage reagents at 27° C. for one hour.

FIG. 4 shows, by way of non-limiting examples, hydrogen peroxide concentration and pH after incubation with ammonium bicarbonate for one hour at varying temperatures.

FIGS. 5A and 5B show, by way of non-limiting examples, liquid chromatography-mass spectrum of two spent distiller's grain fractions.

FIG. 6 shows, HPLC/Q-TOF chromatogram of oligosaccharides generated from amylopectin. Oligosaccharides are generated from the base cleavage step using ammonium hydroxide or sodium hydroxide.

FIG. 7 shows, monosaccharide composition of oligosaccharides generated from amylopectin. Oligosaccharides are generated from the base cleavage step using ammonium hydroxide or sodium hydroxide. Monosaccharide abundance is normalized to that of the control.

FIG. 8 shows, bacterial growth of oligosaccharides generated from amylopectin. Oligosaccharides are generated from the base cleavage step using ammonium hydroxide or sodium hydroxide.

FIG. 9 shows, oligosaccharide analysis of amylopectin oligosaccharides generated from the base cleavage step using different strong Arrhenius and nitrogen-containing bases.

FIG. 10 shows, monosaccharide composition of locust bean gum polysaccharides and locust bean gum oligosaccharides.

FIG. 11 shows, HPLC/Q-TOF chromatogram showing COG-derived locust bean gum oligosaccharides.

FIG. 12 shows, the comparison of corn fiber oligosaccharide production using differing catalysts and conditions.

FIG. 13A-B shows, 1H-13C HSQC NMR spectra of COG-derived oligosaccharides.

FIG. 14A-B shows, annotated Extracted Ion Chromatograms with the most abundant oligosaccharides labeled.

FIG. 15 shows, annotated linkage analysis chromatogram of corn fiber.

FIG. 16: CLX115-dependent increases in Propionate, L-Malate, Succinate, and Glycerate, and a CLX115-dependent spike in Butyrate, Beta-hydroxybutyrate and Lactate, and a strong depletion of murine bile acids.

FIG. 17A-C: Comparison of Lactic Acid, Succinic Acid, and Acetic Acid production in Bif. longum subsp. longum.

FIG. 18A-B: Comparison of Lactic Acid and Succinic Acid production in Bif. longum subsp. infantis.

FIG. 19A-B: Comparison of Succinic Acid and Lactic Acid production in Bif pseudocatenulatum.

FIG. 20A-B: Comparison of Lactic Acid and Succinic Acid production in L. crispatus.

FIG. 21: Comparison of Lactic Acid production in L. rhamnosus.

FIG. 22: Comparison of Butyric Acid production in Cl. butyricum.

FIG. 23: Relative abundance of Bifidobacterium, Blautia, Roseburia, Cl. butyricum and Ruminococcus genus after 20 hours in vitro fecal fermentation in the present of different oligosaccharides.

FIG. 24: Comparisons of short chain fatty acid and organic acid production in oligosaccharides over 3 timepoints: 6, 10 and 20-hour.

FIGS. 25A-25B: Decreased utilization of (FIG. 25A) choline and (FIG. 25B) methionine.

FIGS. 26A-26D: (FIG. 26A) Increased Nicotinic Acid, (FIG. 26B) Pantothenic Acid production in comparison to a 0-hour timepoint, (FIG. 26C) Isoleucine production comparison between 6 and 10 hours, and (FIG. 26D) Valine production between 0 and 20 hours.

FIGS. 27A-27C: (FIG. 27A) Increase in Gamma-aminobutyric acid, (FIG. 27B) Glutamic Acid and (FIG. 27C) Ornithine production.

FIG. 28A-D: (FIG. 28A) Decrease over time in Histamine, (FIG. 28B) Cadverine and (FIG. 28C) Putrescine production, and (FIG. 28D) Percent difference in putrescine levels.

FIG. 29A: Increased Propionic Acid and (FIG. 29B) Lactic Acid production in CLX111 and CLX114 as compared to the corresponding polysaccharides.

FIG. 30: Increased Ornithine levels in CLX111 and CLX112 as compared to the corresponding polysaccharides.

FIG. 31: Colony forming units per mL (CFU/mL) of Lactobacillus rhamnosus present after anaerobic incubation at 37° C. for 24 hours on glucose, CLX112, and CLX112 polysaccharide (PS) as the sole carbon source and on a no carbohydrate control (Basal) (n=3).

FIG. 32A: CFU/mL of Lactobacillus rhamnosus present in a complex community after 24 hours of in-vitro fecal fermantation both with and without CLX112 as the main carbon source (0.6%). L. rhamnosus was spiked into the fecal fermentation at 0.01% of the total bacterial load (t=0).

FIG. 32B: Enrichment of L. rhamnosus after anaerobic incubation supplemented with various ratios of CLX112 and FOS.

FIG. 33: The free monosaccharide composition analysis of CLX115 is shown with various catalysts and metal concentrations.

FIG. 34: The oligosaccharide DP analysis of CLX115 is shown with various catalysts and metal concentrations.

FIG. 35: The free monosaccharide composition analysis of CLX115 is shown with selective precipitation through various ethanol concentrations.

FIG. 36: The oligosaccharide DP analysis of CLX115 is shown with selective precipitation through various ethanol concentrations.

FIG. 37: The SCFA compositional analysis of CLX115 post fecal community modification is shown with selective precipitation through various ethanol concentrations.

FIG. 38: The oligosaccharide analysis chromatograms of CLX101, CLX110, CLX112 and CLX115 are shown with DP based labels.

FIG. 39: The SCFA compositional analysis of CLX101, CLX110, CLX112 and CLX115 is shown post fecal community modification.

FIGS. 40A-40F show ¹H-13C HSQC NMR spectra of CLX107, CLX115A, CLX116, CLX117, CLX118, and CLX119 recorded on a Brucker 600 MHz NMR spectrometer in DMSO-d₆ with 0.03% TMS.

FIGS. 41A-41F show ¹H-13C HSQC NMR spectra of CLX121-CLX126 recorded on a Brucker 600 MHz NMR spectrometer in DMSO-d₆ with 0.03% TMS.

FIGS. 42A-42P show ¹H-13C HSQC NMR spectra of CLX127-CLX132 recorded on a Brucker 600 MHz NMR spectrometer in DMSO-d₆ with 0.03% TMS.

FIG. 43 is a bar graph showing growth of Bifidobacteria strains using CLX122 as sole carbon source (2%), measured by absorbance at 600 nm (n=3). Negative control is minimum media without carbohydrate.

FIG. 44 is a graph showing changes in relative abundance of Bifidobacteria in fecal fermentations with a fecal pool sample in the presence of CLX122, CLX127, CLX126 and CLX128 versus untreated control.

FIG. 45 is a graph showing changes in relative abundance of Bacteroides in fecal fermentations with a fecal pool sample in the presence of CLX122, CLX127, CLX126 and CLX128 versus untreated control.

FIG. 46 is a graph showing changes in relative abundance of Clostridium butyricum in fecal fermentations with a fecal pool sample in the presence of CLX122, CLX127, CLX126 and CLX128 versus untreated control.

FIG. 47 is a graph showing changes in relative abundance of Roseburia and Blautia in fecal fermentations with a fecal pool sample in the presence of CLX122, CLX127, CLX126 and CLX128 versus untreated control.

FIG. 48 is a graph showing changes in absolute abundance of butyric acid and lactic acid in fecal fermentations with a fecal pool sample in the presence of CLX122, CLX127, CLX126 and CLX 128.

FIG. 49 is a bar graph showing changes in absolute abundance of monosaccharides in fecal fermentation supernatant in the presence of CLX122 at 0 hrs and 20 hrs of incubation. Background sugars have been subtracted to ease interpretation.

FIG. 50A Changes in relative abundance of Bifidobacteria in 15 fecal fermentations from unique donors, in the presence of CLX122 (dark circles) versus untreated control (light circles).

FIG. 50B: Quantitiave changes of Bifidobacteria in 15 fecal fermentations from unique donors, in the presence of CLX122 (dark circles) versus untreated control (light circles).

FIG. 51 is a graph showing changes in short chain fatty acid composition of 15 fecal fermentations from unique donors in the presence of CLX 122 (dark circles) versus untreated control (light circles).

FIG. 52 is a graph showing Cl. butyricum growth was supported by CLX126 and CLX128.

FIG. 53 is a graph showing Bif. pseudocatenatum growth was supported by CLX122, CLX126, CLX127 and CLX128. Bif. longum subsp. infantis growth was supported by CLX128, and Bif. longum subsp. longum growth was supported by CLX128.

FIG. 54 Changes in relative abundance of Cl. butyricum in 15 fecal fermentations from unique donors, in the presence of CLX115 versus untreated control.

FIG. 55 Quantitiave changes of Cl. butyricum in 15 fecal fermentations from unique donors, in the presence of CLX115 (dark circles) versus untreated control (light circles).

FIG. 56 is a graph showing changes in short chain fatty acid composition of 15 fecal fermentations from unique donors in the presence of CLX115 (dark circles) versus untreated control (light circles).

FIG. 57A Butyrate concentrations of CLX pools after 20 hours of fermentation.

FIG. 57B is a flow chart or decision tree for determining whether an oligosaccharide composition may produce high levels of butyrate.

FIG. 58A Propionate concentrations of CLX pools after 20 hours of fermentation.

FIG. 58B is a flow chart or decision tree for determining whether an oligosaccharide composition may produce high levels of propionate.

FIG. 59A Beta-hydroxybutyrate concentrations of CLX pools after 20 hours of fermentation.

FIG. 59B is a flow chart or decision tree for determining whether an oligosaccharide composition may produce high levels of beta-hydroxybutyrate.

FIG. 60 is a graph showing CLX pools responsible for the production of bioactive indole derivates.

FIG. 61 is a graph showing CLX pools responsible for the conversion of bile acids.

FIG. 62 Experimental schematic for animal experiment assessing the effects of CLX115 treatment on microbial community composition and metabolite profile in a carbohydrate-deficient diet model.

FIG. 63 Measurements of body weight and water consumption during the experiment demonstrate palatability and lack of adverse effects of CLX115 treatment on animal health.

FIG. 64A-B: (FIG. 64A) is a bar graph showing CLX115 does not have a significant effect on multiple measures of alpha diversity of gut microbial communities. (FIG. 64B) is a figure showing CLX115 supplementation reversibly shifts community composition, as depicted in a PCOA plot constructed using Bray Curtis distances. Rings symbols correspond to mice receiving CLX115 and star symbols correspond to the untreated control. Large, light-grey symbols correspond to Day 0 (before supplementation), dark-grey symbols correspond to CLX115 consumption (small: day 6, big: day 8), small, light-grey symbols correspond to last day of washout (day 12).

FIG. 65 is a bar graph showing CLX115 treatment resulting in an enrichment in multiple taxa, including members of the genus Bacteroides and species within the Blautia, Clostridia UCG-014 and Harryflintia genera.

### STATEMENTS REGARDING CHEMICAL COMPOUNDS AND NOMENCLATURE

In general, the terms and phrases used herein have their art-recognized meaning, which can be found by reference to standard texts, journal references and contexts known to those skilled in the art. The following definitions are provided to clarify their specific use in the context of the invention.

As used herein, the terms “about” and “approximately,” when used to modify an amount specified in a numeric value or range, mean that slight variations from a stated value may be used to achieve substantially the same results as the stated value. In circumstances where this definition cannot be applied or is exceedingly difficult to apply, then the terms “about” and “approximately” mean a reasonable deviation from the value known to a skilled person in the art, such as, if “X” were the value, for example, “about X” or “approximately X” would indicate a value from 0.9X to 1.1X, e.g., a value from 0.95X to 1.05X, or a value from 0.98X to 1.02X, or a value from 0.99X to 1.01X. Any reference to “about X” or “approximately X,” where X is a value disclosed herein, specifically indicates at least the values X, 0.9X, 0.91X, 0.92X, 0.93X, 0.94X, 0.95X, 0.96X, 0.97X, 0.98X, 0.99X, L1X, 1.02X, 1.03X, 1.04X, 1.05X, 1.06X, 1.07X, 1.08X, 1.09X, and 1.1X, and values within this range. It is specifically contemplated that, when a given CLX composition or analysis method has “approximately” or “about” the values set forth in a table or spectrum, the definitions of the terms “approximately” or “about” in this paragraph apply to such CLX composition or analysis method.

As used herein, the terms “collectively comprise,” “collectively comprises,” or similar terms, when in reference to one or more oligosaccharides (or any similar context), means that the one or more oligosaccharides as a whole have the indicated composition or property. For example, a given composition can contain two different oligosaccharides (e.g., a first oligosaccharide comprising glucose subunits but not arabinose subunits, and a second oligosaccharide comprising arabinose subunits but not glucose subunits). In this situation, the these two different oligosaccharides (i.e., the “one or more oligosaccharides”) collectively comprise glucose and arabinose subunits. Similarly, the term “collectively” has the same meaning in any similar context (e.g., in reference to a composition, etc.) to indicate that any components that are present collectively have an indicated feature or property (e.g., NMR analysis or linkage types or amounts, etc.).

Any viscosity measurement or property reported herein employs water as the solvent, unless specified otherwise. Viscosity generally is measured according to Example 16.

In some aspects, a composition or compound disclosed herein, such as an oligosaccharide or oligosaccharide composition, is isolated or substantially purified. In an embodiment, an isolated or purified oligosaccharide or oligosaccharide composition is at least partially isolated or substantially purified as would be understood in the art. In some aspects, a substantially purified composition, oligosaccharide or formulation disclosed herein has a chemical purity of 95%, optionally for some applications 99%, optionally for some applications 99.9%, optionally for some applications 99.99%, and optionally for some applications 99.999% pure.

When disclosing numerical values herein, for example, 1, 2, 3, 4, 5, 6, 7, 8, 9, 10, the following sentence typically follows such numerical values: “Each of the foregoing numbers can be preceded by the term ‘about,’ ‘at least,’ ‘at least about,’ ‘less than,’ or ‘less than about,’ and any of the foregoing numbers can be used singly to describe a single point or an open-ended range, or can be used in combination to describe multiple single points or a close-ended range.” This sentence means that each of the aforementioned numbers can be used alone (e.g., 4), can be prefaced with the word “about” (e.g., about 8), prefaced with the phrase “at least about” (e.g., at least about 2), prefaced with the phrase “at least” (e.g., at least 10), prefaced with the phrase “less than” (e.g., less than 1), prefaced with the phrase “less than about” (e.g., less than about 7), or used in any combination with or without any of the prefatory words or phrases to define a range (e.g., 2 to 9, about 1 to 4, at least 3, 8 to about 9, 8 to less than 10, about 1 to about 10, and so on). Moreover, when a range is described as “about X or less,” this phrase is the same as a range that is a combination of “about X” and “less than about X” in the alternative. For example, “about 10 or less” is the same as “about 10, or less than about 10.” Such interchangeable range descriptions are contemplated herein. Other range formats may be disclosed herein, but the difference in formats should not be construed to imply that there is a difference in substance.

When a number of repeat units (e.g., DP), hexose subunits, pentose subunits, carbon atoms of an alkyl group, and so forth, is specified herein, it is intended that the number refers to the exact number of the relevant species, or range thereof, that is specified. In other words, when a number of repeat unit is specified, it is not intended that an oligosaccharide thereof comprises that specified number of repeat units, but rather that the oligosaccharide contains the specified number. For example, if an oligosaccharide is specified to contain 4 to 8 repeat units, an oligosaccharide containing 12 carbon atoms would not qualify nor would an oligosaccharide containing 3 repeat units; rather, only an oligosaccharide that contains 4, 5, 6, 7, or 8 repeat units would qualify. The same concept applies to other similar features, such as number of hexose subunits, pentose subunits, carbon atoms of an alkyl group, and so forth.

As used herein, the term “polysaccharide” refers to a polysaccharide or a material comprising a polysaccharide, in either case wherein at least the polysaccharide component is cleavable by the COG methods disclosed herein. Additionally, as used herein, the term “polysaccharide” refers to any carbohydrate polymer and can also be linked to other non-carbohydrate moieties (e.g., glycoproteins, proteoglycans, glycopeptides, glycolipids, glycoconjugates, glycosides, or any combination thereof). Moreover, as used herein “polysaccharide” refers to a polymer of monosaccharide units of greater than 30 monosaccharide units, and can reach hundreds of thousands of monosaccharides in length. A polysaccharide can be a linear polymer, branched polymer, primarily linear polymer with pendant saccharide monomers, or any combination thereof.

As used herein, the term “peroxide agent” refers to compounds that contain oxygen-oxygen bonds that can produce, natively, with light, temperature, or catalyst (e.g., metals and enzymes), R—O⁻ and/or R—O—O⁻ species, where “R” refers to a hydrogen or carbon group that is attached to the rest of the molecule. In one aspect, a peroxide agent is hydrogen peroxide.

The “degree of polymerization” or “DP” of an oligosaccharide refers to the total number of sugar monomer units that are part of a particular carbohydrate. For example, a tetra galacto-oligosaccharide has a DP of 4, having 3 galactose moieties and one glucose moiety.

As used herein, when the amount of a component is expressed in terms of weight or mole percent, it is intended that the amount is on a dry basis unless otherwise specified. As used herein, “dry basis” means in the absence of water or other solvent. For example, when a composition comprises 10 g of glucose, 40 g of xylose, and 50 g of water, it means the composition comprises 25% (mass % or wt. %) glucose on a dry basis, but the glucose is present in the composition at a concentration of 10% (mass % or wt. %).

A “prebiotic” or “prebiotic nutrient” is generally a non-digestible or partially-digestible (i.e., digestible by the subject/human/animal, and does not include digestion by microbes) food ingredient that beneficially affects a host when ingested by selectively stimulating the growth and/or the activity of one or a limited number of microbes in the gastrointestinal tract, urogenital system, or other portion of the host. As used herein, the term “prebiotic” refers to the above described non-digestible or partially-digestible food ingredients in their non-naturally occurring states, e.g., after purification, chemical or enzymatic synthesis as opposed to, for instance, in whole human milk.

A “probiotic” refers to live microorganisms that when administered in adequate amounts confer a health benefit on the host.

As used herein, a “peeling reaction” or “peeling” as applied to the disclosed methods refers to the sequential alkaline degradation of carbohydrates through a mechanism that releases monomeric units from the reducing end of the polymer.

As used herein, a “cleavage agent” or “cleavage reagent” as applied to the disclosed methods preferably refers to a single or collection of non-Arrhenius and/or weak-Arrhenius bases used to cleave polysaccharides after hydroperoxyl oxidation thereof. In certain aspects, a cleavage agent or cleavage reagent breaks glycosidic bonds in the polysaccharide, which bonds may be present between any two saccharides of the polysaccharide. In the methods described herein, the cleavage reagent (cleavage initiator) may also be, and preferably is a peroxide-quenching reagent, and in either case may be used in combination with an additional compatible peroxide-quenching agent that may or may not also be a cleavage agent. In some aspects, a cleavage reagent may be an enzyme. In some aspects, the cleavage reagent enzyme may be a glycosyl hydrolase, a lytic polysaccharide monooxygenase, a glycosyl transferase, transglycosidase, polysaccharide lyase, carbohydrate binding module, glycoysl transferase, carbohydrate esterase, a cocktail containing two or more of the forementioned enzymes, or any enzyme that is carbohydrate active. In some aspects, a cleavage reagent may be a solid-phase acid catalyst or a solid-phase base catalyst.

As used herein, a “base” refers to a compound or collection of compounds that can accept hydrogen ions from the peroxyl oxidized carbohydrate, water, or non-aqueous solvent. The term “base” can include Lewis bases, non-Arrhenius bases, weak-Arrhenius bases, other molecules that produce through their decomposition hydroxide ions, Lewis bases, non-Arrhenius bases, or weak-Arrhenius bases, or other compounds that can accept hydrogen ions from the hydroperoxyl oxidized carbohydrate. As used herein, unless otherwise specified, a “base” explicitly does not refer to a strong-Arrhenius base (e.g., Na⁺OH⁻, K⁺OH⁻, or Ca⁺²(OH⁻)₂).

As used herein, a “ammonium bicarbonate” as applied to the disclosed methods refers to solid ammonium bicarbonate, and/or an aqueous solution containing: ammonium and bicarbonate; ammonium, OH—, and CO₂; ammonia, H₂O, and CO₂; or any of the preceding and their equilibrium products.

As used herein, “ammonium hydroxide” as applied to the disclosed methods refers to: aqueous ammonium hydroxide, and/or a solution containing: ammonia and H₂O; ammonium and OH⁻; ammonia and OH⁻; or any of the preceding and their equilibrium products.

As used herein, a “strong-Arrhenius base” as applied to the disclosed methods refers to a compound that completely dissociates in water to release one or more hydroxide ions into solution. As used herein, a “strong-Arrhenius base” as applied to the disclosed methods refers explicitly to KOH, NaOH, Ba(OH)₂, CsOH, Sr(OH)₂, Ca(OH)₂, LiOH, and RbOH.

As used herein, a “weak-Arrhenius base” as applied to the disclosed methods refers to a compound that incompletely dissociates in water to release one or more hydroxide ions into solution, e.g. ammonium hydroxide, H₂O, etc. As “weak-Arrhenius base” is used herein, there are no compounds which meet both the definition of strong-Arrhenius base and weak-Arrhenius base.

As used herein, a “non-Arrhenius base” as applied to the disclosed methods refers to a compound or atom that can donate electrons (e.g., Lewis Bases), accept protons (e.g., Bronstead-Lowry Bases), or releases hydroxide ions through its decomposition (NH₄HCO₃), but explicitly does not qualify as an Arrhenius base.

As used herein, a “Lewis base” as applied to the disclosed methods refers to a compound or atom that can donate electron pairs (e.g., F⁻, benzene, H⁻, pyridine, acetonitrile, acetone, urea, etc.).

As used herein a “Bronsted-Lowry base” as applied to the disclosed methods refers to a compound or atom that can accept or bond to a hydrogen ion (e.g., methanol, formaldehyde, ammonia, etc.).

As used herein a “Peroxide quenching reagent” as applied to the disclosed methods refers to a compound or atom, which is not a strong-Arrhenius base, that can convert hydrogen peroxide, peroxyl radicals, and hydroperoxyl radicals to a less reactive or non-reactive state (e.g., ammonium hydroxide, ammonium bicarbonate, ammonia, etc.). In certain aspects, a peroxide quenching reagent as defined herein converts hydrogen peroxide as well as radicals produced from hydrogen peroxide to less reactive species (e.g. water). In certain aspects, a peroxide quenching reagent may reduce the hydrogen peroxide concentration to zero, below 5 mg/L, below 10 mg/L, below 25 mg/L, or below 50 mg/L. In certain aspects, a peroxide quenching reagent may form water, hydroxide ions, or oxygen gas. In certain aspects, enzymes may be used to quench peroxide species. In certain aspects, those enzymes may include catalases. In certain aspects, those enzymes can be from animal origin. In certain aspects, those enzymes can be from bovine liver. In certain aspects, the enzymes may be from microbial origin. In certain aspects, the enzyme may be recombinant. In certain aspects, different enzymes may be mixed to quench the peroxide species.

As used herein “nitrogen-based” as applied to the disclosed methods refers to a compound that contains at least one nitrogen atom with four substituent groups that can contain any combination of lone pairs of electrons, hydrogens, or carbon atoms (e.g., ammonia, sodium amide, trimethylamine, diethylamine, N,N-Diisopropylethylamine, urea, pyridine, ammonium hydroxide, ammonium bicarbonate, etc.). Exemplary nitrogen-based, peroxide-quenching, PS-cleavage agents are listed in Table 1. A nitrogen-based reagent may have an unsubstituted or substituted ammonium group and can be present in neutral and/or ionic forms.

As used herein “reaction mixture” refers to a mixture comprising reagents which may react chemically to form products which are distinct from the reagents.

As used herein “treated polysaccharide” refers to a polysaccharide which has been contacted with at least one reagent capable of reacting with the polysaccharides (e.g. an enzyme or a Fenton's reagent).

As used herein “polysaccharide cleavage product” is a product formed from the chemical and/or enzymatic cleavage of a polysaccharide.

As used herein “oligosaccharide” refers to an oligomer of saccharides, in which the DP of the oligomer is between 2 and 50 monosaccharide units, such as between 3-50, 3-30, 3-20, 3-15, 3-10, 3-8, 3-6, or 5-15 monosaccharide units. An oligosaccharide can be linear, branched, primarily linear with pendant saccharide monomers, or any combination thereof. An “oligosaccharide” refers to an individual oligomer chain. As used herein, “oligosaccharide composition” (also termed “oligosaccharide pool” herein) refers to a mixture of two or more oligosaccharides, each of which can be the same or different from one another. Although efforts have been made herein to consistently use the terms “oligosaccharide” and “oligosaccharide composition” according to their preceding definitions, the intended meaning will be clear from context when such terms are used herein.

As used herein, “subunit” means a species that is covalently bonded to or within an oligomer (e.g., oligosaccharide) or polymer (e.g., polysaccharide). Such species generally can include saccharides (e.g., glucose, galactose, mannose, etc.). For example, when an oligosaccharide composition comprises a glucose subunit, it means that the composition comprises a glucose molecule that is bound to or within an oligomer or polymer; as such, a composition that contains only free monomeric glucose would not contain a glucose subunit. Similarly, when an oligosaccharide composition comprises a sum of glucose, galactose, and mannose subunits in an amount of at least 60 wt. % based on total weight of saccharide subunits, this means that the mass of all of the glucose subunits, galactose subunits, and mannose subunits are summed, and the subunits of all saccharides are summed, and then the first sum is divided by the second sum. Additionally, when an oligosaccharide composition comprises non-terminal galactose subunits, and at least 70 wt. % of the non-terminal galactose subunits are specified to have at least one 4-linkage, this feature is calculated by summing the mass of all non-terminal galactose subunits having at least one 4-linkage (and this can include, for example, galactose subunits with 4,6-linkages and 4,3-linkages), and then dividing by the total mass of non-terminal galactose subunits regardless of linkage type. The same concept is applicable to any feature herein where reference to “at least one X-linkage,” in which X is an integer (e.g., such as “a weight ratio of glucose subunits having at least one 4-linkage to glucose subunits having at least one 3-linkage is between 2:1 to 4:1” and other such features). Moreover, in such calculations the actual mass of the subunit is used (i.e., in bound form) rather than the mass of the unit as if it was hydrolyzed (which would add the mass of water). Other features described elsewhere herein can be calculated similarly. These features can be determined with the aid of the various analytical techniques described herein, such as hydrolytic monosaccharide compositional analysis, oligosaccharide analysis, glycosidic linkage analysis, NMR HSQC Analysis, and so forth, as well as other techniques known in the art.

As used herein “Fenton's reagent” refers to a reagent comprising a peroxide agent and a metal. In certain aspects, the peroxide agent is hydrogen peroxide. In certain aspects, the metal is Fe(II), Fe(III), Cu(I), Cu(II), Mn(II), Zn(II), Ni(II), and Co(II), alkaline earth metals Ca(II) and Mg(II), the lanthanide Ce(IV) or any combination thereof.

As used herein the phrase “substantially commensurate with initiation of peroxide-quenching” refers to the relationship between the timing of a cleavage reaction and the timing of a peroxide quenching reaction indicating that the initiation of the cleavage reaction and the initiation of the peroxide quenching reaction occur within a short time duration of each other (e.g. on the order of seconds, or on the order of minutes but not more than one day).

As used herein “specified reaction time” or “reaction time” refers to providing time to allow a reaction to proceed toward an equilibrium state between reagents added and products produced by the reaction of the reagents. In certain aspects, specified reaction time allows sufficient time to reach an equilibrium. In certain other aspects, specified reaction time, while allowing time for the reaction to proceed toward equilibrium, does not provide the time needed to reach equilibrium.

As used herein, the term “synthetic oligosaccharide” refers to an oligosaccharide produced by the depolymerization of one or more polysaccharides. In certain aspects, the term synthetic oligosaccharide refers to compositions of oligosaccharides produced by the methods disclosed herein. The depolymerization to produce synthetic oligosaccharides can alternatively or additionally take place using enzymes, chemical reactions such as Fenton's chemistry, physical processes such as elevated time and temperature, and so forth, or any combination thereof. In some aspects, the term synthetic oligosaccharide refers to oligosaccharides prepared by synthesizing the oligosaccharide from monosaccharides or lower DP oligosaccharides. The term “synthetic oligosaccharide” is used interchangeably herein with “oligosaccharide,” and “composition comprising at least one synthetic oligosaccharide” is used interchangeably herein with “oligosaccharide composition.” No difference in meaning is intended.

As used herein, the term “synthetic composition” means a composition which is artificially prepared and preferably means a composition containing at least one compound that is produced ex vivo chemically and/or biologically, e.g., by means of chemical reaction, enzymatic reaction, recombinantly, or any combination thereof. The synthetic composition typically comprises one or more compounds, including one or more of the oligosaccharides described herein. In some aspects, the oligosaccharides and oligosaccharide compositions can be formulated into a synthetic composition or administered as the oligosaccharide alone. For example, the synthetic composition can be in the form of a nutritional composition or a pharmaceutical composition.

As used herein, the term “heteropolymer polysaccharide” refers to a polysaccharide containing two or more kinds of monosaccharide subunits linked together by the same type of glycosidic bond or different types of glycosidic bonds; heteropolymer polysaccharides also include polysaccharides containing repeating monosaccharide subunits of the same kind linked together by different types of glycosidic bonds. The glycosidic bonds in a heteropolymer polysaccharide may be β1-2 bonds, β1-3 bonds, β1-4 bonds, β1-5, β1-6 bonds, α1-3 bonds, al-4 bonds, β1-5, α1-6 bonds, or a combination thereof. Examples of heteropolymer polysaccharides include, but are not limited to, xyloglucan, lichenan, β-glucan, glucomannan, galactomannan, arabinan, xylan, and arabinoxylan.

As used herein, “short chain fatty acid” includes butyrate, propionate, betahydroxybutyrate, lactate, acetate, or any combination thereof.

As used herein, the term “hydrolytic monosaccharide compositional analysis” refers to the method described in Amicucci, Galermo et al. 2019, with the following modifications, hereby incorporated by reference in its entirety for all purposes. The hydrolysis reaction to produce monosaccharides was performed at the optimized condition of 100° C. for 2 hours. Samples were ran on an Agilent 1290 Infinity II ultra-high performance liquid chromatography (UHPLC) system couple to an Agilent 6490A triple quadrupole (QqQ) mass spectrometer. Separation was carried out on an Agilent InfinityLab Poroshell HPH-C18 column (2.1 mm×50 mm, 1.9 μm particle size) plus a guard column (5 mm) with the same solvent system described in Amicucci, Galermo et al. 2019. With a constant flow rate of 1.2 mL/min, an isocratic gradient of 8.5% B was used for the first 4-min elution period, followed by 15% B for 0.4 min. For the flush period, 97% B was held for 1 min. The column thermostat was set at 35° C. For the mass spectrometry parameters, the only change from the method described in Amicucci, Galermo et al. 2019 is that the fragmentor voltage was set at 380V. For data analysis, the hydrolysis correction factor was not applied nor needed since the samples contained oligosaccharides instead of polysaccharides. In this analysis method, monosaccharide composition is calculated by quantifying the concentrations of 14 monosaccharides (glucose, galactose, fructose, xylose, arabinose, fucose, rhamnose, glucuronic acid, galacturonic acid, N-acetylglucosamine, N-acetylgalactosamine, mannose, allose, ribose) against their individual standard curves. For example, 30% glucose, as measured by the herein hydrolytic monosaccharide compositional analysis, refers to containing 30 g of glucose per 100 g of the sum of all 14 monosaccharides described above.

As used herein, the term “free monosaccharide compositional analysis” refers to the method described in MJ Amicucci et al. 2019 (Amicucci, Galermo et al. 2019)with some modifications. The derivatization reaction to produce monosaccharides was performed at the optimized condition of 70° C. for 30 minutes. Samples were ran on an Agilent 1290 Infinity II ultra-high performance liquid chromatography (UHPLC) system couple to an Agilent 6490A triple quadrupole (QqQ) mass spectrometer. Separation was carried out on an Agilent InfinityLab Poroshell HPH-C18 column (2.1 mm×50 mm, 1.9 μm particle size) plus a guard column (5 mm) with the same solvent system described in the paper. With a constant flow rate of 1.2 mL/min, an isocratic gradient of 8.5% B was used for the first 4-min elution period, followed by 15% B for 0.4 min. For the flush period, 97% B was held for 1 min. The column thermostat was set at 35° C. For the mass spectrometry parameters, the only change from the method described in the paper is that the fragmentor voltage was set at 380V.For data analysis, the hydrolysis correction factor was not applied since the samples herein contains oligosaccharides instead of polysaccharides. In this analysis method, inherently free unpolymerized monosaccharides are calculated by quantifying the concentrations of 14 monosaccharides (glucose, galactose, fructose, xylose, arabinose, fucose, rhamnose, glucuronic acid, galacturonic acid, N-acetylglucosamine, N-acetylgalactosamine, mannose, allose, ribose) against their individual standard curves. For example, 30% free glucose, as measured by the herein free monosaccharide compositional analysis, refers to containing 30 g of glucose per 100 g of the sum of all 14 monosaccharides described above.

As used herein, the terms “monosaccharide ratio,” “monosaccharide peak area ratio,” “ratio of monosaccharide,” or similar terms can refer to any number of the comparisons dependent upon the relationships observed in the hydrolytic monosaccharide compositional analysis. Absolute concentrations of each monosaccharide were calculated on a relative percent basis in relation to the summation of all other monosaccharides observed. Monosaccharide ratios were calculated by dividing one contributing monosaccharide by any other monosaccharide within the composition.

As used herein, the terms “glycosidic linkage composition,” “glycosidic linkage analysis,” “permethylated linkage composition analysis,” or similar terms, refer to a method described in Galermo, Nandita et al. 2018, hereby incorporated by reference in its entirety for all purposes, with some modifications. The permethylation reaction time was 30 min instead. Samples were ran on an Agilent 1290 Infinity II UHPLC system couple to an Agilent 6490A QqQ mass spectrometer. Separation was carried out on an Agilent InfinityLab Poroshell HPH-C18 column (2.1 mm×100 mm, 1.9 μm particle size) plus a guard column (5 mm) with the same solvent system described in Galermo, Nandita et al. 2018. With a constant flow rate of 0.8 mL/min, an isocratic gradient of 14% B was used for the 16-min elution period, followed by a 2-min 99% B flush period. The column thermostat was set at 35° C. The glycosidic linkage composition is calculated by integrating the chromatographic peak area of all peaks with the following m/z values: 481.2, 495.2, 509.2, 523.3, 525.2, 537.3, 539.3, 553.3, 567.3, 581.3. For example, 20% 4-galactose, as measured by the permethylated linkage composition analysis, refers to the peak area of 4-galactose being 20% of the sum of the peak area of all linkage peaks with the m/z values listed above.

As used herein, the term “other minor linkages” refers to the sum of linkages which are either not entirely annotated or constitute less than 2% of any samples. Therefore, the contributions of these linkages to the sample glycosidic linkage composition are summed into this “other minor linkages” category.

As used herein, the terms “linkage ratio,” “linkage peak area ratio,” “ratio of linkage,” or other similar terms can refer to any number of comparisons dependent upon the relationships observed in the glycosidic linkage composition analysis. Peak area for each linkage was calculated on a relative percent basis of the peak area in relationship to the summation of all other linkage peaks areas observed. Peak area ratios were calculated by dividing one contributing linkage by any other linkage of the same monosaccharide within the composition.

As used herein, the terms “oligosaccharide analysis” or “oligosaccharide composition analysis” (or similar terms) refer to a HPLC-quadrupole time-of-flight (Q-TOF) method described in Amicucci, Nandita et al. 2020, hereby incorporated by reference in its entirety for all purposes, with some modifications. For sample prep, oligosaccharides were reduced by incubation with 2.0 M NaBH4 for 1 h at 65° C. Oligosaccharides were purified using C-18 cartridge 96-well plates: plates were washed with 100% ACN, and the oligosaccharides were loaded and eluted with water. Oligosaccharides were subsequently purified using porous graphitized carbon (PGC) 96-well plates: PCG plates were washed with 80% acetonitrile and 0.1% (v/v) TFA in water, and the oligosaccharides from C-18 purification were loaded and washed with water. The oligosaccharides were eluted with 40% acetonitrile with 0.05% (v/v) TFA. Samples were completely dried by evaporative centrifugation and reconstituted for mass spectrometry analysis. Instrumentation was performed on an Agilent 1260 Infinity II HPLC coupled to an Agilent 6530 Q-TOF mass spectrometer. Using the same stationary (plus a 5 mm guard column) and mobile phase as described in the paper, separation was carried out using the following gradient: 2-15% B, 0-20 min; 15-60% B; 20-45 min. The column thermostat was set at 35° C. The fragmentor voltage was set at 75V. In this method, “oligosaccharide weight %” or “oligo wt. %” or such terms when used in the context of the “oligosaccharide analysis” is calculated by dividing the chromatographic peak area of a particular oligosaccharide by the total peak area of all oligosaccharides identified in that sample during the defined chromatographic period. Generally, when an oligosaccharide composition is described herein to contain a specified weight percent of oligosaccharides on a dry basis having a degree of polymerization of a specified number (e.g., at least 50 wt. % oligosaccharides on a dry basis having a degree of polymerization of between 3 and 50 monosaccharide subunits), such values can be calculated with the aid of the oligosaccharide analysis described herein; however, other methods can also aid this determination, such as size exclusion chromatography using a universal detector, or other methods known in the art.

As used herein, the term “retention factor” refers to the ratio obtained by dividing the retention time of a given peak observed in an oligosaccharide analysis (e.g., HPLC spectrum) by the first oligosaccharide peak (i.e., the lowest retention time) observed in the oligosaccharide analysis.

As used herein the terms, “NMR HSQC Analysis,” “1H-13C HSQC NMR,” “HSQC spectra” or other similar terms correspond to the data generated from two-dimensional spectral analysis of a sample via a Heteronuclear Single Quantum Coherence (HSQC) spin coupling of protons and bonded carbons present in said sample. HSQC experimentation depends on the solvation of samples in a deuterated solvent such as D6-DMSO or D2O. An HSQC spectrum contains a unique peak for each proton attached to the heteronuclear carbon atom being considered, allowing for identification of molecular structure of analyzed sample. Each experiment was conducted with a Bruker AVANCE 600 MHz NMR using heteronuclear single quantum coherence (HSQC) to illustrate the correlation between the 1H and 13C chemical shifts through 1JCH coupling. The resulting FIDs were processed using Bruker TopSpin 4.1.3 and the experimental chemical shifts were utilized to determine oligosaccharide structures and the anomeric characteristics of the glycosidic bonds with the aid of the CASPER program. Relative ratios between alpha and beta bonds were calculated through examination of the 2D 1H-13C HSQC via examination of signal strength in Hz. These values were then compared to determine percent abundance of each linkage type among the same carbohydrate. NMR samples were dried via lyophilization, and the resulting material were then dissolved in 0.75 mL of dimethyl sulfoxide-d6 (DMSO-d6) with a 0.03% (v/v) TMS internal standard at a concentration of 20 mg/mL at a 4.5-6 pH range.

As used herein, “enhances microbial production” refers to a biologically relevant increase in production of a particular metabolite or group of metabolites. In some aspects, a biologically relevant increase is a statistically significant change as measured by parametric or non-parametric tests. In some aspects, a biologically relevant increase can be measured in feces, serum, urine, or organ tissue. In some aspects, a biologically relevant increase is measured through a host metabolic product (choline can be measured as TMA or TMAO). In some aspects, a biologically relevant increase is a 10% increase or a 100% increase or a 500% increase or a 1,000% increase or more. In some aspects the biologically relevant increase can be in the absolute amount of a metabolite or group of metabolites. In some aspects, the biologically relevant increase can be the rate that a metabolite or group of metabolites are produced. In some aspects, the biologically relevant increase can be in the relative amount of a metabolite or group of metabolites.

As used herein, “decreases microbial production” refers to a biologically relevant decrease in the production of a particular metabolite or group of metabolites. In some aspects, a biologically relevant decrease is a statistically significant change as measured by parametric or non-parametric tests. In some aspects, a biologically relevant decrease can be measured in feces, serum, urine, or organ tissue. In some aspects, a biologically relevant decrease is measured through a host metabolic product (choline can be measured as TMA or TMAO). In some aspects, a biologically relevant decrease is a 10% decrease or a 20% decrease or a 50% decrease or a 75% decrease or a 90% decrease or more. In some aspects, the biologically relevant decrease can be in the absolute amount of a metabolite or group of metabolites. In some aspects, the biologically relevant decrease can be the rate that a metabolite or group of metabolites are produced. In some aspects, the biologically relevant decrease can be in the relative amount of a metabolite or group of metabolites.

As used herein, “decreases microbial utilization” refers to a biologically relevant increase in the amount of a particular metabolite or group of metabolites due to lower microbial utilization. In some aspects, a biologically relevant increase is a statistically significant change as measured by parametric or non-parametric tests. In some aspects, a biologically relevant increase can be measured in feces, serum, urine, or organ tissue. In some aspects, a biologically relevant increase is measured through a host metabolic product (choline can be measured as TMA or TMAO). In some aspects, a biologically relevant increase is a 10% increase or a 100% increase or a 500% increase or a 1,000% increase or more. In some aspects the biologically relevant increase can be in the absolute amount of a metabolite or group of metabolites. In some aspects, the biologically relevant increase can be the rate that a metabolite or group of metabolites are produced. In some aspects, the biologically relevant increase can be in the relative amount of a metabolite or group of metabolites.

As used herein, “slows microbial utilization” refers to a biologically relevant increase in the amount or build up of a particular metabolite or group of metabolites due to slowed microbial utilization. In some aspects, a biologically relevant increase is a statistically significant change as measured by parametric or non-parametric tests. In some aspects, a biologically relevant increase can be measured in feces, serum, urine, or organ tissue. In some aspects, a biologically relevant increase is measured through a host metabolic product (choline can be measured as TMA or TMAO). In some aspects, a biologically relevant increase is a 10% increase or a 100% increase or a 500% increase or a 1,000% increase or more. In some aspects the biologically relevant increase can be in the absolute amount of a metabolite or group of metabolites. In some aspects, the biologically relevant increase can be the rate that a metabolite or group of metabolites are produced. In some aspects, the biologically relevant increase can be in the relative amount of a metabolite or group of metabolites.

As used herein, “increases abundance of” refers to a biologically relevant increase in the population of a certain bacterial taxa.

In some aspects, a “biologically relevant increase” is a statistically significant change as measured by parametric or non-parametric tests, generally in reference to the effects of a method comprising administering an oligosaccharide composition or formulation thereof to a subject, or in an in vitro context, relative to an otherwise identical method that does not include administering the oligosaccharide composition or formulation thereof. In some aspects, a biologically relevant increase can be measured in feces, jejunum, cecum, ileum, stomach, large intestines, deuodenum, mouth, respiratory tract, skin, urogenital tract, vaginal tract, or other microbial community. In some aspects, a biologically relevant increase is a 10% increase or a 5× increase or a 10× increase or a 50× increase or a 100× or 1,000× increase or 10,000× or more. In some aspects the biologically relevant increase can be in the absolute amount of a taxa or group of taxa, or amount of a given species (e.g., short chain fatty acid, GLP-1, etc.). In some aspects, the biologically relevant increase can be the rate that a taxa, group of taxa, or other given species increases in the microbial community or in a subject (or location therein, such as a GI tract). In some aspects, the biologically relevant increase can be in the relative amount of a taxa, group of taxa, or other given species in a microbial community or in a subject (or location therein, such as a GI tract). In some aspects, an increase in abundance refers to the presence of one microbial taxa as compared to another microbial taxa, or one given species compare to another given species. “Biologically relevant decrease,” “biologically relevant change,” “biologically relevant amount,” and similar such terms can be similarly understood.

As used herein, “stimulates” in reference to a receptor means a given species (e.g., butyrate, propionate, etc.) acts as an agonist by binding to the receptor, which initiates an immune response.

As used herein, “increases the production” of a given species (e.g., a short chain fatty acids, GLP-1, etc.) means a biologically relevant increase in the production of the given species.

As used herein, “inhibits histone deacetylases” means the enzymatic activity of histone deacetylase is reduced by a biologically relevant amount or even eliminated.

As used herein, “increases the expression of” in reference to a gene (e.g., Muc2, occluding, claudin-4, ZO-1, etc.) means expression of the gene is increased by a biologically relevant amount.

As used herein, “lowers A1c levels” means a biologically relevant decrease in A1c levels.

As used herein, “lowers an inflammatory gastrointestinal marker” means a biologically relevant decrease in the amount of inflammatory gastrointestinal marker.

As used herein, “increases an anti-inflammatory gastrointestinal marker” means a biologically relevant increase in the amount of anti-inflammatory gastrointestinal marker.

As used herein, “lowers inflammation” in reference to a subject or GI tract of a subject means a decrease in one or more of TNF-α, IL1-β, IL-6, or other known inflammatory markers, or any combination thereof, when compared to the levels of the same markers in a subject that is not subjected to the relevant administering or treatment steps with a formulation or oligosaccharide composition described herein.

As used herein, “decreases intestinal barrier permeability” in reference to a subject or GI tract of a subject means (1) a decrease in TEER values, and/or (2) an increase in the expression of one or more of Muc2, occluding, claudin-4, ZO-1 genes, or and/or (3) other known intestinal barrier permeability markers, or (4) any combination thereof, when compared to the same markers in a subject that is not subjected to the relevant administering or treatment steps with a formulation or oligosaccharide composition described herein.

Therapy” means treatment given or action taken to reduce or eliminate symptoms of a disease or pathological condition.

As used herein, a “therapeutically effective amount” or “effective amount” of the disclosed compounds (e.g., oligosaccharide, oligosaccharide composition, and/or synthetic composition) is a dosage of the compound that is sufficient to achieve a desired therapeutic or other outcome or effect, such as an anti-inflammatory effect, stimulation of growth of specified microbiota, and so forth. For example, a therapeutically effective amount of a compound may be such that the subject receives a dosage of about 0.1 μg/kg body weight/day to about 1000 mg/kg body weight/day, for example, a dosage of about 1 μg/kg body weight/day to about 1000 μg/kg body weight/day, such as a dosage of about 5 μg/kg body weight/day to about 500 μg/kg body weight/day. The compound(s) herein may administered in one or more doses, such as on a regular basis, including once-a-day, twice-a-day, every two days, weekly, or bi-weekly for a specified time period in order to achieve and/or maintain the desired therapeutic effect.

As used herein, “treatment” or “treat” refers to a therapeutic intervention that ameliorates a sign or symptom of a disease or pathological condition after it has begun to develop, and also includes addressing a medical condition or disease with the objective of improving or stabilizing an outcome in the subject being treated or addressing an underlying nutritional need. “Treatment” or “treat” therefore include the dietary or nutritional management of the medical condition or disease by addressing nutritional needs of the person being treated. “Treating,” “treat,” and “treatment” have grammatically corresponding meanings. As used herein, the term “ameliorating,” with reference to a disease or pathological condition, refers to any observable beneficial effect of the treatment. The beneficial effect can be evidenced, for example, by a delayed onset of clinical symptoms of the disease or condition in a susceptible subject, a reduction in severity of some or all clinical symptoms of the disease or condition, a slower progression of the disease or condition, an improvement in the overall health or well-being of the subject, or by other parameters well known in the art that are specific to the particular disease or condition. The phrases “treating a disease,” “treating a condition,” and similar terms are inclusive of inhibiting the full development of a disease or condition, for example, in a subject who is at risk for a disease or condition, or who has a disease or condition, such as inflammatory bowel disease (IBD), Crohn's disease, ulcerative colitis, bacterial vaginosis, cardiovascular disease, chronic kidney disease, a nervous system disorder, allergic reaction, atopic dermatitis, and so forth. Preventing a disease or condition refers to prophylactically administering a composition to a subject who does not exhibit signs of a disease or condition, or exhibits only early signs of the disease or condition, for the purpose of decreasing the risk of developing a pathology or condition, or diminishing the severity of a pathology or condition. “Preventive treatment” or “prevention” means treatment given or action taken to diminish the risk of onset or recurrence of a disease. “Primary prevention” means prevention of the initial onset of a condition in an individuals. “Secondary prevention” means, in a subject who has a condition or who has had a condition, (i) prevention of reoccurrence of the condition, (ii) increase in the duration of remission of the condition, and/or (iii) reduction in severity of symptoms of the condition.

“Emotional disorder” means a mental disorder involving a primary disturbance of emotions resulting in the emotions being distorted or inconsistent with circumstances. Emotional disorders include excessive anxiety, fear, anger, happiness, etc.

“Mood disorder” means a mental disorder involving a primary disturbance of a mood resulting in the mood being distorted or inconsistent with circumstances. Mood disorders include depression, major depressive disorder, dysthymia and bipolar disorder.

As used herein, the term “enteral administration” means any form for delivery of a composition to a subject that causes the deposition of the composition in the gastrointestinal tract (including the stomach). Methods of enteral administration include feeding through a naso-gastric tube or jejunum tube, oral, sublingual and rectal.

As used herein, “gastrointestinal tract” or “GI tract” means the passageway in the digestive system of a subject that includes all components from the esophagus to the anus (inclusive), as well as everything situated along the passageway including the stomach, intestines, and so forth. Generally, “gastrointestinal tract” is used interchangeably herein with the term “gut.”

As used herein, the term “microbiota”, “microflora” and “microbiome” mean a community of living microorganisms that typically inhabits a bodily organ or part, for example the gastro-intestinal or urogenital organs of complex organisms, such as mammals and humans. In particular, the most dominant members of the gastrointestinal microbiota include microorganisms of the phyla of Firmicutes, Bacteroidetes, Actinobacteria, Proteobacteria, Synergistetes, Verrucomicrobia, Fusobacteria, and Euryarchaeota; at genus level Bacteroides, Faecalibacterium, Bifidobacterium, Roseburia, Alistipes, Collinsella, Blautia, Coprococcus, Ruminococcus, Eubacterium and Dorea; at species level Bacteroides unformis, Alistipes putredinis, Parabacteroides merdae, Ruminococcus bromii, Dorea longicatena, Bacteroides caccae, Bacteroides thetaiotaomicron, Eubacterium hallil, Ruminococcus torques, Faecalibacterium prausnitzii, Ruminococcus lactaris, Collinsella aerofaciens, Dorea formicigenerans, Bacteroides vulgatus and Roseburia intestinalis. The gastrointestinal microbiota includes the mucosa-associated microbiota, which is located in or attached to the mucous layer covering the epithelium of the gastrointestinal tract, and luminal-associated microbiota, which is found in the lumen of the gastrointestinal tract. Dominant members of the urogenital microbiota include Lactobacillus crispatus, Lactobacillus jensenii, Lactobacillus gasseri, Lactobacillus iners, and Lactobacillus vaginalis.

The term “Bifidobacterium” and its synonyms refer to a genus of anaerobic bacteria having beneficial properties for humans. Members of the Bifidobacterium genus are some of the major strains that make up the gut microbiome, the bacteria that reside in the gastrointestinal tract and have health benefits for their hosts (Guarner and Malagelada 2003).

As used herein, the terms “modulate,” “modulating,” or other similar terms refer to the ability of a disclosed compound (e.g., oligosaccharide or oligosaccharide composition) to alter the amount, degree, or rate of a biological function (including metabolite production), the progression of a disease, or amelioration of a condition. For example, modulating can refer to the ability of a compound to increase or decrease the abundance of a microorganism, increase or decrease production of a metabolite, or elicit a decrease in the inflammation, pain, incidence, or severity of a symptom associated with a particular condition or disease (e.g., associated with the gastrointestinal system, cardiovascular system, renal system, nervous system, immune system, and/or urogenital system).

As used herein, the term “modulation of microbiota” means exerting a modifying or controlling influence on microbiota, for example, an influence leading to an increase in the indigenous intestinal abundance of one or more types of microorganism, such as Bifidobacterium, and/or a metabolite producing bacteria, such as those that produce butyrate. In another example, the influence may lead to a reduction of the intestinal abundance of one or more types of microorganisms, such as Ruminococcus gnavus and/or Proteobacteria.

As used herein, the term “oral administration” means any form for the delivery of a composition to a subject through the mouth. Accordingly, oral administration is a form of enteral administration.

As used herein, the term “EP or Ethanol Precipitate” means a composition which is artificially prepared via the selective precipitation by the addition of a known concentration of ethanol and the separation of the non-soluble portion.

As used herein, the term “ES or Ethanol Supernatant” means a composition which is artificially prepared via the selective precipitation by the addition of a known concentration of ethanol and the separation of the soluble portion.

As used herein, “curdlan” is a polysaccharide with a glycosidic linkage composition comprising a β-1,3 glucose backbone.

As used herein, “glucomannan” is a polysaccharide with a glycosidic linkage composition of about 60% β-1,4 mannose and about 40% β-1,4 glucose backbone.

As used herein, “xylan” is a polysaccharide with a glycosidic linkage composition comprising a β-1,4 xylose backbone with about 13% β-1,2 Glucose-4-OMe.

As used herein, “arabinogalactan” is a polysaccharide with a glycosidic linkage composition comprising a β-1,4 xylose backbone with α-1,3 and α-1,2 arabinose branches in about a 1 to 2 ratio.

As used herein, “locust bean gum” is a polysaccharide with a glycosidic linkage composition of about 73% β-1,4 mannose backbone, with about 23% decorated with β-1,4 galactose.

As used herein, “galactan” is a polysaccharide with a glycosidic linkage composition comprising a β-1,4 galactan backbone.

As under herein, “lichenan” is a polysaccharide with a glycosidic linkage composition comprising a β-1,4 glucose backbone with alternating β-1,3 glucose about 33% of the time.

As under herein, “galactomannan” is a polysaccharide with a glycosidic linkage composition comprising a β-1,4 mannose backbone, with about 22% α-1,3 galactose branching.

As used herein, “β-glucan” (also called “beta glucan”) is a polysaccharide with a glycosidic linkage composition comprising a glucose backbone comprising β-1,4 and β-1,3 in about a 4 to 1 ratio.

As used herein, “xyloglucan” is a polysaccharide with a glycosidic linkage composition comprising a β-1,4 glucose backbone with α-1,6 xylose branches

As used herein, “arabinoxylan” is a polysaccharide with a glycosidic linkage composition comprising β-1,4 xylose backbone with α-1,3 and α-1,2 arabinose branches in about a 1 to 2 ratio.

As used herein, “olive” refers to any part of the plant in the genus Olea. “Olive” may refer to Olea europaea, Olea cuspidate, Olea oleaster, Olea cerasiformis (maderensis), Olea guanchica, Olea laperrinei, Olea maroccana, Olea Canarium, or other species. “Olive” may refer but not limited to colors of green, shades of red, brown, or black. “Olive” may refer to by-products of the plant during harvest or food processing, non-limiting examples include olive flowers and their associated parts (Stigma, style, filament, pedals, flora axis, articulation and nectary), Olive ovules, Olive oil, Olive oil press cake, Olive mill waste water, Olive pomace, olive composts or Olive sludges “Olive” may refer to the solid material after roasting, fermentation, hot-water, enzymatic, chemical, alkaline, super critical fluid, organic solvent, acidic, mechanical pressure or pressure based extractions. “olive” may refer to other non-Olea genus, which are colloquially known as Olive.

As used herein, “Ulvaceae (Ulva intestinalis)” refers to any part of the plant in the genus Ulva, Enteronia, Gemina, Letterstedtia, Lobata, Ochlochaete, Percursaria, Phycoseris, Ruthnielsenia, Solenia, Ulvaria, Umbraulva or Enteromorpha. “Ulvaceae Ulva” may refer to Ulva intestinalis, Ulva lactuca or other species. “Ulvaceae ulva” may refer to by-products of the plant during harvest or food processing, non-limiting examples include a flat or a hollow tubular thallus, Ulvaceae ulva leafs, frond or blades, Ulvaceae ulva stripe or Ulvaceae ulva hold fast. “Ulvaceae ulva” may refer to the solid material after roasting, fermentation, hot-water, enzymatic, chemical, alkaline, super critical fluid, sun drying, organic solvent, acidic, mechanical pressure or pressure based extractions. “Ulvaceae ulva” may refer to other non-ulva genus, which are colloquially known as Sea lettuce, gutweed or grass kelp.

As used herein, “Macrocystis pyrifera” refers to any part of the plant in the genus Macrocystis. “Macrocystis pyrifera” may refer Fucus pyrifer L., Laminaria pyrifera (L.) Lamouroux, Macrocystis humboldtii (Bonpland) C.Ag., Macrocystis planicaulis C. Agardh, Macrocystis pyrifera Var. humboldtii, or other species. “Macrocystis pyrifera” may refer to by-products of the plant during harvest or food processing, non-limiting examples include a flat or a hollow tubular thallus, Macrocystis pyrifera blades, Macrocystis pyrifera air bladders (Pneumatocyst), Macrocystis pyrifera stripe, Macrocystis pyrifera sporophylls or Macrocystis pyrifera hold fast. “Macrocystis pyrifera” may refer to the solid material after roasting, fermentation, hot-water, enzymatic, chemical, alkaline, super critical fluid, sun drying, organic solvent, acidic, mechanical pressure or pressure based extractions. “Macrocystis pyrifera” may refer to other non-Macrocystis genus, which are colloquially known as giant kelp, giant bladder kelp, pacific kelp, or large brown algae.

As used herein, “Sugar Cane” refers to any part of the plant in the genus Saccharum. “Sugar Cane” may refer Saccharum officinarum, Saccharum sinense, Saccharum barberi, Saccharum arundinaceum, Saccharum bengalense, Saccharum edule, Saccharum procerum, Saccharum ravennae, Saccharum robustum, Saccharum spontaneum, hybrids of two, three or more species, or other species. “Sugar cane” may refer to by-products of the plant during harvest or food processing, non-limiting examples include, Sugar cane Leaf (barbojo), Sugar can stalks (cane), raw sugarcane cylinders or cubes, sugar cane bagasse, fresh sugar cane juice, Sugar cane molases, Sugar cane rapadura, Sugar cane flour or Processed sugar cane. “Sugar Cane” may refer to the solid material after roasting, fermentation, hot-water, enzymatic, chemical, alkaline, super critical fluid, sun drying, organic solvent, acidic, mechanical pressure or pressure based extractions. “Sugar Cane” may also refer to “Power Cane”. “Sugar Cane” may refer to other non-Saccharum genus, which are colloquially known as Sugar cane.

As used herein, “Carrot” refers to any part of the plant in the genus Daucus. “Carrot” may refer to Daucus carota, Daucus sativus, Caota sativa, or other species. “Carrot” may refer to by-products of the plant during harvest or food processing, non-limiting examples include, carrot flower, carrot stem, carrot seed, carrot leaf, carrot tap root or carrot lateral roots. “Carrot” may refer to the solid material after roasting, fermentation, hot-water, enzymatic, chemical, alkaline, super critical fluid, sun drying, organic solvent, acidic, mechanical pressure or pressure based extractions. “Carrot” may refer to other non-Daucus genus, which are colloquially known as Daucus carota subsp. Sativus, or wild carrot.

As used herein, “Soy” refers to any part of the plant in the genus Glycine or soja. “Soy” may refer to Dolichos soja L., Glycine angustifolia Miq., Glycine gracilis Skvortsov, Glycine hispida (Moench) Maxim., Glycine soja, Phaseolus max L., Soja angustifolia, Soja hispida Moench, Soja japonica Savi, Soja max, Soja soja H., Soja viridis or other species. “Soy” may refer to by-products of the plant during harvest or food processing, non-limiting examples include, Soy root, soy stem, soy leaves, soy flowers, soy fruiting pods, soy bean, soy protein, soy okra (pulp or curd), soy fiber or soy bean testa. “Soy” may refer to the solid material after roasting, fermentation, hot-water, enzymatic, chemical, alkaline, super critical fluid, sun drying, organic solvent, acidic, mechanical pressure or pressure based extractions. “Soy” may refer to other non-Glycine or soja genus, which are colloquially known as soy bean, kongbiji or soya.

As used herein, “Spingomonas elodea Extract” refers to any part of the bacteria in the genus Sphingomonas. “Spingomonas elodea Extract” may refer to Pseudomonas elodea or other species. “Spingomonas elodea” may refer to by-products of the bacteria during harvest or food processing, non-limiting examples include, extracellular polysaccharides, intracellular polysaccharides, Spingomonas elodea cell wall, Spingomonas elodea carbohydrate membrane, or purified Spingomonas elodea gellen gum polysaccharides. “Spingomonas elodea Extract” may refer to the solid material after roasting, fermentation, hot-water, enzymatic, chemical, alkaline, super critical fluid, sun drying, organic solvent, acidic, mechanical pressure or pressure based extractions. “Spingomonas elodea Extract” may refer to other non-Sphingomonas which are colloquially known as gellen gum, bacteria extract or gelling agent.

As used herein, “coffee” refers to any part of the plant in the genus Coffea. “Coffee” may refer to Coffea arabica, Coffea, robusta, Coffea liberica, or other species. “Coffee” may refer to by-products of the plant during harvest or food processing, non-limiting examples include spent coffee grounds, coffee extracts, coffee beans, coffee parchment coffee pulp, coffee berries, coffee cherries, coffee husk, coffee silver skin, coffee pectin layer, coffee bean outer skin, coffee hulls, coffee leaves, coffee roots, coffee stems, or coffee leaves. “Coffee” may refer to the solid material after roasting, fermentation, hot-water, enzymatic, chemical, alkaline, super critical fluid, organic solvent, acidic, mechanical pressure or pressure based extractions. “Coffee” may refer to other non Coffea genus, which are colloquially known as coffee.

As used herein, “Xanthomonas campestris Extract” refers to any part of the bacteria in the genus Xanthomonas. “Xanthomonas campestris Extract” may refer to extracts from Xanthomonas campestris pv. armoraciae, Xanthomonas campestris pv. begoniae A, Xanthomonas campestris pv. begoniae B, Xanthomonas campestris pv. campestris, Xanthomonas campestris pv. cannabis, Xanthomonas campestris pv. carota, Xanthomonas campestris pv. corylina, Xanthomonas campestris pv. dieffenbachiae, Xanthomonas campestris pv. glycines syn. Xanthomonas axonopodis pv. glycines, Xanthomonas campestris pv. graminis, Xanthomonas campestris pv. hederae, Xanthomonas campestris pv. hyacinthi, Xanthomonas campestris pv. juglandis, Xanthomonas campestris pv. malvacearum or Xanthomonas citri subsp. malvacearum, Xanthomonas campestris pv. musacearum, Xanthomonas campestris pv. mangiferaeindicae, Xanthomonas campestris pv. mori, Xanthomonas campestris pv. nigromaculans, Xanthomonas campestris pv. pelargonii, Xanthomonas campestris pv. phaseoli, Xanthomonas campestris pv. poinsettiicola, Xanthomonas campestris pv. pruni, Xanthomonas campestris pv. raphani, Xanthomonas campestris pv. sesami, Xanthomonas campestris pv. tardicrescens, Xanthomonas campestris pv. translucens, Xanthomonas campestris pv. vesicatoria, Xanthomonas campestris pv. Viticola or other species. “Xanthomonas campestris Extract” may refer to by-products of the bacteria during harvest or food processing, non-limiting examples include, Xanthomonas campestris extracellular polysaccharides, Xanthomonas campestris intracellular polysaccharides, Xanthomonas campestris cell wall, Xanthomonas campestris carbohydrate membrane, or purified Xanthomonas campestris xantham gum polysaccharides. “Xanthomonas campestris Extract” may refer to the solid material after roasting, fermentation, hot-water, enzymatic, chemical, alkaline, super critical fluid, sun drying, organic solvent, acidic, mechanical pressure or pressure based extractions. “Xanthomonas campestris Extract” may refer to other non-Xanthomonas which are colloquially known as xantham gum, bacteria extract or gelling agent.

As used herein, “Pea” refers to any part of the plant in the genus Pisum, Cajanus, lathyrus or Vigina. “Pea” may refer to Pisum sativum, Cajanus cajanor, Vigna unguiculata, Lathyrus aphaca or other species. “Pea” may refer to by-products of the plant during harvest or food processing, non-limiting examples include Pea Powder, Pea pods, Pea flower, Pea stem, Pea stipules, Pea root, Pea seeds, Pea fiber, or crude pea protein. “Pea” may refer to the solid material after roasting, fermentation, hot-water, enzymatic, chemical, alkaline, super critical fluid, sun drying, organic solvent, acidic, mechanical pressure or pressure based extractions. “Pea” may refer to other non Pisum, Cajanus, lathyrus or Vigina genus, which are colloquially known as Pea, Snow pea, split pea, snap pea, field pea or sugar pea.

As used herein, “Tomato” refers to any part of the plant in the genus Solanum. “Tomato” may refer to Solanum lycopersicum, Lycopersicon lycopersicum, Lycopersicon esculentum or other species. “Tomato” may refer to by-products of the plant during harvest or food processing, non-limiting examples include tomato peels, tomato berries, tomato stalks, tomato flowers, tomato seeds, tomato berry flesh, or tomato root. “Tomato” may refer to the solid material after roasting, fermentation, hot-water, enzymatic, chemical, alkaline, super critical fluid, sun drying, organic solvent, acidic, mechanical pressure or pressure based extractions. “Tomato” may refer to other non-Lupinus Solanum, which are colloquially known as tomatoes.

As used herein, “Sacchyromyces cerevisiae” refers to any part of the yeast in the genus Saccharomyces. “Sacchyromyces Cerevisiae” may refer to Saccharomyces cerevisiae or other species. “Sacchyromyces Cerevisiae” may refer to by-products of the yeast cell during harvest or food processing, non-limiting examples include yeast cell membrane, yeast growth media, yeast extracellular polysaccharides, yeast intracellular polysaccharides, yeast cell extracts, yeast fiber, yeast polysaccharides, mannose rich yeast extracts, or yeast spores. “Sacchyromyces Cerevisiae” may refer to the solid material after roasting, fermentation, hot-water, enzymatic, chemical, alkaline, super critical fluid, sun drying, organic solvent, acidic, mechanical pressure or pressure based extractions. “Sacchyromyces Cerevisiae ”may refer to other non-Saccharomyces genus, which are colloquially known as baker yeast.

As used herein, “Yeast beta glucan” refers to a beta glucan found in the cell walls of yeast.” Yeast beta glucan” refers to a polysaccharide containing beta-linked glucose units that may be in the beta-3 position, the beta-4 position, or the beta-6 position. “Yeast beta glucan” may be found alongside other polymers such as mannans. “Yeast beta glucan” can refer to a structure, wherein, the backbone is beta-3 linked and the beta-6 linkages are long branches. “Yeast beta glucan” can be derived from Sacchyromyces cerevisiae or other yeast within or outside of the Sacchyromyces genus. “Yeast beta glucan” can refer to the solid material after roasting, fermentation, hot-water, enzymatic, chemical, alkaline, super critical fluid, sun drying, organic solvent, acidic, mechanical pressure or pressure based extractions.

As used herein, “Tragacanth gum” refers to any part of the yeast in the genus Astragalus. “Tragacanth gum” may refer to Astragalus adscendens, Astragalus gummifer, Astragalus brachycalyx, and Astragalus tragacantha or other species. “Tragacanth gum” may refer to by-products of the tragacanth plant during harvest or food processing, non-limiting examples include Tragacanth sap, Tragacanth powder, Tragacanth beans, Tragacanth leafs or Tragacanth bark. “Tragacanth gum” may refer to the solid material after roasting, fermentation, hot-water, enzymatic, chemical, alkaline, super critical fluid, sun drying, organic solvent, acidic, mechanical pressure or pressure based extractions. “Tragacanth gum” may refer to other non-Astragalus genus, which are colloquially known as Shiraz gum, shiraz, gum elect, Gond Kateera, or gum dragon.

As used herein, “Orange” refers to any part of the plant in the genus citrus. “Orange” may refer to Citrus maxima, Citrus reticulata, Citrus sinensis, Citrus aurantium, Citrus bergamia Risso, Citrus trifoliata or other distinct species, varieties and hybrids. “Orange” may refer to by-products of the plant during harvest or food processing, non-limiting examples include Orange Rind, Orange pith Orange Pulp, Orange fiber, Orange Juice, Orange seeds, Orange leaves, Orange bark, or Orange flowers. “Orange” may refer to the solid material after roasting, fermentation, hot-water, enzymatic, chemical, alkaline, super critical fluid, sun drying, organic solvent, acidic, mechanical pressure or pressure based extractions. “Orange” may refer to other non-citrus genus, which are colloquially known as sweet orange, bitter orange, Bergamot orange, Trifoliate orange or mandarin orange.

As used herein, “Beets” refers to any part of the plant in the genus Beta. “Beets” may refer to Beta vulgarisor or other distinct species and subspecies, adanesisi, maritima, vulgaris, altissima, circla, flavescens, conditiva and crassa. “Beets” may refer to by-products of the plant during harvest or food processing, non-limiting examples include Beet tap roots, beet stems, beet leaves, beet powder, beet fiber, and beetroots. “Beets” may refer to the solid material after roasting, fermentation, hot-water, enzymatic, chemical, alkaline, super critical fluid, sun drying, organic solvent, acidic, mechanical pressure, candied, pickling or pressure based extractions. “Beets” may refer to other non-beta genus, which are colloquially known as sugar beets, sea beets, spinach beets, swiis chard, beet root, table beets, garden beet, red beet, dinner beat golden beet or mangelwurzel.

As used herein, “Baobob” refers to any part of the plant in the genus Adansonia. “Baobob” may refer to Adansonia digitata, Adansonia grandidieri, Adansonia gregorii, Adansonia madagascariensis, Adansonia perrieri, Adansonia rubrostipa, Adansonia suarezensis, Adansonia za or other distinct species. “Baobob” may refer to by-products of the plant during harvest or food processing, non-limiting examples include Baobob Fruit, Baobob powder, Baobob bark, Baobob leaves, Baobob fiber, Baobob seads, Baobob fruit pith, or Baobob flowers. “Baobob” may refer to the solid material after roasting, fermentation, hot-water, enzymatic, chemical, alkaline, super critical fluid, sun drying, organic solvent, acidic, mechanical pressure, or pressure based extractions. “Baobob” may refer to other non-Adansonia genus, which are colloquially known as boab, bottle tree, dead rat tree, monkey-bread tree or montane.

As used herein, “Karaya gum” refers to any part of the plant in the genus Sterculia. “Karaya gum” may refer to Sterculia urens, Cavallium urens, Clompanus urens, Kavalama urens or other distinct species. “Karaya gum” may refer to by-products of the plant during harvest or food processing, non-limiting examples include Karaya sap, Karaya powder, Karaya leaves, or Sterculia urens bark. “Karaya gum” may refer to the solid material after roasting, fermentation, hot-water, enzymatic, chemical, alkaline, super critical fluid, sun drying, organic solvent, acidic, mechanical pressure, or pressure based extractions. “Karaya gum” may refer to other non-Sterculia genus, which are colloquially known as Indian tragacanth gum, katira, kulu or gum sterculia.

As used herein, “lupin glactan” refers to any part of the plant in the genus lupinous. “Lupin” may refer to Lupinus arboreus, Lupinus hirsutus, Lupinus chamissonis, Lupinus albifrons, Lupinus excubitus, lupinous albus, lupinous mutabilis, Lupinus longifolius, Lupinous angustifolius or other distinct species. “Lupin Galactan” may refer to by-products of the plant during harvest or food processing, non-limiting examples include Lupin Bean, Lupin powder, Lupin sead, Lupin flower, lupin stem, “protein extracted” lupin, Lupin fiber, Defatted lupin flour. “Lupin Galactan” may refer to the solid material after roasting, fermentation, hot-water, enzymatic, chemical, alkaline, super critical fluid, sun drying, organic solvent, acidic, mechanical pressure, or pressure-based extractions. “Lupin” may refer to other non-lupinus genus, which are colloquially known as lupin beans, white lupin, tarwi, chocho, kirku, turmus or blue lupin

Any viscosity measurement or property reported herein employs water as the solvent, unless specified otherwise.

As used herein, when a table, spectrum, or other data is referred to as representing the features or properties possessed by a particular composition, oligosaccharide, or other compound or mixture, unless specified otherwise, the same analysis method and procedure used to obtain the table, spectrum, or other data is to be used to determine the properties of the particular composition, oligosaccharide, or other compound or mixture.

Various oligosaccharide compositions disclosed herein are identified by a “CLX” designation. Such CLX compositions can be prepared in any suitable manner and by any suitable method, including ground up synthetic methods (e.g., oligomerizing monomeric or shorter chain oligosaccharides into the indicated oligosaccharides), or by depolymerization methods (e.g., by depolymerizing polysaccahrides or longer chain oligosaccharides into shorter chain oligosaccharides). For example, in some aspects, the CLX compositions disclosed herein can be prepared by a depolymerization method disclosed in WO 2018/236917 (Amicucci et al., “Production of bioactive oligosaccharides”) or WO 2021/097138 (Amicucci et al., “High-yield peroxide quench-controlled polysaccharide depolymerization and compositions thereof”), both of which are hereby incorporated by reference in their entireties for all purposes. By way of example, such CLX compositions disclosed herein can be prepared by a method comprising dissolving the indicated source polysaccharide(s) (e.g., microbial curdlan, lichenan, xylan, etc.) in 20 ml of HPLC grade water in a capped reaction vessel and placed in a shaker-incubator for 20 min at 55° C. and 85 RPM. The pH of the solution was adjusted to 5.2. Hydrogen peroxide (5 ml) and iron (II) sulfate or copper (II) sulfate (in either case, 2.75 mg in 50 μL water) were added to the reaction mixture and mixed thoroughly. The reaction in the capped reaction vessel proceeded in the shaker-incubator at 55° C. and 65 RPM for two hours. The capped reaction cooled to 12° C. in a −20° C. freezer. Ammonium hydroxide (1 ml of 28% v/v to pH 10.2) was used to adjust pH and sample was reacted at 45° C. in a shaker-incubator for 1 hour at 20 RPM, the cap was left loose to allow oxygen, ammonia, and carbon dioxide gases to be released. The sample is then frozen and lyophilized, then stored at −80° C. The freeze-dried oligosaccharide mixture was rehydrated with the minimum amount of water required to allow for a free-flowing solution. This solution was then loaded onto a column containing 15 mL mixed bed ion exchange resin per gram (dry weight) of crude material, and the runoff was collected in a plastic freezer bag. Once the material was loaded onto the column, the column was then rinsed with 3 bed volumes of water. Finally, the runoff was sealed and frozen in the bag, then carefully shattered and subjected to lyophilization.

For NMR analysis of the CLX compositions, oligosaccharides were dissolved in D₂O or D₆-DMSO at a concentration of 50 mg/ml and were analyzed on a 600 MHz Bruker NMR spectrometer for their HSQC spectra.

As used herein, the term “CLX101” refers to an oligosaccharide composition wherein about 99% of the mass comprises glucose, as measured by hydrolytic monosaccharide compositional analysis. For composition CLX101, the glycosidic linkage composition comprises about 75% 3-linked glucose, about 9% terminal glucose, and about 15% other minor linkages. The CLX101 composition comprises, approximately, the 1H-13C HSQC NMR correlations set forth in Table A for CLX101. The CLX101 composition comprises, approximately, the values set forth in in Table B, as measured by oligosaccharide analysis. CLX 101 has a dynamic viscosity at 25° C. at 100 mg/ml of about 1.306 mPa*s. CLX 101 is derived from microbial curdlan. CLX 101 generally is derived from microbial curdlan, but can also be derived from other materials/sources (e.g., depolymerization of polysaccharides or oligomerization of lower DP mono- and/or oligo-saccharides) that provide oligosaccharides that have the same (or substantially the same, e.g., values within 10%, or within 15%, or within 20%, or within 25%, or within 30%) dynamic viscosity, hydrolytic monosaccharide composition, glycosidic linkage composition, oligosaccharide analysis, and 1H-13C HSQC NMR analysis as CLX101.

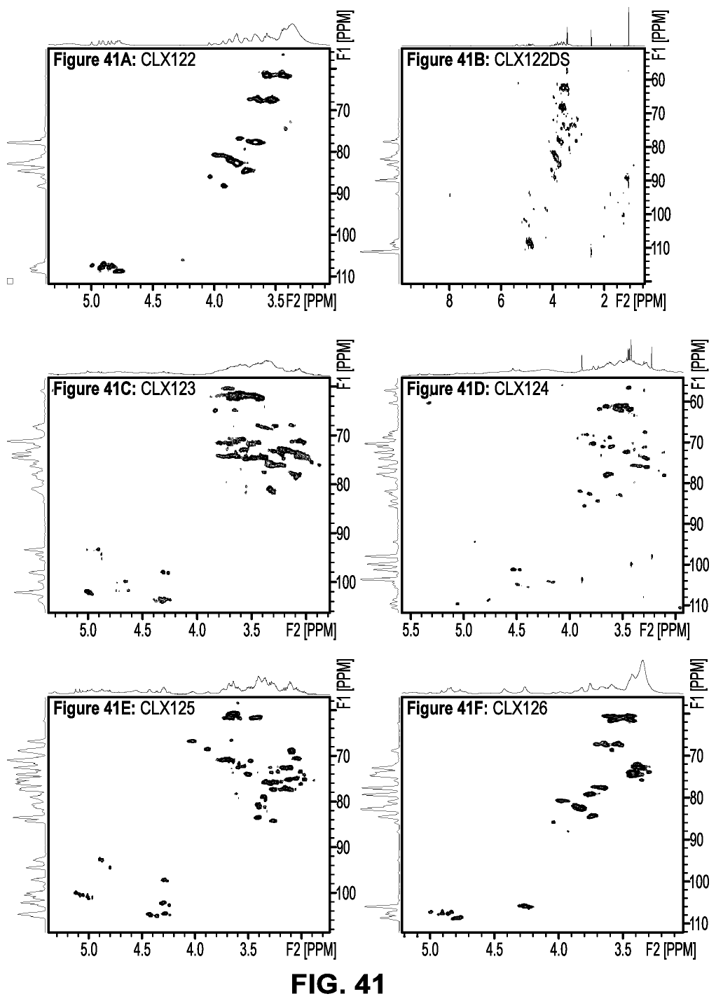

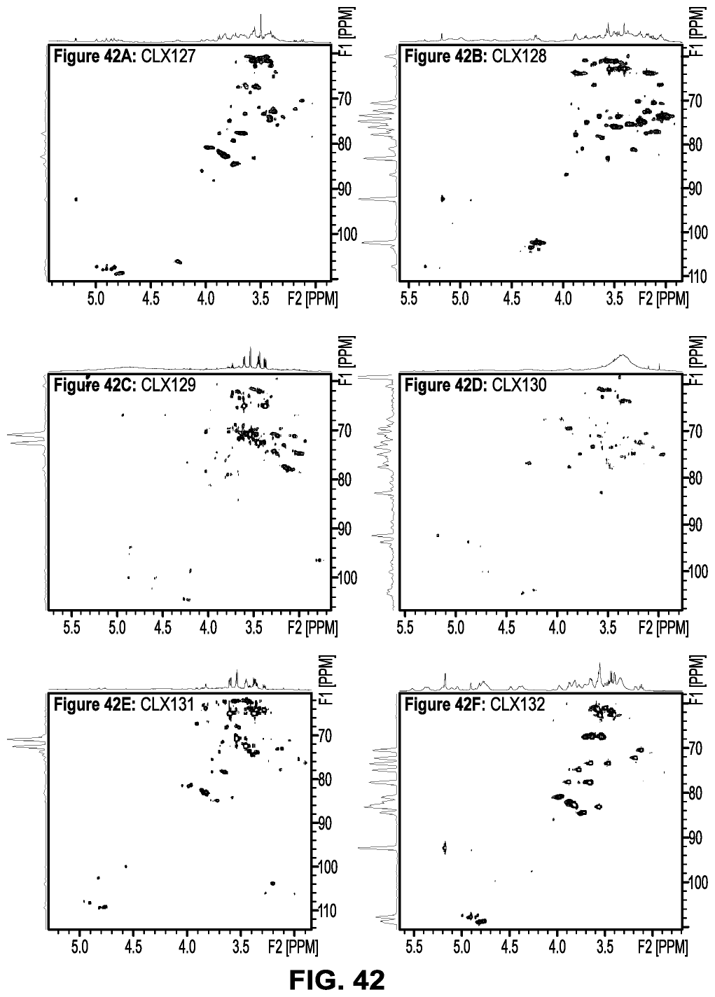

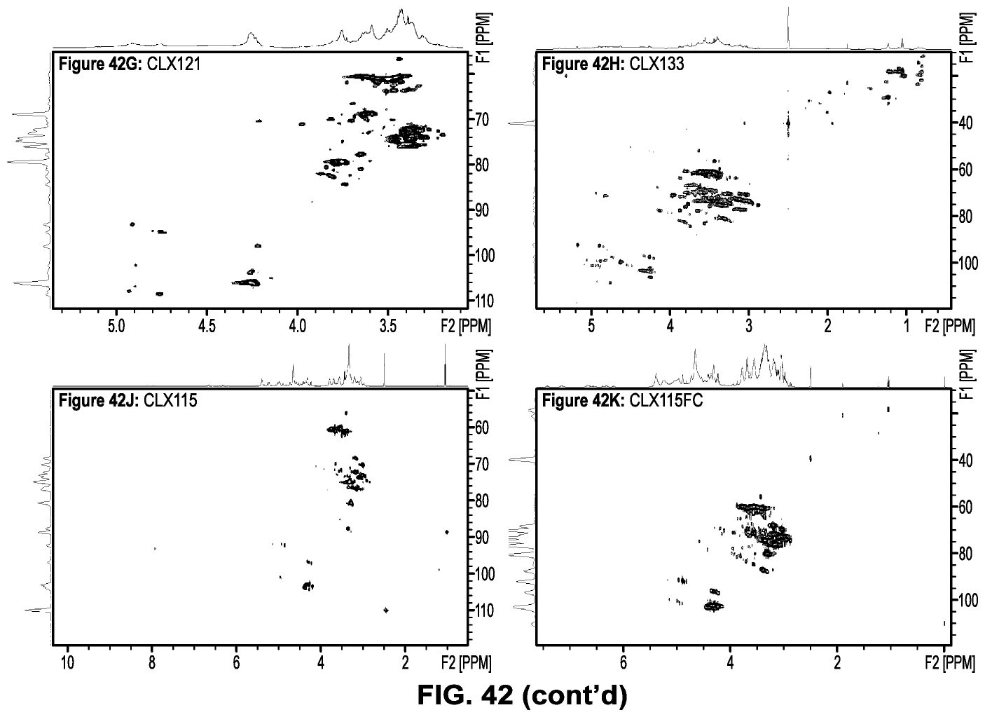

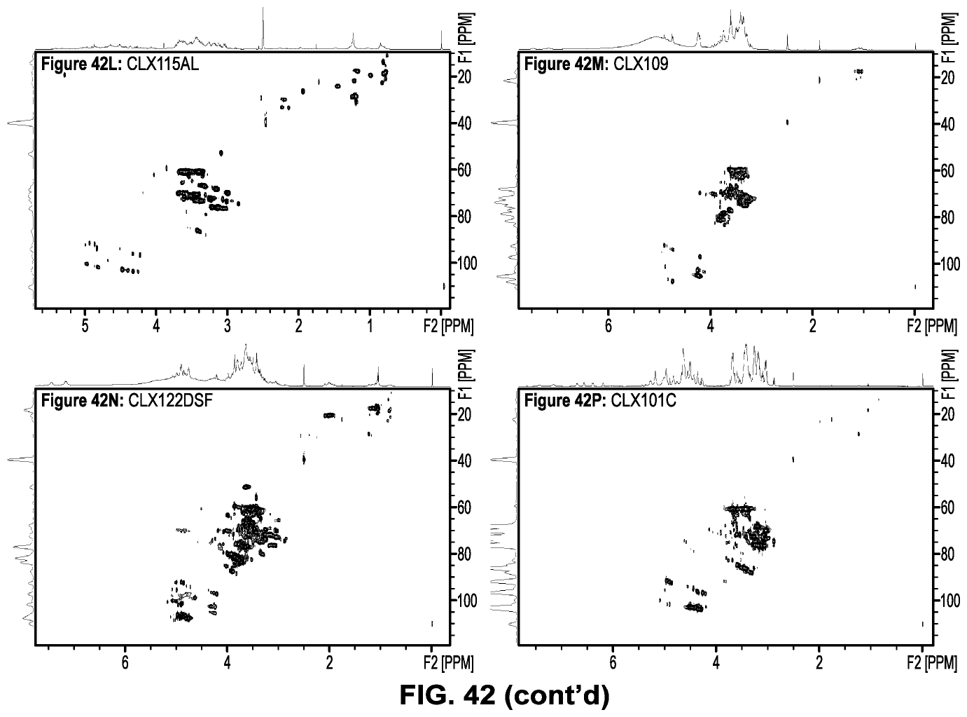

As used herein, the term “CLX101C” refers to an oligosaccharide composition wherein about 99% of the mass comprises glucose, as measured by hydrolytic monosaccharide compositional analysis. For composition CLX101C, the glycosidic linkage composition comprises about 75% 3-linked glucose, about 9% terminal glucose, and about 15% other minor linkages. The CLX101C composition comprises, approximately, the 1H-13C HSQC NMR correlations set forth in Table A for CLX101C (see also the spectrum in FIG. 42P). The CLX101C composition comprises, approximately, the values set forth in in Table B2, as measured by oligosaccharide analysis. CLX 101C has a dynamic viscosity at 25° C. at 100 mg/ml of about 1.307 mPa*s. CLX101C generally is derived from microbial curdlan, but can also be derived from other materials/sources (e.g., depolymerization of polysaccharides or oligomerization of lower DP mono- and/or oligo-saccharides) that provide oligosaccharides that have the same (or substantially the same, e.g., values within 10%, or within 15%, or within 20%, or within 25%, or within 30%) dynamic viscosity, hydrolytic monosaccharide composition, glycosidic linkage composition, oligosaccharide analysis and 1H-13C HSQC NMR analysis as CLX101C.

As used herein, the term “CLX102” refers to an oligosaccharide composition wherein about 37% of the mass comprises glucose, and about 60% of the mass comprises mannose, as measured by hydrolytic monosaccharide compositional analysis. For composition CLX102, the glycosidic linkage composition comprises about 32% 4-linked glucose, about 8% terminal glucose, about 48% 4-linked mannose, and about 13% terminal mannose. The CLX102 composition comprises, approximately, the 1H-13C HSQC NMR correlations set forth in Table A for CLX102. The CLX102 composition comprises, approximately, the values set forth in Table C, as measured by oligosaccharide analysis. CLX 102 has a dynamic viscosity at 25° C. at 100 mg/ml of about 1.392 mPa*s. CLX102 generally is derived from Konjac glucomannan, but can also be derived from other materials/sources (e.g., depolymerization of polysaccharides or oligomerization of lower DP mono- and/or oligo-saccharides) that provide oligosaccharides that have the same (or substantially the same, e.g., values within 10%, or within 15%, or within 20%, or within 25%, or within 30%) dynamic viscosity, hydrolytic monosaccharide composition, glycosidic linkage composition, oligosaccharide analysis, and 1H-13C HSQC NMR analysis as CLX102.

As used herein, the term “CLX103” refers to an oligosaccharide composition wherein about 85% of the mass comprises xylose, about 5% of the mass comprises glucose, about 5% of the mass comprises mannose, and about 2% of the mass comprises galactose, as measured by hydrolytic monosaccharide compositional analysis. For composition CLX103, the glycosidic linkage composition comprises, approximately, about 14% 4-linked glucose, about 5% terminal glucose, about 55% 4-linked xylose, about 7% terminal xylose, and about 15% 4-mannose. CLX103 composition comprises, approximately, the 1H-13C HSQC NMR correlations set forth in Table A for CLX103. The CLX103 composition comprises, approximately, the values set forth in Table D, as measured by oligosaccharide analysis. CLX 103 has a dynamic viscosity at 25° C. at 100 mg/ml of about 1.093 mPa*s. CLX 103 generally is derived from beechwood xylan, but can also be derived from other materials/sources (e.g., depolymerization of polysaccharides or oligomerization of lower DP mono- and/or oligo-saccharides) that provide oligosaccharides that have the same (or substantially the same, e.g., values within 10%, or within 15%, or within 20%, or within 25%, or within 30%) dynamic viscosity, hydrolytic monosaccharide composition, glycosidic linkage composition, oligosaccharide analysis, and 1H-13C HSQC NMR analysis as CLX103.

As used herein, the term “CLX105” refers to an oligosaccharide composition wherein about 87% of the mass comprises galactose and about 6% of the mass comprises arabinose, as measured by hydrolytic monosaccharide compositional analysis. For composition CLX105, the glycosidic linkage composition comprises about 17% 3-linked galactose, 14% 3,6-linked galactose, 12% 6-linked galactose, about 51% terminal galactose, and about 3% terminal arabinose. CLX105 composition comprises, approximately, the 1H-13C HSQC NMR correlations set forth in Table A for CLX105. The CLX105 composition comprises, approximately, the values set forth in Table E, as measured by oligosaccharide analysis. CLX 105 has a dynamic viscosity at 25° C. at 100 mg/ml of about 1.210 mPa*s. CLX 105 generally is derived from arabinogalactan, but can also be derived from other materials/sources (e.g., depolymerization of polysaccharides or oligomerization of lower DP mono- and/or oligo-saccharides) that provide oligosaccharides that have the same (or substantially the same, e.g., values within 10%, or within 15%, or within 20%, or within 25%, or within 30%) dynamic viscosity, hydrolytic monosaccharide composition, glycosidic linkage composition, oligosaccharide analysis, and 1H-13C HSQC NMR analysis as CLX105.

As used herein, the term “CLX108” refers to an oligosaccharide composition wherein about 73% of the mass comprises mannose and about 23% of the mass comprises galactose, as measured by hydrolytic monosaccharide compositional analysis. For composition CLX108, the glycosidic linkage composition comprises about 20% terminal galactose, about 62% 4-linked mannose, about 9% 4,6-linked mannose, and about 7% terminal mannose. The CLX108 composition comprises, approximately, the 1H-13C HSQC NMR correlations set forth in Table A for CLX108. The CLX108 composition comprises, approximately, the values set forth in Table F, as measured by oligosaccharide analysis. CLX 108 has a dynamic viscosity at 25° C. at 100 mg/ml of about 6.447 mPa*s. CLX 108 generally is derived from locust bean gum, but can also be derived from other materials/sources (e.g., depolymerization of polysaccharides or oligomerization of lower DP mono- and/or oligo-saccharides) that provide oligosaccharides that have the same (or substantially the same, e.g., values within 10%, or within 15%, or within 20%, or within 25%, or within 30%) dynamic viscosity, hydrolytic monosaccharide composition, glycosidic linkage composition, oligosaccharide analysis, and 1H-13C HSQC NMR analysis as CLX108.

As used herein, the term “CLX109” refers to an oligosaccharide composition wherein about 80% of the mass comprises galactose, about 9% of the mass comprises arabinose, about 5% of the mass comprises rhamnose, and about 3% of the mass comprises galacturonic acid, as measured by hydrolytic monosaccharide compositional analysis. For composition CLX109, the glycosidic linkage composition comprises about 62% 4-linked galactose, about 34% terminal galactose, and about 2% terminal arabinose. The CLX109 composition comprises, approximately, the 1H-13C HSQC NMR correlations set forth in Table A for CLX109 (see also the spectrum in FIG. 42M). The CLX109 composition comprises, approximately, the values set forth in Table G, as measured by oligosaccharide analysis. CLX 109 has a dynamic viscosity at 25° C. at 100 mg/ml of about 0.830 mPa*s. CLX 109 generally is derived from potato pectic galactan, but can also be derived from other materials/sources (e.g., depolymerization of polysaccharides or oligomerization of lower DP mono- and/or oligo-saccharides) that provide oligosaccharides that have the same (or substantially the same, e.g., values within 10%, or within 15%, or within 20%, or within 25%, or within 30%) dynamic viscosity, hydrolytic monosaccharide composition, glycosidic linkage composition, and oligosaccharide analysis as CLX109.

As used herein, the term “CLX110” refers to an oligosaccharide composition wherein about 80% of the mass comprises glucose, about 9% of the mass comprises mannose, and about 9% of the mass comprises galactose, as measured by hydrolytic monosaccharide compositional analysis. For composition CLX110, the glycosidic linkage composition comprises about 26% 3-linked glucose, 43% 4-linked glucose, about 23% terminal glucose, and about 7% terminal galactose. CLX110 composition comprises, approximately, the 1H-13C HSQC NMR correlations set forth in Table A for CLX110. The CLX110 composition comprises, approximately, the values set forth in Table H, as measured by oligosaccharide analysis. CLX 110 has a dynamic viscosity at 25° C. at 100 mg/ml of about 1.250 mPa*s. CLX 110 generally is derived from lichenan, but can also be derived from other materials/sources (e.g., depolymerization of polysaccharides or oligomerization of lower DP mono- and/or oligo-saccharides) that provide oligosaccharides that have the same (or substantially the same, e.g., values within 10%, or within 15%, or within 20%, or within 25%, or within 30%) dynamic viscosity, hydrolytic monosaccharide composition, glycosidic linkage composition, oligosaccharide analysis, and 1H-13C HSQC NMR analysis as CLX110.

As used herein, the term “CLX111” refers to an oligosaccharide composition wherein about 78% of the mass comprises mannose and about 19% of the mass comprises galactose, as measured by hydrolytic monosaccharide compositional analysis. For composition CLX111, the glycosidic linkage composition comprises about 2% 4-linked glucose, about 18% terminal galactose, about 47% 4-linked mannose, about 7% 4,6-linked mannose, and about 20% terminal mannose. The CLX111 composition comprises, approximately, the 1H-13C HSQC NMR correlations set forth in in Table A for CLX111. The CLX111 composition comprises, approximately, the values set forth in Table I, as measured by oligosaccharide analysis. CLX 111 has a dynamic viscosity at 25° C. at 100 mg/ml of about 1.683 mPa*s. CLX 111 generally is derived from carob galactomannan, but can also be derived from other materials/sources (e.g., depolymerization of polysaccharides or oligomerization of lower DP mono- and/or oligo-saccharides) that provide oligosaccharides that have the same (or substantially the same, e.g., values within 10%, or within 15%, or within 20%, or within 25%, or within 30%) dynamic viscosity, hydrolytic monosaccharide composition, glycosidic linkage composition, oligosaccharide analysis, and 1H-13C HSQC NMR analysis as CLX111.

As used herein, the term “CLX112” refers to an oligosaccharide composition wherein about 97% of the mass comprises glucose, as measured by hydrolytic monosaccharide compositional analysis. For composition CLX112, the glycosidic linkage composition comprises about 17% 3-linked glucose, 49% 4-linked glucose, and about 31% terminal glucose. The CLX112 composition comprises, approximately, the 1H-13C HSQC NMR correlations set forth in Table A for CLX112. The CLX112 composition comprises, approximately, the values set forth in Table J, as measured by oligosaccharide analysis. CLX 112 has a dynamic viscosity at 25° C. at 100 mg/ml of about 1.248 mPa*s. CLX 112 generally is derived from barley beta glucan, but can also be derived from other materials/sources (e.g., depolymerization of polysaccharides or oligomerization of lower DP mono- and/or oligo-saccharides) that provide oligosaccharides that have the same (or substantially the same, e.g., values within 10%, or within 15%, or within 20%, or within 25%, or within 30%) dynamic viscosity, hydrolytic monosaccharide composition, glycosidic linkage composition, oligosaccharide analysis, and 1H-13C HSQC NMR analysis as CLX112.

As used herein, the term “CLX113” refers to an oligosaccharide composition wherein about 49% of the mass comprises glucose, about 36% of the mass comprises xylose, and about 14% comprises galactose, as measured by hydrolytic monosaccharide compositional analysis. For composition CLX113, the glycosidic linkage composition comprises about 28% 4-linked glucose, about 6% 6-linked glucose, about 20% 4,6-linked glucose, about 4% terminal glucose, about 21% terminal galactose, about 6% 2-linked xylose, and about 11% terminal xylose. The CLX113 composition comprises, approximately, the 1H-13C HSQC NMR correlations set forth in Table A for CLX113. The CLX113 composition comprises, approximately, the values set forth in Table K, as measured by oligosaccharide analysis. CLX 113 has a dynamic viscosity at 25° C. at 100 mg/ml of about 1.209 mPa*s. CLX 113 generally is derived from tamarind seed xyloglucan, but can also be derived from other materials/sources (e.g., depolymerization of polysaccharides or oligomerization of lower DP mono- and/or oligo-saccharides) that provide oligosaccharides that have the same (or substantially the same, e.g., values within 10%, or within 15%, or within 20%, or within 25%, or within 30%) dynamic viscosity, hydrolytic monosaccharide composition, glycosidic linkage composition, oligosaccharide analysis, and 1H-13C HSQC NMR analysis as CLX113.

As used herein, the term “CLX114” refers to an oligosaccharide composition wherein about 60% of the mass comprises xylose, about 37% of the mass comprises arabinose, and about 2% of the mass comprises galactose, as measured by hydrolytic monosaccharide compositional analysis. For composition CLX114, the glycosidic linkage composition comprises about 31% 4-linked xylose, about 22% 3,4-linked xylose, about 3% terminal xylose, about 31% terminal arabinose, and about 11% other minor linkages. The CLX114 composition comprises, approximately, the 1H-13C HSQC NMR correlations set forth in Table A for CLX114. The CLX114 composition comprises, approximately, the values set forth in Table L, as measured by oligosaccharide analysis. CLX 114 has a dynamic viscosity at 25° C. at 100 mg/ml of about 1.877 mPa*s. CLX 114 generally is derived from Rye arabinoxylan, but can also be derived from other materials/sources (e.g., depolymerization of polysaccharides or oligomerization of lower DP mono- and/or oligo-saccharides) that provide oligosaccharides that have the same (or substantially the same, e.g., values within 10%, or within 15%, or within 20%, or within 25%, or within 30%) dynamic viscosity, hydrolytic monosaccharide composition, glycosidic linkage composition, oligosaccharide analysis, and 1H-13C HSQC NMR analysis as CLX114.

As used herein, the term “CLX115” refers to an oligosaccharide composition wherein about 95% of the mass comprises glucose and about 2% of the mass comprises arabinose, as measured by hydrolytic monosaccharide compositional analysis. For composition CLX115, the glycosidic linkage composition comprises about 64% 4-linked glucose, about 23% 3-linked glucose, and about 13% terminal glucose. The CLX115 composition comprises, approximately, the 1H-13C HSQC NMR correlations set forth in Table A for CLX115 (see also the spectrum in FIG. 42J). The CLX115 composition comprises, approximately, the values set forth in Table M, as measured by oligosaccharide analysis. CLX115 has a dynamic viscosity of about 1.382 mPa*s at 100 mg/ml at 25° C. CLX 115 generally is derived from oat beta glucan, but can also be derived from other materials/sources (e.g., depolymerization of polysaccharides or oligomerization of lower DP mono- and/or oligo-saccharides) that provide oligosaccharides that have the same (or substantially the same, e.g., values within 10%, or within 15%, or within 20%, or within 25%, or within 30%) dynamic viscosity, hydrolytic monosaccharide composition, glycosidic linkage composition, and oligosaccharide analysis as CLX115.

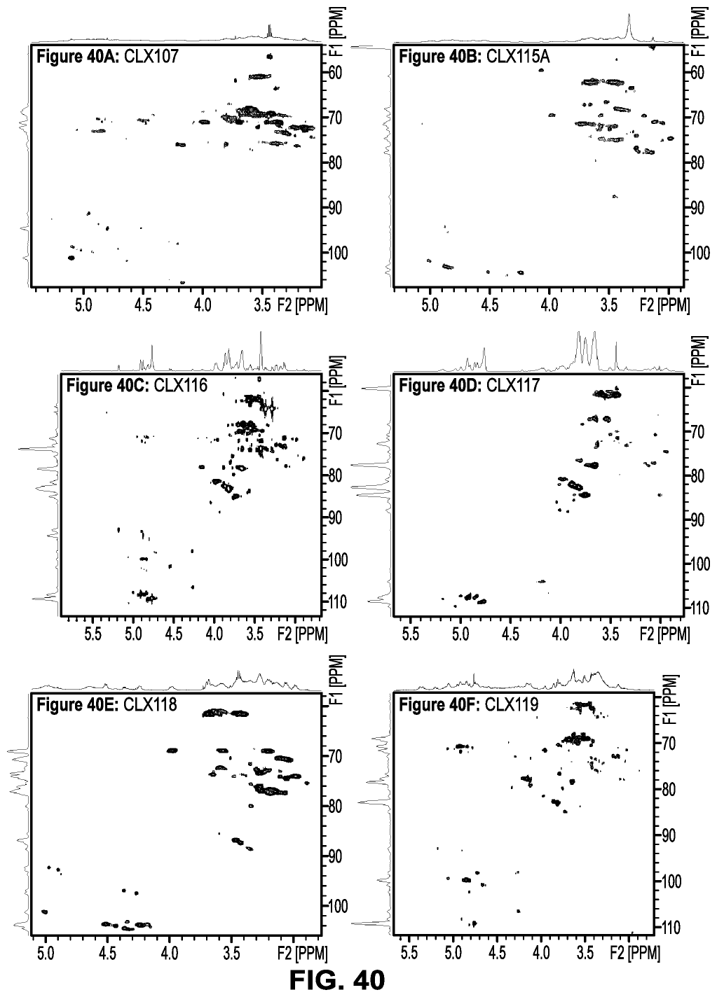

As used herein, the term “CLX 107” refers to an oligosaccharide composition wherein about 42% of the mass comprises galactose, about 37% of the mass comprises glucose, and about 16% of the mass comprises rhamnose, as measured by hydrolytic monosaccharide compositional analysis. For composition CLX 107, the glycosidic linkage composition comprises, approximately, the amounts described in Table N for CLX 107. The CLX 107 composition comprises, approximately, the 1H-13C HSQC NMR correlations set forth in Table O for CLX 107 (see also the spectrum in FIG. 40A). The CLX 107 composition has a dynamic viscosity at 25° C. at 100 mg/ml of about 2.012 mPa*s. CLX 107 generally is derived from Karaya Gum, but can be derived from any source or method (e.g., depolymerization of polysaccharides or oligomerization of lower DP mono- and/or oligo-saccharides) that provides oligosaccharides that have the same (or substantially the same, e.g., values within 10%, or within 15%, or within 20%, or within 25%, or within 30%) dynamic viscosity, hydrolytic monosaccharide composition, glycosidic linkage composition, and 1H-13C HSQC NMR analysis as CLX 107.

As used herein, the term “CLX 115A” refers to an oligosaccharide composition wherein about 71% of the mass comprises mannose and about 29% of the mass comprises glucose, as measured by hydrolytic monosaccharide compositional analysis. For composition CLX 115A, the glycosidic linkage composition comprises, approximately, the amounts set forth in Table N for CLX 115A. The CLX 115A composition comprises, approximately, the 1H-13C HSQC NMR correlations set forth in Table O for CLX 115A (see also the spectrum in FIG. 40B). The CLX 115A oligosaccharide composition comprises, approximately, the values set forth in Table P, as measured by oligosaccharide analysis. CLX 115A has a dynamic viscosity at 25° C. at 100 mg/ml of about 2.997 mPa*s. CLX 115A generally is derived from Saccharomyces cerevisiae, but can be derived from any source or method (e.g., depolymerization of polysaccharides or oligomerization of lower DP mono- and/or oligo-saccharides) that provides oligosaccharides that have the same (or substantially the same, e.g., values within 10%, or within 15%, or within 20%, or within 25%, or within 30%) dynamic viscosity, hydrolytic monosaccharide composition, oligosaccharide analysis, glycosidic linkage composition, and 1H-13C HSQC NMR analysis as CLX 115A.

As used herein, the term “CLX 116” refers to an oligosaccharide composition wherein about 51% of the mass comprises arabinose, about 11% of the mass comprises glucose, about 9% of the mass comprises galactose, about 9% of the mass comprises rhamnose, about 9% of the mass comprises galacturonic acid, and about 7% of the mass comprises glucuronic acid, as measured by hydrolytic monosaccharide compositional analysis. For composition CLX 116, the glycosidic linkage composition comprises, approximately, the amounts set forth in Table N for CLX 116. The CLX 116 composition comprises, approximately, the 1H-13C HSQC NMR correlations set forth in Table O for CLX 116 (see also the spectrum in FIG. 40C). The CLX 116 oligosaccharide composition comprises, approximately, the values set forth in Table EE, as measured by oligosaccharide analysis. CLX 116 has a dynamic viscosity at 25° C. at 100 mg/ml of about 2.516 mPa*s. CLX 116 generally is derived from Oranges, but can be derived from any source or method (e.g., depolymerization of polysaccharides or oligomerization of lower DP mono- and/or oligo-saccharides) that provides oligosaccharides that have the same (or substantially the same, e.g., values within 10%, or within 15%, or within 20%, or within 25%, or within 30%) dynamic viscosity, hydrolytic monosaccharide composition, oligosaccharide analysis, glycosidic linkage composition, and 1H-13C HSQC NMR analysis as CLX 116.

As used herein, the term “CLX 117” refers to an oligosaccharide composition wherein about 60% of the mass comprises arabinose, about 19% of the mass comprises galactose, about 6% of the mass comprises glucose, about 6% of the mass comprises xylose, about 4% of the mass comprises glucuronic acid, about 3% of the mass comprises rhamnose, and about 2% of the mass comprises fucose, as measured by hydrolytic monosaccharide compositional analysis. For composition CLX 117, the glycosidic linkage composition comprises, approximately, the amounts set forth in Table N. The CLX 117 composition comprises, approximately, the 1H-13C HSQC NMR correlations set forth in Table O for CLX 117 (see also the spectrum in FIG. 40D). The CLX 117 oligosaccharide composition comprises, approximately, the values set forth in Table Q, as measured by oligosaccharide analysis. CLX 117 has a dynamic viscosity at 25° C. at 100 mg/ml of about 2.860 mPa*s. CLX 117 generally is derived from Tragacanth gum, but can be derived from any source or method (e.g., depolymerization of polysaccharides or oligomerization of lower DP mono- and/or oligo-saccharides) that provides oligosaccharides that have the same (or substantially the same, e.g., values within 10%, or within 15%, or within 20%, or within 25%, or within 30%) dynamic viscosity, hydrolytic monosaccharide composition, glycosidic linkage composition, and 1H-13C HSQC NMR analysis as CLX 117.

As used herein, the term “CLX 118” refers to an oligosaccharide composition wherein about 96% of the mass comprises glucose, as measured by hydrolytic monosaccharide compositional analysis. For composition CLX 118, the glycosidic linkage composition comprises, approximately, the amounts set forth in Table N for CLX 118. The CLX 118 composition comprises, approximately, the 1H-13C HSQC NMR correlations set forth in Table O for CLX 118 (see also the spectrum in FIG. 40E). The CLX 118 oligosaccharide composition comprises, approximately, the values set forth in Table R, as measured by oligosaccharide analysis. CLX 118 has a dynamic viscosity at 25° C. at 100 mg/ml of about 1.267 mPa*s. CLX 118 generally is derived from Yeast derived beta glucans, but can be derived from any source or method (e.g., depolymerization of polysaccharides or oligomerization of lower DP mono- and/or oligo-saccharides) that provides oligosaccharides that have the same (or substantially the same, e.g., values within 10%, or within 15%, or within 20%, or within 25%, or within 30%) dynamic viscosity, hydrolytic monosaccharide composition, oligosaccharide analysis, glycosidic linkage composition, and 1H-13C HSQC NMR analysis as CLX 118.

As used herein, the term “CLX 119” refers to an oligosaccharide composition wherein about 33% of the mass comprises arabinose, about 22% of the mass comprises galactose, about 15% of the mass comprises galacturonic acid, about 13% of the mass comprises glucose, about 11% of the mass comprises rhamnose, and about 3% of the mass comprises xylose, as measured by hydrolytic monosaccharide compositional analysis. For composition CLX 119, the glycosidic linkage composition comprises, approximately, the amounts set forth in Table N for CLX 119. The CLX 119 composition comprises, approximately, the 1H-13C HSQC NMR correlations set forth in Table O for CLX 119 (see also the spectrum in FIG. 40F). The CLX 119 oligosaccharide composition comprises, approximately, the values set forth in Table S, as measured by oligosaccharide analysis. CLX 119 has a dynamic viscosity at 25° C. at 100 mg/ml of about 1.728 mPa*s. CLX 119 generally is derived from Tomato, but can be derived from any source or method (e.g., depolymerization of polysaccharides or oligomerization of lower DP mono- and/or oligo-saccharides) that provides oligosaccharides that have the same (or substantially the same, e.g., values within 10%, or within 15%, or within 20%, or within 25%, or within 30%) dynamic viscosity, hydrolytic monosaccharide composition, oligosaccharide analysis, glycosidic linkage composition, and 1H-13C HSQC NMR analysis as CLX 119.

As used herein, the term “CLX121” refers to an oligosaccharide composition wherein about 77% of the mass comprises galactose, about 9% of the mass comprises arabinose, and about 3% of the mass comprises rhamnose, as measured by hydrolytic monosaccharide compositional analysis. For composition CLX121, the glycosidic linkage composition comprises about 61% 4-linked galactose, about 21% terminal galactose, about 5% 5-linked galactose, and about 2% terminal arabinose, shown in Table N. The CLX121 composition comprises, approximately, the 1H-13C HSQC NMR correlations set forth in Table O for CLX121 (see also the spectrum in FIG. 42G). The CLX121 composition comprises, approximately, the values set forth in Table T, as measured by oligosaccharide analysis. CLX 121 has a dynamic viscosity at 25° C. at 100 mg/ml of about 0.830 mPa*s. CLX 121 generally is derived from lupin galactan, but can also be derived from other materials/sources (e.g., depolymerization of polysaccharides or oligomerization of lower DP mono- and/or oligo-saccharides) that provide oligosaccharides that have the same (or substantially the same, e.g., values within 10%, or within 15%, or within 20%, or within 25%, or within 30%) dynamic viscosity, hydrolytic monosaccharide composition, glycosidic linkage composition, and oligosaccharide analysis as CLX121.

As used herein, the term “CLX 122” refers to an oligosaccharide composition wherein about 80% of the mass comprises arabinose, about 10% of the mass comprises galactose, about 4% of the mass comprises glucose, about 4% of the mass comprises galacturonic acid, and about 2% of the mass comprises rhamnose, as measured by hydrolytic monosaccharide compositional analysis. For composition CLX 122, the glycosidic linkage composition comprises, approximately, the amounts set forth in Table N for CLX 122. The CLX 122 composition comprises, approximately, the 1H-13C HSQC NMR correlations set forth in Table O for CLX 122 (see also the spectrum in FIG. 41A). The CLX 122 oligosaccharide composition comprises, approximately, the values set forth in Table U, as measured by oligosaccharide analysis. CLX 122 has a dynamic viscosity at 25° C. at 100 mg/ml of about 2.913 mPa*s. CLX 122 generally is derived from Pea, but can be derived from any source or method (e.g., depolymerization of polysaccharides or oligomerization of lower DP mono- and/or oligo-saccharides) that provides oligosaccharides that have the same (or substantially the same, e.g., values within 10%, or within 15%, or within 20%, or within 25%, or within 30%) dynamic viscosity, hydrolytic monosaccharide composition, oligosaccharide analysis, glycosidic linkage composition, and 1H-13C HSQC NMR analysis as CLX 122.

As used herein, the term “CLX 122DS” refers to an oligosaccharide composition wherein about 77% of the mass comprises arabinose, about 10% of the mass comprises glucose, about 6% of the mass comprises galactose, about 3% of the mass comprises galacturonic acid, and about 3% of the mass comprises rhamnose, as measured by hydrolytic monosaccharide compositional analysis. For composition CLX 122DS, the glycosidic linkage composition comprises, approximately, the amounts set forth in Table N for CLX 122DS. The CLX 122DS composition comprises, approximately, the 1H-13C HSQC NMR correlations set forth in Table O for CLX 122DS (see also the spectrum in FIG. 41B). The CLX 122DS oligosaccharide composition comprises, approximately, the values set forth in Table U2, as measured by oligosaccharide analysis. CLX 122DS has a dynamic viscosity at 25° C. at 100 mg/ml of about 1.565 mPa*s. CLX 122DS generally is derived from Pea, but can be derived from any source or method (e.g., depolymerization of polysaccharides or oligomerization of lower DP mono- and/or oligo-saccharides) that provides oligosaccharides that have the same (or substantially the same, e.g., values within 10%, or within 15%, or within 20%, or within 25%, or within 30%) dynamic viscosity, hydrolytic monosaccharide composition, oligosaccharide analysis, glycosidic linkage composition, and 1H-13C HSQC NMR analysis as CLX 122DS.

As used herein, the term “CLX 123” refers to an oligosaccharide composition wherein about 64% of the mass comprises glucose, about 31% of the mass comprises mannose, and about 3% of the mass comprises glucuronic acid, as measured by hydrolytic monosaccharide compositional analysis. For composition CLX 123, the glycosidic linkage composition comprises, approximately, the amounts set forth in Table N for CLX 123. The CLX 123 composition comprises, approximately, the 1H-13C HSQC NMR correlations set forth in Table O for CLX 123 (see also the spectrum in FIG. 41C). The CLX 123 oligosaccharide composition comprises, approximately, the values set forth in Table V, as measured by oligosaccharide analysis. CLX 123 has a dynamic viscosity at 25° C. at 100 mg/ml of about 1.658 mPa*s. CLX 123 generally is derived from Xanthomonas Campestris extract, but can be derived from any source or method (e.g., depolymerization of polysaccharides or oligomerization of lower DP mono- and/or oligo-saccharides) that provides oligosaccharides that have the same (or substantially the same, e.g., values within 10%, or within 15%, or within 20%, or within 25%, or within 30%) dynamic viscosity, hydrolytic monosaccharide composition, oligosaccharide analysis, glycosidic linkage composition, and 1H-13C HSQC NMR analysis as CLX 123.

As used herein, the term “CLX 124” refers to an oligosaccharide composition wherein about 50% of the mass comprises galactose, about 26% of the mass comprises mannose, about 14% of the mass comprises arabinose, and about 7% of the mass comprises glucose, as measured by hydrolytic monosaccharide compositional analysis. For composition CLX 124, the glycosidic linkage composition comprises, approximately, the amounts set forth in Table N for CLX 124. The CLX 124 composition comprises, approximately, the 1H-13C HSQC NMR correlations set forth in Table O for CLX 124 (see also the spectrum in FIG. 41D). The CLX 124 oligosaccharide composition comprises, approximately, the values set forth in Table W, as measured by oligosaccharide analysis. CLX 124 has a dynamic viscosity at 25° C. at 100 mg/ml of about 1.341 mPa*s. CLX 124 generally is derived from Coffee, but can be derived from any source or method (e.g., depolymerization of polysaccharides or oligomerization of lower DP mono- and/or oligo-saccharides) that provides oligosaccharides that have the same (or substantially the same, e.g., values within 10%, or within 15%, or within 20%, or within 25%, or within 30%) dynamic viscosity, hydrolytic monosaccharide composition, oligosaccharide analysis, glycosidic linkage composition, and 1H-13C HSQC NMR analysis as CLX 124.

As used herein, the term “CLX 125” refers to an oligosaccharide composition wherein about 44% of the mass comprises glucose, about 43% of the mass comprises rhamnose, and about 8% of the mass comprises glucuronic acid, as measured by hydrolytic monosaccharide compositional analysis. For composition CLX 125, the glycosidic linkage composition comprises, approximately, the amount set forth in Table N, for CLX 125. CLX 125 composition comprises, approximately, the 1H-13C HSQC NMR correlations set forth in Table O for CLX 125 (see also the spectrum in FIG. 41E). The CLX 125 oligosaccharide composition comprises, approximately, the values set forth in Table X, as measured by oligosaccharide analysis. CLX 125 has a dynamic viscosity at 25° C. at 100 mg/ml of about 1.525 mPa*s. CLX 125 generally is derived from Spingomonas elodea Extract, but can be derived from any source or method (e.g., depolymerization of polysaccharides or oligomerization of lower DP mono- and/or oligo-saccharides) that provides oligosaccharides that have the same (or substantially the same, e.g., values within 10%, or within 15%, or within 20%, or within 25%, or within 30%) dynamic viscosity, hydrolytic monosaccharide composition, oligosaccharide analysis, glycosidic linkage composition, and 1H-13C HSQC NMR analysis as CLX 125.

As used herein, the term “CLX 126” refers to an oligosaccharide composition wherein about 50% of the mass comprises galactose, about 34% of the mass comprises arabinose, about 5% of the mass comprises xylose, about 3% of the mass comprises galacturonic acid, about 3% of the mass comprises glucose, about 2% of the mass comprises rhamnose, and about 2% of the mass comprises fucose, as measured by hydrolytic monosaccharide compositional analysis. For composition CLX 126, the glycosidic linkage composition comprises, approximately, the amounts set forth in Table N for CLX 126. The CLX 126 composition comprises, approximately, the 1H-13C HSQC NMR correlations set forth in Table O for CLX 126 (see also the spectrum in FIG. 41F). The CLX 126 oligosaccharide composition comprises, approximately, the values set forth in Table Y, as measured by oligosaccharide analysis. CLX 126 has a dynamic viscosity at 25° C. at 100 mg/ml of about 4.425 mPa*s. CLX 126 generally is derived from Soy, but can be derived from any source or method (e.g., depolymerization of polysaccharides or oligomerization of lower DP mono- and/or oligo-saccharides) that provides oligosaccharides that have the same (or substantially the same, e.g., values within 10%, or within 15%, or within 20%, or within 25%, or within 30%) dynamic viscosity, hydrolytic monosaccharide composition, oligosaccharide analysis, glycosidic linkage composition, and 1H-13C HSQC NMR analysis as CLX 126.

As used herein, the term “CLX 127” refers to an oligosaccharide composition wherein about 57% of the mass comprises arabinose, about 26% of the mass comprises galactose, and about 14% of the mass comprises glucose, as measured by hydrolytic monosaccharide compositional analysis. For composition CLX 127, the glycosidic linkage composition comprises, approximately, the amounts set forth in Table N for CLX 127. The CLX 127 composition comprises, approximately, the 1H-13C HSQC NMR correlations set forth in Table O for CLX 127 (see also the spectrum in FIG. 42A). The CLX 127 oligosaccharide composition comprises, approximately, the values set forth in Table Z, as measured by oligosaccharide analysis. CLX 127 has a dynamic viscosity at 25° C. at 100 mg/ml of about 2.350 mPa*s. CLX 127 generally is derived from Carrot, but can be derived from any source or method (e.g., depolymerization of polysaccharides or oligomerization of lower DP mono- and/or oligo-saccharides) that provides oligosaccharides that have the same (or substantially the same, e.g., values within 10%, or within 15%, or within 20%, or within 25%, or within 30%) dynamic viscosity, hydrolytic monosaccharide composition, oligosaccharide analysis, glycosidic linkage composition, and 1H-13C HSQC NMR analysis as CLX 127.

As used herein, the term “CLX 128” refers to an oligosaccharide composition wherein about 47% of the mass comprises xylose, about 35% of the mass comprises glucose, about 12% of the mass comprises arabinose, and about 2% of the mass comprises galactose, as measured by hydrolytic monosaccharide compositional analysis. For composition CLX 128, the glycosidic linkage composition comprises, approximately, the amounts set forth in Table N for CLX 128. The CLX 128 composition comprises, approximately, the 1H-13C HSQC NMR correlations set forth in Table O for CLX 128 (see also the spectrum in FIG. 42B). The CLX 128 oligosaccharide composition comprises, approximately, the values set forth in Table AA, as measured by oligosaccharide analysis. CLX 128 has a dynamic viscosity at 25° C. at 100 mg/ml of about 1.818 mPa*s. CLX 128 generally is derived from Sugar Cane, but can be derived from any source or method (e.g., depolymerization of polysaccharides or oligomerization of lower DP mono- and/or oligo-saccharides) that provides oligosaccharides that have the same (or substantially the same, e.g., values within 10%, or within 15%, or within 20%, or within 25%, or within 30%) dynamic viscosity, hydrolytic monosaccharide composition, oligosaccharide analysis, glycosidic linkage composition, and 1H-13C HSQC NMR analysis as CLX 128.

As used herein, the term “CLX 129” refers to an oligosaccharide composition wherein about 55% of the mass comprises fucose, about 16% of the mass comprises xylose, about 13% of the mass comprises glucose, and about 10% of the mass comprises galactose, as measured by hydrolytic monosaccharide compositional analysis. For composition CLX 129, the glycosidic linkage composition comprises, approximately, the amounts set forth in Table N for CLX 129. The CLX 129 composition comprises, approximately, the 1H-13C HSQC NMR correlations set forth in Table O for CLX 129 (see also the spectrum in FIG. 42C). The CLX 129 oligosaccharide composition comprises, approximately, the values set forth in Table BB, as measured by oligosaccharide analysis. CLX 129 has a dynamic viscosity at 25° C. at 100 mg/ml of about 2.467 mPa*s. CLX 129 generally is derived from Macrocystis pyrifera, but can be derived from any source or method (e.g., depolymerization of polysaccharides or oligomerization of lower DP mono- and/or oligo-saccharides) that provides oligosaccharides that have the same (or substantially the same, e.g., values within 10%, or within 15%, or within 20%, or within 25%, or within 30%) dynamic viscosity, hydrolytic monosaccharide composition, oligosaccharide analysis, glycosidic linkage composition, and 1H-13C HSQC NMR analysis as CLX 129.

As used herein, the term “CLX 130” refers to an oligosaccharide composition wherein about 63% of the mass comprises rhamnose, about 14% of the mass comprises xylose, about 13% of the mass comprises glucose, about 4% of the mass comprises glucuronic acid, and about 4% of the mass comprises galactose, as measured by hydrolytic monosaccharide compositional analysis. For composition CLX 130, the glycosidic linkage composition comprises, approximately, the amounts set forth in Table N for CLX 130. The CLX 130 composition comprises, approximately, the 1H-13C HSQC NMR correlations set forth in Table O for CLX 130 (see also the spectrum in FIG. 42D). CLX 130 has a dynamic viscosity at 25° C. at 100 mg/ml of about 4.244 mPa*s. CLX 130 generally is derived from Ulvaceae (Ulva intestinalis), but can be derived from any source or method (e.g., depolymerization of polysaccharides or oligomerization of lower DP mono- and/or oligo-saccharides) that provides oligosaccharides that have the same (or substantially the same, e.g., values within 10%, or within 15%, or within 20%, or within 25%, or within 30%) dynamic viscosity, hydrolytic monosaccharide composition, oligosaccharide analysis, glycosidic linkage composition, and 1H-13C HSQC NMR analysis as CLX 130.

As used herein, the term “CLX 131” refers to an oligosaccharide composition wherein about 55% of the mass comprises arabinose, about 13% of the mass comprises galactose, about 12% of the mass comprises glucose, about 5% of the mass comprises galacturonic acid, about 4% of the mass comprises xylose, and about 3% of the mass comprises mannose, as measured by hydrolytic monosaccharide compositional analysis. For composition CLX 131, the glycosidic linkage composition comprises, approximately, the amounts set forth in Table N for CLX 131. The CLX 131 composition comprises, approximately, the 1H-13C HSQC NMR correlations set forth in Table O for CLX 131 (see also the spectrum in FIG. 42E). CLX 131 has a dynamic viscosity at 25° C. at 100 mg/ml of about 1.338 mPa*s. CLX 131 generally is derived from Olive, but can be derived from any source or method (e.g., depolymerization of polysaccharides or oligomerization of lower DP mono- and/or oligo-saccharides) that provides oligosaccharides that have the same (or substantially the same, e.g., values within 10%, or within 15%, or within 20%, or within 25%, or within 30%) dynamic viscosity, hydrolytic monosaccharide composition, oligosaccharide analysis, glycosidic linkage composition, and 1H-13C HSQC NMR analysis as CLX 131.

As used herein, the term “CLX 132” refers to an oligosaccharide composition wherein about 53% of the mass comprises galactose, about 15% of the mass comprises arabinose, about 14% of the mass comprises galacturonic acid, about 12% of the mass comprises rhamnose, and about 3% of the mass comprises glucose, as measured by hydrolytic monosaccharide compositional analysis. For composition CLX 132, the glycosidic linkage composition comprises, approximately, the amounts set forth in Table N for CLX 132. The CLX 132 composition comprises, approximately, the 1H-13C HSQC NMR correlations set forth in Table O for CLX 132 (see also the spectrum in FIG. 42F). The CLX 132 oligosaccharide composition comprises, approximately, the values set forth in Table CC. CLX 132 has a dynamic viscosity at 25° C. at 100 mg/ml of about 1.712 mPa*s. CLX 132 generally is derived from Beet, but can be derived from any source or method (e.g., depolymerization of polysaccharides or oligomerization of lower DP mono- and/or oligo-saccharides) that provides oligosaccharides that have the same (or substantially the same, e.g., values within 10%, or within 15%, or within 20%, or within 25%, or within 30%) dynamic viscosity, hydrolytic monosaccharide composition, oligosaccharide analysis, glycosidic linkage composition, and 1H-13C HSQC NMR analysis as CLX 132.

As used herein, the term “CLX 133” refers to an oligosaccharide composition wherein about 35% of the mass comprises glucose, about 18% of the mass comprises galactose, about 12% of the mass comprises galactose, about 12% of the mass comprises galacturonic acid, about 4% of the mass comprises rhamnose, and about 3% of the mass comprises fucose, as measured by hydrolytic monosaccharide compositional analysis. For composition, the glycosidic linkage composition comprises, approximately, the amounts set forth in Table N for CLX 133. The CLX 133 composition comprises, approximately, the 1H-13C HSQC NMR correlations set forth in Table O for CLX 133 (see also the spectrum in FIG. 42H). The CLX 133 oligosaccharide composition comprises, approximately, the values set forth in Table DD. CLX 133 has a dynamic viscosity at 25° C. at 100 mg/ml of about 1.712 mPa*s. CLX 133 generally is derived from Baobob, but can be derived from any source or method (e.g., depolymerization of polysaccharides or oligomerization of lower DP mono- and/or oligo-saccharides) that provides oligosaccharides that have the same (or substantially the same, e.g., values within 10%, or within 15%, or within 20%, or within 25%, or within 30%) dynamic viscosity, hydrolytic monosaccharide composition, oligosaccharide analysis, glycosidic linkage composition, and 1H-13C HSQC NMR analysis as CLX 133.

As used herein, the term “CLX115AL” refers to an oligosaccharide composition wherein about 60% of the mass comprises mannose and about 40% of the mass comprises glucose, as measured by hydrolytic monosaccharide compositional analysis. For composition the glycosidic linkage composition comprises, approximately, the amounts set forth in Table N for CLX115AL. The CLX115AL composition comprises, approximately, the 1H-13C HSQC NMR correlations set forth in Table O for CLX115AL (see also the spectrum in FIG. 42L). The CLX115AL composition comprises, approximately, the values set forth in Table P2, as measured by oligosaccharide analysis. CLX115AL has a dynamic viscosity of about 1.382 mPa*s at 100 mg/ml at 25° C. CLX115AL generally is derived from oat beta glucan, but can also be derived from other materials/sources (e.g., depolymerization of polysaccharides or oligomerization of lower DP mono- and/or oligo-saccharides) that provide oligosaccharides that have the same (or substantially the same, e.g., values within 10%, or within 15%, or within 20%, or within 25%, or within 30%) dynamic viscosity, hydrolytic monosaccharide composition, glycosidic linkage composition, oligosaccharide analysis and 1H-13C HSQC NMR analysis as CLX115AL.

As used herein, the term “CLX 115FC” refers to an oligosaccharide composition wherein about 95% of the mass comprises glucose and about 2% of the mass comprises arabinose, as measured by hydrolytic monosaccharide compositional analysis. For composition CLX115FC, the glycosidic linkage composition comprises about 64% 4-linked glucose, about 23% 3-linked glucose, and about 13% terminal glucose. The CLX 115FC composition comprises, approximately, the 1H-13C HSQC NMR correlations set forth in Table A for CLX 115FC (see also the spectrum in FIG. 42K). The CLX 115FC oligosaccharide composition comprises, approximately, the values set forth in Table M2, as measured by oligosaccharide analysis. CLX 115FC has a dynamic viscosity at 25° C. at 100 mg/ml of about 2.997 mPa*s. CLX 115FC generally is derived from Saccharomyces cerevisiae, but can be derived from any source or method (e.g., depolymerization of polysaccharides or oligomerization of lower DP mono- and/or oligo-saccharides) that provides oligosaccharides that have the same (or substantially the same, e.g., values within 10%, or within 15%, or within 20%, or within 25%, or within 30%) dynamic viscosity, hydrolytic monosaccharide composition, oligosaccharide analysis, glycosidic linkage composition, and 1H-13C HSQC NMR analysis as CLX 115FC.

As used herein, the term “CLX 122DSF” refers to an oligosaccharide composition wherein about 77% of the mass comprises arabinose, about 10% of the mass comprises glucose, about 6% of the mass comprises galactose, about 3% of the mass comprises galacturonic acid, and about 3% of the mass comprises rhamnose, as measured by hydrolytic monosaccharide compositional analysis. For composition CLX 122DSF, the glycosidic linkage composition comprises, approximately, the amounts set forth in Table N. The CLX 122DSF composition comprises, approximately, the 1H-13C HSQC NMR correlations set forth in Table O for CLX 122DSF (see also the spectrum in FIG. 42N). The CLX 122DSF oligosaccharide composition comprises, approximately, the values set forth in Table U3, as measured by oligosaccharide analysis. CLX 122DSF has a dynamic viscosity at 25° C. at 100 mg/ml of about 1.555 mPa*s. CLX 122DSF generally is derived from Pea, but can be derived from any source or method (e.g., depolymerization of polysaccharides or oligomerization of lower DP mono- and/or oligo-saccharides) that provides oligosaccharides that have the same (or substantially the same, e.g., values within 10%, or within 15%, or within 20%, or within 25%, or within 30%) dynamic viscosity, hydrolytic monosaccharide composition, oligosaccharide analysis, 1H-13C HSQC NMR analysis and glycosidic linkage composition as CLX 122DSF.

TABLE A
1H-13C HSQC NMR correlations from oligosaccharide compositions used herein.
The listed pairs correspond to those major peaks in the anomeric region.

| CLX 101 | | CLX 102 | | CLX 111 | | CLX 112 | | CLX 105 | | CLX 110 | |
|---|---|---|---|---|---|---|---|---|---|---|---|
| 1H δ | 13C δ | 1H δ | 13C δ | 1H δ | 13C δ | 1H δ | 13C δ | 1H δ | 13C δ | 1H δ | 13C δ |
| [ppm] | [ppm] | [ppm] | [ppm] | [ppm] | [ppm] | [ppm] | [ppm] | [ppm] | [ppm] | [ppm] | [ppm] |
| 3.37 | 103.44 | 4.52 | 102.44 | 4.50 | 96.76 | 4.64 | 95.88 | 3.54 | 100.74 | 4.51 | 102.45 |
| 3.42 | 99.57 | 4.55 | 96.58 | 4.51 | 96.70 | 4.75 | 102.73 | 3.64 | 99.80 | 4.54 | 102.32 |
| 3.52 | 105.61 | 4.56 | 96.52 | 4.55 | 96.52 | 4.67 | 95.70 | 3.65 | 99.80 | 4.64 | 95.82 |
| 3.52 | 98.05 | 4.62 | 95.88 | 4.57 | 96.46 | 4.76 | 102.68 | 3.66 | 102.68 | 4.66 | 95.75 |
| 3.57 | 103.32 | 4.65 | 95.76 | 4.58 | 96.41 | 4.78 | 102.50 | 3.70 | 93.24 | 4.67 | 95.75 |
| 3.74 | 90.66 | 4.66 | 95.76 | 4.85 | 96.82 | 4.79 | 102.50 | 3.71 | 105.14 | 4.75 | 102.73 |
| 3.76 | 90.61 | 4.76 | 99.98 | 4.87 | 96.76 | 5.23 | 91.89 | 3.71 | 105.08 | 4.79 | 102.52 |
| 3.80 | 84.04 | 4.84 | 96.82 | 4.89 | 93.65 | 4.65 | 95.88 | 3.77 | 90.96 | 5.23 | 91.92 |
| 3.92 | 90.66 | 4.86 | 96.76 | 4.91 | 93.65 | 4.52 | 102.50 | 3.78 | 100.16 | 5.37 | 99.03 |
| 3.95 | 90.66 | 4.88 | 93.65 | 5.00 | 98.34 | 4.51 | 102.50 | 3.86 | 81.93 | 5.42 | 99.72 |
| 4.64 | 95.88 | 4.90 | 93.65 | 5.00 | 94.00 | 4.54 | 102.32 | 3.91 | 103.61 | | |
| 4.65 | 95.88 | 5.17 | 93.77 | 5.03 | 98.69 | 4.66 | 95.70 | 3.91 | 99.22 | | |
| 4.67 | 95.64 | 5.21 | 91.84 | 5.18 | 93.83 | | | 3.93 | 98.63 | | |
| 4.69 | 95.64 | 5.27 | 92.30 | 5.23 | 92.54 | | | 3.96 | 98.57 | | |
| 4.76 | 102.73 | 5.39 | 99.57 | 5.23 | 88.55 | | | 4.05 | 99.22 | | |
| 4.77 | 102.68 | | | 5.24 | 88.55 | | | 4.20 | 98.40 | | |
| 4.80 | 102.50 | | | 5.26 | 92.25 | | | 4.24 | 98.46 | | |
| 4.81 | 102.50 | | | 5.28 | 92.30 | | | 4.45 | 103.44 | | |
| 5.24 | 92.01 | | | 5.29 | 101.39 | | | 4.69 | 103.85 | | |

| CLX 103 | | CLX 113 | | CLX 108 | | CLX 114 | | CLX101C | |
|---|---|---|---|---|---|---|---|---|---|
| 1H δ | 13C δ | 1H δ | 13C δ | 1H δ | 13C δ | 1H δ | 13C δ | 1H δ | 13C δ |
| [ppm] | [ppm] | [ppm] | [ppm] | [ppm] | [ppm] | [ppm] | [ppm] | [ppm] | [ppm] |
| 4.16 | 101.09 | 4.56 | 104.43 | 4.52 | 102.52 | 4.46 | 101.27 | 5.08 | 99.89 |
| 4.31 | 101.15 | 4.56 | 102.45 | 4.53 | 102.52 | 4.48 | 101.56 | 4.99 | 95.49 |
| 4.35 | 109.59 | 4.57 | 96.57 | 4.55 | 96.50 | 4.49 | 101.56 | 4.97 | 91.54 |
| 4.39 | 100.10 | 4.67 | 103.00 | 4.57 | 96.50 | 4.51 | 102.91 | 4.95 | 101.63 |
| 4.40 | 100.74 | 4.67 | 95.68 | 4.64 | 104.30 | 4.51 | 101.56 | 4.94 | 91.32 |
| 4.46 | 101.80 | 4.68 | 95.75 | 4.76 | 100.13 | 4.55 | 102.91 | 4.90 | 91.97 |
| 4.48 | 101.62 | 4.69 | 95.75 | 4.87 | 96.78 | 4.58 | 102.27 | 4.90 | 101.66 |
| 4.48 | 100.86 | 4.70 | 95.68 | 4.91 | 93.63 | 4.58 | 99.92 | 4.85 | 92.50 |
| 4.49 | 102.79 | 4.96 | 98.83 | 5.03 | 99.38 | 4.58 | 96.46 | 4.55 | 94.04 |
| 4.49 | 101.62 | 5.18 | 98.62 | 5.03 | 98.69 | 4.60 | 96.41 | 4.51 | 101.05 |
| 4.49 | 100.86 | 5.24 | 91.79 | 5.03 | 97.80 | 4.61 | 102.27 | 4.51 | 102.83 |
| 4.50 | 102.79 | 5.41 | 92.06 | 5.09 | 107.44 | 4.62 | 96.35 | 4.44 | 94.87 |
| 4.53 | 102.91 | | | 5.18 | 93.77 | 4.63 | 99.86 | 4.42 | 103.20 |
| 4.54 | 102.91 | | | 5.19 | 107.10 | 4.63 | 96.35 | 4.43 | 94.87 |
| 4.55 | 100.21 | | | 5.28 | 92.27 | 5.05 | 108.18 | 4.36 | 103.63 |
| 4.57 | 102.21 | | | 5.41 | 92.13 | 5.19 | 92.01 | 4.36 | 96.11 |
| 4.57 | 100.21 | | | 5.43 | 92.06 | 5.23 | 108.59 | 4.28 | 103.85 |
| 4.58 | 102.21 | | | | | 5.28 | 108.01 | 4.26 | 96.64 |
| 4.58 | 96.46 | | | | | 5.33 | 108.01 | 4.20 | 97.13 |
| 4.59 | 100.27 | | | | | 5.35 | 91.72 | 4.20 | 102.84 |
| 4.59 | 96.46 | | | | | 5.39 | 107.60 | 3.84 | 91.71 |
| 4.63 | 101.27 | | | | | | | 3.81 | 91.93 |
| 4.64 | 101.27 | | | | | | | | |
| 4.71 | 101.68 | | | | | | | | |
| 4.72 | 101.74 | | | | | | | | |
| 4.73 | 96.64 | | | | | | | | |
| 4.74 | 96.64 | | | | | | | | |
| 5.06 | 101.39 | | | | | | | | |
| 5.06 | 98.46 | | | | | | | | |
| 5.09 | 96.93 | | | | | | | | |
| 5.12 | 98.05 | | | | | | | | |
| 5.19 | 91.95 | | | | | | | | |
| 5.29 | 97.75 | | | | | | | | |
| 5.30 | 97.75 | | | | | | | | |
| 5.40 | 99.57 | | | | | | | | |
| 5.41 | 97.93 | | | | | | | | |
| 5.41 | 92.13 | | | | | | | | |

| | | |
|---|---|---|
| CLX 115 | CLX 115FC | CLX 109 |

| 1H δ | 13C δ | 1H δ | 13C δ | 1H δ | 13C δ |
|---|---|---|---|---|---|
| [ppm] | [ppm] | [ppm] | [ppm] | [ppm] | [ppm] |
| 4.96 | 100.76 | 5.14 | 99.92 | 4.91 | 92.33 |
| 4.93 | 91.79 | 4.99 | 100.53 | 4.89 | 101.49 |
| 4.86 | 92.07 | 4.96 | 91.54 | 4.85 | 92.53 |
| 4.38 | 103.66 | 4.94 | 101.56 | 4.84 | 106.89 |
| 4.33 | 102.65 | 4.90 | 101.65 | 4.80 | 93.82 |
| 4.32 | 96.44 | 4.89 | 91.89 | 4.75 | 107.74 |
| 4.28 | 102.79 | 4.85 | 92.37 | 4.74 | 94.09 |
| 4.28 | 96.71 | 4.41 | 103.43 | 4.26 | 103.12 |
| 4.27 | 104.10 | 4.36 | 102.43 | 4.24 | 105.38 |
| 4.22 | 96.94 | 4.35 | 96.14 | 4.21 | 102.89 |
| 4.20 | 103.24 | 4.32 | 96.43 | 4.21 | 97.19 |
| | | 4.31 | 102.59 | 4.13 | 103.63 |
| | | 4.26 | 96.68 | | |
| | | 4.23 | 103.00 | | |
| | | 4.20 | 97.14 | | |
| | | 4.16 | 102.93 | | |

TABLE B
CLX101 comprises the oligosaccharides shown in this table. Hex refers
to hexose sugars, Pent refers to pentose sugars, HexA refers to
hexuronic acid sugars, and Deoxyhex refers to deoxyhexose sugars.

| | | | RT | Oligo | Retention |
|---|---|---|---|---|---|
| Compound | Identity | Mass | (min) | Wt. % | Factor |
| 1 | 1Hex1Pent | 312.12 | 1.663 | 0.75 | 1.14 |
| 2 | 2Hex | 342.13 | 1.456 | 3.34 | 1.00 |
| 3 | 2Hex1Pent | 474.17 | 12.672 | 4.45 | 8.70 |
| 4 | 2Hex1Pent | 474.17 | 13.644 | 1.57 | 9.37 |
| 5 | 3Hex | 504.18 | 7.423 | 0.85 | 5.10 |
| 6 | 3Hex | 504.19 | 11.902 | 38.62 | 8.17 |
| 7 | 3Hex1Pent | 636.23 | 24.761 | 0.98 | 17.01 |
| 8 | 4Hex | 666.24 | 24.35 | 20.24 | 16.72 |
| 9 | 5Hex | 828.29 | 30.063 | 18.87 | 20.65 |
| 10 | 6Hex | 990.34 | 36.833 | 10.32 | 25.30 |

TABLE B2
CLX101C comprises the oligosaccharides shown in this
table. Hex refers to hexose sugars, Pent refers
to pentose sugars, HexA refers to hexuronic acid
sugars, and Deoxyhex refers to deoxyhexose sugars

| | | | RT | Oligo | Retention |
|---|---|---|---|---|---|
| Compound | Identity | Mass | (min) | Wt. % | Factor |
| 1 | 2Hex | 342.13 | 1.263 | 0.63 | 1 |
| 2 | 2Hex | 342.13 | 9.312 | 0.53 | 7.373 |
| 3 | 2Hex | 342.13 | 18 | 1.24 | 14.252 |
| 4 | 2Hex1Pent | 474.18 | 1.974 | 0.4 | 1.563 |
| 5 | 2Hex1Pent | 474.18 | 13.842 | 10.3 | 10.96 |
| 6 | 2Hex1Pent | 474.17 | 14.711 | 1.7 | 11.648 |
| 7 | 3Hex | 504.19 | 1.675 | 2.07 | 1.326 |
| 8 | 3Hex | 504.19 | 6.174 | 4.65 | 4.888 |
| 9 | 3Hex | 504.18 | 8.438 | 0.76 | 6.681 |
| 10 | 3Hex | 504.19 | 9 | 4.79 | 7.126 |
| 11 | 3Hex | 504.18 | 12.989 | 1.54 | 10.284 |
| 12 | 3Hex | 504.19 | 13.065 | 28.48 | 10.344 |
| 13 | 3Hex | 504.19 | 13.146 | 1.8 | 10.409 |
| 14 | 3Hex1Pent | 636.23 | 24.812 | 3.2 | 19.645 |
| 15 | 4Hex | 666.24 | 15.164 | 1.84 | 12.006 |
| 16 | 4Hex | 666.24 | 17.989 | 3.07 | 14.243 |
| 17 | 4Hex | 666.24 | 19.593 | 2.58 | 15.513 |
| 18 | 4Hex | 666.24 | 24.196 | 0.81 | 19.158 |
| 19 | 4Hex | 666.24 | 24.28 | 17.2 | 19.224 |
| 20 | 4Hex | 666.24 | 24.364 | 0.82 | 19.291 |
| 21 | 4Hex | 666.24 | 29.72 | 2.01 | 23.531 |
| 22 | 4Hex1Pent | 798.28 | 29.965 | 2.6 | 23.725 |
| 23 | 5Hex | 828.29 | 23.032 | 1.18 | 18.236 |
| 24 | 5Hex | 828.29 | 23.211 | 1.69 | 18.378 |
| 25 | 5Hex | 828.29 | 26.602 | 1.28 | 21.063 |
| 26 | 5Hex | 828.29 | 26.999 | 2.04 | 21.377 |
| 27 | 6Hex | 990.34 | 27.985 | 0.77 | 22.158 |

Table C: CLX102 comprises the oligosaccharides shown
in this table. Hex refers to hexose sugars, Pent refers
to pentose sugars, HexA refers to hexuronic acid sugars,
and Deoxyhex refers to deoxyhexose sugars.

| | | | RT | Oligo | Retention |
|---|---|---|---|---|---|
| Compound | Identity | Mass | (min) | Wt. % | Factor |
| 1 | 2Hex1Pent | 474.17 | 2.263 | 0.17 | 1.518 |
| 2 | 2Hex1Pent | 474.17 | 9.187 | 0.88 | 6.162 |
| 3 | 2Hex1Pent | 474.17 | 10.653 | 0.54 | 7.145 |
| 4 | 2Hex1Pent | 474.17 | 12.713 | 0.37 | 8.526 |
| 5 | 3Hex | 504.18 | 1.491 | 0.84 | 1 |
| 6 | 3Hex | 504.18 | 2.193 | 0.35 | 1.471 |
| 7 | 3Hex | 504.19 | 6.695 | 4.31 | 4.49 |
| 8 | 3Hex | 504.19 | 7.637 | 2.06 | 5.122 |
| 9 | 3Hex | 504.18 | 8.885 | 1.94 | 5.959 |
| 10 | 3Hex | 504.19 | 9.605 | 1.74 | 6.442 |
| 11 | 3Hex | 504.19 | 12.868 | 1.45 | 8.63 |
| 12 | 3Hex | 504.18 | 14.159 | 0.4 | 9.496 |
| 13 | 3Hex1Pent | 636.23 | 6.413 | 0.92 | 4.301 |
| 14 | 3Hex1Pent | 636.23 | 12.074 | 1.13 | 8.098 |
| 15 | 3Hex1Pent | 636.23 | 13.068 | 0.77 | 8.765 |
| 16 | 3Hex1Pent | 636.23 | 18.947 | 1.52 | 12.708 |
| 17 | 3Hex1Pent | 636.23 | 19.573 | 0.75 | 13.127 |
| 18 | 3Hex1Pent | 636.23 | 20.258 | 0.81 | 13.587 |
| 19 | 4Hex | 666.24 | 4.124 | 2.33 | 2.766 |
| 20 | 4Hex | 666.24 | 6.206 | 2.04 | 4.162 |
| 21 | 4Hex | 666.24 | 9.373 | 2.99 | 6.286 |
| 22 | 4Hex | 666.24 | 10.25 | 2.66 | 6.875 |
| 23 | 4Hex | 666.24 | 10.97 | 3.94 | 7.357 |
| 24 | 4Hex | 666.24 | 14.977 | 1.75 | 10.045 |
| 25 | 4Hex | 666.24 | 16.802 | 4.87 | 11.269 |
| 26 | 4Hex | 666.24 | 17.38 | 5.13 | 11.657 |
| 27 | 4Hex | 666.24 | 19.079 | 1.69 | 12.796 |
| 28 | 4Hex | 666.24 | 19.911 | 0.58 | 13.354 |
| 29 | 4Hex | 666.24 | 20.691 | 0.36 | 13.877 |
| 30 | 4Hex | 666.24 | 24.147 | 1.15 | 16.195 |
| 31 | 4Hex1Pent | 798.28 | 9.604 | 0.45 | 6.441 |
| 32 | 4Hex1Pent | 798.28 | 15.28 | 0.48 | 10.248 |
| 33 | 4Hex1Pent | 798.28 | 17.219 | 0.45 | 11.549 |
| 34 | 4Hex1Pent | 798.28 | 18.793 | 0.64 | 12.604 |
| 35 | 4Hex1Pent | 798.28 | 20.328 | 1.41 | 13.634 |
| 36 | 4Hex1Pent | 798.28 | 21.012 | 0.36 | 14.093 |
| 37 | 4Hex1Pent | 798.28 | 21.923 | 0.44 | 14.704 |
| 38 | 4Hex1Pent | 798.28 | 22.714 | 0.55 | 15.234 |
| 39 | 4Hex1Pent | 798.28 | 26.723 | 0.35 | 17.923 |
| 40 | 4Hex1Pent | 798.28 | 27.138 | 0.22 | 18.201 |
| 41 | 4Hex1Pent | 798.28 | 27.347 | 0.26 | 18.341 |
| 42 | 4Hex1Pent | 798.28 | 27.565 | 0.32 | 18.488 |
| 43 | 5Hex | 828.29 | 7.648 | 1.05 | 5.129 |
| 44 | 5Hex | 828.29 | 9.213 | 1.28 | 6.179 |
| 45 | 5Hex | 828.29 | 12.21 | 0.37 | 8.189 |
| 46 | 5Hex | 828.29 | 14.335 | 1.28 | 9.614 |
| 47 | 5Hex | 828.29 | 14.932 | 0.75 | 10.015 |
| 48 | 5Hex | 828.29 | 16.24 | 2.01 | 10.892 |
| 49 | 5Hex | 828.29 | 17.527 | 1.3 | 11.755 |
| 50 | 5Hex | 828.29 | 18.549 | 5.98 | 12.441 |
| 51 | 5Hex | 828.29 | 18.882 | 0.36 | 12.664 |
| 52 | 5Hex | 828.29 | 19.204 | 1.29 | 12.88 |
| 53 | 5Hex | 828.29 | 19.522 | 0.69 | 13.093 |
| 54 | 5Hex | 828.29 | 20.062 | 1.8 | 13.455 |
| 55 | 5Hex | 828.29 | 20.313 | 1.44 | 13.624 |
| 56 | 5Hex | 828.29 | 21.885 | 1.43 | 14.678 |
| 57 | 5Hex | 828.29 | 25.896 | 3.46 | 17.368 |
| 58 | 5Hex | 828.29 | 27.822 | 0.56 | 18.66 |
| 59 | 5Hex | 828.29 | 28.03 | 0.64 | 18.799 |
| 60 | 6Hex | 990.34 | 11.067 | 0.31 | 7.423 |
| 61 | 6Hex | 990.34 | 11.999 | 0.4 | 8.048 |
| 62 | 6Hex | 990.34 | 15.579 | 0.48 | 10.449 |
| 63 | 6Hex | 990.34 | 17.343 | 0.5 | 11.632 |
| 64 | 6Hex | 990.34 | 18.939 | 0.55 | 12.702 |
| 65 | 6Hex | 990.34 | 19.881 | 1.01 | 13.334 |
| 66 | 6Hex | 990.34 | 21.075 | 0.83 | 14.135 |
| 67 | 6Hex | 990.34 | 21.86 | 0.73 | 14.661 |
| 68 | 6Hex | 990.34 | 22.178 | 0.72 | 14.875 |
| 69 | 6Hex | 990.34 | 22.854 | 2.01 | 15.328 |
| 70 | 6Hex | 990.34 | 23.366 | 1.15 | 15.671 |
| 71 | 6Hex | 990.34 | 24.012 | 1.16 | 16.105 |
| 72 | 6Hex | 990.34 | 25.898 | 0.92 | 17.37 |
| 73 | 6Hex | 990.34 | 26.344 | 0.71 | 17.669 |
| 74 | 6Hex | 990.34 | 28.735 | 0.53 | 19.272 |
| 75 | 6Hex | 990.34 | 28.984 | 0.59 | 19.439 |
| 76 | 7Hex | 1152.39 | 17.924 | 0.18 | 12.021 |
| 77 | 7Hex | 1152.4 | 22.096 | 0.58 | 14.82 |
| 78 | 7Hex | 1152.39 | 22.626 | 0.44 | 15.175 |
| 79 | 7Hex | 1152.4 | 22.915 | 0.27 | 15.369 |
| 80 | 7Hex | 1152.39 | 23.686 | 0.42 | 15.886 |
| 81 | 7Hex | 1152.39 | 24.151 | 0.37 | 16.198 |
| 82 | 7Hex | 1152.39 | 24.537 | 0.74 | 16.457 |
| 83 | 7Hex | 1152.39 | 25.769 | 0.59 | 17.283 |
| 84 | 7Hex | 1152.39 | 27.136 | 0.51 | 18.2 |
| 85 | 7Hex | 1152.39 | 28.252 | 0.34 | 18.948 |
| 86 | 7Hex | 1152.4 | 28.633 | 0.57 | 19.204 |
| 87 | 8Hex | 1314.45 | 24.955 | 0.37 | 16.737 |

TABLE D
CLX103 comprises the oligosaccharides shown in this table. Hex refers
to hexose sugars, Pent refers to pentose sugars, HexA refers to
hexuronic acid sugars, and Deoxyhex refers to deoxyhexose sugars.

| | | | RT | Oligo | Retention |
|---|---|---|---|---|---|
| Compound | Identity | Mass | (min) | Wt % | Factor |
| 1 | 2Pent | 282.11 | 6.692 | 1.60 | 1 |
| 2 | 3Pent | 414.16 | 8.429 | 14.82 | 1.26 |
| 3 | 3Pent1HexAOMe | 604.2 | 18.034 | 2.79 | 2.695 |
| 4 | 4Pent | 546.2 | 16.521 | 18.68 | 2.469 |
| 5 | 4Pent1HexAOMe | 736.24 | 20.205 | 2.61 | 3.019 |
| 6 | 4Pent1HexAOMe | 736.24 | 21.15 | 6.91 | 3.16 |
| 7 | 4Pent1HexAOMe | 736.24 | 23.844 | 4.67 | 3.563 |
| 8 | 4Pent1HexAOMe | 736.24 | 25.394 | 2.61 | 3.795 |
| 9 | 4Pent1HexAOMe | 736.24 | 26.435 | 2.91 | 3.95 |
| 10 | 5Hex2Pent | 1092.42 | 8.109 | 1.07 | 1.212 |
| 11 | 5Pent | 678.24 | 8.432 | 1.07 | 1.26 |
| 12 | 5Pent | 678.24 | 23.199 | 19.00 | 3.467 |
| 13 | 6Hex1Pent | 1122.35 | 18.442 | 7.75 | 2.756 |
| 14 | 6Pent | 810.28 | 26.735 | 9.99 | 3.995 |
| 15 | 7Pent | 942.32 | 28.974 | 3.51 | 4.33 |

TABLE E
CLX105 comprises the oligosaccharides shown in this table. Hex refers
to hexose sugars, Pent refers to pentose sugars, HexA refers to
hexuronic acid sugars, and Deoxyhex refers to deoxyhexose sugars.

| | | | RT | | Retention |
|---|---|---|---|---|---|
| Compound | Identity | Mass | (min) | Oligo Wt % | Factor |
| 1 | 2Hex | 342.13 | 1.233 | 0.58 | 1 |
| 2 | 2Hex | 342.13 | 1.43 | 1.79 | 1.16 |
| 3 | 2Hex1Pent | 474.18 | 2.505 | 0.47 | 2.032 |
| 4 | 2Hex1Pent | 474.18 | 3.087 | 0.77 | 2.504 |
| 5 | 2Hex1Pent | 474.18 | 5.487 | 0.38 | 4.45 |
| 6 | 2Hex1Pent | 474.18 | 5.912 | 0.19 | 4.795 |
| 7 | 2Hex1Pent | 474.18 | 6.635 | 0.18 | 5.381 |
| 8 | 2Hex1Pent | 474.18 | 9.208 | 0.61 | 7.468 |
| 9 | 2Hex1Pent | 474.18 | 9.452 | 0.28 | 7.666 |
| 10 | 2Hex2Pent | 606.22 | 3.233 | 0.29 | 2.622 |
| 11 | 2Hex2Pent | 606.22 | 4.847 | 0.75 | 3.931 |
| 12 | 2Hex2Pent | 606.22 | 7.968 | 0.27 | 6.462 |
| 13 | 3Hex | 504.19 | 1.679 | 1.59 | 1.362 |
| 14 | 3Hex | 504.19 | 2.53 | 3.99 | 2.052 |
| 15 | 3Hex | 504.19 | 2.732 | 1.91 | 2.216 |
| 16 | 3Hex | 504.19 | 5.552 | 4.44 | 4.503 |
| 17 | 3Hex1Pent | 636.23 | 6.836 | 0.85 | 5.544 |
| 18 | 3Hex1Pent | 636.23 | 8.019 | 0.25 | 6.504 |
| 19 | 3Hex1Pent | 636.23 | 9.729 | 0.23 | 7.891 |
| 20 | 3Hex1Pent | 636.23 | 9.949 | 0.63 | 8.069 |
| 21 | 3Hex1Pent | 636.23 | 11.439 | 1.27 | 9.277 |
| 22 | 3Hex2Pent | 768.27 | 8.577 | 0.20 | 6.956 |
| 23 | 3Hex3Pent | 900.31 | 12.393 | 0.29 | 10.051 |
| 24 | 4Hex | 666.24 | 3.534 | 6.47 | 2.866 |
| 25 | 4Hex | 666.24 | 4.2 | 5.10 | 3.406 |
| 26 | 4Hex | 666.24 | 5.381 | 4.02 | 4.364 |
| 27 | 4Hex | 666.24 | 6.643 | 5.20 | 5.388 |
| 28 | 4Hex | 666.24 | 8.843 | 7.15 | 7.172 |
| 29 | 4Hex | 666.24 | 9.955 | 2.70 | 8.074 |
| 30 | 4Hex | 666.24 | 11.036 | 0.91 | 8.951 |
| 31 | 4Hex | 666.24 | 14.586 | 1.15 | 11.83 |
| 32 | 4Hex1Pent | 798.28 | 12.257 | 0.21 | 9.941 |
| 33 | 4Hex1Pent | 798.28 | 12.449 | 0.23 | 10.097 |
| 34 | 4Hex1Pent | 798.28 | 12.783 | 0.63 | 10.367 |
| 35 | 4Hex1Pent | 798.28 | 14.336 | 0.50 | 11.627 |
| 36 | 4Hex1Pent | 798.28 | 18.353 | 0.52 | 14.885 |
| 37 | 5Hex | 828.29 | 6.433 | 2.57 | 5.217 |
| 38 | 5Hex | 828.29 | 6.983 | 2.91 | 5.663 |
| 39 | 5Hex | 828.29 | 9.107 | 2.45 | 7.386 |
| 40 | 5Hex | 828.29 | 10.555 | 4.78 | 8.56 |
| 41 | 5Hex | 828.29 | 11.023 | 2.10 | 8.94 |
| 42 | 5Hex | 828.29 | 11.7 | 5.43 | 9.489 |
| 43 | 5Hex | 828.29 | 14.088 | 0.77 | 11.426 |
| 44 | 5Hex | 828.29 | 15.047 | 3.80 | 12.204 |
| 45 | 6Hex | 990.34 | 8.723 | 0.94 | 7.075 |
| 46 | 6Hex | 990.34 | 11.052 | 0.76 | 8.964 |
| 47 | 6Hex | 990.35 | 11.731 | 1.68 | 9.514 |
| 48 | 6Hex | 990.34 | 11.938 | 1.53 | 9.682 |
| 49 | 6Hex | 990.34 | 12.269 | 2.86 | 9.951 |
| 50 | 6Hex | 990.34 | 14.213 | 0.73 | 11.527 |
| 51 | 6Hex | 990.35 | 14.432 | 0.24 | 11.705 |
| 52 | 6Hex | 990.35 | 15.11 | 1.94 | 12.255 |
| 53 | 6Hex | 990.34 | 16.676 | 1.33 | 13.525 |
| 54 | 6Hex | 990.35 | 17.193 | 0.92 | 13.944 |
| 55 | 6Hex | 990.34 | 17.84 | 0.56 | 14.469 |
| 56 | 6Hex | 990.34 | 21.269 | 1.03 | 17.25 |
| 57 | 7Hex | 1152.4 | 12.943 | 0.51 | 10.497 |
| 58 | 7Hex | 1152.39 | 13.193 | 0.47 | 10.7 |
| 59 | 7Hex | 1152.39 | 13.639 | 0.53 | 11.062 |
| 60 | 7Hex | 1152.4 | 16.038 | 0.26 | 13.007 |
| 61 | 7Hex | 1152.4 | 16.731 | 0.50 | 13.569 |
| 62 | 7Hex | 1152.4 | 17.67 | 1.40 | 14.331 |

TABLE F
CLX108 comprises the oligosaccharides shown in this table. Hex refers
to hexose sugars, Pent refers to pentose sugars, HexA refers to
hexuronic acid sugars, and Deoxyhex refers to deoxyhexose sugars.

| Com- | | | RT | | Retention |
|---|---|---|---|---|---|
| pound | Identity | Mass | (min) | Oligos Wt % | Factor |
| 1 | 3Hex | 504.19 | 1.492 | 2.27 | 1 |
| 2 | 3Hex | 504.19 | 1.855 | 0.85 | 1.243 |
| 3 | 3Hex | 504.18 | 3.002 | 0.75 | 2.012 |
| 4 | 3Hex | 504.18 | 4.461 | 1.28 | 2.99 |
| 5 | 3Hex (nr) | 504.17 | 11.02 | 9.48 | 7.386 |
| 6 | 3Hex1Pent | 636.23 | 6.511 | 4.42 | 4.364 |
| 7 | 3Hex1Pent | 636.23 | 7.066 | 2.09 | 4.736 |
| 8 | 3Hex1Pent | 636.23 | 8.646 | 1.15 | 5.795 |
| 9 | 4Hex | 666.24 | 4.188 | 7.94 | 2.807 |
| 10 | 4Hex | 666.24 | 4.911 | 2.47 | 3.292 |
| 11 | 4Hex | 666.24 | 5.225 | 3.70 | 3.502 |
| 12 | 4Hex | 666.24 | 5.608 | 3.28 | 3.759 |
| 13 | 4Hex | 666.24 | 7.33 | 1.86 | 4.913 |
| 14 | 4Hex | 666.24 | 15.185 | 1.00 | 10.178 |
| 15 | 4Hex1Deoxyhex | 812.26 | 10.546 | 0.99 | 7.068 |
| 16 | 4Hex1HexA | 842.27 | 10.31 | 2.49 | 6.91 |
| 17 | 4Hex1Pent | 798.28 | 9.688 | 4.29 | 6.493 |
| 18 | 4Hex1Pent | 798.28 | 11.09 | 2.19 | 7.433 |
| 19 | 4Hex1Pent | 798.28 | 11.592 | 1.11 | 7.769 |
| 20 | 4Hex1Pent | 798.28 | 14.207 | 0.98 | 9.522 |
| 21 | 5Hex | 828.29 | 7.755 | 6.79 | 5.198 |
| 22 | 5Hex | 828.29 | 7.992 | 2.61 | 5.357 |
| 23 | 5Hex | 828.29 | 8.599 | 2.52 | 5.763 |
| 24 | 5Hex | 828.29 | 9.09 | 2.45 | 6.092 |
| 25 | 5Hex | 828.29 | 9.802 | 1.88 | 6.57 |
| 26 | 5Hex | 828.29 | 10.062 | 3.12 | 6.744 |
| 27 | 5Hex | 828.29 | 13.089 | 2.17 | 8.773 |
| 28 | 6Hex | 990.34 | 11.153 | 5.21 | 7.475 |
| 29 | 6Hex | 990.34 | 12.344 | 1.34 | |
| 30 | 5Hex | 828.29 | 13.089 | 2.17 | |
| 31 | 7Hex | 1152.39 | 13.293 | 3.21 | |
| 32 | 6Hex | 990.34 | 13.877 | 2.18 | |
| 33 | 7Hex | 1152.39 | 14.078 | 1.24 | |
| 34 | 4Hex1Pent | 798.28 | 14.207 | 0.98 | |
| 35 | 6Hex | 990.34 | 14.341 | 2.19 | |
| 36 | 4Hex | 666.24 | 15.185 | 1.00 | |
| 37 | 6Hex | 990.34 | 15.221 | 1.50 | |
| 38 | 7Hex | 1152.39 | 15.273 | 1.03 | |
| 39 | 7Hex | 1152.39 | 16.279 | 1.25 | |

TABLE G
CLX109 comprises the oligosaccharides shown in this table. Hex refers
to hexose sugars, Pent refers to pentose sugars, HexA refers to
hexuronic acid sugars, and Deoxyhex refers to deoxyhexose sugars.

| Com- | | | RT | | Retention |
|---|---|---|---|---|---|
| pound | Identity | Mass | (min) | Oligo Wt % | Factor |
| 1 | 2Hex1Pent | 474.18 | 2.658 | 0.59 | 1 |
| 2 | 2Hex1Pent | 474.17 | 3.038 | 5.45 | 1.143 |
| 3 | 3Hex | 504.19 | 2.69 | 6.74 | 1.012 |
| 4 | 3Hex1HexA | 680.22 | 8.937 | 9.12 | 3.362 |
| 5 | 3Hex1Pent | 636.23 | 6.014 | 0.60 | 2.263 |
| 6 | 3Hex1Pent | 636.23 | 7.292 | 10.79 | 2.743 |
| 7 | 4Hex | 666.24 | 6.614 | 15.53 | 2.488 |
| 8 | 4Hex1Deoxyhex | 812.26 | 13.126 | 3.46 | 4.938 |
| 9 | 4Hex1HexA | 842.27 | 11.857 | 11.47 | 4.461 |
| 10 | 4Hex1Pent | 798.28 | 9.508 | 0.62 | 3.577 |
| 11 | 4Hex1Pent | 798.28 | 10.112 | 7.58 | 3.804 |
| 12 | 5Hex | 828.29 | 9.652 | 9.27 | 3.631 |
| 13 | 5Hex1Deoxyhex | 974.31 | 14.753 | 2.85 | 5.55 |
| 14 | 5Hex1HexA | 1004.32 | 13.573 | 7.20 | 5.106 |
| 15 | 6Hex | 990.34 | 11.515 | 4.50 | 4.332 |
| 16 | 6Hex1HexA | 1166.37 | 14.673 | 3.11 | 5.52 |
| 17 | 7Hex | 1152.39 | 12.782 | 1.15 | 4.809 |

TABLE H
CLX110 comprises the oligosaccharides shown in this table. Hex refers
to hexose sugars, Pent refers to pentose sugars, HexA refers to
hexuronic acid sugars, and Deoxyhex refers to deoxyhexose sugars.

| Com- | | | RT | | Retention |
|---|---|---|---|---|---|
| pound | Identity | Mass | (min) | Oligo Wt % | Factor |
| 1 | 1Hex2Pent | 444.17 | 2.988 | 0.50 | 2.008 |
| 2 | 2Hex | 342.13 | 2.925 | 0.46 | 1.966 |
| 3 | 2Hex1Pent | 474.18 | 2.211 | 0.62 | 1.486 |
| 4 | 2Hex2Pent | 606.22 | 8.398 | 2.08 | 5.644 |
| 5 | 3Hex | 504.19 | 1.488 | 0.99 | 1 |
| 6 | 3Hex | 504.18 | 6.593 | 0.81 | 4.431 |
| 7 | 3Hex1Deoxyhex | 650.24 | 5.754 | 0.30 | 3.867 |
| 8 | 3Hex1HexA | 680.22 | 5.977 | 1.65 | 4.017 |
| 9 | 3Hex1Pent | 636.23 | 6.275 | 7.59 | 4.217 |
| 10 | 3Hex1Pent | 636.23 | 6.83 | 1.29 | 4.59 |
| 11 | 3Hex1Pent | 636.23 | 8.396 | 0.29 | 5.642 |
| 12 | 3Hex2Pent | 768.27 | 10.68 | 1.43 | 7.177 |
| 13 | 4Hex | 666.24 | 4.016 | 17.74 | 2.699 |
| 14 | 4Hex | 666.24 | 4.726 | 0.99 | 3.176 |
| 15 | 4Hex | 666.24 | 5.06 | 0.58 | 3.401 |
| 16 | 4Hex | 666.24 | 5.439 | 0.93 | 3.655 |
| 17 | 4Hex | 666.24 | 9.279 | 0.88 | 6.236 |
| 18 | 4Hex | 666.24 | 10.087 | 0.35 | 6.779 |
| 19 | 4Hex | 666.24 | 16.429 | 1.54 | 11.041 |
| 20 | 4Hex1Deoxyhex | 812.26 | 10.22 | 0.78 | 6.868 |
| 21 | 4Hex1HexA | 842.27 | 9.907 | 4.28 | 6.658 |
| 22 | 4Hex1HexA | 842.27 | 10.504 | 0.59 | 7.059 |
| 23 | 4Hex1Pent | 798.28 | 9.459 | 6.96 | 6.357 |
| 24 | 5Hex | 828.29 | 7.567 | 15.88 | 5.085 |
| 25 | 5Hex | 828.29 | 8.882 | 0.70 | 5.969 |
| 26 | 5Hex | 828.29 | 9.089 | 0.61 | 6.108 |
| 27 | 5Hex | 828.29 | 9.783 | 1.38 | 6.575 |
| 28 | 5Hex | 828.29 | 17.217 | 0.47 | 11.571 |
| 29 | 5Hex | 828.29 | 18.075 | 0.52 | 12.147 |
| 30 | 5Hex1Deoxyhex | 974.31 | 13.623 | 0.88 | 9.155 |
| 31 | 5Hex1HexA | 1004.33 | 12.747 | 4.44 | 8.567 |
| 32 | 5Hex1HexA | 1004.32 | 13.358 | 0.79 | 8.977 |
| 33 | 5Hex1Pent | 960.34 | 12.139 | 2.06 | 8.158 |
| 34 | 6Hex | 990.35 | 10.877 | 8.71 | 7.31 |
| 35 | 6Hex | 990.35 | 11.152 | 0.34 | 7.495 |
| 36 | 6Hex | 990.34 | 11.405 | 0.55 | 7.665 |
| 37 | 6Hex | 990.35 | 11.76 | 0.64 | 7.903 |
| 38 | 6Hex | 990.34 | 14.094 | 0.59 | 9.472 |
| 39 | 6Hex1HexA | 1166.38 | 15.181 | 1.68 | 10.202 |
| 40 | 6Hex1Pent | 1122.39 | 14.439 | 0.96 | 9.704 |
| 41 | 7Hex | 1152.4 | 13.039 | 4.18 | 8.763 |
| 42 | 8Hex | 1314.45 | 14.906 | 0.98 | 10.017 |

TABLE I
CLX111 comprises the oligosaccharides shown in this table. Hex refers
to hexose sugars, Pent refers to pentose sugars, HexA refers to
hexuronic acid sugars, and Deoxyhex refers to deoxyhexose sugars.

| Com- | | | RT | | Retention |
|---|---|---|---|---|---|
| pound | Identity | Mass | (min) | OligoWt % | Factor |
| 1 | 3Hex | 504.19 | 1.489 | 2.72 | 1 |
| 2 | 3Hex | 504.19 | 1.845 | 1.04 | 1.2391 |
| 3 | 3Hex | 504.19 | 2.978 | 1.54 | 2 |
| 4 | 3Hex1Pent | 636.23 | 6.404 | 1.79 | 4.3009 |
| 5 | 3Hex1Pent | 636.23 | 6.968 | 1.16 | 4.6797 |
| 6 | 3Hex1Pent | 636.23 | 8.198 | 0.38 | 5.5057 |
| 7 | 3Hex1Pent | 636.23 | 8.566 | 0.55 | 5.7529 |
| 8 | 3Hex2Pent | 768.27 | 10.827 | 0.28 | 7.2713 |
| 9 | 3Hex2Pent | 768.27 | 12.342 | 0.23 | 8.2888 |
| 10 | 3Hex2Pent | 768.27 | 13.372 | 0.43 | 8.9805 |
| 11 | 4Hex | 666.24 | 4.109 | 12.41 | 2.7596 |
| 12 | 4Hex | 666.24 | 4.802 | 4.74 | 3.225 |
| 13 | 4Hex | 666.24 | 5.122 | 5.89 | 3.4399 |
| 14 | 4Hex | 666.24 | 5.502 | 6.4 | 3.6951 |
| 15 | 4Hex | 666.24 | 7.239 | 3.54 | 4.8617 |
| 16 | 4Hex1Deoxyhex | 812.26 | 10.495 | 0.58 | 7.0484 |
| 17 | 4Hex1Deoxyhex | 812.26 | 14.627 | 0.42 | 9.8234 |
| 18 | 4Hex1HexA | 842.27 | 9.515 | 0.39 | 6.3902 |
| 19 | 4Hex1HexA | 842.27 | 10.301 | 1.26 | 6.9181 |
| 20 | 4Hex1HexA | 842.27 | 11.02 | 0.71 | 7.4009 |
| 21 | 4Hex1HexA | 842.27 | 11.725 | 0.52 | 7.8744 |
| 22 | 4Hex1HexA | 842.27 | 12.33 | 0.98 | 8.2807 |
| 23 | 4Hex1HexA | 842.27 | 14.866 | 0.43 | 9.9839 |
| 24 | 4Hex1HexA | 842.27 | 16.706 | 0.33 | 11.2196 |
| 25 | 4Hex1Pent | 798.28 | 9.614 | 1.3 | 6.4567 |
| 26 | 4Hex1Pent | 798.28 | 10.996 | 0.83 | 7.3848 |
| 27 | 4Hex1Pent | 798.28 | 11.525 | 0.31 | 7.7401 |
| 28 | 4Hex1Pent | 798.28 | 14.191 | 0.44 | 9.5306 |
| 29 | 5Hex | 828.29 | 7.65 | 7.46 | 5.1377 |
| 30 | 5Hex | 828.29 | 7.896 | 3.61 | 5.3029 |
| 31 | 5Hex | 828.29 | 8.52 | 2.77 | 5.722 |
| 32 | 5Hex | 828.29 | 9.013 | 3.16 | 6.0531 |
| 33 | 5Hex | 828.29 | 9.737 | 2.53 | 6.5393 |
| 34 | 5Hex | 828.29 | 9.999 | 4.5 | 6.7152 |
| 35 | 5Hex | 828.29 | 11.477 | 0.62 | 7.7079 |
| 36 | 5Hex | 828.29 | 13.038 | 3.41 | 8.7562 |
| 37 | 6Hex | 990.34 | 11.077 | 3.58 | 7.4392 |
| 38 | 6Hex | 990.34 | 11.342 | 1.11 | 7.6172 |
| 39 | 6Hex | 990.34 | 11.581 | 1.58 | 7.7777 |
| 40 | 6Hex | 990.34 | 11.96 | 1.27 | 8.0322 |
| 41 | 6Hex | 990.34 | 12.288 | 1.28 | 8.2525 |
| 42 | 6Hex | 990.34 | 12.746 | 0.79 | 8.5601 |
| 43 | 6Hex | 990.34 | 13.842 | 1.74 | 9.2962 |
| 44 | 6Hex | 990.34 | 14.332 | 2.32 | 9.6253 |
| 45 | 6Hex | 990.34 | 15.227 | 1.21 | 10.2263 |
| 46 | 6Hex | 990.34 | 17.443 | 0.31 | 11.7146 |
| 47 | 7Hex | 1152.4 | 13.245 | 1.35 | 8.8952 |
| 48 | 7Hex | 1152.4 | 14.057 | 0.86 | 9.4406 |
| 49 | 7Hex | 1152.39 | 14.302 | 0.48 | 9.6051 |
| 50 | 7Hex | 1152.39 | 14.551 | 0.39 | 9.7723 |
| 51 | 7Hex | 1152.4 | 15.271 | 0.49 | 10.2559 |
| 52 | 7Hex | 1152.39 | 15.516 | 0.31 | 10.4204 |
| 53 | 7Hex | 1152.4 | 16.316 | 0.79 | 10.9577 |
| 54 | 7Hex | 1152.39 | 17.823 | 0.51 | 11.9698 |

TABLE J
CLX112 comprises the oligosaccharides shown in this table. Hex refers
to hexose sugars, Pent refers to pentose sugars, HexA refers to
hexuronic acid sugars, and Deoxyhex refers to deoxyhexose sugars.

| Com- | | | RT | | |
|---|---|---|---|---|---|
| pound | Identity | Mass | (min) | Oligo Wt % | Retention Factor |
| 1 | 3Hex | 504.19 | 10.431 | 7.9 | 1 |
| 2 | 3Hex | 504.19 | 14.158 | 13.53 | 1.357 |
| 3 | 3Hex | 504.19 | 17.074 | 5.47 | 1.637 |
| 4 | 4Hex | 666.24 | 21.248 | 11.27 | 2.037 |
| 5 | 4Hex | 666.24 | 24.78 | 6.29 | 2.376 |
| 6 | 4Hex | 666.24 | 25.028 | 8.82 | 2.399 |
| 7 | 4Hex | 666.24 | 25.857 | 9.81 | 2.479 |
| 8 | 5Hex | 828.29 | 27.544 | 2.08 | 2.641 |
| 9 | 5Hex | 828.29 | 28.172 | 3.8 | 2.701 |
| 10 | 5Hex | 828.29 | 28.914 | 1.18 | 2.772 |
| 11 | 5Hex | 828.29 | 29.507 | 7.33 | 2.829 |
| 12 | 5Hex | 828.29 | 30.241 | 11.24 | 2.899 |
| 13 | 6Hex | 990.34 | 32.369 | 2.62 | 3.103 |
| 14 | 6Hex | 990.34 | 34.032 | 6.74 | 3.263 |
| 15 | 6Hex | 990.34 | 37.05 | 1.95 | 3.552 |

TABLE K
CLX113 comprises the oligosaccharides shown in this table. Hex refers
to hexose sugars, Pent refers to pentose sugars, HexA refers to
hexuronic acid sugars, and Deoxyhex refers to deoxyhexose sugars.

| Com- | | | RT | | Retention |
|---|---|---|---|---|---|
| pound | Identity | Mass | (min) | Oligo Wt % | Factor |
| 1 | 2Hex1Pent | 474.17 | 6.596 | 8.71 | 1 |
| 2 | 2Hex1Pent | 474.17 | 7.182 | 1.54 | 1.089 |
| 3 | 2Hex1Pent | 474.17 | 9.915 | 6.7 | 1.503 |
| 4 | 2Hex2Pent | 606.22 | 10.489 | 0.41 | 1.59 |
| 5 | 2Hex2Pent | 606.22 | 14.055 | 8.77 | 2.131 |
| 6 | 3Hex1Pent | 636.23 | 11.569 | 2.3 | 1.754 |
| 7 | 3Hex1Pent | 636.23 | 12.735 | 4.64 | 1.931 |
| 8 | 3Hex1Pent | 636.23 | 17.858 | 2.32 | 2.707 |
| 9 | 3Hex1Pent | 636.23 | 19.639 | 2.6 | 2.977 |
| 10 | 3Hex1Pent | 636.23 | 21.047 | 2.57 | 3.191 |
| 11 | 3Hex2Pent | 768.27 | 16.236 | 1.77 | 2.461 |
| 12 | 3Hex2Pent | 768.27 | 16.913 | 7.56 | 2.564 |
| 13 | 3Hex2Pent | 768.27 | 22.6 | 4.71 | 3.426 |
| 14 | 3Hex2Pent | 768.27 | 23.712 | 4.79 | 3.595 |
| 15 | 3Hex2Pent | 768.27 | 24.427 | 2.85 | 3.703 |
| 16 | 3Hex3Pent | 900.31 | 25.675 | 1.94 | 3.893 |
| 17 | 4Hex1Pent | 798.28 | 19.976 | 2.44 | 3.029 |
| 18 | 4Hex1Pent | 798.28 | 22.29 | 1.57 | 3.379 |
| 19 | 4Hex1Pent | 798.28 | 26.972 | 0.62 | 4.089 |
| 20 | 4Hex1Pent | 798.28 | 27.808 | 0.91 | 4.216 |
| 21 | 4Hex1Pent | 798.28 | 28.061 | 0.79 | 4.254 |
| 22 | 4Hex2Pent | 930.32 | 18.582 | 2.65 | 2.817 |
| 23 | 4Hex2Pent | 930.32 | 22.74 | 1.7 | 3.448 |
| 24 | 4Hex2Pent | 930.32 | 23.905 | 1.69 | 3.624 |
| 25 | 4Hex2Pent | 930.32 | 24.564 | 3.31 | 3.724 |
| 26 | 4Hex2Pent | 930.32 | 24.966 | 3.86 | 3.785 |
| 27 | 4Hex2Pent | 930.32 | 27.736 | 1.18 | 4.205 |
| 28 | 4Hex2Pent | 930.32 | 28.44 | 0.98 | 4.312 |
| 29 | 4Hex2Pent | 930.32 | 28.778 | 1.97 | 4.363 |
| 30 | 4Hex2Pent | 930.32 | 29.18 | 1.24 | 4.424 |
| 31 | 4Hex2Pent | 930.32 | 30.836 | 0.75 | 4.675 |
| 32 | 4Hex3Pent | 1062.36 | 26.017 | 1.49 | 3.944 |
| 33 | 4Hex3Pent | 1062.36 | 26.225 | 1.2 | 3.976 |
| 34 | 4Hex3Pent | 1062.36 | 29.365 | 0.99 | 4.452 |
| 35 | 4Hex3Pent | 1062.36 | 29.76 | 0.96 | 4.512 |
| 36 | 4Hex3Pent | 1062.36 | 31.172 | 0.58 | 4.726 |
| 37 | 4Hex3Pent | 1062.36 | 31.49 | 1.02 | 4.774 |
| 38 | 5Hex3Pent | 1224.42 | 26.484 | 1.27 | 4.015 |
| 39 | 5Hex3Pent | 1224.42 | 28.91 | 0.54 | 4.383 |
| 40 | 5Hex3Pent | 1224.42 | 29.282 | 0.74 | 4.439 |
| 41 | 5Hex3Pent | 1224.42 | 30.009 | 0.89 | 4.55 |
| 42 | 5Hex3Pent | 1224.42 | 31.349 | 0.47 | 4.753 |

TABLE L
CLX114 comprises the oligosaccharides shown in this table. Hex refers
to hexose sugars, Pent refers to pentose sugars, HexA refers to
hexuronic acid sugars, and Deoxyhex refers to deoxyhexose sugars.

| Com- | | | RT | Oligo Wt | Retention |
|---|---|---|---|---|---|
| pound | Identity | Mass | (min) | % | Factor |
| 1 | 3Pent | 414.15 | 5.215 | 2.15 | 1 |
| 2 | 3Pent | 414.15 | 8.612 | 5.60 | 1.651 |
| 3 | 3Pent | 414.15 | 11.556 | 5.01 | 2.216 |
| 4 | 3Pent | 414.15 | 14.346 | 8.99 | 2.751 |
| 5 | 3Pent | 414.15 | 21.696 | 3.32 | 4.16 |
| 6 | 4Pent | 546.2 | 7.539 | 2.29 | 1.446 |
| 7 | 4Pent | 546.2 | 8.925 | 5.02 | 1.711 |
| 8 | 4Pent | 546.19 | 16.952 | 4.99 | 3.251 |
| 9 | 4Pent | 546.2 | 20.455 | 5.68 | 3.922 |
| 10 | 5Pent | 678.24 | 11.806 | 3.97 | 2.264 |
| 11 | 5Pent | 678.24 | 16.229 | 4.00 | 3.112 |
| 12 | 5Pent | 678.24 | 19.91 | 5.20 | 3.818 |
| 13 | 5Pent | 678.24 | 20.812 | 8.23 | 3.991 |
| 14 | 5Pent | 678.24 | 23.691 | 3.63 | 4.543 |
| 15 | 5Pent | 678.24 | 25.947 | 6.89 | 4.975 |
| 16 | 6Pent | 810.28 | 17.177 | 2.16 | 3.294 |
| 17 | 6Pent | 810.28 | 21.326 | 2.88 | 4.089 |
| 18 | 6Pent | 810.28 | 24.969 | 4.62 | 4.788 |
| 19 | 6Pent | 810.28 | 25.26 | 3.51 | 4.844 |
| 20 | 6Pent | 810.28 | 26.913 | 3.30 | 5.161 |
| 21 | 7Pent | 942.32 | 25.618 | 2.90 | 4.912 |
| 22 | 7Pent | 942.32 | 27.697 | 5.66 | 5.311 |

TABLE M
CLX115 comprises the oligosaccharides shown in this table. Hex refers
to hexose sugars, Pent refers to pentose sugars, HexA refers to
hexuronic acid sugars, and Deoxyhex refers to deoxyhexose sugars.

| Com- | | | | Oligos | Retention |
|---|---|---|---|---|---|
| pound | Identity | Mass | RT (min) | Wt % | Factor |
| 1 | 1Hex1Pent | 314.1214 | 1.839 | 0.09 | 1.196 |
| 2 | 1Hex2Pent | 446.1638 | 14.618 | 0.46 | 9.511 |
| 3 | 1Hex2Pent | 446.1641 | 14.73 | 0.17 | 9.584 |
| 4 | 1Hex2Pent | 446.1635 | 15.648 | 0.28 | 10.181 |
| 5 | 1Hex2Pent | 446.1643 | 17.222 | 0.11 | 11.205 |
| 6 | 2Hex | 344.1321 | 1.537 | 1.05 | 1 |
| 7 | 2Hex | 344.1329 | 2.878 | 2.41 | 1.872 |
| 8 | 2Hex1Pent | 476.1744 | 12.931 | 1.06 | 8.413 |
| 9 | 2Hex1Pent | 476.174 | 13.229 | 0.49 | 8.607 |
| 10 | 2Hex1Pent | 476.1753 | 13.732 | 1.53 | 8.934 |
| 11 | 2Hex1Pent | 476.1742 | 15.112 | 0.33 | 9.832 |
| 12 | 2Hex1Pent | 476.1748 | 16.113 | 0.84 | 10.483 |
| 13 | 2Hex1Pent | 476.1742 | 16.527 | 0.33 | 10.753 |
| 14 | 2Hex2Pent | 608.2157 | 24.944 | 0.45 | 16.229 |
| 15 | 2Hex2Pent | 608.2161 | 26.779 | 0.19 | 17.423 |
| 16 | 3Hex | 506.1856 | 3.295 | 0.63 | 2.144 |
| 17 | 3Hex | 506.1856 | 4.549 | 0.8 | 2.96 |
| 18 | 3Hex | 506.1843 | 10.5 | 0.08 | 6.831 |
| 19 | 3Hex | 506.1855 | 11.335 | 7.55 | 7.375 |
| 20 | 3Hex | 506.1855 | 14.01 | 0.23 | 9.115 |
| 21 | 3Hex | 506.1857 | 15.193 | 9.74 | 9.885 |
| 22 | 3Hex | 506.1857 | 15.445 | 0.05 | 10.049 |
| 23 | 3Hex | 506.184 | 15.874 | 0.08 | 10.328 |
| 24 | 3Hex | 506.1856 | 17.831 | 6.41 | 11.601 |
| 25 | 3Hex1HexA | 682.2171 | 25.124 | 0.13 | 16.346 |
| 26 | 3Hex1HexA | 682.2176 | 27.182 | 0.58 | 17.685 |
| 27 | 3Hex1HexA | 682.2177 | 27.272 | 0.19 | 17.744 |
| 28 | 3Hex1HexA | 682.2169 | 28.058 | 0.67 | 18.255 |
| 29 | 3Hex1Pent | 638.2261 | 9.335 | 0.1 | 6.074 |
| 30 | 3Hex1Pent | 638.2263 | 10.131 | 0.07 | 6.591 |
| 31 | 3Hex1Pent | 638.2276 | 23.161 | 0.93 | 15.069 |
| 32 | 3Hex1Pent | 638.2275 | 23.637 | 0.4 | 15.379 |
| 33 | 3HexlPent | 638.2273 | 24.298 | 0.44 | 15.809 |
| 34 | 3Hex1Pent | 638.2271 | 25.161 | 0.72 | 16.37 |
| 35 | 3Hex1Pent | 638.2282 | 25.768 | 0.84 | 16.765 |
| 36 | 3Hex1Pent | 638.2271 | 26.311 | 0.23 | 17.118 |
| 37 | 3Hex2Pent | 770.2696 | 29.593 | 0.06 | 19.254 |
| 38 | 3Hex2Pent | 770.2686 | 30.152 | 0.19 | 19.617 |
| 39 | 3Hex2Pent | 770.2681 | 30.838 | 0.07 | 20.064 |
| 40 | 3Hex2Pent | 770.2695 | 30.903 | 0.12 | 20.106 |
| 41 | 4Hex | 668.237 | 7.62 | 0.19 | 4.958 |
| 42 | 4Hex | 668.238 | 8.72 | 0.1 | 5.673 |
| 43 | 4Hex | 668.237 | 9.276 | 1.35 | 6.035 |
| 44 | 4Hex | 668.2351 | 9.819 | 0.06 | 6.388 |
| 45 | 4Hex | 668.2378 | 10.269 | 1.06 | 6.681 |
| 46 | 4Hex | 668.2379 | 10.63 | 0.34 | 6.916 |
| 47 | 4Hex | 668.2388 | 21.557 | 8.22 | 14.025 |
| 48 | 4Hex | 668.2386 | 24.211 | 0.1 | 15.752 |
| 49 | 4Hex | 668.239 | 25.244 | 4.57 | 16.424 |
| 50 | 4Hex | 668.239 | 25.507 | 5.92 | 16.595 |
| 51 | 4Hex | 668.2387 | 26.403 | 6.18 | 17.178 |
| 52 | 4Hex | 668.2343 | 26.607 | 0.04 | 17.311 |
| 53 | 4Hex1Deoxyhex | 814.2585 | 31.081 | 0.31 | 20.222 |
| 54 | 4Hex1HexA | 844.247 | 29.807 | 0.04 | 19.393 |
| 55 | 4Hex1Pent | 800.2787 | 28.827 | 0.23 | 18.755 |
| 56 | 4Hex1Pent | 800.2797 | 29.062 | 0.41 | 18.908 |
| 57 | 4Hex1Pent | 800.2787 | 29.552 | 0.11 | 19.227 |
| 58 | 4Hex1Pent | 800.2799 | 29.615 | 0.56 | 19.268 |
| 59 | 4Hex1Pent | 800.2788 | 30.227 | 1.2 | 19.666 |
| 60 | 5Hex | 830.2911 | 11.751 | 0.2 | 7.645 |
| 61 | 5Hex | 830.2895 | 13.261 | 0.48 | 8.628 |
| 62 | 5Hex | 830.2893 | 13.518 | 0.46 | 8.795 |
| 63 | 5Hex | 830.2899 | 13.84 | 0.71 | 9.005 |
| 64 | 5Hex | 830.2907 | 27.932 | 2.29 | 18.173 |
| 65 | 5Hex | 830.2922 | 28.423 | 3.57 | 18.493 |
| 66 | 5Hex | 830.2907 | 29.322 | 1.02 | 19.077 |
| 67 | 5Hex | 830.2908 | 29.82 | 4.98 | 19.401 |
| 68 | 5Hex | 830.291 | 30.469 | 6.13 | 19.824 |
| 69 | 6Hex | 992.3433 | 14.683 | 0.1 | 9.553 |
| 70 | 6Hex | 992.342 | 15.905 | 0.98 | 10.348 |
| 71 | 6Hex | 992.3433 | 31.896 | 1.52 | 20.752 |
| 72 | 6Hex | 992.3427 | 31.937 | 1.02 | 20.779 |
| 73 | 6Hex | 992.3434 | 33.337 | 0.52 | 21.69 |
| 74 | 6Hex | 992.3429 | 33.37 | 1.9 | 21.711 |
| 75 | 6Hex | 992.3429 | 33.763 | 0.91 | 21.967 |
| 76 | 6Hex | 992.3421 | 35.487 | 1.12 | 23.088 |

TABLE M2
CLX115FC comprises the oligosaccharides shown in this
table. Hex refers to hexose sugars, Pent refers to
pentose sugars, HexA refers to hexuronic acid sugars,
and Deoxyhex refers to deoxyhexose sugars.

| Com- | | | RT | | Retention |
|---|---|---|---|---|---|
| pound | Identity | Mass | (min) | Oligo Wt. % | Factor |
| 1 | 1Hex1Pent | 312.12 | 2.345 | 0.66 | 1.364 |
| 2 | 1Hex2Pent | 444.16 | 15.86 | 0.29 | 9.226 |
| 3 | 2Hex | 342.13 | 1.719 | 0.87 | 1 |
| 4 | 2Hex | 342.13 | 3.253 | 2.97 | 1.892 |
| 5 | 2Hex | 342.13 | 3.843 | 0.17 | 2.236 |
| 6 | 2Hex1Pent | 474.17 | 13.261 | 0.74 | 7.714 |
| 7 | 2Hex1Pent | 474.17 | 14.03 | 1.68 | 8.162 |
| 8 | 2Hex1Pent | 474.17 | 16.297 | 1.08 | 9.481 |
| 9 | 2Hex1Pent | 474.17 | 25.057 | 0.13 | 14.576 |
| 10 | 2Hex2Pent | 606.22 | 25.746 | 0.17 | 14.977 |
| 11 | 2Hex2Pent | 606.22 | 26.647 | 0.48 | 15.501 |
| 12 | 3Hex | 504.18 | 3.849 | 0.24 | 2.239 |
| 13 | 3Hex | 504.18 | 5.343 | 0.33 | 3.108 |
| 14 | 3Hex | 504.19 | 11.767 | 5.38 | 6.845 |
| 15 | 3Hex | 504.19 | 15.441 | 6.96 | 8.983 |
| 16 | 3Hex | 504.18 | 18.026 | 4.45 | 10.486 |
| 17 | 3Hex | 504.18 | 25.418 | 0.87 | 14.787 |
| 18 | 3Hex | 504.18 | 26.295 | 0.69 | 15.297 |
| 19 | 3Hex1HexA | 680.22 | 24.739 | 0.81 | 14.392 |
| 20 | 3Hex1HexA | 680.22 | 26.958 | 1.14 | 15.682 |
| 21 | 3Hex1HexA | 680.22 | 27.89 | 0.77 | 16.225 |
| 22 | 3Hex1Pent | 636.23 | 23.178 | 1 | 13.483 |
| 23 | 3Hex1Pent | 636.23 | 23.633 | 0.2 | 13.748 |
| 24 | 3Hex1Pent | 636.23 | 24.263 | 0.91 | 14.115 |
| 25 | 3Hex1Pent | 636.23 | 25.062 | 0.92 | 14.579 |
| 26 | 3Hex1Pent | 636.23 | 25.67 | 1.39 | 14.933 |
| 27 | 3Hex1Pent | 636.23 | 25.953 | 0.18 | 15.098 |
| 28 | 3Hex2Pent | 768.27 | 30.747 | 0.47 | 17.887 |
| 29 | 4Hex | 666.24 | 8.262 | 0.13 | 4.806 |
| 30 | 4Hex | 666.24 | 9.807 | 0.41 | 5.705 |
| 31 | 4Hex | 666.24 | 10.743 | 0.23 | 6.25 |
| 32 | 4Hex | 666.24 | 10.95 | 0.23 | 6.37 |
| 33 | 4Hex | 666.24 | 21.681 | 8.58 | 12.613 |
| 34 | 4Hex | 666.24 | 25.165 | 5.48 | 14.639 |
| 35 | 4Hex | 666.24 | 25.416 | 6.17 | 14.785 |
| 36 | 4Hex | 666.24 | 26.296 | 5.74 | 15.297 |
| 37 | 4Hex1Pent | 798.28 | 28.67 | 0.36 | 16.678 |
| 38 | 4Hex1Pent | 798.28 | 28.936 | 0.67 | 16.833 |
| 39 | 4Hex1Pent | 798.28 | 29.484 | 1.28 | 17.152 |
| 40 | 4Hex1Pent | 798.28 | 30.066 | 1.94 | 17.49 |
| 41 | 5Hex | 828.29 | 27.779 | 3.22 | 16.16 |
| 42 | 5Hex | 828.29 | 28.284 | 3.66 | 16.454 |
| 43 | 5Hex | 828.29 | 29.192 | 1.64 | 16.982 |
| 44 | 5Hex | 828.29 | 29.692 | 6.88 | 17.273 |
| 45 | 5Hex | 828.29 | 30.357 | 10.05 | 17.66 |
| 46 | 5Hex1Pent | 960.33 | 33.396 | 0.87 | 19.428 |
| 47 | 6Hex | 990.34 | 15.87 | 0.27 | 9.232 |
| 48 | 6Hex | 990.36 | 26.295 | 0.15 | 15.297 |
| 49 | 6Hex | 990.34 | 33.413 | 6.11 | 19.437 |

TABLE N
Glycosidic linkage analysis of CLX compositions. Data are presented in units of peak area %. “Other”
refers to linkages making up less than 2%. The notation “—” represents a linkage that
exists in an amount less than 2% (which can be 0%) of the total oligosaccharide weight. If linkage
is not fully described it will be denoted by the monosaccharide, when known, or the type of monosaccharide,
either pentose or hexose, followed by multiple or a single “x” denoting the number of
branch points and finally the retention time, in parentheses, in the units of minutes.

| CLX No. | 3-Glc | 4-Glc | 6-Glc | 4,6-Glc | T-Glc | T-Man | 3-Gal | 4-Gal | 6-Gal | 3,6-Gal | T-Gal-p |
|---|---|---|---|---|---|---|---|---|---|---|---|
| CLX131 | — | — | — | — | 4.32 | — | — | 4.73 | — | — | 2.36 |
| CLX117 | — | 6.74 | — | — | 2.41 | — | — | 2.46 | — | 4.23 | — |
| CLX130 | 5.98 | 46.48 | — | — | 15.29 | — | — | — | — | — | 3.55 |
| CLX129 | 10.16 | — | 7.79 | — | 10.9 | 2.88 | — | — | — | — | 2.1 |
| CLX119 | — | 2.91 | — | — | 5.24 | — | — | 14.55 | — | — | 3.94 |
| CLX126 | — | 3.28 | — | — | — | — | — | 41.54 | — | — | 5.28 |
| CLX123 | 3.28 | 29.88 | — | — | 11.54 | 5.53 | — | — | — | 12.24 | — |
| CLX122 | — | 3.4 | — | — | — | — | — | 4.54 | — | — | 2.89 |
| CLX128 | 3.44 | 15.54 | — | — | 8.94 | — | — | — | — | — | — |
| CLX115A | 5.39 | 12 | 4.66 | — | 9.95 | 24.64 | — | — | — | — | — |
| CLX115AL | 3.95 | 25.15 | 9.09 | — | 6.84 | 6.74 | — | — | — | — | — |
| CLX125 | 20.96 | 24.24 | — | — | 10.25 | — | — | — | — | — | — |
| CLX124 | — | — | — | — | 2.26 | 8.86 | 9.37 | — | 3.78 | 6.98 | 9.13 |
| CLX127 | — | 2.26 | — | — | 5.25 | — | — | 17.73 | — | — | 4.56 |
| CLX116 | — | — | — | — | 6.2 | — | — | 4.88 | — | — | 2.47 |
| CLX107 | — | — | — | — | — | — | — | 13.1 | — | — | 29.64 |
| CLX118 | 35.52 | 21.18 | 14.72 | — | 18.75 | — | — | — | — | — | — |
| CLX132 | — | 3.27 | — | — | — | — | — | 26.45 | 6 | 3 | 11.19 |
| CLX133 | — | 9.03 | — | 4.18 | 2.02 | — | — | — | — | — | 2.85 |
| CLX122DS | — | 3.71 | — | — | — | — | — | — | — | — | — |
| CLX122DSF | — | — | — | — | — | — | — | 2.1 | — | — | — |
| CLX121 | — | — | — | — | — | — | — | 61 | — | — | 21.2 |

| | | 4,6- | | | | | | 2,3,4- | 3,4- | | 3,5- |
|---|---|---|---|---|---|---|---|---|---|---|---|
| CLX No. | 2-Man | Man | 3-Man | 4-Man | 2-Xyl | T-Xyl | 4-Xyl | Xyl | Xyl | 5-Arab | Arab |
| CLX131 | — | — | — | — | — | — | — | — | — | 20.97 | 13.51 |
| CLX117 | — | — | — | — | 3.19 | 3.91 | — | — | — | 6.8 | 2.44 |
| CLX130 | 5.19 | — | — | — | — | — | 7.39 | — | — | — | — |
| CLX129 | — | — | — | — | 4.65 | 14.16 | 5.87 | — | — | — | — |
| CLX119 | — | — | — | — | — | — | 2.65 | — | — | 29.15 | 2.91 |
| CLX126 | — | — | — | — | — | — | — | — | — | 11.11 | 4.81 |
| CLX123 | 16.02 | 6.09 | — | — | — | — | — | — | — | — | — |
| CLX122 | — | — | — | — | — | — | — | — | — | 23.16 | 4.2 |
| CLX128 | — | — | — | — | — | 5.97 | 27.17 | — | 4.81 | 7 | — |
| CLX115A | 23.39 | — | 2.44 | 2.44 | — | — | — | — | — | — | — |
| CLX115AL | 24.97 | — | 2.21 | 2.22 | — | — | — | — | — | 2.59 | — |
| CLX125 | — | — | — | — | — | — | — | — | — | — | — |
| CLX124 | — | — | — | 22.84 | — | — | — | — | — | 4.87 | — |
| CLX127 | — | — | — | — | — | — | — | — | — | 18.64 | 7.89 |
| CLX116 | — | — | — | — | — | — | — | — | — | 26.67 | 12.27 |
| CLX107 | — | — | — | — | — | — | — | — | — | — | — |
| CLX118 | — | — | — | — | — | — | — | — | — | — | — |
| CLX132 | — | — | — | — | — | — | — | — | — | 4.07 | — |
| CLX133 | — | — | — | — | 10.55 | 15.49 | 5.82 | 13.06 | — | 2.71 | — |
| CLX122DS | — | — | — | — | — | 1.48 | — | — | — | 21.21 | 5.13 |
| CLX122DSF | — | — | — | — | — | 2 | — | 10.04 | — | 17 | 3.84 |
| CLX121 | — | — | — | — | — | — | — | — | — | 5.3 | — |

| | | | | | | | | | | 2,x,x- | |
|---|---|---|---|---|---|---|---|---|---|---|---|
| | | | | | | x-Fuc | | | | Rham | |
| | 2,5- | 2,3,5- | T- | | | (9.503 | | | | (1.626 | |
| CLX No. | Arab | Arab | Arab-f | 2-Fuc | T-Fuc | min) | 2-Rham | 4-Rham | T-Rham | min) | 4-GlcA |
| CLX131 | — | — | 21.67 | — | — | — | — | — | — | — | — |
| CLX117 | 4.32 | 8.1 | 25.46 | — | 2.37 | — | — | — | — | — | — |
| CLX130 | — | — | — | — | — | — | — | — | — | — | — |
| CLX129 | — | — | — | 4.97 | 13.71 | 5.62 | — | — | — | — | — |
| CLX119 | — | — | 7.57 | — | — | — | 8.69 | — | — | — | — |
| CLX126 | — | 2.2 | 13.98 | — | — | — | — | — | — | — | — |
| CLX123 | — | — | — | — | — | — | — | — | — | — | 4.12 |
| CLX122 | 9.25 | 4.38 | 34.99 | — | — | — | — | — | — | — | — |
| CLX128 | — | — | 8.31 | 5.97 | — | — | — | — | — | — | — |
| CLX115A | — | — | — | — | — | — | — | — | — | — | — |
| CLX115AL | — | — | — | — | — | — | — | — | — | — | — |
| CLX125 | — | — | — | — | — | — | — | 21.57 | 7.33 | — | 4.91 |
| CLX124 | — | — | 15.54 | — | — | — | — | — | — | — | — |
| CLX127 | 3.56 | 3.54 | 27.39 | — | — | — | — | — | — | — | — |
| CLX116 | — | — | 20.64 | — | — | — | 3.66 | — | — | — | — |
| CLX107 | — | — | 4.02 | — | — | — | 16.16 | — | 11.31 | — | — |
| CLX118 | — | — | — | — | — | — | — | — | — | — | — |
| CLX132 | — | — | 7 | — | — | — | 9.53 | — | — | — | — |
| CLX133 | — | 4.67 | 4.25 | — | — | — | — | 2.21 | — | 2.17 | — |
| CLX122DS | 6.36 | 3.27 | 47.11 | — | — | — | — | — | — | — | — |
| CLX122DSF | 1.75 | — | 50.18 | — | — | — | — | — | — | — | — |
| CLX121 | — | — | 4.06 | — | — | — | — | — | — | — | — |

| | | | 2,x-Hex | x-Hex | 2-Pent | x,x-Hex | x-Pent | x-Pent | 2,x-Pent | |
|---|---|---|---|---|---|---|---|---|---|---|
| | | | (1.628 | (2.108 | (2.769 | (3.755 | (5.289 | (6.448 | (3.346 | |
| CLX No. | T-GlcA | T-GalA | min) | min) | min) | min) | min) | min) | min) | Other |
| CLX131 | — | — | — | — | — | — | 7.88 | — | — | 1.55 |
| CLX117 | — | — | — | 2.55 | 2.37 | — | 3.3 | 5.92 | — | 4.97 |
| CLX130 | — | — | — | — | — | — | — | — | — | 3.54 |
| CLX129 | — | — | — | — | — | 2.95 | — | — | — | 2.55 |
| CLX119 | — | — | — | 3.45 | — | — | — | — | — | 2.73 |
| CLX126 | — | — | — | — | — | — | — | — | — | 2.26 |
| CLX123 | — | — | — | — | — | — | — | — | — | 1.51 |
| CLX122 | — | — | — | — | — | — | — | — | — | 0.45 |
| CLX128 | 2 | — | — | — | — | — | — | — | — | 2.03 |
| CLX115A | — | — | 7.21 | — | — | 2.2 | — | — | — | 1.45 |
| CLX115AL | — | — | — | — | — | — | — | — | — | 11.27 |
| CLX125 | 2.5 | — | — | — | — | — | — | — | — | 1.16 |
| CLX124 | — | — | — | — | — | — | — | 2.04 | — | 2.37 |
| CLX127 | — | — | — | — | — | — | — | — | — | 2.6 |
| CLX116 | — | — | — | — | — | — | 2.89 | 2.34 | — | 1.76 |
| CLX107 | 11.18 | — | — | 5.94 | — | — | — | — | — | 3.26 |
| CLX118 | — | — | — | — | — | 5.68 | — | — | — | 0.9 |
| CLX132 | 2.85 | 3.31 | — | 6.07 | 3.84 | — | — | — | — | 1.76 |
| CLX133 | — | — | — | — | — | — | — | — | 2.64 | 5.39 |
| CLX122DS | — | — | — | — | — | — | — | — | — | 4.65 |
| CLX122DSF | — | — | — | — | — | — | — | — | — | 9.01 |
| CLX121 | — | — | — | — | — | — | — | — | — | 2.38 |

TABLE O
1H-13C HSQC NMR correlations for CLX compositions disclosed herein. The listed pairs
correspond to those major peaks in the anomeric region of the NMR spectrum.

| CLX107 | | CLX115A | | CLX115AL | | CLX116 | | CLX117 | | CLX118 | |
|---|---|---|---|---|---|---|---|---|---|---|---|
| ¹H δ | ¹³C δ | ¹H δ | ¹³C δ | ¹H δ | ¹³C δ | ¹H δ | ¹³C δ | ¹H δ | ¹³C δ | ¹H δ | ¹³C δ |
| ppm | ppm | ppm | ppm | ppm | ppm | ppm | ppm | ppm | ppm | ppm | ppm |
| 5.12 | 101.1 | 5.02 | 101.63 | 4.99 | 92.50 | 5 | 107.84 | 5.18 | 107.78 | 5.02 | 100.98 |
| 5.09 | 101.1 | 5.00 | 101.84 | 4.97 | 100.63 | 4.94 | 108.46 | 5.06 | 109.56 | 5.00 | 101.20 |
| 5.09 | 98.63 | 4.88 | 94.17 | 4.93 | 91.73 | 4.91 | 108.23 | 5.00 | 107.20 | 4.97 | 92.22 |
| 4.95 | 91.23 | 4.87 | 103.04 | 4.86 | 101.75 | 4.88 | 99.79 | 4.93 | 107.66 | 4.90 | 92.69 |
| 4.87 | 93.56 | 4.85 | 95.35 | 4.86 | 92.17 | 4.86 | 108.35 | 4.91 | 106.92 | 4.88 | 93.58 |
| 4.80 | 94.48 | 4.85 | 103.17 | 4.83 | 93.88 | 4.84 | 107.88 | 4.91 | 107.51 | 4.52 | 103.56 |
| 4.21 | 97.95 | 4.83 | 103.35 | 4.81 | 102.04 | 4.83 | 99.88 | 4.86 | 107.60 | 4.48 | 103.67 |
| 4.21 | 75.84 | 4.52 | 104.17 | 4.68 | 99.12 | 4.82 | 109.47 | 4.83 | 107.17 | 4.43 | 103.91 |
| 4.20 | 75.93 | 4.36 | 104.92 | 4.50 | 94.16 | 4.77 | 109.16 | 4.82 | 108.62 | 4.37 | 96.82 |
| 4.18 | 106.63 | 4.24 | 104.33 | 4.47 | 103.06 | 4.55 | 101.69 | 4.77 | 108.40 | 4.36 | 96.82 |
| 4.17 | 76.11 | 3.98 | 69.44 | 4.39 | 103.40 | 4.53 | 101.58 | 4.19 | 103.96 | 4.36 | 104.37 |
| 4.00 | 70.95 | 3.57 | 69.49 | 4.33 | 96.32 | 4.26 | 97.96 | 4.17 | 103.93 | 4.34 | 103.06 |
| 3.96 | 70.9 | 3.71 | 67.11 | 4.32 | 103.82 | 4.26 | 106.53 | 3.66 | 67.03 | 4.33 | 104.58 |
| | | 3.66 | 67.08 | 4.25 | 103.93 | | | 3.64 | 67.40 | 4.30 | 104.54 |
| | | 3.53 | 66.55 | 4.22 | 96.86 | | | 3.55 | 67.04 | 4.29 | 104.56 |
| | | | | | | | | 3.52 | 67.38 | 4.27 | 97.38 |
| | | | | | | | | | | 4.26 | 97.38 |
| | | | | | | | | | | 4.24 | 103.78 |
| | | | | | | | | | | 4.18 | 103.65 |
| | | | | | | | | | | 4.17 | 103.59 |
| | | | | | | | | | | 4.15 | 104.04 |
| | | | | | | | | | | 3.98 | 68.80 |
| | | | | | | | | | | 3.57 | 68.80 |
| | | | | | | | | | | 3.46 | 86.75 |
| | | | | | | | | | | 3.42 | 87.28 |
| | | | | | | | | | | 3.35 | 88.43 |
| | | | | | | | | | | 3.44 | 79.92 |

| CLX119 | | CLX122 | | CLX123 | | CLX124 | | CLX125 | |
|---|---|---|---|---|---|---|---|---|---|
| ¹H δ | ¹³C δ | ¹H δ | ¹³C δ | ¹H δ | ¹³C δ | ¹H δ | ¹³C δ | ¹H δ | ¹³C δ |
| ppm | ppm | ppm | ppm | ppm | ppm | ppm | ppm | ppm | ppm |
| 5.18 | 92.81 | 5.00 | 107.17 | 5.01 | 93.22 | 5.06 | 109.5 | 5.12 | 99.92 |
| 5.09 | 101.64 | 4.93 | 107.83 | 5.00 | 101.92 | 4.90 | 94.30 | 5.08 | 100.49 |
| 5.06 | 99.35 | 4.90 | 105.88 | 4.97 | 102.02 | 4.77 | 108.67 | 5.05 | 100.46 |
| 5.04 | 93.46 | 4.90 | 106.90 | 4.97 | 102.29 | 4.76 | 108.4 | 5.01 | 100.49 |
| 4.90 | 108.24 | 4.86 | 107.67 | 4.91 | 93.19 | 4.53 | 101.16 | 5.00 | 101.01 |
| 4.89 | 99.78 | 4.83 | 107.22 | 4.88 | 94.14 | 4.49 | 104.7 | 4.97 | 100.95 |
| 4.85 | 99.78 | 4.81 | 108.63 | 4.87 | 95.07 | 4.47 | 101.16 | 4.90 | 92.50 |
| 4.82 | 99.74 | 4.77 | 107.44 | 4.74 | 101.59 | 4.40 | 105.35 | 4.88 | 92.79 |
| 4.82 | 102.31 | 4.77 | 108.55 | 4.73 | 99.99 | 4.20 | 103.98 | 4.80 | 94.54 |
| 4.82 | 109.49 | 4.26 | 105.83 | 4.71 | 101.77 | 4.15 | 104.22 | 4.43 | 104.69 |
| 4.77 | 109.21 | 4.04 | 85.79 | 4.66 | 99.74 | 3.88 | 103.50 | 4.36 | 105.04 |
| 4.74 | 109.17 | 3.92 | 87.99 | 4.64 | 99.82 | 3.74 | 112.41 | 4.30 | 102.1 |
| 4.73 | 98.21 | 4.63 | 101.54 | 3.42 | 99.83 | 4.29 | 97.07 | 3.93 | 88.01 |
| 4.67 | 100.78 | 4.38 | 103.77 | 3.22 | 97.84 | 4.29 | 104.52 | 3.75 | 79.18 |
| 4.63 | 100.85 | 4.34 | 103.32 | | | 4.24 | 102.68 | 3.70 | 67.26 |
| 4.29 | 98.28 | 4.31 | 97.82 | | | | | 3.65 | 66.74 |
| 4.26 | 98.14 | 4.31 | 103.77 | | | | | 3.63 | 67.51 |
| 4.27 | 102.81 | 4.27 | 98.04 | | | | | 3.54 | 67.16 |
| 4.26 | 106.53 | 4.25 | 103.47 | | | | | 3.54 | 66.61 |
| | | 3.84 | 64.91 | | | | | 3.52 | 67.48 |
| | | 3.66 | 64.86 | | | | | | |
| CLX126 | | CLX127 | | CLX128 | | CLX129 | | CLX130 | |
| ¹H δ | ¹³C δ | ¹H δ | ¹³C δ | ¹H δ | ¹³C δ | ¹H δ | ¹³C δ | ¹H δ | ¹³C δ |
| ppm | ppm | ppm | ppm | ppm | ppm | ppm | ppm | ppm | ppm |
| 4.99 | 107.2 | 5.18 | 92.20 | 5.34 | 107.75 | 4.86 | 93.71 | 5.18 | 92.15 |
| 4.93 | 107.81 | 4.99 | 107.17 | 5.20 | 108.06 | 4.84 | 93.65 | 4.88 | 93.55 |
| 4.90 | 106.86 | 4.94 | 107.80 | 5.18 | 92.33 | 4.87 | 95.13 | 4.76 | 94.51 |
| 4.91 | 107.59 | 4.90 | 107.63 | 5.08 | 97.86 | 4.88 | 99.91 | 4.73 | 99.95 |
| 4.86 | 107.64 | 4.90 | 106.93 | 4.90 | 92.64 | 4.90 | 102.73 | 4.68 | 99.97 |
| 4.83 | 107.22 | 4.86 | 107.68 | 4.42 | 104.18 | 4.62 | 102.06 | 4.35 | 104.68 |
| 4.82 | 108.8 | 4.83 | 107.27 | 4.37 | 103.14 | 4.61 | 100.85 | 4.33 | 104.43 |
| 4.79 | 108.68 | 4.82 | 108.75 | 4.36 | 103.22 | 4.59 | 100.02 | 4.32 | 104.43 |
| 4.78 | 108.53 | 4.79 | 108.65 | 4.32 | 104.29 | 4.57 | 99.89 | 4.33 | 103.87 |
| 4.26 | 105.84 | 4.76 | 108.54 | 4.32 | 103.30 | 4.57 | 105.48 | 4.22 | 103.92 |
| 4.23 | 105.98 | 4.29 | 105.98 | 4.31 | 102.08 | 4.54 | 101.95 | 4.21 | 103.90 |
| 4.04 | 85.82 | 4.26 | 105.93 | 4.31 | 104.34 | 4.53 | 101.99 | | |
| 4.23 | 106.25 | 4.30 | 103.27 | 4.52 | 104.19 | | | | |
| 4.04 | 85.87 | 4.28 | 103.22 | 4.40 | 103.84 | | | | |
| 3.93 | 88.07 | 4.26 | 101.32 | 4.38 | 104.56 | | | | |
| 3.75 | 79.21 | 4.26 | 102.21 | 4.27 | 104.25 | | | | |
| 3.71 | 67.35 | 4.24 | 98.06 | 4.23 | 104.33 | | | | |
| 3.67 | 66.73 | 4.24 | 103.71 | 4.22 | 104.41 | | | | |
| 3.65 | 66.83 | 4.24 | 102.28 | 4.21 | 104.44 | | | | |
| 3.62 | 67.35 | 4.23 | 103.81 | 4.19 | 98.44 | | | | |
| 3.57 | 67.20 | 4.22 | 98.02 | 4.16 | 104.63 | | | | |
| 3.55 | 67.17 | 4.22 | 102.36 | 4.15 | 104.15 | | | | |
| 3.53 | 67.14 | 4.20 | 102.24 | 4.07 | 104.75 | | | | |
| 3.51 | 67.45 | 4.17 | 102.24 | | | | | | |
| | | 3.97 | 86.68 | | | | | | |
| | | 3.70 | 66.28 | | | | | | |
| | | 3.06 | 66.35 | | | | | | |

| CLX131 | | CLX132 | | CLX122DS | | CLX122DSF | | CLX121 | | CLX133 | |
|---|---|---|---|---|---|---|---|---|---|---|---|
| ¹H δ | ¹³C δ | ¹H δ | ¹³C δ | ¹H δ | ¹³C δ | ¹H δ | ¹³C δ | ¹H δ | ¹³C δ | ¹H δ | ¹³C δ |
| ppm | ppm | ppm | ppm | ppm | ppm | ppm | ppm | ppm | ppm | ppm | ppm |
| 4.96 | 107.92 | 5.18 | 92.19 | 5.01 | 101.79 | 5.17 | 100.60 | 4.93 | 107.78 | 5.17 | 92.16 |
| 4.91 | 108.24 | 5.00 | 107.24 | 5.00 | 107.83 | 5.08 | 95.20 | 4.91 | 93.08 | 5.06 | 98.61 |
| 4.83 | 102.59 | 4.91 | 107.59 | 4.94 | 108.47 | 5.07 | 100.12 | 4.90 | 106.74 | 4.99 | 101.05 |
| 4.82 | 102.53 | 4.86 | 107.69 | 4.91 | 103.01 | 5.02 | 108.48 | 4.90 | 102.05 | 4.94 | 101.10 |
| 4.82 | 109.43 | 4.83 | 107.22 | 4.91 | 108.31 | 5.02 | 100.21 | 4.80 | 94.49 | 4.92 | 107.84 |
| 4.8 | 109.32 | 4.82 | 108.76 | 4.90 | 107.50 | 5.01 | 92.27 | 4.76 | 108.46 | 4.91 | 100.94 |
| 4.78 | 109.27 | 4.77 | 108.55 | 4.86 | 108.32 | 4.99 | 100.39 | 4.75 | 94.68 | 4.90 | 101.88 |
| 4.76 | 109.21 | 3.70 | 67.44 | 4.83 | 107.83 | 4.99 | 95.51 | 4.73 | 94.83 | 4.90 | 92.58 |
| 7.75 | 109.11 | 3.64 | 67.32 | 4.82 | 109.38 | 4.99 | 92.13 | 4.34 | 105.93 | 4.88 | 99.10 |
| 4.57 | 99.93 | 3.55 | 67.23 | 4.78 | 109.21 | 4.97 | 106.14 | 4.26 | 103.77 | 4.88 | 101.30 |
| | | 3.53 | 67.50 | 4.26 | 98.10 | 4.96 | 101.79 | 4.26 | 105.87 | 4.85 | 92.92 |
| | | | | | | 4.92 | 107.05 | 4.24 | 106.21 | 4.82 | 98.82 |
| | | | | | | 4.90 | 101.62 | 3.71 | 67.27 | 4.76 | 108.39 |
| | | | | | | 4.90 | 98.46 | 3.65 | 67.34 | 4.73 | 97.45 |
| | | | | | | 4.90 | 92.05 | 3.64 | 67.53 | 4.63 | 99.53 |
| | | | | | | 4.89 | 106.52 | 3.55 | 67.17 | 4.55 | 100.76 |
| | | | | | | 4.87 | 101.19 | 3.52 | 67.53 | 4.48 | 101.25 |
| | | | | | | 4.85 | 107.00 | 3.51 | 67.50 | 4.34 | 96.73 |
| | | | | | | 4.85 | 92.48 | | | 4.33 | 103.23 |
| | | | | | | 4.82 | 106.57 | | | 4.32 | 96.94 |
| | | | | | | 4.80 | 93.82 | | | 4.26 | 102.21 |
| | | | | | | 4.76 | 107.75 | | | 4.26 | 97.36 |
| | | | | | | 4.72 | 94.18 | | | 4.25 | 106.07 |
| | | | | | | 4.63 | 98.84 | | | 4.25 | 103.82 |
| | | | | | | 4.32 | 102.54 | | | 4.21 | 97.94 |
| | | | | | | 4.31 | 96.51 | | | 4.19 | 103.87 |
| | | | | | | 4.26 | 102.05 | | | | |
| | | | | | | 4.26 | 96.68 | | | | |
| | | | | | | 4.24 | 105.55 | | | | |
| | | | | | | 4.23 | 102.75 | | | | |
| | | | | | | 4.21 | 97.16 | | | | |

In each of the tables below, and elsewhere herein where these terms are used, Hex refers to hexose sugars, Pent refers to pentose sugars, HexA refers to hexuronic acid sugars, and Deoxyhex refers to deoxyhexose sugars. The number preceding such designation refers to the number of those units present in the relevant oligosaccharide (e.g., “3Hex” means the oligosaccharide is an oligomer of three hexoses). NR refers to an oligosaccharide without a reducing end. RT refers to retention time. Oligo wt. % is short for oligosaccharide weight % as that term is used in the definition of “oligosaccharide analysis” elsewhere herein.

TABLE P
CLX 115A comprises the oligosaccharides shown in this
table (Hex = a hexose, generally glucose or mannose).

| Com- | | | RT | | Retention |
|---|---|---|---|---|---|
| pound | Identity | Mass | (min) | Oligo Wt. % | Factor |
| 1 | 3Hex | 504.18 | 1.225 | 3.04 | 1 |
| 2 | 3Hex | 504.19 | 4.863 | 13.2 | 3.97 |
| 3 | 3Hex | 504.19 | 10.984 | 5.24 | 8.967 |
| 4 | 3Hex | 504.19 | 11.677 | 1.3 | 9.532 |
| 5 | 3Hex | 504.19 | 13.16 | 16.41 | 10.743 |
| 6 | 4Hex | 666.24 | 1.497 | 7.99 | 1.222 |
| 7 | 4Hex | 666.24 | 10.916 | 13.88 | 8.911 |
| 8 | 4Hex | 666.24 | 16.918 | 2.32 | 13.811 |
| 9 | 4Hex | 666.24 | 24.788 | 9.9 | 20.235 |
| 10 | 5Hex | 828.29 | 1.926 | 10.61 | 1.572 |
| 11 | 5Hex | 828.29 | 14.015 | 8.82 | 11.441 |
| 12 | 5Hex | 828.29 | 30.092 | 7.29 | 24.565 |

TABLE P2
CLX 115AL comprises the oligosaccharides shown in this
table (Hex = a hexose, generally glucose or mannose).

| Com- | | | RT | | Retention |
|---|---|---|---|---|---|
| pound | Identity | Mass | (min) | Oligo Wt. % | Factor |
| 1 | 3Hex | 504.18 | 13.747 | 33.84 | 1.25 |
| 2 | 4Hex | 666.24 | 10.996 | 18.04 | 1 |
| 3 | 4Hex | 666.24 | 25.264 | 28.89 | 2.298 |
| 4 | 5Hex | 828.29 | 13.896 | 19.23 | 1.264 |

TABLE Q
CLX 117 comprises the oligosaccharides shown in this table.

| Com- | | | RT | | Retention |
|---|---|---|---|---|---|
| pound | Identity | Mass | (min) | Oligo Wt. % | Factor |
| 1 | 1Hex2Pent | 444.16 | 4.516 | 2.42 | 1.432 |
| 2 | 1Hex2Pent | 444.16 | 4.734 | 1.56 | 1.501 |
| 3 | 1Hex2Pent | 444.16 | 4.739 | 2.63 | 1.503 |
| 4 | 1Hex3Pent | 576.2 | 11.892 | 6.24 | 3.77 |
| 5 | 1Hex3Pent | 576.25 | 14.374 | 2.45 | 4.557 |
| 6 | 2Hex1Pent | 474.17 | 3.674 | 1.36 | 1.165 |
| 7 | 2Hex1Pent | 474.17 | 4.732 | 1.99 | 1.5 |
| 8 | 2Hex1Pent | 474.17 | 14.418 | 1.73 | 4.571 |
| 9 | 2Hex2Pent | 606.22 | 9.863 | 0.96 | 3.127 |
| 10 | 2Hex2Pent | 606.21 | 14.391 | 1.19 | 4.563 |
| 11 | 3Hex | 504.18 | 3.154 | 1.11 | 1 |
| 12 | 3Hex | 504.18 | 4.762 | 7.88 | 1.51 |
| 13 | 3Hex (NR) | 504.22 | 14.19 | 0.98 | 4.499 |
| 14 | 3Hex1Pent | 636.22 | 8.485 | 0.97 | 2.69 |
| 15 | 3Hex1Pent | 636.23 | 10.474 | 2.34 | 3.321 |
| 16 | 3Pent | 414.15 | 3.893 | 4.61 | 1.234 |
| 17 | 3Pent | 414.15 | 5.253 | 1.61 | 1.666 |
| 18 | 3Pent | 414.15 | 6.251 | 7.19 | 1.982 |
| 19 | 3Pent | 414.15 | 8.361 | 2.53 | 2.651 |
| 20 | 3Pent | 414.15 | 10.568 | 3.66 | 3.351 |
| 21 | 4Hex | 666.24 | 7.796 | 1.29 | 2.472 |
| 22 | 4Hex | 666.24 | 10.425 | 10.58 | 3.305 |
| 23 | 4Hex1Pent | 798.28 | 11.282 | 0.99 | 3.577 |
| 24 | 4Hex1Pent | 798.28 | 13.425 | 1.83 | 4.256 |
| 25 | 4Pent | 546.19 | 11.299 | 7.59 | 3.582 |
| 26 | 4Pent | 546.19 | 15.593 | 1.2 | 4.944 |
| 27 | 4Pent | 546.19 | 17.068 | 2.27 | 5.412 |
| 28 | 5Hex | 828.29 | 10.82 | 0.71 | 3.431 |
| 29 | 5Hex | 828.29 | 13.328 | 7.84 | 4.226 |
| 30 | 5Hex1Pent | 960.33 | 15.093 | 1.21 | 4.785 |
| 31 | 6Hex | 990.34 | 15.002 | 5.27 | 4.756 |
| 32 | 6Pent | 810.28 | 18.661 | 1.47 | 5.917 |
| 33 | 7Hex | 1152.39 | 16.135 | 2.35 | 5.116 |

TABLE R
CLX 118 comprises the oligosaccharides shown in this table.

| Com- | | | RT | | Retention |
|---|---|---|---|---|---|
| pound | Identity | Mass | (min) | Oligo Wt. % | Factor |
| 1 | 3Hex | 504.19 | 4.752 | 7.87 | 1 |
| 2 | 3Hex | 504.19 | 8.159 | 1.79 | 1.717 |
| 3 | 3Hex | 504.19 | 10.738 | 9.68 | 2.26 |
| 4 | 3Hex | 504.19 | 11.389 | 2.16 | 2.397 |
| 5 | 3Hex | 504.19 | 12.883 | 17.48 | 2.711 |
| 6 | 3Hex | 504.19 | 15.154 | 0.63 | 3.189 |
| 7 | 3Hex | 504.19 | 16.363 | 1.92 | 3.443 |
| 8 | 4Hex | 666.24 | 10.629 | 7.59 | 2.237 |
| 9 | 4Hex | 666.24 | 14.611 | 1.01 | 3.075 |
| 10 | 4Hex | 666.24 | 16.655 | 5.87 | 3.505 |
| 11 | 4Hex | 666.24 | 21.124 | 0.85 | 4.445 |
| 12 | 4Hex | 666.24 | 21.668 | 1.62 | 4.56 |
| 13 | 4Hex | 666.24 | 24.508 | 11.40 | 5.157 |
| 14 | 4Hex | 666.24 | 24.804 | 1.71 | 5.22 |
| 15 | 5Hex | 828.29 | 13.764 | 7.65 | 2.896 |
| 16 | 5Hex | 828.29 | 20.094 | 3.59 | 4.229 |
| 17 | 5Hex | 828.29 | 29.904 | 2.84 | 6.293 |
| 18 | 6Hex | 990.34 | 15.62 | 4.97 | 3.287 |
| 19 | 6Hex | 990.34 | 23.157 | 1.83 | 4.873 |
| 20 | 6Hex | 990.34 | 34.633 | 5.89 | 7.288 |
| 21 | 7Hex | 1152.4 | 16.846 | 1.64 | 3.545 |

TABLE S
CLX 119 comprises the oligosaccharides shown in this table.

| | | | | | Reten- |
|---|---|---|---|---|---|
| Com- | | | RT | Oligo | tion |
| pound | Identity | Mass | (min) | Wt. % | Factor |
| 1 | 3Hex | 504.19 | 3.081 | 6.11 | 1 |
| 2 | 3Hex | 504.19 | 10.081 | 4.70 | 3.272 |
| 3 | 3Hex (NR) | 504.17 | 4.531 | 75.48 | 1.471 |
| 4 | 4Hex | 666.24 | 8.191 | 5.62 | 2.659 |
| 5 | 4Hex | 666.24 | 19.727 | 3.68 | 6.403 |
| 6 | 5Hex | 828.29 | 11.549 | 4.41 | 3.748 |

TABLE T
CLX 121 comprises the oligosaccharides shown in this table
(Hex = hexose, generally galactose, pent = pentose generally
Arabinose and Hex A = Hexuronic acid generally galacturnic acid).

| | | | | | Reten- |
|---|---|---|---|---|---|
| Com- | | | RT | Oligo | tion |
| pound | Identity | Mass | (min) | Wt. % | Factor |
| 1 | 2Hex1HexA | 518.17 | 4.698 | 3.82 | 1.536 |
| 2 | 2Hex1Pent | 474.18 | 3.058 | 0.80 | 1 |
| 3 | 2Hex1Pent | 474.18 | 3.57 | 9.46 | 1.167 |
| 4 | 2Hex1Pent | 474.17 | 3.886 | 0.86 | 1.271 |
| 5 | 3Hex | 504.19 | 3.098 | 10.67 | 1.013 |
| 6 | 3Hex | 504.19 | 4.06 | 1.10 | 1.328 |
| 7 | 3Hex1HexA | 680.22 | 9.814 | 5.15 | 3.209 |
| 8 | 3Hex1Pent | 636.23 | 7.045 | 1.01 | 2.304 |
| 9 | 3Hex1Pent | 636.23 | 7.508 | 0.82 | 2.455 |
| 10 | 3Hex1Pent | 636.23 | 7.831 | 0.60 | 2.561 |
| 11 | 3Hex1Pent | 636.23 | 8.574 | 12.31 | 2.804 |
| 12 | 4Hex | 666.24 | 7.754 | 13.60 | 2.536 |
| 13 | 4Hex | 666.24 | 8.957 | 0.93 | 2.929 |
| 14 | 4Hex1HexA | 842.27 | 13.063 | 3.37 | 4.272 |
| 15 | 4Hex1Pent | 798.28 | 10.559 | 0.47 | 3.453 |
| 16 | 4Hex1Pent | 798.28 | 10.862 | 0.83 | 3.552 |
| 17 | 4Hex1Pent | 798.28 | 11.786 | 9.98 | 3.854 |
| 18 | 5Hex | 828.29 | 11.246 | 9.46 | 3.678 |
| 19 | 5Hex | 828.29 | 11.945 | 0.54 | 3.906 |
| 20 | 5Hex1HexA | 1004.32 | 14.971 | 2.22 | 4.896 |
| 21 | 5Hex1Pent | 960.33 | 13.016 | 0.53 | 4.256 |
| 22 | 5Hex1Pent | 960.33 | 13.697 | 5.16 | 4.479 |
| 23 | 6Hex | 990.34 | 13.315 | 2.17 | 4.354 |
| 24 | 6Hex1HexA | 1166.38 | 16.133 | 0.91 | 5.276 |
| 25 | 6Hex1Pent | 1122.38 | 14.908 | 1.83 | 4.875 |
| 26 | 7Hex | 1152.4 | 14.632 | 0.86 | 4.785 |
| 27 | 7Hex1Pent | 1284.44 | 16.044 | 0.53 | 5.247 |

TABLE U
CLX 122 comprises the oligosaccharides shown in this
table (Hex = hexose, generally galactose or mannose).

| | | | | | Reten- |
|---|---|---|---|---|---|
| Com- | | | RT | Oligo | tion |
| pound | Identity | Mass | (min) | Wt. % | Factor |
| 1 | 2Hex1Pent | 474.17 | 3.789 | 3.20 | 1.157 |
| 2 | 2Hex1Pent | 474.17 | 5.019 | 1.67 | 1.532 |
| 3 | 3Hex | 504.18 | 3.276 | 3.64 | 1 |
| 4 | 3Hex | 504.18 | 5.023 | 5.46 | 1.533 |
| 5 | 3Hex | 504.18 | 15.334 | 2.97 | 4.681 |
| 6 | 3Hex (NR) | 504.17 | 3.911 | 1.52 | 1.194 |
| 7 | 3Hex (NR) | 504.17 | 5.613 | 3.36 | 1.713 |
| 8 | 3Hex (NR) | 504.17 | 10.914 | 3.05 | 3.332 |
| 9 | 3Hex (NR) | 504.17 | 12.221 | 8.03 | 3.73 |
| 10 | 3Hex1Pent | 636.23 | 8.958 | 3.67 | 2.734 |
| 11 | 3Hex1Pent | 636.23 | 10.904 | 1.65 | 3.328 |
| 12 | 3Pent | 414.15 | 3.867 | 0.98 | 1.18 |
| 13 | 3Pent | 414.15 | 6.269 | 9.92 | 1.914 |
| 14 | 3Pent | 414.16 | 7.057 | 0.81 | 2.154 |
| 15 | 4Hex | 666.24 | 8.187 | 3.64 | 2.499 |
| 16 | 4Hex | 666.24 | 10.829 | 5.60 | 3.306 |
| 17 | 4Hex | 666.24 | 19.604 | 0.58 | 5.984 |
| 18 | 4Hex | 666.24 | 25.305 | 2.23 | 7.724 |
| 19 | 4Hex (NR) | 666.22 | 9.171 | 3.30 | 2.799 |
| 20 | 4Hex (NR) | 666.22 | 12.226 | 0.95 | 3.732 |
| 21 | 4Hex (NR) | 666.22 | 12.227 | 4.67 | 3.732 |
| 22 | 4Hex (NR) | 666.22 | 15.283 | 3.31 | 4.665 |
| 23 | 4Hex1Pent | 798.28 | 11.988 | 3.05 | 3.659 |
| 24 | 4Hex1Pent | 798.28 | 14.07 | 1.23 | 4.295 |
| 25 | 4Pent | 546.19 | 10.644 | 1.79 | 3.249 |
| 26 | 4Pent | 546.2 | 11.358 | 3.33 | 3.467 |
| 27 | 4Pent | 546.2 | 13.022 | 0.89 | 3.975 |
| 28 | 5Hex | 828.29 | 11.467 | 2.50 | 3.5 |
| 29 | 5Hex | 828.29 | 13.946 | 4.75 | 4.257 |
| 30 | 5Hex | 828.29 | 29.244 | 1.39 | 8.927 |
| 31 | 5Pent | 678.24 | 15.834 | 2.26 | 4.833 |
| 32 | 5Pent | 678.23 | 17.759 | 1.00 | 5.421 |
| 33 | 6Hex | 990.34 | 15.785 | 2.77 | 4.818 |
| 34 | 6Hex1Pent | 1122.39 | 15.078 | 0.83 | 4.603 |

TABLE U2
CLX 122DS comprises the oligosaccharides shown in this
table (Hex = hexose, generally galactose or glucose).

| | | | | | Reten- |
|---|---|---|---|---|---|
| Com- | | | RT | Oligo | tion |
| pound | Identity | Mass | (min) | Wt. % | Factor |
| 1 | 2Hex1Pent | 474.17 | 4.036 | 0.69 | 1.16 |
| 2 | 2Hex1Pent | 474.17 | 5.323 | 1.46 | 1.53 |
| 3 | 2Hex1Pent | 474.18 | 11.305 | 0.61 | 3.25 |
| 4 | 3Hex | 504.18 | 3.483 | 0.88 | 1 |
| 5 | 3Hex | 504.18 | 5.362 | 5.51 | 1.54 |
| 6 | 3Hex | 504.18 | 15.448 | 1.93 | 4.44 |
| 7 | 3Hex | 504.19 | 15.767 | 0.59 | 4.53 |
| 8 | 3Hex1Pent | 636.23 | 9.205 | 0.5 | 2.64 |
| 9 | 3Hex1Pent | 636.22 | 10.992 | 1.77 | 3.16 |
| 10 | 3Pent | 414.15 | 4.026 | 2.44 | 1.16 |
| 11 | 3Pent | 414.15 | 5.603 | 1.13 | 1.61 |
| 12 | 3Pent | 414.15 | 6.573 | 8.31 | 1.89 |
| 13 | 3Pent | 414.15 | 7.332 | 1.48 | 2.11 |
| 14 | 3Pent | 414.15 | 10.03 | 1.11 | 2.88 |
| 15 | 3Pent | 414.15 | 10.802 | 2.03 | 3.1 |
| 16 | 3Pent | 414.15 | 11.502 | 4.27 | 3.3 |
| 17 | 3Pent | 414.15 | 17.973 | 1.17 | 5.16 |
| 18 | 4Hex | 666.24 | 8.529 | 0.68 | 2.45 |
| 19 | 4Hex | 666.24 | 10.938 | 6.48 | 3.14 |
| 20 | 4Hex | 666.24 | 19.58 | 0.53 | 5.62 |
| 21 | 4Hex | 666.24 | 25.163 | 1.64 | 7.22 |
| 22 | 4Hex1Pent | 798.28 | 12.023 | 0.56 | 3.45 |
| 23 | 4Hex1Pent | 798.28 | 13.963 | 1.87 | 4.01 |
| 24 | 4Pent | 546.19 | 6.57 | 0.63 | 1.89 |
| 25 | 4Pent | 546.2 | 10.783 | 1.23 | 3.1 |
| 26 | 4Pent | 546.2 | 10.825 | 0.99 | 3.11 |
| 27 | 4Pent | 546.19 | 11.155 | 0.92 | 3.2 |
| 28 | 4Pent | 546.19 | 11.497 | 4.39 | 3.3 |
| 29 | 4Pent | 546.19 | 13.818 | 1.5 | 3.97 |
| 30 | 4Pent | 546.19 | 16.241 | 0.89 | 4.66 |
| 31 | 4Pent | 546.19 | 17.957 | 1.58 | 5.16 |
| 32 | 5Hex | 828.29 | 11.567 | 0.5 | 3.32 |
| 33 | 5Hex | 828.29 | 13.869 | 7.43 | 3.98 |
| 34 | 5Hex | 828.29 | 29.199 | 1.48 | 8.38 |
| 35 | 5Hex1Pent | 960.33 | 15.681 | 1.24 | 4.5 |
| 36 | 5Pent | 678.24 | 15.754 | 3.37 | 4.52 |
| 37 | 5Pent | 678.24 | 16.235 | 1.19 | 4.66 |
| 38 | 5Pent | 678.24 | 17.874 | 1.11 | 5.13 |
| 39 | 5Pent | 678.24 | 19.37 | 0.42 | 5.56 |
| 40 | 5Pent | 678.24 | 20.327 | 0.89 | 5.84 |
| 41 | 6Hex | 990.34 | 15.605 | 5.63 | 4.48 |
| 42 | 6Hex1Pent | 1122.38 | 16.842 | 0.74 | 4.84 |
| 43 | 6Pent | 810.32 | 6.567 | 0.47 | 1.89 |
| 44 | 6Pent | 810.28 | 17.192 | 0.6 | 4.94 |
| 45 | 6Pent | 810.28 | 18.685 | 1 | 5.36 |
| 46 | 6Pent | 810.28 | 19.018 | 1.77 | 5.46 |
| 47 | 6Pent | 810.28 | 19.725 | 1.79 | 5.66 |
| 48 | 7Hex | 1152.39 | 16.777 | 3.51 | 4.82 |
| 49 | 7Pent | 942.32 | 20.319 | 1.36 | 5.83 |
| 50 | 7Pent | 942.32 | 22.471 | 1.29 | 6.45 |
| 51 | 8Hex | 1314.45 | 17.774 | 2 | 5.1 |
| 52 | 8Pent | 1074.36 | 23.36 | 1.53 | 6.71 |
| 53 | 9Hex | 1476.5 | 19.142 | 0.91 | 5.5 |

TABLE U3
CLX 122DSF comprises the oligosaccharides shown in
this table (Hex = hexose, generally galactose
or glucose and Pent = Pentose generally Arabinose).

| | | | | | Reten- |
|---|---|---|---|---|---|
| Com- | | | RT | Oligo | tion |
| pound | Identity | Mass | (min) | Wt. % | Factor |
| 1 | 2Hex1Pent | 474.18 | 3.738 | 1.46 | 1.133 |
| 2 | 2Hex1Pent | 474.18 | 4.894 | 0.53 | 1.483 |
| 3 | 2Hex1Pent | 474.17 | 7.865 | 0.43 | 2.383 |
| 4 | 2Hex1Pent | 474.17 | 8.657 | 0.82 | 2.623 |
| 5 | 2Hex1Pent | 474.18 | 11.39 | 1 | 3.452 |
| 6 | 2Hex1Pent | 474.17 | 14.152 | 0.38 | 4.288 |
| 7 | 3Hex | 504.18 | 3.3 | 2.62 | 1 |
| 8 | 3Hex | 504.19 | 3.711 | 0.48 | 1.125 |
| 9 | 3Hex | 504.19 | 4.89 | 1.58 | 1.482 |
| 10 | 3Hex | 504.19 | 7.959 | 0.83 | 2.412 |
| 11 | 3Hex | 504.18 | 15.53 | 2.25 | 4.706 |
| 12 | 3Hex | 504.19 | 20.938 | 0.64 | 6.345 |
| 13 | 3Hex | 504.19 | 22.266 | 1.23 | 6.747 |
| 14 | 3Hex | 504.19 | 25.228 | 0.62 | 7.645 |
| 15 | 3Hex | 504.19 | 26.138 | 0.82 | 7.921 |
| 16 | 3Pent | 414.16 | 4.045 | 5.2 | 1.226 |
| 17 | 3Pent | 414.15 | 5.625 | 3.51 | 1.705 |
| 18 | 3Pent | 414.15 | 6.668 | 10.74 | 2.021 |
| 19 | 3Pent | 414.15 | 7.368 | 2.49 | 2.233 |
| 20 | 3Pent | 414.15 | 10.065 | 0.49 | 3.05 |
| 21 | 3Pent | 414.15 | 10.977 | 4.44 | 3.326 |
| 22 | 3Pent | 414.15 | 11.713 | 4.51 | 3.549 |
| 23 | 3Pent | 414.15 | 12.482 | 2.1 | 3.782 |
| 24 | 3Pent | 414.15 | 12.785 | 1.97 | 3.874 |
| 25 | 3Pent | 414.15 | 13.3 | 2.24 | 4.03 |
| 26 | 3Pent | 414.15 | 14.031 | 1.95 | 4.252 |
| 27 | 3Pent | 414.15 | 16.631 | 1.15 | 5.04 |
| 28 | 4Hex | 666.24 | 7.958 | 1.86 | 2.412 |
| 29 | 4Hex | 666.24 | 10.667 | 1.69 | 3.232 |
| 30 | 4Hex | 666.24 | 25.519 | 1.97 | 7.733 |
| 31 | 4Hex1Pent | 798.28 | 11.491 | 0.58 | 3.482 |
| 32 | 4Pent | 546.2 | 4.044 | 0.67 | 1.225 |
| 33 | 4Pent | 546.2 | 6.632 | 1.38 | 2.01 |
| 34 | 4Pent | 546.2 | 10.927 | 0.55 | 3.311 |
| 35 | 4Pent | 546.2 | 10.982 | 3.69 | 3.328 |
| 36 | 4Pent | 546.2 | 11.359 | 1.02 | 3.442 |
| 37 | 4Pent | 546.2 | 11.712 | 3.62 | 3.549 |
| 38 | 4Pent | 546.2 | 12.493 | 2.06 | 3.786 |
| 39 | 4Pent | 546.2 | 12.778 | 1.86 | 3.872 |
| 40 | 4Pent | 546.2 | 13.301 | 2.3 | 4.031 |
| 41 | 4Pent | 546.2 | 14.03 | 2.07 | 4.252 |
| 42 | 4Pent | 546.2 | 16.028 | 3.61 | 4.857 |
| 43 | 4Pent | 546.2 | 16.64 | 1.37 | 5.042 |
| 44 | 5Hex | 828.29 | 11.06 | 0.88 | 3.352 |
| 45 | 5Pent | 678.24 | 15.339 | 0.92 | 4.648 |
| 46 | 5Pent | 678.24 | 15.787 | 1.36 | 4.784 |
| 47 | 5Pent | 678.24 | 16.082 | 2.71 | 4.873 |
| 48 | 5Pent | 678.24 | 16.642 | 1.43 | 5.043 |
| 49 | 5Pent | 678.24 | 17.207 | 0.69 | 5.214 |
| 50 | 5Pent | 678.24 | 17.957 | 1.83 | 5.442 |
| 51 | 5Pent | 678.24 | 20.845 | 0.48 | 6.317 |
| 52 | 6Hex | 990.34 | 15.36 | 0.57 | 4.655 |
| 53 | 6Pent | 810.28 | 17.546 | 0.42 | 5.317 |
| 54 | 6Pent | 810.28 | 19.118 | 0.93 | 5.793 |
| 55 | 6Pent | 810.28 | 19.47 | 0.99 | 5.9 |

TABLE V
CLX 123 comprises the oligosaccharides shown in
this table (Pent = pentose, generally arabinose,
and Hex = hexose, generally glucose or mannose).

| | | | | | Reten- |
|---|---|---|---|---|---|
| Com- | | | RT | Oligo | tion |
| pound | Identity | Mass | (min) | Wt. % | Factor |
| 1 | 2Hex1HexA | 518.16 | 8.325 | 0.62 | 4.73 |
| 2 | 2Hex1HexA | 518.16 | 8.947 | 3.64 | 5.084 |
| 3 | 2Hex1Pent | 474.17 | 2.959 | 0.17 | 1.681 |
| 4 | 2Hex1Pent | 474.17 | 3.194 | 1.02 | 1.815 |
| 5 | 2Hex1Pent | 474.17 | 4.957 | 3.69 | 2.816 |
| 6 | 2Hex1Pent | 474.17 | 6.228 | 0.45 | 3.539 |
| 7 | 2Hex1Pent | 474.17 | 7.324 | 0.47 | 4.161 |
| 8 | 2Hex1Pent | 474.17 | 8.744 | 0.56 | 4.968 |
| 9 | 3Hex | 504.18 | 1.76 | 0.23 | 1 |
| 10 | 3Hex | 504.18 | 2.979 | 1.35 | 1.693 |
| 11 | 3Hex | 504.18 | 3.493 | 3.1 | 1.985 |
| 12 | 3Hex | 504.18 | 4.368 | 0.71 | 2.482 |
| 13 | 3Hex | 504.18 | 4.621 | 5.04 | 2.626 |
| 14 | 3Hex | 504.19 | 4.969 | 10.34 | 2.823 |
| 15 | 3Hex | 504.18 | 6.283 | 1.47 | 3.57 |
| 16 | 3Hex | 504.18 | 7.081 | 1.01 | 4.023 |
| 17 | 3Hex | 504.18 | 9.241 | 1.5 | 5.251 |
| 18 | 3Hex | 504.18 | 9.776 | 2.12 | 5.555 |
| 19 | 3Hex | 504.18 | 15.365 | 1.46 | 8.73 |
| 20 | 3Hex1HexA | 680.22 | 4.082 | 0.5 | 2.319 |
| 21 | 3Hex1HexA | 680.22 | 6.239 | 0.48 | 3.545 |
| 22 | 3Hex1HexA | 680.22 | 19.397 | 2.04 | 11.021 |
| 23 | 3Hex1Pent | 636.23 | 7.418 | 0.22 | 4.215 |
| 24 | 3Hex1Pent | 636.23 | 8.142 | 3.1 | 4.626 |
| 25 | 3Hex1Pent | 636.23 | 9.592 | 1.44 | 5.45 |
| 26 | 3Hex1Pent | 636.23 | 10.399 | 1.06 | 5.909 |
| 27 | 3Hex1Pent | 636.23 | 10.963 | 1.67 | 6.229 |
| 28 | 3Hex1Pent | 636.23 | 16.021 | 0.65 | 9.103 |
| 29 | 4Hex | 666.24 | 7.534 | 1.63 | 4.281 |
| 30 | 4Hex | 666.24 | 7.918 | 0.6 | 4.499 |
| 31 | 4Hex | 666.24 | 8.781 | 7.65 | 4.989 |
| 32 | 4Hex | 666.24 | 9.533 | 3.13 | 5.416 |
| 33 | 4Hex | 666.24 | 10.45 | 4.42 | 5.938 |
| 34 | 4Hex | 666.24 | 10.889 | 3.98 | 6.187 |
| 35 | 4Hex | 666.24 | 11.44 | 0.5 | 6.5 |
| 36 | 4Hex | 666.24 | 11.7 | 0.56 | 6.648 |
| 37 | 4Hex | 666.24 | 12.426 | 1.56 | 7.06 |
| 38 | 4Hex | 666.24 | 16.009 | 0.45 | 9.096 |
| 39 | 4Hex | 666.24 | 16.252 | 1.35 | 9.234 |
| 40 | 4Hex | 666.24 | 25.297 | 0.23 | 14.373 |
| 41 | 4Hex1HexA | 842.27 | 8.233 | 0.65 | 4.678 |
| 42 | 4Hex1HexA | 842.27 | 13.575 | 1.24 | 7.713 |
| 43 | 4Hex1Pent | 798.28 | 11.859 | 0.58 | 6.738 |
| 44 | 4Hex1Pent | 798.28 | 12.178 | 0.74 | 6.919 |
| 45 | 4Hex1Pent | 798.28 | 13.095 | 2.49 | 7.44 |
| 46 | 4Hex1Pent | 798.28 | 14.144 | 0.79 | 8.036 |
| 47 | 4Hex1Pent | 798.28 | 19.536 | 0.42 | 11.1 |
| 48 | 5Hex | 828.29 | 11.298 | 0.55 | 6.419 |
| 49 | 5Hex | 828.29 | 11.761 | 0.51 | 6.682 |
| 50 | 5Hex | 828.29 | 11.962 | 2.04 | 6.797 |
| 51 | 5Hex | 828.29 | 12.608 | 1.79 | 7.164 |
| 52 | 5Hex | 828.29 | 12.853 | 5.08 | 7.303 |
| 53 | 5Hex | 828.29 | 13.744 | 0.63 | 7.809 |
| 54 | 5Hex | 828.29 | 14.038 | 2.43 | 7.976 |
| 55 | 5Hex | 828.29 | 15.144 | 0.39 | 8.605 |
| 56 | 5Hex | 828.29 | 17.108 | 0.41 | 9.72 |
| 57 | 5Hex | 828.29 | 19.354 | 0.82 | 10.997 |
| 58 | 5Hex | 828.29 | 21.535 | 0.24 | 12.236 |
| 59 | 5Hex1Pent | 960.33 | 15.57 | 0.28 | 8.847 |
| 60 | 6Hex | 990.34 | 15.31 | 0.93 | 8.699 |
| 61 | 7Hex | 1152.39 | 16.851 | 0.77 | 9.574 |

TABLE W
CLX 124 comprises the oligosaccharides shown in this table.

| | | | | | Reten- |
|---|---|---|---|---|---|
| Com- | | | RT | Oligo | tion |
| pound | Identity | Mass | (min) | Wt. % | Factor |
| 1 | 1Hex2Pent | 444.16 | 3.49 | 2.79 | 2.165 |
| 2 | 1Hex2Pent | 444.16 | 4.245 | 0.86 | 2.633 |
| 3 | 2Hex1Pent | 474.17 | 2.556 | 6.88 | 1.586 |
| 4 | 2Hex1Pent | 474.18 | 3.526 | 0.98 | 2.187 |
| 5 | 2Hex2Pent | 606.22 | 9.748 | 1.92 | 6.047 |
| 6 | 3Hex | 504.18 | 1.612 | 7.96 | 1 |
| 7 | 3Hex | 504.18 | 1.893 | 1.51 | 1.174 |
| 8 | 3Hex | 504.18 | 2.501 | 0.61 | 1.551 |
| 9 | 3Hex | 504.18 | 2.914 | 1.51 | 1.808 |
| 10 | 3Hex | 504.18 | 6.515 | 3.95 | 4.042 |
| 11 | 3Hex | 504.18 | 15.334 | 0.51 | 9.512 |
| 12 | 3Hex (NR) | 504.17 | 3.977 | 3.31 | 2.467 |
| 13 | 3Hex1Pent | 636.23 | 7.337 | 11.63 | 4.551 |
| 14 | 3Hex1Pent | 636.23 | 7.99 | 0.32 | 4.957 |
| 15 | 3Hex1Pent | 636.23 | 11.063 | 0.67 | 6.863 |
| 16 | 3Hex1Pent | 636.23 | 12.762 | 0.52 | 7.917 |
| 17 | 3Hex1Pent | 636.23 | 17.198 | 0.35 | 10.669 |
| 18 | 3Hex1Pent | 636.22 | 18.732 | 0.33 | 11.62 |
| 19 | 3Hex2Pent | 768.27 | 12.108 | 1.25 | 7.511 |
| 20 | 4Hex | 666.24 | 4.466 | 16.37 | 2.77 |
| 21 | 4Hex | 666.24 | 6.189 | 0.36 | 3.839 |
| 22 | 4Hex | 666.24 | 7.072 | 1.02 | 4.387 |
| 23 | 4Hex | 666.24 | 7.779 | 1.14 | 4.826 |
| 24 | 4Hex | 666.24 | 10.083 | 1.37 | 6.255 |
| 25 | 4Hex | 666.24 | 11.392 | 0.63 | 7.067 |
| 26 | 4Hex | 666.24 | 16.026 | 1.28 | 9.942 |
| 27 | 4Hex | 666.24 | 17.584 | 0.38 | 10.908 |
| 28 | 4Hex | 666.24 | 25.259 | 0.41 | 15.669 |
| 29 | 4Hex1Pent | 798.28 | 10.802 | 8.61 | 6.701 |
| 30 | 4Hex1Pent | 798.28 | 17.816 | 0.49 | 11.052 |
| 31 | 5Hex | 828.29 | 8.774 | 2.19 | 5.443 |
| 32 | 5Hex | 828.29 | 16.339 | 1.0 | 10.136 |
| 33 | 5Hex | 828.29 | 22.876 | 0.72 | 14.191 |
| 34 | 5Hex1Pent | 960.33 | 13.533 | 4.45 | 8.395 |
| 35 | 6Hex | 990.34 | 12.239 | 6.61 | 7.592 |
| 36 | 6Hex | 990.34 | 13.162 | 0.5 | 8.165 |
| 37 | 6Hex1Pent | 1122.38 | 15.771 | 1.91 | 9.783 |
| 38 | 7Hex | 1152.39 | 14.381 | 2.7 | 8.921 |

TABLE X
CLX 125 comprises the oligosaccharides shown in this table.

| | | | | | Reten- |
|---|---|---|---|---|---|
| Com- | | | RT | Oligo | tion |
| pound | Identity | Mass | (min) | Wt. % | Factor |
| 1 | 1Hex1HexA1Deoxyhex | 502.17 | 19.054 | 10.75 | 7.46 |
| 2 | 2Hex1Deoxyhex | 488.19 | 2.554 | 5.22 | 1 |
| 3 | 2Hex1HexA | 518.16 | 18.245 | 6.09 | 7.144 |
| 4 | 2Hex1HexA1Deoxyhex | 664.22 | 17.01 | 20.06 | 6.66 |
| 5 | 2Hex1HexA1Deoxyhex | 664.22 | 26.202 | 11.78 | 10.259 |
| 6 | 2Hex1HexA1Deoxyhex | 664.22 | 30.461 | 6.73 | 11.927 |
| 7 | 2Hex1HexA2Deoxyhex | 810.28 | 34.83 | 7.10 | 13.637 |
| 8 | 3Hex | 504.19 | 3.133 | 0.21 | 1.227 |
| 9 | 3Hex | 504.19 | 3.384 | 0.24 | 1.325 |
| 10 | 3Hex | 504.18 | 4.804 | 2.33 | 1.881 |
| 11 | 3Hex1HexA1Deoxyhex | 826.28 | 28.498 | 11.38 | 11.158 |
| 12 | 3Hex1HexA1Deoxyhex | 826.27 | 28.523 | 3.44 | 11.168 |
| 13 | 3Hex1HexA2Deoxyhex | 972.33 | 32.07 | 8.64 | 12.557 |
| 14 | 4Hex | 666.24 | 9.489 | 0.27 | 3.715 |
| 15 | 4Hex | 666.24 | 10.444 | 0.32 | 4.089 |
| 16 | 4Hex | 666.24 | 10.847 | 2.00 | 4.247 |
| 17 | 4Hex | 666.23 | 16.998 | 1.52 | 6.655 |
| 18 | 5Hex | 828.29 | 13.948 | 1.36 | 5.461 |
| 19 | 8Hex | 1314.46 | 26.197 | 0.55 | 10.257 |

TABLE Y
CLX 126 comprises the oligosaccharides shown in this
table (Hex = hexose, generally glucose or galactose,
Pent = pentose, generally arabinose or xylose).

| | | | | | Reten- |
|---|---|---|---|---|---|
| Com- | | | RT | Oligo | tion |
| pound | Identity | Mass | (min) | Wt. % | Factor |
| 1 | 2Hex1HexA | 518.16 | 5.195 | 2.97 | 1.612 |
| 2 | 2Hex1Pent | 474.17 | 3.223 | 2.41 | 1 |
| 3 | 2Hex1Pent | 474.17 | 3.759 | 8.13 | 1.166 |
| 4 | 2Hex2Pent | 606.22 | 8.304 | 1.36 | 2.576 |
| 5 | 2Hex2Pent | 606.22 | 9.147 | 2.01 | 2.838 |
| 6 | 3Hex | 504.18 | 3.24 | 8.55 | 1.005 |
| 7 | 3Hex | 504.18 | 5.024 | 1.54 | 1.559 |
| 8 | 3Hex1HexA | 680.22 | 10.517 | 5.15 | 3.263 |
| 9 | 3Hex1Pent | 636.23 | 7.456 | 0.70 | 2.313 |
| 10 | 3Hex1Pent | 636.22 | 8.003 | 0.83 | 2.483 |
| 11 | 3Hex1Pent | 636.23 | 8.309 | 2.00 | 2.578 |
| 12 | 3Hex1Pent | 636.23 | 9.023 | 9.78 | 2.8 |
| 13 | 3Hex2Pent | 768.27 | 11.553 | 0.98 | 3.585 |
| 14 | 3Hex2Pent | 768.27 | 11.749 | 0.64 | 3.645 |
| 15 | 3Hex2Pent | 768.27 | 12.553 | 1.84 | 3.895 |
| 16 | 4Hex | 666.24 | 8.283 | 9.35 | 2.57 |
| 17 | 4Hex | 666.24 | 10.913 | 1.47 | 3.386 |
| 18 | 4Hex | 666.24 | 19.608 | 0.38 | 6.084 |
| 19 | 4Hex1HexA | 842.27 | 13.588 | 4.66 | 4.216 |
| 20 | 4Hex1Pent | 798.28 | 10.957 | 0.49 | 3.4 |
| 21 | 4Hex1Pent | 798.28 | 11.271 | 1.01 | 3.497 |
| 22 | 4Hex1Pent | 798.28 | 12.045 | 9.22 | 3.737 |
| 23 | 4Hex2Pent | 930.32 | 13.503 | 0.42 | 4.19 |
| 24 | 5Hex | 828.29 | 11.557 | 6.93 | 3.586 |
| 25 | 5Hex1HexA | 1004.32 | 15.435 | 3.39 | 4.789 |
| 26 | 5Hex1Pent | 960.33 | 13.319 | 0.89 | 4.132 |
| 27 | 5Hex1Pent | 960.33 | 13.904 | 5.94 | 4.314 |
| 28 | 6Hex | 990.34 | 13.511 | 4.43 | 4.192 |
| 29 | 6Hex1HexA | 1166.38 | 16.633 | 1.45 | 5.161 |
| 30 | 7Hex1Pent | 1284.44 | 16.31 | 1.09 | 5.061 |

TABLE Z
CLX 127 comprises the oligosaccharides shown in this
table (Hex = hexose, generally galactose or
glucose, HexA = glucuronic acid or galacturonic acid).

| | | | | | Reten- |
|---|---|---|---|---|---|
| Com- | | | RT | Oligo | tion |
| pound | Identity | Mass | (min) | Wt. % | Factor |
| 1 | 2Hex1HexA | 518.16 | 4.847 | 4.08 | 1.542 |
| 2 | 2Hex1Pent | 474.17 | 3.158 | 3.15 | 1.004 |
| 3 | 2Hex1Pent | 474.17 | 3.655 | 6.39 | 1.163 |
| 4 | 2Hex2Pent | 606.22 | 9.104 | 1.54 | 2.896 |
| 5 | 3Hex | 504.19 | 3.144 | 13.45 | 1 |
| 6 | 3Hex | 504.18 | 4.11 | 0.64 | 1.307 |
| 7 | 3Hex | 504.19 | 4.746 | 1.17 | 1.51 |
| 8 | 3Hex | 504.18 | 8.699 | 0.7 | 2.767 |
| 9 | 3Hex | 504.18 | 10.063 | 1.05 | 3.201 |
| 10 | 3Hex | 504.18 | 15.314 | 1.75 | 4.871 |
| 11 | 3Hex1HexA | 680.22 | 10.322 | 3.86 | 3.283 |
| 12 | 3Hex1Pent | 636.23 | 8.218 | 3.22 | 2.614 |
| 13 | 3Hex1Pent | 636.23 | 8.969 | 6.63 | 2.853 |
| 14 | 3Hex2Pent | 768.27 | 12.501 | 1.43 | 3.976 |
| 15 | 4Hex | 666.24 | 8.181 | 13.39 | 2.602 |
| 16 | 4Hex | 666.24 | 9.36 | 0.56 | 2.977 |
| 17 | 4Hex | 666.24 | 10.786 | 1.12 | 3.431 |
| 18 | 4Hex | 666.24 | 19.573 | 0.65 | 6.226 |
| 19 | 4Hex | 666.24 | 20.122 | 0.56 | 6.4 |
| 20 | 4Hex | 666.24 | 25.25 | 1.12 | 8.031 |
| 21 | 4Hex1HexA | 842.27 | 13.425 | 2.57 | 4.27 |
| 22 | 4Hex1Pent | 798.28 | 11.989 | 7.33 | 3.813 |
| 23 | 4Hex2Pent | 930.32 | 14.282 | 0.84 | 4.543 |
| 24 | 5Hex | 828.29 | 11.489 | 9.34 | 3.654 |
| 25 | 5Hex | 828.29 | 12.209 | 0.4 | 3.883 |
| 26 | 5Hex | 828.29 | 26.348 | 0.39 | 8.38 |
| 27 | 5Hex | 828.29 | 27.127 | 0.31 | 8.628 |
| 28 | 5Hex | 828.29 | 29.233 | 0.75 | 9.298 |
| 29 | 5Hex1Pent | 960.33 | 13.887 | 3.62 | 4.417 |
| 30 | 6Hex | 990.34 | 13.482 | 4.8 | 4.288 |
| 31 | 6Hex1Pent | 1122.38 | 15.142 | 1.9 | 4.816 |
| 32 | 7Hex | 1152.39 | 14.813 | 0.62 | 4.712 |
| 33 | 8Hex | 1314.45 | 15.9 | 0.66 | 5.057 |

TABLE AA
CLX 128 comprises the oligosaccharides shown in this
table (Hex = hexose, generally glucose or galactose,
Pent = pentose, generally xylose or arabinose).

| | | | | | Reten- |
|---|---|---|---|---|---|
| Com- | | | RT | Oligo | tion |
| pound | Identity | Mass | (min) | Wt. % | Factor |
| 1 | 2Hex1Pent | 474.18 | 13.955 | 3.91 | 7.131 |
| 2 | 3Hex | 504.18 | 11.599 | 1.85 | 5.927 |
| 3 | 3Hex | 504.19 | 15.414 | 18.53 | 7.876 |
| 4 | 3Hex | 504.18 | 18.158 | 2.07 | 9.278 |
| 5 | 3Hex (NR) | 504.17 | 1.957 | 1.30 | 1 |
| 6 | 3Hex (NR) | 504.17 | 3.84 | 0.30 | 1.962 |
| 7 | 3Hex (NR) | 504.17 | 5.404 | 1.29 | 2.761 |
| 8 | 3Hex1Pent | 636.23 | 24.526 | 2.88 | 12.532 |
| 9 | 3Hex1Pent | 636.23 | 25.874 | 0.31 | 13.221 |
| 10 | 3Pent | 414.15 | 2.398 | 0.39 | 1.225 |
| 11 | 3Pent | 414.15 | 9.736 | 8.48 | 4.975 |
| 12 | 3Pent | 414.15 | 13.843 | 0.82 | 7.074 |
| 13 | 4Hex | 666.24 | 21.972 | 1.80 | 11.227 |
| 14 | 4Hex | 666.24 | 25.37 | 13.78 | 12.964 |
| 15 | 4Hex | 666.24 | 25.62 | 2.34 | 13.091 |
| 16 | 4Hex | 666.24 | 26.463 | 2.28 | 13.522 |
| 17 | 4Hex1Pent | 798.28 | 29.11 | 1.70 | 14.875 |
| 18 | 4Pent | 546.19 | 10.029 | 1.13 | 5.125 |
| 19 | 4Pent | 546.2 | 15.641 | 1.48 | 7.992 |
| 20 | 4Pent | 546.19 | 18.132 | 7.52 | 9.265 |
| 21 | 4Pent | 546.2 | 21.384 | 0.76 | 10.927 |
| 22 | 5Hex | 828.29 | 28.523 | 0.74 | 14.575 |
| 23 | 5Hex | 828.29 | 29.329 | 8.52 | 14.987 |
| 24 | 5Hex | 828.29 | 29.834 | 1.76 | 15.245 |
| 25 | 5Hex | 828.29 | 30.473 | 2.21 | 15.571 |
| 26 | 5Pent | 678.24 | 17.349 | 0.70 | 8.865 |
| 27 | 5Pent | 678.24 | 20.908 | 0.79 | 10.684 |
| 28 | 5Pent | 678.24 | 21.722 | 1.17 | 11.1 |
| 29 | 5Pent | 678.24 | 24.34 | 5.23 | 12.437 |
| 30 | 5Pent | 678.24 | 26.576 | 0.50 | 13.58 |
| 31 | 6Hex | 990.34 | 33.38 | 0.47 | 17.057 |
| 32 | 6Pent | 810.28 | 27.583 | 1.49 | 14.095 |
| 33 | 7Pent | 942.32 | 29.641 | 1.51 | 15.146 |

TABLE BB
CLX 129 comprises the oligosaccharides shown in this table.

| | | | | | Reten- |
|---|---|---|---|---|---|
| Com- | | | RT | Oligo | tion |
| pound | Identity | Mass | (min) | Wt. % | Factor |
| 1 | 3Hex | 504.19 | 10.311 | 1.54 | 1 |
| 2 | 3Hex | 504.19 | 11.17 | 57.93 | 1.083 |
| 3 | 3Hex | 504.19 | 11.66 | 1.76 | 1.131 |
| 4 | 3Hex | 504.19 | 13.234 | 7.75 | 1.283 |
| 5 | 4Hex | 666.24 | 15.979 | 16.91 | 1.55 |
| 6 | 4Hex | 666.24 | 24.101 | 1.69 | 2.337 |
| 7 | 4Hex | 666.24 | 25.316 | 5.69 | 2.455 |
| 8 | 5Hex | 828.29 | 20.596 | 3.7 | 1.997 |
| 9 | 5Hex | 828.29 | 30.229 | 3.02 | 2.932 |

TABLE CC
CLX 132 comprises the oligosaccharides shown in this table.

| | | | | | Reten- |
|---|---|---|---|---|---|
| Com- | | | RT | Oligo | tion |
| pound | Identity | Mass | (min) | Wt. % | Factor |
| 1 | 1Hex2Pent | 444.16 | 3.718 | 0.75 | 1.172 |
| 2 | 1Hex2Pent | 444.16 | 4.377 | 0.83 | 1.379 |
| 3 | 1Hex2Pent | 444.16 | 4.632 | 1.51 | 1.46 |
| 4 | 1Hex2Pent | 444.16 | 12.001 | 0.29 | 3.782 |
| 5 | 2Hex1Pent | 474.17 | 3.188 | 0.49 | 1.005 |
| 6 | 2Hex1Pent | 474.17 | 3.709 | 7.77 | 1.169 |
| 7 | 2Hex1Pent | 474.17 | 3.716 | 0.53 | 1.171 |
| 8 | 2Hex1Pent | 474.17 | 4.815 | 5.84 | 1.517 |
| 9 | 2Hex1Pent | 474.17 | 10.775 | 0.97 | 3.396 |
| 10 | 2Hex2Pent | 606.22 | 9.138 | 0.77 | 2.88 |
| 11 | 2Hex2Pent | 606.21 | 9.924 | 0.41 | 3.128 |
| 12 | 2Hex2Pent | 606.21 | 15.467 | 0.56 | 4.875 |
| 13 | 2Hex2Pent | 606.22 | 16.12 | 0.47 | 5.08 |
| 14 | 2Hex2Pent | 606.22 | 16.855 | 0.82 | 5.312 |
| 15 | 2Hex2Pent | 606.22 | 18.948 | 0.19 | 5.972 |
| 16 | 3Hex | 504.18 | 3.173 | 6.87 | 1 |
| 17 | 3Hex | 504.18 | 4.188 | 0.21 | 1.32 |
| 18 | 3Hex | 504.18 | 4.815 | 0.89 | 1.517 |
| 19 | 3Hex | 504.18 | 6.169 | 5.69 | 1.944 |
| 20 | 3Hex (NR) | 504.17 | 9.422 | 0.72 | 2.969 |
| 21 | 3Hex1Pent | 636.23 | 7.423 | 7.34 | 2.339 |
| 22 | 3Hex1Pent | 636.23 | 8.283 | 0.53 | 2.61 |
| 23 | 3Hex1Pent | 636.23 | 8.997 | 9.54 | 2.835 |
| 24 | 3Hex1Pent | 636.23 | 10.878 | 0.53 | 3.428 |
| 25 | 3Hex1Pent | 636.23 | 17.498 | 0.85 | 5.515 |
| 26 | 3Hex1Pent | 636.23 | 18.194 | 0.3 | 5.734 |
| 27 | 3Hex1Pent | 636.23 | 18.542 | 0.3 | 5.844 |
| 28 | 3Hex2Pent | 768.27 | 12.532 | 0.88 | 3.95 |
| 29 | 3Hex2Pent | 768.27 | 15.417 | 0.89 | 4.859 |
| 30 | 3Hex2Pent | 768.27 | 19.189 | 0.72 | 6.048 |
| 31 | 3Hex2Pent | 768.27 | 21.439 | 0.24 | 6.757 |
| 32 | 4Hex | 666.24 | 6.403 | 4.75 | 2.018 |
| 33 | 4Hex | 666.24 | 8.239 | 6.94 | 2.597 |
| 34 | 4Hex | 666.24 | 10.798 | 1.04 | 3.403 |
| 35 | 4Hex | 666.24 | 17.659 | 0.32 | 5.565 |
| 36 | 4Hex1Pent | 798.28 | 11.58 | 3.05 | 3.65 |
| 37 | 4Hex1Pent | 798.28 | 12.022 | 7.07 | 3.789 |
| 38 | 4Hex1Pent | 798.28 | 13.995 | 0.52 | 4.411 |
| 39 | 4Hex1Pent | 798.28 | 14.492 | 0.65 | 4.567 |
| 40 | 5Hex | 828.29 | 10.988 | 1.62 | 3.463 |
| 41 | 5Hex | 828.29 | 11.508 | 4.91 | 3.627 |
| 42 | 5Hex1Pent | 960.33 | 13.873 | 4.53 | 4.372 |
| 43 | 5Hex1Pent | 960.33 | 15.799 | 0.25 | 4.979 |
| 44 | 6Hex | 990.34 | 13.474 | 2.96 | 4.246 |
| 45 | 6Hex1Pent | 1122.38 | 15.097 | 2.23 | 4.758 |
| 46 | 7Hex | 1152.39 | 14.813 | 0.26 | 4.668 |
| 47 | 7Hex | 1152.39 | 16.921 | 0.23 | 5.333 |

TABLE DD
CLX 133 comprises the oligosaccharides shown in this table
(Hex = hexose, generally galactose or glucose, Pent = pentose,
generally arabinose or xylose, Deoxy = Rhamnose or Fucose).

| | | | | | Reten- |
|---|---|---|---|---|---|
| Com- | | | RT | Oligo | tion |
| pound | Identity | Mass | (min) | Wt. % | Factor |
| 1 | 1Hex1Pent1Deoxy | 458.18 | 13.753 | 9.1 | 9.369 |
| 2 | 1Hex2Pent | 444.17 | 7.755 | 0.62 | 5.283 |
| 3 | 2Hex1Pent | 474.17 | 2.192 | 0.36 | 1.493 |
| 4 | 2Hex1Pent | 474.17 | 3.018 | 2.38 | 2.056 |
| 5 | 2Hex1Pent | 474.17 | 3.925 | 1.0 | 2.674 |
| 6 | 2Hex1Pent | 474.17 | 6.7 | 2.53 | 4.564 |
| 7 | 2Hex1Pent | 474.17 | 7.217 | 3.35 | 4.916 |
| 8 | 2Hex1Pent | 474.17 | 9.613 | 0.74 | 6.548 |
| 9 | 2Hex1Pent | 474.17 | 10.081 | 0.93 | 6.867 |
| 10 | 2Hex1Pent | 474.17 | 10.287 | 6.07 | 7.007 |
| 11 | 2Hex1Pent | 474.17 | 11.209 | 1.16 | 7.636 |
| 12 | 2Hex2Pent | 606.22 | 14.538 | 4.69 | 9.903 |
| 13 | 3Hex | 504.18 | 1.468 | 0.95 | 1 |
| 14 | 3Hex | 504.18 | 2.139 | 0.35 | 1.457 |
| 15 | 3Hex | 504.18 | 2.626 | 3.54 | 1.789 |
| 16 | 3Hex | 504.19 | 2.861 | 0.49 | 1.949 |
| 17 | 3Hex | 504.18 | 3.918 | 2.44 | 2.669 |
| 18 | 3Hex | 504.18 | 7.895 | 1.28 | 5.378 |
| 19 | 3Hex | 504.18 | 9.287 | 2.82 | 6.326 |
| 20 | 3Hex | 504.18 | 13.517 | 0.71 | 9.208 |
| 21 | 3Hex | 504.18 | 14.719 | 5.66 | 10.027 |
| 22 | 3Hex1Pent | 636.23 | 7.914 | 1.48 | 5.391 |
| 23 | 3Hex1Pent | 636.23 | 12.155 | 0.72 | 8.28 |
| 24 | 3Hex1Pent | 636.23 | 13.286 | 0.99 | 9.05 |
| 25 | 3Hex1Pent | 636.23 | 18.281 | 0.96 | 12.453 |
| 26 | 3Hex1Pent | 636.23 | 19.503 | 0.43 | 13.285 |
| 27 | 3Hex1Pent | 636.23 | 19.974 | 1.33 | 13.606 |
| 28 | 3Hex1Pent | 636.23 | 21.372 | 1.5 | 14.559 |
| 29 | 3Hex2Pent | 768.27 | 16.654 | 0.96 | 11.345 |
| 30 | 3Hex2Pent | 768.27 | 17.425 | 2.14 | 11.87 |
| 31 | 3Hex2Pent | 768.27 | 22.775 | 1.23 | 15.514 |
| 32 | 3Hex2Pent | 768.27 | 23.886 | 1.76 | 16.271 |
| 33 | 3Hex2Pent | 768.27 | 24.76 | 1.55 | 16.866 |
| 34 | 3Hex3Pent | 900.31 | 26.171 | 0.64 | 17.828 |
| 35 | 3Pent | 414.15 | 8.896 | 1.71 | 6.06 |
| 36 | 4Hex | 666.24 | 3.813 | 0.34 | 2.597 |
| 37 | 4Hex | 666.24 | 6.154 | 0.65 | 4.192 |
| 38 | 4Hex | 666.24 | 7.092 | 1.63 | 4.831 |
| 39 | 4Hex | 666.24 | 8.621 | 0.52 | 5.873 |
| 40 | 4Hex | 666.24 | 9.904 | 1.71 | 6.747 |
| 41 | 4Hex | 666.24 | 11.461 | 0.84 | 7.807 |
| 42 | 4Hex | 666.24 | 12.074 | 1.44 | 8.225 |
| 43 | 4Hex | 666.24 | 19.096 | 1.85 | 13.008 |
| 44 | 4Hex | 666.24 | 25.019 | 4.35 | 17.043 |
| 45 | 4Hex1Pent | 798.28 | 11.097 | 1.11 | 7.559 |
| 46 | 4Hex1Pent | 798.28 | 29.048 | 0.45 | 19.787 |
| 47 | 4Hex2Pent | 930.32 | 24.983 | 1.12 | 17.018 |
| 48 | 4Hex2Pent | 930.32 | 29.226 | 0.49 | 19.909 |
| 49 | 4Pent | 546.2 | 17.389 | 1.2 | 11.845 |
| 50 | 5Hex | 828.29 | 9.75 | 0.49 | 6.642 |
| 51 | 5Hex | 828.29 | 10.592 | 1.2 | 7.215 |
| 52 | 5Hex | 828.29 | 13.065 | 0.97 | 8.9 |
| 53 | 5Hex | 828.29 | 19.612 | 0.95 | 13.36 |
| 54 | 5Hex | 828.29 | 20.778 | 1.15 | 14.154 |
| 55 | 5Hex | 828.29 | 26.34 | 0.64 | 17.943 |
| 56 | 5Hex | 828.29 | 27.136 | 0.89 | 18.485 |
| 57 | 5Hex | 828.29 | 29.298 | 3.67 | 19.958 |
| 58 | 5Hex1Pent | 960.33 | 12.982 | 0.62 | 8.843 |
| 59 | 5Pent | 678.24 | 23.767 | 0.63 | 16.19 |
| 60 | 6Hex | 990.34 | 12.6 | 0.61 | 8.583 |
| 61 | 6Hex | 990.34 | 26.762 | 0.63 | 18.23 |
| 62 | 6Hex | 990.34 | 27.016 | 0.28 | 18.403 |
| 63 | 6Hex | 990.34 | 27.338 | 0.48 | 18.623 |
| 64 | 6Hex | 990.34 | 29.982 | 0.49 | 20.424 |

TABLE EE
CLX 116 comprises the oligosaccharides shown in
this table (Hex = hexose, generally galactose
or glucose, Pent = pentose, generally arabinose).

| | | | | | Reten- |
|---|---|---|---|---|---|
| Com- | | | RT | Oligo | tion |
| pound | Identity | Mass | (min) | Wt. % | Factor |
| 1 | 2Hex1Pent | 474.17 | 3.725 | 14.57 | 1.17 |
| 2 | 2Hex2Pent | 606.22 | 15.156 | 7.15 | 4.759 |
| 3 | 3Hex | 504.19 | 3.185 | 20.34 | 1 |
| 4 | 3Hex | 504.19 | 10.078 | 4.84 | 3.164 |
| 5 | 3Hex1Pent | 636.23 | 8.966 | 12.24 | 2.815 |
| 6 | 4Hex | 666.24 | 8.223 | 12.69 | 2.582 |
| 7 | 4Hex | 666.24 | 9.482 | 3.66 | 2.977 |
| 8 | 4Hex1Pent | 798.28 | 11.973 | 12.6 | 3.759 |
| 9 | 5Hex1Pent | 960.33 | 13.812 | 6.86 | 4.337 |
| 10 | 6Hex | 990.34 | 13.427 | 5.04 | 4.216 |

### DETAILED DESCRIPTION

In the following description, numerous specific details of the oligosaccharides, oligosaccharide compositions, and methods of the present invention are set forth in order to provide a thorough explanation of the precise nature of the invention. It will be apparent, however, to those of skill in the art that the invention can be practiced without these specific details. Although the following description is divided into sections, it is contemplated that each section contains various aspects of the invention and as such disclosure from within each section and across two or more sections can be combined to form any aspect of the invention.

### Methods to Generate Oligosaccharides and Oligosaccharide Compositions

In some aspects, the oligosaccharides and oligosaccharide compositions disclosed herein are produced by any suitable method or any combination of any method disclosed herein.

In some aspects, the oligosaccharides and oligosaccharide compositions can be produced by depolymerizing a suitable polysaccharide using a chemical method, such as oxidative chemistry.

In some aspects, the oligosaccharides and oligosaccharide compositions can be produced by a method comprising ground-up synthesis (e.g., biological synthesis or resin polymerization) by polymerizing monosaccharides and/or lower molecular weight oligosaccharides.

In some aspects, the oligosaccharides and oligosaccharide compositions can be created by elevated time, temperature, pressure processes.

In some aspects, the oligosaccharides and oligosaccharide compositions can be created by either depolymerization, polymerization, or transglycosylation by the use of enzymes. In some aspects, the one or more polysaccharide degrading enzyme(s) comprises, for example, an amylase, isoamylase, cellulase, maltase, glucanase, xylanase, lactase, or any combination thereof.

In some aspects, the oligosaccharides and oligosaccharide compositions can be created by chemical synthesis. In some aspects, the oligosaccharides can be synthesized in microorganisms such as yeast, algae, or bacteria, or any combination thereof. In some aspects, the oligosaccharides can be synthesized in eukaryotic cells.

In some aspects, the oligosaccharide composition can be created by depolymerization or polymerization by negative and/or positive solid state or soluble catalysts.

In some aspects, the oligosaccharide composition can be created from depolymerized from different natural products (e.g., polysaccharides found in nature). In some aspects, the natural product that the oligosaccharide composition was produced from does not matter, so long as the carbohydrate structure is similar. In some aspects, whether a carbohydrate composition was produced via depolymerization, polymerization, or transglycosylation does not matter, so long as the carbohydrate structure is similar.

In some aspects, the oligosaccharides and oligosaccharide compositions disclosed herein are produced from polysaccharides by a method known as Fenton's Initiation Toward Defined Oligosacharide Groups (FITDOG), as disclosed in WO 2018/236917, hereby incorporated by reference herein in its entirety for all purposes. Such methods comprise reacting a polysaccharide using Fenton's reagent composed of iron (Fe⁺, Fe²⁺) or other transition metal (including but not limited to, Cu¹⁺, Co²⁺, etc., including those disclosed herein for the COG method) and hydrogen peroxide. In some aspects, the reaction is allowed to proceed for 30 minutes (or for example, between 10 minutes and 4 hours, e.g., 15 minutes to 2 hours or 10 minutes to one hour). In some aspects, the transition metal or alkaline earth metal in the reaction mixture is at a concentration of at least 0.65 mM, or 0.65 mM to 20 mM, or 10 μM to 20 mM. The reaction is subsequently quenched with base (e.g., an Arrhenius base or strong Arrhenius base, such as aqueous sodium hydroxide calcium hydroxide, potassium hydroxide, etc., or any combination thereof). In some aspects, prior to the reacting, the method comprises contacting polysaccharides with one or more polysaccharide degrading enzyme, such as an amylase, isoamylase, cellulase, maltase, glucanase, or a combination thereof.

In some aspects, the oligosaccharides and oligosaccharide compositions disclosed herein are produced from polysaccharides by high-yield peroxide-quench-controlled methods (Controlled Oligosaccharide Generation (“COG”) methods), as disclosed in WO 2021/097138, hereby incorporated by reference in its entirety for all purposes. Such methods comprise a multi-step reaction (e.g., two-step, three-step, etc., reaction) that includes an initial oxidative step using a Fenton's system/reagent and a subsequent peroxide-quenching/PS-cleavage step using either: a PS-cleavage agent that also functions as a peroxide-quenching agent; or using a PS-cleavage agent in combination with a compatible peroxide-quenching reagent that does not interfere with the PS-cleavage reaction. In the methods, the PS-cleavage agent may be, for example, a weak-Arrhenius base or non-Arrhenius base. In the methods, the PS-cleavage initiator preferably also functions as a peroxide-quencher to quench (e.g., sufficiently reduce or eliminate) residual hydrogen peroxide and/or radicals thereof to minimize or eliminate off-target side reactions. The methods, for example, comprise reacting polysaccharides with hydrogen peroxide and a suitable metal or metal ion (e.g., a transition metal, alkaline earth metal, or lanthanide, such as, for example, Fe(II), Fe(III), Cu(I), Cu(II), Ca(II), Mg(II), Mn(II), Zn(II), Ni(II), Ce(IV), Co(II) or other metal ions, or any combination thereof), followed by cleaving glycosidic linkages in the hydroperoxyl-treated polysaccharides with a high-yield peroxide-quenching/cleavage agent such as ammonium bicarbonate, ammonium hydroxide, ammonia, urea, sodium amide, other ammonium-based reagent, a weak Arrhenius base, a non-Arrhenius base, a Lewis base, a Bronsted-Lowry base, or any combination thereof, thereby generating high yields of oligosaccharides, and lower molecular weight polysaccharides (polysaccharide cleavage products that are yet poly saccharides) from the parent (starting material) polysaccharides, while reducing or eliminating peeling (sequential alkaline degradation of carbohydrates through a mechanism that releases monomeric units from the reducing end of the polymer) and unwanted side-reactions.

In some aspects, the cleavage reagent may comprise at least one reagent selected from group consisting of ammonium hydroxide, ammonia, ammonium bicarbonate, urea, etc., or a combination thereof (e.g., see Table 1). In some aspects, the cleavage reagent may comprise the conjugate base of an alcohol or amine. In some aspects, the cleavage reagent may comprise sodium methoxide, sodium ethoxide, sodium tertbutoxide, or other deprotonated alcohol. In some aspects, the cleavage reagent may be or comprise one or more relatively “bulky bases” such as tert-butoxide, triethylamine, or other sterically hindered base. In some aspects, the use of such bulky cleavage reagents/bases results in selective cleavage of the accessible glycosidic bonds to provide oligosaccharide profiles unique/specific to the cleavage reagent/base. In some aspects the cleavage reagent is not a base, per se, but consists of, or comprises one or more reactive agent(s) that react to produce basic conditions and/or decomposition products. In all the methods described herein, the cleavage reagent (cleavage initiator) may also be, and preferably is a peroxide-quenching reagent, and in either case may be used in combination with an additional compatible peroxide-quenching agent that may or may not also be a cleavage agent.

In the disclosed COG methods, use of a peroxide-quencher to quench (e.g., sufficiently reduce or eliminate) residual hydrogen peroxide and/or radicals thereof per se, minimizes or eliminates off-target side reactions.

In the disclosed COG methods, use of particular weak Arrhenius bases and/or non-Arrhenius bases (e.g., ammonium-based peroxide-quenching/PS-cleavage reagents, etc.; e.g., see Table 1) not only provides for improved high-yield oligosaccharide production (relative to the strong Arrhenius bases used in the art), but also eliminates the need for costly and time-consuming post-reaction concentration, and desalting steps.

TABLE 1
Exemplary polysaccharide (PS)-cleavage,
and/or peroxide-quenching agents.

| | | |
|---|---|---|
| | Peroxide- | |
| Compound | quenching | Cleavage |

Exemplary nitrogen-based, peroxide-quenching, PS-cleavage agents

| | | |
|---|---|---|
| Ammonium Hydroxide | Yes | Yes |
| Ammonium | Yes | Yes |
| Bicarbonate | | |
| Ammonia | Yes | Yes |
| Urea | Yes | Yes |
| Sodium Amide | Yes | Yes |
| Trimethyl amine | (Yes) | Yes |
| Diethylamine | (Yes) | Yes |
| Pyridine | (Yes) | Yes |
| N,N- | (Yes) | Yes |
| Diisopropylethylamine | | |

Exemplary Lewis base, peroxide-quenching, PS-cleavage agents

| | | |
|---|---|---|
| F— | Yes | Yes |
| H— | Yes | Yes |
| Methoxide (CH₃O⁻) | Yes | Yes |
| Ethoxide (C₂H₅O⁻) | Yes | Yes |
| Tertbutoxide (C₄H₉O⁻) | Yes | Yes |

Exemplary strong Arrhenius base,
non-peroxide-quenching, PS-cleavage agents

| | | |
|---|---|---|
| Sodium Hydroxide | No | Yes |
| Potassium Hydroxide | No | Yes |
| Calcium Hydroxide | No | Yes |
| Cesium Hydroxide | No | Yes |
| Strontium Hydroxide | No | Yes |
| Lithium Hydroxide | No | Yes |
| Rubidium Hydroxide | No | Yes |

Exemplary peroxide-quenching, non-PS-cleavage agents

| | | |
|---|---|---|
| Methanol | Yes | No |
| Acetonitrile | Yes | No |
| Formaldehyde | Yes | No |
| Chlorine Gas | Yes | No |
| Salts of Sulfite | Yes | No |
| Salts of Thiosulfite | Yes | No |
| Catalase Enzyme | Yes | No |

In the methods, the cleavage initiator may, and preferably does, also function as a peroxide-quencher to quench (sufficiently reduce or eliminate) residual peroxide and/or radicals thereof to reduce or eliminate peeling and unwanted side-reactions. Alternatively, the high-yield cleavage agent can be added to the reaction after, or along with addition of a compatible peroxide-quenching agent (that could also be a cleavage reagent). In the methods, the peroxide-quenching/cleavage agent may be, and preferably is, selected from one or more nitrogen-based agents as described herein (e.g., see Table 1, above), and not only provides high-yield cleavage and residual peroxide-quenching, but also provides for cleavage specificity tailoring (e.g., by replacing nitrogen bound hydrogen with larger moieties to sterically hinder or otherwise modify access by, or activity of the cleavage agent).

In some aspects, the transition metal or alkaline earth metal in the reaction mixture is at a concentration of at least about 10 μM. In some aspects, the transition metal or alkaline earth metal in the reaction mixture is at a concentration of about 10 m to about 20 mM. In some aspects, the concentration is about at least about 0.65 mM (e.g. at least a value in the range of 0.5 to 0.7 mM). In some aspects, the transition metal or alkaline earth metal in the reaction mixture is at a concentration from 0.65 mM to 500 mM. In some aspects, the peroxide agent (e.g., hydrogen peroxide) in the reaction mixture is at a concentration of at least about 0.02 M (e.g. at least a value in the range of 0.015 to 0.025 M). In some aspects, the peroxide agent (e.g., hydrogen peroxide) in the reaction mixture is at a concentration of from 0.02 M to 1 M, or in some aspects up to 5 M. In some aspects, the peroxide agent (e.g., hydrogen peroxide) in the reaction mixture is at a concentration of from 1 M to 5 M. In some aspects, the cleavage reagent/base is or comprises ammonium hydroxide, ammonia, ammonium bicarbonate, a weak Arrhenius base, a non-Arrhenius base, a Lewis base, and/or a Bronsted-Lowry base. Moreover, combinations of two or more cleavage reagents/bases (e.g., such as the cleavage reagents/bases discussed herein) may be used. In some aspects, strong-Arrhenius bases (e.g., Na⁺OH⁻, K⁺OH⁻, or Ca⁺²(OH⁻)₂) can be used in combination with the cleavage reagents/bases discussed herein. In some aspects, ammonia gas can be in contact with the solution through bubbling or as an atmospheric component to act as a cleavage and/or quenching reagent. In some aspects, the cleavage reagent is at a concentration of at least about 0.1 M (+/−20%). In some aspects, the cleavage reagent is at a concentration of from 0.1 M-5.0 M. In some aspects the cleavage reagent is present as a saturated solution or insoluble material. In some aspects the cleavage reagent brings the solution to pH 7.5, 8, 9, 10, 12, or higher. In all the methods described herein, the cleavage reagent (cleavage initiator) may also be, and preferably is a peroxide-quenching reagent, and in either case may be used in combination with an additional compatible peroxide-quenching agent that may or may not also be a cleavage agent.

In some aspects, in the COG methods the crude polysaccharides first undergo initial oxidative treatment with the hydrogen peroxide and a transition metal, alkaline earth metal, or lanthanide catalyst to render the glycosidic linkages more labile. Ammonium hydroxide, ammonium bicarbonate, ammonia, urea, etc., or other weak Arrhenius or non-Arrhenius base is then used for cleavage, which results in a variety of distinctive oligosaccharides (distinctive oligosaccharide profile), or smaller polysaccharides. In some aspects, peroxide-quenching and/or neutralization takes place immediately to reduce unwanted oxidation, or peeling, respectively. In some aspects the treated sample (e.g., the polysaccharide comprising starting material after treatment with a Fenton's reagent) is allowed to react with the cleavage reagent at reduced, ambient, or room temperature to facilitate the production of oligosaccharides. In some aspects the cleavage reaction takes place at 4-100° C., 20-80° C., 30-60° C. or 40° C. In some aspects, cleavage and peroxide-quenching are immediate. In some aspects the cleavage step is conducted for 10-30 minutes, 20-60 minutes, 30-120 minutes. In some aspects the cleavage step is conducted for 2-6 hours, 3-12 hours, 6-24 hours or longer. In some aspects the cleavage step is conducted overnight. In all the methods described herein, the cleavage reagent (cleavage initiator) may also be, and preferably is a peroxide-quenching reagent, and in either case may be used in combination with an additional compatible peroxide-quenching agent that may or may not also be a cleavage agent. The disclosed COG methods have the ability to generate large amounts of biologically active oligosaccharides from a variety of carbohydrate sources (e.g., polysaccharide-containing starting materials).

In some aspects, the method of cleaving polysaccharides comprises multiple steps. For instance, the method can comprise: a) contacting one or more polysaccharide with a Fenton's reagent, comprising a peroxide agent and metal ions to form a mixture; b) allowing the Fenton's reagent to react with the polysaccharide for a specified reaction time; and c) after step b, adding a cleavage agent which may also be a peroxide quenching reagent to the mixture. In such aspect, the steps of contacting the polysaccharide with a Fenton's reagent (step a) and allowing a specified reaction time to pass (step b) can be performed at the same or different pH wherein the pH is selected from within a range of pH 3 to 8, pH 4 to 7, pH 4.5 to 6.5, and pH 5 to 6. The pH can be any possible value between the specified ranges of pH values. The step of adding a cleavage agent which may also be a peroxide quenching reagent (step c) can be performed at a pH selected from within a range of pH 6 to 11, pH 6.5 to 9.5, pH 7 to 9, and pH 7 to 8. The pH can be any possible value between the specified ranges of pH values. In such aspect, the step of contacting the polysaccharide with a Fenton's reagent (step a) and passage of the specified reaction time (step b) can be performed at the same or different temperature wherein the temperature is selected from within a range of temperature between 10 and 70 degrees Celsius, between 20 and 60 degrees Celsius, and between 25 and 55 degrees Celsius. The temperature can be any possible value between the specified ranges of temperature values. The step of adding a cleavage agent which may also be a peroxide quenching reagent (step c) can be performed at a temperature selected from within a range of temperature between 10 and 70 degrees Celsius, between 20 and 60 degrees Celsius, and between 25 and 55 degrees Celsius. The temperature can be any possible value between the specified ranges of temperature values.

In some aspects, if desired, the polysaccharide source material can optionally be treated with one or more polysaccharide-degrading enzyme(s) to reduce the average size or complexity of the polysaccharide before the resulting polysaccharides are treated with the COG or FITDOG methods. Non-limiting examples of polysaccharide enzymes include for example, amylase, isoamylase, cellulase, maltase, glucanase, lactase, xylanase, arabinase, pectinase, mannanase, or a combination thereof.

In some aspects, a method is provided for creating soluble fiber from insoluble fiber comprising polysaccharides using the COG or FITDOG reaction conditions described herein. By running the reaction only to a certain extent (e.g., partial depolymerization of the polysaccharide material), compositions having desirable characteristics (e.g., gels or salves) can be generated. The COG or FITDOG methods can be used to soften or alter the texture, porosity, or reaction properties of polysaccharide containing materials that are exposed (e.g., soaked, or permeated to some extent with) to the reaction constituents. In some aspects, the COG or FITDOG methods can be used to soften (e.g., by partial depolymerization) the cell wall of plants and/or plant materials, animals, bacteria, and fungi prior to industrial processing. In some aspects, softening the cell wall of plants may result in greater extractability of valuable components. In some aspects, softening the cell wall of plants or plant materials may result in easier physical removal or separation of wanted and/or unwanted parts (e.g., shells, skins, peels, seeds). In some aspects, the COG or FITDOG methods may be used to “soften” the cell wall of plants, bacteria, animals, and fungi to create permeable membranes prior to cellular modifications (e.g., nucleic acid (e.g., DNA and/or RNA) transfection and/or modification. In some aspects, the COG or FITDOG methods, or the one or more oligosaccharides or the oligosaccharide composition, can be used to alter the rheological properties of gums, gels, and other carbohydrate-derived textural/organoleptic modifiers. In some aspects, the COG or FITDOG methods can be used to produce smaller molecular weight carbohydrates and/or polysaccharides and/or oligosaccharides for the production of bio-ethanol, bio-fuel, or other downstream compounds.

In some aspects, the crude polysaccharides first undergo initial oxidative treatment with the hydrogen peroxide and a transition metal or alkaline earth metal (e.g., iron(III) sulfate) catalyst to render the glycosidic linkages more labile. A weak-Arrhenius base or non-Arrhenius base is then used for base induced cleavage, which results in a variety of oligosaccharides. Immediate neutralization may take place to reduce any peeling reaction. This method has the ability to generate large amounts of biologically active oligosaccharides from a variety of carbohydrate sources. The initial oxidative treatment can include hydrogen peroxide and a transition metal or an alkaline earth metal. Metals with different oxidation states, sizes, periodic groups, and coordination numbers have been tested to understand the application with the COG process. Each of the different metals has shown activity in the COG reaction. While these metals work with any polysaccharide, different metals can be used to produce oligosaccharides with preferential degrees of polymerization. The oxidative treatment is followed by a base treatment. The method is capable of generating oligosaccharides from polysaccharides having varying degrees of branching, and having a variety of monosaccharide compositions, including natural and modified polysaccharides.

### Source Polysaccharides and Source Materials Comprising Polysaccharides

Any suitable source polysaccharide, or source material comprising one or polysaccharides, can be used to prepare the oligosaccharides and oligosaccharide compositions disclosed herein. In some aspects, oligosaccharides and oligosaccharide compositions can be produced by suitably depolymerizing a polysaccharide obtained from a suitable source or combinations of suitable sources.

The methods disclosed herein, including the COG and FITDOG methods, are effective for producing bioactive oligosaccharides, and lower molecular weight polysaccharides, by digesting polysaccharides from any source, including but not limited to plants, bacteria, animals, algae, and fungi. In some aspects, the oligosaccharides are produced in the range of degree of polymerization (DP) of 3 to 20. In some aspects, polysaccharides are broken down to smaller polysaccharides. The methods disclosed herein, such as the COG and FITDOG methods, can be used to convert polysaccharides (e.g., from plants, bacteria, or yeast, algae, animals, fungi, and waste product streams) into bioactive oligosaccharides or smaller polysaccharides.

In some aspects, production from natural polysaccharide sources of oligosaccharides consisting of DP from 3 to 20 or 30 or more (or from 3 to up to 200 for example) is provided. The polysaccharides can include, for example, those from plants, algae, bacteria, animals, fungi, and waste product streams (e.g., food-waste product streams). In some aspects, the polysaccharides can come from food, agriculture, or biofuel waste products and from sources not usually considered food. In some aspects, the source of polysaccharide is processed foods, and plant products.

In some aspects, the oligosaccharides can be produced (e.g., having a DP between 3 and 20 (or from 3 to up to 200 for example)) from bacterial cell wall polysaccharides, yeast cell wall polysaccharides, algae polysaccharides, plant polysaccharides, or any combination thereof, optionally using the COG methods or any other method or combination of methods disclosed herein.

In some aspects, the oligosaccharide compositions are derived from natural products. In some aspects, those natural products are or comprise potato, icelandic moss, locust bean gum, Alcaligenes faecalis, larch, curdlan, microbial curdlan, konjac glucomannan, konjac, glucomannan, xylan, beechwood xylan, beechwood, arabinogalactan, galactan, potato pectic galactan, pectic galactan, lichenan, carob, carob galactomannan, galactomannan, beta glucan, barley beta glucan, oat beta glucan, barley, oat, xyloglucan, tamarind seed xyloglucan, tamarind seed, arabinoxylan, rye arabinoxylan, rye, Karaya gum, Saccharomyces cerevisiae, oranges, tragacanth gum, yeast, yeast derived beta glucan, tomato, pea, Xanthomonas Campestris, Xanthomonas Campestris extract, coffee (e.g., coffee beans), Sphingomonas elodea, Sphingomonas elodea extract, soy, carrot, sugar cane, Macrocystis pyrifera, Ulvaceae (Ulva intestinalis), olive, beet, baobob, lupin galactan, Karaya Gum, Yeast mannan extract, Orange Fiber, Tragacanth Gum, Tomato peels, Pea Fiber, Xanthan Gum, Spent Coffee Grounds, Gellan Gum, Soy Fiber, Carrot fiber, sugar cane, pacific kelp powder, Sea Lettuce Powder, Olive liquid waste, Beet pectin, Baobab powder, or any combination thereof.

In some aspects, the polysaccharides include one or more of amylose, amylopectin, beta glucan, pullulan, xyloglucan, arabinogalactan I, arabinogalactan II, rhamnogalacturonan I, rhamnogalacturonan II, polygalacturonic acid, polydextrose, galactan, arabinan, arabinoxylan, xylan (e.g., beechwood xylan), glycogen, mannan, glucomannan, curdlan, galactomannan, galactan, lichenan, inulin, fucoidan, xantham gum, gellen gum, cellulose or any combination thereof. In some aspects, the polysaccharides are from a plant or animal source. In some aspects, the polysaccharides are from a bacterial, yeast, or algal source. In some aspects, the polysaccharides are in the form of (optionally lyophilized) plant material. In some aspects, the plant material is locust bean gum, fenugreek seed, distiller's grain or spent distiller's grain or some fraction or extraction thereof. In some aspects, the method further comprises purifying one or more oligosaccharide from the mixture of oligosaccharides.

In some aspects, the polysaccharide material may be pre-treated with acids, bases, and/or oxidizing and reducing agents prior to reacting.

In some aspects, raw or natural sources and forms of polysaccharide-containing materials may be used. The poly saccharide-containing materials may be in a natural form, or may be permeabilized, ground, chopped, cavitated or otherwise divided or altered prior to contact with the reactants.

In some aspects, polysaccharides can be used which have varying degrees of branching, and having a variety of monosaccharide compositions, including natural and modified polysaccharides.

### Oligosaccharides and Oligosaccharide Compositions

The oligosaccharides and oligosaccharide compositions have suitable features, structural characteristics, and other various properties. In some aspects, the oligosaccharides or oligosaccharide compositions can have the features as described herein for any CLX composition, including CLX 101, CLX101C, CLX 102, CLX 103, CLX 105, CLX 107, CLX 108, CLX 109, CLX 110, CLX 111, CLX 112, CLX 113, CLX 114, CLX 115, CLX 115FC, CLX 115A, CLX115AL, CLX 116, CLX 117, CLX 118, CLX 119, CLX 121, CLX 122, CLX 122DS, CLX122DSF, CLX 123, CLX 124, CLX 125, CLX 126, CLX 127, CLX 128, CLX 129, CLX 130, CLX 131, CLX 132, CLX 133, or for any combination thereof. In some aspects, provided are compositions comprising a mixture of oligosaccharides (optionally purified) as generated using the disclosed methods, such as COG and/or FITDOG.

Disclosed herein are novel compositions comprising at least one synthetic oligosaccharide, which compositions, in some aspects, are useful as prebiotics, synbiotics, digestion aids, and food additives, among other various uses. Such compositions include those having CLX designations herein, as well as compositions having similar characteristics as such CLX compositions.

Disclosed herein is a synthetic composition comprising one or more oligosaccharides, wherein the one or more oligosaccharides collectively comprise arabinose, galactose, glucose, galacturonic acid, xylose, or rhamnose subunits, or any combination thereof.

Also disclosed herein is a synthetic composition comprising one or more oligosaccharides, wherein the one or more oligosaccharides collectively comprise arabinose, glucose, galactose, and xylose, or any combination thereof.

In some aspects, the oligosaccharides may be characterized (structure and/or activities/properties. In some aspects, high performance liquid chromatography-mass spectrometry (LC-MS) analysis of the product mixture shows a number of oligosaccharide structures ranging in size from a DP of 3 to as many as 20 or 30 or more (or from 3 to up to 200 for example), depending on the polysaccharide source and reaction conditions. The oligosaccharide structures and compositions generally will depend on the polysaccharide source(s).

In some aspects, the oligosaccharide composition can have different ranges of DP. In some aspects, the different ranges of DP have enhanced functions over other ranges of DP.

In some aspects, the oligosaccharides in an oligosaccharide composition can have the same or different ranges of DP. In some aspects, certain DPs have enhanced functions over other DPs. In some aspects, the DP is 2, 3, 4, 5, 6, 7, 8, 9, 10, 11, 12, 13, 14, 15, 16, 17, 18, 19, 20, 21, 22, 23, 24, 25, 26, 27, 28, 29, or 30. Each of the foregoing numbers can be preceded by the word “about,” “at least,” “at least about,” “less than,” or “less than about,” and any of the foregoing numbers can be used singly to describe a single point or an open-ended range, or can be used in combination to describe multiple single points or a close-ended range. For example, in some aspects, the DP is 2 to 6, 3 to 6, at least 2, less than 10, 2 to about 14, and the like.

In some aspects, each oligosaccharide with a particular DP or DP range can be present in an oligosaccharide composition at any suitable amount, based on the total weight of the composition. For example, in some aspects an oligosaccharide with a particular DP or DP range can be present in an amount (wt. %) of 1, 2, 3, 4, 5, 6, 7, 8, 9, 10, 12, 14, 16, 18, 20, 22, 24, 26, 28, 30, 35, 40, 45, 50, 55, 60, 65, 70, 75, 80, 85, 90, 95, 96, 97, 98, 99, or 100, based on the total weight of the oligosaccharide composition. For example, in some aspects, an oligosaccharide with a particular DP or DP range is present in an amount (wt. %) of 3 to 10, 5 to 14, less than 26, and the like, based on the total weight of the composition. These weight percents can apply to any of the DPs or DP ranges disclosed elsewhere herein.

In some aspects, each oligosaccharide with a particular DP or DP range can be present in an oligosaccharide composition at any suitable amount, based on the total weight of oligosaccharides having a DP of 2 to 10. For example, in some aspects an oligosaccharide with a particular DP or DP range can be present in an amount (wt. %) of 1, 2, 3, 4, 5, 6, 7, 8, 9, 10, 12, 14, 16, 18, 20, 22, 24, 26, 28, 30, 35, 40, 45, 50, 55, 60, 65, 70, 75, 80, 85, 90, 95, 96, 97, 98, 99, or 100, based on the total weight of oligosaccharides having a DP of 2 to 10. For example, in some aspects, an oligosaccharide with a particular DP or DP range is present in an amount (wt. %) of 20 to 50, 64 to 80, less than 30, at least 24, and the like, based on the total weight of the oligosaccharides having a DP of 2 to 10. These weight percents can apply to any of the DPs or DP ranges disclosed elsewhere herein.

In some aspects, the oligosaccharide composition can have different, enhanced, and unexpected properties when compared to its parent polysaccharide.

In some aspects, the oligosaccharide materials may be treated with suitable resin materials. Suitable resin materials may include anion-exchange, cation-exchange, decolorizing, chelation properties. For example, suitable resins may include, but are not limited to, Ionac NM-60, MBD-10 ULTRA, Thermax Tulsion MB, Cole-Parmer RR-1400, Amberlite MB20, DOWEX Monosphere MR-450. Two or more resins may be combined to create mixed-bed resins. The samples may be treated with carbon. The carbon may be activated carbon, charcoal, graphitized carbon, porous graphitized carbon, or any carbon-based material that is added with the goal of purification.

In some aspects carbohydrate active enzymes can be used to modify the resulting products by either adding or removing monomeric units to make a new product.

The resulting one or more (e.g., mixture of) oligosaccharides generated by the COG methods or any other method can have an average DP in the range of 2-200, e.g., 2-100 or 3-20 or 5-50, or any DP lower that the native polysaccharide, or any value in any subrange of the preceding exemplary ranges.

Some aspects of the present disclosure provide synthetic oligosaccharides comprising a backbone containing glucose monomers, wherein each glucose monomer is optionally bonded to a pendant xylose monomer, and wherein the total number of monomers in the synthetic oligosaccharide ranges from 3 to 30. Such synthetic oligosaccharides can be obtained, for example, by depolymerizing xyloglucan according to the methods described herein. Xyloglucan is known to contain a glucose backbone with single-unit xylose branches, where the xylose branches may be modified with a galactose endcap or an arabinose endcap. Tamarind xyloglucan, for example, contains a β1,4-linked glucose backbone with frequent single-unit branches of α1,6-linked xylose that can occasionally be further attached to a single β1,2-linked galactose endcap. In other sources of xyloglucan, arabinose can be α1,2 linked to the xylose residue. Xyloglucan from other sources may contain a single fucose residue α1,2 linked to the galactose.

In some aspects, the oligosaccharides comprise 2, 3, 4, 5, or 6 hexose subunits. In some aspects, the oligosaccharides comprise 1, 2, 3, or more pentose subunits. In some aspects, the oligosaccharides comprise a combination of hexose and pentose subunits, including any combination of the foregoing numbers, or any combination shown in any table herein. In some aspects, the oligosaccharides contain an equal number of hexose and pentose subunits. In some aspects the oligosaccharides contain fewer pentose residues than hexose subunits.

In some aspects, the glucose monomers in the backbone of the synthetic oligosaccharide are β1-4 linked glucose monomers. In some aspects, each pendant xylose monomer is bonded to a glucose monomer in the backbone by an α1-6 linkage.

In some aspects, the synthetic oligosaccharide further includes one galactose monomer bonded to one or more pendant xylose monomers. In some aspects, each galactose monomer is bonded to the pendant xylose monomer via a β1-2 linkage. In some aspects, the synthetic oligosaccharide further includes one fucose monomer bonded to one or more galactose monomers. In some aspects, each fucose monomer is bonded to the galactose monomer via an α1-2 linkage.

In some aspects, the synthetic oligosaccharide further includes one arabinose monomer bonded to one or more pendant xylose monomers. In some aspects, the arabinose monomer is bonded to the pendant xylose monomer via an α1-2 linkage.

In some aspects, the synthetic oligosaccharide contains 2 to 4 glucose monomer in the backbone, 1 to 2 pendant xylose monomers bonded to different glucose monomers in the backbone, and 0 to 2 galactose monomers bonded to different xylose monomers.

Some aspects of the present disclosure provide synthetic oligosaccharides having a backbone containing mannose monomers, wherein each mannose monomer is optionally bonded to a pendant galactose monomer, and wherein the total number of monomers in the synthetic oligosaccharide ranges from 3 to 30. Such synthetic oligosaccharides can be obtained, for example, by depolymerizing galactomannan according to the methods described herein. Galactomannan, produced by sources such as Aspergillus molds, contains a β1-4 mannose backbone with frequent α1-6 galactose branches containing a single unit.

Some aspects of the present disclosure provide synthetic oligosaccharides containing mannose monomers and glucose monomers, wherein the total number of monomers in the synthetic oligosaccharide ranges from 3 to 30. Such synthetic oligosaccharides can be obtained, for example, by depolymerizing glucomannan according to the methods described herein. Glucomannan is a polysaccharide largely known to be found in konjac root. The polymer contains β1-4-linked glucose and mannose residues that are thought to be randomly distributed in a non-reoccurring pattern.

Some aspects of the present disclosure provide synthetic oligosaccharides having a backbone containing arabinose monomers, wherein each arabinose monomer is optionally bonded to a pendant arabinose monomer, and wherein the total number of monomers in the synthetic oligosaccharide ranges from 3 to 30. Such synthetic oligosaccharides can be obtained, for example, by depolymerizing arabinan according to the methods described herein. Arabinans exist as sidechains on the pectin polysaccharide rhamnogalacturonan I and also in the cell walls of some mycobacteria. Arabinan contains an al-5 arabinose backbone with short α1-3 arabinose branches.

In some aspects of the present disclosure provide synthetic oligosaccharides derived from β-Glucans found in cereals (e.g., rice, wheat, oat, bran, barley, and malt), for example, consist of a β1-4 linked glucose backbone with single β1-3 glucose residues dispersed between every 2-3 β1-4 linked glucose residues. In some aspects of the present disclosure provide synthetic oligosaccharides derived from lichenan is a polysaccharide found in lichen, having a structure is similar to β3-glucan where the linkages consist of β1-4 and β1-3 glucose residues. However, unlike β3-glucan, lichenan has much more frequent β1-3 linkages. In some aspects, β3-glucan-resembling oligosaccharides can be derived from spent distillers' grain, or other corn products. In some aspects, β-glucan-resembling oligosaccharides can be derived from oat and oat agricultural waste products. In some aspects, β3-glucan-resembling oligosaccharides can be derived from spent brewers' grain, or other malt products.

Some aspects of the present disclosure provide synthetic oligosaccharides having a backbone containing xylose monomers, wherein each xylose monomer is optionally bonded to a pendant arabinose monomer or a pendant gluronic acid (e.g., a 4-O methylated GlcA), and wherein the total number of monomers in the synthetic oligosaccharide ranges from 3 to 30. Such synthetic oligosaccharides can be obtained, for example, by depolymerizing xylan and/or arabinoxylan according to the methods described herein. Xylan is a polysaccharide commonly found in the secondary cell walls of dicots and in the cell walls of most grasses. The structure contains a β1-4 xylose backbone and often times contains α1-2 glucuronic acid branches, which may contain a single methyl group. In some embodiments, beechwood xylan can be used, which is known to contain large amounts of 4-O-methyl-glucuronic acid branches. Arabinoxylan is a polysaccharide commonly found in cereals grains that contains a β1-4 xylose backbone with α1-2 and α1-3 arabinose branches. Some aspects of the present disclosure provide synthetic arabinoxylan-resembling oligosaccharides. Some aspects of the present disclosure provide synthetic arabinoxylan-resembling oligosaccharides from spent distillers' grain, corn fiber, or other corn-based streams. Some aspects of the present disclosure provide synthetic arabinoxylan-resembling oligosaccharides from spent distillers' grain, corn fiber, or other corn-based streams. Some aspects of the present disclosure provide synthetic arabinoxylan-resembling oligosaccharides from spent brewers' grain or other cereal-based streams.

In some aspects, synthetic oligosaccharides can be also be obtained by depolymerizing homopolymer polysaccharides according to the methods described herein. As used herein, the term “homopolymer polysaccharide” refers to a polysaccharide containing repeating monosaccharide subunits of the same kind, linked together by the same type of glycosidic bond including, but not limited to, a combination of 31-3 bonds, β1-4 bonds, β1-6 bonds, α1-3 bonds, al-4 bonds, and α1-6 bonds. Examples of homo polymers include, but are not limited to, curdlan, galactan, and mannan. Homopolymers include, but are not limited to, curdlan (a linear polymer of β1-3 linked glucose found as an exopolysaccharide of Agrobacterium), galactan (a linear polymer of β1-4 linked galactose that has been isolated in the form of arabinogalactan before subsequent arabinofuranosidase treatment to remove the arabinose units), and mannan (a linear polymer of β1-3 linked glucose found as an exopolysaccharide of Agrobacterium and also some nuts).

Also provided are mixtures containing two or more different synthetic oligosaccharides as described herein. Unpurified or semi-purified depolymerization products may be used for preparation of oligosaccharide mixtures or, alternatively, oligosaccharides can be purified to produce specially formulated pools. The synthetic oligosaccharides in the mixtures may be obtained, for example, by depolymerizing one or more polysaccharides. In some aspects, the amount of at least one of the synthetic oligosaccharides in the mixture is at least 1%, based on the total amount of oligosaccharides in the mixture. The synthetic oligosaccharide may be present, for example, in an amount ranging from about 1% to about 99%, or from about 5% to about 95%, or from about 10% to about 90%, or from about 20% to about 80%, or from about 30% to about 70%. The synthetic oligosaccharide may be present, for example, in an amount ranging from about 1% to about 10%, or from about 10% to about 20%, or from about 20% to about 30%, or from about 30% to about 40%, or from about 40% to about 50%, or from about 50% to about 60%, or from about 60% to about 70%, or from about 70% to about 80%, or from about 80% to about 90%, or from about 90% to about 99%. The percentage may be a mol %, based on the total number of moles of oligosaccharides in the mixture, or a weight %, based on the total weight of oligosaccharides in the mixture. In some aspects, the amount of at least one of synthetic oligosaccharides is at least 5 mol %.

### Uses for Oligosaccharides and Oligosaccharide Compositions

The oligosaccharides and oligosaccharide compositions disclosed herein have a variety of beneficial uses. Although several uses are disclosed in this section, there are other uses disclosed elsewhere herein in other sections, such as using the oligosaccharides, oligosaccharide compositions, or formulations thereof, for modulating microbiota and/or their metabolic outputs, or as therapeutics for health applications.

In some aspects, the oligosaccharides or oligosaccharide compositions disclosed herein can be combined with other ingredients to produce pharmaceutical compositions, foodstuffs, and supplements including infant formula, geriatric supplements, drinks, nutritional supplements, baking flours, and snack foods.

In some aspects, the described method will produce oligosaccharides for analysis and for bioactive foods that are prebiotic, anticancer, antipathogenic, or have other functions (to enhance biofuel production, the extractability of other compounds, etc.).

In some aspects, the oligosaccharides are bioactive oligosaccharides (e.g., bioactive oligosaccharides consumed by bacteria beneficial to the human gut). In some aspects the oligosaccharides are consumed by bacteria beneficial to the vaginal microbiome, beneficial to the respiratory tract, or beneficial to the skin. In some aspects the oligosaccharides are consumed by bacteria beneficial to the soil microbiome. In some aspects, the bioactive oligosaccharides function as a pathogen block. In some aspects the oligosaccharides are used as starting material for biofuel production. In some aspects the oligosaccharides can be used to modulate microbial metabolite output.

In some aspects, the one or more oligosaccharides, and/or the oligosaccharide compositions, are selective carbon substrates to stimulate growth of the microbiota of soils. In some aspects, the oligosaccharides are added to soil following a fumigation or sterilization protocols on the soil. Accessible organic carbon can drive the soil ecology in a pathogenic direction if uncontrolled. By providing specific oligosaccharides that selectively stimulate growth of (or provide a growth advantage to) beneficial soil microbiota, soil pathogen populations in the soil can be reduced. In some aspects, a combination of one or more oligosaccharide prepared as described herein can be added to soil with one or more microbe (e.g., beneficial soil microbes) to achieve a desired microbial complement or balance in the soil, or to reduce or eliminate pathogens or undesirable microbes. In some aspects, the oligosaccharides can selectively promote the growth and colonization of bacteria that can remediate soils by metabolizing contaminants or pollutants (e.g., chemicals, heavy metals, etc.) in soils. In some aspects, bacteria can be designed, through recombinant methods, to consume specific oligosaccharide structures. In some aspects, the oligosaccharides can selectively promote the growth of bacteria that, naturally or recombinantly, can produce insecticidal compounds. In some aspects, the oligosaccharides can selectively promote the growth of bacteria that produce, naturally or recombinantly, herbicidal compounds.

In some aspects, the oligosaccharides can be formulated into products for oral hygiene. In some aspects oral hygiene products can be tooth paste, mouth wash, chewing gum, mints, candies, lozenges, and floss.

In some aspects, the oligosaccharides or oligosaccharide compositions prepared herein, for example by the COG or FITDOG methods, can produce soluble fiber products. Soluble fiber products can be useful for a number of uses, including but not limited to medical products and devices, food products (i.e. thickeners, nutritional amendments, flavor agents and/or flavor modifiers), soil amendments (to engineer, balance or enrich specific beneficial soil microbiome constituents), and in fiber products (e.g., novel textiles, ropes, biodegradable packaging, etc.). In some aspects, for example, the insoluble fiber is cotton, which may be treated, or partially treated using the COG or FITDOG methods described herein to achieve one or more desired characteristics (e.g., softness, strength, resiliency, absorbency, etc.). In some aspects, COG or FITDOG methods described herein can modify insoluble fiber to make it soluble.

In some aspects, one or more (e.g., mixture of) oligosaccharides generated by the COG methods or by any other method can have a variety of uses. In some aspects, the one or more oligosaccharides can be used as a prebiotic to selectively stimulate growth of one or more probiotic bacteria. In some aspects, the oligosaccharide compositions can be administered as a prebiotic formulation (i.e., without bacteria) or as a probiotic formulation (i.e., with one or more desirable bacteria such as bifidobacteria as described herein). In general, any food or beverage that can be consumed by humans or animals, or otherwise suitably administered, may be used to make formulations containing the prebiotic and probiotic oligosaccharide containing compositions.

In some aspects, the oligosaccharide compositions can be used as bulking-agents. In some aspects, the oligosaccharide compositions can be used as bulking-agents in reduced sugar food applications. In some aspects these oligosaccharides can be used as bulking-agents that do not affect flavor, odor, rheological, and textural properties. In some aspects, the oligosaccharides and oligosaccharide compositions are employed in foods, beverages, or medicinal formulations in a manner that affects the rheological and/or textural properties of the foods, beverages, or medicinal formulations.

In some aspects, the oligosaccharide compositions are administered to a subject for promotion of resistance to bacterial or yeast infections, e.g., Candidiasis or diseases induced by sulfate reducing bacteria.

In some aspects, the synthetic oligosaccharides and compositions described herein are useful as synbiotics, prebiotics, immune modulators, digestion aids, food additives, pharmaceutical excipients, or analytical standards. The synthetic oligosaccharides can be combined with other ingredients to produce foodstuffs and supplements including infant formula, geriatric supplements, baking flours, or snack foods. The synthetic oligosaccharides can be combined with beneficial bacteria to form synbiotics. The synthetic oligosaccharides can also be used as pharmaceutical products.

In some aspects, the synthetic oligosaccharides can be used as for growth or maintenance of specific microorganism in humans, other mammals, or in the rhizosphere of plants. The synthetic oligosaccharides may contain specific glycosidic linkages not able to be digested by the particular host (e.g., a person, livestock animal, or companion animal) but able to be metabolized by specific groups of commensal microorganism or probiotics. As such, the synthetic oligosaccharides can function as a carrier to transport exogenous microorganisms (e.g., probiotic) to a specific niche, or as a nutritional source for microorganisms already present in the host.

### Formulations and Administration

The oligosaccharides and oligosaccharide compositions disclosed herein can be formulated into a variety of formulations or compositions, and such formulations or compositions can be administered to an organism (e.g., a patient, mammal, human, etc.) in a variety of ways. In some aspects, the oligosaccharides and oligosaccharide compositions can be formulated, for example, into a nutritional composition, pharmaceutical composition, or other composition or formulation.

Administration of and administering a compound or composition should be understood to mean providing a compound or salt thereof or a pharmaceutical composition comprising a compound, in which the compound is a synthetic oligosaccharide or a composition comprising a synthetic oligosaccharide, optionally including additional active ingredients, such as antibiotics, probiotics, and/or prebiotics. The compound or composition can be administered by another person to the patient (e.g., orally, intravenously, and/or topically) or it can be self-administered by the subject (e.g., orally, such as via tablets or capsules, and/or topically via a cream, ointment, or gel). The term “subject,” “patient,” and similar terms generally refer to mammals (for example, humans and veterinary animals such as dogs, cats, pigs, horses, sheep, and cattle). Administration of one or more of the compositions disclosed herein in combination with other compounds, such as excipients, colorants, fillers, binders, diluents, buffering agents, moistening agents, preservatives, flavoring agents, dyes, disintegrating agents, pharmaceutically compatible carriers, or any combination thereof, is contemplated.

The pharmaceutical compositions can be administered as oral, sublingual, transdermal, subcutaneous, topical, absorption through epithelial or mucocutaneous linings, intravenous, intranasal, intraarterial, intramuscular, intratumoral, peritumoral, interperitoneal, intrathecal, rectal, vaginal, or aerosol formulations. In some aspects, the pharmaceutical composition is administered orally (e.g., enteral) or intravenously (e.g., parenteral).

In some aspects, the oligosaccharides can be formulated into products for oral hygiene. In some aspects oral hygiene products can be tooth paste, mouth wash, chewing gum, mints, candies, lozenges, and floss. In some aspects, the oligosaccharides are formulated at approximately 10 mg/application. In some aspects, the oligosaccharides can be formulated at approximately 100 mg/application. In some aspects, the oligosaccharides can be formulated at approximately 200 mg or more/application.

In some aspects, one or more oligosaccharide compositions as described herein can be used to supplement a beverage. Examples of such beverages include, without limitation, infant formula, follow-on formula, toddler's beverage, milk, fermented milk, fruit juice, fruit-based drinks, and sports drinks. Many infant and toddler formulas are known in the art and are commercially available, including, for example, Carnation Good Start™ (Nestle Nutrition Division; Glendale, Calif.) and Nutrish AB™ produced by Mayfield Dairy Farms (Athens, Tenn.). Other examples of infant or baby formula include those disclosed in U.S. Pat. No. 5,902,617, hereby incorporated by reference in its entirety for all purposes. Other beneficial formulations of the compositions include the supplementation of animal milks, such as cow's milk.

Alternatively, the formulations (e.g., oligosaccharide compositions) can be formulated into pills or tablets or encapsulated in capsules, such as gelatin capsules. Tablet forms can optionally include, for example, one or more of lactose, sucrose, mannitol, sorbitol, calcium phosphates, corn starch, potato starch, alginic acid, microcrystalline cellulose, acacia, gelatin, colloidal silicon dioxide, talc, magnesium stearate, stearic acid, and other excipients, colorants, fillers, binders, diluents, buffering agents, moistening agents, preservatives, flavoring agents, dyes, disintegrating agents, and pharmaceutically compatible carriers. Lozenge or candy forms can comprise the compositions in a flavor, e.g., sucrose, as well as pastilles comprising the compositions in an inert base, such as gelatin and glycerin or sucrose and acacia emulsions, gels, and the like containing, in addition to the active ingredient, carriers known in the art. The prebiotic or probiotic oligosaccharide containing formulations may also contain conventional food supplement fillers and extenders such as, for example, rice flour. The products may also be used to help the absorption of other nutrients and minerals.

In some aspects, the formulations (e.g., oligosaccharide compositions) will comprise or further comprise a non-human protein, non-human lipid, non-human carbohydrate, or other non-human component. For example, in some aspects, the compositions may comprise a bovine (or other non-human) milk protein, a soy protein, a rice protein, beta-lactoglobulin, whey, soybean oil or starch. In some aspects, the oligosaccharides are combined with polysaccharides. In some aspects, the oligosaccharides are combined with their parent polysaccharide.

In some aspects, formulations (e.g., oligosaccharide compositions) as described herein are in the form of a nutritional composition. The nutritional composition can be a food, a beverage, a rehydration solution, a medical food or food for special medical purposes, a nutritional supplement, and the like. The nutritional composition can contain sources of protein, lipids and/or digestible carbohydrates and can be in solid, powdered, or liquid forms. The synthetic composition can be designed to be the sole source of nutrition, or as a food or nutritional supplement which forms part of the diet.

Suitable protein sources include milk proteins, soy protein, rice protein, pea protein and oat protein, or mixtures thereof. Milk proteins can be in the form of milk protein concentrates, milk protein isolates, whey protein or casein, or mixtures of both. The protein can be whole protein or hydrolyzed protein, either partially hydrolyzed or extensively hydrolyzed. Hydrolyzed protein offers the advantage of easier digestion which can be important for humans with inflamed or compromised GI tracts. The protein can also be provided in the form of free amino acids. The protein can comprise about 5% to about 30% of the energy of the nutritional composition, normally about 10% to 20%.

The protein source can be a source of glutamine, threonine, cysteine, serine, proline, or a combination of these amino acids. The glutamine source can be a glutamine dipeptide and/or a glutamine enriched protein. Glutamine can be included due to the use of glutamine by enterocytes as an energy source. Threonine, serine, and proline are important amino acids for the production of mucin. Mucin coats the gastrointestinal tract and can improve intestinal barrier function and mucosal healing. Cysteine is a major precursor of glutathione, which is key for the antioxidant defenses of the body.

Suitable digestible carbohydrates include maltodextrin, hydrolyzed or modified starch or corn starch, glucose polymers, corn syrup, corn syrup solids, high fructose corn syrup, rice-derived carbohydrates, pea-derived carbohydrates, potato-derived carbohydrates, tapioca, sucrose, glucose, fructose, sucrose, lactose, honey, sugar alcohols (e.g., maltitol, erythritol, sorbitol), or mixtures thereof. Preferably the composition is reduced in or free from added lactose or other FODMAP carbohydrates. Generally digestible carbohydrates provide about 35% to about 55% of the energy of the nutritional composition. A particularly suitable digestible carbohydrate is a low dextrose equivalent (DE) maltodextrin.

Suitable lipids include medium chain triglycerides (MCT) and long chain triglycerides (LCT). Preferably the lipid is a mixture of MCTs and LCTs. For example, MCTs can comprise about 30% to about 70% by weight of the lipids, more specifically about 50% to about 60% by weight. MCTs offer the advantage of easier digestion which can be important for humans with inflamed or compromised GI tracts. Generally, the lipids provide about 35% to about 50% of the energy of the nutritional composition. The lipids can contain essential fatty acids (omega-3 and omega-6 fatty acids). Preferably these polyunsaturated fatty acids provide less than about 30% of total energy of the lipid source.

Suitable sources of long chain triglycerides are rapeseed oil, sunflower seed oil, palm oil, soy oil, milk fat, corn oil, high oleic oils, and soy lecithin. Fractionated coconut oils are a suitable source of medium chain triglycerides. The lipid profile of the nutritional composition is preferably designed to have a polyunsaturated fatty acid omega-6 (n-6) to omega-3 (n-3) ratio of about 4:1 to about 10:1. For example, the n-6 to n-3 fatty acid ratio can be about 6:1 to about 9:1.

The formulation (e.g., nutritional composition) may also include vitamins and minerals. If the nutritional composition is intended to be a sole source of nutrition, it preferably includes a complete vitamin and mineral profile. Examples of vitamins include vitamins A, B-complex (such as B1, B2, B6 and B12), C, D, E and K, niacin, and acid vitamins such as pantothenic acid, folic acid and biotin. Examples of minerals include calcium, iron, zinc, magnesium, iodine, copper, phosphorus, manganese, potassium, chromium, molybdenum, selenium, nickel, tin, silicon, vanadium, and boron.

The nutritional composition can also include a carotenoid such as lutein, lycopene, zeaxanthin, and beta-carotene. The total amount of carotenoid included can vary from about 0.001 g/ml to about 10 μg/ml. Lutein can be included in an amount of from about 0.001 μg/ml to about 10 g/ml, preferably from about 0.044 μg/ml to about 5 μg/ml of lutein. Lycopene can be included in an amount from about 0.001 μg/ml to about 10 μg/ml, preferably about 0.0185 μg/ml to about 5 μg/ml of lycopene. Beta-carotene can comprise from about 0.001 μg/ml to about 10 mg/ml, for example about 0.034 μg/ml to about 5 μg/ml of beta-carotene.

The nutritional composition preferably also contains reduced concentrations of sodium; for example, from about 300 mg/l to about 400 mg/l. The remaining electrolytes can be present in concentrations set to meet needs without providing an undue renal solute burden on kidney function. For example, potassium is preferably present in a range of about 1180 to about 1300 mg/l; and chloride is preferably present in a range of about 680 to about 800 mg/l.

The nutritional composition can also contain various other conventional ingredients such as preservatives, emulsifying agents, thickening agents, buffers, fiber and prebiotics (e.g. fructooligosaccharides, galactooligosaccharides), probiotics (e.g. B. animalis subsp. lactis BB-12, B. lactis HN019, B. lactis Bi07, B. infantis ATCC 15697, L. rhamnosus GG, L. rhamnosus HNOOl, L. acidophilus LA-5, L. acidophilus NCFM, L. fermentum CECT5716, B. longum BB536, B. longum AH1205, B. longum AH1206, B. breve M-16V, L. reuteri ATCC 55730, L. reuteri ATCC PTA-6485, L. reuteri DSM 17938), antioxidant/anti-inflammatory compounds including tocopherols, carotenoids, ascorbate/vitamin C, ascorbyl palmitate, polyphenols, glutathione, and superoxide dismutase (melon), other bioactive factors (e.g. growth hormones, cytokines, TFG-3), colorants, flavors, and stabilizers, lubricants, and so forth.

The nutritional composition can be formulated as a soluble powder, a liquid concentrate, or a ready-to-use formulation. The composition can be fed to a human in need via a nasogastric tube or orally. Various flavors, fibers and other additives can also be present.

The nutritional compositions can be prepared by any commonly used manufacturing techniques for preparing nutritional compositions in solid or liquid form. For example, the composition can be prepared by combining various feed solutions. A protein-in-fat feed solution can be prepared by heating and mixing the lipid source and then adding an emulsifier (e.g., lecithin), fat soluble vitamins, and at least a portion of the protein source while heating and stirring. A carbohydrate feed solution is then prepared by adding minerals, trace, and ultra-trace minerals, thickening or suspending agents to water while heating and stirring. The resulting solution is held for 10 minutes with continued heat and agitation before adding carbohydrates (e.g., the oligosaccharides described herein and digestible carbohydrate sources). The resulting feed solutions are then blended while heating and agitating and the pH adjusted to 6.6-7.0, after which the composition is subjected to high-temperature short-time processing during which the composition is heat treated, emulsified and homogenized, and then allowed to cool. Water soluble vitamins and ascorbic acid are added, the pH is adjusted to the desired range if necessary, flavors are added, and water is added to achieve the desired total solid level.

For a liquid product, the resulting solution can then be aseptically packed to form an aseptically packaged nutritional composition. In this form, the nutritional composition can be in ready-to-feed or concentrated liquid form. Alternatively, the composition can be spray-dried and processed and packaged as a reconstitutable powder.

When the nutritional product is a ready-to-feed nutritional liquid, it may be preferred that the total concentration of the oligosaccharides or oligosaccharide compositions in the liquid, by weight of the liquid, is from about 0.1% to about 1.5%, including from about 0.2% to about 1.0%, for example from about 0.3% to about 0.7%. When the nutritional product is a concentrated nutritional liquid, it may be preferred that the total concentration of oligosaccharide compositions in the liquid, by weight of the liquid, is from about 0.2% to about 3.0%, including from about 0.4% to about 2.0%, for example from about 0.6% to about 1.5%.

The nutritional composition can also be in a unit dosage form or as a pharmaceutical composition. The unit dosage form can contain an acceptable food-grade carrier, e.g., phosphate buffered saline solution, mixtures of ethanol in water, water and emulsions such as an oil/water or water/oil emulsion, as well as various wetting agents or excipients. The unit dosage form can also contain other materials that do not produce an adverse, allergic, or otherwise unwanted reaction when administered to a subject. The carriers and other materials can include solvents, dispersants, coatings, absorption promoting agents, controlled release agents, and one or more inert excipients, such as starches, granulating agents, microcrystalline cellulose, diluents, lubricants, binders, and disintegrating agents. Preferably carriers and other materials are low in FODMAPs or contain no FODMAPs.

The unit dosage form or pharmaceutical composition can be administered orally, e.g., as a tablet, capsule, or pellet containing a predetermined amount of the mixture, or as a powder or granules containing a predetermined concentration of the mixture or a gel, paste, solution, suspension, emulsion, syrup, bolus, electuary, or slurry, in an aqueous or non-aqueous liquid, containing a predetermined concentration of the mixture. An orally administered composition can include one or more binders, lubricants, inert diluents, flavoring agents, and humectants. An orally administered composition such as a tablet can optionally be coated and can be formulated to provide sustained, delayed, or controlled release of the oligosaccharide compositions.

The oligosaccharide composition may take any form (e.g., as a formulation) which is suitable for delivery the oligosaccharide into the gastrointestinal tract (including the stomach and rectum) of the subject. Suitable forms include enterally administered nutritional compositions, orally administered unit dosage forms, buccally administered unit dosage forms, and rectally administered unit dosage forms. The enterally administered nutritional compositions may be suitable for administration through a nasogastric tube, through a jejunum tube, orally, and the like. The enterally administered nutritional composition may be suitable for administration through a nasogastric tube, through a jejunum tube, orally, and the like. In embodiments, the enterally administered nutritional composition can include other components of nutritional value and can be formulated as a soluble powder, a liquid concentrate, a ready-to-use formulation, a food, a snack, and the like. In embodiments, the orally administered unit dosage form can be a tablet, a capsule, a pellet, a powder, a gel, a paste, a solution, a suspension, an emulsion, a syrup, a liquid, and the like. The orally administered unit dosage form can be coated and/or formulated to provide sustained, delayed or controlled release of the oligosaccharide, and can contain other active components. The orally administered unit dosage form can be formulated for pharmaceutical use, dietary supplement use or nutritional use. The buccally administered unit dosage form is conveniently in the form of a tablet, pellet, wafer, film, patch, spray, drop or gel suitable for delivery into the buccal cavity, include for sublingual delivery. The rectally administered unit dosage form is conveniently a suppository, a capsule, a tablet, an enemas, a gel, a foam, a cream and the like. The buccally and rectally administered dosage forms can include other active components.

The unit dosage form or pharmaceutical composition can also be administered by rectal suppository, aerosol tube, naso-gastric tube or direct infusion into the GI tract or stomach.

The unit dosage form or pharmaceutical composition can also include agents such as antibiotics, probiotics, analgesics, and anti-inflammatory agents.

The proper dosage of the unit dosage form, pharmaceutical composition, and the nutritional composition can be determined in a conventional manner, based upon factors such as the subject's condition, immune status, body weight and age. In general, the dosage of the unit dosage form, pharmaceutical composition, and the nutritional composition is such that the amount of oligosaccharide composition or oligosaccharide delivered is in the range from about 0.5 g to about 15 g per day, in certain aspects from about 1 g to about 10 g per day, for example about 2 g to about 7.5 g per day. Appropriate dose regimes can be determined by methods known to those skilled in the art. The amount of the oligosaccharide or oligosaccharide composition in this paragraph are based on total weight of oligosaccharides on a dry basis.

In some aspects, the oligosaccharides or oligosaccharide compositions may be in the form of an enterally administered composition, a topically administered composition, an intra-vaginally administered composition, or disposable absorbent article such as a diaper, a pant, an adult incontinence product, an absorbent insert for a diaper or pant, a wipe or a feminine hygiene product, such as a sanitary napkin, a tampon and a panty liner.

In some aspects, enterally administered composition contains an amount of 0.5 g to 15 g of an oligosaccharide or oligosaccharide composition, more preferably 1 g to 10 g. For example, the enterally administered composition may contain 2 g to 7.5 g of an oligosaccharide or oligosaccharide composition. The topically administered oligosaccharide or oligosaccharide composition and the intra-vaginally administered oligosaccharide or oligosaccharide composition preferably contain an amount of 0.1 g to 10 g of the oligosaccharide or oligosaccharide composition, more preferably 0.2 g to 7.5 g. For example, the topically or intra-vaginally administered oligosaccharide or oligosaccharide composition may contain 0.5 g to 5 g of the oligosaccharide or oligosaccharide composition. When in the form of a disposable absorbent article, at least a portion of the article may be coated or impregnated with an oligosaccharide or oligosaccharide composition in an amount of 0.2 g to 200 g per square meter, preferably between 5.0 g and 100 g per square meter, more preferably between 8.0 g and 50 g per square meter. In the case of a female requiring improvement in urogenital health or treatment, the female may be administered a higher dose initially followed by a lower dose. The higher dose is preferably administered for up to 14 days, for example up to 7 days. The lower dose may be administered over an extended period of time. In the case of a female requiring management to reduce the risk of bacterial vaginosis, recurrence of bacterial vaginosis, urinary tract infection or recurrence of urinary tract infection, the female may be administered a lower maintenance dose over an extended period of time. The amount of the oligosaccharide or oligosaccharide composition in this paragraph are based on total weight of oligosaccharides on a dry basis.

In some aspects, the oligosaccharide is administered for at least 14 days, more preferably at least 21 days. For example, the oligosaccharide may be administered for at least 28 days.

In some aspects, the oligosaccharide is administered an amount of 0.5 g to 15 g per day; more preferably 1 g to 10 g per day. For example, the oligosaccharide may be administered in an amount of 2 g to 7.5 g per day. The amount of the oligosaccharide or oligosaccharide composition in this paragraph are based on total weight of oligosaccharides on a dry basis.

In some aspects, the patient may be administered a higher dose initially followed by a lower dose. The higher dose can be about 2 g to about 15 g, or about 3 g to about 10 g per day (for example about 4 g to about 7.5 g per day) and the lower dose can be about 2 g to about 7.5 g per day (for example about 2 g to about 5 g per day). The higher dose can be administered for up to 14 days; for example up to 7 days. The lower dose can be administered chronically; for example at least 28 days. The amount of the oligosaccharide or oligosaccharide composition in this paragraph are based on total weight of oligosaccharides on a dry basis.

In some aspects, the patient may be administered a bifidobacteria or lactobacillus in addition to the one or more oligosaccharides. The bifidobacteria may be, for example, Bifidobacterium longum, Bifidobacterium infantis and/or Bifidobacterium bifidum. The lactobacillus may be, for example, Lactobacillus rhamnosus

The amount of oligosaccharide required to be administered for treating or reducing the risk of occurrence of chronic gastrointestinal conditions associated with an impaired intestinal barrier function, treating or reducing the risk of occurrence of a chronic metabolic condition, treating or reducing the risk of occurrence of a chronic kidney condition, treating or reducing the risk of occurrence of an atopic allergy, and/or treating or reducing the risk of occurrence of a chronic medical condition associated with dysfunction in gut brain interactions, will vary depending upon factors such as the risk and severity of the underlying condition, any other medical conditions or diseases, age, the form of the composition, and other medications being administered. Further the amount may vary depending upon whether the oligosaccharide is being used to deliver a direct effect (when the dose may be higher) or whether the oligosaccharides are being used as a secondary prevention/maintenance (when the dose may be lower). However, the required amount can be readily set by a medical practitioner considering, for example, the factors in this paragraph and elsewhere herein, and would generally be in the range from about 0.5 g to about 15 g per day, in certain embodiments from about 1 g to about 10 g per day, for example from about 2 g to about 7.5 g per day. An appropriate dose can be determined based on several factors, including, for example, body weight and/or condition, the severity of the underlying condition being treated or prevented, other ailments and/or diseases, the incidence and/or severity of side effects and the manner of administration. Appropriate dose ranges may be determined by methods known to those skilled in the art. For example, for a subject to be treated for a condition, disease, disorder, or indication, the subject may be administered a higher dose initially. For example, the higher dosing can be, for example, 2 g to 15 g, 3 g to 15 g per day, or 4 g to 7.5 g per day. The higher dosing phase may be followed by a maintenance phase, or the subject may be started on the maintenance phase, if desired, where a lower dose is administered. During a maintenance phase, the dosing can be reduced (for example, 1 g to 10 g per day, preferably 2 g to 7.5 g per day, more preferably about 2 g to about 5 g per day)). The lower dose may be in the range from about 0.5 g to about 10 g per day, in certain embodiments from about 1 g to about 7.5 g per day, for example about 2 g to about 5 g per day. For a subject in need of preventative or prophylactic treatment, the patient may be administered a substantially constant dose over the intervention period. The preventative dose may be administered for more than 14 days, for example up to 21 days, but may also be administered over an extended period of time such as more than 28 days. The preventative dose may be in the range from about 0.5 g to about 10 g per day, in certain embodiments from about 1 g to about 7.5 g per day, for example about 2 g to about 5 g per day. The administration may be once a day or may involve multiple administrations per day, preferably once a day. The amount of the oligosaccharide or oligosaccharide composition in this paragraph are based on total weight of oligosaccharides on a dry basis.

In embodiments where the enteral composition contains further active compounds, the dosage of the enteral composition may such that the amount of oligosaccharide composition delivered is in the range from about 0.1 g to about 15 g per day, in certain embodiments from about 0.5 g to about 10 g per day, for example about 1 g to about 7.5 g per day. The administration may be once a day or may involve multiple administrations per day, preferably once a day. The amount of the oligosaccharide or oligosaccharide composition in this paragraph are based on total weight of oligosaccharides on a dry basis.

In some aspects, the oligosaccharide or oligosaccharide compositions described herein can be used to generate a prebiotic for food supplementation. In some aspects, the oligosaccharide or oligosaccharide composition can be used to modulate appetite control and/or control of energy (caloric) intake in subject in need thereof (e.g., children, or other subjects, with excess weight and obesity).

In some aspects, the oligosaccharide or oligosaccharide compositions can be administered as a prebiotic formulation (i.e., without bacteria) or as a probiotic formulation (i.e., with one or more desirable bacteria such as Bifidobacteria or Lactobacillus as described elsewhere herein). In general, any food or beverage that can be consumed by humans or animals, or otherwise suitably administered or topically applied, may be used to make formulations containing the prebiotic and probiotic oligosaccharide containing compositions. Exemplary foods include those with a semi-liquid consistency to allow easy and uniform dispersal of the prebiotic and probiotic compositions described herein. However, other consistencies (e.g., powders, liquids, etc.) can also be used without limitation. Accordingly, such food items include, without limitation, dairy-based products such as cheese, cottage cheese, yogurt, and ice cream. Processed fruits and vegetables, including those targeted for infants/toddlers, such as apple sauce or strained peas and carrots, are also suitable for use in combination with the oligosaccharides of disclosed herein. Both infant cereals such as rice- or oat-based cereals and adult cereals such as Cream of Wheat™, etc., are also suitable for use in combination with the oligosaccharides. The oligosaccharide or oligosaccharide composition can also be used in medical foods, for example, such as Pedialyte™, Ensure™, etc. In addition to foods targeted for human consumption, animal feeds may also be supplemented with the prebiotic and probiotic oligosaccharide containing compositions.

In some aspects, the dosages of the formulations or compositions (e.g., prebiotic and probiotic oligosaccharide containing compositions) will vary depending upon the requirements of the individual, and/or will take into account factors such as age (infant versus adult), weight, and reasons for loss of beneficial gut bacteria (e.g., antibiotic therapy, chemotherapy, radiation therapy, disease, or age). The administration regimen, and amount administered to, or consumed by an individual, in the context of the present disclosure should preferably be sufficient to establish colonization of the gut with beneficial bacteria over time. The administration regimen and/or the size of the dose also will be determined by the existence, nature, and extent of any adverse side-effects that may accompany the administration of the provided prebiotic or probiotic oligosaccharide containing compositions. In some administration aspects, the dosage range will be effective as a food supplement and for reestablishing beneficial bacteria in the intestinal tract. In some administration aspects, the dosage of an oligosaccharide composition disclosed herein ranges from about 1 micrograms/L to about 25 grams/L of oligosaccharides. In some aspects, the dosage of an oligosaccharide composition is about 100 micrograms/L to about 15 grams/L of oligosaccharides. In some aspects, the dosage of an oligosaccharide composition is about 1-10 g/L, 5-15 g/L, 10-50 g/L, or as high as 200 g/L. In some aspects, the dosage is 50-70 g/day. In some aspects, the dosage is 10 g/day. In some aspects, the dosage is between 1 and 10 g/day. In some aspects, the dosage is over 100 g/day. In some aspects, the dosage is 0.25-3 g/day. Exemplary Bifidobacterium dosages include, but are not limited to, about 10⁴ to about 10¹² colony forming units (CFU) per dose. A further advantageous range is about 10⁶ to about 10¹⁰ CFU. Other bacterium can also be dosed at similar concentrations, but are not limited to, about 104 to about 10¹² colony forming units (CFU) per dose or about 10⁶ to about 10¹⁰ CFU. The amount of the oligosaccharide or oligosaccharide composition in this paragraph are based on total weight of oligosaccharides on a dry basis.

In some aspects, the disclosed formulations (e.g., prebiotic or probiotic oligosaccharide containing formulations) can be administered to any subject/individual in need thereof. In some aspects, the individual is an infant or toddler. For example, in some aspects, the individual is less than, e.g., 3 months, 6 months, 9 months, one year, two years or three years old. In some aspects, the individual is between 3-18 years old. In some aspects, the individual is an adult (e.g., 18 years or older). In some aspects, the individual is over 50, 55, 60, 65, 70, or 75 years old. In some aspects, the subject is a female (e.g., assigned female at birth or reassigned as female with or without surgery and with or without hormone treatment). In some aspects, the subject is a male (e.g., assigned male at birth or reassigned as male with or without surgery and/or hormone treatment). In some aspects, the subject is female or male. In some aspects, the individual is immuno-deficient (e.g., the individual has AIDS or is taking chemotherapy, immunotherapy, or radiation therapy).

In some aspects, the disclosed formulations can include probiotics, such as Bifidobacterium. Exemplary Bifidobacterium include, but are not limited to, Bifidobacterium longum subsp. infantis, B. longum subsp. longum, Bifidobacterium breve, Bifidobacterium adolescentis, B. pseudocatenulatum, or any combination thereof. The Bifidobacterium used will depend in part on the target consumer. Exemplary Lactobacillus that can be included in the oligosaccharide compositions disclosed herein include, but are not limited to, Lactobacillus crispatus, Lactobacillus jensenii, Lactobacillus gasseri, Lactobacillus iners, Lactobacillus vaginalis, and any combination thereof.

In some aspects, other components may be included in formulations. For example, it will be appreciated that it may be advantageous for some applications to include additional components in the compositions and formulations described herein. Such additional components may include, but are not limited to, Bifidogenic factors, fructoligosaccharides such as RAFFINOSE (Rhone-Poulenc, Cranbury, New Jersey), inulin (Imperial Holly Corp., Sugar Land, Texas), and NUTRAFLORA (Golden Technologies, Westminister, Colorado), as well as lactose, xylooligosaccharides, soyoligosaccharides, lactulose/lactitol and galactooligosaccharides among others. In some applications, other beneficial bacteria, such as Lactobacillus, Rumminococcus, Akkermansia, Bacteroides, Faecalibacterium can be included in the compositions. The oligosaccharides and oligosaccharide compositions described herein can also be used to stimulate yeast.

In some aspects, the formulation comprising oligosaccharides or oligosaccharide compositions is in the form of a pharmaceutical composition. Pharmaceutical compositions herein comprise a named active ingredient (e.g., oligosaccharide or oligosaccharide composition) in an amount effective for achieving the desired biological activity for a given form of administration to a given patient and optionally contain a pharmaceutically acceptable carrier. Pharmaceutical compositions can include an amount (for example, a unit dosage) of one or more of the disclosed oligosaccharides or other active ingredient together with one or more non-toxic pharmaceutically acceptable additives, including carriers, diluents, and/or adjuvants, and optionally other biologically active ingredients. Such pharmaceutical compositions can be prepared by standard pharmaceutical formulation techniques such as those disclosed in Remington's Pharmaceutical Sciences, Mack Publishing Co., Easton, Pa. (19th Edition).

Pharmaceutically acceptable carriers are those carriers that are compatible with the other ingredients in the formulation and are biologically acceptable. Carriers can be solid or liquid. It is currently contemplated that preferred carrier are liquid carriers. Carriers can include one or more substances that can also act as solubilizers, suspending agents, fillers, glidants, compression aids, binders, tablet-disintegrating agents, or encapsulating materials. Liquid carriers can be used in preparing solutions, suspensions, emulsions, syrups and elixirs. The active ingredient can be dissolved or suspended in a pharmaceutically acceptable liquid carrier such as water (of appropriate purity, e.g., pyrogen-free, sterile, etc.), an organic solvent, a mixture of both, or a pharmaceutically acceptable oil or fat. The liquid carrier can contain other suitable pharmaceutical additives such as, for example, solubilizers, emulsifiers, buffers, preservatives, sweeteners, flavoring agents, suspending agents, thickening agents, colors, viscosity regulators, stabilizers or osmo-regulators. Compositions for oral administration can be in either liquid or solid form.

Suitable examples of liquid carriers for oral and parenteral administration include water of appropriate purity, aqueous solutions (particularly containing additives, e.g. cellulose derivatives, sodium carboxymethyl cellulose solution), alcohols (including monohydric alcohols and polyhydric alcohols e.g. glycols) and their derivatives, and oils. Sterile liquid carriers are used in sterile liquid form compositions for parenteral administration and can include water, saline, aqueous dextrose and related sugar solutions, an alcohol, such as ethanol, isopropanol, or hexadecyl alcohol, glycols, such as propylene glycol or polyethylene glycol, glycerol ketals, such as 2,2-dimethyl-1,3-dioxolane-4-methanol, ethers, such as poly(ethyleneglycol) 400, an oil, a fatty acid, a fatty acid ester or glyceride, or an acetylated fatty acid glyceride with or without the addition of a pharmaceutically acceptable surfactant, such as a soap or a detergent, suspending agent, such as pectin, carbomers, methylcellulose, hydroxypropylmethylcellulose, or carboxymethylcellulose, or emulsifying agents and other pharmaceutical adjuvants. The liquid carrier for pressurized compositions can be halogenated hydrocarbon or other pharmaceutically acceptable propellant. Liquid pharmaceutical compositions that are sterile solutions or suspensions can be administered by, for example, intramuscular, intraperitoneal or subcutaneous injection. Sterile solutions can also be administered intravenously. Compositions for oral administration can be in either liquid or solid form. The carrier can also be in the form of creams and ointments, pastes, and gels. The creams and ointments can be viscous liquid or semisolid emulsions of either the oil-in-water or water-in-oil type.

Oils, which can be used in parenteral formulations include petroleum, animal, vegetable, or synthetic oils. Specific examples of oils include peanut, soybean, sesame, cottonseed, corn, olive, petrolatum, and mineral. Suitable fatty acids for use in parenteral formulations include oleic acid, stearic acid, and isostearic acid. Ethyl oleate and isopropyl myristate are examples of suitable fatty acid esters. Suitable soaps for use in parenteral formulations include fatty alkali metal, ammonium, and triethanolamine salts, and suitable detergents include (a) cationic detergents such as, for example, dimethyl dialkyl ammonium halides, and alkyl pyridinium halides, (b) anionic detergents such as, for example, alkyl, aryl, and olefin sulfonates, alkyl, olefin, ether, and monoglyceride sulfates, and sulfosuccinates, (c) nonionic detergents such as, for example, fatty amine oxides, fatty acid alkanolamides, and polyoxyethylene-polypropylene copolymers, (d) amphoteric detergents such as, for example, alkyl-beta-aminopropionates, and 2-alkyl-imidazoline quaternary ammonium salts, and (3) mixtures thereof.

Methods of Modulating Microbiota and their Metabolic Outputs

In some aspects, the compositions or formulations disclosed herein, when contacted with a microbial community, (i) increase the abundance of at least one microbe in the microbial community, (ii) reduce the abundance of at least one microbe in the microbial community, (iii) are not metabolized by at least one microbe in the microbial community, and/or (iv) increase the production of beneficial metabolites by at least one microbe in the microbial community.

In some aspects, in the intestine, the oligosaccharides can (i) increase the abundance and/or function of beneficial bacteria such as bifidobacteria, (ii) increase the production of beneficial metabolites such as butyrate which fuel epithelial cells in the intestine, (iii) increase the production of beneficial metabolites such as acetate, butyrate and gamma-Aminobutyric acid which regulate immune responses to lower chronic inflammation, and/or (iv) increase the production of amino acids which may be required for mucin production. One or more of these actions may build or support the intestinal barrier, lower chronic inflammation in the intestinal barrier and systemically, and modulate gut-brain interactions.

Disclosed herein is a method for modulating a microbial community comprising at least one microorganism, the method comprising contacting the microbial community with a synthetic composition disclosed herein, wherein the at least one microorganism is modulated.

In some aspects, administration of a pharmaceutically acceptable composition of one or more of the compositions disclosed herein is employed to stimulate the growth of a microbe of interest, such as Bifidobacterium. In some aspects, administration of a pharmaceutically acceptable composition comprising at least one synthetic oligosaccharide is employed to increase the production of certain metabolites of interest, including short chain fatty acids, butyrate, lactate, or any combination thereof. Pharmaceutically acceptable salts, stereoisomers, and metabolites of one or more of the oligosaccharides described herein also are contemplated.

In some aspects, the oligosaccharides can stimulate the production of beneficial metabolites. In some aspects, those metabolites are short chain fatty acids (formate, acetate, propionate, butyrate, isobutyrate, 3-hydroxybutyrate, valerate, isovalerate). In some aspects, those metabolites are beneficial for brain health and cognition (gamma amino butyric acid, 3-hydroxybutyrate). In some aspects, the oligosaccharides can reduce the production of beneficial metabolites. In some aspects, those metabolites are branched chain amino acids (valine, leucine, isoleucine), and biogenic amines such as (putrescine, cadaverine, histamine). In some aspects, the oligosaccharides can stimulate the production of vitamins (Nicotinic Acid, Pantothenic Acid).

In some aspects the oligosaccharide compositions can modulate (enhance or reduce) the abundances of certain microbial communities. In some aspects, the targeted microbes are Bacteroidetes. In some aspects, those Bacteroidetes are Bacteroides unformis, Bacteroides fragilis, Prevotella, Prevotella copri. In some aspects, the targeted microbes are Firmicutes. In some aspects, the Firmicutes are Blautia, Eubacterium hallil, Faecalibacterium, Veillonella, Lactobacillus, Clostridiaceae, Clostridium butyricum, Roseburia, Roseburia intestinalis, Roseburia inulinivorans, Ruminococcus, Ruminococcus gnavus. In some aspects, the oligosaccharide compositions can reduce the abundance of Proteobacteria. In some aspects, those Proteobacteria are Klebsiella and Oxalobacter. In some aspects, the oligosaccharide compositions can modulate (enhance or reduce) the abundances of Bifidobacteria. In some aspects, the Bifidobacterium is pseudocatenulatum. In some aspects, the oligosaccharide compositions can modulate (enhance or reduce) the abundances of Verrucomicrobia. In some aspects, the Verrucomicorbia is Akkermansia.

In some aspects, the oligosaccharides or oligosaccharide compositions, or any mixture of oligosaccharides, is used to selectively stimulate growth of the one or more microbes. In some aspects, the microbes comprise probiotic microbes. In some aspects, the one or more microbes are in the gut of an animal, and the composition is administered to the animal. In some aspects, the one or more microbes (prebiotic microbes) is/are administered to the animal, either separately (e.g., sequentially) from the composition or simultaneously with the composition (e.g., administration of a composition comprising the probiotic microbe and one or a mixture of oligosaccharides. In some aspects the one or more microbes are in, or are introduced into a particular location or lumen (e.g., the vagina) of an animal or human. In some aspects, the probiotic microbe is Bifidobacterium pseudocatenulatum. In some aspects, the probiotic microbe is Lactobacillus Crispatus. In some aspects, the one or more microbes are soil microbes, oral microbes (e.g., bacteria), or skin microbes. In some aspects, the one or more oligosaccharides can be applied along with an antibiotic treatment. In some aspects, the one or more oligosaccharides can be applied along with an antibiotic treatment and one or more probiotic microbes. In some aspects, the one or more oligosaccharides can be applied along with a defined or undefined consortium of bacteria. In some aspects the one or more oligosaccharides can be used as an excipient.

In some aspects, the oligosaccharides as described herein, can be used to stimulate microbes of any sort. Examples of microbes that can be stimulated by the oligosaccharides include, for example, soil microbes (e.g., mycorrhizal fungi and bacteria and other microbes used as soil inoculants such as Azosprillum sp.), oral bacterial (e.g., Streptococcus mutans, Streptococcus gordonii, Streptococcus sanguis, and S. oralis) and skin bacteria (e.g., Propionibacterium acnes, also ammonia oxidizing bacteria, including but not limited to Nitrosomonas, Nitrosococcus, Nitrosospira, Nitrosocvstis, Nitrosolobus, and Nitrosovibrio.

Xyloglucan, and oligosaccharides derived therefrom, can be used for the selective growth of specific Bacteroides species, like B. ovatus (Larsbrink et al. 2014). It has been demonstrated that the xyloglycan utilization loci, with glycoside hydrolase genes, belongs to the families GH5 and GH31 which can be found in B. ovatus. The presence of these genes allow the growth of this species when used as a sole carbon source. Other major Bacteroides species in the gut like B. thetaiotaomicron, B. caccae or B. fragilis, lack this loci or part of it in their genomes, and, thusly, are unable to metabolize xyloglucan.

Curdlan, and oligosaccharides derived therefrom, can be used for the selective growth of specific Bacteroides species, like B. thetaiotaomicron or B. distasonis, when their genomes encode a specific type of glycoside hydrolase belonging to the family GH16. Orthologs of this gene are absent in the genomes of other Bacteroides species like B. caccae or B. ovatus, and are unable to grow on curdlan (Salyers et al. 1997).

β-glucan or lichenin, and oligosaccharides derived therefrom, can be used for the selective growth of specific Bacteroides species, like B. ovatus. This species encodes in its genome a specific type of GH16, with β 1-3,4 glucan activity (Tamura et al. 2017). It has been demonstrated that this polysaccharide enhances the growth of species of Firmicutes like Enterococcus faecium, Clostridium perfingens, Roseburia inulinivorans, and R. faecis (Beckmann et al. 2006, Sheridan et al. 2016).

Galactan, and oligosaccharides derived therefrom, can select for the growth of specific Bacteroides species, such as B. thetaiotaomicron, B. dorei and B. ovatus. Different types of endo-galactanases can be responsible for this selective growth, which belong to the families GH53 and GH147 (Lammerts van Bueren et al. 2017, Luis et al. 2018). The ability to consume galactan has also been described in some Bifidobacterial species (Biff breve, Bif longum, Bif long subsp. Infantis) (Hinz et al. 2005).

Mannan, and oligosaccharides derived therefrom, can selectively grow specific Bacteroides species, like B. fragilis or B. ovatus, which encode a GH26 endo-β1-4-mannosidase (Kawaguchi et al. 2014). This gene is absent in the genome of major intestinal species like B. thetaiotamicron, which are unable to grow on mannan or glucomannan. R. intestinalis and R. faecis can deplete mannan linkages (Leanti La Rosa et al. 2019), as well as members of Clostridium cluster XIVa (Desai et al. 2016, Sheridan et al. 2016), with GH26 encoded in their genomes. Also, GH26 has been characterized in specific species of Bifidobacteria, such as Bif. adolescentis (Kulcinskaja et al. 2013), confirming the ability of this species to grow on mannan. Galactomannan is consumed only by microorganism that encode endo-β1-4-mannosidase GH26 and alpha-galactosidase GH27 in their genomes, like B. ovatus, B. xylanisolvens (Reddy et al. 2016) or Roseburia intestinalis (Desai et al. 2016, Leanti La Rosa et al. 2019).

Xylan, arabinan and arabinoxylan, and oligosaccharides derived therefrom, can be used to selectively grow specific species of Bacteroides. Xylan can be metabolized by B. ovatus and B. unformis, while B. thetaiotaomicron or B. caccae are unable to grow in this substrate. Arabinan promotes the growth of B. thetaiotaomicron and B. ovatus, while arabinoxylan shows high selection for B. ovatus growth (Martens et al. 2011, Desai et al. 2016). It has been shown that strains of R. intestinalis, E. rectale and R. faecis can consume xylan or arabinoxylan as the sole carbon source (Desai et al. 2016, Sheridan et al. 2016). Certain bifidobacteria have the capacity to ferment xylan or arabinofuranosyl-containing oligosaccharides. Selective growth of B. adolescentis on xylose and arabinoxylan derived glycans was shown in vitro (Van Laere et al. 1999). Also, additional experiment confirmed that B. longum subsp. longum was also able to metabolize arabinoxylan (Margolles and De Los Reyes-Gavilin 2003).

### Therapeutics for Gastrointestinal Health

Inflammatory bowel diseases (IBDs) such as Crohn's Disease (CD) and ulcerative colitis (UC) are chronic relapsing diseases that lead to structural damage with destruction of the bowel wall.

Treatment of IBD follows a stepwise approach where the first step is administration of 5-aminosalicylates, which are local acting anti-inflammatories. These agents appear to have greater efficacy for the treatment of ulcerative colitis than for Crohn's disease, for which efficacy data are limited. If the patient's condition fails to respond to an adequate dose of 5-aminosalicylates, the second step is often corticosteroids, which tend to provide rapid relief of symptoms and a significant decrease in inflammation. If oral corticosteroid therapy fails, the third step is usually immunomodulators or anti-TNF therapy. Anti-TNF-α monoclonal antibody therapies are commonly highly effective, at least initially. However, subgroups of patients do not respond to therapy and other subgroups develop neutralizing antibodies. These patients show little or no change of clinical symptoms. Also, all patients are exposed to side effects of this type of therapy, such as infections, reactivation of tuberculosis, allergic reactions, skin disorders, demyelinating disorders, and lupus-like autoimmunity.

Mucosal healing, as visible by endoscopy, has recently emerged as a key treatment goal. Thus, the term “mucosal healing on endoscopy” has been developed to refer to visible resolution of ulcers in CD and erosions and ulcers in UC (Froslie et al. 2007; Markus et al. 2019).

There is some research which indicates that intestinal barrier function could be improved by oligosaccharides (Mirjam et al. 2009). One factor for IBD pathogenesis involves a defective intestinal barrier. This epithelial barrier function is impaired by inflammation; TNF-alpha and IFN-gamma are proinflammatory cytokines released during inflammation that further increase epithelial permeability at tight junctions. This defect leads to translocation of endotoxins and bacterial antigens, resulting in a persistent activation of the adaptive immune system. Increasing the relative abundance of Bifidobacteria in the gut, which can prevent LPS induced inflammation and pathogen colonization, is related to improved symptoms of IBD (Sartor et al. 2004). SCFA produced by specific gut microbes, like Clostridium butyricum (Kanoi et al. 2015), binds metabolite-sensing receptors, which develop key roles in the promotion of gut homeostasis and regulation of inflammatory responses (Cavaglieri et al. 2003). In addition, other microbial end products, like GABA or nicotic acid, can interact with host receptors, decreasing inflammation (Max et al. 2018; Li et al. 2017). Species that are generally capable of inducing local and systemic inflammation, like Proteobacteria, are related to IBD pathogenesis (Mukhopadhya et al. 2012).

Inflammatory conditions of the gastrointestinal tract include inflammatory bowel diseases (IBDs) such as Crohn's Disease (CD) and ulcerative colitis (UC). IBDs are chronic relapsing diseases that lead to structural damage with destruction of the bowel wall. Generally, treatment of these diseases follows a stepwise approach where the first step is administration of 5-aminosalicylates, which are local acting anti-inflammatories. These agents appear to have greater efficacy for the treatment of ulcerative colitis than for Crohn's disease, for which efficacy data are limited. If the patient's condition fails to respond to an adequate dose of 5-aminosalicylates, the second step is often corticosteroids, which tend to provide rapid relief of symptoms and a significant decrease in inflammation. However, a major goal is often to wean the patient off steroids as soon as possible to prevent long-term adverse effects from these agents. Most patients can tolerate a relatively rapid taper down in dose after a response is achieved. However occasionally, a very prolonged steroid taper is necessary to prevent relapse in patients who have had prolonged exposure to steroids in the past. If oral corticosteroid therapy fails, the third step is usually immunomodulators or anti-TNF therapy. Anti-TNF-α monoclonal antibody therapies highly effective; at least initially. However, subgroups of patients do not respond to therapy and other subgroups develop neutralizing antibodies. These patients show little or no change of clinical symptoms. Also, all patients are exposed to side effects of this type of therapy, such as infections, reactivation of tuberculosis, allergic reactions, skin disorders, demyelinating disorders, and lupus-like autoimmunity. For this reason, these therapies are usually only given to patients with severe symptoms or patients with a poor prognosis. Until recently, most treatments focused on inducing clinical remission, reducing diarrhea, and reducing abdominal pain. However, mucosal healing, as visible by endoscopy, has recently emerged as a key treatment goal (Neurath et al. Gut 61, 1619 (2012)). Thus, the term “mucosal healing on endoscopy” has been developed to refer to visible resolution of ulcers in CD and erosions and ulcers in UC (Froslie et al. Gastroenterology 133, 412 (2007)). Mucosal healing has been associated with more effective disease control, more frequent steroid-free remission of disease, lower rates of hospitalization and surgery, and improved quality of life as compared with conventional treatment goals. These findings highlight the role of mucosal healing for therapy of IBD and inflammatory gastrointestinal conditions in general. Although mucosal healing is an appealing goal for IBD treatment, it has not always been achievable and has exposed some patients to unnecessary risks, particularly when it has led to escalating drug therapies. Therefore, there is a for therapies which promote mucosal healing in inflammatory gastrointestinal conditions which are effective and safe with little or no adverse side effects, and which may be effectively used over the longer term.

In some aspects, the oligosaccharides, oligosaccharide compositions, or formulations thereof are useful as therapeutics in methods for treating, preventing, or improving conditions or disorders associated with gastrointestinal health. For example, in some aspects, disclosed herein are methods for the prevention or treatment of inflammatory conditions of the gastrointestinal tract, including inflammatory bowel disease (IBD), by administration of oligosaccharides, oligosaccharide compositions, or formulations thereof.

Disclosed herein is a method for treating or preventing a gastrointestinal condition or disease, the method comprising administering to a patient in need thereof a therapeutically effective amount of a synthetic composition disclosed herein, optionally wherein the synthetic composition is in the form of a pharmaceutical composition comprising a pharmaceutically acceptable carrier. In some aspects, the gastrointestinal condition or disease comprises inflammatory bowel disease, Crohn's disease, ulcerative colitis, or any combination thereof.

Gastrointestinal issues such as inflammatory conditions of the gastrointestinal tract include inflammatory bowel diseases (IBDs) such as Crohn's Disease (CD) and ulcerative colitis (UC). IBDs are chronic relapsing diseases that lead to structural damage with destruction of the bowel wall. While there exist some treatments, such treatments are not perfect and can be improved. Certain prebiotic and/or probiotic agents, such as oligosaccharides and/or beneficial microorganisms, may aid in treating or preventing gastrointestinal issues. There is a need in the art for oligosaccharides that can treat or prevent gastrointestinal issues, such as IBD, CD, and/or UC.

In some aspects, the disclosed oligosaccharide compositions are administered to a human or animal in need thereof. For example, in some aspects, the oligosaccharide compositions are administered to a person or animal having at least one condition selected from the group consisting of inflammatory bowel syndrome, constipation, diarrhea, colitis, Crohn's disease, colon cancer, functional bowel disorder (FBD), irritable bowel syndrome (IBS), excess sulfate reducing bacteria, inflammatory bowel disease (IBD), and ulcerative colitis. Irritable bowel syndrome (IBS) is characterized by abdominal pain and discomfort, bloating, and altered bowel function, constipation and/or diarrhea. There are three groups of IBS: Constipation predominant IBS (C-IBS), Alternating IBS (A-IBS) and Diarrhea predominant IBS (D-IBS). The oligosaccharide compositions are useful, e.g., for repressing or prolonging the remission periods on Ulcerative patients. The oligosaccharide compositions can be administered to treat or prevent any form of Functional Bowel Disorder (FBD), and in particular Irritable Bowel Syndrome (MS), such as Constipation predominant IBS (C-IBS), Alternating IBS (A-IBS) and Diarrhea predominant IBS (D-IBS); functional constipation and functional diarrhea. FBD is a general term for a range of gastrointestinal disorders which are chronic or semi-chronic and which are associated with bowel pain, disturbed bowel function and social disruption.

In some aspects, disclosed are novel therapeutic strategies for the treatment of inflammatory conditions of the gastrointestinal tract including IBD. In some aspects, the therapeutic method comprises contacting one or more microbes (e.g., bacteria, fungi, yeast) with a composition comprising one or a mixture of oligosaccharides.

In some aspects, disclosed is a method for the treatment of a chronic gastrointestinal condition associated with an impaired intestinal barrier function, the method comprising administering to a person having the chronic gastrointestinal condition an effective amount of an oligosaccharide selected from one or more of:
(i) an oligosaccharide having a generally linear poly-glucose chain including 1-3 glycosidic linkages,
(ii) an oligosaccharide a generally linear poly-xylose chain including beta 1-4 glycosidic linkages.
(iii) an oligosaccharide having a generally linear galactose backbone including beta 1-3 glycosidic linkages, and branches from the backbone comprising one or more galactose and/or arabinose including beta 1-6 galactose linkages and/or alpha 1-6 arabinose linkages,
(iv) an oligosaccharide having a generally linear poly-mannose backbone including beta 1-4 glycosidic linkages, with single galactose branches including alpha 1-4 glycosidic linkages,
(v) an oligosaccharide having a generally linear galactose backbone including beta 1-4 glycosidic linkages connected to a pectin fragment,
(vi) an oligosaccharide having a generally linear poly-glucose chain including beta 1-3 glycosidic and beta 1-4 glycosidic linkages,
(vii) an oligosaccharide having a generally linear poly-glucose backbone including beta 1-4 linkages, and single unit xylose branches linked to the backbone by alpha 1-6 bonds,
(viii) an oligosaccharide having a generally linear poly-xylose backbone including beta 1-4 glycosidic linkages, and short arabinose branches linked to the galactose through beta 1-2 linkages.

In some aspects, a treatment method is disclosed for the treatment of ulcerative colitis and/or Crohn's disease, the method comprising administering to a patient having ulcerative colitis and/or Crohn's disease an amount of oligosaccharides, oligosaccharide composition, or formulation thereof sufficient to induce remission by inducing and/or promoting mucosal healing in the patient. The oligosaccharides, oligosaccharide composition, or formulation thereof may be administered to the patient as sole therapy, in combination with an anti-inflammatory, or subsequent to administration of an anti-inflammatory. The patient may be an ulcerative colitis and/or Crohn's disease patient with moderate-to-severe symptoms who is receiving immunomodulator treatment.

In some aspects, a treatment method is disclosed for the treatment of irritable bowel syndrome, the method comprising administering to a patient having irritable bowel syndrome an amount of oligosaccharides, oligosaccharide composition, or formulation thereof sufficient to reduce visceral pain intensity, reduce visceral pain frequency, reduce visceral pain duration, and/or to normalize bowel movement in the patient. Further, the patient may be administered an amount of oligosaccharides, oligosaccharide composition, or formulation thereof sufficient to improve emotional state and/or mood of the patient. The oligosaccharides, oligosaccharide composition, or formulation thereof may be administered to the patient as sole therapy, in combination with a symptomatic medication such as an antispasmodic, an antidiarrheal, and/or a laxative, in combination with a Low-FODMAP diet, or subsequent to administration of a symptomatic medication or application of a Low-FODMAP diet.

In some aspects, disclosed is a method for reducing the risk of occurrence of a chronic gastrointestinal condition associated with an impaired intestinal barrier function, the method comprising administering to a person at risk of developing or re-developing the chronic gastrointestinal condition an effective amount of an oligosaccharide selected from one or more of:
(i) an oligosaccharide having a generally linear poly-glucose chain including 1-3 glycosidic linkages,
(ii) an oligosaccharide a generally linear poly-xylose chain including beta 1-4 glycosidic linkages.
(iii) an oligosaccharide having a generally linear galactose backbone including beta 1-3 glycosidic linkages, and branches from the backbone comprising one or more galactose and/or arabinose including beta 1-6 galactose linkages and/or alpha 1-6 arabinose linkages,
(iv) an oligosaccharide having a generally linear poly-mannose backbone including beta 1-4 glycosidic linkages, with single galactose branches including alpha 1-4 glycosidic linkages,
(v) an oligosaccharide having a generally linear galactose backbone including beta 1-4 glycosidic linkages connected to a pectin fragment,
(vi) an oligosaccharide having a generally linear poly-glucose chain including beta 1-3 glycosidic and beta 1-4 glycosidic linkages,
(vii) an oligosaccharide having a generally linear poly-glucose backbone including beta 1-4 linkages, and single unit xylose branches linked to the backbone by alpha 1-6 bonds,
(viii) an oligosaccharide having a generally linear poly-xylose backbone including beta 1-4 glycosidic linkages, and short arabinose branches linked to the galactose through beta 1-2 linkages.

In some aspects, disclosed is a treatment method for reducing the risk of relapse or for the elongation of the period of remission in a patient previously treated for ulcerative colitis and/or Crohn's disease, the method comprising administering to the patient an amount of oligosaccharides, oligosaccharide composition, or formulation thereof sufficient to promote mucosal healing and/or to maintain mucosal integrity in the patient. The oligosaccharides, oligosaccharide composition, or formulation thereof may be administered to the patient as sole intervention, in combination with an anti-inflammatory, or subsequent to administration of an anti-inflammatory. The patient may be an ulcerative colitis and/or Crohn's disease patient with moderate-to-severe symptoms who is receiving immunomodulator treatment.

In some aspects, disclosed is a method for reducing the risk of reoccurrence of irritable bowel syndrome and/or reducing the intensity of symptoms during reoccurrence of irritable bowel syndrome, the method comprising administering to a patient having irritable bowel syndrome an amount of oligosaccharides, oligosaccharide composition, or formulation thereof sufficient to improve intestinal barrier function. The oligosaccharides, oligosaccharide composition, or formulation thereof may be administered to the patient as sole therapy, in combination with a selected diet such as the Low-FODMAP diet.

In some aspects, a method for the treatment of a chronic gastrointestinal condition associated with an impaired intestinal barrier function is disclosed, the method comprising administering to a person having the chronic medical condition an effective amount of an oligosaccharide selected from one or more of:
(i) an oligosaccharide having a generally linear poly-glucose chain including 1-3 glycosidic linkages,
(ii) an oligosaccharide a generally linear poly-xylose chain including beta 1-4 glycosidic linkages.
(iii) an oligosaccharide having a generally linear galactose backbone including beta 1-3 glycosidic linkages, and branches from the backbone comprising one or more galactose and/or arabinose including beta 1-6 galactose linkages and/or alpha 1-6 arabinose linkages,
(iv) an oligosaccharide having a generally linear poly-mannose backbone including beta 1-4 glycosidic linkages, with single galactose branches including alpha 1-4 glycosidic linkages,
(v) an oligosaccharide having a generally linear galactose backbone including beta 1-4 glycosidic linkages connected to a pectin fragment,
(vi) an oligosaccharide having a generally linear poly-glucose chain including beta 1-3 glycosidic and beta 1-4 glycosidic linkages,
(vii) an oligosaccharide having a generally linear poly-glucose backbone including beta 1-4 linkages, and single unit xylose branches linked to the backbone by alpha 1-6 bonds,
(viii) an oligosaccharide having a generally linear poly-xylose backbone including beta 1-4 glycosidic linkages, and short arabinose branches linked to the galactose through beta 1-2 linkages.

In another aspect, a method for reducing the risk of occurrence of a chronic gastrointestinal condition associated with an impaired intestinal barrier function is disclosed, the method comprising administering to a person at risk of developing or re-developing the chronic medical condition an effective amount of an oligosaccharide selected from one or more of: (i) an oligosaccharide having a generally linear poly-glucose chain including 1-3 glycosidic linkages,
(ii) an oligosaccharide a generally linear poly-xylose chain including beta 1-4 glycosidic linkages.
(iii) an oligosaccharide having a generally linear galactose backbone including beta 1-3 glycosidic linkages, and branches from the backbone comprising one or more galactose and/or arabinose including beta 1-6 galactose linkages and/or alpha 1-6 arabinose linkages,
(iv) an oligosaccharide having a generally linear poly-mannose backbone including beta 1-4 glycosidic linkages, with single galactose branches including alpha 1-4 glycosidic linkages,
(v) an oligosaccharide having a generally linear galactose backbone including beta 1-4 glycosidic linkages connected to a pectin fragment,
(vi) an oligosaccharide having a generally linear poly-glucose chain including beta 1-3 glycosidic and beta 1-4 glycosidic linkages,
(vii) an oligosaccharide having a generally linear poly-glucose backbone including beta 1-4 linkages, and single unit xylose branches linked to the backbone by alpha 1-6 bonds,
(viii) an oligosaccharide having a generally linear poly-xylose backbone including beta 1-4 glycosidic linkages, and short arabinose branches linked to the galactose through beta 1-2 linkages.

In some aspects, disclosed are novel therapeutic strategies for the treatment of inflammatory conditions of the gastrointestinal tract including IBD. In some aspects, the therapeutic method comprises contacting one or more microbes (e.g., bacteria, fungi, yeast) with a composition or formulation comprising one or more of CLX101, CLX102, CLX103, CLX108, CLX109, CLX110, CLX111, CLX112, CLX113, CLX114, CLX115, CLX107, CLX115a, CLX117, CLX122, CLX123, CLX125, CLX126, CLX127, CLX128, CLX115AL, CLX122DS, CLX122DSF or CLX131, or any combination thereof, to (i) selectively stimulate growth of one or more microbes, (ii) increase the production of beneficial metabolites such as butyrate which fuel epithelial cells in the intestine, (iii) increase the production of beneficial metabolites such as acetate, butyrate and gamma-Aminobutyric acid which regulate immune responses to lower chronic inflammation, and/or (iv) increase the production of amino acids which may be required for mucin production. In some aspects, the microbe stimulated by the oligosaccharide(s) described herein is a probiotic microbe in the gut, comprising at least one of Bifidobacteria, Bifidobacterium pseudocatanatum, Bifidobacterium animalis, Bacteroides, Bacteroides ovatus, Firmicutes, Clostridium butyricum, Ruminococcus, Ruminococcus gnavus, Ruminococcus torque, Blautia, Roseburia, Faecalibacterium, or any combination thereof. One or more of CLX101, CLX102, CLX103, CLX108, CLX109, CLX110, CLX111, CLX112, CLX113, CLX114, CLX115, CLX107, CLX115a, CLX117, CLX122, CLX123, CLX125, CLX126, CLX127, CLX128, CLX115AL, CLX122DS, CLX122DSF or CLX131, or any combination thereof, can be combined with other ingredients to produce foodstuffs and supplements including infant formula, geriatric supplements, drinks, nutritional supplements, baking flours, and snack foods. One or more of these oligosaccharides can also be used as pharmaceutical products.

Thus, in some aspects, disclosed is the administration of a pharmaceutically acceptable composition of one or more of CLX101, CLX102, CLX103, CLX108, CLX109, CLX110, CLX111, CLX112, CLX113, CLX114, CLX115, CLX107, CLX115a, CLX117, CLX122, CLX123, CLX125, CLX126, CLX127, CLX128, CLX115AL, CLX122DS, CLX122DSF or CLX131, or any combination thereof, to stimulate the growth of Bifidobacteria in the gut, which can prevent LPS induced inflammation and pathogen colonization associated with IBD. In some aspects, disclosed is the administration of a pharmaceutically acceptable composition of one or more of CLX101, CLX102, CLX103, CLX108, CLX109, CLX110, CLX111, CLX112, CLX113, CLX114, CLX115, CLX107, CLX115a, CLX117, CLX122, CLX123, CLX125, CLX126, CLX127, CLX128, CLX115AL, CLX122DS, CLX122DSF or CLX131, or any combination thereof, to increase the production of butyrate. Pharmaceutically acceptable salts, stereoisomers, and metabolites of one or more of the oligosaccharides described herein also are contemplated.

In some aspects, administration of and administering a compound (e.g., oligosaccharide(s)) or composition thereof should be understood to mean providing a compound or salt thereof or a pharmaceutical composition comprising a compound. The compound or composition can be administered by another person to the patient (e.g., intravenously) or it can be self-administered by the subject (e.g., tablets or capsules). The term “patient” refers to mammals (for example, humans and veterinary animals such as dogs, cats, pigs, horses, sheep, and cattle). Administration of one or more of CLX101, CLX102, CLX103, CLX108, CLX109, CLX110, CLX111, CLX112, CLX113, CLX114, CLX115, CLX107, CLX115a, CLX117, CLX122, CLX123, CLX125, CLX126, CLX127, CLX128, CLX115AL, CLX122DS, CLX122DSF or CLX131, or any combination thereof, in combination with other compounds, such as excipients, colorants, fillers, binders, diluents, buffering agents, moistening agents, preservatives, flavoring agents, dyes, disintegrating agents, and pharmaceutically compatible carriers, is contemplated.

### Therapeutics for Cardiovascular Health

Cardiovascular disease can be caused by a variety of conditions such as obesity, obesity induced pre-diabetes, type 2 diabetes, and non-alcoholic fatty liver disease.

Most current therapeutic approaches aim at treating the consequences rather than causes of impaired metabolism associated with cardiovascular disease. This is not efficient and therefore, there remains a need for approaches that address potential causes or at least managing impaired metabolism over the longer term, and which are safe with little or no adverse side effects.

Prebiotics provide a solution. The intestinal microbiota participates in whole-body metabolism by affecting energy balance (Turnbaugh et al.2006), glucose metabolism (Cani et al. 2008) and the low-grade inflammation (Cani et al. 2009) associated with metabolic disorders. Intestinal microbiota derived lipopolysaccharide (LPS) is involved in the onset and progression of low-grade inflammation and plays a major role in the onset of disease (Cani et al. 2008). Further, altered cholesterol and bile acid metabolism as well as increased systemic inflammatory processes are risk factors for cardiovascular disease. Gut microbial composition is known to influence cholesterol regulation and hypercholesterolemia. This is due in part to a direct effect of microbes on cholesterol clearance via conversion of primary bile salts into excreted secondary bile acids. Indeed, microbes with an ability to hydrolyze bile salts into bile acids or dehydroxylate primary bile acids into secondary bile acids can increase the clearance of bile acids, increasing the demand for liver synthesis of bile from cholesterol, and in turn lowering plasma cholesterol levels. Bile acids of different structures may also play an endocrine role in decreasing inflammation and improving risk factors for cardiovascular disease (Kriaa A et al. 2019). Other microbial metabolites, like branched chain amino acids, are related to cardiovascular disease (Sikalidis A et al. 2020).

Short chain fatty acids (“SCFA”) produced by microbes also modulate chronic inflammation through alteration of intestinal barrier permeability and influence reverse cholesterol transport, also improving cardiovascular risk factors. Increases in SCFA production and SCFA producers, like Blautia, Faecalibacterium, Roseburia, Ruminococcus or Arkemansia, have been associated to a decrease in plasma cholesterol, increase in fecal excretion of bile acids, promoting the hepatic uptake of cholesterol from the blood (Chambers E. S et al. 2018).

Metabolic disorders include conditions such as obesity, obesity induced pre-diabetes, type 2 diabetes, and non-alcoholic fatty liver disease. Metabolic disorders are a rapidly growing, global epidemic. For example, the International Diabetes Federation (IDF) reports that as of 2013 there were more than 382 million people living with diabetes, and a further 316 million with impaired glucose tolerance who are at high risk from the disease. Since it is unlikely that there has been a dramatic alteration in genetic factors, environmental factors must play a key role in the rapid rise of metabolic disorders. One environmental factor is the intestinal microbiota with populations showing marked differences between healthy, obese, and type 2 diabetic patients (Qin et al. Nature 490, 55 (2012)). An altered composition and diversity of intestinal microbiota could play an important role in the development of metabolic disorders. The intestinal microbiota participates in whole-body metabolism by affecting energy balance (Turnbaugh et al. Nature 444, 1027 (2006)), glucose metabolism (Cani et al. Diabetes 57, 1470 (2008)) and the low-grade inflammation (Cani et al. Gut 58, 1091 (2009)) associated with metabolic disorders. Intestinal microbiota derived lipopolysaccharide (LPS) is involved in the onset and progression of low-grade inflammation and plays a major role in the onset of disease (Cani et al. Diabetes 57, 1470 (2008)). Most current therapeutic approaches aim at treating the consequences rather than causes of impaired metabolism. This is not efficient and therefore, there remains a need for approaches that address potential causes or at least managing impaired metabolism over the longer term, and which are safe with little or no adverse side effects.

In some aspects, disclosed are novel therapeutic strategies for the treatment of cardiovascular disease. In some aspects, the therapeutic method comprises contacting one or more microbes (e.g., bacteria, fungi, yeast) with a composition comprising one or a mixture of oligosaccharides, or a formulation thereof.

In some aspects, disclosed is a method for the treatment of or for reducing the risk of occurrence of, a chronic metabolic condition, the method comprising administering to a person having the chronic metabolic condition an effective amount of an oligosaccharide selected from one or more of:
(i) an oligosaccharide having a generally linear poly-glucose chain including 1-3 glycosidic linkages,
(ii) an oligosaccharide having a generally linear poly-glucose/mannose chain including interspersed beta 1-4 glucose and beta 1-4 mannose subunits,
(iii) an oligosaccharide a generally linear poly-xylose chain including beta 1-4 glycosidic linkages,
(iv) an oligosaccharide having a generally linear galactose backbone including beta 1-3 glycosidic linkages, and branches from the backbone comprising one or more galactose and/or arabinose including beta 1-6 galactose linkages and/or alpha 1-6 arabinose linkages,
(v) an oligosaccharide having a generally linear poly-mannose backbone including beta 1-4 glycosidic linkages, with single galactose branches including alpha 1-4 glycosidic linkages,
(vi) an oligosaccharide having a generally linear galactose backbone including beta 1-4 glycosidic linkages connected to a pectin fragment,
(vii) an oligosaccharide having a generally linear poly-glucose chain including interspersed beta 1-3 glycosidic and beta 1-4 glycosidic linkages,
(viii) an oligosaccharide having a generally linear poly-glucose chain including beta 1-3 glycosidic and beta 1-4 glycosidic linkages,
(ix) an oligosaccharide having a generally linear poly-glucose backbone including beta 1-4 linkages, and single unit xylose branches linked to the backbone by alpha 1-6 bonds,
(x) an oligosaccharide having a generally linear poly-xylose backbone including beta 1-4 glycosidic linkages, and short arabinose branches linked to the galactose through beta 1-2 linkages.

In some aspects, disclosed is a method for the treatment of, or for reducing the risk of occurrence of, a cardiovascular disease associated with elevated blood cholesterol, the method comprising administering to a patient having or at risk of the cardiovascular disease an amount of oligosaccharides, oligosaccharide compositions, or formulations thereof sufficient to lower blood cholesterol in the patient. The oligosaccharides, oligosaccharide compositions, or formulations thereof may be administered to the patient as sole therapy, in combination with a cholesterol-lowering agent, or subsequent to administration of a cholesterol-lowering agent. The cholesterol lowering agent may be a statin, a plant sterol, and/or a plant stanol.

In some aspects, disclosed is a method for the treatment of, or for reducing the risk of occurrence of, a cardiovascular disease associated with an elevated low-density lipoprotein (LDL) cholesterol/high-density lipoprotein (HDL) cholesterol ratio, the method comprising administering to a patient having or at risk of the cardiovascular disease an amount of oligosaccharides, oligosaccharide compositions, or formulations thereof sufficient to reduce the LDL/HDL ratio in the patient. The oligosaccharides, oligosaccharide compositions, or formulations thereof may be administered to the patient as sole therapy, in combination with a low-density lipoprotein (LDL) cholesterol lowering agent, or a high-density lipoprotein (HDL) cholesterol increasing agent, or subsequent to administration of such an agent. The agent may be an omega-3 fatty acid such as DHA.

In some aspects, disclosed is a method for the treatment of, or for reducing the risk of occurrence of, a cardiovascular disease associated with platelet activity, the method comprising administering to a patient having or at risk of the cardiovascular disease an amount of oligosaccharides, oligosaccharide compositions, or formulations thereof sufficient to reduce platelet activity in the patient. The oligosaccharides, oligosaccharide compositions, or formulations thereof may be administered to the patient as sole therapy, in combination with a platelet lowering agent, or subsequent to administration of such an agent. The agent may be an omega-3 fatty acid such as DHA.

In some aspects, the oligosaccharides, oligosaccharide compositions, or formulations thereof are administered in an amount to improve vascular function, lower heart rate and/or blood pressure.

In some aspects, disclosed is a method for the treatment of, or for reducing the risk of occurrence of, type II diabetes, the method comprising administering to a patient having or at risk of type II diabetes an amount of oligosaccharides, oligosaccharide compositions, or formulations thereof sufficient to lower post-prandial plasma glucose in the patient. The oligosaccharides, oligosaccharide compositions, or formulations thereof may be administered to the patient as sole therapy, in combination with a long-chain fiber, or subsequent to administration of a long-chain fiber. The long-chain fiber may be a β-glucan oat fiber or a barley fiber.

In some aspects, disclosed is a method for the treatment of, or for reducing the risk of occurrence of, type II diabetes, the method comprising administering to a patient having or at risk of type II diabetes an amount of oligosaccharides, oligosaccharide compositions, or formulations thereof sufficient to improve glycemic control as measured by Hemoglobin A1c in the patient. The oligosaccharides, oligosaccharide compositions, or formulations thereof may be administered to the patient as sole therapy, in combination with an antidiabetic agent, or subsequent to administration of an antidiabetic agent. The antidiabetic agent may be a β-insulin-secreting agent.

In some aspects, disclosed is a method for the treatment of, or for reducing the risk of occurrence of, a chronic metabolic condition associated with chronic systemic inflammation, the method comprising administering to a patient having or at risk of the condition an amount of oligosaccharides, oligosaccharide compositions, or formulations thereof sufficient to lower chronic systemic inflammation in the patient. The patient at risk of a chronic metabolic condition may be an obese patient or a prediabetic patient.

In some aspects, the oligosaccharides, oligosaccharide compositions, or formulations thereof are useful as therapeutics in methods for treating, preventing, or improving conditions or disorders associated with cardiovascular health. For example, in some aspects, disclosed herein are methods for the prevention or treatment of cardiovascular disease by administration of oligosaccharides, oligosaccharide compositions, or formulations thereof.

In some aspects, disclosed is novel therapeutic strategies for the treatment of cardiovascular disease. In some aspects, the therapeutic method comprises contacting one or more microbes (e.g., bacteria, fungi, yeast) with a composition comprising one or more of CLX101, CLX102, CLX103, CLX108, CLX109, CLX110, CLX111, CLX112, CLX113, CLX114, CLX115, CLX107, CLX115a, CLX117, CLX122, CLX123, CLX125, CLX126, CLX127, CLX128, CLX115AL, CLX122DS, CLX122DSF or CLX131, or any combination thereof, to (1) selectively stimulate growth of one or more microbes with the ability to hydrolyze bile salts into bile acids or dehydroxylate primary bile acids into secondary bile acids and/or (2) increase the production of beneficial metabolites such as SCFA. In some aspects, the microbe stimulated by the oligosaccharide(s) described herein is a microbe in the gut, comprising at least one of Lactobacillus crispatus, Lactobacillus rhamnosus, Bifidobacteria, Bifidobacterium pseudocatenulatum, Bifidobacterium longum subsp. longum, Bifidobacterium animalis, Firmicutes, Clostridium butyricum, Ruminococcus, Ruminococcus gnavus, Blautia, Roseburia, Faecalibacterium, Ruminococcus or Arkemansia, or any combination thereof. One or more of CLX101, CLX102, CLX103, CLX108, CLX109, CLX110, CLX111, CLX112, CLX113, CLX114, CLX115, CLX107, CLX115a, CLX117, CLX122, CLX123, CLX125, CLX126, CLX127, CLX128, CLX115AL, CLX122DS, CLX122DSF or CLX131, or any combination thereof, can be combined with other ingredients to produce foodstuffs and supplements including infant formula, geriatric supplements, drinks, nutritional supplements, baking flours, and snack foods. One or more of these oligosaccharides can also be used as pharmaceutical products.

Thus, in some aspects, disclosed is the administration of a pharmaceutically acceptable composition of one or more of CLX101, CLX102, CLX103, CLX108, CLX109, CLX110, CLX111, CLX112, CLX113, CLX114, CLX115, CLX107, CLX115a, CLX117, CLX122, CLX123, CLX125, CLX126, CLX127, CLX128, CLX115AL, CLX122DS, CLX122DSF or CLX131, or any combination thereof, to stimulate the growth of Blautia, Faecalibacterium, Roseburia, Ruminococcus or Arkemansia, or any combination thereof for the purpose(s) of decreasing in plasma cholesterol, increasing in fecal excretion of bile acids, and/or promoting the hepatic uptake of cholesterol from the blood. Also disclosed is the administration of a pharmaceutically acceptable composition of one or more of CLX101, CLX102, CLX103, CLX108, CLX109, CLX110, CLX111, CLX112, CLX113, CLX114, CLX115, CLX107, CLX115a, CLX117, CLX122, CLX123, CLX125, CLX126, CLX127, CLX128, CLX115AL, CLX122DS, CLX122DSF or CLX131, or any combination thereof, to increase the production of SCFA. Pharmaceutically acceptable salts, stereoisomers, and metabolites of one or more of the oligosaccharides described herein also are contemplated.

In some aspects, administration of and administering a compound (e.g., oligosaccharide(s)) or composition should be understood to mean providing a compound or salt thereof or a pharmaceutical composition comprising a compound. The compound or composition can be administered by another person to the patient (e.g., intravenously) or it can be self-administered by the subject (e.g., tablets or capsules). The term “patient” refers to mammals (for example, humans and veterinary animals such as dogs, cats, pigs, horses, sheep, and cattle). Administration of one or more of CLX101, CLX102, CLX103, CLX108, CLX109, CLX110, CLX111, CLX112, CLX113, CLX114, CLX115, CLX107, CLX115a, CLX117, CLX122, CLX123, CLX125, CLX126, CLX127, CLX128, CLX115AL, CLX122DS, CLX122DSF or CLX131, or any combination thereof, in combination with other compounds, such as excipients, colorants, fillers, binders, diluents, buffering agents, moistening agents, preservatives, flavoring agents, dyes, disintegrating agents, and pharmaceutically compatible carriers, is contemplated.

### Therapeutics for Renal System Health

Chronic kidney disease (CKD) is a general term for heterogeneous disorders affecting the structure and function of the kidneys. CKD is associated with significant rates of morbidity, mortality, and healthcare costs. The mean global prevalence of CKD has been estimated at 13.4%. Progressive renal failure results in higher concentrations of urea in blood.

There is no cure for CKD. The main treatments are lifestyle changes, medication to control associated problems, such as high blood pressure and high cholesterol, dialysis, and kidney transplant. Therefore, there remains a need for a safe and effective way of addressing potential causes of chronic kidney diseases or at least managing the condition over the longer term.

Exposure of intestinal bacteria to urea through gastrointestinal secretions results in the conversion of urea to ammonia via bacterial urease. This high concentration of urea causes overgrowth of bacterial families containing urease. Expansion of bacterial families producing uricase and indole- and p-cresyl-forming enzymes occurs in patients with end-stage renal disease (ESRD) compared with healthy controls. The resulting intestinal-derived uremic toxins, such as P-cresyl sulfate (PCS), indoxyl sulfate (IS), and trimethylamine N-oxide (TMAO), have been implicated in the progression of CKD and an increased cardiovascular risk. These toxins can damage the epithelial barrier and induce chronic, low-grade inflammation (Hobby et al. 2019).

The normal gut microbiota can protect the kidney whereas gut dysbiosis can facilitate CKD development. The gut dysbiosis in CKD is associated to reduction of genera such as Bifidobacterium, Lactobacillus and Prevotella are reduced, and an increase on species that are generally capable of inducing local and systemic inflammation, like Proteobacteria (Ren Z. et al. 2020; Kanbay et al. 2018). The presence of anti-inflammatory substances such as SCFA (Wong et al. 2014) or GABA (Barrett et al. 2012) is associated to CKD microbiome dysbiosis.

Chronic kidney disease (CKD) is a general term for heterogeneous disorders affecting the structure and function of the kidneys. CKD is associated with significant rates of morbidity, mortality, and healthcare costs. The mean global prevalence of CKD has been estimated at 13.4%. Progressive renal failure results in higher concentrations of urea in blood. Exposure of intestinal bacteria to urea through gastrointestinal secretions results in the conversion of urea to ammonia via bacterial urease. This high concentration of urea causes overgrowth of bacterial families containing urease. Expansion of bacterial families producing uricase and indole- and p-cresyl-forming enzymes occurs in patients with end-stage renal disease (ESRD) compared with healthy controls. The resulting intestinal-derived uremic toxins, such as P-cresyl sulfate (PCS), indoxyl sulfate (IS), and trimethylamine N-oxide (TMAO), have been implicated in the progression of CKD and an increased cardiovascular risk. These toxins can damage the epithelial barrier and induce chronic, low-grade inflammation. Also, the intestinal microbiota of patients with ESRD, is different from that of healthy controls. For example, in CKD patients, genera such as Klebsiella and Enterobacteriaceae are enriched while genera such as Bifidobacterium, Lactobacillus, Blautia and Roseburia are reduced (Ren et al, Advanced Science, 7,20, (2020)). There is no cure for CKD. The main treatments are lifestyle changes, medication to control associated problems, such as high blood pressure and high cholesterol, dialysis, and kidney transplant. Therefore there remains a need for a safe and effective way of addressing potential causes of chronic kidney diseases or at least managing the condition over the longer term.

In some aspects, disclosed herein are novel therapeutic strategies for the treatment of chronic kidney disease. In some aspects, the therapeutic methods comprise contacting one or more microbes (e.g., bacteria, fungi, yeast) with a composition comprising one or a mixture of oligosaccharides.

In some aspects, disclosed is a method for the treatment of or for reducing the risk of occurrence of, a chronic kidney condition, the method comprising administering to a person having the chronic kidney condition an effective amount of an oligosaccharide selected from one or more of:
(i) an oligosaccharide having a generally linear poly-glucose chain including 1-3 glycosidic linkages,
(ii) an oligosaccharide having a generally linear poly-glucose/mannose chain including interspersed beta 1-4 glucose and beta 1-4 mannose subunits,
(iii) an oligosaccharide a generally linear poly-xylose chain including beta 1-4 glycosidic linkages,
(iv) an oligosaccharide having a generally linear galactose backbone including beta 1-3 glycosidic linkages, and branches from the backbone comprising one or more galactose and/or arabinose including beta 1-6 galactose linkages and/or alpha 1-6 arabinose linkages,
(v) an oligosaccharide having a generally linear poly-mannose backbone including beta 1-4 glycosidic linkages, with single galactose branches including alpha 1-4 glycosidic linkages,
(vi) an oligosaccharide having a generally linear galactose backbone including beta 1-4 glycosidic linkages connected to a pectin fragment,
(vii) an oligosaccharide having a generally linear poly-glucose chain including interspersed beta 1-3 glycosidic and beta 1-4 glycosidic linkages,
(viii) an oligosaccharide having a generally linear poly-glucose chain including beta 1-3 glycosidic and beta 1-4 glycosidic linkages,
(ix) an oligosaccharide having a generally linear poly-glucose backbone including beta 1-4 linkages, and single unit xylose branches linked to the backbone by alpha 1-6 bonds,
(x) an oligosaccharide having a generally linear poly-xylose backbone including beta 1-4 glycosidic linkages, and short arabinose branches linked to the galactose through beta 1-2 linkages.

In some aspects, disclosed is a method for the treatment of, or for reducing the risk of occurrence of, poor glycemic control in a chronic kidney condition, the method comprising administering to a patient having the chronic kidney condition an amount of oligosaccharides, oligosaccharide compositions, or formulations thereof sufficient to improve glycemic control as measured by Hemoglobin A1c in the patient. The oligosaccharides, oligosaccharide compositions, or formulations thereof may be administered to the patient as sole therapy, in combination with an antidiabetic agent, or subsequent to administration of an antidiabetic agent. The antidiabetic agent may be a β-insulin-secreting agent. Preferably the patient is a stage 2 to stage 4 patient.

In some aspects, disclosed is a method for the treatment of, or for reducing the risk of occurrence of, hypertension in a chronic kidney condition, the method comprising administering to a patient having the chronic kidney condition an amount of oligosaccharides, oligosaccharide compositions, or formulations thereof sufficient to lower blood pressure in the patient. The oligosaccharides, oligosaccharide compositions, or formulations thereof may be administered to the patient as sole therapy, in combination with an antihypertensive agent, or subsequent to administration of an antihypertensive agent. The antihypertensive agent may be an angiotensin-converting enzyme (ACE) inhibitor or angiotensin II receptor blocker (ARB). Preferably the patient is a stage 2 to stage 4 patient.

In some aspects, disclosed is a method for the treatment of, or for reducing the risk of occurrence of, a chronic kidney condition associated with chronic systemic inflammation, the method comprising administering to a patient having or at risk of the condition an amount of oligosaccharides, oligosaccharide compositions, or formulations thereof sufficient to lower chronic systemic inflammation in the patient.

In some aspects, the oligosaccharides, oligosaccharide compositions, or formulations thereof are useful as therapeutics in methods for treating, preventing, or improving conditions or disorders associated with renal system health. For example, in some aspects, disclosed herein are methods for the prevention or treatment of chronic kidney disease (CKD) by administration of oligosaccharides, oligosaccharide compositions, or formulations thereof.

In some aspects, disclosed are novel therapeutic strategies for the treatment of chronic kidney disease. In some aspects, the therapeutic method comprises contacting one or more microbes (e.g., bacteria, fungi, yeast) with a composition comprising one or more CLX101, CLX102, CLX103, CLX108, CLX109, CLX110, CLX111, CLX112, CLX113, CLX114, CLX115, CLX107, CLX115a, CLX117, CLX122, CLX123, CLX125, CLX126, CLX127, CLX128, CLX115AL, CLX122DS, CLX122DSF or CLX131, or any combination thereof, to selectively stimulate growth of (i) one or more microbes and/or (ii) increase the production of beneficial metabolites such as GABA and SCFA. In some aspects, the microbe stimulated by the oligosaccharide(s) described herein is a microbe in the gut, comprising at least one of Lactobacillus crispatus, Lactobacillus rhamnosus, Bifidobacteria, Bifidobacterium pseudocatenulatum, Bifidobacterium longum subsp. longum, Bifidobacterium animalis, Firmicutes, Clostridium butyricum, Ruminococcus, Ruminococcus gnavus, Blautia, Roseburia, Prevotella or any combination thereof. One or more of CLX101, CLX102, CLX103, CLX108, CLX109, CLX110, CLX111, CLX112, CLX113, CLX114, CLX115, CLX107, CLX115a, CLX117, CLX122, CLX123, CLX125, CLX126, CLX127, CLX128, CLX115AL, CLX122DS, CLX122DSF or CLX131, or any combination thereof, can be combined with other ingredients to produce foodstuffs and supplements including infant formula, geriatric supplements, drinks, nutritional supplements, baking flours, and snack foods. One or more of these oligosaccharides can also be used as pharmaceutical products.

Thus, in some aspects, disclosed is the administration of a pharmaceutically acceptable composition of one or more of CLX101, CLX102, CLX103, CLX108, CLX109, CLX110, CLX111, CLX112, CLX113, CLX114, CLX115, CLX107, CLX115a, CLX117, CLX122, CLX123, CLX125, CLX126, CLX127, CLX128, CLX115AL, CLX122DS, CLX122DSF or CLX131, or any combination thereof, to stimulate the growth of Bifidobacterium, Lactobacillus, Prevotella, or any combination thereof. In some aspects, disclosed is the administration of a pharmaceutically acceptable composition of one or more of CLX101, CLX102, CLX103, CLX108, CLX109, CLX110, CLX111, CLX112, CLX113, CLX114, CLX115, CLX107, CLX115a, CLX117, CLX122, CLX123, CLX125, CLX126, CLX127, CLX128, CLX115AL, CLX122DS, CLX122DSF or CLX131, or any combination thereof, to increase the production of anti-inflammatory metabolites such as GABA and SCF. Pharmaceutically acceptable salts, stereoisomers, and metabolites of one or more of the oligosaccharides described herein also are contemplated.

In some aspects, disclosed is administration of and administering a compound (e.g., oligosaccharide(s)) or composition should be understood to mean providing a compound or salt thereof or a pharmaceutical composition comprising a compound. The compound or composition can be administered by another person to the patient (e.g., intravenously) or it can be self-administered by the subject (e.g., tablets or capsules). The term “patient” refers to mammals (for example, humans and veterinary animals such as dogs, cats, pigs, horses, sheep, and cattle). Administration of one or more of CLX101, CLX102, CLX103, CLX108, CLX109, CLX110, CLX111, CLX112, CLX113, CLX114, CLX115, CLX107, CLX115a, CLX117, CLX122, CLX123, CLX125, CLX126, CLX127, CLX128, or CLX131, or any combination thereof, in combination with other compounds, such as excipients, colorants, fillers, binders, diluents, buffering agents, moistening agents, preservatives, flavoring agents, dyes, disintegrating agents, and pharmaceutically compatible carriers, is contemplated.

### Therapeutics for Central Nervous System Health

Disorders of gut-brain interactions (formerly known as functional gastrointestinal disorders) are a group of conditions that have no organic explanation for their symptoms. Notable examples include irritable bowel syndrome, functional dyspepsia, and functional constipation. These conditions affect up to 40% of people at any one point in time, and two-thirds of these people will have chronic, fluctuating symptoms. The pathophysiology of disorders of gut-brain interactions is complex but involves bidirectional dysregulation of gut-brain interaction, microbial dysbiosis within the intestine, altered mucosal immune function, visceral hypersensitivity, and abnormal gastrointestinal motility. Psychological comorbidity is common.

These conditions are difficult to treat, and they have a considerable impact on health-care systems. There is no cure for disorders of gut-brain interactions such as IBS and treatment options generally target individual symptoms. This is partly related to incomplete understanding of the underlying pathophysiology. For example, the current first-line therapies for IBS target the predominant symptom and mainly affect one symptom in the symptom complex. For patients with IBS-D, the m-opioid receptor agonist Loperamide is recommended, although the available evidence of efficacy is limited. For patients with IBS-C, increasing the intake of dietary fiber, for example, psyllium husk, or use of osmotic laxatives, such as polyethylene glycol (PEG), is prescribed. These approaches may be associated with a beneficial effect on one symptom, but effects on other IBS symptoms are less prominent. For this reason, many patients adopt a diet which is low in fermentable oligosaccharides, disaccharides, monosaccharides and polyols (FODMAPs). These carbohydrates can be incompletely absorbed in the small intestine and pass into the large intestine where they are fermented by intestinal bacteria, leading to gas production and bloating. Also, they may stimulate motility by causing a net flux of water into the lumen. Clinical trials suggest that some patients have a favorable, short-term response to a low-FODMAP diet. However, even short-term use of the low-FODMAP diet has been associated with potentially unfavorable changes in intestinal microbiota composition. Also, the diet is difficult to comply with over the long term and proper application requires the gradual reintroduction of some sources of FODMAPs because many of these sources (e.g., fruits and vegetables) are important for healthy nutrition. Upon reintroduction of FODMAPs, the symptoms often return. Patients are therefore left with the choice of remaining on a difficult diet which is potentially unhealthy over the long term, or reintroducing foods which may trigger symptoms. Therefore, a need remains for a generally safe and effective way for fundamentally addressing potential causes of disorders of gut-brain interactions, at least managing these conditions over the longer term.

Gut-brain connections may be driven by constant bidirectional flux of metabolites between the gut microorganisms and the host, and there is evidence that supports that neurological states may be impacted by gut composition and their metabolic output. Amino acid metabolism by the intestinal microbiome can lead to neurotransmitter production. Metabolism of glutamate by some gut bacteria, can increase GABA levels in the gut, which is inverse related to depression, bipolar disorder, schizophrenia and ASD. Aromatic metabolites can be metabolized by some gut microorganisms into a large group of downstream neurotransmitters, like catecholamines, dopamine and noradrenaline, with neuroactive properties. In addition, SCFA production by the microbiota, plays an important role in lowering systemic inflammation that can lead to reduced neuroinflammation (Needham et al. 2020). Furthermore, high abundance of Bifidobacteria in the gut, which is linked to reduced LPS levels, can lead to a reduction of neuroinflammation and depression-like behavior (Kim et al. 2018).

Disorders of gut-brain interactions (formerly known as functional gastrointestinal disorders) are a group of conditions that have no organic explanation for their symptoms. Notable examples include irritable bowel syndrome, functional dyspepsia, and functional constipation. These conditions affect up to 40% of people at any one point in time, and two-thirds of these people will have chronic, fluctuating symptoms. The pathophysiology of disorders of gut-brain interactions is complex but involves bidirectional dysregulation of gut-brain interaction, microbial dysbiosis within the intestine, altered mucosal immune function, visceral hypersensitivity, and abnormal gastrointestinal motility. Psychological comorbidity is common. These conditions are difficult to treat, and they have a considerable impact on health-care systems. There is no cure for disorders of gut-brain interactions such as IBS and treatment options generally target individual symptoms. This is partly related to incomplete understanding of the underlying pathophysiology. For example, the current first-line therapies for IBS target the predominant symptom and mainly affect one symptom in the symptom complex. For patients with IBS-D, the m-opioid receptor agonist Loperamide is recommended, although the available evidence of efficacy is limited. For patients with IBS-C, increasing the intake of dietary fiber, for example, psyllium husk, or use of osmotic laxatives, such as polyethylene glycol (PEG), is prescribed. These approaches may be associated with a beneficial effect on one symptom, but effects on other IBS symptoms are less prominent. For this reason, many patients adopt a diet which is low in fermentable oligosaccharides, disaccharides, monosaccharides and polyols (FODMAPs). These carbohydrates can be incompletely absorbed in the small intestine and pass into the large intestine where they are fermented by intestinal bacteria, leading to gas production and bloating. Also, they may stimulate motility by causing a net flux of water into the lumen. Clinical trials suggest that some patients have a favorable, short-term response to a low-FODMAP diet. However, even short-term use of the low-FODMAP diet has been associated with potentially unfavorable changes in intestinal microbiota composition. Also, the diet is difficult to comply with over the long term and proper application requires the gradual reintroduction of some sources of FODMAPs because many of these sources (e.g., fruits and vegetables) are important for healthy nutrition. Upon reintroduction of FODMAPs, the symptoms often return. Patients are therefore left with the choice of remaining on a difficult diet which is potentially unhealthy over the long term, or reintroducing foods which may trigger symptoms. Therefore, a need remains for a generally safe and effective way for fundamentally addressing potential causes of disorders of gut-brain interactions, at least managing these conditions over the longer term.

In some aspects, disclosed are novel therapeutic strategies for the treatment of nervous system disorders. In some aspects, the therapeutic method comprises contacting one or more microbes (e.g., bacteria, fungi, yeast) with a composition comprising one or a mixture of oligosaccharides.

In some aspects, disclosed is a method for the treatment of, or for reducing the risk of occurrence of, a chronic medical condition associated with dysfunction in gut brain interactions, the method comprising administering to a person having the chronic medical condition an effective amount of an oligosaccharide selected from one or more of:
(i) an oligosaccharide having a generally linear poly-glucose chain including 1-3 glycosidic linkages,
(ii) an oligosaccharide having a generally linear poly-glucose/mannose chain including interspersed beta 1-4 glucose and beta 1-4 mannose subunits,
(iii) an oligosaccharide a generally linear poly-xylose chain including beta 1-4 glycosidic linkages,
(iv) an oligosaccharide having a generally linear galactose backbone including beta 1-3 glycosidic linkages, and branches from the backbone comprising one or more galactose and/or arabinose including beta 1-6 galactose linkages and/or alpha 1-6 arabinose linkages,
(v) an oligosaccharide having a generally linear poly-mannose backbone including beta 1-4 glycosidic linkages, with single galactose branches including alpha 1-4 glycosidic linkages,
(vi) an oligosaccharide having a generally linear galactose backbone including beta 1-4 glycosidic linkages connected to a pectin fragment,
(vii) an oligosaccharide having a generally linear poly-glucose chain including interspersed beta 1-3 glycosidic and beta 1-4 glycosidic linkages,
(viii) an oligosaccharide having a generally linear poly-glucose chain including beta 1-3 glycosidic and beta 1-4 glycosidic linkages,
(ix) an oligosaccharide having a generally linear poly-glucose backbone including beta 1-4 linkages, and single unit xylose branches linked to the backbone by alpha 1-6 bonds,
(x) an oligosaccharide having a generally linear poly-xylose backbone including beta 1-4 glycosidic linkages, and short arabinose branches linked to the galactose through beta 1-2 linkages.

In some aspects, disclosed is a method for treating, or reducing the risk of occurrence of an emotion and/or mood disorder in a patient, the method comprising administering to the patient an effective amount of oligosaccharides, oligosaccharide compositions, or formulations thereof. The patient may suffer from stress, and/or an impaired intestinal barrier.

In some aspects, the oligosaccharides, oligosaccharide compositions, or formulations thereof are useful as therapeutics in methods for treating, preventing, or improving conditions or disorders associated with central nervous system health.

In some aspects, disclosed are novel therapeutic strategies for the treatment of nervous system disorders. In some aspects, the therapeutic method comprises contacting one or more microbes (e.g., bacteria, fungi, yeast) with a composition comprising one or a mixture of CLX101, CLX 102, CLX 103, CLX105, CLX108, CLX109, CLX110, CLX111, CLX112, CLX113, CLX114, CLX115, CLX107, CLX115a, CLX116, CLX117, CLX118, CLX119, CLX122, CLX123, CLX124, CLX124, CLX126, CLX127, CLX128, CLX129, CLX130, CLX131, CLX132, CLX115AL, CLX122DS, CLX122DSF and CLX133 to (i) selectively stimulate growth of one or more microbes and/or (ii) increase the production of beneficial metabolites such as GABA, SCFA, and aromatic metabolites. In some aspects, the microbe stimulated by the oligosaccharide(s) described herein is a microbe in the gut, such as Lactobacillus crispatus, Lactobacillus rhamnosus, Bifidobacteria, Bifidobacterium pseudocatenulatum, Bifidobacterium longum subsp. longum, Bifidobacterium animalis, Firmicutes, Clostridium butyricum, Ruminococcus, Ruminococcus gnavus, Blautia, Roseburia, or any combinations thereof. One or more of CLX101, CLX 102, CLX 103, CLX105, CLX108, CLX109, CLX110, CLX111, CLX112, CLX113, CLX114, CLX115, CLX107, CLX115a, CLX116, CLX117, CLX118, CLX119, CLX122, CLX123, CLX124, CLX124, CLX126, CLX127, CLX128, CLX129, CLX130, CLX131, CLX132, CLX115AL, CLX122DS, CLX122DSF and CLX133 can be combined with other ingredients to produce foodstuffs and supplements including infant formula, geriatric supplements, drinks, nutritional supplements, baking flours, and snack foods. One or more of these oligosaccharides can also be used as pharmaceutical products.

Thus, in some aspects, disclosed is the administration of a pharmaceutically acceptable composition of one or more CLX101, CLX 102, CLX 103, CLX105, CLX108, CLX109, CLX110, CLX111, CLX112, CLX113, CLX114, CLX115, CLX107, CLX115a, CLX116, CLX117, CLX118, CLX119, CLX122, CLX123, CLX124, CLX124, CLX126, CLX127, CLX128, CLX129, CLX130, CLX131, CLX132, CLX115AL, CLX122DS, CLX122DSF and CLX133 to increase the growth of Bifidobacterium for the treatment of irritable bowel syndrome. In some aspects, disclosed is the administration of a pharmaceutically acceptable composition of one or more CLX101, CLX 102, CLX 103, CLX105, CLX108, CLX109, CLX110, CLX111, CLX112, CLX113, CLX114, CLX115, CLX107, CLX115a, CLX116, CLX117, CLX118, CLX119, CLX122, CLX123, CLX124, CLX124, CLX126, CLX127, CLX128, CLX129, CLX130, CLX131, CLX132, CLX115AL, CLX122DS, CLX122DSF and CLX133 to increase the production of SCFA. Pharmaceutically acceptable salts, stereoisomers, and metabolites of one or more of the oligosaccharides described herein also are contemplated.

In some aspects, administration of and administering a compound (e.g., oligosaccharide(s)) or composition should be understood to mean providing a compound or salt thereof or a pharmaceutical composition comprising a compound. The compound or composition can be administered by another person to the patient (e.g., intravenously) or it can be self-administered by the subject (e.g., tablets or capsules). The term “patient” refers to mammals (for example, humans and veterinary animals such as dogs, cats, pigs, horses, sheep, and cattle). Administration of one or more of CLX101, CLX 102, CLX 103, CLX105, CLX108, CLX109, CLX110, CLX111, CLX112, CLX113, CLX114, CLX115, CLX107, CLX115a, CLX116, CLX117, CLX118, CLX119, CLX122, CLX123, CLX124, CLX124, CLX126, CLX127, CLX128, CLX129, CLX130, CLX131, CLX132, CLX115AL, CLX122DS, CLX122DSF and CLX133 in combination with other compounds, such as excipients, colorants, fillers, binders, diluents, buffering agents, moistening agents, preservatives, flavoring agents, dyes, disintegrating agents, and pharmaceutically compatible carriers, is contemplated.

### Therapeutics for Immune System Health

The incidence of allergic diseases such as atopic dermatitis, allergic rhinitis, allergic asthma and food allergy has increased greatly over the past three decades (especially in developed countries). These conditions are among the most common chronic diseases in the world, affecting approximately 235 million people worldwide according to estimates from the World Health Organization. The increasing incidence of allergic diseases as populations have become urbanized suggests that factors related to a ‘western lifestyle’ are driving this increase. In addition, the use of antibiotics has also been correlated with increased risk of allergic asthma.

A limited number of treatment options exist for atopic allergies. Certain allergy symptoms can be treated with antihistamines, corticosteroid and eicosanoid inhibitors. These approaches only reduce symptoms and do not treat the underlying disease. Also, they may have side effects.

Another approach is allergy immunotherapy. Here the allergen, or a derivative, which causes the allergy, is administered to the patient over a period of time with gradually increasing doses. The purpose is to modify the immunological response to the allergen, resulting in long-term improvement of the patient's immune status. As such, it can be a causal or disease modifying treatment for allergies. Most patients receive at least some symptomatic relief. However, there is the risk that administration of the allergen which induces the allergic reaction could cause IgE mediated adverse events including anaphylactic reactions/shock. Hence, recent attempts have focused on the production of peptide fractions of the allergens which contain one or more epitopes recognized by the T cells involved in the allergic reaction. This peptide approach shows much promise but has yet to be fully evaluated.

Hence, there remains a need for a safe and effective approach for providing patients who suffer from atopic allergies and/or allergic asthma with long term relief; particularly an approach which is disease modifying or effectively addresses causes.

There is now a significant body of evidence supporting that modification of the intestinal microbiota that occurs as a result of westernized lifestyle and antibiotics can disrupt mechanisms that are involved in the development of immune homeostasis and tolerance. For example, long-term deficiency of fiber intake can cause changes in microbiota composition and alter normal immunity, contributing to risk of developing allergic diseases and other inflammatory diseases (Frei et al. 2012). Functionally, allergy results from an inappropriate T-helper type 2 (Th2) immune response (switching to IgE production) against generally innocuous protein. The intestinal microbiota controls systemic Th2 responses by inducing immune homeostasis. Hence, an imbalance (dysbiosis) in the intestinal microbiota can cause impaired immune homeostasis and tolerance, increasing the risk for allergies. It has been demonstrated that SCFA produced by specific groups of microorganisms are key drivers of T-cell subset proliferation and activity (Luu et al. 2020). Bifidobacterium populations in the infant gut has been also related to childhood allergic sensitization (Lynch. 2016).

The incidence of allergic diseases such as atopic dermatitis, allergic rhinitis, allergic asthma and food allergy has increased greatly over the past three decades (especially in developed countries). These conditions are among the most common chronic diseases in the world, affecting approximately 235 million people worldwide according to estimates from the World Health Organization. The increasing incidence of allergic diseases as populations have become urbanized suggests that factors related to a ‘western lifestyle’ are driving this increase. In addition, the use of antibiotics has also been correlated with increased risk of allergic asthma. There is now a significant body of evidence supporting that modification of the intestinal microbiota that occurs as a result of westernized lifestyle and antibiotics can disrupt mechanisms that are involved in the development of immune homeostasis and tolerance. For example, long-term deficiency of fiber intake can cause changes in microbiota composition and alter normal immunity, contributing to risk of developing allergic diseases and other inflammatory diseases (Frei et al. Allergy 67, 451 (2012). Functionally, allergy results from an inappropriate T-helper type 2 (Th2) immune response (switching to IgE production) against generally innocuous protein. The intestinal microbiota controls systemic Th2 responses by inducing immune homeostasis. Hence, an imbalance (dysbiosis) in the intestinal microbiota can cause impaired immune homeostasis and tolerance, increasing the risk for allergies. A limited number of treatment options exist for atopic allergies. Certain allergy symptoms can be treated with antihistamines, corticosteroid and eicosanoid inhibitors. These approaches only reduce symptoms and do not treat the underlying disease. Also, they may have side effects. Another approach is allergy immunotherapy. Here the allergen, or a derivative, which causes the allergy is administered to the patient over a period of time with gradually increasing doses. The purpose is to modify the immunological response to the allergen, resulting in long-term improvement of the patient's immune status. As such, it can be a causal or disease modifying treatment for allergies. Most patients receive at least some symptomatic relief. However, there is the risk that administration of the allergen which induces the allergic reaction could cause IgE mediated adverse events including anaphylactic reactions/shock. Hence recent attempts have focused on the production of peptide fractions of the allergens which contain one or more epitopes recognized by the T cells involved in the allergic reaction. This peptide approach shows much promise but has yet to be fully evaluated. Hence there remains a need for a safe and effective approach for providing patients who suffer from atopic allergies and/or allergic asthma with long term relief; particularly an approach which is disease modifying or effectively addresses causes.

In some aspects, disclosed herein are novel therapeutic strategies for the treatment of diseases or conditions associated with the immune system, such as allergic diseases including atopic dermatitis, allergic rhinitis, allergic asthma and food allergy. In some aspects, the therapeutic method comprises contacting one or more microbes (e.g., bacteria, fungi, yeast) with a composition comprising one or a mixture of oligosaccharides.

In some aspects, disclosed is a method for the treatment of, or for reducing the risk of occurrence of, an atopic allergy, the method comprising administering to a person having the allergy an effective amount of an oligosaccharide selected from one or more of:
(i) an oligosaccharide having a generally linear poly-glucose chain including 1-3 glycosidic linkages,
(ii) an oligosaccharide having a generally linear poly-glucose/mannose chain including interspersed beta 1-4 glucose and beta 1-4 mannose subunits,
(iii) an oligosaccharide a generally linear poly-xylose chain including beta 1-4 glycosidic linkages,
(iv) an oligosaccharide having a generally linear galactose backbone including beta 1-3 glycosidic linkages, and branches from the backbone comprising one or more galactose and/or arabinose including beta 1-6 galactose linkages and/or alpha 1-6 arabinose linkages,
(v) an oligosaccharide having a generally linear poly-mannose backbone including beta 1-4 glycosidic linkages, with single galactose branches including alpha 1-4 glycosidic linkages,
(vi) an oligosaccharide having a generally linear galactose backbone including beta 1-4 glycosidic linkages connected to a pectin fragment,
(vii) an oligosaccharide having a generally linear poly-glucose chain including interspersed beta 1-3 glycosidic and beta 1-4 glycosidic linkages,
(viii) an oligosaccharide having a generally linear poly-glucose chain including beta 1-3 glycosidic and beta 1-4 glycosidic linkages,
(ix) an oligosaccharide having a generally linear poly-glucose backbone including beta 1-4 linkages, and single unit xylose branches linked to the backbone by alpha 1-6 bond,
(x) an oligosaccharide having a generally linear poly-xylose backbone including beta 1-4 glycosidic linkages, and short arabinose branches linked to the galactose through beta 1-2 linkages.

In some aspects, disclosed is a method for reducing the risk of occurrence of an atopic allergy in an infant at risk of developing atopic dermatitis, the method comprising administering to the infant an amount of oligosaccharides, oligosaccharide compositions, or formulations thereof sufficient to modulate T-helper type 2 (Th2) immune response in the infant. The oligosaccharides, oligosaccharide compositions, or formulations thereof may be administered to the infant as sole therapy, in combination with hypoallergenic infant foods, an active vitamin A source and/or an immune-regulating probiotic bacterium, or subsequent to administration of hypoallergenic infant foods, an active vitamin A source and/or an immune-regulating probiotic bacterium.

In some aspects, disclosed is a method for reducing the risk of reoccurrence of an atopic allergy and/or reducing the symptoms of reoccurrence, the method comprising administering to a patient having had an atopic allergy an amount of oligosaccharides, oligosaccharide compositions, or formulations thereof sufficient to modulate T-helper type 2 (Th2) immune response. The oligosaccharides, oligosaccharide compositions, or formulations thereof may be administered to the patent as sole therapy, in combination with hypoallergenic infant foods, an active vitamin A source and/or an immune-regulating probiotic bacterium, or subsequent to administration of hypoallergenic infant foods, an active vitamin A source and/or an immune-regulating probiotic bacterium.

In some aspects, disclosed is a method for reducing allergy symptoms in a patient suffering from an atopic allergy, the method comprising administering to a patient having an atopic allergy an effective amount of oligosaccharides, oligosaccharide compositions, or formulations thereof sufficient and one or more of an immunotherapeutic allergen and/or an active vitamin A source.

In some aspects, disclosed is a method for reducing asthma symptoms, the method comprising administering to a patient having asthma an effective amount of oligosaccharides, oligosaccharide compositions, or formulations thereof. The oligosaccharides, oligosaccharide compositions, or formulations thereof may be administered to the patent as sole therapy, in combination with an active vitamin A source and/or an immune-regulating probiotic bacterium, or subsequent to administration of an active vitamin A source and/or an immune-regulating probiotic bacterium.

In some aspects, the oligosaccharides, oligosaccharide compositions, or formulations thereof are useful as therapeutics in methods for treating, preventing, or improving conditions or disorders associated with immune system health. For example, in some aspects, disclosed herein are methods for the prevention or treatment of allergic diseases by administration of oligosaccharides, oligosaccharide compositions, or formulations thereof.

In some aspects, disclosed herein are novel therapeutic strategies for the treatment of allergic diseases such as atopic dermatitis, allergic rhinitis, allergic asthma and food allergy. In some aspects, the therapeutic method comprises contacting one or more microbes (e.g., bacteria, fungi, yeast) with a composition comprising one or a mixture of CLX101, CLX 102, CLX 103, CLX105, CLX108, CLX109, CLX110, CLX111, CLX112, CLX113, CLX114, CLX115, CLX107, CLX115a, CLX116, CLX117, CLX118, CLX119, CLX122, CLX123, CLX124, CLX124, CLX126, CLX127, CLX128, CLX129, CLX130, CLX131, CLX132, CLX115AL, CLX122DS, CLX122DSF and CLX133 to (i) selectively stimulate growth of one or more microbes and/or (ii) increase the production of beneficial metabolites such as SCFA which regulates T-cell activities in the lamina propria. In some aspects, the microbe stimulated by the oligosaccharide(s) described herein is a microbe in the gut, comprising at least one of Lactobacillus crispatus, Lactobacillus rhamnosus, Bifidobacteria, Bifidobacterium pseudocatenulatum, Bifidobacterium longum subsp. longum, Bifidobacterium animalis, Firmicutes, Clostridium butyricum, Ruminococcus, Ruminococcus gnavus, Blautia, Roseburia, or any combination thereof. One or more of CLX101, CLX 102, CLX 103, CLX105, CLX108, CLX109, CLX110, CLX111, CLX112, CLX113, CLX114, CLX115, CLX107, CLX115a, CLX116, CLX117, CLX118, CLX119, CLX122, CLX123, CLX124, CLX124, CLX126, CLX127, CLX128, CLX129, CLX130, CLX131, CLX132, CLX115AL, CLX122DS, CLX122DSF and CLX133 can be combined with other ingredients to produce foodstuffs and supplements including infant formula, geriatric supplements, drinks, nutritional supplements, baking flours, and snack foods. One or more of these oligosaccharides can also be used as pharmaceutical products.

Thus, in some aspects, disclosed is the administration of a pharmaceutically acceptable composition of one or more CLX101, CLX 102, CLX 103, CLX105, CLX108, CLX109, CLX110, CLX111, CLX112, CLX113, CLX114, CLX115, CLX107, CLX115a, CLX116, CLX117, CLX118, CLX119, CLX122, CLX123, CLX124, CLX124, CLX126, CLX127, CLX128, CLX129, CLX130, CLX131, CLX132, CLX115AL, CLX122DS, CLX122DSF and CLX133 to stimulate the growth of Bifidobacteria. In some aspects, disclosed is the administration of a pharmaceutically acceptable composition of one or more CLX101, CLX 102, CLX 103, CLX105, CLX108, CLX109, CLX110, CLX111, CLX112, CLX113, CLX114, CLX115, CLX107, CLX115a, CLX116, CLX117, CLX118, CLX119, CLX122, CLX123, CLX124, CLX124, CLX126, CLX127, CLX128, CLX129, CLX130, CLX131, CLX132, CLX115AL, CLX122DS, CLX122DSF and CLX133 to increase the production of SCFA. Pharmaceutically acceptable salts, stereoisomers, and metabolites of one or more of the oligosaccharides described herein also are contemplated.

In some aspects, administration of and administering a compound or composition should be understood to mean providing a compound or salt thereof or a pharmaceutical composition comprising a compound. The compound or composition can be administered by another person to the patient (e.g., intravenously) or it can be self-administered by the subject (e.g., tablets or capsules). The term “patient” refers to mammals (for example, humans and veterinary animals such as dogs, cats, pigs, horses, sheep, and cattle). Administration of one or more of CLX101, CLX 102, CLX 103, CLX105, CLX108, CLX109, CLX110, CLX111, CLX112, CLX113, CLX114, CLX115, CLX107, CLX115a, CLX116, CLX117, CLX118, CLX119, CLX122, CLX123, CLX124, CLX124, CLX126, CLX127, CLX128, CLX129, CLX130, CLX131, CLX132, CLX115AL, CLX122DS, CLX122DSF and CLX133 in combination with other compounds, such as excipients, colorants, fillers, binders, diluents, buffering agents, moistening agents, preservatives, flavoring agents, dyes, disintegrating agents, and pharmaceutically compatible carriers, is contemplated.

In some aspects, the bioactive oligosaccharides can modulate the immune system (e.g., to under or overreact to known and unknown stimuli).

### Mechanistic Relationship Between Modulation and Therapeutics

Modulation of disease through the microbiome is an attractive approach for many diseases. The microbiome interacts with the host metabolism and immune system through numerous metabolites that act as metabolic precursors, pH modulators, and/or signaling molecules. One such strategy for modulating the microbiome and its metabolism is by the application of a prebiotic. Prebiotics are carbohydrates containing compounds that escape digestion by host enzymes and make it to the intestine where they are digested by microorganisms in the gut. Appropriate prebiotics should have structures (monosaccharides, glycosidic linkages, branches, and sizes) that allow consumption only by a preferred set of bacteria in the gut that through their own enzymatic capacity or through cross feeding can consume the carbohydrates and increase their absolute abundances, relative abundances, and/or metabolic capabilities in such a way that they can produce enough of a given metabolite or group of metabolites to induce the desired health effect.

The mechanisms illustrated below provide a logical and scientific connection between (a) various health indications and (b) modulation of microbiota and/or their metabolic outputs, thereby demonstrating that the oligosaccharides, oligosaccharide compositions, and/or formulations thereof disclosed herein can be used as therapeutics for gastrointestinal health, cardiovascular health, renal system health, central nervous system (CNS) health, and/or immune system health.

### Mechanism 1—In Vitro Culture Model to Test the Effect of Oligosaccharides on Gut Inflammation

This mechanism pertains at least to gut health, gut-brain axis, and metabolic and cardiovascular related diseases, conditions, disorders, and/or indications.

One such mechanism pertains to the production of enough of a given metabolite or group of metabolites to induce mucosal immune responses (Marsland et al. 2016). Specific microbial metabolites like acetate, propionate, butyrate, beta-hydroxybutyrate, valeric acid, lithocholic acid, deoxycholic acid, metabolites, indole, indole-3-aldehyde, indole lactic acid, indole propionate, and/or tryptamine, among others, have been described as key players in normal immune development and progression of inflammation in diseases like gut health, gut-brain axis related disorder, metabolic and/or cardiovascular diseases (Lavelle et al. 2020, Obrenovich et al. 2016, Witkowski et al. 2020, Zietek et al. 2016). Therefore, an increase in the relative abundance of microorganisms with the ability to produce those metabolites in the gut may reduce, influence, prevent, and/or treat gut inflammation.

Epithelial cell lines from the intestine (human enterocyte-like Caco-2 cells) are used in in vitro culture models, to measure the host response elicited by gut bacterial populations grown in the presence of oligosaccharides. To stimulate inflammation of the epithelial lining, epithelial cells are co-cultured with THP-1 cells. The inflamed epithelial cells are then challenged with fecal fermentation supernatants, which are derived from filtration of fecal samples cultured with oligosaccharides. Supernatants were generated as described in Example 13 with each of oligosaccharides CLX101, CLX 102, CLX 103, CLX105, CLX108, CLX109, CLX110, CLX111, CLX112, CLX113, CLX114, CLX115, CLX107, CLX115a, CLX116, CLX117, CLX118, CLX119, CLX122, CLX123, CLX124, CLX124, CLX126, CLX127, CLX128, CLX129, CLX130, CLX131, CLX132, CLX115AL, CLX122DS, CLX122DSF and CLX133.

Human enterocyte-like Caco-2 cells are cultured on transwell inserts (0.4 um pore size) in 24-well tissue culture plates for 18-22 days at 37 C/5% CO2 in DMEM (Amimed) supplemented with 10% heat-inactivated fetal calf serum and 0.1% penicillin/streptomycin. The cell culture media is changed every second day until the cells are fully differentiated. Simultaneously, PMA-differentiated THP-1 cells are cultured in separate 24 well plates for 4 days 37 C/5% CO2 in the aforementioned culture media. The cell culture media is changed every second day. The Caco-2 seeded transwell inserts are then added to the PMA-differentiated THP-1 cells. To induce inflammation, the THP-1 cells are stimulated with lipopolysaccharide (LPS) and interferon-gamma (IFN-gamma) and the cells are co-incubated for 48 hours prior to being challenged with fecal fermentation supernatants. The cells are incubated for 24-48 hours with the fecal fermentation supernatants. After incubation, the cell supernatants are collected and analyzed for inflammatory (TNF-α, IL1-β and IL-6) and anti-inflammatory markers (IL-10, TGF-beta, IL-4). This analysis is performed by enzyme linked immunosorbent assay (ELISA) or Q-PCR following standard protocols.

Analysis of ELISA and Q-PCR results shows a decrease of the inflammatory markers TNF-α, IL1-β and IL-6 and an increase of IL-10, TGF-beta, IL-4 (anti-inflammatory). This data indicates that oligosaccharides CLX101, CLX102, CLX103, CLX108, CLX109, CLX110, CLX111, CLX112, CLX113, XLX114, CLX115, CLX107, CLX115a, CLX117, CLX122, CLX115AL, CLX122DS, CLX122DSF, CLX123, CLX125, CLX126, CLX127, CLX128, and CLX131 generate changes in the microbial community and production of bacterial metabolites that can decrease inflammation in the gut epithelial cells, with potential application in gut health, gut-brain axis and metabolic and cardiovascular diseases. Oligosaccharides having similar structural and/or physical features (as disclosed herein) are expected for perform similarly.

References for this mechanism are below, each of which is incorporated by reference in its entirety for all purposes:
Marsland. Nature Medicine 22 (2016) Regulating inflammation with microbial metabolites
Lavelle et al. Gut microbiota-derived metabolites as key actors in inflammatory bowel disease. Nature Reviews Gastroenterology & Hepatology (2020), 17:223.
Obrenovich et al. The co-metabolism within the gut-brain metabolic interaction: Potential targets for drug treatment and design. CNS Neurol Disord Drug Targets (2016) 15:127 Witkowski et al. Gut microbiota and cardiovascular disease. Circulation research (2020) 127:553
Zietec et al. Inflammation meets metabolic diseases: Gut feeling mediated by GLP-1. Front Immunol (2016) 7:154

### Mechanism 2—In Vitro Culture Model to Test the Effect of Oligosaccharides on GLP-1 Production

This mechanism pertains at least to gut health, gut-brain axis, and metabolic and cardiovascular related diseases, conditions, disorders, and/or indications.

One such mechanism pertains to the production of enough of a given metabolite or group of metabolites to induce mucosal immune responses (Marsland et al. 2016). Specific microbial metabolites like acetate, propionate, butyrate, lithocholic acid, deoxycholic acid, among others, can trigger the production of GLP-1 by L-cells by different mechanisms that involve receptors like GPR41, GPR43, GPR109A and/or TGR5 (Park et al. 2021, Duboc et al. 2014). An increase in the relative abundance of microorganisms with the ability to produce molecules that act as agonist to any of these receptors and increase production of GLP-1 is considered a therapeutic target for progression of inflammation in diseases like gut health, gut-brain axis related disorder, metabolic and/or cardiovascular diseases (Everard et al. 2014; Greiner et al. 2016). Therefore, an increase in the relative abundance of microorganisms with the ability to produce molecules that act as agonists to GLP-1 related receptors may reduce, influence, prevent, and/or treat gut inflammation such indications.

To assess the ability of oligosaccharides to stimulate production of metabolites that affect GLP-1 secretion, we use GLUTag and/or NCI-H716 L-cell lines. Both types of cell lines are cultured in DMEM (Amimed). Cell lines are incubated with supernatants from oligosaccharide-supplemented fecal fermentations for 1-2h. Supernatants were generated as described in Example 13 with each of oligosaccharides CLX101, CLX 102, CLX 103, CLX105, CLX108, CLX109, CLX110, CLX111, CLX112, CLX113, CLX114, CLX115, CLX107, CLX115a, CLX116, CLX117, CLX118, CLX119, CLX122, CLX123, CLX124, CLX124, CLX126, CLX127, CLX128, CLX129, CLX130, CLX131, CLX132, CLX115AL, CLX122DS, CLX122DSF and CLX133.

Supernatants from treated cell lines are collected, centrifuged and then assessed for active GLP-1 using an enzyme-linked immunosorbent assay kit (Merck-Millipore), normalized to total proteins.

Analysis of GLP-1 levels shows an increase in this hormone with supernatants from the fecal fermentations, which contain metabolites that ignite the expression of this hormone. Because of the impact of an increase of GLP-1 in the host, results indicate that the oligosaccharides CLX101, CLX102, CLX108, CLX109, CLX110, CLX111, CLX112, CLX113, CLX114, CLX115, CLX122, CLX122DS, CLX122DSF, CLX125, and CLX128 and those having similar or substantially similar (as disclosed herein) structural and/or physical characteristics could be used to modulate gut health, gut-brain axis related diseases and metabolic and cardiovascular diseases.

References for this mechanism are below, each of which is incorporated by reference in its entirety for all purposes:
Park et al. Bidirectional regulatory potentials of short-chain fatty acids and their G-protein-coupled receptors in autoimmune neuroinflammation. Scientific Reports (2021) 9: 8837
Duvok et al. The bile acid TGR5 membrane receptor: From basic research to clinic development. Digestive and Liver Disease (2014) 46: 302
Everard et al. Gut microbiota and GLP-1. Rev Endocr Metab Disord (2014) 15:189
Greiner et al. Microbial regulation of GLP-1 and L-cell biology. Molecular metabolism (2016) 5:753

### Mechanism 3—In Vitro Culture Model to Test the Effect of Oligosaccharides on Histone Deacetylases Inhibition

This mechanism pertains at least to gut health, gut-brain axis, and metabolic and cardiovascular related diseases, conditions, disorders, and/or indications.

One such mechanism pertains to the production of enough of a given metabolite or group of metabolites to inhibit histone deacetylases (HDAC). HDAC class I inhibitors play a role in the inhibition of inflammation and immunity (Yuille et al. 2018). Specific microbial metabolites like butyrate, beta-hydroxybutyrate and/or valeric acid, among others, can inhibit class I HDAC (Yuille et al. 2018; Chriett et al. 2019). Therefore, an increase in the relative abundance of microorganisms with the ability to produce molecules that act as inhibitors of histone deacetylase (HDAC) has been described as a therapeutic target for gut health, gut-brain axis related diseases and metabolic and cardiovascular diseases (Yuille et al. 2018, Stilling et al. 2016, Yoon et al. 2016).

To assess the ability of oligosaccharides to inhibit HDAC, human colorectal adenocarcinoma HT29 cell lines are cultured in DMEM (Amimed). Cell lines are incubated with supernatants from oligosaccharide-supplemented fecal fermentations for 48 h prior to nuclear protein extraction. Supernatants were generated as described in Example 13 with each of oligosaccharides CLX101, CLX 102, CLX 103, CLX105, CLX108, CLX109, CLX110, CLX111, CLX112, CLX113, CLX114, CLX115, CLX107, CLX115a, CLX116, CLX117, CLX118, CLX119, CLX122, CLX123, CLX124, CLX124, CLX126, CLX127, CLX128, CLX129, CLX130, CLX131, CLX132, CLX115AL, CLX122DS, CLX122DSF and CLX133. For nuclear protein extraction cells are centrifuged and NXTRACT NuCLEAR kit (Sigma Aldrich) is used following manufacturer/s instruction. Once extracted, the nuclear proteins are snap-frozen. Class I HDACs activity is analyzed using a fluorogenic assay kit (BPS Bioscience).

Analysis of fluorescence results shows that supernatants from fecal fermentations performed with the oligosaccharides CLX101, CLX102, CLX108, CLX109, CLX110, CLX111, CLX112, CLX113, CLX114, CLX115, CLX115AL, CLX122, CLX122DS, CLX122DSF, CLX126, and CLX128 inhibit the activity of HDACs. Because of the impact of inhibition of this group of enzymes in the host, the oligosaccharides CLX101, CLX102, CLX108, CLX109, CLX110, CLX111, CLX112, CLX113, CLX114, CLX115, CLX115AL CLX122, CLX122DS, CLX122DSF, CLX126, and CLX128 and those having similar or substantially similar (as disclosed herein) structural and/or physical characteristics could be used to modulate gut health, gut-brain axis related diseases and metabolic and cardiovascular diseases.

References for this mechanism are below, each of which is incorporated by reference in its entirety for all purposes:
Yullie et al. Human gut bacteria as potent class I histone deacetylase inhibitors in vitro through production of butyric acid and valeric acid. PLOS One (2018)
Chriett et al. Prominent action of butyrate over beta-hydroxybutyrate as histone deacetylase inhibitor, transcriptional modulator and anti-inflammatory molecule. Scientific Reports (2019) 9:742
Chang et al. The microbial metabolite butyrate regulates intestinal macrophage function via histone deacetylase inhibition. PNAS (2014), 11: 2247.
Stilling et al. The neuropharmacology of butyrate: The bread and butter of the microbiota-gut-brain axis. Neurochemistry International (2016) 99:1
Yoon et al. HDAC and HDAC inhibitor: From Cancer to Cardiovascular Diseases. Chonnam Med J (2016) 52:1

### Mechanism 4—In Vitro Culture Model to Test the Effect of Oligosaccharides on Ary Hydrocarbon Receptor Activation.

This mechanism pertains at least to gut health, gut-brain axis, and metabolic and cardiovascular related diseases, conditions, disorders, and/or indications.

One such mechanism pertains to the production of enough of a given metabolite or group of metabolites that acts as agonist to the Ary hydrocarbon receptor (AhR). Specific microbial metabolites like butyrate, indole, indole-3-aldehyde, indole lactic acid, indole propionate, and/or tryptamine among others, have been described as AhR agonists (Lamas et al. 2020, Agus et al. 2018). AhR agonists are key in the regulation of many chronic inflammatory conditions that affect not only general gut health, but also gut-brain axis, metabolic and cardiovascular disease (Neavin et al. 2018, Barroso et al. 2021, Zhang et al. 2011).

To assess the ability of oligosaccharides to activate AhR, human epithelial cell lines HT29-AhR and Caco2-AhR reporter cell lines are produced by electroporation using pGL4.43 [luc2P/XRE/Hygro] (Promega) and the Nucleofector device (Lonza) according to manufacturer's recommendations. Cell lines are incubated with supernatants from oligosaccharide-supplemented fecal fermentations during 24 hours. Supernatants were generated as described in Example 13 with each of oligosaccharides CLX101, CLX 102, CLX 103, CLX105, CLX108, CLX109, CLX110, CLX111, CLX112, CLX113, CLX114, CLX115, CLX107, CLX115a, CLX116, CLX117, CLX118, CLX119, CLX122, CLX123, CLX124, CLX124, CLX126, CLX127, CLX128, CLX129, CLX130, CLX131, CLX132, CLX115AL, CLX122DS, CLX122DSF and CLX133. Luciferase activity is measured as relative luminescence units, using a microplate reader (BioTek) and the Neolite Luciferase Assay System.

Analysis of fluorescence results shows that supernatants from fecal fermentations performed with the oligosaccharides CLX101, CLX102, CLX108, CLX109, CLX110, CLX111, CLX112, CLX113, CLX114, CLX115, CLX115AL, CLX122, CLX122DS, CLX122DSF CLX126, CLX128, and CLX131 increase activity of AhR receptor. Because of the impact of the activation of this receptor in the host, the oligosaccharides CLX101, CLX102, CLX108, CLX109, CLX110, CLX111, CLX112, CLX113, CLX114, CLX115, CLX115AL, CLX122, CLX126, CLX128, and CLX131 and those having similar or substantially similar (as disclosed herein) structural and/or physical characteristics could be used to modulate gut health, gut-brain axis related diseases and metabolic and cardiovascular diseases.

References for this mechanism are below, each of which is incorporated by reference in its entirety for all purposes:
Neavin et al. The role of the Aryl Hydrocarbon Receptor (AhR) in immune and inflammatory diseases. Int J Mol Sci (2018) 19:3851
Barroso et al. The aryl hydrocarbon receptor and the gut-brain axis. Cellular and Molecular Immunology (2021) 18:259
Zhang et al. The role of endogenous aryl hydrocarbon receptor signaling in cardiovascular physiology. J. Cardiovasc Dis Res (2011) 2:91
Lamas et al. Aryl hydrocarbon receptor ligand production by the gut microbiota is decreased in celiac disease leading to intestinal inflammation. Sci Transl Med (2020), 12:566
Agus et al. Gut Microbiota Regulation of Tryptophan metabolism in Health and Disease. Cell Host and Microbes (2018) 23:716.

### Mechanism 5—In Vitro Culture Model to Test the Effect of Oligosaccharides on Pregnane X Receptor (PXR) Activation.

This mechanism pertains at least to gut health, gut-brain axis, and metabolic and cardiovascular related diseases, conditions, disorders, and/or indications.

One such mechanism pertains to the production of enough of a given metabolite or group of metabolites that acts as agonist to the PXR receptor (Dovrak et al. 2020). Specific microbial metabolites like propionate, indole, indole-3 acetamine, lithocholic acid and 3-keto lithocholic acid have been described as agonists of PXR (Li et al. 2021, Xie et al. 2001). PXR is essential in maintaining intestinal homeostasis, abrogating inflammation. By decreasing inflammation, activation of PXR has a beneficial impact in gut health, gut-brain axis, metabolic and cardiovascular diseases (Venkatesh et al.2014, Bosi et al.2020, Daujat-Chavanieu et al. 2020).

To assess the ability of oligosaccharides to activate PXR, human epithelial cell lines LS180 are transiently transfected with human PXR expression vector pSG5-hPXR and p3A4-Luc reporter construct by lipofection (FuGENE® HD Transfection reagent), according to manufacturer's recommendations. Cell lines are incubated with supernatants from oligosaccharide-supplemented fecal fermentations during 24 hours. Supernatants were generated as described in Example 13 with each of oligosaccharides CLX101, CLX 102, CLX 103, CLX105, CLX108, CLX109, CLX110, CLX111, CLX112, CLX113, CLX114, CLX115, CLX107, CLX115a, CLX116, CLX117, CLX118, CLX119, CLX122, CLX123, CLX124, CLX124, CLX126, CLX127, CLX128, CLX129, CLX130, CLX131, CLX132, CLX115AL, CLX122DS, CLX122DSF and CLX133. Following cell lysis, luciferase activity is measured as relative luminescence units, using a microplate reader (BioTek) and the Neolite Luciferase Assay System.

Analysis of fluorescence results shows that supernatants from fecal fermentations performed with the oligosaccharides CLX 103, CLX109, CLX111, CLX114, CLX107, CLX115a, CLX117, CLX123, CLX125, CLX126, CLX127, CLX128, and CLX131 increase the activity of the PXR receptor. Because of the impact of the activation of this receptor in the host, the oligosaccharides CLX 103, CLX109, CLX111, CLX114, CLX107, CLX115a, CLX117, CLX123, CLX125, CLX126, CLX127, CLX128, and CLX131 and those having similar or substantially similar (as disclosed herein) structural and/or physical characteristics could be used to modulate gut health, gut-brain axis related diseases and metabolic and cardiovascular diseases.

References for this mechanism are below, each of which is incorporated by reference in its entirety for all purposes:
Drovak et al. Targeting the pregnane X receptor using microbial metabolite mimicry. EMBO Molecular Medicine (2020) 12
Li et al. New insights into gut-bacteria derived indole and its derivatives in intestinal and liver diseases. Front Pharmacol (2021) 13
Xi et at. An essential role for nuclear receptors SXR/PXR in detoxification of cholestatic bile acids. PNAS (2001) 89
Venkatesh et al. Symbiotic bacterial metabolites regulate gastrointestinal barrier function via the xenobiotic sensor PXR and Toll-like Receptor 4. Immunity (2014) 41
Daujat-Chavanieu et al. Regulation of CAR and PXR expression in health and disease. Cells (2020) 9:2395

### Mechanism 6—In Vitro Culture Model to Evaluate Effect of Oligosaccharides in the Permeability of Gut Epithelial Cells

This mechanism pertains at least to gut health, gut-brain axis, and metabolic and cardiovascular related diseases, conditions, disorders, and/or indications.

One such mechanism pertains to the production of enough of a given metabolite or group of metabolites that influences barrier integrity. Short chain fatty acids, tryptophan metabolites, lithocholic acid and/or 3-keto lithocholic acid, among other microbial metabolites, have been linked to gut epithelial cells restoration (Chakoroun et al. 2020, Usuda et al. 2021, Raimondi et al. 2008). Restoring damaged epithelial cells beneficially impacts gut health, gut-brain axis, metabolic and cardiovascular diseases (Sommer et al. 2021; Sgambato et al. 2016; Zhong et al. 2016).

To assess the ability of oligosaccharides to affect permeability cell lines, human enterocyte-like Caco-2 cells are seeded at a density of 1.5×10\hat{ }4 cells/insert on 13 mm cell culture inserts. The inserts are placed into 24-well tissue culture plates and cultured for 21 days in DMEM (Amimed). The cell culture media is changed every second day until cells are fully differentiated. Cell lines are incubated with supernatants from oligosaccharide-supplemented fecal fermentations during 24 hours. Supernatants were generated as described in Example 13 with each of oligosaccharides CLX101, CLX 102, CLX 103, CLX105, CLX108, CLX109, CLX110, CLX111, CLX112, CLX113, CLX114, CLX115, CLX107, CLX115a, CLX116, CLX117, CLX118, CLX119, CLX122, CLX123, CLX124, CLX124, CLX126, CLX127, CLX128, CLX129, CLX130, CLX131, CLX132, CLX115AL, CLX122DS, CLX122DSF and CLX133. At the conclusion of the incubation period, transepithelial electrical resistance (TEER) is determined using a Millicell-ERS2 voltmeter/ohmmeter.

In addition, to evaluate the effect of oligosaccharides on barrier function is to determine their effect in increasing the expression of gut epithelial mucin and tight junction related genes. For this goal, co-culture of human enterocyte-like CaCo2 cells and goblet cells (HT-29) are used.

Caco-2 and HT-29 cells are grown separately in tissue culture flasks in DMEM. Monocultures of Caco-2 and HT-29 cells are seeded on the apical chamber of 13 mm cell culture inserts at a proportion of 9:1 with a final concentration of 1.5×10\hat{ }4 cells/insert. The inserts are placed into 24-well tissue culture plates and cultured for 21 days in DMEM (Amimed). The cell culture media is changed every second day until cells are fully differentiated. To assess the ability of oligosaccharides to affect expression of mucin production and tight junctions related genes, cell lines are incubated with supernatants from oligosaccharide-supplemented fecal fermentations during 24 hours. Supernatants were generated as described in Example 13 with each of oligosaccharides CLX101, CLX 102, CLX 103, CLX105, CLX108, CLX109, CLX110, CLX111, CLX112, CLX113, CLX114, CLX115, CLX115AL, CLX107, CLX115a, CLX116, CLX117, CLX118, CLX119, CLX122, CLX122DS, CLX122DSF, CLX123, CLX124, CLX124, CLX126, CLX127, CLX128, CLX129, CLX130, XLX131, CLX132, and CLX133. After incubation, expression of Muc2 genes and tight junction related genes (occludin, claudin-4, ZO-1) are measured by Q-PCR.

Analysis of TEER results in the Caco2 cell model show a decrease in permeability when cells are incubated in the presence of fecal supernatants. Analysis of Q-PCR results shows an increase in the expression of Muc2, occludin, claudin-4 and ZO-1 genes when cells are incubated in the presence of fecal supernatants. Both results suggest that the oligosaccharides CLX101, CLX102, CLX103, CLX108, CLX109, CLX110, CLX111, CLX112, CLX113, CLX114, CLX115, CLX115AL, CLX107, CLX115a, CLX117, CLX122, CLX122DS, CLX122DSF, CLX123, CLX125, CLX126, CLX127, CLX128, and CLX131 generate change in microbial community profile and production of bacterial metabolites, impacting gut permeability and thus, with potential application in gut health, gut-brain axis and metabolic and cardiovascular diseases. Oligosaccharides having similar or substantially similar (as disclosed herein) structural and/or physical characteristics are expected to perform similarly.

References for this mechanism are below, each of which is incorporated by reference in its entirety for all purposes:
Sommer et al. Intestinal Mucosal Wound healing and barrier integrity in IBD-crosstalk and trafficking of cellular players. Front Med (2021) 8
Sgambato et al. Gut-brain axis in Gastric Mucosal Damage and protection. Curr Neuropharmacol (2016) 14:959
Zhong et al. Type 2 diabetes mellitus is associated with more serious small intestinal mucosal injuries. PLOs One (2016)
Chakaroun et al. Gut Microbiome, intestinal permeability, and tissue bacteria in metabolic disease: Perpetrators or bystanders?. Nutrients (2020) 12: 1082
Usuda et al. Leaky gut: Effect of Dietary fiber and fats on microbiome and intestinal barrier. Int J Mol Sci (2021) 22:7613
Raimondi et al. Bile acids modulate tight junction structure and barrier function of Caco-2 monolayers via EGFR activation. Am J Physiol Gastrointest Liver Physiol (2008) 294: G906.

### Mechanism 7—In Vivo Model to Test the Effect of Oligosaccharides in Chemically Induced Intestinal Inflammation

This mechanism pertains at least to gut health related diseases, conditions, disorders, and/or indications.

DSS-induced colitis mouse model is a common model for induced intestinal inflammation and colitis, being robust, reproducible and expresses an overall etiology, including immunological and histological changes in the GI tract, that resembles human disease.

C57BL/6 mice (6 weeks old) are used to evaluate the therapeutic effect of oligosaccharides during dextran sulfate sodium (DSS)-induced colitis. Mice are divided in 30 groups (n=10) and fed with a standard diet during 14 days. During this period, all the groups of mice but the control group receive oligosaccharide in the drinking water (10% w/v). The intervention oligosaccharides are: CLX101, CLX 102, CLX 103, CLX105, CLX108, CLX109, CLX110, CLX111, CLX112, CLX113, CLX114, CLX115, CLX107, CLX115a, CLX116, CLX117, CLX118, CLX119, CLX122, CLX123, CLX124, CLX124, CLX126, CLX127, CLX128, CLX129, CLX130, CLX131, CLX132, CLX115AL, CLX122DS, CLX122DSF and CLX133. After 14 days of diet-treatment, animals are further treated with 2.5% dextran sulfate sodium (DSS) in water for 7 additional days. The animals are euthanized and intestinal tissue sampling is performed. Fecal pellets are collected on day 0, day 7, 14 and day 21 of supplementation, and bacterial DNA diversity is assessed by 16S rRNA sequencing. Microbial communities were profiled by sequencing the V4 region of the bacterial 16s rRNA gene amplified using 515F (5′-GTGCCAGCMGCCGCGGTAA-3′) and 806R (5′-GGACTACHVGGGTWTCTAA-3′) primers. NovaSeq 6000 was used to obtain 250 bp paired end reads.

Fecal pellets are collected on day 0, day 14, and day 28 of supplementation, and bacterial DNA diversity is assessed by 16S rRNA sequencing. Microbial communities were profiled by sequencing the V4 region of the bacterial 16s rRNA gene amplified using 515F (5′-GTGCCAGCMGCCGCGGTAA-3′) and 806R (5′-GGACTACHVGGGTWTCTAA-3′) primers. NovaSeq 6000 was used to obtain 250 bp paired end reads.

The consistency of feces is assessed in a blinded manner using the diarrhea score (Sakai et al. 2013): 0: normal, dry and solid feces; 1: swollen, moist feces; 2: soft and mucous feces; 3: wet and shapeless feces; 4: bloody diarrhea.

Ninety minutes prior to euthanasia, the mice are intragastrically gavaged with 200 μL of a 60 mg/ml FITC-Dextran (Sigma) solution in double-distilled water. FITC-dextran is a non-metabolizable molecule that is used to assess intestinal permeability, with higher serum FITC-dextran concentrations indicating more efficient translocation of the molecule past the intestinal barrier and higher intestinal permeability. Blood serum is collected after centrifugation at 1500×g for 15 min. Serum fluorescence intensity is measured using a multi-detection microplate reader (Tecan Infinite® M200 Pro) FITC concentration is calculated from a standard curve using serial dilutions of FITC-dextran.

The mice are euthanized using CO 2.

Mouse cecal contents are isolated and centrifuged at 4° C. and 8,000×g for 10 min. Short Chain Fatty Acid analysis is done by HPLC using a cation-H refill cartridge (30×4.6 mm) and an Aminex HPX-87H column at a flow rate of 0.4 ml.min-1 for 60 minute and eluted with 10 mM sulphuric acid solution.

Quantification is done by detection of refractive index. The concentration is calculated by integral area comparison with authentic standard solutions.

A tissue sample of 2 cm from distal colon is isolated, briefly washed with PBS, fixed in 10% neutral buffered formalin (Sigma) for at least 24 h and processed for paraffin embedding and sectioning. Histopathological analysis to determine colitis scores is performed on deparaffinized 5 m Hematoxylin and Eosin (Sigma) stained tissue sections. Sections are scored individually by an independent investigator blinded to the type of treatment.

Tissue samples are also analyzed for inflammatory markers, cytokines, and markers of epithelial integrity. Tissue from proximal or distal colon is homogenized with a Precellys 24 tissue homogenizer (Bertinlnstruments) and RNA is isolated using Trizol reagent (Sigma) following manufacturer's instructions. Total RNA (1 ul) is reverse transcribed. Q-PCR is used to measure cytokine expression (IL-1beta, IL-6, TGFbeta, TNF-alpha), as well as expression of tight junction proteins occludin and claudin-1. Lipocalin-2 levels, a marker for murine gut inflammation, is measured in fecal samples by ELISA (Invitrogen). Myeloperoxidase (MPO) is a proinflammatory enzyme stored in the azurophilic granules of neutrophil granulocytes, and an indicator of inflammation. Neutrophil recruitment and accumulation is determined by measuring enzymatic activity of MPO.

Large intestine is collected for immunological analysis by flow cytometry. T cells response is measured in both groups of mice, by the analysis of T cell populations in small intestine lamina propria using flow cytometry.

Analysis of inflammatory markers, cytokines, and markers of epithelial integrity in the distal colon of mice after 3 week of supplementation indicate that the mice receiving the intervention oligosaccharides experience decreased gut inflammation, and thus, with potential application in gut health. Oligosaccharides having similar or substantially similar (as disclosed herein) structural and/or physical characteristics are expected to perform similarly.

### Mechanism 8—In Vivo Model to Test the Effect of Oligosaccharides in Spontaneous Intestinal Inflammation

This mechanism pertains at least to gut health related diseases, conditions, disorders, and/or indications.

Mice deficient in IL-104 or the IL-10 receptor 5 develop spontaneous colitis early in life and are one of the most widely used animal models for studying the pathogenesis of chronic inflammation like human IBD.

IL10 −/− mice (3 weeks old) are used to evaluate the effect of oligosaccharides in spontaneous gut inflammation. The IL-10 knockout model is selected because the lack of the immunosuppressive effect mediated by interleukin-10 leads to a progressive enterocolitis (Kuhn et al., 1993) and is a model of ulcerative colitis. Mice are divided in 30 groups (n=10) and fed with a standard diet during 28 days. During this period, all the groups of mice but the control group receive oligosaccharide in the drinking water (10% w/v). The intervention oligosaccharides are: CLX101, CLX 102, CLX 103, CLX105, CLX108, CLX109, CLX110, CLX111, CLX112, CLX113, CLX114, CLX115, CLX107, CLX115a, CLX116, CLX117, CLX118, CLX119, CLX122, CLX123, CLX124, CLX124, CLX126, CLX127, CLX128, CLX129, CLX130, CLX131, CLX132, CLX115AL, CLX122DS, CLX122DSF and CLX133. The animals are euthanized and intestinal tissue sampling is performed. Fecal samples are collected during and at the end of the experiment.

Fecal pellets are collected on day 0, day 14, and day 28 of supplementation, and bacterial DNA diversity is assessed by 16S rRNA sequencing. Microbial communities were profiled by sequencing the V4 region of the bacterial 16s rRNA gene amplified using 515F (5′-GTGCCAGCMGCCGCGGTAA-3′) and 806R (5′-GGACTACHVGGGTWTCTAA-3′) primers. NovaSeq 6000 was used to obtain 250 bp paired end reads.

The consistency of feces is assessed in a blinded manner using the diarrhea score (Sakai et al. 2013): 0: normal, dry and solid feces; 1: swollen, moist feces; 2: soft and mucous feces; 3: wet and shapeless feces; 4: bloody diarrhea.

Ninety minutes prior to euthanasia, the mice are intragastrically gavaged with 200 μL of a 60 mg/ml FITC-Dextran (Sigma) solution in double-distilled water. FITC-dextran is a non-metabolizable molecule that is used to assess intestinal permeability, with higher serum FITC-dextran concentrations indicating more efficient translocation of the molecule past the intestinal barrier and higher intestinal permeability. Blood serum is collected after centrifugation at 1500×g for 15 min. Serum fluorescence intensity is measured using a multi-detection microplate reader (Tecan Infinite® M200 Pro) FITC concentration is calculated from a standard curve using serial dilutions of FITC-dextran.

The mice are euthanized using CO 2.

Mouse cecal contents are isolated and centrifuged at 4° C. and 8,000×g for 10 min. Short Chain Fatty acids were analyzed as in the method described in Example 13.

A tissue sample of 2 cm from distal colon is isolated, briefly washed with PBS, fixed in 10% neutral buffered formalin (Sigma) for at least 24 h and processed for paraffin embedding and sectioning. Histopathological analysis to determine colitis scores is performed on deparaffinized 5 m Hematoxylin and Eosin (Sigma) stained tissue sections. Sections are scored individually by an independent investigator blinded to the type of treatment.

Tissue samples are also analyzed for inflammatory markers, cytokines, and markers of epithelial integrity. Tissue from proximal or distal colon is homogenized with a Precellys 24 tissue homogenizer (Bertinlnstruments) and RNA is isolated using Trizol reagent (Sigma) following manufacturer's instructions. Total RNA (1 ul) is reverse transcribed. Q-PCR is used to measure cytokine expression (IL-1beta, IL-6, TGFbeta, TNF-alpha), as well as expression of tight junction proteins occludin and claudin-1. Lipocalin-2 levels, a marker for murine gut inflammation, is measured in fecal samples by ELISA (Invitrogen). Myeloperoxidase (MPO) is a proinflammatory enzyme stored in the azurophilic granules of neutrophil granulocytes, and an indicator of inflammation. Neutrophil recruitment and accumulation is determined by measuring enzymatic activity of MPO.

Large intestine is collected for immunological analysis by flow cytometry. T cells response is measured in both groups of mice, by the analysis of T cell populations in small intestine lamina propria using flow cytometry.

Analysis of inflammatory markers, cytokines, and markers of epithelial integrity in the distal colon of mice after 4 weeks of supplementation indicate that the mice receiving the intervention oligosaccharides experience decreased gut inflammation, and thus, with potential application in gut health. Oligosaccharides having similar or substantially similar (as disclosed herein) structural and/or physical characteristics are expected to perform similarly.

### Mechanism 9—In Vitro Culture Model to Test the Effect of Oligosaccharides on 5-HT4 Receptor Activation

This mechanism pertains at least to gut health, gut-brain axis related diseases and metabolic and cardiovascular related diseases, conditions, disorders, and/or indications.

One such mechanism pertains to the production of enough of a given metabolite or group of metabolites to promoting motility and alleviating visceral pain. Specific microbial metabolites like tryptophan, among others, can promote motility and alleviate visceral pain, through interaction with 5-HT4 receptors in the gastrointestinal tract (Quigley 2011). Therefore, an increase in the relative abundance of microorganisms with the ability to produce molecules that act as agonists to these receptors is considered a therapeutic target for regulating gastrointestinal transit in patients having functional constipation, promoting gut health, gut-brain axis, and metabolic and cardiovascular related disease (Matsumoto et al. 2012, Bhattarai et al. 2018, Doggrell et al.2003).

To assess the ability of oligosaccharides to affect 5-HT4 receptors, we use Ussing chambers, with proximal colon tissues from WT mice (12 weeks of age) mounted in an Ussing chamber cassette (Physiologic Instrument). Changes in short circuit current are recorded before and after exposure to fecal supernatants (ΔIsc). Expression of 5-HT4 receptors is measured by Q-PCR.

Analysis of Q-PCR results shows that supernatants from fecal fermentations performed with the oligosaccharides CLX101, CLX 102, CLX 103, CLX105, CLX108, CLX109, CLX110, CLX111, CLX112, CLX113, CLX114, CLX115, CLX115AL, CLX107, CLX115a, CLX116, CLX117, CLX118, CLX119, CLX122, CLX122DS, CLX122DSF, CLX123, CLX124, CLX124, CLX126, CLX127, CLX128, CLX129, CLX130, XLX131, CLX132, and CLX133 increase expression of 5-HT4 receptor. Because of the impact of the activation of this receptor in the host, the oligosaccharides CLX101, CLX 102, CLX 103, CLX105, CLX108, CLX109, CLX110, CLX111, CLX112, CLX113, CLX114, CLX115, CLX115AL, CLX107, CLX115a, CLX116, CLX117, CLX118, CLX119, CLX122, CLX122DS, CLX122DSF, CLX123, CLX124, CLX124, CLX126, CLX127, CLX128, CLX129, CLX130, XLX131, CLX132, and CLX133 and those having similar or substantially similar (as disclosed herein) structural and/or physical characteristics could be used to modulate gut health, gut-brain axis related diseases and metabolic and cardiovascular diseases.

References for this mechanism are below, each of which is incorporated by reference in its entirety for all purposes:
Quigley. Microflora Modulation of Motility. J. Neurogastroenterol Motil (2011) 17:140
Matsumoto et al. Experimental colitis alters expression of 5-HT receptors and transient receptor potential vanilloid 1 leading to visceral hypersensitivity in mice. Lab Invest (2012) 92:769
Bhattarai et al. Gut microbiota produced tryptamine activates an epithelial G-Protein coupled receptor to increase colonic secretion. Cell host microbes (2018) 23:775
Doggrell et al. The role of 5-HT on the cardiovascular and renal systems and the clinical potential of 5-HT modulation. Expert Op Investig Drugs (2003) 12:805

### Mechanism 10—In Vivo Model to Test the Effect of Oligosaccharides on Total Gastrointestinal Transit Time

This mechanism pertains at least to gut-brain axis related diseases, conditions, disorders, and/or indications.

Red carmine dye assay is a common animal assay used to evaluate how the presence of specific metabolites in the gut can affect gut transit times and evaluate impact in gut-brain axis (Koester et al. 2021).

8-10 week old C57B1/6 mice are used to assess the effect of oligosaccharides on gastrointestinal transit time. Mice are divided in 30 groups (n=10) and fed with a standard diet during 21 days. During this period, all the groups of mice but the control group receive oligosaccharide in the drinking water (10% w/v). The intervention oligosaccharides are: CLX101, CLX 102, CLX 103, CLX105, CLX108, CLX109, CLX110, CLX111, CLX112, CLX113, CLX114, CLX115, CLX107, CLX115a, CLX116, CLX117, CLX118, CLX119, CLX122, CLX123, CLX124, CLX124, CLX126, CLX127, CLX128, CLX129, CLX130, CLX131, CLX132, CLX115AL, CLX122DS, CLX122DSF and CLX133. Mice are maintained on a strict 12 h light cycle (lights on between 6 am to 6 pm). Carmine red (Sigma-Aldrich) is prepared as a 6% (w/v) solution and gavaged at 8 am. Animals are not fasted beforehand. Feces are collected every 30 min up to 8 hours from time of gavage and evaluated to assay for the presence of the red carmine dye. The time from gavage to initial appearance of carmine in the feces is recorded as the total intestinal transit time for an animal.

Analysis of gastrointestinal transit time after 21 days of supplementation indicates that the mice receiving the intervention oligosaccharides CLX101, CLX 102, CLX 103, CLX105, CLX108, CLX109, CLX110, CLX111, CLX112, CLX113, CLX114, CLX115, CLX115AL, CLX107, CLX115a, CLX116, CLX117, CLX118, CLX119, CLX122, CLX122DS, CLX122DSF, CLX123, CLX124, CLX125, CLX126, CLX127, CLX128, CLX129, CLX130, XLX131, CLX132, and CLX133 experience shorter gastrointestinal transit time. Thus, these oligosaccharides and those having similar or substantially similar (as disclosed herein) structural and/or physical characteristics can be used as potential modulators of the gut-brain axis.

References for this mechanism are below, each of which is incorporated by reference in its entirety for all purposes:
Koester et al. Marker-based assay for studying gut transit in gnotobiotic and conventional mouse models. STAR Protocols (2021) 2.

### Mechanism 11—In Vitro Culture Model to Test the Effect of Oligosaccharides on FXR Activation.

This mechanism pertains at least to metabolic and cardiovascular related diseases, conditions, disorders, and/or indications.

One such mechanism pertains to the production of enough of a given metabolite or group of metabolites to reduce cholesterol levels. Specific microbial metabolites like ursodeoxycholic acid, 7-oxo-lithocholic acid and/or 7-oxo-deoxycholic, among others, can decrease the activation of FXR (Mi et al. 2003). Therefore, an increase in the relative abundance of microorganisms with the ability to increase molecules that act as agonists to these receptors is considered a therapeutic target for metabolic and cardiovascular disease, decreasing suppression of bile acid synthesis and increasing bile acid clearance, lowering circulating cholesterol (Jiang et al. 2015).

To assess the ability of oligosaccharides to affect FXR receptors, CHO cells are transfected with FXR responsive luciferase reporter (Indigo Biosciences), as described in Miyata et al. (2021). Cells are incubated for 22 h with supernatants from oligosaccharide-supplemented fecal fermentations. Supernatants were generated as described in Example 30 with oligosaccharides CLX101, CLX 102, CLX 103, CLX105, CLX108, CLX109, CLX110, CLX111, CLX112, CLX113, CLX114, CLX115, CLX107, CLX115a, CLX116, CLX117, CLX118, CLX119, CLX122, CLX123, CLX124, CLX125, CLX126, CLX127, CLX128, CLX129, CLX130, CLX131, CLX132, CLX115AL, CLX122DS, CLX122DSF and CLX133. Potency is assessed using a multi-mode microplate reader (Molecular Devices) for 24 h. Efficacies are reported relative to OCA, which was set as 100% FXR activation.

Analysis of FXR activity shows that supernatants from fecal fermentations performed with the oligosaccharides CLX101, CLX 102, CLX 103, CLX105, CLX108, CLX109, CLX110, CLX111, CLX112, CLX113, CLX114, CLX115, CLX115AL, CLX107, CLX115a, CLX116, CLX117, CLX118, CLX119, CLX122, CLX122DS, CLX122DSF, CLX123, CLX124, CLX125, CLX126, CLX127, CLX128, CLX129, CLX130, XLX131, CLX132, and CLX133 increase expression this receptor. Because of the impact of the activation of this receptor in the host, these oligosaccharides and those having similar or substantially similar (as disclosed herein) structural and/or physical characteristics could be used to modulate metabolic and cardiovascular disease

References for this mechanism are below, each of which is incorporated by reference in its entirety for all purposes:
Mi et al. Structural basis for bile acid binding and activation of the nuclear receptor FXR. Molecular Cell (2003) 11
Jiang et al. Intestine-selective farnesoid X receptor inhibition improves obesity-related metabolic dysfunction. Nature Communications (2015) 6
Miyata et al. Discovery, optimization and evaluation of non-bile acid FXR/TGR5 dual agonists. Sci Rep (2021) 11: 9196

### Mechanism 12—In Vivo Model to Test the Effect of Oligosaccharides in Alleviating Type 2 Diabetes Mellitus

This mechanism pertains at least to metabolic and cardiovascular related diseases, conditions, disorders, and/or indications.

Leptin-resistant db/db mice is a well-characterized murine model of T2DM as previously described (Cossio et al. 2017). db/db (4 weeks old) are used to evaluate the effect of oligosaccharides in spontaneous gut inflammation, and the impact in metabolic and cardiovascular diseases. Mice are divided in 30 groups (n=10) and fed with a standard diet during 6 weeks. During this period, all the groups of mice but the control group receive oligosaccharide in the drinking water (10% w/v). The intervention oligosaccharides are: CLX101, CLX 102, CLX 103, CLX105, CLX108, CLX109, CLX110, CLX111, CLX112, CLX113, CLX114, CLX115, CLX107, CLX115a, CLX116, CLX117, CLX118, CLX119, CLX122, CLX123, CLX124, CLX125, CLX126, CLX127, CLX128, CLX129, CLX130, CLX131, CLX132, CLX115AL, CLX122DS, CLX122DSF and CLX133. After 6 weeks of treatment, fresh stool samples are obtained. The animals are euthanized and blood samples are collected with EDTA-containing tubes and centrifuged to obtain plasma samples.

During the last week of the experiment, after fasting for 6 h, fasting blood glucose from one drop of tail blood is measured by using a standard glucometer. Body weight is also measured at the same time point. At the end of the experiment glycated hemoglobin (GHb) is measured using the Mouse Glycosylated Hemoglobin (GHb) ELISA kit. Biochemical indication of lipid metabolism, serum triglycerides and total cholesterol levels of each group is determined using AU4000 automatic biochemical analyzer. Plasma LPS levels in each group are measured using limulus amebocyte lysate kit (Xiamen Bioendo Technology). In addition, plasma IL-2, IL-4, IL-6, IL-17A, IL-10 and IFN-gamma in each group is measured by BD CBA Mouse Th1/Th2/Th17 cytokine kit (BD Bioscience).

Analysis of results after 6 weeks of supplementation indicate that the mice receiving the intervention oligosaccharides CLX101, CLX 102, CLX 103, CLX105, CLX108, CLX109, CLX110, CLX111, CLX112, CLX113, CLX114, CLX115, CLX115AL, CLX107, CLX115a, CLX116, CLX117, CLX118, CLX119, CLX122, CLX122DS, CLX122DSF, CLX123, CLX124, CLX125, CLX126, CLX127, CLX128, CLX129, CLX130, XLX131, CLX132, and CLX133 experience decrease in T2D markers. Thus, these oligosaccharides and those having similar or substantially similar (as disclosed herein) structural and/or physical characteristics could be used as potential modulation of metabolic and cardiovascular diseases.

References for this mechanism are below, each of which is incorporated by reference in its entirety for all purposes:
Cossio et al. Impact of prebiotics on metabolic and behavioral alterations in a mouse model of metabolic syndrome. Brain Behav Inmun (2017) 64:33

### Mechanism 13—In Vivo Model to Test the Effect of Oligosaccharides in Attenuation of Atherosclerosis

This mechanism pertains at least to metabolic and cardiovascular related diseases, conditions, disorders, and/or indications.

Atherosclerosis-prone apolipoprotein E-deficient (ApoE−/−) mice display poor lipoprotein clearance with subsequent accumulation of cholesterol ester-enriched particles in the blood, which promote the development of atherosclerotic plaques and cardiovascular diseases (Zhang et al. 2021).

ApoE−/− female mice (24 weeks old) are used to evaluate the effect of oligosaccharides in attenuation of vascular atherosclerosis. Mice are divided in 30 groups (n=10) and fed with a cholesterol-rich diet (1.25% cholesterol) diet during 16 weeks. After this period, mice are separated in groups, and all the groups of mice but the control group receive oligosaccharide in the drinking water (10% w/v). The intervention oligosaccharides are: CLX101, CLX 102, CLX 103, CLX105, CLX108, CLX109, CLX110, CLX111, CLX112, CLX113, CLX114, CLX115, CLX107, CLX115a, CLX116, CLX117, CLX118, CLX119, CLX122, CLX123, CLX124, CLX125, CLX126, CLX127, CLX128, CLX129, CLX130, CLX131, CLX132, CLX115AL, CLX122DS, CLX122DSF and CLX133. After 8 weeks of treatment, mice are fasted for 4 hours before blood and tissue collection. Plasma TMA, TMAO and creatinine levels are determined by mass spectrometry as previously described (Wang et al. 2011). For histological analysis, the left ventricle was perfused with 0.1M phosphate-buffered saline, followed by a 4% formaldehyde solution at a pressure of 100 mmHg. The aortic root and a portion of the ascendant aorta were embedded in OCT compound and cross-sectioned on a cryostat. For each animal, aorta cross-sections were mounted on gelatin-coated slides and stained with oil-Red-O (Sigma-Aldrich) to detect neutral lipids. Amount of TMA, TMAO in blood are lower in mice receiving the oligosaccharide treatment. Histological analysis of aortas shows a significant reduction in the lipid deposition area in mice receiving oligosaccharides.

Analysis of results after 16 weeks of supplementation indicate that the mice receiving the intervention oligosaccharides CLX101, CLX 102, CLX 103, CLX105, CLX108, CLX109, CLX110, CLX111, CLX112, CLX113, CLX114, CLX115, CLX115AL, CLX107, CLX115a, CLX116, CLX117, CLX118, CLX119, CLX122, CLX122DS, CLX122DSF, CLX123, CLX124, CLX125, CLX126, CLX127, CLX128, CLX129, CLX130, XLX131, CLX132, and CLX133 experience decrease of atherosclerosis and levels of TMA in blood. Thus, these oligosaccharides and those having similar or substantially similar (as disclosed herein) structural and/or physical characteristics could be used as potential modulation of metabolic and cardiovascular diseases.

References for this mechanism are below, each of which is incorporated by reference in its entirety for all purposes:
Zhang et al. Inhibition of microbiota-dependent TMAO production attenuates chronic kidney disease in mice. Scientific Reports (2021) 11:518
Wang et al. Gut flora metabolism of phosphatidylcholine promotes cardiovascular disease. Nature (2011) 472:57
   Mechanism 14: Oligosaccharide Structural Features that Facilitate Butyrate Production

Butyrate production by the microbiome is linked to several mechanisms that impact broad classes of diseases such as intestinal inflammatory diseases (ulcerative colitis, Chron's disease, Inflammatory bowel disorder), metabolic diseases (diabetes), and gut-brain axis disorders. Butyrate is created through fermentation pathways in the gut. Butyrate is known to be produced through several mechanisms including directly through butryl-kinase, but also through acetate, lactate, and succinate pathways. However, not all carbohydrate substrates are effectively fermented to butyrate by the gut microbiome. Carbohydrates may not be fermented into butyrate if a) there are structural restrictions that do not allow a carbohydrate to enter a pathway with butyrate as an end-product or if b) the carbohydrate modulates the microbiome away from butyrate producing microorganisms. Due to complications in predicting carbohydrate metabolism due to poorly annotated carbohydrate active enzymes and a lack of substrates to empirically measure metabolic changes, there has yet to be a method to predict butyrate production based upon carbohydrate structure. However, the oligosaccharide pools created herein offer substantially more structural heterogeneity than commercially available oligosaccharide materials and therefore can be used to model carbohydrate structure-metabolic function relationships. We assessed a structurally diverse set of oligosaccharide materials for their ability to be converted into butyrate by the microbiome and built a descriptive model of the carbohydrate structural requirements for butyrate fermentation.

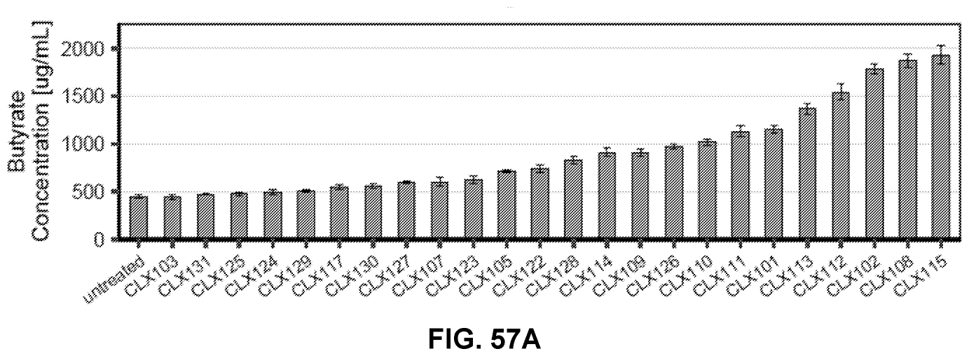

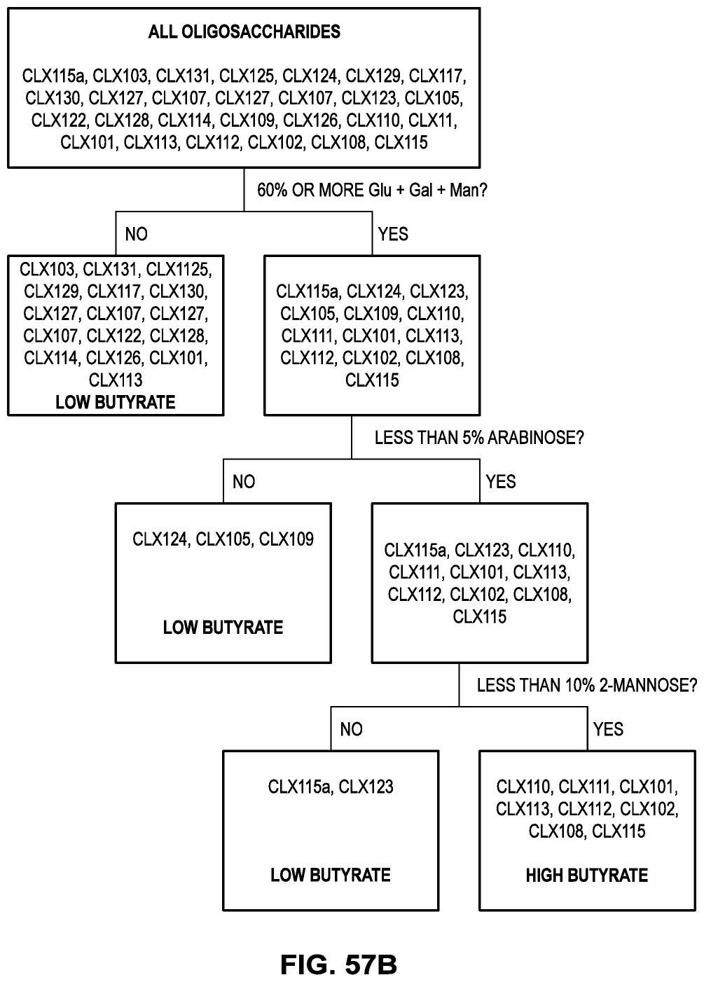

Oligosaccharide pools CLX115a, CLX103, CLX131, CLX125, CLX124, CLX129, CLX117, CLX130, CLX127, CLX107, CLX127, CLX107, CLX123, CLX105, CLX122, CLX128, CLX114, CLX109. CLX126, CLX110, CLX111, CLX101, CLX113, CLX112, CLX102, CLX108, CLX115 and a background carbohydrate control underwent fecal fermentations in triplicate as described in Example 13. Supernatant was sampled at 20 hours post-inoculation and analyzed in the manner described in Example 13. All samples except for CLX115a produced more butyrate than the background carbohydrate control. Butyrate concentrations ranged from 275.5 μg/ml to 1934.5 μg/ml with the average being 881.6 μg/ml. The butyrate concentrations of each CLX pool are shown in FIG. 57A.

To build the model the CLX pools were first categorically stratified into two groups based on whether they produced more or less than 1000 μg/ml butyrate. Next, a decision tree was designed that could accurately describe whether an oligosaccharide was a high or low butyrate producer. During the process, three features were found that generally distinguished between high and low butyrate producers (FIG. 57B). High butyrate producers had 1) at least 60 wt. % hexose subunits (e.g., a sum of glucose, galactose, and mannose subunits in an amount of at least 60 wt. %, based on total weight of saccharide subunits), 2) less than 5 wt. % arabinose subunits (e.g., a sum of arabinose subunits in an amount of less than 5 wt. %, based on total weight of saccharide subunits.), and 3) less than 10 wt. % 2-linked mannose subunits (e.g., less than 10 wt. % 2-linked mannose subunits, based on total weight of saccharide subunits.). The three features constitute a general framework to describe a genus of oligosaccharides that jointly share the ability to be converted into butyrate. The oligosaccharides that were not converted to butyrate were likely not able to be consumed by many of the butyrate producing bacteria in the gut or were more readily consumed by non-butyrate producing bacteria. High butyrate producing pools include CLX101, CLX102, CLX108, CLX110, CLX111, CLX112, CLX113, CLX 115.

Several other oligosaccharide pools produced moderate amounts of butyrate, which were assigned by a butyrate level of between 750 μg/ml and 1000 μg/ml. The moderate butyrate producing oligosaccharide pools include CLX109, CLX114, CLX122, CLX126, and CLX128. A unifying structural feature for these moderate butyrate producers include the presence of arabinose often accompanied by galactose.

Mechanism 15: Oligosaccharide Structural Features that Facilitate Propionate Production

Propionate production by the microbiome is linked to several mechanisms that impact broad classes of diseases such as intestinal inflammatory diseases (ulcerative colitis, Chron's disease, Inflammatory bowel disorder), metabolic diseases (diabetes), and gut-brain axis disorders. Propionate is created through fermentation pathways in the gut. Propionate is known to be produced through several mechanisms including lactate and succinate pathways, but also through amino acid fermentation pathways. However, not all carbohydrate substrates are effectively fermented to propionate by the gut microbiome. Carbohydrates may not be fermented into propionate if a) there are structural restrictions that do not allow a carbohydrate to enter a pathway with propionate as an end-product or if b) the carbohydrate modulates the microbiome away from propionate producing microorganisms. Due to complications in predicting carbohydrate metabolism due to poorly annotated carbohydrate active enzymes and a lack of substrates to empirically measure metabolic changes, there has yet to be a method to predict propionate production based upon carbohydrate structure. However, the oligosaccharide pools created herein offer substantially more structural heterogeneity than commercially available oligosaccharide materials and therefore can be used to model carbohydrate structure-metabolic function relationships. We assessed a structurally diverse set of oligosaccharide materials for their ability to be converted into Propionate by the microbiome and built a descriptive model of the carbohydrate structural requirements for propionate fermentation.

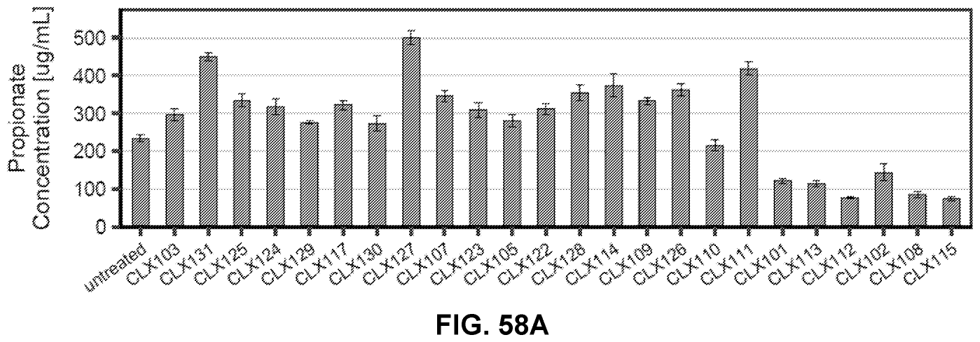

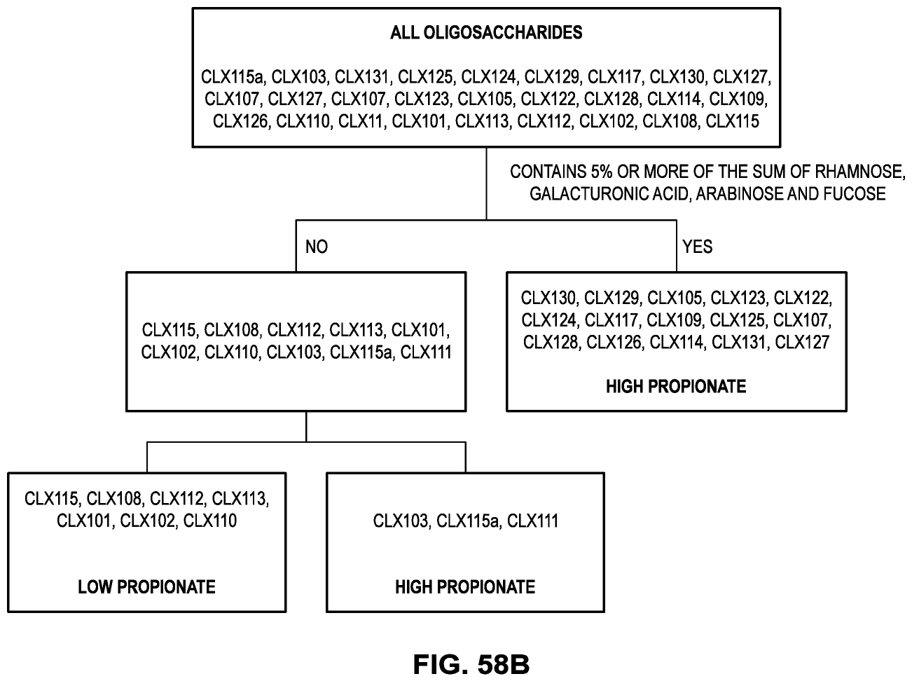

Oligosaccharide pools CLX115a, CLX103, CLX131, CLX125, CLX124, CLX129, CLX117, CLX130, CLX127, CLX107, CLX127, CLX107, CLX123, CLX105, CLX122, CLX128, CLX114, CLX109. CLX126, CLX110, CLX111, CLX101, CLX113, CLX112, CLX102, CLX108, CLX115 and a background carbohydrate control underwent fecal fermentations in triplicate as described in Example 13. Supernatant was sampled at 20 hours post-inoculation and analyzed in the manner described in Example 13. Several oligosaccharide pools, namely those highest in butyrate, produced less propionate than the untreated control. Propionate concentrations ranged from 75.5 μg/ml to 499.7 μg/ml with the average being 278.4 μg/ml. The Propionate concentrations of each CLX pool are shown in FIG. 58A.

To build the model we first categorically stratified the CLX pools into two groups based on whether they produced more or less than 275 μg/ml propionate. Next, a decision tree was designed that could accurately describe whether an oligosaccharide was a high or low propionate producer. During the process, a feature that distinguished between high and low Propionate producers was the presence of a sum of rhamnose, galacturonic acid, arabinose, fucose, and mannose subunits in an amount of at least 5 wt. %, based on total weight of saccharide subunits (FIG. 58B). However, pools that did not have this feature were less predictably assigned and would have to be determined on an empirical basis until better prediction methods can be developed. These features constitute a general framework to describe a genus of oligosaccharides that jointly share the ability to be converted into propionate. The oligosaccharides that were not converted to propionate were likely not able to be consumed by many of the propionate producing bacteria in the gut or were more readily consumed by non-propionate producing bacteria.

Mechanism 16: Oligosaccharide Structural Features that Facilitate Beta Hydroxybutyrate Production

Beta hydroxybutyrate production by the microbiome is linked to several mechanisms that impact broad classes of diseases such as intestinal inflammatory diseases (ulcerative colitis, Chron's disease, Inflammatory bowel disorder), metabolic diseases (diabetes), gut-brain axis disorders, epilepsy, and is the primary metabolite responsible for the benefits of the ketonic metabolic state. Beta hydroxybutyrate fermentation pathways in the gut are not well described. However, beta hydroxybutyrate may be produced through acetic acid intermediates. However, only few carbohydrate substrates are effectively fermented to beta hydroxybutyrate by the gut microbiome. Carbohydrates may not be fermented into beta hydroxybutyrate if a) there are structural restrictions that do not allow a carbohydrate to enter a pathway with beta hydroxybutyrate as an end-product or if b) the carbohydrate modulates the microbiome away from beta hydroxybutyrate producing microorganisms. Due to complications in predicting carbohydrate metabolism due to poorly annotated carbohydrate active enzymes and a lack of substrates to empirically measure metabolic changes, there has yet to be a method to predict beta hydroxybutyrate production based upon carbohydrate structure. However, the oligosaccharide pools created herein offer substantially more structural heterogeneity than commercially available oligosaccharide materials and therefore can be used to model carbohydrate structure-metabolic function relationships. We assessed a structurally diverse set of oligosaccharide materials for their ability to be converted into beta hydroxybutyrate by the microbiome and built a descriptive model of the carbohydrate structural requirements for beta hydroxybutyrate fermentation.

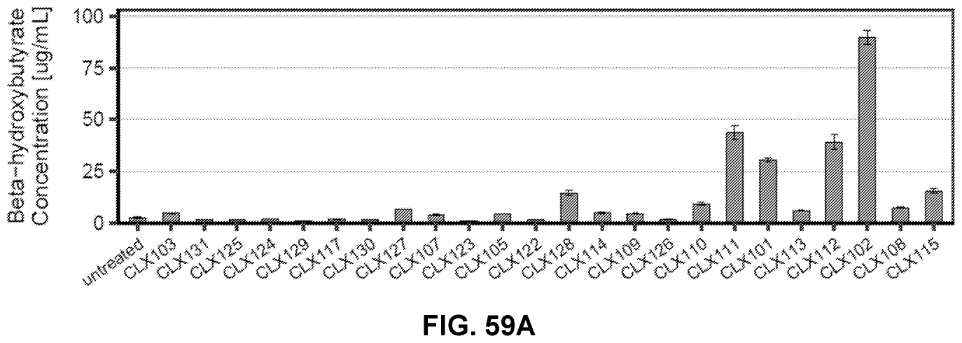

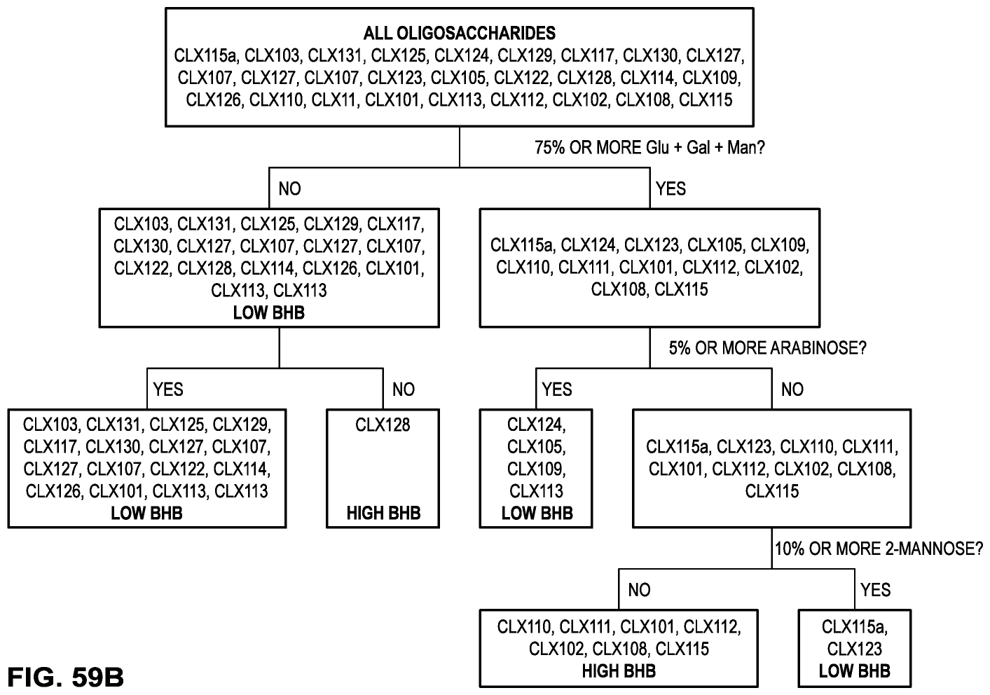

Oligosaccharide pools CLX115a, CLX103, CLX131, CLX125, CLX124, CLX129, CLX117, CLX130, CLX127, CLX107, CLX127, CLX107, CLX123, CLX105, CLX122, CLX128, CLX114, CLX109. CLX126, CLX110, CLX111, CLX101, CLX113, CLX112, CLX102, CLX108, CLX115 and a background carbohydrate control underwent fecal fermentations in triplicate as described in Example 13. Supernatant was sampled at 20 hours post-inoculation and analyzed in the manner described in Example 13. Many oligosaccharides pools, produced measurable levels of beta hydroxybutyrate, however, only few produced substantially high amounts. Beta hydroxybutyrate concentrations reached as high as 89.7 μg/ml with the average being 11.5 μg/ml. The beta hydroxybutyrate concentrations of each CLX pool are shown in FIG. 59A.

To build the model first the CLX pools were categorically stratified into two groups based on whether they produced more or less than 7 μg/ml propionate. Next, a decision tree was designed that could accurately describe whether an oligosaccharide was a high or low beta hydroxybutyrate producer. During the process, three features were found that distinguished between high and low butyrate producers (FIG. 59B). High beta hydroxybutyrate producers had 1) at least 75 wt. % hexose subunits (e.g., a sum of glucose, galactose, mannose subunits in an amount of at least 75 wt. %, based on total weight of saccharide subunits), 2) less than 5 wt. % arabinose subunits (e.g., a sum of arabinose subunits in an amount of less than 5 wt. %, based on total weight of saccharide subunits), and 3) less than 10 wt. % 2-linked mannose subunits (e.g., less than 10 wt. % 2-linked mannose subunits, based on total weight of saccharide subunits). However, one pool that this workflow would have incorrectly predicted as a low beta hydroxybutyrate producer was pool CLX128. The production of beta hydroxybutyrate by CLX128 demonstrates that while the model only identified correct assignments, it may miss false-negatives, which demonstrates it to be a conservative model. Therefore, negative predictions could produce beta hydroxybutyrate but would need to be evaluated on an empirical basis until better prediction methods are be developed. The three features constitute a general framework to describe a genus of oligosaccharides that jointly share the ability to be converted into beta hydroxybutyrate. The oligosaccharides that were not converted to beta hydroxybutyrate were likely not able to be consumed by many of the beta hydroxybutyrate producing bacteria in the gut or were more readily consumed by non-beta hydroxybutyrate producing bacteria. High beta hydroxybutyrate producing pools include CLX110, CLX111, CLX101 CLX112, CLX102, CLX108, CLX115, CLX128.

Mechanism 17: Oligosaccharides that Facilitate the Production of Bioactive Indole Derivatives

Indole derivate production by the microbiome is linked to several mechanisms that impact broad classes of diseases such as intestinal inflammatory diseases (ulcerative colitis, Chron's disease, Inflammatory bowel disorder), metabolic diseases (diabetes), and gut-brain axis disorders. Indole derivatives are derived through tryptophan metabolic pathways in the gut. However, not all bacteria in the gut can convert tryptophan into indole and its derivatives (indole-3-lactate, indole-3-acrylate, indole-3-acetate, indole-3-acetalaldehyde, indole-3-pyruvate, indole-3-propionate, and indole-3-carboxaldehyde). Tryptophan may not be fermented into indole derivatives if the carbohydrate modulates the microbiome away from indole derivate producing microorganisms such as Bifidobacterium. Due to complications in predicting carbohydrate modulation of indole derivative converting bacteria, which is due to poorly annotated carbohydrate active enzymes and a lack of substrates to empirically measure metabolic changes, there has yet to be a method to predict indole derivate production based upon carbohydrate structure. However, the oligosaccharide pools presented herein, or structurally similar oligosaccharide pools, have not previously been tested for their indole derivative production capacity and therefore give a glimpse into the structural characteristics of carbohydrates that may modulate indole derivative converting bacteria and thus indole derivative levels in the gut. We assessed a structurally diverse set of oligosaccharide materials for their ability to be converted into indole derivatives by the microbiome and describe the carbohydrate structural features that can promote indole derivative production.

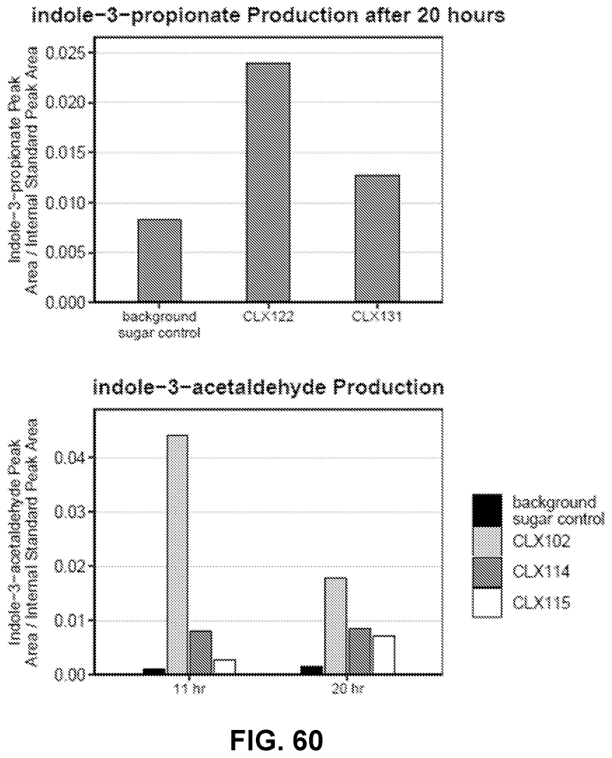

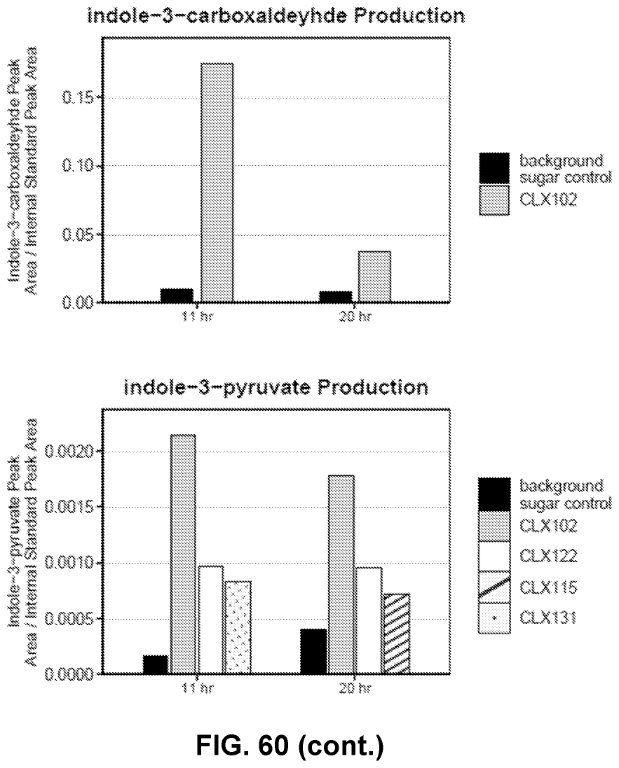

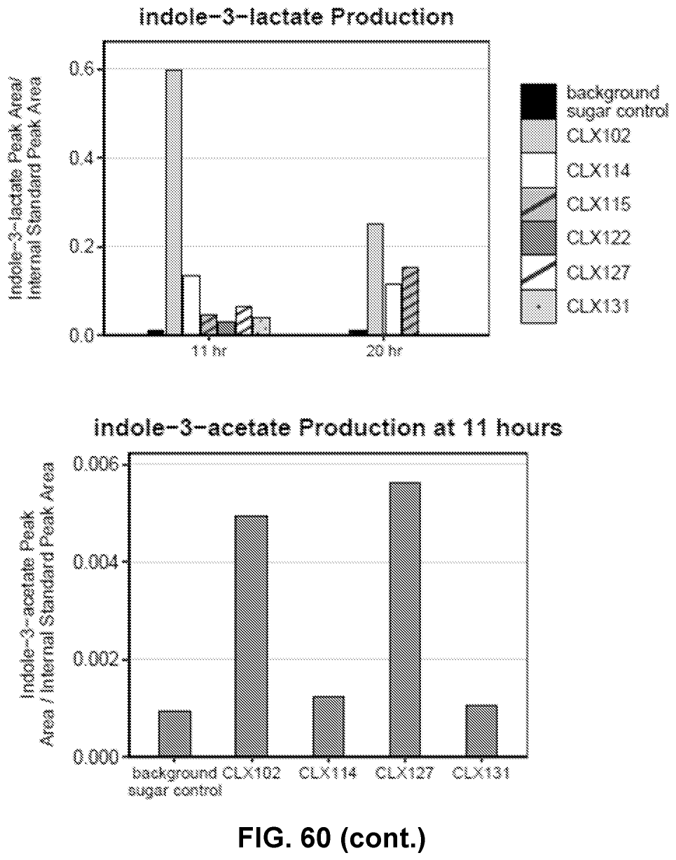

Oligosaccharide pools CLX102, CLX114, CLX115, CLX122, CLX131, CLX127 and a background carbohydrate control underwent fecal fermentations in triplicate as described in Example 13. Supernatant was sampled at 11 and 20 hours post-inoculation and analyzed in the manner described in Example 13. All of the selected oligosaccharides pools produced measurable levels of indole derivatives but of varying types and at varying time points. For example, CLX102 produced the most indole-3-carboxaldeyhde at both 11 and 21 hours. CLX131 and CLX122 produced more indole-3-propionate than the background sugar control at 20 hours. CLX102, CLX131, and CLX122 produced more indole-3-pyruvate than the background sugar control at 11 hours. CLX102, CLX115, and CLX122 produced more indole-3-pyruvate than the background sugar control at 20 hours. CLX102, CLX115, and CLX114 produced more indole-3-acetylalehyde than the background sugar control at 11 and 20 hours. CLX102, CLX127, CLX131, and CLX114 produced more indole-3-acetate than the background sugar control at 11 hours. All CLX tested produced more indole-3-lactate than the background sugar control at 11 hours. CLX102, CLX115, CLX114 produced more indole-3-lactate than the background sugar control at 20 hours. All data is shown in FIG. 60. The oligosaccharides that were not converted to indole derivates were likely not able to be consumed by many of the indole derivates producing bacteria in the gut or were more readily consumed by non-indole derivates producing bacteria.

Mechanism 18: Oligosaccharides that Facilitates Metabolic Conversion of Bile Acids and/or Bile Salts

Bile acid and/or bile salt metabolism by the microbiome is linked to several mechanisms that impact broad classes of diseases such as intestinal inflammatory diseases (ulcerative colitis, Chron's disease, Inflammatory bowel disorder), metabolic diseases (diabetes), and gut-brain axis disorders. Bile acid metabolism involves the conversion of conjugated primary bile acids (taurocholic acid, glycocholic acid, taurochenodeoxycholic acid and glycochenodeoxycholic acid) to their unconjugated forms (cholic acid, chenodeoxycholic acid) into secondary bile acids (lithocholic acid, chenodeoxycholic acid) and ultimately onto a variety of further oxidated products (ursodeoxycholic acid, 7-oxo-lithocholic acid, 7-oxo-deoxycholatic acid, and others). The bacteria in the gut are potent converters of bile acids and are partially responsible for bile-acid reuptake. However, not all bacteria in the gut metabolize bile acids. Bile acid metabolism may not occur if the carbohydrate modulates the microbiome away from bile acid metabolizing microorganisms such as Clostridium clusters XIVa and cluster XI members and certain Eubacterium. Due to complications in predicting carbohydrate modulation of bile acid metabolizing bacteria, which is due to poorly annotated carbohydrate active enzymes and a lack of substrates to empirically measure metabolic changes, there has yet to be a method to predict bile acid metabolism based upon carbohydrate structure. However, the oligosaccharide pools presented herein, or structurally similar oligosaccharide pools, have not previously been tested for their bile acid metabolizing capacity and therefore give a glimpse into the structural characteristics of carbohydrates that may modulate bile acid metabolizing bacteria and thus bile acid metabolizing levels in the gut. We assessed a structurally diverse set of oligosaccharide materials for their ability to modulate bile acid metabolisms by the microbiome and describe the carbohydrate structural features that can promote bile acid metabolism.

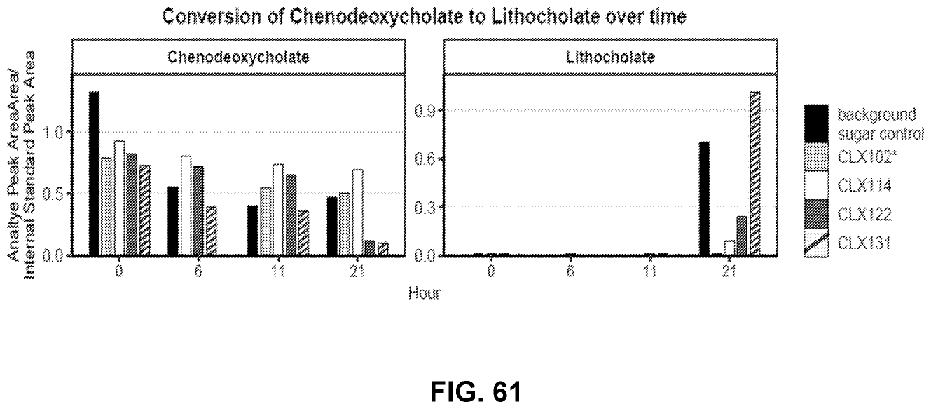

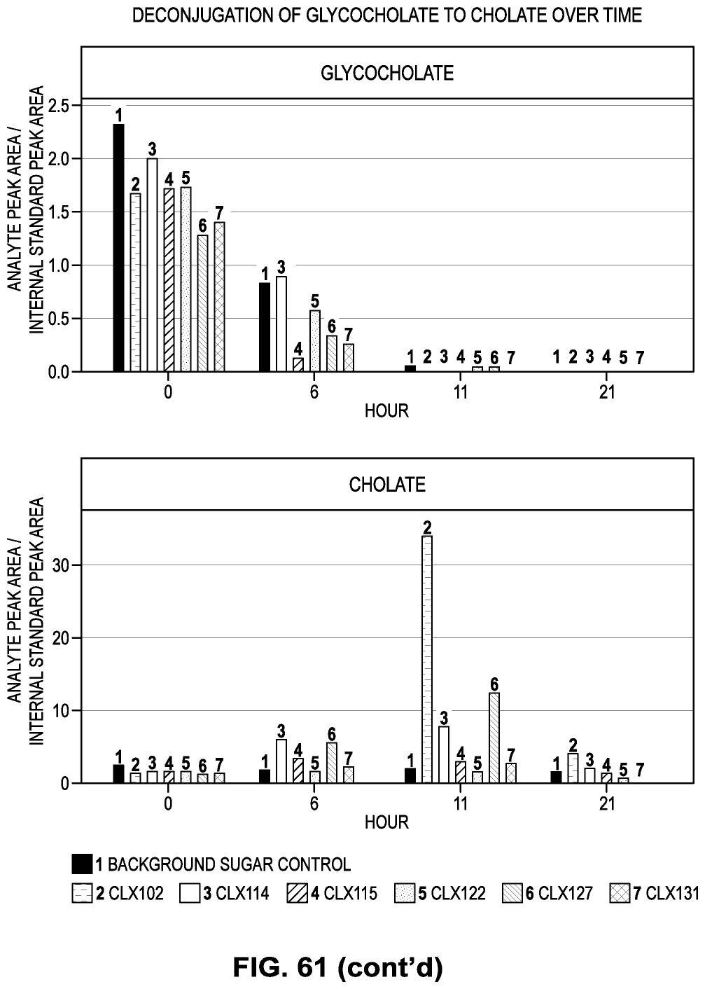

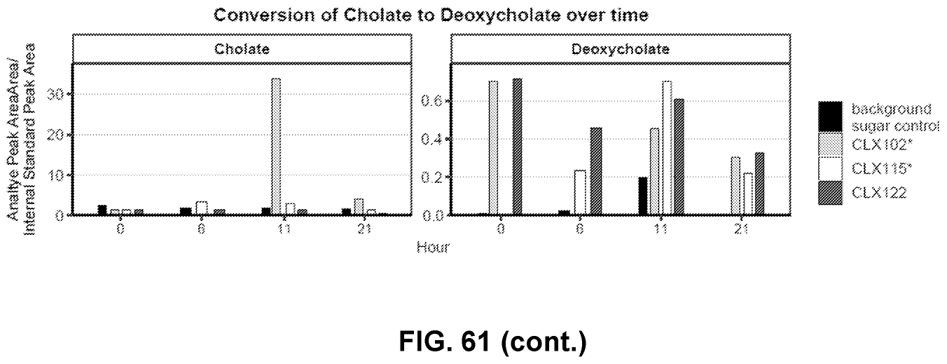

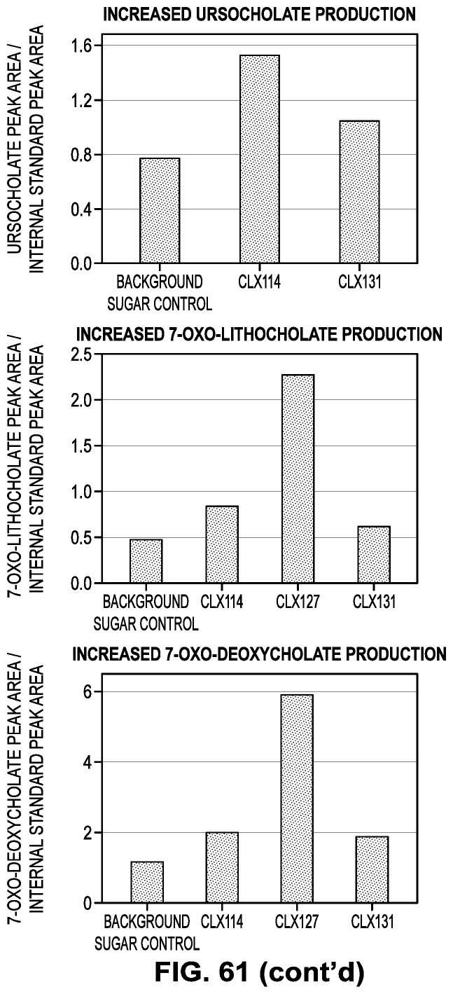

Oligosaccharide pools CLX102, CLX114, CLX115, CLX122, CLX131, CLX127 and a background carbohydrate control underwent fecal fermentations in triplicate as described in Example 13. Supernatant was sampled at 0, 6, 11, and 20 hours post-inoculation and analyzed in the manner described in Example 13. All of the selected oligosaccharides pools induced some degree of bile acid metabolisms but of varying types and at varying timepoints. For example, CLX102, CLX131, CLX122, and CLX114 all converted chenodeoxycholate to lithocholate across the fermentation period. CLX127, CLX102, CLX122 CLX115, CLX131, and CLX114 all deconjugated glycocholate to cholate faster than the untreated control. CLX102, CLX115, CLX122 all converted cholate to deoxycholate faster than the untreated control. CLX114 and CLX131 produced more ursocholate than the untreated control. CLX127, CLX114, and CLX131 produced more 7-oxo-lithocholate than the untreated control. CLX127, CLX114, CLX122, CLX131 produced more 7-oxo-deoxycholate than the untreated control. All data is shown in FIG. 61.

Various aspects are contemplated herein, several of which are set forth in the paragraphs below. It is explicitly contemplated that any aspect or portion thereof can be combined to form an aspect. In addition, it is explicitly contemplated that any aspect (e.g., Aspect A13) that references an aspect (e.g., Aspect A1) for which there are sub-aspects having the same top level number (e.g., Aspect A1a, A1b, A1c, and so forth) necessarily includes reference to those sub-aspects A1a, A1b, A1c, and so forth. In other words, if Aspect A13 refers to Aspect A1, and there are Aspects A1a and A1b present, then Aspect A13 refers to Aspects A1a or A1b. Furthermore, although the aspects below are subdivided into aspects A, B, C, D, and so forth, it is explicitly contemplated that aspects in each of subdivisions A, B, C, D, etc. can be combined in any manner. Moreover, the term “any preceding aspect” means any aspect that appears prior to the aspect that contains such phrase (in other words, the sentence “Aspect B13: The method of any one of aspeccts B1-B12, or any preceding aspect, . . . ” means that any aspect prior to aspect B13 is referenced, including aspects B1-B12 and all of the “A” aspects). For example, it is contemplated that, optionally, any method or composition of any the below aspects may be useful with or combined with any other aspect provided below. Further, for example, it is contemplated that any embodiment described elsewhere herein, including above this paragraph, may optionally be combined with any of the below listed aspects. Within the aspects described herein, including those aspects set forth below, any “comprising” term (and grammatical variations thereof) can be replaced by “collectively comprising” (and grammatical variations thereof). In this regard, any structural feature of one or more oligosaccharides, or an oligosaccharide composition, can be described in terms of “comprising” or “collectively comprising” (and grammatical variations thereof). In some instances in the aspects below, or elsewhere herein, two open ended ranges are disclosed to be combinable into a range. For example, “at least X” is disclosed to be combinable with “less than Y” to form a range, in which X and Y are numeric values. For the purposes of forming ranges herein, it is explicitly contemplated that “at least X” combined with “less than Y” forms a range of X-Y inclusive of value X and value Y, even through “less than Y” in isolation does not include Y.

Aspect A1: A method for modulating microbiota to produce at least one short chain fatty acid and/or to increase an abundance of the microbiota, the method comprising:
contacting the microbiota with a formulation comprising an oligosaccharide composition;
wherein the method modulates the microbiota to produce the at least one short chain fatty acid and/or increase the abundance of the microbiota; and
wherein the oligosaccharide composition comprises:
   (1) at least one first feature comprising:
      a sum of glucose, galactose, and mannose subunits in an amount of at least 40 wt. % (e.g., at least any of the following: 45, 50, 55, 60, 65, 70, 75, 80, 85, 90, 95, or 99 wt. %; optionally 100 wt. %; optionally less than any of the following: 100, 99, 95, 90, 85, 80, 75, 70, 65, 60, 55, 50, or 45 wt. %; optionally wherein any of such values can be combined in any manner to form a range, such as 60-99 wt. %), based on total weight of saccharide subunits; or
   (2) at least one second feature comprising:
      a sum of rhamnose, galacturonic acid, arabinose, fucose, and mannose subunits in an amount of at least 3 wt. % (e.g., at least any of the following: 4, 5, 7, 10, 12, 15, 20, 25, or 30 wt. %; optionally less than any of the following: 35, 30, 25, 20, 15, 12, 10, 7, 5, or 4 wt. %; optionally wherein any of such values can be combined in any manner to form a range, such as 5-10 wt. %), based on total weight of saccharide subunits;
      a sum of xylose subunits in an amount of at least 33 wt. % (e.g., at least any of the following: 35, 40, 45, 50, 55, 60, 65, 70, 75, 80, 85, or 90 wt. %; optionally less than any of the following: 95, 90, 85, 80, 75, 70, 65, 60, 55, 50, 45, 40, or 35 wt. %; optionally wherein any of such values can be combined in any manner to form a range, such as 33-55 wt. %), based on total weight of saccharide subunits;
      a sum of mannose subunits in an amount of at least 50 wt. % (e.g., at least any of the following: 50, 55, 60, 65, 70, 75, 80, 85, 90, 95, or 99 wt. %; optionally 100 wt. %; optionally less than any of the following: 100, 99, 95, 90, 85, 80, 75, 70, 65, 60, or 55 wt. %; optionally wherein any of such values can be combined in any manner to form a range, such as 55-90 wt. %), based on total weight of saccharide subunits; and a sum of galactose subunits in an amount of less than 20 wt. % (e.g., less than any of the following: 15, 10, 5 wt. %; optionally at least any of the following: 1, 5, or 10 wt. %; optionally wherein any of such values can be combined in any manner to form a range, such as 1-15 wt. %), based on total weight of saccharide subunits;
      a weight ratio of mannose subunits to glucose subunits between 2:1 and 4:1 (e.g., 2:1 to 2.5:1, 2:1 to 3:1, 2:1 to 3.5:1, 2.5:1 to 3:1, 2.5:1 to 3.5:1, 2.5:1 to 4:1, 3:1 to 3.5:1, 3:1 to 4:1, or 3.5:1 to 4:1); and wherein at least 70 wt. % (e.g., at least any of the following: 75, 80, 85, 90, 95, 97, or 99 wt. %; optionally essentially all; optionally 100 wt. %; optionally less than any of the following: 100, 99, 97, 95, 90, 85, 80, or 75 wt. %; optionally where any such values can be combined in any manner to form a range, such as 85-99 wt. % or 90 wt. % to essentially all) of the non-terminal mannose subunits have at least one 2-linkage, and wherein at least 70 wt. % (e.g., at least any of the following: 75, 80, 85, 90, 95, 97, or 99 wt. %; optionally essentially all; optionally 100 wt. %; optionally less than any of the following: 100, 99, 97, 95, 90, 85, 80, or 75 wt. %; optionally where any such values can be combined in any manner to form a range, such as 90-99 wt. % or 95 wt. % to essentially all) of the non-terminal glucose subunits have at least one 3-linkage, at least one 4-linkage, and/or at least one 6-linkage; or
      any combination thereof; or
   (3) at least one third feature comprising:
      a sum of glucose, galactose, mannose subunits in an amount of at least 50 wt. % (e.g., at least any of the following: 50, 55, 60, 65, 70, 75, 80, 85, 90, 95, or 99 wt. %; optionally 100 wt. %; optionally less than any of the following: 100, 99, 95, 90, 85, 80, 75, 70, 65, 60, or 55 wt. %; optionally wherein any of such values can be combined in any manner to form a range, such as 75-99 wt. %), based on total weight of saccharide subunits;
      a sum of glucose subunits in an amount of at least 20 wt. % (e.g., at least any of the following: 22, 24, 26, 28, 30, 32, 35, 37, 40, 42, 45, or 47 wt. %; optionally less than any of the following: 50, 47, 45, 42, 40, 37, 35, 32, 30, 28, 26, or 24 wt. %; optionally wherein any of such values can be combined in any manner to form a range, such as 20-40 wt. %), based on total weight of saccharide subunits; and a sum of xylose and arabinose subunits in an amount of at least 33 wt. % (e.g., at least any of the following: 35, 40, 45, 50, 55, 60, 65, 70, 75, 80, 85, or 90 wt. %; optionally less than any of the following: 95, 90, 85, 80, 75, 70, 65, 60, 55, 50, 45, 40, or 35 wt. %; optionally wherein any of such values can be combined in any manner to form a range, such as 33-55 wt. %), based on total weight of saccharide subunits; or
      any combination thereof.

Aspect A2: The method of aspect A1, or any preceding aspect, wherein the oligosaccharide composition comprises at least 50 wt. % (e.g., at least any of the following: 50, 55, 60, 65, 70, 75, 80, 85, 90, 95, or 99 wt. %; optionally 100 wt. %; optionally less than any of the following: 100, 99, 95, 90, 85, 80, 75, 70, 65, 60, or 55 wt. %; optionally wherein any of such values can be combined in any manner to form a range, such as 70-99 wt. %) oligosaccharides on a dry basis having a degree of polymerization of between 3 and 50 (e.g., 3-5, 3-8, 3-10, 3-12, 3-15, 3-18, 3-20, 3-25, 3-30, 3-35, 3-40, 3-45, 5-8, 5-10, 5-12, 5-15, 5-18, 5-20, 5-25, 5-30, 5-35, 5-40, 5-45, 5-50, 10-15, 10-20, 10-25, 10-30, 10-35, 10-40, 10-45, 10-50, 15-20, 15-25, 15-30, 15-35, 15-40, 15-45, 15-50, 20-25, 20-30, 20-35, 20-40, 20-45, 20-50, 25-30, 25-35, 25-40, 25-45, 25-50, 30-35, 30-40, 30-45, 30-50, 35-40, 35-45, 35-50, 40-45, 40-50, or 45-50) monosaccharide subunits.

Aspect A3: The method of aspect A1 or A2, or any preceding aspect, wherein the oligosaccharide composition comprises a dynamic viscosity at 25° C. of between 0.8 and 2.5 (e.g., 0.8-1, 0.8-1.2, 0.8-1.5, 0.8-1.7, 0.8-2, 0.8-2.2, 1-1.2, 1-1.5, 1-1.7, 1-2, 1-2.2, 1-2.5, 1.2-1.5, 1.2-1.7, 1.2-2, 1.2-2.2, 1.2-2.5, 1.5-1.7, 1.5-2, 1.5-2.2, 1.5-2.5, 1.7-2, 1.7-2.2, 1.7-2.5, 2-2.2, 2-2.5, or 2.2-2.5) mPa*s at a concentration of 100 mg of the oligosaccharide composition on a dry basis in 1 mL (i.e., per mL) of water.

Aspect A4: The method of any one of aspects A1-A3, or any preceding aspect, wherein the oligosaccharide composition comprises less than 5 wt. % (e.g., less than any of the following: 4, 3, 2, 1, or 0.5 wt. %; optionally at least any of the following: 0.1, 0.5, 1, 2, 3, or 4 wt. %; optionally wherein any of such values can be combined in any manner to form a range, such as 0.1-3 wt. %) monosaccharides.

Aspect A5: The method of any one of aspects A1-A4, or any preceding aspect, wherein the oligosaccharide composition comprises the at least one first feature.

Aspect A6: The method of aspect A5, or any preceding aspect, wherein the oligosaccharide composition comprises:
a sum of arabinose subunits in an amount of less than 8 wt. % (e.g., less than any of the following: 8, 7, 6, 5, 4, 3, 2, 1, or 0.5 wt. %; optionally at least any of the following: 0.1, 0.5, 1, 2, 3, 4, 5, 6, or 7 wt. %; optionally wherein any of such values can be combined in any manner to form a range, such as 3-5 wt. %), based on total weight of saccharide subunits.

Aspect A7: The method of aspect A5 or A6, or any preceding aspect, wherein the oligosaccharide composition comprises:
less than 15 wt. % (e.g., less than any of the following: 15, 12, 10, 7, 5, or 3 wt. %; optionally at least any of the following: 1, 3, 5, 7, 10, or 12 wt. %; optionally wherein any of such values can be combined in any manner to form a range, such as 7-10 wt. %) 2-linked mannose subunits, based on total weight of saccharide subunits.

Aspect A8a: The method of aspect A7, or any preceding aspect, wherein the 2-linked mannose subunits are β1,2 linked.

Aspect A8b: The method of any one of aspects A5-A8a, or any preceding aspect, wherein the oligosaccharide composition comprises non-terminal glucose subunits, wherein (1) 20 wt. % to 100 wt. % (e.g., 20-30, 20-40, 20-50, 20-60, 20-70, 20-80, 20-90, 20-95, 20-99, 30-40, 30-50, 30-60, 30-70, 30-80, 30-90, 30-95, 30-99, 30-100, 40-50, 40-60, 40-70, 40-80, 40-90, 40-95, 40-99, 40-100, 50-60, 50-70, 50-80, 50-90, 50-95, 50-99, 50-100, 60-70, 60-80, 60-90, 60-95, 60-99, 60-100, 70-80, 70-90, 70-95, 70-99, 70-100, 80-90, 80-95, 80-99, 80-100, 90-95, 90-99, 90-100, 95-99, 95-100, or 99-100 wt. %) of the non-terminal glucose subunits have at least one 3-linkage, based on total weight of non-terminal saccharide subunits; and/or (2) 33 wt. % to 80 wt. % (e.g., 33-50, 33-60, 33-70, 40-50, 40-60, 40-70, 40-80, 50-60, 50-70, 50-80, 60-70, 60-80, or 70-80 wt. %) of the non-terminal glucose subunits have at least one 4-linkage, based on total weight of non-terminal saccharide subunits.

Aspect A8c: The method of any one of aspects A5-A8b, or any preceding aspect, wherein the oligosaccharide composition comprises non-terminal glucose subunits, wherein (1) 20 wt. % to 100 wt. % (e.g., 20-30, 20-40, 20-50, 20-60, 20-70, 20-80, 20-90, 20-95, 20-99, 30-40, 30-50, 30-60, 30-70, 30-80, 30-90, 30-95, 30-99, 30-100, 40-50, 40-60, 40-70, 40-80, 40-90, 40-95, 40-99, 40-100, 50-60, 50-70, 50-80, 50-90, 50-95, 50-99, 50-100, 60-70, 60-80, 60-90, 60-95, 60-99, 60-100, 70-80, 70-90, 70-95, 70-99, 70-100, 80-90, 80-95, 80-99, 80-100, 90-95, 90-99, 90-100, 95-99, 95-100, or 99-100 wt. %) of the non-terminal glucose subunits are β1-3 linked, based on total weight of non-terminal saccharide subunits; and/or (2) 33 wt. % to 80 wt. % (e.g., 33-50, 33-60, 33-70, 40-50, 40-60, 40-70, 40-80, 50-60, 50-70, 50-80, 60-70, 60-80, or 70-80 wt. %) of the non-terminal glucose subunits are β1-4 linked, based on total weight of non-terminal saccharide subunits.

Aspect A8d: The method of any one of aspects A5-A8c, or any preceding aspect, wherein the oligosaccharide composition comprises:
non-terminal glucose subunits, wherein (1) 20 wt. % to 100 wt. % (e.g., 20-30, 20-40, 20-50, 20-60, 20-70, 20-80, 20-90, 20-95, 20-99, 30-40, 30-50, 30-60, 30-70, 30-80, 30-90, 30-95, 30-99, 30-100, 40-50, 40-60, 40-70, 40-80, 40-90, 40-95, 40-99, 40-100, 50-60, 50-70, 50-80, 50-90, 50-95, 50-99, 50-100, 60-70, 60-80, 60-90, 60-95, 60-99, 60-100, 70-80, 70-90, 70-95, 70-99, 70-100, 80-90, 80-95, 80-99, 80-100, 90-95, 90-99, 90-100, 95-99, 95-100, or 99-100 wt. %) of the non-terminal glucose subunits have at least one 3-linkage, based on total weight of non-terminal saccharide subunits; and/or (2) 33 wt. % to 80 wt. % (e.g., 33-50, 33-60, 33-70, 40-50, 40-60, 40-70, 40-80, 50-60, 50-70, 50-80, 60-70, 60-80, or 70-80 wt. %) of the non-terminal glucose subunits have at least one 4-linkage, based on total weight of non-terminal saccharide subunits; and
a weight ratio of glucose subunits having at least one 4-linkage to glucose subunits having at least one 3-linkage is between 2:1 to 4:1 (e.g., 2:1 to 2.5:1, 2:1 to 3:1, 2:1 to 3.5:1, 2.5:1 to 3:1, 2.5:1 to 3.5:1, 2.5:1 to 4:1, 3:1 to 3.5:1, 3:1 to 4:1, or 3.5:1 to 4:1).

Aspect A8e: The method of any one of aspects A5-A8d, or any preceding aspect, wherein the oligosaccharide composition comprises a weight ratio of glucose subunits having at least one 4-linkage to glucose subunits having at least one 3-linkage of between 2:1 to 4:1 (e.g., 2:1 to 2.5:1, 2:1 to 3:1, 2:1 to 3.5:1, 2.5:1 to 3:1, 2.5:1 to 3.5:1, 2.5:1 to 4:1, 3:1 to 3.5:1, 3:1 to 4:1, or 3.5:1 to 4:1).

Aspect A8f: The method of any one of aspects A5-A8e, or any preceding aspect, wherein the oligosaccharide composition comprises a weight ratio of glucose subunits having β1-4 linkages to glucose subunits having P1-3 linkages of between 2:1 to 4:1 (e.g., 2:1 to 2.5:1, 2:1 to 3:1, 2:1 to 3.5:1, 2.5:1 to 3:1, 2.5:1 to 3.5:1, 2.5:1 to 4:1, 3:1 to 3.5:1, 3:1 to 4:1, or 3.5:1 to 4:1).

Aspect A8g: The method of any one of aspects A5-A8f, or any preceding aspect, wherein the oligosaccharide composition comprises non-terminal glucose subunits, wherein 30 wt. % to 45 wt. % (e.g., 30-32, 30-35, 30-37, 30-40, 30-42, 32-35, 32-37, 32-40, 32-42, 32-45, 35-37, 35-40, 35-42, 35-45, 37-40, 37-42, 37-45, 40-42, 40-45, or 42-45 wt. %) of the non-terminal glucose subunits are β1-3 linked, based on total weight of non-terminal saccharide subunits.

Aspect A8h: The method of any one of aspects A5-A8g, or any preceding aspect, wherein the oligosaccharide composition comprises at least one of (e.g., at least two of, at least three of, at least four of, at least five of, at least six of, at least seven of, or at least eight of): (a) at least two (e.g., at least 3, 4, 5, 6, 7, 8, or 9; optionally less than 10, 9, 8, 7, 6, 5, 4, or 3; optionally wherein any of such values can be combined in any manner to form a range, such as 2-9) different isomers of 3Hex, (b) at least three (e.g., at least 3, 4, 5, 6, 7, 8, or 9; optionally less than 10, 9, 8, 7, 6, 5, or 4; optionally wherein any of such values can be combined in any manner to form a range, such as 3-7) different isomers of 3Hex, (c) at least four (e.g., at least 4, 5, 6, 7, 8, 9, 10, 11, 12, 13, 14, 15; optionally less than 16, 15, 14, 13, 12, 11, 10, 9, 8, 7, 6, or 5; optionally wherein any of such values can be combined in any manner to form a range, such as 8-16) different isomers of 4Hex, (d) at least five (e.g., at least 5, 6, 7, 8, 9, 10, 11, 12, 13, 14, 15; optionally less than 16, 15, 14, 13, 12, 11, 10, 9, 8, 7, or 6; optionally wherein any of such values can be combined in any manner to form a range, such as 5-12) different isomers of 5Hex, (e) at least three (e.g., at least 3, 4, 5, 6, 7, 8, or 9; optionally less than 10, 9, 8, 7, 6, 5, or 4; optionally wherein any of such values can be combined in any manner to form a range, such as 3-7) different isomers of 6Hex, or (f) any combination thereof.

Aspect A8i: The method of any one of aspects A5-A8h, or any preceding aspect, wherein the oligosaccharide composition comprises a combination of: (a) at least two (e.g., at least 3, 4, 5, 6, 7, 8, or 9; optionally less than 10, 9, 8, 7, 6, 5, 4, or 3; optionally wherein any of such values can be combined in any manner to form a range, such as 2-9) different isomers of 3Hex, (b) at least three (e.g., at least 3, 4, 5, 6, 7, 8, or 9; optionally less than 10, 9, 8, 7, 6, 5, or 4; optionally wherein any of such values can be combined in any manner to form a range, such as 3-7) different isomers of 3Hex, (c) at least four (e.g., at least 4, 5, 6, 7, 8, 9, 10, 11, 12, 13, 14, 15; optionally less than 16, 15, 14, 13, 12, 11, 10, 9, 8, 7, 6, or 5; optionally wherein any of such values can be combined in any manner to form a range, such as 8-16) different isomers of 4Hex, (d) at least five (e.g., at least 5, 6, 7, 8, 9, 10, 11, 12, 13, 14, 15; optionally less than 16, 15, 14, 13, 12, 11, 10, 9, 8, 7, or 6; optionally wherein any of such values can be combined in any manner to form a range, such as 5-12) different isomers of 5Hex, and (e) at least three (e.g., at least 3, 4, 5, 6, 7, 8, or 9; optionally less than 10, 9, 8, 7, 6, 5, or 4; optionally wherein any of such values can be combined in any manner to form a range, such as 3-7) different isomers of 6Hex.

Aspect A9: The method of any one of aspects A5-A8, or any preceding aspect, wherein the oligosaccharide composition is derived from a material comprising Alcaligenes faecalis, konjac, locust bean, Icelandic moss, carob, barley, tamarind, oat, or any combination thereof.

Aspect A10: The method of any one of aspects A5-A9, or any preceding aspect, wherein the oligosaccharide composition is derived from a material comprising curdlan, glucomannan, galactomannan, lichenin, cereal beta glucan, xyloglucan, or any combination thereof.

Aspect A11: The method of any one of aspects A5-A10, or any preceding aspect, wherein the oligosaccharide composition comprises: CLX101, CLX102, CLX108, CLX110, CLX111, CLX112, CLX113, CLX115a, CLX115-FC, CLX155-AL, or any combination thereof.

Aspect A12: The method of any one of aspects A5-A11, or any preceding aspect, wherein the microbiota comprise:
Clostridium cluster I, optionally selected from Clostridium butyricum;
Clostridium cluster IV, optionally selected from Faecalibacterium prausnitzii;
Clostridium cluster XIVa, optionally selected from Roseburia spp, Eubacterium rectale, Eubacterium hallii, Blautia, Ruminococcus, Dorea, Coprococcus, or any combination thereof; or
any combination thereof.

Aspect A13: The method of any one of aspects A5-A12, or any preceding aspect, wherein the method modulates the microbiota to produce the at least one short chain fatty acid, wherein the at least one short chain fatty acid comprises butyrate.

Aspect A14: The method of any one of aspects A1-A4, or any preceding aspect, wherein the oligosaccharide composition comprises the at least one second feature.

Aspect A15: The method of aspect A14, or any preceding aspect, wherein the oligosaccharide composition comprises:
a sum of rhamnose, galacturonic acid, arabinose, fucose, and mannose subunits in an amount of at least 5 wt. % (e.g., at least any of the following: 5, 7, 10, 12, 15, 20, 25, or 30 wt. %; optionally less than any of the following: 35, 30, 25, 20, 15, 12, 10, or 7 wt. %; optionally wherein any of such values can be combined in any manner to form a range, such as 5-25 wt. %), based on total weight of saccharide subunits; and
a sum of arabinose subunits in an amount of 20 wt. % to 90 wt. % (e.g., 20-30, 20-40, 20-50, 20-60, 20-70, 20-80, 30-40, 30-50, 30-60, 30-70, 30-80, 30-90, 40-50, 40-60, 40-70, 40-80, 40-90, 50-60, 50-70, 50-80, 50-90, 60-70, 60-80, 60-90, 70-80, 70-90, or 80-90 wt. %), based on total weight of saccharide subunits; and a sum of galactose subunits in an amount of 5 wt. % to 60 wt. % (e.g., 5-10, 5-20, 5-30, 5-40, 5-50, 10-20, 10-30, 10-40, 10-50, 10-60, 20-30, 20-40, 20-50, 20-60, 30-40, 30-50, 30-60, 40-50, 40-60, or 50-60 wt. %), based on total weight of saccharide subunits.

Aspect A16: The method of aspect A14 or A15, or any preceding aspect, wherein at least 70 wt. % (e.g., at least any of the following: 75, 80, 85, 90, 95, 97, or 99 wt. %; optionally essentially all; optionally 100 wt. %; optionally less than any of the following: 100, 99, 97, 95, 90, 85, 80, or 75 wt. %; optionally where any such values can be combined in any manner to form a range, such as 90-99 wt. % or 95 wt. % to essentially all) of the non-terminal glucose subunits are β1,3 linked, § 1,4 linked, and/or β1,6 linked.

Aspect A17: The method of any one of aspects A14-A16, or any preceding aspect, wherein the oligosaccharide composition comprises non-terminal galactose subunits, wherein at least 70 wt. % (e.g., at least any of the following: 75, 80, 85, 90, 95, 97, or 99 wt. %; optionally essentially all; optionally 100 wt. %; optionally less than any of the following: 100, 99, 97, 95, 90, 85, 80, or 75 wt. %; optionally where any such values can be combined in any manner to form a range, such as 85-99 wt. % or 90 wt. % to essentially all) of the non-terminal galactose subunits have at least one 4-linkage.

Aspect A18: The method of aspect A17, or any preceding aspect, wherein at least 70 wt. % (e.g., at least any of the following: 75, 80, 85, 90, 95, 97, or 99 wt. %; optionally essentially all; optionally 100 wt. %; optionally less than any of the following: 100, 99, 97, 95, 90, 85, 80, or 75 wt. %; optionally where any such values can be combined in any manner to form a range, such as 75-90 wt. % or 99 wt. % to essentially all) of the non-terminal galactose subunits are β1,4 linked.

Aspect A19: The method of any one of aspects A14-A18, or any preceding aspect, wherein the oligosaccharide composition comprises non-terminal arabinose subunits, wherein at least 70 wt. % (e.g., at least any of the following: 75, 80, 85, 90, 95, 97, or 99 wt. %; optionally essentially all; optionally 100 wt. %; optionally less than any of the following: 100, 99, 97, 95, 90, 85, 80, or 75 wt. %; optionally where any such values can be combined in any manner to form a range, such as 85-99 wt. % or 90 wt. % to essentially all) of the non-terminal arabinose subunits have at least one 5-linkage.

Aspect A20: The method of aspect A19, or any preceding aspect, wherein at least 70 wt. % (e.g., at least any of the following: 75, 80, 85, 90, 95, 97, or 99 wt. %; optionally essentially all; optionally 100 wt. %; optionally less than any of the following: 100, 99, 97, 95, 90, 85, 80, or 75 wt. %; optionally where any such values can be combined in any manner to form a range, such as 80-97 wt. % or 97 wt. % to essentially all) of the non-terminal arabinose subunits have at least one α1,5 linkage.

Aspect A21: The method of any one of aspects A14-A19, or any preceding aspect, wherein the oligosaccharide composition comprises:
a sum of mannose subunits in an amount of at least 50 wt. % (e.g., at least any of the following: 50, 55, 60, 65, 70, 75, 80, 85, 90, 95, or 99 wt. %; optionally 100 wt. %; optionally less than any of the following: 100, 99, 95, 90, 85, 80, 75, 70, 65, 60, or 55 wt. %; optionally wherein any of such values can be combined in any manner to form a range, such as 75-95 wt. %), based on total weight of saccharide subunits; and a sum of galactose subunits in an amount of less than 20 wt. %, based on total weight of saccharide subunits; and
a sum of arabinose subunits in an amount of 0 wt. % to 50 wt. %, based on total weight of saccharide subunits.

Aspect A22: The method of any one of aspects A14-A21, or any preceding aspect, wherein the oligosaccharide composition comprises:
non-terminal xylose subunits, wherein at least 70 wt. % (e.g., at least any of the following: 75, 80, 85, 90, 95, 97, or 99 wt. %; optionally essentially all; optionally 100 wt. %; optionally less than any of the following: 100, 99, 97, 95, 90, 85, 80, or 75 wt. %; optionally where any such values can be combined in any manner to form a range, such as 85-99 wt. % or 97 wt. % to essentially all) of the non-terminal xylose subunits have at least one 4-linkage; and
arabinose subunits, wherein at least 70 wt. % (e.g., at least any of the following: 75, 80, 85, 90, 95, 97, or 99 wt. %; optionally essentially all; optionally 100 wt. %; optionally less than any of the following: 100, 99, 97, 95, 90, 85, 80, or 75 wt. %; optionally where any such values can be combined in any manner to form a range, such as 85-99 wt. % or 90 wt. % to essentially all) of the arabinose subunits are terminal arabinose subunits.

Aspect A23: The method of aspect A22, or any preceding aspect, wherein at least 70 wt. % (e.g., at least any of the following: 75, 80, 85, 90, 95, 97, or 99 wt. %; optionally essentially all; optionally 100 wt. %; optionally less than any of the following: 100, 99, 97, 95, 90, 85, 80, or 75 wt. %; optionally where any such values can be combined in any manner to form a range, such as 95-99 wt. % or 95 wt. % to essentially all) of the non-terminal xylose subunits are β1-4 linked.

Aspect A24: The method of any one of aspects A14-A23, or any preceding aspect, wherein the oligosaccharide composition is derived from a material comprising beechwood, larch, potato, carob, rye, wheat, corn, Sterculia, Saccharomyces Cerevisiae, Astragalus, pea, Xanthamonas campestris, coffee, sphingomonas elodea, soy, carrot, sugarcane, kelp, sea lettuce, olive, or any combination thereof.

Aspect A25: The method of any one of aspects A14-A24, or any preceding aspect, wherein the oligosaccharide composition is derived from a material comprising xylan, arabinogalactan, galactan, galactomannan, arabinoxylan, karaya gum, yeast mannan, yeast beta glucan, tragacanth gum, arabinan, xantham gum, galactan, mannan, gellen gum, pectin, fucoidan, or any combination thereof.

Aspect A26: The method of any one of aspects A14-A25, or any preceding aspect, wherein the oligosaccharide composition comprises CLX103, CLX105, CLX109, CLX111, CLX114, CLX107, CLX115a, CLX117, CLX122, CLX122-DS, CLX122-DSF, CLX123, CLX124, CLX125, CLX126, CLX127, CLX128, CLX129, CLX130, CLX131, or any combination thereof.

Aspect A27: The method of any one of aspects A14-A26, or any preceding aspect, wherein the microbiota comprise: Bacteroides thetaiotaomicron, Bacteroides ovatus, Bacteroides fragilis, Akkermansia Mucinophila, Bacteroides eggerthii, Ruminococcus bromii, Eubacterium dolichum, Veillonella parvula, or any combination thereof.

Aspect A28: The method of any one of aspects A14-A27, or any preceding aspect, wherein the method modulates the microbiota to produce the at least one short chain fatty acid, wherein the at least one short chain fatty acid comprises propionate.

Aspect A29: The method of any one of aspects A1-A4, or any preceding aspect, wherein the oligosaccharide composition comprises the at least one third feature.

Aspect A30: The method of aspect A29, or any preceding aspect, wherein the oligosaccharide composition comprises:
a sum of glucose, galactose, mannose subunits in an amount of at least 50 wt. % (e.g., at least any of the following: 50, 55, 60, 65, 70, 75, 80, 85, 90, 95, or 99 wt. %; optionally 100 wt. %; optionally less than any of the following: 100, 99, 95, 90, 85, 80, 75, 70, 65, 60, or 55 wt. %; optionally wherein any of such values can be combined in any manner to form a range, such as 75-99 wt. %), based on total weight of saccharide subunits; and
a sum of arabinose subunits in an amount of less than 8 wt. % (e.g., less than any of the following: 8, 7, 6, 5, 4, 3, 2, 1, or 0.5 wt. %; optionally at least any of the following: 0.1, 0.5, 1, 2, 3, 4, 5, 6, or 7 wt. %; optionally wherein any of such values can be combined in any manner to form a range, such as 3-7 wt. %), based on total weight of saccharide subunits.

Aspect A31: The method of aspect A29 or A30, or any preceding aspect, wherein the oligosaccharide composition comprises less than 15 wt. % (e.g., less than any of the following: 15, 12, 10, 7, 5, or 3 wt. %; optionally at least any of the following: 1, 3, 5, 7, 10, or 12 wt. %; optionally wherein any of such values can be combined in any manner to form a range, such as 7-10 wt. %) 2-linked mannose subunits, based on total weight of saccharide subunits.

Aspect A32: The method of aspect A31, or any preceding aspect, wherein the 2-linked mannose subunits are α1,2 linked.

Aspect A33: The method of any one of aspects A29-A32, or any preceding aspect, wherein the oligosaccharide composition comprises:
a sum of glucose subunits in an amount of at least 20 wt. % (e.g., at least any of the following: 22, 24, 26, 28, 30, 32, 35, 37, 40, 42, 45, or 47 wt. %; optionally less than any of the following: 50, 47, 45, 42, 40, 37, 35, 32, 30, 28, 26, or 24 wt. %; optionally wherein any of such values can be combined in any manner to form a range, such as 30-50 wt. %), based on total weight of saccharide subunits; and a sum of xylose and arabinose subunits in an amount of at least 33 wt. % (e.g., at least any of the following: 35, 40, 45, 50, 55, 60, 65, 70, 75, 80, 85, or 90 wt. %; optionally less than any of the following: 95, 90, 85, 80, 75, 70, 65, 60, 55, 50, 45, 40, or 35 wt. %; optionally wherein any of such values can be combined in any manner to form a range, such as 33-55 wt. %), based on total weight of saccharide subunits; and
non-terminal glucose subunits, wherein at least 70 wt. % (e.g., at least any of the following: 75, 80, 85, 90, 95, 97, or 99 wt. %; optionally essentially all; optionally 100 wt. %; optionally less than any of the following: 100, 99, 97, 95, 90, 85, 80, or 75 wt. %; optionally where any such values can be combined in any manner to form a range, such as 85-99 wt. % or 90 wt. % to essentially all) of the non-terminal glucose subunits have at least one 3-linkage and/or at least one 4-linkage.

Aspect A34: The method of aspect A33, or any preceding aspect, wherein at least 70 wt. % (e.g., at least any of the following: 75, 80, 85, 90, 95, 97, or 99 wt. %; optionally essentially all; optionally 100 wt. %; optionally less than any of the following: 100, 99, 97, 95, 90, 85, 80, or 75 wt. %; optionally where any such values can be combined in any manner to form a range, such as 85-99 wt. % or 90 wt. % to essentially all) of the non-terminal glucose subunits are β1,3 linked and/or β1,4 linked.

Aspect A35: The method of any one of aspects A29-A34, or any preceding aspect, wherein the oligosaccharide composition comprises:
non-terminal xylose subunits, wherein at least 70 wt. % (e.g., at least any of the following: 75, 80, 85, 90, 95, 97, or 99 wt. %; optionally essentially all; optionally 100 wt. %; optionally less than any of the following: 100, 99, 97, 95, 90, 85, 80, or 75 wt. %; optionally where any such values can be combined in any manner to form a range, such as 85-99 wt. % or 90 wt. % to essentially all) the non-terminal xylose subunits have at least one 4-linkage; and
arabinose subunits, wherein at least 70 wt. % (e.g., at least any of the following: 75, 80, 85, 90, 95, 97, or 99 wt. %; optionally essentially all; optionally 100 wt. %; optionally less than any of the following: 100, 99, 97, 95, 90, 85, 80, or 75 wt. %; optionally where any such values can be combined in any manner to form a range, such as 85-99 wt. % or 90 wt. % to essentially all) of the arabinose subunits are terminal arabinose subunits.

Aspect A36a: The method of aspect A35, or any preceding aspect, wherein at least 70 wt. % (e.g., at least any of the following: 75, 80, 85, 90, 95, 97, or 99 wt. %; optionally essentially all; optionally 100 wt. %; optionally less than any of the following: 100, 99, 97, 95, 90, 85, 80, or 75 wt. %; optionally where any such values can be combined in any manner to form a range, such as 85-99 wt. % or 90 wt. % to essentially all) the non-terminal xylose subunits have β1-4 linkages.

Aspect A36b: The method of any one of aspects A29-A36a, or any preceding aspect, wherein the oligosaccharide composition comprises non-terminal glucose subunits, wherein (1) 20 wt. % to 100 wt. % (e.g., 20-30, 20-40, 20-50, 20-60, 20-70, 20-80, 20-90, 20-95, 20-99, 30-40, 30-50, 30-60, 30-70, 30-80, 30-90, 30-95, 30-99, 30-100, 40-50, 40-60, 40-70, 40-80, 40-90, 40-95, 40-99, 40-100, 50-60, 50-70, 50-80, 50-90, 50-95, 50-99, 50-100, 60-70, 60-80, 60-90, 60-95, 60-99, 60-100, 70-80, 70-90, 70-95, 70-99, 70-100, 80-90, 80-95, 80-99, 80-100, 90-95, 90-99, 90-100, 95-99, 95-100, or 99-100 wt. %) of the non-terminal glucose subunits have at least one 3-linkage, based on total weight of non-terminal saccharide subunits; and/or (2) 33 wt. % to 80 wt. % (e.g., 33-50, 33-60, 33-70, 40-50, 40-60, 40-70, 40-80, 50-60, 50-70, 50-80, 60-70, 60-80, or 70-80 wt. %) of the non-terminal glucose subunits have at least one 4-linkage, based on total weight of non-terminal saccharide subunits.

Aspect A36c: The method of any one of aspects A29-A36b, or any preceding aspect, wherein the oligosaccharide composition comprises non-terminal glucose subunits, wherein (1) 20 wt. % to 100 wt. % (e.g., 20-30, 20-40, 20-50, 20-60, 20-70, 20-80, 20-90, 20-95, 20-99, 30-40, 30-50, 30-60, 30-70, 30-80, 30-90, 30-95, 30-99, 30-100, 40-50, 40-60, 40-70, 40-80, 40-90, 40-95, 40-99, 40-100, 50-60, 50-70, 50-80, 50-90, 50-95, 50-99, 50-100, 60-70, 60-80, 60-90, 60-95, 60-99, 60-100, 70-80, 70-90, 70-95, 70-99, 70-100, 80-90, 80-95, 80-99, 80-100, 90-95, 90-99, 90-100, 95-99, 95-100, or 99-100 wt. %) of the non-terminal glucose subunits are β1-3 linked, based on total weight of non-terminal saccharide subunits; and/or (2) 33 wt. % to 80 wt. % (e.g., 33-50, 33-60, 33-70, 40-50, 40-60, 40-70, 40-80, 50-60, 50-70, 50-80, 60-70, 60-80, or 70-80 wt. %) of the non-terminal glucose subunits are β1-4 linked, based on total weight of non-terminal saccharide subunits.

Aspect A36d: The method of any one of aspects A29-A36c, or any preceding aspect, wherein the oligosaccharide composition comprises:
non-terminal glucose subunits, wherein (1) 20 wt. % to 100 wt. % (e.g., 20-30, 20-40, 20-50, 20-60, 20-70, 20-80, 20-90, 20-95, 20-99, 30-40, 30-50, 30-60, 30-70, 30-80, 30-90, 30-95, 30-99, 30-100, 40-50, 40-60, 40-70, 40-80, 40-90, 40-95, 40-99, 40-100, 50-60, 50-70, 50-80, 50-90, 50-95, 50-99, 50-100, 60-70, 60-80, 60-90, 60-95, 60-99, 60-100, 70-80, 70-90, 70-95, 70-99, 70-100, 80-90, 80-95, 80-99, 80-100, 90-95, 90-99, 90-100, 95-99, 95-100, or 99-100 wt. %) of the non-terminal glucose subunits have at least one 3-linkage, based on total weight of non-terminal saccharide subunits; and/or (2) 33 wt. % to 80 wt. % (e.g., 33-50, 33-60, 33-70, 40-50, 40-60, 40-70, 40-80, 50-60, 50-70, 50-80, 60-70, 60-80, or 70-80 wt. %) of the non-terminal glucose subunits have at least one 4-linkage, based on total weight of non-terminal saccharide subunits; and
a weight ratio of glucose subunits having at least one 4-linkage to glucose subunits having at least one 3-linkage is between 2:1 to 4:1 (e.g., 2:1 to 2.5:1, 2:1 to 3:1, 2:1 to 3.5:1, 2.5:1 to 3:1, 2.5:1 to 3.5:1, 2.5:1 to 4:1, 3:1 to 3.5:1, 3:1 to 4:1, or 3.5:1 to 4:1).

Aspect A36e: The method of any one of aspects A29-A36d, or any preceding aspect, wherein the oligosaccharide composition comprises a weight ratio of glucose subunits having at least one 4-linkage to glucose subunits having at least one 3-linkage of between 2:1 to 4:1 (e.g., 2:1 to 2.5:1, 2:1 to 3:1, 2:1 to 3.5:1, 2.5:1 to 3:1, 2.5:1 to 3.5:1, 2.5:1 to 4:1, 3:1 to 3.5:1, 3:1 to 4:1, or 3.5:1 to 4:1).

Aspect A36f: The method of any one of aspects A29-A36e, or any preceding aspect, wherein the oligosaccharide composition comprises a weight ratio of glucose subunits having β1-4 linkages to glucose subunits having P1-3 linkages of between 2:1 to 4:1 (e.g., 2:1 to 2.5:1, 2:1 to 3:1, 2:1 to 3.5:1, 2.5:1 to 3:1, 2.5:1 to 3.5:1, 2.5:1 to 4:1, 3:1 to 3.5:1, 3:1 to 4:1, or 3.5:1 to 4:1).

Aspect A36g: The method of any one of aspects A29-A36f, or any preceding aspect, wherein the oligosaccharide composition comprises non-terminal glucose subunits, wherein 30 wt. % to 45 wt. % (e.g., 30-32, 30-35, 30-37, 30-40, 30-42, 32-35, 32-37, 32-40, 32-42, 32-45, 35-37, 35-40, 35-42, 35-45, 37-40, 37-42, 37-45, 40-42, 40-45, or 42-45 wt. %) of the non-terminal glucose subunits are $31-3 linked, based on total weight of non-terminal saccharide subunits.

Aspect A36h: The method of any one of aspects A29-A36g, or any preceding aspect, wherein the oligosaccharide composition comprises at least one of (e.g., at least two of, at least three of, at least four of, at least five of, at least six of, at least seven of, or at least eight of): (a) at least two (e.g., at least 3, 4, 5, 6, 7, 8, or 9; optionally less than 10, 9, 8, 7, 6, 5, 4, or 3; optionally wherein any of such values can be combined in any manner to form a range, such as 2-9) different isomers of 3Hex, (b) at least three (e.g., at least 3, 4, 5, 6, 7, 8, or 9; optionally less than 10, 9, 8, 7, 6, 5, or 4; optionally wherein any of such values can be combined in any manner to form a range, such as 3-7) different isomers of 3Hex, (c) at least four (e.g., at least 4, 5, 6, 7, 8, 9, 10, 11, 12, 13, 14, 15; optionally less than 16, 15, 14, 13, 12, 11, 10, 9, 8, 7, 6, or 5; optionally wherein any of such values can be combined in any manner to form a range, such as 8-16) different isomers of 4Hex, (d) at least five (e.g., at least 5, 6, 7, 8, 9, 10, 11, 12, 13, 14, 15; optionally less than 16, 15, 14, 13, 12, 11, 10, 9, 8, 7, or 6; optionally wherein any of such values can be combined in any manner to form a range, such as 5-12) different isomers of 5Hex, (e) at least three (e.g., at least 3, 4, 5, 6, 7, 8, or 9; optionally less than 10, 9, 8, 7, 6, 5, or 4; optionally wherein any of such values can be combined in any manner to form a range, such as 3-7) different isomers of 6Hex, or (f) any combination thereof.

Aspect A36i: The method of any one of aspects A29-A36h, or any preceding aspect, wherein the oligosaccharide composition comprises a combination of: (a) at least two (e.g., at least 3, 4, 5, 6, 7, 8, or 9; optionally less than 10, 9, 8, 7, 6, 5, 4, or 3; optionally wherein any of such values can be combined in any manner to form a range, such as 2-9) different isomers of 3Hex, (b) at least three (e.g., at least 3, 4, 5, 6, 7, 8, or 9; optionally less than 10, 9, 8, 7, 6, 5, or 4; optionally wherein any of such values can be combined in any manner to form a range, such as 3-7) different isomers of 3Hex, (c) at least four (e.g., at least 4, 5, 6, 7, 8, 9, 10, 11, 12, 13, 14, 15; optionally less than 16, 15, 14, 13, 12, 11, 10, 9, 8, 7, 6, or 5; optionally wherein any of such values can be combined in any manner to form a range, such as 8-16) different isomers of 4Hex, (d) at least five (e.g., at least 5, 6, 7, 8, 9, 10, 11, 12, 13, 14, 15; optionally less than 16, 15, 14, 13, 12, 11, 10, 9, 8, 7, or 6; optionally wherein any of such values can be combined in any manner to form a range, such as 5-12) different isomers of 5Hex, and (e) at least three (e.g., at least 3, 4, 5, 6, 7, 8, or 9; optionally less than 10, 9, 8, 7, 6, 5, or 4; optionally wherein any of such values can be combined in any manner to form a range, such as 3-7) different isomers of 6Hex.

Aspect A37: The method of any one of aspects A29-A36, or any preceding aspect, wherein the oligosaccharide composition is derived from a material comprising Alcaligenes faecalis, konjac, locust bean, Icelandic moss, carob, barley, oat, sugar cane, or any combination thereof.

Aspect A38: The method of any one of aspects A29-A37, or any preceding aspect, wherein the oligosaccharide composition is derived from a material comprising curdlan, glucomannan, galactomannan, lichenin, cereal beta glucan, sugar cane, or any combination thereof.

Aspect A39: The method of any one of aspects A29-A38, or any preceding aspect, wherein the oligosaccharide composition comprises CLX101, CLX102, CLX108, CLX110, CLX111, CLX112, CLX115, CLX115-AL, CLX115-FC, CLX128, or any combination thereof.

Aspect A40: The method of any one of aspects A29-A39, or any preceding aspect, wherein the microbiota comprise Clostridium cluster I, optionally selected from Clostridium butyricum.

Aspect A41: The method of any one of aspects A29-A40, or any preceding aspect, wherein the method modulates the microbiota to produce the at least one short chain fatty acid, wherein the at least one short chain fatty acid comprises betahydroxybutyrate.

Aspect A42: The method of any one of aspects A1-A41, or any preceding aspect, wherein the method increases abundance of the microbiota.

Aspect A43: The method of any one of aspects A1-A42, or any preceding aspect, wherein the method modulates the microbiota to produce the at least one short chain fatty acid.

Aspect A44: The method of any one of aspects A1-A43, or any preceding aspect, wherein the at least one short chain fatty acid comprises butyrate, propionate, betahydroxybutyrate, lactate, acetate, or any combination thereof.

Aspect A45: The method of any one of aspects A1-A44, or any preceding aspect, wherein the method causes the microbiota to produce an increased amount of the at least one short chain fatty acid compared to an otherwise identical method that excludes contacting the microbiota with the formulation comprising the oligosaccharide composition.

Aspect A46: The method of any one of aspects A1-A45, or any preceding aspect, wherein the microbiota are located in a gastrointestinal tract of a subject, and the contacting step comprises administering the formulation to the subject.

Aspect A47: The method of aspect A46, or any preceding aspect, wherein the subject is in need of the formulation to prevent or treat a disease, condition, disorder, and/or indication, and the contacting step comprises administering to the subject a therapeutically effective amount of the formulation.

Aspect A48: The method of aspect A46 or A47, or any preceding aspect, wherein the method increases production of the at least one short chain fatty acid in the gastrointestinal tract of the subject.

Aspect A49: The method of any one of aspects A46-A48, or any preceding aspect, wherein the method lowers inflammation in the gastrointestinal tract of the subject.

Aspect A50: The method of any one of aspects A46-A49, or any preceding aspect, wherein the method lowers at least one of inflammatory gastrointestinal markers TNF-α, IL1-β, or IL-6, or any combination thereof, in the subject.

Aspect A51: The method of any one of aspects A46-A50, or any preceding aspect, wherein the method increases at least one of anti-inflammatory gastrointestinal markers IL-10, TGF-beta, or IL-4, or any combination thereof, in the subject.

Aspect A52: The method of any one of aspects A46-A51, or any preceding aspect, wherein decreases intestinal barrier permeability in the gastrointestinal tract of the subject.

Aspect A53: The method of any one of aspects A46-A52, or any preceding aspect, wherein the method increases the expression of Muc2, occluding, claudin-4, or ZO-1 genes, or any combination thereof, in the gastrointestinal tract of the subject.

Aspect A54: The method of any one of aspects A46-A53, or any preceding aspect, wherein the method lowers A1c levels in the subject.

Aspect A55: The method of any one of aspects A46-A54, or any preceding aspect, wherein the disease, condition, disorder, and/or indication is type 2 diabetes mellitus, atherosclerosis, IBD, Crohn's, NAFLD, Constipation, Celiac Disease, Diarrhea, IBS, or any combination thereof.

Aspect A56: The method of any one of aspects A46-A55, or any preceding aspect, wherein the oligosaccharide composition comprises the at least one first feature, and the method stimulates at least one of receptors GPR41, GPR43, GPR109A, or AhR, or any combination thereof, in the subject.

Aspect A57: The method of any one of aspects A46-A56, or any preceding aspect, wherein the oligosaccharide composition comprises the at least one first feature, and the method increases the production of GLP-1 in the subject.

Aspect A58: The method of any one of aspects A46-A57, or any preceding aspect, wherein the oligosaccharide composition comprises the at least one first feature, and the method inhibits histone deacetylases in the subject.

Aspect A59: The method of any one of aspects A46-A58, or any preceding aspect, wherein the oligosaccharide composition comprises the at least one first feature, and the method promotes gastrointestinal motility in the subject.

Aspect A60: The method of any one of aspects A46-A59, or any preceding aspect, wherein the oligosaccharide composition comprises the at least one first feature, and the method lowers post-prandial glucose response in the subject.

Aspect A61: The method of any one of aspects A46-A60, or any preceding aspect, wherein the oligosaccharide composition comprises the at least one first feature, and the disease, condition, disorder, and/or indication is visceral pain.

Aspect A62: The method of any one of aspects A46-A55, or any preceding aspect, wherein the oligosaccharide composition comprises the at least one second feature, and the method stimulates at least one of receptors GPR41, GPR43, or PSGR, or any combination thereof, in the subject.

Aspect A63: The method of any one of aspects A46-A55 or A62, or any preceding aspect, wherein the method increases the production of GLP-1 in the subject.

Aspect A64: The method of any one of aspects A46-A55, or any preceding aspect, wherein the method inhibits histone deacetylases in the subject.

Aspect A65: The method of any one of aspects A46-A55 or A64, or any preceding aspect, wherein the disease, condition, disorder, and/or indication is epilepsy.

Aspect A66: A method for modulating microbiota to: produce at least one indole derivative, metabolize bile acid and/or bile salt, increase an abundance of the microbiota, or any combination thereof; the method comprising:
contacting the microbiota with a formulation comprising an oligosaccharide composition;
wherein the method modulates the microbiota to: produce the at least one indole derivative, metabolize the bile acids and/or bile salts, increase the abundance of the microbiota, or any combination thereof; and
wherein the oligosaccharide composition comprises:
   a sum of galactose subunits in an amount of 3 wt. % to 60 wt. % (e.g., 3-10, 3-20, 3-30, 3-40, 3-50, 5-10, 5-20, 5-30, 5-40, 5-50, 5-60, 10-20, 10-30, 10-40, 10-50, 10-60, 20-30, 20-40, 20-50, 20-60, 30-40, 30-50, 30-60, 40-50, 40-60, or 50-60 wt. %) galactose, based on total weight of saccharide subunits; and a sum of arabinose subunits in an amount of 20 wt. % to 90 wt. % (e.g., 20-30, 20-40, 20-50, 20-60, 20-70, 20-80, 30-40, 30-50, 30-60, 30-70, 30-80, 30-90, 40-50, 40-60, 40-70, 40-80, 40-90, 50-60, 50-70, 50-80, 50-90, 60-70, 60-80, 60-90, 70-80, 70-90, or 80-90 wt. %), based on total weight of saccharide subunits;
   non-terminal glucose subunits, wherein (1) 20 wt. % to 100 wt. % (e.g., 20-30, 20-40, 20-50, 20-60, 20-70, 20-80, 20-90, 20-95, 20-99, 30-40, 30-50, 30-60, 30-70, 30-80, 30-90, 30-95, 30-99, 30-100, 40-50, 40-60, 40-70, 40-80, 40-90, 40-95, 40-99, 40-100, 50-60, 50-70, 50-80, 50-90, 50-95, 50-99, 50-100, 60-70, 60-80, 60-90, 60-95, 60-99, 60-100, 70-80, 70-90, 70-95, 70-99, 70-100, 80-90, 80-95, 80-99, 80-100, 90-95, 90-99, 90-100, 95-99, 95-100, or 99-100 wt. %) of the non-terminal glucose subunits have at least one 3-linkage, based on total weight of non-terminal saccharide subunits; and/or (2) 33 wt. % to 80 wt. % (e.g., 33-50, 33-60, 33-70, 40-50, 40-60, 40-70, 40-80, 50-60, 50-70, 50-80, 60-70, 60-80, or 70-80 wt. %) of the non-terminal glucose subunits have at least one 4-linkage, based on total weight of non-terminal saccharide subunits;
   a sum of mannose subunits in an amount of 33 wt. % to 70 wt. % (e.g., 33-50, 33-60, 40-50, 40-60, 40-70, 50-60, 50-70, or wt. %), based on total weight of saccharide subunits; and a sum of glucose subunits in an amount of 18 wt. % to 45 wt. % (e.g., 18-25, 18-30, 18-35, 18-40, 25-30, 25-35, 25-40, 25-45, 30-35, 30-40, 30-45, 35-40, 35-45, or 40-45 wt. %), based on total weight of saccharide subunits;
   a sum of xylose subunits in an amount of 33 wt. % to 70 wt. % (e.g., 33-40, 33-50, 33-60, 40-50, 40-60, 40-70, 50-60, 50-70, or wt. %), based on total weight of saccharide subunits; and a sum of arabinose subunits in an amount of 18 wt. % to 45 wt. % (e.g., 18-25, 18-30, 18-35, 18-40, 25-30, 25-35, 25-40, 25-45, 30-35, 30-40, 30-45, 35-40, 35-45, or 40-45 wt. %), based on total weight of saccharide subunits; or
   any combination thereof.

Aspect A67: The method of aspect A66, or any preceding aspect, wherein the oligosaccharide composition comprises at least 50 wt. % (e.g., at least any of the following: 50, 55, 60, 65, 70, 75, 80, 85, 90, 95, or 99 wt. %; optionally 100 wt. %; optionally less than any of the following: 100, 99, 95, 90, 85, 80, 75, 70, 65, 60, or 55 wt. %; optionally wherein any of such values can be combined in any manner to form a range, such as 55-90 wt. %) oligosaccharides on a dry basis having a degree of polymerization of between 3 and 50 (e.g., 3-5, 3-8, 3-10, 3-12, 3-15, 3-18, 3-20, 3-25, 3-30, 3-35, 3-40, 3-45, 5-8, 5-10, 5-12, 5-15, 5-18, 5-20, 5-25, 5-30, 5-35, 5-40, 5-45, 5-50, 10-15, 10-20, 10-25, 10-30, 10-35, 10-40, 10-45, 10-50, 15-20, 15-25, 15-30, 15-35, 15-40, 15-45, 15-50, 20-25, 20-30, 20-35, 20-40, 20-45, 20-50, 25-30, 25-35, 25-40, 25-45, 25-50, 30-35, 30-40, 30-45, 30-50, 35-40, 35-45, 35-50, 40-45, 40-50, or 45-50) monosaccharide subunits.

Aspect A68: The method of Aspect A66 or A67, or any preceding aspect, wherein the oligosaccharide composition comprises a dynamic viscosity at 25° C. of between 0.8 and 2.5 (e.g., 0.8-1, 0.8-1.2, 0.8-1.5, 0.8-1.7, 0.8-2, 0.8-2.2, 1-1.2, 1-1.5, 1-1.7, 1-2, 1-2.2, 1-2.5, 1.2-1.5, 1.2-1.7, 1.2-2, 1.2-2.2, 1.2-2.5, 1.5-1.7, 1.5-2, 1.5-2.2, 1.5-2.5, 1.7-2, 1.7-2.2, 1.7-2.5, 2-2.2, 2-2.5, or 2.2-2.5) mPa*s at a concentration of 100 mg of the oligosaccharide composition on a dry basis in 1 mL of water.

Aspect A69: The method of any one of aspects A66-A68, or any preceding aspect, wherein the oligosaccharide composition comprises less than 5 wt. % (e.g., less than any of the following: 4, 3, 2, 1, or 0.5 wt. %; optionally at least any of the following: 0.1, 0.5, 1, 2, 3, or 4 wt. %; optionally wherein any of such values can be combined in any manner to form a range, such as 0.1-3 wt. %) monosaccharides.

Aspect A70: The method of any one of aspects A66-A69, or any preceding aspect, wherein the oligosaccharide composition comprises non-terminal glucose subunits, wherein (1) 20 wt. % to 100 wt. % (e.g., 20-30, 20-40, 20-50, 20-60, 20-70, 20-80, 20-90, 20-95, 20-99, 30-40, 30-50, 30-60, 30-70, 30-80, 30-90, 30-95, 30-99, 30-100, 40-50, 40-60, 40-70, 40-80, 40-90, 40-95, 40-99, 40-100, 50-60, 50-70, 50-80, 50-90, 50-95, 50-99, 50-100, 60-70, 60-80, 60-90, 60-95, 60-99, 60-100, 70-80, 70-90, 70-95, 70-99, 70-100, 80-90, 80-95, 80-99, 80-100, 90-95, 90-99, 90-100, 95-99, 95-100, or 99-100 wt. %) of the non-terminal glucose subunits are β1-3 linked, based on total weight of non-terminal saccharide subunits; and/or (2) 33 wt. % to 80 wt. % (e.g., 33-50, 33-60, 33-70, 40-50, 40-60, 40-70, 40-80, 50-60, 50-70, 50-80, 60-70, 60-80, or 70-80 wt. %) of the non-terminal glucose subunits are β1-4 linked, based on total weight of non-terminal saccharide subunits.

Aspect A71: The method of any one of aspects A66-A70, or any preceding aspect, wherein the oligosaccharide composition comprises:
a sum of galactose subunits in an amount of 3 wt. % to 60 wt. % (e.g., 3-10, 3-20, 3-30, 3-40, 3-50, 5-10, 5-20, 5-30, 5-40, 5-50, 5-60, 10-20, 10-30, 10-40, 10-50, 10-60, 20-30, 20-40, 20-50, 20-60, 30-40, 30-50, 30-60, 40-50, 40-60, or 50-60 wt. %), based on total weight of saccharide subunits; and a sum of arabinose subunits in an amount of 20 wt. % to 90 wt. % (e.g., 20-30, 20-40, 20-50, 20-60, 20-70, 20-80, 30-40, 30-50, 30-60, 30-70, 30-80, 30-90, 40-50, 40-60, 40-70, 40-80, 40-90, 50-60, 50-70, 50-80, 50-90, 60-70, 60-80, 60-90, 70-80, 70-90, or 80-90 wt. %), based on total weight of saccharide subunits; and
non-terminal arabinose subunits, wherein at least 70 wt. % (e.g., at least any of the following: 75, 80, 85, 90, 95, 97, or 99 wt. %; optionally essentially all; optionally 100 wt. %; optionally less than any of the following: 100, 99, 97, 95, 90, 85, 80, or 75 wt. %; optionally where any such values can be combined in any manner to form a range, such as 85-99 wt. % or 90 wt. % to essentially all) of the non-terminal arabinose subunits have at least one 5-linkage.

Aspect A72: The method of aspect A71, or any preceding aspect, wherein at least 70 wt. % (e.g., at least any of the following: 75, 80, 85, 90, 95, 97, or 99 wt. %; optionally essentially all; optionally 100 wt. %; optionally less than any of the following: 100, 99, 97, 95, 90, 85, 80, or 75 wt. %; optionally where any such values can be combined in any manner to form a range, such as 85-99 wt. % or 90 wt. % to essentially all) of the non-terminal arabinose subunits have α1,5 linkages.

Aspect A73: The method of any one of aspects A66-A72, or any preceding aspect, wherein the oligosaccharide composition comprises non-terminal galactose subunits, wherein at least 70 wt. % (e.g., at least any of the following: 75, 80, 85, 90, 95, 97, or 99 wt. %; optionally essentially all; optionally 100 wt. %; optionally less than any of the following: 100, 99, 97, 95, 90, 85, 80, or 75 wt. %; optionally where any such values can be combined in any manner to form a range, such as 85-99 wt. % or 90 wt. % to essentially all) of the non-terminal galactose subunits have at least one 4-linkage.

Aspect A74: The method of aspect A73, or any preceding aspect, wherein at least 70 wt. % (e.g., at least any of the following: 75, 80, 85, 90, 95, 97, or 99 wt. %; optionally essentially all; optionally 100 wt. %; optionally less than any of the following: 100, 99, 97, 95, 90, 85, 80, or 75 wt. %; optionally where any such values can be combined in any manner to form a range, such as 85-99 wt. % or 90 wt. % to essentially all) of the non-terminal galactose subunits are β1,4 linked.

Aspect A75: The method of any one of aspects A66-A74, or any preceding aspect, wherein the oligosaccharide composition comprises:
non-terminal glucose subunits, wherein (1) 20 wt. % to 100 wt. % (e.g., 20-30, 20-40, 20-50, 20-60, 20-70, 20-80, 20-90, 20-95, 20-99, 30-40, 30-50, 30-60, 30-70, 30-80, 30-90, 30-95, 30-99, 30-100, 40-50, 40-60, 40-70, 40-80, 40-90, 40-95, 40-99, 40-100, 50-60, 50-70, 50-80, 50-90, 50-95, 50-99, 50-100, 60-70, 60-80, 60-90, 60-95, 60-99, 60-100, 70-80, 70-90, 70-95, 70-99, 70-100, 80-90, 80-95, 80-99, 80-100, 90-95, 90-99, 90-100, 95-99, 95-100, or 99-100 wt. %) of the non-terminal glucose subunits have at least one 3-linkage, based on total weight of non-terminal saccharide subunits; and/or (2) 33 wt. % to 80 wt. % (e.g., 33-50, 33-60, 33-70, 40-50, 40-60, 40-70, 40-80, 50-60, 50-70, 50-80, 60-70, 60-80, or 70-80 wt. %) of the non-terminal glucose subunits have at least one 4-linkage, based on total weight of non-terminal saccharide subunits; and
a weight ratio of glucose subunits having at least one 4-linkage to glucose subunits having at least one 3-linkage is between 2:1 to 4:1 (e.g., 2:1 to 2.5:1, 2:1 to 3:1, 2:1 to 3.5:1, 2.5:1 to 3:1, 2.5:1 to 3.5:1, 2.5:1 to 4:1, 3:1 to 3.5:1, 3:1 to 4:1, or 3.5:1 to 4:1).

Aspect A76: The method of any one of aspects A66-A75, or any preceding aspect, wherein a weight ratio of glucose subunits having at least one 4-linkage to glucose subunits having at least one 3-linkage is between 2:1 to 4:1 (e.g., 2:1 to 2.5:1, 2:1 to 3:1, 2:1 to 3.5:1, 2.5:1 to 3:1, 2.5:1 to 3.5:1, 2.5:1 to 4:1, 3:1 to 3.5:1, 3:1 to 4:1, or 3.5:1 to 4:1).

Aspect A77: The method of any one of aspects A66-A76, or any preceding aspect, wherein a weight ratio of glucose subunits having β1-4 linkages to glucose subunits having β1-3 linkages is between 2:1 to 4:1 (e.g., 2:1 to 2.5:1, 2:1 to 3:1, 2:1 to 3.5:1, 2.5:1 to 3:1, 2.5:1 to 3.5:1, 2.5:1 to 4:1, 3:1 to 3.5:1, 3:1 to 4:1, or 3.5:1 to 4:1).

Aspect A78: The method of any one of aspects A66-A77, or any preceding aspect, wherein (1) 30 wt. % to 45 wt. % (e.g., 30-32, 30-35, 30-37, 30-40, 30-42, 32-35, 32-37, 32-40, 32-42, 32-45, 35-37, 35-40, 35-42, 35-45, 37-40, 37-42, 37-45, 40-42, 40-45, or 42-45 wt. %) of the non-terminal glucose subunits are $31-3 linked, based on total weight of non-terminal saccharide subunits.

Aspect A79: The method of any one of aspects A66-A78, or any preceding aspect, wherein the oligosaccharide composition comprises:
a sum of mannose subunits in an amount of 33 wt. % to 70 wt. % (e.g., 33-40, 33-50, 33-60, 40-50, 40-60, 40-70, 50-60, 50-70, or wt. %), based on total weight of saccharide subunits; and a sum of glucose subunits in an amount of 18 wt. % to 45 wt. % (e.g., 18-25, 18-30, 18-35, 18-40, 25-30, 25-35, 25-40, 25-45, 30-35, 30-40, 30-45, 35-40, 35-45, or 40-45 wt. %), based on total weight of saccharide subunits; and
non-terminal glucose subunits and non-terminal mannose subunits, wherein at least 70 wt. % (e.g., at least any of the following: 75, 80, 85, 90, 95, 97, or 99 wt. %; optionally essentially all; optionally 100 wt. %; optionally less than any of the following: 100, 99, 97, 95, 90, 85, 80, or 75 wt. %; optionally where any such values can be combined in any manner to form a range, such as 85-99 wt. % or 90 wt. % to essentially all) of the non-terminal glucose subunits and non-terminal mannose subunits have at least one 4-linkage.

Aspect A80: The method of aspect A79, or any preceding aspect, wherein at least 70 wt. % (e.g., at least any of the following: 75, 80, 85, 90, 95, 97, or 99 wt. %; optionally essentially all; optionally 100 wt. %; optionally less than any of the following: 100, 99, 97, 95, 90, 85, 80, or 75 wt. %; optionally where any such values can be combined in any manner to form a range, such as 85-99 wt. % or 90 wt. % to essentially all) of the non-terminal glucose subunits and non-terminal mannose subunits have β1-4 linkages.

Aspect A81: The method of any one of aspects A66-A80, or any preceding aspect, wherein the oligosaccharide composition comprises:
a sum of xylose subunits in an amount of 33 wt. % to 70 wt. % (e.g., 33-40, 33-50, 33-60, 40-50, 40-60, 40-70, 50-60, 50-70, or wt. %), based on total weight of saccharide subunits; and a sum of arabinose subunits in an amount of 18 wt. % to 45 wt. % (e.g., 18-25, 18-30, 18-35, 18-40, 25-30, 25-35, 25-40, 25-45, 30-35, 30-40, 30-45, 35-40, 35-45, or 40-45 wt. %), based on total weight of saccharide subunits;
non-terminal xylose subunits, wherein at least 70 wt. % (e.g., at least any of the following: 75, 80, 85, 90, 95, 97, or 99 wt. %; optionally essentially all; optionally 100 wt. %; optionally less than any of the following: 100, 99, 97, 95, 90, 85, 80, or 75 wt. %; optionally where any such values can be combined in any manner to form a range, such as 85-99 wt. % or 90 wt. % to essentially all) the non-terminal xylose subunits have at least one 4-linkage; and
arabinose subunits, wherein at least 70 wt. % (e.g., at least any of the following: 75, 80, 85, 90, 95, 97, or 99 wt. %; optionally essentially all; optionally 100 wt. %; optionally less than any of the following: 100, 99, 97, 95, 90, 85, 80, or 75 wt. %; optionally where any such values can be combined in any manner to form a range, such as 85-99 wt. % or 90 wt. % to essentially all) of the arabinose subunits are terminal arabinose subunits.

Aspect A82: The method of aspect A81, or any preceding aspect, wherein at least 70 wt. % (e.g., at least any of the following: 75, 80, 85, 90, 95, 97, or 99 wt. %; optionally essentially all; optionally 100 wt. %; optionally less than any of the following: 100, 99, 97, 95, 90, 85, 80, or 75 wt. %; optionally where any such values can be combined in any manner to form a range, such as 85-99 wt. % or 90 wt. % to essentially all) the non-terminal xylose subunits have β1-4 linkages.

Aspect A83: The method of any one of aspects A66-A82, or any preceding aspect, wherein the oligosaccharide composition is derived from a material comprising konjac, wheat, corn, rye, oat, barley, pea, carrot, olive, or any combination thereof.

Aspect A84: The method of any one of aspects A66-A83, or any preceding aspect, wherein the oligosaccharide composition is derived from a material comprising glucomannan, arabinoxylan, cereal beta glucan, arabinan, arabinogalactan, pectin, or any combination thereof.

Aspect A85: The method of any one of aspects A66-A84, or any preceding aspect, wherein the oligosaccharide composition comprises CLX102, CLX114, CLX115, CLX115-AL, CLX115-FC, CLX122-DS, CLX122-DSF, CLX122, CLX127, CLX131, or any combination thereof.

Aspect A86: The method of any one of aspects A66-A85, or any preceding aspect, wherein the method increases abundance of the indole derivative-producing microbiota.

Aspect A87: The method of any one of aspects A66-A86, or any preceding aspect, wherein the method modulates the microbiota to produce the at least one indole derivative.

Aspect A88: The method of any one of aspects A66-A87, or any preceding aspect, wherein the at least one indole derivative comprises indole-3-aldehyde, indole-3-lactate, indole-3-propionate, or any combination thereof.

Aspect A89: The method of any one of aspects A66-A88, or any preceding aspect, wherein the method causes the microbiota to produce an increased amount of the at least one indole derivative compared to an otherwise identical method that excludes contacting the microbiota with the formulation comprising the oligosaccharide composition.

Aspect A90: The method of any one of aspects A66-A89, or any preceding aspect, wherein the microbiota comprise:
Bifidobacterium, optionally selected from Bifidobacterium longum, Bifidobacterium pseudocatenulatum, Bifidobacterium lactis, Bifidobacterium infantus, Bifidobacterium adolescentis, or Bifidobacterium animalis, or any combination thereof;
Lactobacilli, optionally selected from Lactobacillus lactis, Lactobacillus rhamnosus, or Lactobacillus plantarum, or any combination thereof;
Clostridium sporongenes or Peptosreptococcus, or a combination thereof; or any combination thereof.

Aspect A91: The method of any one of aspects A66-A90, or any preceding aspect, wherein the method modulates the microbiota to metabolize bile acids and/or bile salts.

Aspect A92: The method of any one of aspects A66-A91, or any preceding aspect, wherein the bile acids and/or bile salts comprise cholate, deoxycholate, lithocholate, chenodeoxycholate, ursodeoxycholate, 7-oxolithocholate, 7-oxo-deoxycholate, or any combination thereof.

Aspect A93: The method of any one of aspects A66-A92, or any preceding aspect, wherein the bile acid or bile salt metabolizing microbiota comprise:
Clostridium cluster XIVa, optionally selected from Clostridium scindens, Clostridium hiranonis, or Clostridium hylemonae, or any combination thereof;
Clostridium cluster XI, optionally selected from Clostridium sordelli; or
Eubacterium spp.; or
any combination thereof.

Aspect A94: The method of any one of aspects A91-A93, or any preceding aspect, wherein the method modulates the microbiota to metabolize bile salts into bile acids, wherein the bile acids comprise primary bile acids, secondary bile acids, or a combination thereof.

Aspect A95: The method of aspect A94, or any preceding aspect, wherein:
the primary bile acids comprise cholate, chenodeoxycholate, or a combination thereof; and
the secondary bile acids comprise deoxycholate, lithocholate, ursodeoxycholate, 7-oxolithocholate, 7-oxo-deoxycholate, or any combination thereof.

Aspect A96: The method of any one of aspects A66-A95, or any preceding aspect, wherein the method increases abundance of the microbiota.

Aspect A97: The method of any one of aspects A66-A96, or any preceding aspect, wherein the microbiota are located in a gastrointestinal tract of a subject, and the contacting step comprises administering the formulation to the subject.

Aspect A98: The method of aspect A97, or any preceding aspect, wherein the subject is in need of the formulation to prevent or treat a disease, condition, disorder, and/or indication, and the contacting step comprises administering to the subject a therapeutically effective amount of the formulation.

Aspect A99: The method of aspect A97 or A98, or any preceding aspect, wherein the method lowers inflammation in the gastrointestinal tract of the subject.

Aspect A100: The method of any one of aspects A97-A99, or any preceding aspect, wherein the method lowers at least one of inflammatory gastrointestinal markers TNF-α, IL1-β, or IL-6, or any combination thereof, in the subject.

Aspect A101: The method of any one of aspects A97-A100, or any preceding aspect, wherein the method increases at least one of anti-inflammatory gastrointestinal markers IL-10, TGF-beta, or IL-4, or any combination thereof, in the subject.

Aspect A102: The method of any one of aspects A97-A101, or any preceding aspect, wherein the method increases the production of GLP-1 in the subject.

Aspect A103: The method of any one of aspects A97-A102, or any preceding aspect, wherein the method decreases intestinal barrier permeability in the gastrointestinal tract of the subject.

Aspect A104: The method of any one of aspects A97-A103, or any preceding aspect, wherein the method increases the expression of Muc2, occluding, claudin-4, or ZO-1 genes, or any combination thereof, in the gastrointestinal tract of the subject.

Aspect A105: The method of any one of aspects A97-A104, or any preceding aspect, wherein the method lowers A1c levels in the subject.

Aspect A106: The method of any one of aspects A97-A105, or any preceding aspect, wherein the disease, condition, disorder, and/or indication is type 2 diabetes mellitus, atherosclerosis, IBD, Crohn's, NAFLD, Constipation, Celiac Disease, Diarrhea, IBS, or any combination thereof.

Aspect A107: The method of any one of aspects A97-A106, or any preceding aspect, wherein the method increases production of the at least one indole derivative selected from indole-3-aldehyde, indole-3-lactate, or indole-3-propionate, or any combination thereof, in the gastrointestinal tract of the subject.

Aspect A108: The method of any one of aspects A97-A107, or any preceding aspect, wherein the method stimulates at least one of receptors PXR or AhR, or a combination thereof, in the subject.

Aspect A109: The method of any one of aspects A97-A106, or any preceding aspect, wherein the method modulates the microbiota to metabolize bile salts into bile acids in the gastrointestinal tract of the subject.

Aspect A110: The method of any one of aspects A97-A106 or A109, or any preceding aspect, wherein the method stimulates receptor FXR or TGR5, or a combination thereof, in the subject.

Aspect A111: The method of any one of aspects A97-A106, A109, or A110, or any preceding aspect, wherein the method induces 5-HT biosynthesis in the subject.

Aspect A112: An oligosaccharide composition, comprising:
a sum of galactose and arabinose subunits in an amount of at least 33 wt. % (e.g., at least any of the following: 35, 40, 45, 50, 55, 60, 65, 70, 75, 80, 85, or 90 wt. %; optionally less than any of the following: 95, 90, 85, 80, 75, 70, 65, 60, 55, 50, 45, 40, or 35 wt. %; optionally wherein any of such values can be combined in any manner to form a range, such as 33-80 wt. %), based on total weight of saccharide subunits.

Aspect A113: The oligosaccharide composition of aspect A112, or any preceding aspect, comprising at least 50 wt. % (e.g., at least any of the following: 50, 55, 60, 65, 70, 75, 80, 85, 90, 95, or 99 wt. %; optionally 100 wt. %; optionally less than any of the following: 100, 99, 95, 90, 85, 80, 75, 70, 65, 60, or 55 wt. %; optionally wherein any of such values can be combined in any manner to form a range, such as 55-90 wt. %) oligosaccharides on a dry basis having a degree of polymerization of between 3 and 50 monosaccharide subunits (e.g., 3-5, 3-8, 3-10, 3-12, 3-15, 3-18, 3-20, 3-25, 3-30, 3-35, 3-40, 3-45, 5-8, 5-10, 5-12, 5-15, 5-18, 5-20, 5-25, 5-30, 5-35, 5-40, 5-45, 5-50, 10-15, 10-20, 10-25, 10-30, 10-35, 10-40, 10-45, 10-50, 15-20, 15-25, 15-30, 15-35, 15-40, 15-45, 15-50, 20-25, 20-30, 20-35, 20-40, 20-45, 20-50, 25-30, 25-35, 25-40, 25-45, 25-50, 30-35, 30-40, 30-45, 30-50, 35-40, 35-45, 35-50, 40-45, 40-50, or 45-50).

Aspect A114: The oligosaccharide composition of aspect A112 or A113, or any preceding aspect, comprising a dynamic viscosity at 25° C. of between 0.8 and 2.5 (e.g., 0.8-1, 0.8-1.2, 0.8-1.5, 0.8-1.7, 0.8-2, 0.8-2.2, 1-1.2, 1-1.5, 1-1.7, 1-2, 1-2.2, 1-2.5, 1.2-1.5, 1.2-1.7, 1.2-2, 1.2-2.2, 1.2-2.5, 1.5-1.7, 1.5-2, 1.5-2.2, 1.5-2.5, 1.7-2, 1.7-2.2, 1.7-2.5, 2-2.2, 2-2.5, or 2.2-2.5) mPa*s at a concentration of 100 mg of the oligosaccharide composition on a dry basis in 1 mL of water (i.e., per mL).

Aspect A115: The oligosaccharide composition of any one of aspects A112-A114, or any preceding aspect, comprising at least 5 wt. % (e.g., at least any of the following: 5, 7, 8, 10, 12, 15, 20, 25, or 30 wt. %; optionally less than any of the following: 35, 30, 25, 20, 15, 12, 10, 8, or 7 wt. %; optionally wherein any of such values can be combined in any manner to form a range, such as 5-25 wt. % or 8-15 wt. %) arabinose subunits and at least 5 wt. % (e.g., at least any of the following: 5, 7, 8, 10, 12, 15, 20, 25, or 30 wt. %; optionally less than any of the following: 35, 30, 25, 20, 15, 12, 10, 8, or 7 wt. %; optionally wherein any of such values can be combined in any manner to form a range, such as 5-30 wt. % or 8-25 wt. %) galactose subunits, based on total weight of saccharide subunits.

Aspect A116: The oligosaccharide composition of any one of aspects A112-A115, or any preceding aspect, comprising a sum of galactose subunits and arabinose subunits in an amount of at least 50 wt. % (e.g., at least any of the following: 50, 55, 60, 65, 70, 75, 80, 85, 90, 95, or 99 wt. %; optionally 100 wt. %; optionally less than any of the following: 100, 99, 95, 90, 85, 80, 75, 70, 65, 60, or 55 wt. %; optionally wherein any of such values can be combined in any manner to form a range, such as 55-90 wt. %), based on total weight of saccharide subunits.

Aspect A117: The oligosaccharide composition of any one of aspects A112-A116, or any preceding aspect, comprising non-terminal arabinose subunits, wherein at least 70% wt. % (e.g., at least any of the following: 75, 80, 85, 90, 95, 97, or 99 wt. %; optionally essentially all; optionally 100 wt. %; optionally less than any of the following: 100, 99, 97, 95, 90, 85, 80, or 75 wt. %; optionally where any such values can be combined in any manner to form a range, such as 85-99 wt. % or 90 wt. % to essentially all) of the non-terminal arabinose subunits have at least one 5-linkage.

Aspect A118: The oligosaccharide composition of any one of aspects A112-A117, or any preceding aspect, comprising non-terminal galactose subunits, wherein at least 70% wt. % (e.g., at least any of the following: 75, 80, 85, 90, 95, 97, or 99 wt. %; optionally essentially all; optionally 100 wt. %; optionally less than any of the following: 100, 99, 97, 95, 90, 85, 80, or 75 wt. %; optionally where any such values can be combined in any manner to form a range, such as 85-99 wt. % or 90 wt. % to essentially all) of the non-terminal galactose subunits have at least one 4-linkage.

Aspect A119: The oligosaccharide composition of any one of aspects A112-A117, or any preceding aspect, comprising non-terminal galactose subunits, wherein at least 33% wt. % (e.g., at least any of the following: 35, 40, 45, 50, 55, 60, 65, 70, 75, 80, 85, or 90 wt. %; optionally less than any of the following: 95, 90, 85, 80, 75, 70, 65, 60, 55, 50, 45, 40, or 35 wt. %; optionally wherein any of such values can be combined in any manner to form a range, such as 33-55 wt. %) of the non-terminal galactose subunits have a 3,6-linkage.

Aspect A120: The oligosaccharide composition of any one of aspects A112-A119, or any preceding aspect, comprising a sum of rhamnose subunits and galacturonic acid subunits in an amount of 3 wt. % to 30 wt. % (e.g., 3-5, 3-10, 3-15, 3-20, 3-25, 5-10, 5-15, 5-20, 5-25, 5-30, 10-15, 10-20, 10-25, 10-30, 15-20, 15-25, 15-30, 20-25, 20-30, or 25-30 wt. %), based on total weight of saccharide subunits.

Aspect A121: The oligosaccharide composition of any one of aspects A112-A118 or A120, or any preceding aspect, comprising at least one of (e.g., at least two of, at least three of, at least four of, at least five of, at least six of, at least seven of, or at least eight of): (a) at least two (e.g., at least 3, 4, 5, 6, 7, 8, or 9; optionally less than 10, 9, 8, 7, 6, 5, 4, or 3; optionally wherein any of such values can be combined in any manner to form a range, such as 2-9) different isomers of 2Hex1Pent, (b) at least two (e.g., at least 3, 4, 5, 6, 7, 8, or 9; optionally less than 10, 9, 8, 7, 6, 5, 4, or 3; optionally wherein any of such values can be combined in any manner to form a range, such as 2-9) different isomers of 3Hex, (c) at least one (e.g., at least 2, 3, 4, 5, 6, 7, 8, or 9; optionally less than 10, 9, 8, 7, 6, 5, 4, 3, or 2; optionally wherein any of such values can be combined in any manner to form a range, such as 2-9) [different] isomer[s] of 3Hex1Pent, (d) at least three (e.g., at least 3, 4, 5, 6, 7, 8, or 9; optionally less than 10, 9, 8, 7, 6, 5, or 4; optionally wherein any of such values can be combined in any manner to form a range, such as 3-7) different isomers of 4Hex, (e) at least two (e.g., at least 3, 4, 5, 6, 7, 8, or 9; optionally less than 10, 9, 8, 7, 6, 5, 4, or 3; optionally wherein any of such values can be combined in any manner to form a range, such as 2-9) different isomers of 4Hex1Pent, (f) at least one (e.g., at least 2, 3, 4, 5, 6, 7, 8, or 9; optionally less than 10, 9, 8, 7, 6, 5, 4, 3, or 2; optionally wherein any of such values can be combined in any manner to form a range, such as 2-9) [different]isomer[s] of 5Hex, (g) at least one (e.g., at least 2, 3, 4, 5, 6, 7, 8, or 9; optionally less than 10, 9, 8, 7, 6, 5, 4, 3, or 2; optionally wherein any of such values can be combined in any manner to form a range, such as 2-9) [different] isomer[s] of 5Hex1Pent, (h) at least one (e.g., at least 2, 3, 4, 5, 6, 7, 8, or 9; optionally less than 10, 9, 8, 7, 6, 5, 4, 3, or 2; optionally wherein any of such values can be combined in any manner to form a range, such as 2-9) [different] isomer[s] of 6Hex, or (i) any combination thereof.

Aspect A122: The oligosaccharide composition of any one of aspects A112-A118, A120, or A121, or any preceding aspect, comprising at least three of: (a) at least two different isomers of 2Hex1Pent, (b) at least two different isomers of 3Hex, (c) at least one isomer of 3Hex1Pent, (d) at least three different isomers of 4Hex, (e) at least two different isomers of 4Hex1Pent, (f) at least one isomer of 5Hex, (g) at least one isomer of 5Hex1Pent, (h) at least one isomer of 6Hex, (i) or any combination thereof.

Aspect A123: The oligosaccharide composition of any one of aspects A112-A118 or A120-A122, or any preceding aspect, comprising at least one of (e.g., at least two of, at least three of, or at least four of): (a) at least six (e.g., at least 6, 7, 8, 9, 10, 11, 12, 13, 14, 15; optionally less than 16, 15, 14, 13, 12, 11, 10, 9, 8, or 7; optionally wherein any of such values can be combined in any manner to form a range, such as 7-15) different isomers of 3Pent, (b) at least six (e.g., at least 6, 7, 8, 9, 10, 11, 12, 13, 14, 15; optionally less than 16, 15, 14, 13, 12, 11, 10, 9, 8, or 7; optionally wherein any of such values can be combined in any manner to form a range, such as 7-15) different isomers of 4Pent, (c) at least three (e.g., at least 3, 4, 5, 6, 7, 8, or 9; optionally less than 10, 9, 8, 7, 6, 5, or 4; optionally wherein any of such values can be combined in any manner to form a range, such as 3-7) different isomers of 5Pent, (d) at least three (e.g., at least 3, 4, 5, 6, 7, 8, or 9; optionally less than 10, 9, 8, 7, 6, 5, or 4; optionally wherein any of such values can be combined in any manner to form a range, such as 3-7) different isomers of 6Pent, or (e) any combination thereof.

Aspect A124: The oligosaccharide composition of any one of aspects A112-A118 or A120-A123, or any preceding aspect, comprising at least three of: (a) at least six different isomers of 3Pent, (b) at least six different isomers of 4Pent, (c) at least three different isomers of 5Pent, (d) at least three different isomers of 6Pent, or (e) any combination thereof.

Aspect A125: The oligosaccharide composition of any one of aspects A112-A118 or A120-A122, or any preceding aspect, comprising at least one of (e.g., at least two of, at least three of, or at least four of): (a) at least one (e.g., at least 2, 3, 4, 5, 6, 7, 8, or 9; optionally less than 10, 9, 8, 7, 6, 5, 4, 3, or 2; optionally wherein any of such values can be combined in any manner to form a range, such as 2-9) [different] isomer[s] of 2Hex2Pent, (b) at least two (e.g., at least 3, 4, 5, 6, 7, 8, or 9; optionally less than 10, 9, 8, 7, 6, 5, 4, or 3; optionally wherein any of such values can be combined in any manner to form a range, such as 2-9) different isomers of 3Hex2Pent, (c) at least one (e.g., at least 2, 3, 4, 5, 6, 7, 8, or 9; optionally less than 10, 9, 8, 7, 6, 5, 4, 3, or 2; optionally wherein any of such values can be combined in any manner to form a range, such as 2-9) [different] isomer[s] of 4Hex2Pent, (d) at least one (e.g., at least 2, 3, 4, 5, 6, 7, 8, or 9; optionally less than 10, 9, 8, 7, 6, 5, 4, 3, or 2; optionally wherein any of such values can be combined in any manner to form a range, such as 2-9) [different] isomer[s] of 7Hex1Pent, or (e) any combination thereof.

Aspect A126: The oligosaccharide composition of any one of aspects A112-A118, A120-A122, or A125, or any preceding aspect, comprising at least two of: (a) at least one isomer of 2Hex2Pent, (b) at least two isomers of 3Hex2Pent, (c) at least one isomer of 4Hex2Pent, (d) at least one isomer of 7Hex1Pent, or (e) any combination thereof.

Aspect A127: The oligosaccharide composition of any one of aspects A112-A117, A119, or A120, or any preceding aspect, comprising at least one of (e.g., at least two of, at least three of, at least four of, at least five of, at least six of, at least seven of, at least eight of, at least nine of, or at least ten of): (a) at least two (e.g., at least 3, 4, 5, 6, 7, 8, or 9; optionally less than 10, 9, 8, 7, 6, 5, 4, or 3; optionally wherein any of such values can be combined in any manner to form a range, such as 2-9) different isomers of 1Hex2Pent, (b) at least three (e.g., at least 3, 4, 5, 6, 7, 8, or 9; optionally less than 10, 9, 8, 7, 6, 5, or 4; optionally wherein any of such values can be combined in any manner to form a range, such as 3-7) different isomers of 2Hex1Pent, (c) at least three (e.g., at least 3, 4, 5, 6, 7, 8, or 9; optionally less than 10, 9, 8, 7, 6, 5, or 4; optionally wherein any of such values can be combined in any manner to form a range, such as 3-7) different isomers of 3Hex, (d) at least two (e.g., at least 3, 4, 5, 6, 7, 8, or 9; optionally less than 10, 9, 8, 7, 6, 5, 4, or 3; optionally wherein any of such values can be combined in any manner to form a range, such as 2-9) different isomers of 3Hex1Pent, (e) at least two (e.g., at least 3, 4, 5, 6, 7, 8, or 9; optionally less than 10, 9, 8, 7, 6, 5, 4, or 3; optionally wherein any of such values can be combined in any manner to form a range, such as 2-9) different isomers of 4Hex, (f) at least two (e.g., at least 3, 4, 5, 6, 7, 8, or 9; optionally less than 10, 9, 8, 7, 6, 5, 4, or 3; optionally wherein any of such values can be combined in any manner to form a range, such as 2-9) different isomers of 4Hex1Pent, (g) at least two (e.g., at least 3, 4, 5, 6, 7, 8, or 9; optionally less than 10, 9, 8, 7, 6, 5, 4, or 3; optionally wherein any of such values can be combined in any manner to form a range, such as 2-9) different isomers of 5Hex, (h) at least one (e.g., at least 2, 3, 4, 5, 6, 7, 8, or 9; optionally less than 10, 9, 8, 7, 6, 5, 4, 3, or 2; optionally wherein any of such values can be combined in any manner to form a range, such as 2-9) [different] isomer[s] of 5Hex1Pent, (i) at least one (e.g., at least 2, 3, 4, 5, 6, 7, 8, or 9; optionally less than 10, 9, 8, 7, 6, 5, 4, 3, or 2; optionally wherein any of such values can be combined in any manner to form a range, such as 2-9) [different] isomer[s] of 6Hex, (j) at least one (e.g., at least 2, 3, 4, 5, 6, 7, 8, or 9; optionally less than 10, 9, 8, 7, 6, 5, 4, 3, or 2; optionally wherein any of such values can be combined in any manner to form a range, such as 2-9) [different] isomer[s] of 7Hex, (k) any combination thereof.

Aspect A128: The oligosaccharide composition of any one of aspects A112-A117, A119, A120, or A127, or any preceding aspect, comprising at least three of: (a) at least two different isomers of 1Hex2Pent, (b) at least three different isomers of 2Hex1Pent, (c) at least three different isomers of 3Hex, (d) at least two different isomers of 3Hex1Pent, (e) at least two different isomers of 4Hex, (f) at least two different isomers of 4Hex1Pent, (g) at least two different isomers of 5Hex, (h) at least one isomer of 5Hex1Pent, (i) at least one isomer of 6Hex, (j) at least one isomer of 7Hex, or (k) any combination thereof.

Aspect A129: The oligosaccharide composition of any one of aspects A112-A117, A119, A120, A127, or A128, or any preceding aspect, comprising at least one of (e.g., at least two of, at least three of, at least four of, or at least five of): (a) at least two (e.g., at least 3, 4, 5, 6, 7, 8, or 9; optionally less than 10, 9, 8, 7, 6, 5, 4, or 3; optionally wherein any of such values can be combined in any manner to form a range, such as 2-9) different isomers of 1Hex3Pent, (b) at least two (e.g., at least 3, 4, 5, 6, 7, 8, or 9; optionally less than 10, 9, 8, 7, 6, 5, 4, or 3; optionally wherein any of such values can be combined in any manner to form a range, such as 2-9) different isomers of 2Hex2Pent, (c) at least five (e.g., at least 5, 6, 7, 8, 9, 10, 11, 12, 13, 14, 15; optionally less than 16, 15, 14, 13, 12, 11, 10, 9, 8, 7, or 6; optionally wherein any of such values can be combined in any manner to form a range, such as 5-12) different isomers of 3Pent, (d) at least three (e.g., at least 3, 4, 5, 6, 7, 8, or 9; optionally less than 10, 9, 8, 7, 6, 5, or 4; optionally wherein any of such values can be combined in any manner to form a range, such as 3-7) different isomers of 4Pent, (e) at least one (e.g., at least 2, 3, 4, 5, 6, 7, 8, or 9; optionally less than 10, 9, 8, 7, 6, 5, 4, 3, or 2; optionally wherein any of such values can be combined in any manner to form a range, such as 2-9) [different] isomer[s] of 6Pent, or (f) any combination thereof.

Aspect A130: The oligosaccharide composition of any one of aspects A112-A117, A119, A120, or A127-A129, or any preceding aspect, comprising at least two of: (a) at least two different isomers of 1Hex3Pent, (b) at least two different isomers of 2Hex2Pent, (c) at least five different isomers of 3Pent, (d) at least three different isomers of 4Pent, (e) at least one isomer of 6Pent, or (f) any combination thereof.

Aspect A131: The oligosaccharide composition of any one of aspects A112-A117, A119, A120, A127, or A128, or any preceding aspect, comprising at least one of (e.g., at least two of, at least three of, or at least four of): (a) at least five (e.g., at least 5, 6, 7, 8, 9, 10, 11, 12, 13, 14, 15; optionally less than 16, 15, 14, 13, 12, 11, 10, 9, 8, 7, or 6; optionally wherein any of such values can be combined in any manner to form a range, such as 5-12) different isomers of 3Hex, (b) at least six (e.g., at least 6, 7, 8, 9, 10, 11, 12, 13, 14, 15; optionally less than 16, 15, 14, 13, 12, 11, 10, 9, 8, or 7; optionally wherein any of such values can be combined in any manner to form a range, such as 7-15) different isomers of 4Hex, (c) at least three (e.g., at least 3, 4, 5, 6, 7, 8, or 9; optionally less than 10, 9, 8, 7, 6, 5, or 4; optionally wherein any of such values can be combined in any manner to form a range, such as 3-7) different isomers of 5Hex, (d) at least two (e.g., at least 3, 4, 5, 6, 7, 8, or 9; optionally less than 10, 9, 8, 7, 6, 5, 4, or 3; optionally wherein any of such values can be combined in any manner to form a range, such as 2-9) different isomers of 6Hex, or (e) any combination thereof.

Aspect A132: The oligosaccharide composition of any one of aspects A112-A117, A119, A120, A127, A128, or A131, or any preceding aspect, comprising at least two of: (a) at least five different isomers of 3Hex, (b) at least six different isomers of 4Hex, (c) at least three different isomers of 5Hex, (d) at least two different isomers of 6Hex, or (e) any combination thereof.

Aspect A133: The oligosaccharide composition of any one of aspects A112-A117, A119, A120, A127, or A128, or any preceding aspect, comprising at least one of (e.g., at least two of, at least three of, or at least four of): (a) at least four (e.g., at least 4, 5, 6, 7, 8, 9, 10, 11, 12, 13, 14, 15; optionally less than 16, 15, 14, 13, 12, 11, 10, 9, 8, 7, 6, or 5; optionally wherein any of such values can be combined in any manner to form a range, such as 8-16) different isomers of 2Hex1Pent, (b) at least four (e.g., at least 4, 5, 6, 7, 8, 9, 10, 11, 12, 13, 14, 15; optionally less than 16, 15, 14, 13, 12, 11, 10, 9, 8, 7, 6, or 5; optionally wherein any of such values can be combined in any manner to form a range, such as 8-16) different isomers of 2Hex2Pent, (c) at least five (e.g., at least 5, 6, 7, 8, 9, 10, 11, 12, 13, 14, 15; optionally less than 16, 15, 14, 13, 12, 11, 10, 9, 8, 7, or 6; optionally wherein any of such values can be combined in any manner to form a range, such as 5-12) different isomers of 3Hex1Pent, (d) at least two (e.g., at least 3, 4, 5, 6, 7, 8, or 9; optionally less than 10, 9, 8, 7, 6, 5, 4, or 3; optionally wherein any of such values can be combined in any manner to form a range, such as 2-9) different isomers of 3Hex2Pent, or (e) any combination thereof.

Aspect A134: The oligosaccharide composition of any one of aspects A112-A117, A119, A120, A127, A128, or A133, or any preceding aspect, comprising at least two of: (a) at least four different isomers of 2Hex1Pent, (b) at least four different isomers of 2Hex2Pent, (c) at least five different isomers of 3Hex1Pent, (d) at least two different isomers of 3Hex2Pent, or (e) any combination thereof.

Aspect A135: The oligosaccharide composition of any one of aspects A112-A134, or any preceding aspect, wherein the oligosaccharide composition is within 30% (e.g., within 25%, within 20%, within 15%, within 10%, within 5%, within 3%, or within 1%) of at least 70% (e.g., at least any of the following: 75, 80, 85, 90, 95, 97, or 99%; optionally 100%; optionally less than any of the following: 100, 99, 97, 95, 90, 85, 80, or 75%; optionally where any such values can be combined in any manner to form a range, such as 85-99%) of the entries set forth in one of Tables 22, 24, 25, U3, 27, 29, 30, 34, or 35 on a numbers basis (for example, each value in a Table is compared to the values observed using the same analysis method on the oligosaccharide composition at issue; a determination is then made if each value for the oligosaccharide composition at issue is within 30% (or other % in this paragraph) of each corresponding value in the Table; then the number of entries that are within 30% (or other % in this paragraph) is divided by the total number of entries in the Table to evaluate if at least 70% (or other % in this paragraph) of the values in the Table are within 30% (or other % in this paragraph); this same calculation method can be used elsewhere herein where this same language is used).

Aspect A136: The oligosaccharide composition of any one of aspects A112-A135, or any preceding aspect, wherein the oligosaccharide composition is CLX116, CLX117, CLX119, CLX122, CLX122-DS, CLX122-DSF, CLX124, CLX126, CLX127, CLX131, CLX132, CLX133, or any combination thereof.

Aspect A137: The oligosaccharide composition of any one of aspects A112-A136, or any preceding aspect, wherein the oligosaccharide composition is derived from a material comprising Orange, Astragalus, Tomato, Pea, coffee, Soy, Carrot, Olive, Beet, Baobob, or any combination thereof.

Aspect A138: The oligosaccharide composition of any one of aspects A112-A137, or any preceding aspect, wherein the oligosaccharide composition is derived from a material comprising pectin, tragacanth, arabinan, arabinogalactan, galactomannan, mannan, rhamnogalacturonan I, rhamnogalacturonan II, xylan, or any combination thereof.

Aspect A139: An oligosaccharide composition, comprising:
a sum of 3-linked and 4-linked glucose subunits in an amount of between 5-75 wt. % (e.g., 5-7, 5-10, 5-20, 5-30, 5-40, 5-50, 5-60, 5-70, 7-10, 7-20, 7-30, 7-40, 7-50, 7-60, 7-70, 7-75, 10-20, 10-30, 10-40, 10-50, 10-60, 10-70, 10-75, 20-30, 20-40, 20-50, 20-60, 20-70, 20-75, 30-40, 30-50, 30-60, 30-70, 30-75, 40-50, 40-60, 40-70, 40-75, 50-60, 50-70, 50-75, 60-70, 60-75, or 70-75 wt. %), based on total weight of saccharide subunits.

Aspect A140: The oligosaccharide composition of aspect A139, or any preceding aspect, comprising at least 50 wt. % (e.g., at least any of the following: 50, 55, 60, 65, 70, 75, 80, 85, 90, 95, or 99 wt. %; optionally 100 wt. %; optionally less than any of the following: 100, 99, 95, 90, 85, 80, 75, 70, 65, 60, or 55 wt. %; optionally wherein any of such values can be combined in any manner to form a range, such as 55-90 wt. %) oligosaccharides on a dry basis having a degree of polymerization of between 3 and 50 (e.g., 3-5, 3-8, 3-10, 3-12, 3-15, 3-18, 3-20, 3-25, 3-30, 3-35, 3-40, 3-45, 5-8, 5-10, 5-12, 5-15, 5-18, 5-20, 5-25, 5-30, 5-35, 5-40, 5-45, 5-50, 10-15, 10-20, 10-25, 10-30, 10-35, 10-40, 10-45, 10-50, 15-20, 15-25, 15-30, 15-35, 15-40, 15-45, 15-50, 20-25, 20-30, 20-35, 20-40, 20-45, 20-50, 25-30, 25-35, 25-40, 25-45, 25-50, 30-35, 30-40, 30-45, 30-50, 35-40, 35-45, 35-50, 40-45, 40-50, or 45-50) monosaccharide subunits.

Aspect A141: The oligosaccharide composition of aspect A139 or A140, or any preceding aspect, comprising a dynamic viscosity at 25° C. of between 0.8 and 2.5 (e.g., 0.8-1, 0.8-1.2, 0.8-1.5, 0.8-1.7, 0.8-2, 0.8-2.2, 1-1.2, 1-1.5, 1-1.7, 1-2, 1-2.2, 1-2.5, 1.2-1.5, 1.2-1.7, 1.2-2, 1.2-2.2, 1.2-2.5, 1.5-1.7, 1.5-2, 1.5-2.2, 1.5-2.5, 1.7-2, 1.7-2.2, 1.7-2.5, 2-2.2, 2-2.5, or 2.2-2.5) mPa*s at a concentration of 100 mg of the oligosaccharide composition on a dry basis in 1 mL of water (i.e., per mL).

Aspect A142: The oligosaccharide composition of any one of aspects A139-A141, or any preceding aspect, comprising at least 2 wt. % (e.g., at least any of the following: 2, 3, 4, 5, 7, 10, 12, 15, 20, 25, or 30 wt. %; optionally less than any of the following: 35, 30, 25, 20, 15, 12, 10, 7, 5, 4, 3, or 2 wt. %; optionally wherein any of such values can be combined in any manner to form a range, such as 2-10 wt. %) 3-linked glucose subunits and at least 2 wt. % at least 2 wt. % (e.g., at least any of the following: 2, 3, 4, 5, 7, 10, 12, 15, 20, 25, or 30 wt. %; optionally less than any of the following: 35, 30, 25, 20, 15, 12, 10, 7, 5, 4, 3, or 2 wt. %; optionally wherein any of such values can be combined in any manner to form a range, such as 3-7 wt. %) 4-linked glucose subunits, based on total weight of saccharide subunits.

Aspect A143: The oligosaccharide composition of any one of aspects A139-A142, or any preceding aspect, comprising glucose subunits, wherein at least 70 wt. % (e.g., at least any of the following: 75, 80, 85, 90, 95, 97, or 99 wt. %; optionally essentially all; optionally 100 wt. %; optionally less than any of the following: 100, 99, 97, 95, 90, 85, 80, or 75 wt. %; optionally where any such values can be combined in any manner to form a range, such as 85-99 wt. % or 90 wt. % to essentially all) of the glucose subunits comprise $-linkages.

Aspect A144: The oligosaccharide composition of any one of aspects A139-A143, or any preceding aspect, comprising non-terminal glucose subunits, wherein 10 wt. % to 30 wt. % (e.g., 10-15, 10-20, 10-25, 15-20, 15-25, 15-30, 20-25, 20-30, or 25-30 wt. %) of the non-terminal glucose subunits have at least one 6-linkage.

Aspect A145: The oligosaccharide composition of any one of aspects A139-A144, or any preceding aspect, comprising at least 70 wt. % glucose subunits (e.g., at least any of the following: 75, 80, 85, 90, 95, 97, or 99 wt. %; optionally essentially all; optionally 100 wt. %; optionally less than any of the following: 100, 99, 97, 95, 90, 85, 80, or 75 wt. %; optionally where any such values can be combined in any manner to form a range, such as 85-99 wt. % or 90 wt. % to essentially all), based on total weight of saccharide subunits.

Aspect A146: The oligosaccharide composition of any one of aspects A139-A143, or any preceding aspect, comprising at least 50 wt. % (e.g., at least any of the following: 50, 55, 60, 65, 70, 75, 80, 85, 90, 95, or 99 wt. %; optionally 100 wt. %; optionally less than any of the following: 100, 99, 95, 90, 85, 80, 75, 70, 65, 60, or 55 wt. %; optionally wherein any of such values can be combined in any manner to form a range, such as 55-90 wt. %) mannose subunits, based on total weight of saccharide subunits.

Aspect A147: The oligosaccharide composition of any one of aspects A139-A143 or A146, or any preceding aspect, comprising non-terminal mannose subunits, wherein at least 70 wt. % (e.g., at least any of the following: 75, 80, 85, 90, 95, 97, or 99 wt. %; optionally essentially all; optionally 100 wt. %; optionally less than any of the following: 100, 99, 97, 95, 90, 85, 80, or 75 wt. %; optionally where any such values can be combined in any manner to form a range, such as 85-99 wt. % or 90 wt. % to essentially all) of the non-terminal mannose subunits have at least one 2-linkage.

Aspect A148: The oligosaccharide composition of any one of aspects A139-A143 or A146, or any preceding aspect, comprising glucose subunits, wherein at least 70 wt. % (e.g., at least any of the following: 75, 80, 85, 90, 95, 97, or 99 wt. %; optionally essentially all; optionally 100 wt. %; optionally less than any of the following: 100, 99, 97, 95, 90, 85, 80, or 75 wt. %; optionally where any such values can be combined in any manner to form a range, such as 85-99 wt. % or 90 wt. % to essentially all) of the glucose subunits comprise α-linkages.

Aspect A149: The oligosaccharide composition of any one of aspects A139-A148, or any preceding aspect, comprising at least one of (e.g., at least two of, at least three of, or at least four of): (a) at least four (e.g., at least 4, 5, 6, 7, 8, 9, 10, 11, 12, 13, 14, 15; optionally less than 16, 15, 14, 13, 12, 11, 10, 9, 8, 7, 6, or 5; optionally wherein any of such values can be combined in any manner to form a range, such as 8-16) different isomers of 3Hex, (b) at least three (e.g., at least 3, 4, 5, 6, 7, 8, or 9; optionally less than 10, 9, 8, 7, 6, 5, or 4; optionally wherein any of such values can be combined in any manner to form a range, such as 3-7) different isomers of 4Hex, (c) at least three (e.g., at least 3, 4, 5, 6, 7, 8, or 9; optionally less than 10, 9, 8, 7, 6, 5, or 4; optionally wherein any of such values can be combined in any manner to form a range, such as 3-7) different isomers of 5Hex, or (d) any combination thereof.

Aspect A150: The oligosaccharide composition of any one of aspects A139-A149, or any preceding aspect, comprising at least three of (e.g., at least four of, at least five of, at least six of, at least seven of, or at least eight of): (a) at least four (e.g., at least 4, 5, 6, 7, 8, 9, 10, 11, 12, 13, 14, 15; optionally less than 16, 15, 14, 13, 12, 11, 10, 9, 8, 7, 6, or 5; optionally wherein any of such values can be combined in any manner to form a range, such as 8-16) different isomers of 3Hex, (b) at least three (e.g., at least 3, 4, 5, 6, 7, 8, or 9; optionally less than 10, 9, 8, 7, 6, 5, or 4; optionally wherein any of such values can be combined in any manner to form a range, such as 3-7) different isomers of 4Hex, (c) at least three (e.g., at least 3, 4, 5, 6, 7, 8, or 9; optionally less than 10, 9, 8, 7, 6, 5, or 4; optionally wherein any of such values can be combined in any manner to form a range, such as 3-7) different isomers of 5Hex, or (d) any combination thereof.

Aspect A151: The oligosaccharide composition of any one of aspects A139-A150, or any preceding aspect, comprising at least three of: (a) at least six different isomers of 3Hex, (b) at least six different isomers of 4Hex, (c) at least three different isomers of 5Hex, (d) at least two different isomers of 6Hex, or (e) any combination thereof.

Aspect A152: The oligosaccharide composition of any one of aspects A139-A143, or any preceding aspect, comprising 30-70 wt. % (e.g., 30-35, 30-40, 30-45, 30-50, 30-55, 30-60, 30-65, 35-40, 35-45, 35-50, 35-55, 35-60, 35-65, 35-70, 40-45, 40-50, 40-55, 40-60, 40-65, 40-70, 45-50, 45-55, 45-60, 45-65, 45-70, 50-55, 50-60, 50-65, 50-70, 55-60, 55-65, 55-70, 60-65, 60-70, or 65-70 wt. %) rhamnose subunits, based on total weight of saccharide subunits.

Aspect A153: The oligosaccharide composition of any one of aspects A139-A143 or A152, or any preceding aspect, comprising non-terminal rhamnose subunits, wherein at least 70 wt. % (e.g., at least any of the following: 75, 80, 85, 90, 95, 97, or 99 wt. %; optionally essentially all; optionally 100 wt. %; optionally less than any of the following: 100, 99, 97, 95, 90, 85, 80, or 75 wt. %; optionally where any such values can be combined in any manner to form a range, such as 85-99 wt. % or 90 wt. % to essentially all) of the non-terminal rhamnose subunits have at least one 4-linkage.

Aspect A154: The oligosaccharide composition of any one of aspects A139-A143, A152, or A153, or any preceding aspect, comprising non-terminal galacturonic acid subunits, wherein at least 70 wt. % (e.g., at least any of the following: 75, 80, 85, 90, 95, 97, or 99 wt. %; optionally essentially all; optionally 100 wt. %; optionally less than any of the following: 100, 99, 97, 95, 90, 85, 80, or 75 wt. %; optionally where any such values can be combined in any manner to form a range, such as 85-99 wt. % or 90 wt. % to essentially all) of the non-terminal galacturonic acid subunits have at least one 4-linkage.

Aspect A155: The oligosaccharide composition of any one of aspects A139-A143 or A152, or any preceding aspect, comprising 8-25 wt. % (8-10, 8-12, 8-15, 8-17, 8-20, 8-22, 10-12, 10-15, 10-17, 10-20, 10-22, 10-25, 12-15, 12-17, 12-20, 12-22, 12-25, 15-17, 15-20, 15-22, 15-25, 17-20, 17-22, 17-25, 20-22, 20-25, or 22-25 wt. %) xylose subunits, based on total weight of saccharide subunits.

Aspect A156: The oligosaccharide composition of any one of aspects A139-A143, A152, or A155, or any preceding aspect, comprising non-terminal xylose subunits, wherein at least 60% wt. % (e.g., at least any of the following: 65, 70, 75, 80, 85, 90, 95, or 99 wt. %; optionally 100 wt. %; optionally less than any of the following: 100, 99, 95, 90, 85, 80, 75, 70, or 65 wt. %; optionally wherein any of such values can be combined in any manner to form a range, such as 65-90 wt. %) of the non-terminal xylose subunits have at least one 4-linkage.

Aspect A157: The oligosaccharide composition of any one of aspects A139-A143 or A152-A154, or any preceding aspect, comprising at least one of (e.g., at least two of, at least three of, at least four of, at least five of, or at least six of): (a) at least one (e.g., at least 2, 3, 4, 5, 6, 7, 8, or 9; optionally less than 10, 9, 8, 7, 6, 5, 4, 3, or 2; optionally wherein any of such values can be combined in any manner to form a range, such as 2-9) [different] isomer[s] of 1Hex1HexA1Deoxyhex, (b) at least one (e.g., at least 2, 3, 4, 5, 6, 7, 8, or 9; optionally less than 10, 9, 8, 7, 6, 5, 4, 3, or 2; optionally wherein any of such values can be combined in any manner to form a range, such as 2-9) [different]isomer[s] of 2Hex1HexA, (c) at least three (e.g., at least 3, 4, 5, 6, 7, 8, or 9; optionally less than 10, 9, 8, 7, 6, 5, or 4; optionally wherein any of such values can be combined in any manner to form a range, such as 3-7) different isomers of 2Hex1HexA1Deoxyhex, (d) at least two (e.g., at least 3, 4, 5, 6, 7, 8, or 9; optionally less than 10, 9, 8, 7, 6, 5, 4, or 3; optionally wherein any of such values can be combined in any manner to form a range, such as 2-9) different isomers of 3Hex, (e) at least two (e.g., at least 3, 4, 5, 6, 7, 8, or 9; optionally less than 10, 9, 8, 7, 6, 5, 4, or 3; optionally wherein any of such values can be combined in any manner to form a range, such as 2-9) different isomers of 3Hex1HexA1DeoxyHex, (f) at least three (e.g., at least 3, 4, 5, 6, 7, 8, or 9; optionally less than 10, 9, 8, 7, 6, 5, or 4; optionally wherein any of such values can be combined in any manner to form a range, such as 3-7) different isomers of 4Hex, or (g) any combination thereof.

Aspect A158: The oligosaccharide composition of any one of aspects A139-A143, A152-A154, or A157, or any preceding aspect, comprising at least three of: (a) at least one (e.g., at least 2, 3, 4, 5, 6, 7, 8, or 9; optionally less than 10, 9, 8, 7, 6, 5, 4, 3, or 2; optionally wherein any of such values can be combined in any manner to form a range, such as 2-9) [different] isomer[s] of 1Hex1HexA1Deoxyhex, (b) at least one (e.g., at least 2, 3, 4, 5, 6, 7, 8, or 9; optionally less than 10, 9, 8, 7, 6, 5, 4, 3, or 2; optionally wherein any of such values can be combined in any manner to form a range, such as 2-9) [different] isomer[s] of 2Hex1HexA, (c) at least three (e.g., at least 3, 4, 5, 6, 7, 8, or 9; optionally less than 10, 9, 8, 7, 6, 5, or 4; optionally wherein any of such values can be combined in any manner to form a range, such as 3-7) different isomers of 2Hex1HexA1Deoxyhex, (d) at least two (e.g., at least 3, 4, 5, 6, 7, 8, or 9; optionally less than 10, 9, 8, 7, 6, 5, 4, or 3; optionally wherein any of such values can be combined in any manner to form a range, such as 2-9) different isomers of 3Hex, (e) at least two (e.g., at least 3, 4, 5, 6, 7, 8, or 9; optionally less than 10, 9, 8, 7, 6, 5, 4, or 3; optionally wherein any of such values can be combined in any manner to form a range, such as 2-9) different isomers of 3Hex1HexA1DeoxyHex, (f) at least three (e.g., at least 3, 4, 5, 6, 7, 8, or 9; optionally less than 10, 9, 8, 7, 6, 5, or 4; optionally wherein any of such values can be combined in any manner to form a range, such as 3-7) different isomers of 4Hex, or (g) any combination thereof.

Aspect A159: The oligosaccharide composition of any one of aspects A139-A143, or any preceding aspect, comprising 15-45 wt. % (e.g., 15-25, 15-30, 15-35, 15-40, 25-30, 25-35, 25-40, 25-45, 30-35, 30-40, 30-45, 35-40, 35-45, or 40-45 wt. %) mannose subunits, based on total weight of saccharide subunits.

Aspect A160: The oligosaccharide composition of any one of aspects A139-A143 or A159, or any preceding aspect, comprising non-terminal mannose subunits, wherein at least 60 wt. % (e.g., at least any of the following: 65, 70, 75, 80, 85, 90, 95, or 99 wt. %; optionally 100 wt. %; optionally less than any of the following: 100, 99, 95, 90, 85, 80, 75, 70, or 65 wt. %; optionally wherein any of such values can be combined in any manner to form a range, such as 65-90 wt. %) of the non-terminal mannose subunits have at least one 2-linkage.

Aspect A161: The oligosaccharide composition of any one of aspects [0795]-[0799], [0815], or [0816], or any preceding aspect, comprising non-terminal mannose subunits, wherein at least 20 wt. % (e.g., at least any of the following: 22, 24, 26, 28, 30, 32, 35, 37, 40, 42, 45, or 47 wt. %; optionally less than any of the following: 50, 47, 45, 42, 40, 37, 35, 32, 30, 28, 26, or 24 wt. %; optionally wherein any of such values can be combined in any manner to form a range, such as 30-47 wt. %) of the non-terminal mannose subunits have at least one 4-linkage and at least one 6-linkage.

Aspect A162: The oligosaccharide composition of any one of aspects A139-A143 or A159-A161, or any preceding aspect, comprising at least one of (e.g., at least two of, at least three of, at least four of, at least five of, at least six of, at least seven of, or at least eight of): (a) at least four (e.g., at least 4, 5, 6, 7, 8, 9, 10, 11, 12, 13, 14, 15; optionally less than 16, 15, 14, 13, 12, 11, 10, 9, 8, 7, 6, or 5; optionally wherein any of such values can be combined in any manner to form a range, such as 8-16) different isomers of 2Hex1Pent, (b) at least seven (e.g., at least 7, 8, 9, 10, 11, 12, 13, 14, 15, 16, or 17; optionally less than 18, 16, 15, 14, 13, 12, 11, 10, 9, or 8; optionally wherein any of such values can be combined in any manner to form a range, such as 7-18) different isomers of 3Hex, (c) at least two (e.g., at least 3, 4, 5, 6, 7, 8, or 9; optionally less than 10, 9, 8, 7, 6, 5, 4, or 3; optionally wherein any of such values can be combined in any manner to form a range, such as 2-9) different isomers of 3Hex1HexA, (d) at least four (e.g., at least 4, 5, 6, 7, 8, 9, 10, 11, 12, 13, 14, 15; optionally less than 16, 15, 14, 13, 12, 11, 10, 9, 8, 7, 6, or 5; optionally wherein any of such values can be combined in any manner to form a range, such as 8-16) different isomers of 3Hex1Pent, (e) at least eight (e.g., at least 8, 9, 10, 11, 12, 13, 14, 15, 16, 17, 18, 19, or 20; optionally less than 21, 20, 19, 18, 16, 15, 14, 13, 12, 11, 10, 9, or 8; optionally wherein any of such values can be combined in any manner to form a range, such as 9-14) different isomers of 4Hex, (f) at least one (e.g., at least 2, 3, 4, 5, 6, 7, 8, or 9; optionally less than 10, 9, 8, 7, 6, 5, 4, 3, or 2; optionally wherein any of such values can be combined in any manner to form a range, such as 2-9) [different] isomer[s] of 4Hex1HexA, (g) at least three (e.g., at least 3, 4, 5, 6, 7, 8, or 9; optionally less than 10, 9, 8, 7, 6, 5, or 4; optionally wherein any of such values can be combined in any manner to form a range, such as 3-7) different isomers of 4Hex1Pent, (h) at least eight (e.g., at least 8, 9, 10, 11, 12, 13, 14, 15, 16, 17, 18, 19, or 20; optionally less than 21, 20, 19, 18, 16, 15, 14, 13, 12, 11, 10, 9, or 8; optionally wherein any of such values can be combined in any manner to form a range, such as 9-14) different isomers of 5Hex, or (i) any combination thereof.

Aspect A163: The oligosaccharide composition of any one of A139-A143 or A159-A162, or any preceding aspect, comprising at least four of: (a) at least four different isomers of 2Hex1Pent, (b) at least seven different isomers of 3Hex, (c) at least two different isomers of 3Hex1HexA, (d) at least four different isomers of 3Hex1Pent, (e) at least eight different isomers of 4Hex, (f) at least one isomer of 4Hex1HexA, (g) at least three different isomers of 4Hex1Pent, (h) at least eight different isomers of 5Hex, or (i) any combination thereof.

Aspect A164: The oligosaccharide composition of any one of aspects A139-A143, or any preceding aspect, comprising 35-65 wt. % (e.g., 35-40, 35-45, 35-50, 35-55, 35-60, 35-65, 40-45, 40-50, 40-55, 40-60, 40-65, 45-50, 45-55, 45-60, 45-65, 50-55, 50-60, 50-65, 55-60, 55-65, or 60-65 wt. %) xylose subunits, based on total weight of saccharide subunits.

Aspect A165: The oligosaccharide composition of any one of aspects A139-A143 or A164, or any preceding aspect, comprising 8-25 wt. % (8-10, 8-12, 8-15, 8-17, 8-20, 8-22, 10-12, 10-15, 10-17, 10-20, 10-22, 10-25, 12-15, 12-17, 12-20, 12-22, 12-25, 15-17, 15-20, 15-22, 15-25, 17-20, 17-22, 17-25, 20-22, 20-25, or 22-25 wt. %) arabinose subunits, based on total weight of saccharide subunits.

Aspect A166: The oligosaccharide composition of any one of aspects A139-A143, A164, or A165, or any preceding aspect, comprising non-terminal xylose subunits, wherein at least 70 wt. % (e.g., at least any of the following: 75, 80, 85, 90, 95, 97, or 99 wt. %; optionally essentially all; optionally 100 wt. %; optionally less than any of the following: 100, 99, 97, 95, 90, 85, 80, or 75 wt. %; optionally where any such values can be combined in any manner to form a range, such as 85-99 wt. % or 90 wt. % to essentially all) of the non-terminal xylose subunits have at least one 4-linkage.

Aspect A167: The oligosaccharide composition of any one of aspects A139-A143 or A164-A166, or any preceding aspect, comprising non-terminal arabinose subunits, wherein at least 70 wt. % (e.g., at least any of the following: 75, 80, 85, 90, 95, 97, or 99 wt. %; optionally essentially all; optionally 100 wt. %; optionally less than any of the following: 100, 99, 97, 95, 90, 85, 80, or 75 wt. %; optionally where any such values can be combined in any manner to form a range, such as 85-99 wt. % or 90 wt. % to essentially all) of the non-terminal arabinose subunits have at least one 5-linkage.

Aspect A168: The oligosaccharide composition of any one of aspects A139-A143 or A164-A167, or any preceding aspect, comprising at least four of (e.g., at least five of or at least six of): (a) at least two different isomers of 3Hex, (b) at least two different isomers of 3Pent, (c) at least three different isomers of 4Hex, (d) at least three different isomers of 4Pent, (e) at least three different isomers of 5Hex, (f) at least three different isomers of 5Pent, or (g) any combination thereof.

Aspect A169: The oligosaccharide composition of any one of aspects A139-A143 or A164-A168, or any preceding aspect, comprising at least three of: (a) at least two different isomers of 3Hex, (b) at least two different isomers of 3Pent, (c) at least three different isomers of 4Hex, (d) at least three different isomers of 4Pent, (e) at least three different isomers of 5Hex, (f) at least three different isomers of 5Pent, or (g) any combination thereof.

Aspect A170: The oligosaccharide composition of any one of aspects A139-A169, or any preceding aspect, wherein the oligosaccharide composition is within 30% (e.g., within 25%, within 20%, within 15%, within 10%, within 5%, within 3%, or within 1%) of at least 70% (e.g., at least any of the following: 75, 80, 85, 90, 95, 97, or 99%; optionally 100%; optionally less than any of the following: 100, 99, 97, 95, 90, 85, 80, or 75%; optionally where any such values can be combined in any manner to form a range, such as 85-99%) of the entries set forth in one of Tables 26, 28, or 31 on a numbers basis.

Aspect A171: The oligosaccharide composition of any one of aspects A139-A170, or any preceding aspect, wherein the oligosaccharide composition is CLX 123, CLX 125, CLX 128, CLX 130, or any combination thereof.

Aspect A172: The oligosaccharide composition of any one of aspects A139-A171, or any preceding aspect, wherein the oligosaccharide composition is derived from a material comprising Saccharomyces cerevisiae, Xanthomonas campestris, Sphingomonas, Sugar cane, Kelp, or any combination thereof.

Aspect A173: The oligosaccharide composition of any one of aspects A139-A172, or any preceding aspect, wherein the oligosaccharide composition is derived from a material comprising yeast beta glucan, arabinan, xantham gum, gellen gum, yeast mannan, xylan, or any combination thereof.

Aspect A174: An oligosaccharide composition, comprising:
at least 35 wt. % (e.g., at least any of the following: 35, 40, 45, 50, 55, 60, 65, 70, 75, 80, 85, or 90 wt. %; optionally less than any of the following: 95, 90, 85, 80, 75, 70, 65, 60, 55, 50, 45, 40, or 35 wt. %; optionally wherein any of such values can be combined in any manner to form a range, such as 35-55 wt. %) fucose subunits, based on total weight of saccharide subunits.

Aspect A175: The oligosaccharide composition of aspect A174, or any preceding aspect, comprising at least 50 wt. % (e.g., at least any of the following: 50, 55, 60, 65, 70, 75, 80, 85, 90, 95, or 99 wt. %; optionally 100 wt. %; optionally less than any of the following: 100, 99, 95, 90, 85, 80, 75, 70, 65, 60, or 55 wt. %; optionally wherein any of such values can be combined in any manner to form a range, such as 55-90 wt. %) oligosaccharides on a dry basis having a degree of polymerization of between 3 and 50 (e.g., 3-5, 3-8, 3-10, 3-12, 3-15, 3-18, 3-20, 3-25, 3-30, 3-35, 3-40, 3-45, 5-8, 5-10, 5-12, 5-15, 5-18, 5-20, 5-25, 5-30, 5-35, 5-40, 5-45, 5-50, 10-15, 10-20, 10-25, 10-30, 10-35, 10-40, 10-45, 10-50, 15-20, 15-25, 15-30, 15-35, 15-40, 15-45, 15-50, 20-25, 20-30, 20-35, 20-40, 20-45, 20-50, 25-30, 25-35, 25-40, 25-45, 25-50, 30-35, 30-40, 30-45, 30-50, 35-40, 35-45, 35-50, 40-45, 40-50, or 45-50) monosaccharide subunits.

Aspect A176: The oligosaccharide composition of aspect A174 or A175, or any preceding aspect, comprising a dynamic viscosity at 25° C. of between 0.8 and 2.5 (e.g., 0.8-1, 0.8-1.2, 0.8-1.5, 0.8-1.7, 0.8-2, 0.8-2.2, 1-1.2, 1-1.5, 1-1.7, 1-2, 1-2.2, 1-2.5, 1.2-1.5, 1.2-1.7, 1.2-2, 1.2-2.2, 1.2-2.5, 1.5-1.7, 1.5-2, 1.5-2.2, 1.5-2.5, 1.7-2, 1.7-2.2, 1.7-2.5, 2-2.2, 2-2.5, or 2.2-2.5) mPa*s at a concentration of 100 mg of the oligosaccharide composition on a dry basis in 1 mL of water (i.e., per mL).

Aspect A177: The oligosaccharide composition of any one of aspects A174-A176, or any preceding aspect, comprising 12-25 wt. % (10-12, 10-15, 10-17, 10-20, 10-22, 10-25, 12-15, 12-17, 12-20, 12-22, 15-17, 15-20, 15-22, 15-25, 17-20, 17-22, 17-25, 20-22, 20-25, or 22-25 wt. %) xylose subunits, based on total weight of saccharide subunits.

Aspect A178: The oligosaccharide composition of any one of aspects A174-A177, or any preceding aspect, comprising 5-20 wt. % (e.g., 5-10, 5-15, 10-15, 10-20, or 15-20 wt. %) glucose subunits, based on total weight of saccharide subunits.

Aspect A179: The oligosaccharide composition of any one of aspects A174-A178, or any preceding aspect, comprising at least 3 wt. % (e.g., at least any of the following: 4, 5, 7, 10, 12, 15, 20, 25, or 30 wt. %; optionally less than any of the following: 35, 30, 25, 20, 15, 12, 10, 7, 5, or 4 wt. %; optionally wherein any of such values can be combined in any manner to form a range, such as 5-10 wt. %) 2-linked fucose subunits, based on total weight of saccharide subunits.

Aspect A180: The oligosaccharide composition of any one of aspects A174-A179, or any preceding aspect, comprising non-terminal xylose subunits, wherein at least 70% wt. % (e.g., at least any of the following: 75, 80, 85, 90, 95, 97, or 99 wt. %; optionally essentially all; optionally 100 wt. %; optionally less than any of the following: 100, 99, 97, 95, 90, 85, 80, or 75 wt. %; optionally where any such values can be combined in any manner to form a range, such as 97-99 wt. % or 90 wt. % to essentially all) of the non-terminal xylose subunits have at least one 4-linkage.

Aspect A181: The oligosaccharide composition of any one of aspects A174-A180, or any preceding aspect, comprising non-terminal glucose subunits, wherein at least 70% wt. % (e.g., at least any of the following: 75, 80, 85, 90, 95, 97, or 99 wt. %; optionally essentially all; optionally 100 wt. %; optionally less than any of the following: 100, 99, 97, 95, 90, 85, 80, or 75 wt. %; optionally where any such values can be combined in any manner to form a range, such as 85-99 wt. % or 90 wt. % to essentially all) of the non-terminal glucose subunits have at least one 3-linkage and/or at least one 6-linkage.

Aspect A182: The oligosaccharide composition of any one of aspects A174-A181, or any preceding aspect, comprising at least one of (e.g., at least two of, or at least three of): (a) at least three (e.g., at least 3, 4, 5, 6, 7, 8, or 9; optionally less than 10, 9, 8, 7, 6, 5, or 4; optionally wherein any of such values can be combined in any manner to form a range, such as 3-7) different isomers of 3Hex, (b) at least two (e.g., at least 3, 4, 5, 6, 7, 8, or 9; optionally less than 10, 9, 8, 7, 6, 5, 4, or 3; optionally wherein any of such values can be combined in any manner to form a range, such as 2-9) different isomers of 4Hex, (c) at least one (e.g., at least 2, 3, 4, 5, 6, 7, 8, or 9; optionally less than 10, 9, 8, 7, 6, 5, 4, 3, or 2; optionally wherein any of such values can be combined in any manner to form a range, such as 2-9) [different] isomer[s] of 5Hex, or (d) any combination thereof.

Aspect A183: The oligosaccharide composition of any one of aspects A174-A182, or any preceding aspect, wherein the oligosaccharide composition is within 30% (e.g., within 25%, within 20%, within 15%, within 10%, within 5%, within 3%, or within 1%) of at least 70% (e.g., 30-35, 30-40, 30-45, 30-50, 30-55, 30-60, 30-65, 35-40, 35-45, 35-50, 35-55, 35-60, 35-65, 35-70, 40-45, 40-50, 40-55, 40-60, 40-65, 40-70, 45-50, 45-55, 45-60, 45-65, 45-70, 50-55, 50-60, 50-65, 50-70, 55-60, 55-65, 55-70, 60-65, 60-70, or 65-70 wt. %) of the entries set forth in Table 33 on a numbers basis.

Aspect A184: The oligosaccharide composition of any one of aspects A174-A183, or any preceding aspect, wherein the oligosaccharide composition is CLX129.

Aspect A185: The oligosaccharide composition of any one of aspects A174-A184, or any preceding aspect, wherein the oligosaccharide composition is derived from a material comprising Sea Lettuce.

Aspect A186: The oligosaccharide composition of any one of aspects A174-A185, or any preceding aspect, wherein the oligosaccharide composition is derived from a material comprising fucoidan, xylan, or a combination thereof.

Aspect A187: An oligosaccharide composition, comprising:
at least 25 wt. % (e.g., at least any of the following: 30, 35, 45, 50, 55, 60, 65, 70, 75, or 80 wt. %; optionally less than any of the following: 85, 80, 75, 70, 65, 60, 55, 50, 45, 40, 35, or 30 wt. %; optionally wherein any of such values can be combined in any manner to form a range, such as 25-45 wt. %) rhamnose subunits, based on total weight of saccharide subunits.

Aspect A188: The oligosaccharide composition of aspect A187, or any preceding aspect, comprising at least 50 wt. % (e.g., at least any of the following: 50, 55, 60, 65, 70, 75, 80, 85, 90, 95, or 99 wt. %; optionally 100 wt. %; optionally less than any of the following: 100, 99, 95, 90, 85, 80, 75, 70, 65, 60, or 55 wt. %; optionally wherein any of such values can be combined in any manner to form a range, such as 55-90 wt. %) oligosaccharides on a dry basis having a degree of polymerization of between 3 and 50 (e.g., 3-5, 3-8, 3-10, 3-12, 3-15, 3-18, 3-20, 3-25, 3-30, 3-35, 3-40, 3-45, 5-8, 5-10, 5-12, 5-15, 5-18, 5-20, 5-25, 5-30, 5-35, 5-40, 5-45, 5-50, 10-15, 10-20, 10-25, 10-30, 10-35, 10-40, 10-45, 10-50, 15-20, 15-25, 15-30, 15-35, 15-40, 15-45, 15-50, 20-25, 20-30, 20-35, 20-40, 20-45, 20-50, 25-30, 25-35, 25-40, 25-45, 25-50, 30-35, 30-40, 30-45, 30-50, 35-40, 35-45, 35-50, 40-45, 40-50, or 45-50) monosaccharide subunits.

Aspect A189: The oligosaccharide composition of aspect A187 or A188, or any preceding aspect, comprising a dynamic viscosity at 25° C. of between 0.8 and 2.5 (e.g., 0.8-1, 0.8-1.2, 0.8-1.5, 0.8-1.7, 0.8-2, 0.8-2.2, 1-1.2, 1-1.5, 1-1.7, 1-2, 1-2.2, 1-2.5, 1.2-1.5, 1.2-1.7, 1.2-2, 1.2-2.2, 1.2-2.5, 1.5-1.7, 1.5-2, 1.5-2.2, 1.5-2.5, 1.7-2, 1.7-2.2, 1.7-2.5, 2-2.2, 2-2.5, or 2.2-2.5) mPa*s at a concentration of 100 mg of the oligosaccharide composition on a dry basis in 1 mL of water (i.e., per mL).

Aspect A190: The oligosaccharide composition of any one of aspects A187-A189, or any preceding aspect, comprising 30-50 wt. % (e.g., 30-35, 30-40, 30-45, 35-40, 35-45, 35-50, 40-45, 40-50, or 45-50 wt. %) galactose subunits, based on total weight of saccharide subunits.

Aspect A191: The oligosaccharide composition of any one of aspects A187-A190, or any preceding aspect, comprising a sum of galactose subunits, rhamnose subunits, and galacturonic acid subunits in an amount of at least 70 wt. % (e.g., at least any of the following: 75, 80, 85, 90, 95, 97, or 99 wt. %; optionally essentially all; optionally 100 wt. %; optionally less than any of the following: 100, 99, 97, 95, 90, 85, 80, or 75 wt. %; optionally where any such values can be combined in any manner to form a range, such as 85-99 wt. % or 90 wt. % to essentially all), based on total weight of saccharide subunits.

Aspect A192: The oligosaccharide composition of any one of aspects A187-A191, or any preceding aspect, comprising non-terminal galactose subunits, wherein at least 70 wt. % (e.g., at least any of the following: 75, 80, 85, 90, 95, 97, or 99 wt. %; optionally essentially all; optionally 100 wt. %; optionally less than any of the following: 100, 99, 97, 95, 90, 85, 80, or 75 wt. %; optionally where any such values can be combined in any manner to form a range, such as 85-99 wt. % or 90 wt. % to essentially all) of the non-terminal galactose subunits have at least one 4-linkage.

Aspect A193: The oligosaccharide composition of any one of aspects A187-A192, or any preceding aspect, comprising non-terminal rhamnose subunits, wherein at least 70% wt. % (e.g., at least any of the following: 75, 80, 85, 90, 95, 97, or 99 wt. %; optionally essentially all; optionally 100 wt. %; optionally less than any of the following: 100, 99, 97, 95, 90, 85, 80, or 75 wt. %; optionally where any such values can be combined in any manner to form a range, such as 85-99 wt. % or 90 wt. % to essentially all) of the non-terminal rhamnose subunits have at least one 2-linkage.

Aspect A194: The oligosaccharide composition of any preceding aspect.

Aspect A195: The oligosaccharide composition of any one of aspects A187-A194, or any preceding aspect, wherein the oligosaccharide composition is CLX107.

Aspect A196: The oligosaccharide composition of any one of aspects A187-A195, or any preceding aspect, wherein the oligosaccharide composition is derived from a material comprising Stercularia.

Aspect A197: The oligosaccharide composition of any one of aspects A187-A196, or any preceding aspect, wherein the oligosaccharide composition is derived from a material comprising Karaya gum.

Aspect A198: A formulation comprising the oligosaccharide composition of any one of aspects A112-A197, or any preceding aspect.

Aspect A199: A method for treating or preventing a disease, condition, disorder, and/or indication in a subject in need thereof, the method comprising:
administering to the subject the formulation comprising the oligosaccharide composition of any preceding aspect;
wherein the method treats or prevents the disease, condition, disorder, and/or indication in the subject.

Aspect A200: The method of aspect A199, or any preceding aspect, wherein the administering step comprising administering to the subject a therapeutically effective amount of the formulation.

Aspect A201: The method of aspect A199 or A200, or any preceding aspect, wherein the method modulates microbiota in the subject:
to produce at least one short chain fatty acids, optionally selected from butyrate, propionate, betahydroxybutyrate, lactate, acetate, or any combination thereof;
to produce at least one indole derivative;
to metabolize bile acid and/or bile salt;
or any combination thereof.

Aspect A202: The method of aspect A201, or any preceding aspect, wherein the microbiota are selected from:
Clostridium cluster I, optionally selected from Clostridium butyricum;
Clostridium cluster IV, optionally selected from Faecalibacterium prausnitzii;
Clostridium cluster XIVa, optionally selected from Roseburia spp, Eubacterium rectale, Eubacterium hallil, Blautia, Ruminococcus, Dorea, Coprococcus, Clostridium scindens, Clostridium hiranonis, Clostridium hylemonae, or any combination thereof;
Clostridium cluster XI, optionally selected from Clostridium sordelli;
Bacteroides thetaiotaomicron, Bacteroides ovatus, Bacteroides fragilis, Akkermansia Mucinophila, Bacteroides eggerthii, Ruminococcus bromii, Eubacterium dolichum, Veillonella parvula, or any combination thereof;
Bifidobacterium, optionally selected from Bifidobacterium longum, Bifidobacterium pseudocatenulatum, Bifidobacterium lactis, Bifidobacterium infantus, Bifidobacterium adolescentis, or Bifidobacterium animalis, or any combination thereof;
Lactobacilli, optionally selected from Lactobacillus lactis, Lactobacillus rhamnosus, or Lactobacillus plantarum, or any combination thereof;
Clostridium sporongenes or Peptosreptococcus, or a combination thereof; Eubacterium spp.;
or any combination thereof.

Aspect A203: The method of aspect A201 or A202, or any preceding aspect, wherein the method increases the abundance of the microbiota in the subject.

Aspect A204: The method of any one of aspects A201-A203, or any preceding aspect, wherein, in the subject, the method:
increases production of short chain fatty acids optionally comprising butyrate, propionate, betahydroxybutyrate, or any combination thereof;
increases production of indole derivatives optionally comprising indole-3-aldehyde, indole-3-lactate, indole-3-propionate, or any combination thereof;
metabolizes bile salts into bile acids, optionally wherein the bile acids comprise cholate, deoxycholate, lithocholate, chenodeoxycholate, ursodeoxycholate, 7-oxolithocholate, 7-oxo-deoxycholate, or any combination thereof; or
any combination thereof.

Aspect A205: The method of any one of aspects A201-A204, or any preceding aspect, wherein the method has an effect on the subject comprising:
lowering inflammation in the gastrointestinal tract of the subject;
lowering at least one of inflammatory gastrointestinal markers TNF-α, IL1-β, or IL-6, or any combination thereof, in the subject;
increasing at least one of anti-inflammatory gastrointestinal markers IL-10, TGF-beta, or IL-4, or any combination thereof, in the subject;
inhibiting histone deacetylases in the subject;
decreasing intestinal barrier permeability in the gastrointestinal tract of the subject;
increasing the expression of Muc2, occluding, claudin-4, or ZO-1 genes, or any combination thereof, in the gastrointestinal tract of the subject;
lowering A1c levels in the subject;
lowering post-prandial glucose response;
stimulates receptor FXR or TGR5, or a combination thereof, in the subject;
stimulating at least one of receptors GPR41, GPR43, GPR109A, TGR5, PSGR, PXR, or AhR, or any combination thereof, in the subject;
increasing the production of GLP-1 in the subject;
promoting motility in the subject;
inducing 5-HT biosynthesis in the subject; or
any combination thereof.

Aspect A206: The method of any one of aspects A199-A205, or any preceding aspect, wherein the disease, condition, disorder, and/or indication is selected from visceral pain, type 2 diabetes mellitus, atherosclerosis, epilepsy, IBD, Crohn's, NAFLD, Constipation, Celiac Disease, Diarrhea, IBS, or any combination thereof.

Aspect A207: The method of any one of aspects A199-A206, or any preceding aspect, wherein the oligosaccharide composition comprises CLX101, CLX101C, CLX102, CLX103, CLX105, CLX107, CLX108, CLX109, CLX110, CLX111, CLX112, CLX113, CLX114, CLX115, CLX115AL, CLX115-FC, CLX115a, CLX116, CLX117, CLX118, CLX119, CLX121, CLX122, CLX122DS, CLX122DSF, CLX123, CLX124, CLX125, CLX126, CLX127, CLX128, CLX129, CLX130, CLX131, CLX132, CLX133, or any combination thereof.

Aspect B1a: A method for modulating microbiota and/or their metabolic products, the method comprising contacting the microbiota with an oligosaccharide composition, wherein the oligosaccharide composition comprises, or collectively comprises, one or more oligosaccharides with xylose subunits.

Aspect B1b: A method for prevention or treatment of a cardiovascular disorder which comprises administering to a patient in need thereof an amount, or a therapeutically effective amount, of an oligosaccharide composition, wherein the oligosaccharide composition comprises, or collectively comprises, one or more oligosaccharides with xylose subunits.

Aspect B1c: A method for prevention or treatment of a renal disorder which comprises administering to a patient in need thereof an amount, or a therapeutically effective amount, of an oligosaccharide composition, wherein the oligosaccharide composition comprises, or collectively comprises, one or more oligosaccharides with xylose subunits.

Aspect B1d: A method for prevention or treatment of a nervous system disorder which comprises administering to a patient in need thereof an amount, or a therapeutically effective amount, of an oligosaccharide composition, wherein the oligosaccharide composition comprises, or collectively comprises, one or more oligosaccharides with xylose subunits.

Aspect B1e: A method for prevention or treatment of an immune system disorder which comprises administering to a patient in need thereof an amount, or a therapeutically effective amount, of an oligosaccharide composition, wherein the oligosaccharide composition comprises, or collectively comprises, one or more oligosaccharides with xylose subunits.

Aspect B1f: A method for prevention or treatment of inflammatory bowel disease which comprises administering to a patient in need thereof an amount, or a therapeutically effective amount, of an oligosaccharide composition, wherein the oligosaccharide composition comprises, or collectively comprises, one or more oligosaccharides with xylose subunits.

Aspect B2: The method of aspect B1 (i.e., any one of aspects B1a-B1f), or any preceding aspect, wherein the one or more oligosaccharides comprise at least 30% (e.g., at least 50%, at least 70%, or at least 90%; optionally 100%, less than 100%, less than 80%, less than 60%, or less than 40%, and such values can be combined in any manner to form a range, such as 30-100%) xylose subunits, as calculated by hydrolytic monosaccharide compositional analysis.

Aspect B3: The method of B1 or B2, or any preceding aspect, wherein the xylose subunits comprise 4-linkages and terminal linkages in a total amount of at least 10% (e.g., at least 30%, at least 50%, at least 70%, or at least 90%; optionally 100%, less than 100%, less than 80%, less than 60%, less than 40%, or less than 20%, and such values can be combined in any manner to form a range, such as 10-100%) as determined by glycosidic linkage composition analysis.

Aspect B4: The method of aspect B3, or any preceding aspect, wherein the ratio of 4-linkages to terminal linkages is about 6.5:1 to about 8.5:1 (e.g., 6.5:1 to 8:1, or 7.3 to 8.2), or about 7.6:1, as determined by glycosidic linkage composition analysis.

Aspect B5: The method of aspect B3 or B4, or any preceding aspect, wherein the 4-linkages are beta linked.

Aspect B6: The method of any one of aspects B1-B5, or any preceding aspect, wherein at least 30 wt. % (e.g., at least 50 wt. %, at least 70 wt. %, or at least 90 wt. %; optionally 100 wt. %, less than 100 wt. %, less than 80 wt. %, less than 60 wt. %, or less than 40 wt. % and such values can be combined in any manner to form a range, such as 30-60%) of the one or more oligosaccharides is between DP3 and DP 30 (e.g., a DP value of 3-4, 3-5, 3-6, 3-8, 3-10, 3-15, 3-20, 3-25, 3-30, 5-10, 5-15, 5-20, 5-25, 5-30, 8-10, 8-15, 8-20, 8-25, 8-30, 10-15, 10-20, 10-25, 10-30, 15-20, 15-25, 15-30, 20-25, 20-30, 25-30, 3-5, 5-8, 8-10, 10-15, 15-20, 20-25, or 25-30), based on total mass of the one or more oligosaccharides, as measured by oligosaccharide analysis.

Aspect B7: The method of any one of aspeccts B1-B6, or any preceding aspect, wherein the one or more oligosaccharides comprise one or more of the compounds set forth in Table D.

Aspect B8: The method of any one of aspeccts B1-B7, or any preceding aspect, wherein the one or more oligosaccharides comprise at least 30 wt. % (e.g., at least 50 wt. %, at least 70 wt. %, or at least 90 wt. %; optionally 100 wt. %, less than 100 wt. %, less than 80 wt. %, less than 60 wt. %, or less than 40 wt. % and such values can be combined in any manner to form a range, such as 30-100 wt. %) of the compounds set forth in Table D, based on total mass of the one or more oligosaccharides and total mass of the compounds set forth in Table D.

Aspect B9: The method of any one of aspeccts B1-B8, or any preceding aspect, wherein the one or more oligosaccharides comprise one or more of the 2D NMR correlations described in Table A for CLX 103.

Aspect B10: The method of any one of aspeccts B1-B9, or any preceding aspect, wherein the one or more oligosaccharides have an absolute viscosity of between about 0.6 and 1.2 mPa*s (e.g., 0.6-0.8, 0.6-1, 0.8-1, 1-2, or 0.8-1.2 mPa*s) at a concentration of 10 mg/ml in water at a temperature of 25° C.

Aspect B11: The method of any one of aspeccts B1-B10, or any preceding aspect, wherein the one or more oligosaccharides have an absolute viscosity of between about 0.7 and 3 mPa*s (e.g., 0.7-1.5, 0.7-2.5, 1.5-2.5, or 1-3 mPa*s) at a concentration of 50 mg/ml in water at a temperature of 25° C.

Aspect B12: The method of any one of aspeccts B1-B11, or any preceding aspect, wherein the one or more oligosaccharides have an absolute viscosity of between about 0.7 and 8 mPa*s (e.g., 0.7-2, 0.7-4, 0.7-6, 1-3, 1-5, 1-8, 2-4, 2-6, 2-8, 4-6, 4-8, 6-8, 1-4, 4-6, or 6-8) at a concentration of 100 mg/ml in water at a temperature of 25° C.

Aspect B13: The method of any one of aspeccts B1-B12, or any preceding aspect, wherein the method enhances microbial production of at least one of short chain fatty acids, propionate, acetate, glycerate, or any combination thereof.

Aspect B14: The method of any one of aspeccts B1-B13, or any preceding aspect, wherein the method increases abundance of at least one of Bifidobacteria, Bifidobacterium pseudocatenulatum, Bifidobacterium animalis, Bacteroides, Bacteroides ovatus, Firmicutes, Clostridium butyricum, Ruminococcus, Ruminococcus gnavus, Ruminococcus torque, Blautia, Roseburia, Faecalibacterium, or any combination thereof.

Aspect B15: The method of any one of aspeccts B1-B14, or any preceding aspect, wherein the method slows microbial utilization of choline.

Aspect B16: The method of any one of aspeccts B1-B15, or any preceding aspect, wherein the method enhances microbial production of at least one of nicotinic acid, pantothenic acid, isoleucine, valine, gamma aminobutyric acid, ornithine, or any combination thereof.

Aspect B17: The method of any one of aspeccts B1-B16, or any preceding aspect, wherein the method decreases microbial production of at least one of cadaverine, putrescine, or a combination thereof.

Aspect B18: The method of any one of aspeccts B1-B17, or any preceding aspect, wherein the oligosaccharide composition further comprises at least one microorganism.

Aspect B19: The method of aspect B18, or any preceding aspect, wherein the at least one microorganism is modulated by the oligosaccharide composition.

Aspect B20: The method of aspect B18 or B19, or any preceding aspect, wherein the at least one microorganism is at least one of Bifidobacteria, Bifidobacterium pseudocatenulatum, Bifidobacterium animalis, Bacteroides, Bacteroides ovatus, Firmicutes, Clostridium butyricum, Ruminococcus, Ruminococcus gnavus, Ruminococcus torque, Blautia, Roseburia, Faecalibacterium, or any combination thereof.

Aspect C1a: A method for modulating microbiota and/or their metabolic products, the method comprising contacting the microbiota with an oligosaccharide composition, wherein the oligosaccharide composition comprises, or collectively comprises, one or more oligosaccharides with glucose subunits.

Aspect C1b: A method for prevention or treatment of a cardiovascular disorder which comprises administering to a patient in need thereof an amount, or a therapeutically effective amount, of an oligosaccharide composition, wherein the oligosaccharide composition comprises, or collectively comprises, one or more oligosaccharides with glucose subunits.

Aspect C1c: A method for prevention or treatment of a renal disorder which comprises administering to a patient in need thereof an amount, or a therapeutically effective amount, of an oligosaccharide composition, wherein the oligosaccharide composition comprises, or collectively comprises, one or more oligosaccharides with glucose subunits.

Aspect C1d: A method for prevention or treatment of a nervous system disorder which comprises administering to a patient in need thereof an amount, or a therapeutically effective amount, of an oligosaccharide composition, wherein the oligosaccharide composition comprises, or collectively comprises, one or more oligosaccharides with glucose subunits.

Aspect C1e: A method for prevention or treatment of an immune system disorder which comprises administering to a patient in need thereof an amount, or a therapeutically effective amount, of an oligosaccharide composition, wherein the oligosaccharide composition comprises, or collectively comprises, one or more oligosaccharides with glucose subunits.

Aspect C1f: A method for prevention or treatment of inflammatory bowel disease which comprises administering to a patient in need thereof an amount, or a therapeutically effective amount, of an oligosaccharide composition, wherein the oligosaccharide composition comprises, or collectively comprises, one or more oligosaccharides with glucose subunits.

Aspect C2: The method of aspect C1 (i.e., any one of aspects C1a-C1f), or any preceding aspect, wherein at least 30 wt. % (e.g., at least 50 wt. %, at least 70 wt. %, or at least 90 wt. %; optionally 100 wt. %, less than 100 wt. %, less than 80 wt. %, less than 60 wt. %, or less than 40 wt. % and such values can be combined in any manner to form a range, such as 30-80 wt. %) of the one or more oligosaccharides is between DP3 and DP 30 (e.g., a DP value of 3-4, 3-5, 3-6, 3-8, 3-10, 3-15, 3-20, 3-25, 3-30, 5-10, 5-15, 5-20, 5-25, 5-30, 8-10, 8-15, 8-20, 8-25, 8-30, 10-15, 10-20, 10-25, 10-30, 15-20, 15-25, 15-30, 20-25, 20-30, 25-30, 3-5, 5-8, 8-10, 10-15, 15-20, 20-25, or 25-30), based on total mass of the one or more oligosaccharides, as measured by oligosaccharide analysis.

Aspect C3: The method of aspect C1 or C2, or any preceding aspect, wherein the one or more oligosaccharides comprise at least 10% (e.g., at least 30%, at least 50%, at least 70%, or at least 90%; optionally 100%, less than 100%, less than 80%, less than 60%, less than 40%, or less than 20%, and such values can be combined in any manner to form a range, such as 10-80%) glucose subunits, as calculated by hydrolytic monosaccharide compositional analysis.

Aspect C4: The method of any one of aspects C1-C3, or any preceding aspect, wherein the glucose subunits comprise 3-linkages and terminal linkages in a total amount of at least 10% (e.g., at least 30%, at least 50%, at least 70%, or at least 90%; optionally 100%, less than 100%, less than 80%, less than 60%, less than 40%, or less than 20%, and such values can be combined in any manner to form a range, such as 10-80%), as determined by glycosidic linkage composition analysis.

Aspect C5: The method of aspect C4, or any preceding aspect, wherein the ratio of 3-linkages to terminal linkages is about 7.5:1 to 9.5:1 (e.g., 7.5:1 to 8.5:1, or 8:1 to 9:1, or 8:1-9.5:1), or about 8.5:1, as determined by glycosidic linkage composition analysis.

Aspect C6: The method of aspect C5 or C5, or any preceding aspect, wherein the 3-linkages are beta linked.

Aspect C7: The method of any one of aspects C1-C6, or any preceding aspect, wherein the one or more oligosaccharides comprise one or more of the compounds set forth in Table B.

Aspect C8: The method of any one of aspects C1-C7, or any preceding aspect, wherein the one or more oligosaccharides comprise at least 30 wt. % (e.g., at least 50 wt. %, at least 70 wt. % or at least 90 wt. %; optionally optionally 100 wt. %, less than 100 wt. %, less than 80 wt. %, less than 60 wt. %, or less than 40 wt. % and such values can be combined in any manner to form a range, such as 30-40 wt. %) of the compounds set forth in Table B, based on total mass of the one or more oligosaccharides and total mass of the compounds set forth in Table B.

Aspect C9: The method of any one of aspects C1-C8, or any preceding aspect, wherein the one or more oligosaccharides comprise one or more of the 2D NMR correlations described in Table A for CLX 101.

Aspect C10: The method of any one of aspects C1-C9, or any preceding aspect, wherein the one or more oligosaccharides have an absolute viscosity of between about 0.6 and 1.2 mPa*s (e.g., 0.6-0.8, 0.6-1, 0.8-1, 1-2, or 0.8-1.2 mPa*s) at a concentration of 10 mg/ml in water at a temperature of 25° C.

Aspect C11: The method of any one of aspects C1-C10, or any preceding aspect, wherein the one or more oligosaccharides have an absolute viscosity of between about 0.7 and 3 mPa*s (e.g., 0.7-1.5, 0.7-2.5, 1.5-2.5, or 1-3 mPa*s) at a concentration of 50 mg/ml in water at a temperature of 25° C.

Aspect C12: The method of any one of aspects C1-C11, or any preceding aspect, wherein the one or more oligosaccharides have an absolute viscosity of between about 0.7 and 8 mPa*s (e.g., 0.7-2, 0.7-4, 0.7-6, 1-3, 1-5, 1-8, 2-4, 2-6, 2-8, 4-6, 4-8, 6-8, 1-4, 4-6, or 6-8) at a concentration of 100 mg/ml in water at a temperature of 25° C.

Aspect C13: The method of any one of aspects C1-C12, or any preceding aspect, wherein the method enhances microbial production of at least one of short chain fatty acids, butyrate, lactate, 3-hydroxybutyrate, nicotinic acid, isoleucine, gamma aminobutyric acid, glutamate, ornithine, or any combination thereof.

Aspect C14: The method of any one of aspects C1-C13, or any preceding aspect, wherein the method increases abundance of at least one of Lactobacillus crispatus, Lactobacillus rhamnosus, Bifidobacteria, Bifidobacterium pseudocatenulatum, Bifidobacterium longum subsp. longum, Bifidobacterium animalis, Firmicutes, Clostridium butyricum, Ruminococcus, Ruminococcus gnavus, Blautia, Roseburia, or any combination thereof.

Aspect C15: The method of any one of aspects C1-C14, or any preceding aspect, wherein the method decreases microbial utilization of methionine.

Aspect C16: The method of any one of aspects C1-C15, or any preceding aspect, wherein the method decreases microbial production of at least one of histamine, putrescine, or a combination thereof.

Aspect C17: The method of any one of aspects C1-C16, or any preceding aspect, wherein the oligosaccharide composition further comprises at least one microorganism.

Aspect C18: The method of aspect C17, or any preceding aspect, wherein the at least one microorganism is modulated by the oligosaccharide composition.

Aspect C19: The method of aspect C17 or C18, or any preceding aspect, wherein the at least one microorganism is at least one of Lactobacillus crispatus, Lactobacillus rhamnosus, Bifidobacteria, Bifidobacterium pseudocatenulatum, Bifidobacterium longum subsp. longum, Bifidobacterium animalis, Firmicutes, Clostridium butyricum, Ruminococcus, Ruminococcus gnavus, Blautia, Roseburia, or any combination thereof.

Aspect D1a: A method for modulating microbiota and/or their metabolic products, the method comprising contacting the microbiota with an oligosaccharide composition, wherein the oligosaccharide composition comprises, or collectively comprises, one or more oligosaccharides with glucose subunits.

Aspect D1b: A method for prevention or treatment of a cardiovascular disorder which comprises administering to a patient in need thereof an amount, or a therapeutically effective amount, of an oligosaccharide composition, wherein the oligosaccharide composition comprises, or collectively comprises, one or more oligosaccharides with glucose subunits.

Aspect D1c: A method for prevention or treatment of a renal disorder which comprises administering to a patient in need thereof an amount, or a therapeutically effective amount, of an oligosaccharide composition, wherein the oligosaccharide composition comprises, or collectively comprises, one or more oligosaccharides with glucose subunits.

Aspect D1d: A method for prevention or treatment of a nervous system disorder which comprises administering to a patient in need thereof an amount, or a therapeutically effective amount, of an oligosaccharide composition, wherein the oligosaccharide composition comprises, or collectively comprises, one or more oligosaccharides with glucose subunits.

Aspect D1e: A method for prevention or treatment of an immune system disorder which comprises administering to a patient in need thereof an amount, or a therapeutically effective amount, of an oligosaccharide composition, wherein the oligosaccharide composition comprises, or collectively comprises, one or more oligosaccharides with glucose subunits.

Aspect D1f: A method for prevention or treatment of inflammatory bowel disease which comprises administering to a patient in need thereof an amount, or a therapeutically effective amount, of an oligosaccharide composition, wherein the oligosaccharide composition comprises, or collectively comprises, one or more oligosaccharides with glucose subunits.

Aspect D2: The method of aspect D1 (i.e., any one of aspects D1a-D1f), or any preceding aspect, wherein at least 30 wt. % (e.g., at least 50 wt. %, at least 70 wt. %, or at least 90 wt. %; optionally 100 wt. %, less than 100 wt. %, less than 80 wt. %, less than 60 wt. %, or less than 40 wt. % and such values can be combined in any manner to form a range, such as 30-60 wt. %) of the one or more oligosaccharides is between DP3 and DP 30 (e.g., a DP value of 3-4, 3-5, 3-6, 3-8, 3-10, 3-15, 3-20, 3-25, 3-30, 5-10, 5-15, 5-20, 5-25, 5-30, 8-10, 8-15, 8-20, 8-25, 8-30, 10-15, 10-20, 10-25, 10-30, 15-20, 15-25, 15-30, 20-25, 20-30, 25-30, 3-5, 5-8, 8-10, 10-15, 15-20, 20-25, or 25-30), based on total mass of the one or more oligosaccharides, as measured by oligosaccharide analysis.

Aspect D3: The method of aspect D1 or D2, or any preceding aspect, wherein the one or more oligosaccharides comprise at least 10% (e.g., at least 30%, at least 50%, at least 70%, or at least 90%; optionally 100%, less than 100%, less than 80%, less than 60%, less than 40%, or less than 20% and such values can be combined in any manner to form a range, such as 50-80%) glucose subunits as calculated by hydrolytic monosaccharide compositional analysis.

Aspect D4: The method of any one of aspects D1-D3, or any preceding aspect, wherein the glucose subunits comprise 3-linkages, 4-linkages and terminal linkages in a total amount of at least 10% (e.g., at least 30%, at least 50%, at least 70%, or at least 90%; optionally 100%, less than 100%, less than 80%, less than 60%, less than 40%, or less than 20% and such values can be combined in any manner to form a range, such as 30-60%), as determined by glycosidic linkage composition analysis.

Aspect D5: The method of any one of aspects D1-D4, or any preceding aspect, wherein the glucose subunits comprise a ratio of 3-linkages to 4-linkages to terminal linkages of about 0.4-0.7:1-2:1, about 0.55:1.6:1, about 1-3:4-6:1, or about 1.8:5:1, or any combination thereof, as determined by glycosidic linkage composition analysis.

Aspect D6: The method of aspect D4 or D5, or any preceding aspect, wherein the ratio of 3-linkages:4-linkages is between 0.2:1 to 8:1 (e.g., 0.2:1 to 1:1, 0.2:1 to 4:1, 0.2:1 to 6:1, 0.5:1 to 3:1, 0.5:1 to 6:1, 1:1 to 8:1, 2:1 to 4:1, 3:1 to 6:1, or 5:1 to 8:1), as determined by glycosidic linkage composition analysis.

Aspect D7: The method of any one of aspects D4-D6, or any preceding aspect, wherein the ratio of 3-linkages:4-linkages is about 0.35:1 (e.g., 0.2:1 to 0.5:1), as determined by glycosidic linkage composition analysis.

Aspect D8: The method of any one of aspects D4-D7, or any preceding aspect, wherein the ratio of 4-linkages to terminal linkages is about 6.5:1 to about 8.5:1 (e.g., 6.5:1 to 8:1, or 7.3 to 8.2), or about 7.6:1.

Aspect D9: The method of any one of aspects D4-D8, or any preceding aspect, wherein the 3-linkages are beta linked.

Aspect D10: The method of any one of aspects D4-D9, or any preceding aspect, wherein the 4-linkages are beta linked

Aspect D11: The method of any one of aspects D1-D10, or any preceding aspect, wherein the one or more oligosaccharides comprise one or more of the compounds set forth in Table J and Table M.

Aspect D12: The method of any one of aspects D1-D11, or any preceding aspect, wherein the one or more oligosaccharides comprise at least 30 wt. % (e.g., at least 50 wt. %, at least 70 wt. %, or at least 90 wt. %; optionally 100 wt. %, less than 100 wt. %, less than 80 wt. %, less than 60 wt. %, or less than 40 wt. %, and such values can be combined in any manner to form a range, such as 30-80 wt. %) of the compounds set forth in Table J, based on total mass of the one or more oligosaccharides and total mass of the compounds set forth in Table J and Table M.

Aspect D13: The method of any one of aspects D1-D12, or any preceding aspect, wherein the one or more oligosaccharides comprise one or more of the 2D NMR correlations described in Table A for CLX 112.

Aspect D14: The method of any one of aspects D1-D13, or any preceding aspect, wherein the one or more oligosaccharides have an absolute viscosity of between about 0.6 and 1.2 mPa*s (e.g., 0.6-0.8, 0.6-1, 0.8-1, 1-2, or 0.8-1.2 mPa*s) at a concentration of 10 mg/ml in water at a temperature of 25° C.

Aspect D15: The method of any one of aspects D1-D14, or any preceding aspect, wherein the one or more oligosaccharides have an absolute viscosity of between about 0.7 and 3 mPa*s (e.g., 0.7-1.5, 0.7-2.5, 1.5-2.5, or 1-3 mPa*s) at a concentration of 50 mg/ml in water at a temperature of 25° C.

Aspect D16: The method of any one of aspects D1-D15, or any preceding aspect, wherein the one or more oligosaccharides have an absolute viscosity of between about 0.7 and 8 mPa*s (e.g., 0.7-2, 0.7-4, 0.7-6, 1-3, 1-5, 1-8, 2-4, 2-6, 2-8, 4-6, 4-8, 6-8, 1-4, 4-6, or 6-8) at a concentration of 100 mg/ml in water at a temperature of 25° C.

Aspect D17: The method of any one of aspects D1-D16, or any preceding aspect, wherein the method enhances microbial production of at least one of short chain fatty acids, butyrate, lactate, acetate, 3-hydroxybutyrate, valine, gamma aminobutyric acid, ornithine, or any combination thereof.

Aspect D18: The method of any one of aspects D1-D17, or any preceding aspect, wherein the method increases abundance of at least one of Lactobacillus crispatus, Lactobacillus rhamnosus, Bifidobacteria, Bifidobacterium longum subsp. infantis, Bifidobacterium animalis, Firmicutes, Clostridium butyricum, Ruminococcus, Ruminococcus gnavus, Blautia, Roseburia, Faecalibacterium, or any combination thereof.

Aspect D19: The method of any one of aspects D1-D18, or any preceding aspect, wherein the method slows microbial utilization of choline.

Aspect D20: The method of any one of aspects D1-D19, or any preceding aspect, wherein the method decreases microbial utilization of methionine.

Aspect D21: The method of any one of aspects D1-D2O, or any preceding aspect, wherein the method decreases microbial production of putrescine.

Aspect D22: The method of any one of aspects D1-D21, or any preceding aspect, wherein the oligosaccharide composition further comprises at least one microorganism.

Aspect D23: The method of aspect D22, or any preceding aspect, wherein the at least one microorganism is modulated by the oligosaccharide composition.

Aspect D24: The method of aspect D22 or D23, or any preceding aspect, wherein the at least one microorganism is at least one of Lactobacillus crispatus, Lactobacillus rhamnosus, Bifidobacteria, Bifidobacterium longum subsp. infantis, Bifidobacterium animalis, Firmicutes, Clostridium butyricum, Ruminococcus, Ruminococcus gnavus, Blautia, Roseburia, Faecalibacterium, or any combination thereof.

Aspect E1a: A method for modulating microbiota and/or their metabolic products, the method comprising contacting the microbiota with an oligosaccharide composition, wherein the oligosaccharide composition comprises, or collectively comprises, one or more oligosaccharides with glucose subunits.

Aspect E1b: A method for prevention or treatment of a cardiovascular disorder which comprises administering to a patient in need thereof an amount, or a therapeutically effective amount, of an oligosaccharide composition, wherein the oligosaccharide composition comprises, or collectively comprises, one or more oligosaccharides with glucose subunits.

Aspect E1c: A method for prevention or treatment of a renal disorder which comprises administering to a patient in need thereof an amount, or a therapeutically effective amount, of an oligosaccharide composition, wherein the oligosaccharide composition comprises, or collectively comprises, one or more oligosaccharides with glucose subunits.

Aspect E1d: A method for prevention or treatment of a nervous system disorder which comprises administering to a patient in need thereof an amount, or a therapeutically effective amount, of an oligosaccharide composition, wherein the oligosaccharide composition comprises, or collectively comprises, one or more oligosaccharides with glucose subunits.

Aspect E1e: A method for prevention or treatment of an immune system disorder which comprises administering to a patient in need thereof an amount, or a therapeutically effective amount, of an oligosaccharide composition, wherein the oligosaccharide composition comprises, or collectively comprises, one or more oligosaccharides with glucose subunits.

Aspect E1f: A method for prevention or treatment of inflammatory bowel disease which comprises administering to a patient in need thereof an amount, or a therapeutically effective amount, of an oligosaccharide composition, wherein the oligosaccharide composition comprises, or collectively comprises, one or more oligosaccharides with glucose subunits.

Aspect E2: The method of aspect E1 (i.e., any one of aspect E1a-E1f), or any preceding aspect, wherein at least 30 wt. % (e.g., at least 50 wt. %, at least 70 wt. %, or at least 90 wt. %; optionally 100 wt. %, less than 100 wt. %, less than 80 wt. %, less than 60 wt. %, or less than 40 wt. % and such values can be combined in any manner to form a range, such as 30-60 wt. %) of the one or more oligosaccharides is between DP3 and DP 30 (e.g., a DP value of 3-4, 3-5, 3-6, 3-8, 3-10, 3-15, 3-20, 3-25, 3-30, 5-10, 5-15, 5-20, 5-25, 5-30, 8-10, 8-15, 8-20, 8-25, 8-30, 10-15, 10-20, 10-25, 10-30, 15-20, 15-25, 15-30, 20-25, 20-30, 25-30, 3-5, 5-8, 8-10, 10-15, 15-20, 20-25, or 25-30), based on total mass of the one or more oligosaccharides, as measured by oligosaccharide analysis.

Aspect E3: The method of aspect E1 or E2, or any preceding aspect, wherein the one or more oligosaccharides contain at least 10% (e.g., at least 30%, at least 50%, at least 70%, or at least 90%; optionally 100%, less than 100%, less than 80%, less than 60%, less than 40%, or less than 20%, and such values can be combined in any manner to form a range, such as 10-80%) glucose subunits, as calculated by hydrolytic monosaccharide compositional analysis.

Aspect E4: The method of any one of aspect E1-E3, or any preceding aspect, wherein the one or more oligosaccharides further comprise galactose subunits, mannose subunits, or a combination thereof.

Aspect E5: The method of aspect E4, or any preceding aspect, wherein the galactose:glucose ratio is between 0.1:1 to 2:1 (e.g., 0.1:1 to 0.8:1, 0.8:1 to 1.5:1, 1.5:1 to 2:1, 0.1:1 to 1:1, or 1:1 to 2:1), as calculated by hydrolytic monosaccharide compositional analysis.

Aspect E6: The method of aspect E4 or E5, or any preceding aspect, wherein the mannose:glucose ratio is about 0.1:1 to 2:1 (e.g., 0.1:1 to 0.8:1, 0.8:1 to 1.5:1, 1.5:1 to 2:1, 0.1:1 to 1:1, or 1:1 to 2:1), as calculated by hydrolytic monosaccharide compositional analysis.

Aspect E7: The method of any one of aspect E1-E6, or any preceding aspect, wherein the glucose subunits comprise 3-linkages, 4-linkages, and terminal linkages in a total amount of at least 30% (e.g., at least 50%, at least 70%, or at least 90%; optionally 100%, less than 100%, less than 80%, less than 60%, or less than 40% and such values can be combined in any manner to form a range, such as 50-80%), as determined by glycosidic linkage composition analysis.

Aspect E8: The method of aspect E7, or any preceding aspect, wherein the glucose subunits comprise a ratio of 3-linkages to 4-linkages to terminal linkages of about 1-3:0.5-2:1, or about 1.9:1.1:1, as determined by glycosidic linkage composition analysis.

Aspect E9: The method of aspect E7 or E8, or any preceding aspect, wherein the ratio of 3-linkages:4-linkages is between 0.1:1 to 8:1 (e.g., 0.1: to 2:1, 1:1 to 3:1, 2:1 to 4:1, 3:1 to 5:1, 4:1 to 6:1, 5:1 to 7:1, 6:1 to 8:1, 0.5:1 to 4:1, or 4:1 to 8:1), as determined by glycosidic linkage composition analysis.

Aspect E10: The method of aspect E9, or any preceding aspect, wherein the ratio of 3-linkages:4-linkages is about 0.6:1 (e.g., 0.2:1 to 0.8:1), as determined by glycosidic linkage composition analysis.

Aspect E11: The method of any one of aspects E4-E10, or any preceding aspect, wherein the mannose subunits comprise 2-linkages, 3-linkages, and terminal linkages in a total amount of at least 10% (e.g., at least 30%, at least 50%, at least 70%, or at least 90%; optionally 100%, less than 100%, less than 80%, less than 60%, less than 40%, or less than 20%, and such values can be combined in any manner to form a range, such as 10-100%), as determined by glycosidic linkage composition analysis.

Aspect E12: The method of any one of aspects E7-E11, or any preceding aspect, wherein the glucose 3-linkages are beta linked.

Aspect E13: The method of any one of aspects E7-E12, or any preceding aspect, wherein the glucose 4-linkages are beta linked

Aspect E14: The method of any one of aspects E1-E13, or any preceding aspect, wherein the one or more oligosaccharides comprise one or more of the compounds set forth in Table H.

Aspect E15: The method of any one of aspects E1-E14, or any preceding aspect, wherein the one or more oligosaccharides comprise at least 30 wt. % (e.g., at least 50 wt. %, at least 70 wt. %, or at least 90 wt. %; optionally 100 wt. %, less than 100 wt. %, less than 80 wt. %, less than 60 wt. %, or less than 40 wt. % and such values can be combined in any manner to form a range, such as 70-80 wt. %) of the compounds set forth in Table H, based on total mass of the one or more oligosaccharides and total mass of the compounds set forth in Table H.

Aspect E16: The method of any one of aspects E1-E15, or any preceding aspect, wherein the one or more oligosaccharides comprise one or more of the 2D NMR correlations described in Table A for CLX 110.

Aspect E17: The method of any one of aspects E1-E16, or any preceding aspect, wherein the one or more oligosaccharides have an absolute viscosity of between about 0.6 and 1.2 mPa*s (e.g., 0.6-0.8, 0.6-1, 0.8-1, 1-2, or 0.8-1.2 mPa*s) at a concentration of 10 mg/ml in water at a temperature of 25° C.

Aspect E18: The method of any one of aspects E1-E17, or any preceding aspect, wherein the one or more oligosaccharides have an absolute viscosity of between about 0.7 and 3 mPa*s (e.g., 0.7-1.5, 0.7-2.5, 1.5-2.5, or 1-3 mPa*s) at a concentration of 50 mg/ml in water at a temperature of 25° C.

Aspect E19: The method of any one of aspects E1-E18, or any preceding aspect, wherein the one or more oligosaccharides have an absolute viscosity of between about 0.7 and 8 mPa*s (e.g., 0.7-2, 0.7-4, 0.7-6, 1-3, 1-5, 1-8, 2-4, 2-6, 2-8, 4-6, 4-8, 6-8, 1-4, 4-6, or 6-8) at a concentration of 100 mg/ml in water at a temperature of 25° C.

Aspect E20: The method of any one of aspects E1-E19, or any preceding aspect, wherein the method enhances microbial production of at least one of short chain fatty acids, butyrate, propionate, acetate, isoleucine, gamma aminobutyric acid, glutamate, ornithine, or any combination thereof.

Aspect E21: The method of any one of aspects E1-E20, or any preceding aspect, wherein the method increases abundance of at least one of Lactobacillus crispatus, Lactobacillus rhamnosus, Bifidobacterium pseudocatenulatum, Bacteroides, Bacteroides ovatus, Firmicutes, Clostridium butyricum, Ruminococcus, Ruminococcus gnavus, Blautia, Roseburia, Faecalibacteria, or any combination thereof.

Aspect E22: The method of any one of aspects E1-E21, or any preceding aspect, wherein the method decreases microbial utilization of methionine.

Aspect E23: The method of any one of aspects E1-E22, or any preceding aspect, wherein the method decreases microbial production of putrescine.

Aspect E24: The method of any one of aspects E1-E23, or any preceding aspect, wherein the oligosaccharide composition further comprises at least one microorganism.

Aspect E25: The method of aspect E24, or any preceding aspect, wherein the at least one microorganism is modulated by the oligosaccharide composition.

Aspect E26: The method of aspect E24 or E25, or any preceding aspect, wherein the at least one microorganism is at least one of Lactobacillus crispatus, Lactobacillus rhamnosus, Bifidobacterium pseudocatenatum, Bacteroides, Bacteroides ovatus, Firmicutes, Clostridium butyricum, Ruminococcus, Ruminococcus gnavus, Blautia, Roseburia, Faecalibacteria, or any combination thereof.

Aspect F1a: A method for modulating microbiota and/or their metabolic products, the method comprising contacting the microbiota with an oligosaccharide composition, wherein the oligosaccharide composition comprises, or collectively comprises, one or more oligosaccharides with glucose and mannose subunits.

Aspect F1b: A method for prevention or treatment of a cardiovascular disorder which comprises administering to a patient in need thereof an amount, or a therapeutically effective amount, of an oligosaccharide composition, wherein the oligosaccharide composition comprises, or collectively comprises, one or more oligosaccharides with glucose and mannose subunits.

Aspect F1c: A method for prevention or treatment of a renal disorder which comprises administering to a patient in need thereof an amount, or a therapeutically effective amount, of an oligosaccharide composition, wherein the oligosaccharide composition comprises, or collectively comprises, one or more oligosaccharides with glucose and mannose subunits.

Aspect F1d: A method for prevention or treatment of a nervous system disorder which comprises administering to a patient in need thereof an amount, or a therapeutically effective amount, of an oligosaccharide composition, wherein the oligosaccharide composition comprises, or collectively comprises, one or more oligosaccharides with glucose and mannose subunits.

Aspect F1e: A method for prevention or treatment of an immune system disorder which comprises administering to a patient in need thereof an amount, or a therapeutically effective amount, of an oligosaccharide composition, wherein the oligosaccharide composition comprises, or collectively comprises, one or more oligosaccharides with glucose and mannose subunits.

Aspect F2: The method of aspect F1 (i.e., any one of aspects F1a-F1e), or any preceding aspect, wherein at least 30 wt. % (e.g., at least 50 wt. %, at least 70 wt. %, or at least 90 wt. %; optionally 100 wt. %, less than 100 wt. %, less than 80 wt. %, less than 60 wt. %, or less than 40 wt. % and such values can be combined in any manner to form a range, such as 30-100 wt. %) of the one or more oligosaccharides is between DP3 and DP 30 (e.g., a DP value of 3-4, 3-5, 3-6, 3-8, 3-10, 3-15, 3-20, 3-25, 3-30, 5-10, 5-15, 5-20, 5-25, 5-30, 8-10, 8-15, 8-20, 8-25, 8-30, 10-15, 10-20, 10-25, 10-30, 15-20, 15-25, 15-30, 20-25, 20-30, 25-30, 3-5, 5-8, 8-10, 10-15, 15-20, 20-25, or 25-30), based on total mass of the one or more oligosaccharides, as measured by oligosaccharide analysis.

Aspect F3: The method of aspect F1 or F2, or any preceding aspect, wherein the one or more oligosaccharides contain at least 10% (e.g., at least 30%, at least 50%, at least 70%, or at least 90%; optionally 100%, less than 100%, less than 80%, less than 60%, less than 40%, or less than 20%, and such values can be combined in any manner to form a range, such as 10-100%) glucose and mannose subunits, as calculated by hydrolytic monosaccharide compositional analysis.

Aspect F4: The method of any one of aspects F1-F3, or any preceding aspect, wherein the mannose:glucose ratio is between 0.2:1 and 8:1 (e.g., 0.2:1 to 3:1, 0.8:1 to 2.5:1, 0.5:1 to 4:1, 2:1 to 6:1, or 4:1 to 8:1), as calculated by hydrolytic monosaccharide compositional analysis.

Aspect F5: The method of any one of aspects F1-F4, or any preceding aspect, wherein the mannose:glucose ratio is about 1.6:1 (e.g., 1:1 to 2:1), as calculated by hydrolytic monosaccharide compositional analysis.

Aspect F6: The method of any one of aspects F1-F5, or any preceding aspect, wherein the glucose subunits comprise 4-linkages and terminal linkages in a total amount of at least 10% (e.g., at least 30%, at least 50%, at least 70%, or at least 90%; optionally 100%, less than 100%, less than 80%, less than 60%, less than 40%, or less than 20%, and such values can be combined in any manner to form a range, such as 60-100%), as determined by glycosidic linkage composition analysis.

Aspect F7: The method of any one of aspects F1-F6, or any preceding aspect, wherein the glucose subunits comprise 4-linkages and terminal linkages in a ratio of about 3-5:1, or about 4:1, as determined by glycosidic linkage composition analysis.

Aspect F8: The method of any one of aspects F1-F7, or any preceding aspect, wherein the mannose subunits comprise 4-linkages and terminal linkages in a ratio of about 1-3:1, or about 1.7:1, as determined by glycosidic linkage composition analysis.

Aspect F9: The method of any one of aspects F1-F8, or any preceding aspect, wherein the mannose subunits comprise 4-linkages and terminal linkages in a total amount of at least 30% (e.g., at least 50%, at least 70%, or at least 90%; optionally 100%, less than 100%, less than 80%, less than 60%, or less than 40% and such values can be combined in any manner to form a range, such as 30-60%) as determined by glycosidic linkage composition analysis.

Aspect F10: The method of any one of aspects F6-F9, or any preceding aspect, wherein the glucose 4-linkages are beta linked.

Aspect F11: The method of any one of aspects F8-F10, or any preceding aspect, wherein the mannose 4-linkages are beta linked

Aspect F12: The method of any one of aspects F1-F11, or any preceding aspect, wherein the one or more oligosaccharides comprise one or more compounds set forth in Table C.

Aspect F13: The method of any one of aspects F1-F12, or any preceding aspect, wherein the one or more oligosaccharides comprise at least 30 wt. % (e.g., at least 50 wt. %, at least 70 wt. %, or at least 90 wt. %; optionally 100 wt. %, less than 100 wt. %, less than 80 wt. %, less than 60 wt. %, or less than 40 wt. % and such values can be combined in any manner to form a range, such as 70-100 wt. %) of the compounds set forth in Table C, based on total mass of the one or more oligosaccharides and total mass of the compounds set forth in Table C.

Aspect F14: The method of any one of aspects F1-F13, or any preceding aspect, wherein the one or more oligosaccharides comprise one or more of the 2D NMR correlations described in Table A for CLX 102.

Aspect F15: The method of any one of aspects F1-F14, or any preceding aspect, wherein the one or more oligosaccharides have an absolute viscosity of between about 0.6 and 1.2 mPa*s (e.g., 0.6-0.8, 0.6-1, 0.8-1, 1-2, or 0.8-1.2 mPa*s) at a concentration of 10 mg/ml in water at a temperature of 25° C.

Aspect F16: The method of any one of aspects F1-F15, or any preceding aspect, wherein the one or more oligosaccharides have an absolute viscosity of between about 0.7 and 3 mPa*s (e.g., 0.7-1.5, 0.7-2.5, 1.5-2.5, or 1-3 mPa*s) at a concentration of 50 mg/ml in water at a temperature of 25° C.

Aspect F17: The method of any one of aspects F1-F16, or any preceding aspect, wherein the one or more oligosaccharides have an absolute viscosity of between about 0.7 and 8 mPa*s (e.g., 0.7-2, 0.7-4, 0.7-6, 1-3, 1-5, 1-8, 2-4, 2-6, 2-8, 4-6, 4-8, 6-8, 1-4, 4-6, or 6-8) at a concentration of 100 mg/ml in water at a temperature of 25° C.

Aspect F18: The method of any one of aspects F1-F17, or any preceding aspect, wherein the method enhances microbial production of at least one of short chain fatty acids, butyrate, lactate, acetate, 3-hydroxybutyrate, nicotinic acid, gamma aminobutyric acid, glutamate, ornithine, or any combination thereof.

Aspect F19: The method of any one of aspects F1-F18, or any preceding aspect, wherein the method increases abundance of at least one of Lactobacillus crispatus, Bifidobacterium pseudocatenatum, Firmicutes, Clostridium butyricum, Blautia, Roseburia, or any combination thereof.

Aspect F20: The method of any one of aspects F1-F19, or any preceding aspect, wherein the method slows microbial utilization of choline.

Aspect F21: The method of any one of aspects F1-F20, or any preceding aspect, wherein the method decreases microbial production of putrescine.

Aspect F22: The method of any one of aspects F1-F21, or any preceding aspect, wherein the oligosaccharide composition further comprises at least one microorganism.

Aspect F23: The method of aspect F22, or any preceding aspect, wherein the at least one microorganism is modulated by the oligosaccharide composition.

Aspect F24: The method of aspect F22 or F23, or any preceding aspect, wherein the at least one microorganism is at least one of Lactobacillus crispatus, Bifidobacterium pseudocatenulatum, Firmicutes, Clostridium butyricum, Blautia, Roseburia, or any combination thereof.

Aspect G1a: A method for modulating microbiota and/or their metabolic products, the method comprising contacting the microbiota with an oligosaccharide composition, wherein the oligosaccharide composition comprises, or collectively comprises, one or more oligosaccharides with glucose, xylose, and galactose subunits.

Aspect G1b: A method for prevention or treatment of a cardiovascular disorder which comprises administering to a patient in need thereof an amount, or a therapeutically effective amount, of an oligosaccharide composition, wherein the oligosaccharide composition comprises, or collectively comprises, one or more oligosaccharides with glucose, xylose, and galactose subunits.

Aspect G1c: A method for prevention or treatment of a renal disorder which comprises administering to a patient in need thereof an amount, or a therapeutically effective amount, of an oligosaccharide composition, wherein the oligosaccharide composition comprises, or collectively comprises, one or more oligosaccharides with glucose, xylose, and galactose subunits.

Aspect G1d: A method for prevention or treatment of a nervous system disorder which comprises administering to a patient in need thereof an amount, or a therapeutically effective amount, of an oligosaccharide composition, wherein the oligosaccharide composition comprises, or collectively comprises, one or more oligosaccharides with glucose, xylose, and galactose subunits.

Aspect G1e: A method for prevention or treatment of an immune system disorder which comprises administering to a patient in need thereof an amount, or a therapeutically effective amount, of an oligosaccharide composition, wherein the oligosaccharide composition comprises, or collectively comprises, one or more oligosaccharides with glucose, xylose, and galactose subunits.

Aspect G1f: A method for prevention or treatment of inflammatory bowel disease which comprises administering to a patient in need thereof an amount, or a therapeutically effective amount, of an oligosaccharide composition, wherein the oligosaccharide composition comprises, or collectively comprises, one or more oligosaccharides with glucose, xylose, and galactose subunits.

Aspect G2: The method of aspect Gi (i.e., any one of aspects G1a-G1f), or any preceding aspect, wherein at least 30 wt. % (e.g., at least 50 wt. %, at least 70 wt. %, or at least 90 wt. %; optionally 100 wt. %, less than 100 wt. %, less than 80 wt. %, less than 60 wt. %, or less than 40 wt. % and such values can be combined in any manner to form a range, such as 30-100 wt. %) of the one or more oligosaccharides is between DP3 and DP 30 (e.g., a DP value of 3-4, 3-5, 3-6, 3-8, 3-10, 3-15, 3-20, 3-25, 3-30, 5-10, 5-15, 5-20, 5-25, 5-30, 8-10, 8-15, 8-20, 8-25, 8-30, 10-15, 10-20, 10-25, 10-30, 15-20, 15-25, 15-30, 20-25, 20-30, 25-30, 3-5, 5-8, 8-10, 10-15, 15-20, 20-25, or 25-30), based on total mass of the one or more oligosaccharides, as measured by oligosaccharide analysis.

Aspect G3: The method of aspect G1 or G2, or any preceding aspect, wherein the one or more oligosaccharides comprise at least 10% (e.g., at least 30%, at least 50%, at least 70%, or at least 90%; optionally 100%, less than 100%, less than 80%, less than 60%, less than 40%, or less than 20%, and such values can be combined in any manner to form a range, such as 10-100%) glucose, xylose, and galactose subunits as calculated by hydrolytic monosaccharide compositional analysis.

Aspect G4: The method of any one of aspects G1-G3, or any preceding aspect, wherein the galactose:glucose ratio is between about 0:1 and 1:1 (e.g., 0.1:1 to 0.4:1, 0.1:1 to 1:1, 0.2:1 to 0.5:1, 0.2:1 to 0.8:1, or 0.2:1 to 0.6:1), or about 0.3:1, and the xylose:glucose ratio is between about 0.1:1 and 1:1 (e.g., 0.1:1 to 0.4:1, 0.1:1 to 1:1, 0.2:1 to 0.5:1, 0.2:1 to 0.8:1, or 0.2:1 to 0.6:1), as calculated by hydrolytic monosaccharide compositional analysis.

Aspect G5: The method of any one of aspects G1-G4, or any preceding aspect, wherein the galactose:glucose ratio is about 0.3:1 and the xylose:glucose ratio is about 0.75:1, as calculated by hydrolytic monosaccharide compositional analysis.

Aspect G6: The method of any one of aspects G1-G5, or any preceding aspect, wherein the glucose subunits comprise 4-linkages, 6-linkages, branched 4,6-linkages, and terminal linkages in a total amount of at least 10% (e.g., at least 30%, at least 50%, at least 70%, or at least 90%; optionally 100%, less than 100%, less than 80%, less than 60%, less than 40%, or less than 20%, and such values can be combined in any manner to form a range, such as 10-100%), as determined by glycosidic linkage composition analysis.

Aspect G7: The method of any one of aspects G1-G6, or any preceding aspect, wherein the glucose subunits comprise 4-linkages, 6-linkages, branched 4,6-linkages, and terminal linkages in a ratio of about 6-7.5:4-6:0.5-2.5:1, or about 6.7:4.8:1.3:1., as determined by glycosidic linkage composition analysis.

Aspect G8: The method of any one of aspects G1-G7, or any preceding aspect, wherein the xylose subunits comprise 2-linkages and terminal linkages in a ratio of about 0.3-0.8:1, or about 0.54:1, as determined by glycosidic linkage composition analysis.

Aspect G9: The method of any one of aspects G1-G8, or any preceding aspect, wherein the xylose linkages subunits comprise 2-linkages and terminal linkages in a total amount of at least 10% (e.g., at least 30%, at least 50%, at least 70%, or at least 90%; optionally 100%, less than 100%, less than 80%, less than 60%, less than 40%, or less than 20%, and such values can be combined in any manner to form a range, such as 10-100%), as determined by glycosidic linkage composition analysis.

Aspect G10: The method of any one of aspects G1-G9, or any preceding aspect, wherein the galactose subunits comprise terminal linkages in an amount of at least 10% (e.g., at least 30%, at least 50%, at least 70%, or at least 90%; optionally 100%, less than 100%, less than 80%, less than 60%, less than 40%, or less than 20%, and such values can be combined in any manner to form a range, such as 10-100%), as determined by glycosidic linkage composition analysis.

Aspect G11: The method of any one of aspects GI-G10, or any preceding aspect, wherein the ratio of terminal glucose:xylose is between 0.1:1 and 1:1 (e.g., 0.1:1 to 0.4:1, 0.1:1 to 1:1, 0.2:1 to 0.5:1, 0.2:1 to 0.8:1, or 0.2:1 to 0.6:1) and the ratio of terminal glucose:galactose is between 0.1:1 and 1:1 (e.g., 0.1:1 to 0.4:1, 0.1:1 to 1:1, 0.2:1 to 0.5:1, 0.2:1 to 0.8:1, or 0.2:1 to 0.6:1), as calculated by hydrolytic monosaccharide compositional analysis.

Aspect G12: The method of any one of aspects GI-GI 1, or any preceding aspect, wherein the ratio of terminal glucose:xylose:galactose is about 0.3:0.5:1 (e.g., 0.2-0.4:0.4-0.6:1), as calculated by hydrolytic monosaccharide compositional analysis.

Aspect G13: The method of any one of aspects G1-G12, or any preceding aspect, wherein a backbone of the one or more oligosaccharides comprises glucose that is beta 4-linked, as determined by glycosidic linkage composition analysis.

Aspect G14: The method of any one of aspects G6-G13, or any preceding aspect, wherein the glucose 6-linkages are alpha branched to xylose subunits, as determined by glycosidic linkage composition analysis.

Aspect G15: The method of any one of aspects G8-G14, or any preceding aspect, wherein the xylose 2-linkages are beta linked to galactose subunits, as determined by glycosidic linkage composition analysis.

Aspect G16: The method of any one of aspects G1-G15, or any preceding aspect, wherein the one or more oligosaccharides comprise one or more of the compounds set forth in Table K.

Aspect G17: The method of any one of aspects G1-G16, or any preceding aspect, wherein the one or more oligosaccharides comprise at least 30 wt. % (e.g., at least 50 wt. %, at least 70 wt. %, or at least 90 wt. %; optionally 100 wt. %, less than 100 wt. %, less than 80 wt. %, less than 60 wt. %, or less than 40 wt. %, and such values can be combined in any manner to form a range, such as 30-100 wt. %) of the compounds set forth in Table K, based on total mass of the one or more oligosaccharides and total mass of the compounds set forth in Table K.

Aspect G18: The method of any one of aspects G1-G17, or any preceding aspect, wherein the one or more oligosaccharides comprise one or more of the 2D NMR correlations described in Table A for CLX 113.

Aspect G19: The method of any one of aspects G1-G18, or any preceding aspect, wherein the one or more oligosaccharides have an absolute viscosity of between about 0.6 and 1.2 mPa*s (e.g., 0.6-0.8, 0.6-1, 0.8-1, 1-2, or 0.8-1.2 mPa*s) at a concentration of 10 mg/ml in water at a temperature of 25° C.

Aspect G20: The method of any one of aspects G1-G19, or any preceding aspect, wherein the one or more oligosaccharides have an absolute viscosity of between about 0.7 and 3 mPa*s (e.g., 0.7-1.5, 0.7-2.5, 1.5-2.5, or 1-3 mPa*s) at a concentration of 50 mg/ml in water at a temperature of 25° C.

Aspect G21: The method of any one of aspects G1-G20, or any preceding aspect, wherein the one or more oligosaccharides have an absolute viscosity of between about 0.7 and 8 mPa*s (e.g., 0.7-2, 0.7-4, 0.7-6, 1-3, 1-5, 1-8, 2-4, 2-6, 2-8, 4-6, 4-8, 6-8, 1-4, 4-6, or 6-8) at a concentration of 100 mg/ml in water at a temperature of 25° C.

Aspect G22: The method of any one of aspects G1-G21, or any preceding aspect, wherein the method enhances microbial production of short chain fatty acids, butyrate, acetate, succinate, pantothenic acid, isoleucine, gamma aminobutyric acid, ornithine, or any combination thereof.

Aspect G23: The method of any one of aspects G1-G22, or any preceding aspect, wherein the method increases abundance of at least one of Bifidobacteria, Bifidobacterium animalis, Firmicutes, Clostridium butyricum, Ruminococcus, Ruminococcus gnavus, Ruminococcus torques, Blautia, Roseburia, Faecalibacterium, or any combination thereof.

Aspect G24: The method of any one of aspects G1-G23, or any preceding aspect, wherein the method decreases microbial production of at least one of histamine, cadaverine, putrescine, or a combination thereof.

Aspect G25: The method of any one of aspects G1-G24, or any preceding aspect, wherein the oligosaccharide composition further comprises at least one microorganism.

Aspect G26: The method of aspect G25, or any preceding aspect, wherein the at least one microorganism is modulated by the oligosaccharide composition.

Aspect G27: The method of aspect G25 or G26, or any preceding aspect, wherein the at least one microorganism is at least one of Bifidobacteria, Bifidobacterium animalis, Firmicutes, Clostridium butyricum, Ruminococcus, Ruminococcus gnavus, Ruminococcus torques, Blautia, Roseburia, Faecalibacterium, or any combination thereof.

Aspect H1a: A method for modulating microbiota and/or their metabolic products, the method comprising contacting the microbiota with an oligosaccharide composition, wherein the oligosaccharide composition comprises, or collectively comprises, one or more oligosaccharides with galactose and mannose subunits.

Aspect H1b: A method for prevention or treatment of a cardiovascular disorder which comprises administering to a patient in need thereof an amount, or a therapeutically effective amount, of an oligosaccharide composition, wherein the oligosaccharide composition comprises, or collectively comprises, one or more oligosaccharides with galactose and mannose subunits.

Aspect H1c: A method for prevention or treatment of a renal disorder which comprises administering to a patient in need thereof an amount, or a therapeutically effective amount, of an oligosaccharide composition, wherein the oligosaccharide composition comprises, or collectively comprises, one or more oligosaccharides with galactose and mannose subunits.

Aspect H1d: A method for prevention or treatment of a nervous system disorder which comprises administering to a patient in need thereof an amount, or a therapeutically effective amount, of an oligosaccharide composition, wherein the oligosaccharide composition comprises, or collectively comprises, one or more oligosaccharides with galactose and mannose subunits.

Aspect H1e: A method for prevention or treatment of an immune system disorder which comprises administering to a patient in need thereof an amount, or a therapeutically effective amount, of an oligosaccharide composition, wherein the oligosaccharide composition comprises, or collectively comprises, one or more oligosaccharides with galactose and mannose subunits.

Aspect H1f: A method for prevention or treatment of inflammatory bowel disease which comprises administering to a patient in need thereof an amount, or a therapeutically effective amount, of an oligosaccharide composition, wherein the oligosaccharide composition comprises, or collectively comprises, one or more oligosaccharides with galactose and mannose subunits.

Aspect H2: The method of aspect H1 (i.e., any one of aspects H1a-H1f), or any preceding aspect, wherein at least 30 wt. % (e.g., at least 50 wt. %, at least 70 wt. %, or at least 90 wt. %; optionally 100 wt. %, less than 100 wt. %, less than 80 wt. %, less than 60 wt. %, or less than 40 wt. % and such values can be combined in any manner to form a range, such as 50 wt. % to less than 100%) of the one or more oligosaccharides is between DP3 and DP 30 (e.g., a DP value of 3-4, 3-5, 3-6, 3-8, 3-10, 3-15, 3-20, 3-25, 3-30, 5-10, 5-15, 5-20, 5-25, 5-30, 8-10, 8-15, 8-20, 8-25, 8-30, 10-15, 10-20, 10-25, 10-30, 15-20, 15-25, 15-30, 20-25, 20-30, 25-30, 3-5, 5-8, 8-10, 10-15, 15-20, 20-25, or 25-30), based on total mass of the one or more oligosaccharides, as measured by oligosaccharide analysis.

Aspect H3: The method of aspect H1 or H2, or any preceding aspect, wherein the one or more oligosaccharides comprise at least 10% (e.g., at least 30%, at least 50%, at least 70%, or at least 90%; optionally 100%, less than 100%, less than 80%, less than 60%, less than 40%, or less than 20%, and such values can be combined in any manner to form a range, such as 30-80%) galactose and mannose subunits, as calculated by hydrolytic monosaccharide compositional analysis.

Aspect H4: The method of any one of aspects H1-H3, or any preceding aspect, wherein the galactose:mannose ratio is between about 0.1:1 and 1:1 (e.g., 0.1:1 to 0.4:1, 0.1:1 to 1:1, 0.2:1 to 0.5:1, 0.2:1 to 0.8:1, or 0.2:1 to 0.6:1), as calculated by hydrolytic monosaccharide compositional analysis.

Aspect H5: The method of any one of aspects H1-H4, or any preceding aspect, wherein the galactose:mannose ratio is about 0.25:1 (e.g., 0.1:1 to 0.3:1).

Aspect H6: The method of any one of aspects H1-H5, or any preceding aspect, wherein the mannose subunits comprise 4-linkages, branched 4-6 linkages, and terminal linkages in a total amount of at least 10% (e.g., at least 30%, at least 50%, at least 70%, or at least 90%; optionally 100%, less than 100%, less than 80%, less than 60%, less than 40%, or less than 20%, and such values can be combined in any manner to form a range, such as 10-100%), as determined by glycosidic linkage composition analysis.

Aspect H7: The method of any one of aspects H1-H6, or any preceding aspect, wherein the mannose subunits comprise 4-linkages, branched 4-6 linkages, and terminal linkages in a ratio of about 1.5-3.5:0.2-0.5:1, or about 2.3:0.31:1, as determined by glycosidic linkage composition analysis.

Aspect H8: The method of any one of aspects H1-H7, or any preceding aspect, wherein the galactose subunits comprise terminal linkages in an amount of at least 10% (e.g., at least 30%, at least 50%, at least 70%, or at least 90%; optionally 100%, less than 100%, less than 80%, less than 60%, less than 40%, or less than 20%, and such values can be combined in any manner to form a range, such as 10-100%), as determined by glycosidic linkage composition analysis.

Aspect H9: The method of any one of aspects H1-H8, or any preceding aspect, wherein a backbone of the one or more oligosaccharides comprises mannose that is beta 4-linked, as determined by glycosidic linkage composition analysis.

Aspect H10: The method of any one of aspects H6-H9, or any preceding aspect, wherein the mannose 6-linkages are alpha branched to galactose subunits, as determined by glycosidic linkage composition analysis.

Aspect H11: The method of any one of aspects H1-H10, or any preceding aspect, wherein the one or more oligosaccharides comprise one or more of the compounds set forth in Table F.

Aspect H12: The method of any one of aspects H1-H11, or any preceding aspect, wherein the one or more oligosaccharides comprise at least 30 wt. % (e.g., at least 50 wt. %, at least 70 wt. %, or at least 90 wt. %; optionally 100 wt. %, less than 100 wt. %, less than 80 wt. %, less than 60 wt. %, or less than 40 wt. % and such values can be combined in any manner to form a range, such as 70-80 wt. %) of the compounds set forth in Table F, based on total mass of the one or more oligosaccharides and total mass of the compounds set forth in Table F.

Aspect H13: The method of any one of aspects H1-H12, or any preceding aspect, wherein the one or more oligosaccharides comprise one or more of the 2D NMR correlations described in Table A for CLX 108.

Aspect H14: The method of any one of aspects H1-H13, or any preceding aspect, wherein the one or more oligosaccharides have an absolute viscosity of between about 0.6 and 1.2 mPa*s (e.g., 0.6-0.8, 0.6-1, 0.8-1, 1-2, or 0.8-1.2 mPa*s) at a concentration of 10 mg/ml in water at a temperature of 25° C.

Aspect H15: The method of any one of aspects H1-H14, or any preceding aspect, wherein the one or more oligosaccharides have an absolute viscosity of between about 0.7 and 3 mPa*s (e.g., 0.7-1.5, 0.7-2.5, 1.5-2.5, or 1-3 mPa*s) at a concentration of 50 mg/ml in water at a temperature of 25° C.

Aspect H16: The method of any one of aspects H1-H15, or any preceding aspect, wherein the one or more oligosaccharides have an absolute viscosity of between about 0.7 and 8 mPa*s (e.g., 0.7-2, 0.7-4, 0.7-6, 1-3, 1-5, 1-8, 2-4, 2-6, 2-8, 4-6, 4-8, 6-8, 1-4, 4-6, or 6-8) at a concentration of 100 mg/ml in water at a temperature of 25° C.

Aspect H17: The method of any one of aspects H1-H16, or any preceding aspect, wherein the one or more oligosaccharides comprise one or more of the compounds set forth in Table I.

Aspect H18: The method of any one of aspects H1-H17, or any preceding aspect, wherein the one or more oligosaccharides comprise at least 30 wt. % (e.g., at least 50 wt. %, at least 70 wt. %, or at least 90 wt. %; optionally 100 wt. %, less than 100 wt. %, less than 80 wt. %, less than 60 wt. %, or less than 40 wt. % and such values can be combined in any manner to form a range, such as 30-60%) of the compounds set forth in Table I, based on total mass of the one or more oligosaccharides and total mass of the compounds set forth in Table I.

Aspect H19: The method of any one of aspects H1-H18, or any preceding aspect, wherein the one or more oligosaccharides comprise one or more of the 2D NMR correlations described in Table A for CLX 111.

Aspect H20: The method of any one of aspects H1-H19, or any preceding aspect, wherein the method enhances microbial production of at least one of short chain fatty acids, butyrate, propionate, 3-hydroxybutyrate, pantothenic acid, isoleucine, gamma aminobutyric acid, glutamate, ornithine, or any combination thereof.

Aspect H21: The method of any one of aspects H1-H20, or any preceding aspect, wherein the method increases abundance of at least one of Bifidobacteria, Bifidobacterium pseudocatenulatum, Bifidobacterium animalis, Firmicutes, Clostridium butyricum, Ruminococcus, Ruminococcus gnavus, Blautia, Faecalibacterium, or any combination thereof.

Aspect H22: The method of any one of aspects H1-H21, or any preceding aspect, wherein the method slows microbial utilization of choline.

Aspect H23: The method of any one of aspects H1-H22, or any preceding aspect, wherein the method decreases microbial utilization of methionine.

Aspect H24: The method of any one of aspects H1-H23, or any preceding aspect, wherein the method decreases microbial production of histamine, putrescine, or both.

Aspect H25: The method of any one of aspects H1-H24, or any preceding aspect, wherein the oligosaccharide composition further comprises at least one microorganism.

Aspect H26 The method of aspect H25, or any preceding aspect, wherein the at least one microorganism is modulated by the oligosaccharide composition.

Aspect H27: The method of aspect1 H25 or H26, or any preceding aspect, wherein the at least one microorganism is at least one of Bifidobacteria, Bifidobacterium pseudocatenulatum, Bifidobacterium animalis, Firmicutes, Clostridium butyricum, Ruminococcus, Ruminococcus gnavus, Blautia, Faecalibacterium, or any combination thereof.

Aspect I1a: A method for modulating microbiota and/or their metabolic products, the method comprising contacting the microbiota with an oligosaccharide composition, wherein the oligosaccharide composition comprises, or collectively comprises, one or more oligosaccharides with arabinose and xylose subunits.

Aspect I1b: A method for prevention or treatment of a cardiovascular disorder which comprises administering to a patient in need thereof an amount, or a therapeutically effective amount, of an oligosaccharide composition, wherein the oligosaccharide composition comprises, or collectively comprises, one or more oligosaccharides with arabinose and xylose subunits.

Aspect I1c: A method for prevention or treatment of a renal disorder which comprises administering to a patient in need thereof an amount, or a therapeutically effective amount, of an oligosaccharide composition, wherein the oligosaccharide composition comprises, or collectively comprises, one or more oligosaccharides with arabinose and xylose subunits.

Aspect I1d: A method for prevention or treatment of a nervous system disorder which comprises administering to a patient in need thereof an amount, or a therapeutically effective amount, of an oligosaccharide composition, wherein the oligosaccharide composition comprises, or collectively comprises, one or more oligosaccharides with arabinose and xylose subunits.

Aspect I1e: A method for prevention or treatment of an immune system disorder which comprises administering to a patient in need thereof an amount, or a therapeutically effective amount, of an oligosaccharide composition, wherein the oligosaccharide composition comprises, or collectively comprises, one or more oligosaccharides with arabinose and xylose subunits.

Aspect I1f: A method for prevention or treatment of inflammatory bowel disease which comprises administering to a patient in need thereof an amount, or a therapeutically effective amount, of an oligosaccharide composition, wherein the oligosaccharide composition comprises, or collectively comprises, one or more oligosaccharides with arabinose and xylose subunits.

Aspect I2: The method of aspect I1 (i.e., any one of aspects I1a-I1f), or any preceding aspect, wherein at least 30 wt. % (e.g., at least 50 wt. %, at least 70 wt. %, or at least 90 wt. %; optionally 100 wt. %, less than 100 wt. %, less than 80 wt. %, less than 60 wt. %, or less than 40 wt. % and such values can be combined in any manner to form a range, such as 30-60 wt. %) of one or more oligosaccharides is between DP3 and DP 30 (e.g., a DP value of 3-4, 3-5, 3-6, 3-8, 3-10, 3-15, 3-20, 3-25, 3-30, 5-10, 5-15, 5-20, 5-25, 5-30, 8-10, 8-15, 8-20, 8-25, 8-30, 10-15, 10-20, 10-25, 10-30, 15-20, 15-25, 15-30, 20-25, 20-30, 25-30, 3-5, 5-8, 8-10, 10-15, 15-20, 20-25, or 25-30), based on total mass of one or more oligosaccharides, as measured by oligosaccharide analysis.

Aspect I3: The method of aspect I1 or 12, or any preceding aspect, wherein the one or more oligosaccharides comprise at least 30% (e.g., at least 50%, at least 70%, or at least 90%; optionally 100%, less than 100%, less than 80%, less than 60%, or less than 40% and such values can be combined in any manner to form a range, such as 30-60%) arabinose and xylose subunits, as calculated by hydrolytic monosaccharide compositional analysis.

Aspect I4: The method of any one of aspects I1-I3, or any preceding aspect, wherein the arabinose:xylose ratio is between about 0.05-2:1 (e.g., 0.05:1 to 1:1, 0.1:1 to 0.8:1, 0.2:1 to 1.2:1, or 0.5:1 to 1.5:1), as determined by glycosidic linkage composition analysis.

Aspect I5: The method of any one of aspects I1-I4, or any preceding aspect, wherein the arabinose:xylose ratio is about 0.6:1, as determined by glycosidic linkage composition analysis.

Aspect I6: The method of any one of aspects I1-I5, or any preceding aspect, wherein the xylose subunits comprise 4-linkages, branched 4,2-linkages, branched 4,3-linkages, trisecting 2,3,4-linkages, and terminal linkages in a total amount of at least 10% (e.g., at least 30%, at least 50%, at least 70%, or at least 90%; optionally 100%, less than 100%, less than 80%, less than 60%, less than 40%, or less than 20%, and such values can be combined in any manner to form a range, such as 10-100%), as determined by glycosidic linkage composition analysis.

Aspect I7: The method of any one of aspects I1-I6, or any preceding aspect, wherein the xylose subunits comprise 4-linkages, branched 4,2- and 4,3-linkages, trisecting 2,3,4-linkages, and terminal linkages in a ratio of about 10-13:0.5-3:7-10:0.5-3:1, or about 11.8:1.4:8.4:1.6:1, as determined by glycosidic linkage composition analysis.

Aspect I8: The method of any one of aspects I1-I7, or any preceding aspect, wherein the arabinose subunits comprise terminal linkages in an amount of at least 10% (e.g., at least 30%, at least 50%, at least 70%, or at least 90%; optionally 100%, less than 100%, less than 80%, less than 60%, less than 40%, or less than 20%, and such values can be combined in any manner to form a range, such as 90% to less than 100%), as determined by glycosidic linkage composition analysis.

Aspect I9: The method of any one of aspects I1-I8, or any preceding aspect, wherein a backbone of the one or more oligosaccharides comprises xylose that is beta 4-linked, as determined by glycosidic linkage composition analysis.

Aspect I10: The method of any one of aspects I6-I9, or any preceding aspect, wherein the xylose 2- and 3-linkages are alpha branched to arabinose subunits, as determined by glycosidic linkage composition analysis.

Aspect I11: The method of any one of aspects I1-I10, or any preceding aspect, wherein the one or more oligosaccharides comprise one or more of the compounds set forth in Table L.

Aspect I12: The method of any one of aspects I1-I11, or any preceding aspect, wherein the one or more oligosaccharides comprise at least 30 wt. % (e.g., at least 50 wt. %, at least 70 wt. %, or at least 90 wt. %; optionally 100 wt. %, less than 100 wt. %, less than 80 wt. %, less than 60 wt. %, or less than 40 wt. % and such values can be combined in any manner to form a range, such as 90-100 wt. %) of the compounds set forth in Table L, based on total mass of the one or more oligosaccharides and total mass of the compounds set forth in Table L.

Aspect I13: The method of any one of aspects I1-I12, or any preceding aspect, wherein the one or more oligosaccharides comprise one or more the 2D NMR correlations described in Table A for CLX 114.

Aspect I14: The method of any one of aspects I1-I13, or any preceding aspect, wherein the one or more oligosaccharides have an absolute viscosity of between about 0.6 and 1.2 mPa*s (e.g., 0.6-0.8, 0.6-1, 0.8-1, 1-2, or 0.8-1.2 mPa*s) at a concentration of 10 mg/ml in water at a temperature of 25° C.

Aspect I15: The method of any one of aspects I1-I14, or any preceding aspect, wherein the one or more oligosaccharides have an absolute viscosity of between about 0.7 and 3 mPa*s (e.g., 0.7-1.5, 0.7-2.5, 1.5-2.5, or 1-3 mPa*s) at a concentration of 50 mg/ml in water at a temperature of 25° C.

Aspect I16: The method of any one of aspects I1-I15, or any preceding aspect, wherein the one or more oligosaccharides have an absolute viscosity of between about 0.7 and 8 mPa*s (e.g., 0.7-2, 0.7-4, 0.7-6, 1-3, 1-5, 1-8, 2-4, 2-6, 2-8, 4-6, 4-8, 6-8, 1-4, 4-6, or 6-8) at a concentration of 100 mg/ml in water at a temperature of 25° C.

Aspect I17: The method of any one of aspects I1-I16, or any preceding aspect, wherein the method enhances microbial production of at least one of short chain fatty acids, butyrate, lactate, acetate, succinate, glycerate, nicotinic acid, pantothenic acid, isoleucine, valine, gamma aminobutyric acid, ornithine, or any combination thereof.

Aspect I18: The method of any one of aspects I1-I17, or any preceding aspect, wherein the method increases abundance of at least one of Bifidobacteria, Bifidobacterium pseudocatenulatum, Bifidobacterium animalis, Bacteroides, Bacteroides ovatus, Ruminococcus, Ruminococcus gnavus, Ruminococcus torques, Blautia, Roseburia, Faecalibacteria, or any combination thereof.

Aspect I19: The method of any one of aspects I1-I18, or any preceding aspect, wherein the method decreases microbial production of at least one of histamine, cadaverine, putrescine, or a combination thereof.

Aspect I20: The method of any one of aspects 11-119, or any preceding aspect, wherein the oligosaccharide composition further comprises at least one microorganism.

Aspect I21: The method of aspect I20, or any preceding aspect, wherein the at least one microorganism is modulated by the oligosaccharide composition.

Aspect I22: The method of aspect I20 or 121, or any preceding aspect, wherein the at least one microorganism is at least one of Bifidobacteria, Bifidobacterium pseudocatenulatum, Bifidobacterium animalis, Bacteroides, Bacteroides ovatus, Ruminococcus, Ruminococcus gnavus, Ruminococcus torques, Blautia, Roseburia, Faecalibacteria, or any combination thereof.

Aspect J1a: A method for modulating microbiota and/or their metabolic products, the method comprising contacting the microbiota with an oligosaccharide composition, wherein the oligosaccharide composition comprises, or collectively comprises, one or more oligosaccharides with arabinose and galactose subunits.

Aspect J1b: A method for prevention or treatment of a cardiovascular disorder which comprises administering to a patient in need thereof an amount, or a therapeutically effective amount, of an oligosaccharide composition, wherein the oligosaccharide composition comprises, or collectively comprises, one or more oligosaccharides with arabinose and galactose subunits.

Aspect J1c: A method for prevention or treatment of a renal disorder which comprises administering to a patient in need thereof an amount, or a therapeutically effective amount, of an oligosaccharide composition, wherein the oligosaccharide composition comprises, or collectively comprises, one or more oligosaccharides with arabinose and galactose subunits.

Aspect J1d: A method for prevention or treatment of a nervous system disorder which comprises administering to a patient in need thereof an amount, or a therapeutically effective amount, of an oligosaccharide composition, wherein the oligosaccharide composition comprises, or collectively comprises, one or more oligosaccharides with arabinose and galactose subunits.

Aspect J1e: A method for prevention or treatment of an immune system disorder which comprises administering to a patient in need thereof an amount, or a therapeutically effective amount, of an oligosaccharide composition, wherein the oligosaccharide composition comprises, or collectively comprises, one or more oligosaccharides with arabinose and galactose subunits.

Aspect J1f: A method for prevention or treatment of inflammatory bowel disease which comprises administering to a patient in need thereof an amount, or a therapeutically effective amount, of an oligosaccharide composition, wherein the oligosaccharide composition comprises, or collectively comprises, one or more oligosaccharides with arabinose and galactose subunits.

Aspect J2: The method of aspect J1 (i.e., any one of aspects J1a-J1f), or any preceding aspect, wherein at least 30 wt. % (e.g., at least 50 wt. %, at least 70 wt. %, or at least 90 wt. %; optionally 100 wt. %, less than 100 wt. %, less than 80 wt. %, less than 60 wt. %, or less than 40 wt. % and such values can be combined in any manner to form a range, such as 70-90 wt. %) of one or more oligosaccharides is between DP3 and DP 30 (e.g., a DP value of 3-4, 3-5, 3-6, 3-8, 3-10, 3-15, 3-20, 3-25, 3-30, 5-10, 5-15, 5-20, 5-25, 5-30, 8-10, 8-15, 8-20, 8-25, 8-30, 10-15, 10-20, 10-25, 10-30, 15-20, 15-25, 15-30, 20-25, 20-30, 25-30, 3-5, 5-8, 8-10, 10-15, 15-20, 20-25, or 25-30), based on total mass of one or more oligosaccharides, as measured by oligosaccharide analysis.

Aspect J3: The method of aspect J1 or J2, any preceding aspect, wherein the one or more oligosaccharides comprise at least 30% (e.g., at least 50%, at least 70%, or at least 90%; optionally 100%, less than 100%, less than 80%, less than 60%, or less than 40% and such values can be combined in any manner to form a range, such as 30-60%) galactose and arabinose subunits, as calculated by hydrolytic monosaccharide compositional analysis.

Aspect J4: The method of any one of aspects J1-J3, any preceding aspect, wherein the arabinose:galactose ratio is between about 0.01:1 and 1:1 (e.g., 0.01:1 to 1:1, 0.02:1 to 0.15:1, 0.05:1 to 0.1:1, 0.04:1 to 0.2:1, or 0.4:1 to 0.8:1).

Aspect J5: The method of any one of aspects J1-J4, any preceding aspect, wherein the arabinose:galactose ratio is about 0.08:1.

Aspect J6: The method of any one of aspects J1-J5, any preceding aspect, wherein the galactose subunits comprise 3-linkages, 6-linkages, and branched 3,6-linkages in a total amount of at least 10% (e.g., at least 30%, at least 50%, at least 70%, or at least 90%; optionally 100%, less than 100%, less than 80%, less than 60%, less than 40%, or less than 20%, and such values can be combined in any manner to form a range, such as 30-80%), as calculated by hydrolytic monosaccharide compositional analysis.

Aspect J7: The method of any one of aspects J1-J6, any preceding aspect, wherein the galactose subunits comprise 3-linkages, 6-linkages, branched 3,6-linkages, and terminal linkages in a ratio of about 0.1-0.6:0.1-0.5:0.1-0.6:1, or about 0.3:0.2:0.3:1, as determined by glycosidic linkage composition analysis.

Aspect J8: The method of any one of aspects J1-J7, any preceding aspect, wherein the arabinose subunits comprise terminal linkages in an amount of at least 10% (e.g., at least 30%, at least 50%, at least 70%, or at least 90%; optionally 100%, less than 100%, less than 80%, less than 60%, less than 40%, or less than 20%, and such values can be combined in any manner to form a range, such as 10-100%), as determined by glycosidic linkage composition analysis.

Aspect J9: The method of any one of aspects J1-J8, any preceding aspect, wherein a backbone of the one or more oligosaccharides comprises galactose that is beta 3-linked, as determined by glycosidic linkage composition analysis.

Aspect J10: The method of any one of aspects J6-J9, any preceding aspect, wherein the galactose 3,6-linkages are alpha branched to arabinose subunits or beta branched to galactose subunits that are extended in a 6-linked beta fashion.

Aspect J11: The method of any one of aspects J1-J10, any preceding aspect, wherein the one or more oligosaccharides comprise one or more of the compounds set forth in Table E.

Aspect J12: The method of any one of aspects J1-J11, any preceding aspect, wherein the one or more oligosaccharides comprise at least 0% (e.g., at least 50%, at least 70%, or at least 90%; optionally 100%, less than 100%, less than 80%, less than 60%, or less than 40%, and such values can be combined in any manner to form a range, such as 30-100%) of the compounds set forth in Table E, based on total mass of the one or more oligosaccharides and total mass of the compounds set forth in Table E.

Aspect J13: The method of any one of aspects J1-J12, any preceding aspect, wherein the one or more oligosaccharides comprise one or more of the 2D NMR correlations described in Table A for CLX 105.

Aspect J14: The method of any one of aspects J1-J13, any preceding aspect, wherein the one or more oligosaccharides have an absolute viscosity of between about 0.6 and 1.2 mPa*s (e.g., 0.6-0.8, 0.6-1, 0.8-1, 1-2, or 0.8-1.2 mPa*s) at a concentration of 10 mg/ml in water at a temperature of 25° C.

Aspect J15: The method of any one of aspects J1-J14, any preceding aspect, wherein the one or more oligosaccharides have an absolute viscosity of between about 0.7 and 3 mPa*s (e.g., 0.7-1.5, 0.7-2.5, 1.5-2.5, or 1-3 mPa*s) at a concentration of 50 mg/ml in water at a temperature of 25° C.

Aspect J16: The method of any one of aspects J1-J15, any preceding aspect, wherein the one or more oligosaccharides have an absolute viscosity of between about 0.7 and 8 mPa*s (e.g., 0.7-2, 0.7-4, 0.7-6, 1-3, 1-5, 1-8, 2-4, 2-6, 2-8, 4-6, 4-8, 6-8, 1-4, 4-6, or 6-8) at a concentration of 100 mg/ml in water at a temperature of 25° C.

Aspect J17: The method of any one of aspects J1-J16, any preceding aspect, wherein the method enhances microbial production of at least one of short chain fatty acids, butyrate, propionate, acetate, pantothenic acid, isoleucine, gamma aminobutyric acid, or any combination thereof.

Aspect J18: The method of any one of aspects J1-J17, any preceding aspect, wherein the method increases abundance of at least one of Bifidobacterium Longum ssp Infantis, Bifidobacterium pseudocatenulatum, Bifidobacterium Longum ssp longum, Bifidobacteria, Bifidobacterium animalis, Bacteroides, Bacteroides ovatus, Clostridium butyricum, Ruminococcus torques, Blautia, Roseburia, Faecalibacterium, or any combination thereof.

Aspect J19: The method of any one of aspects J1-J18, any preceding aspect, wherein the method slows microbial utilization of choline.

Aspect J20: The method of any one of aspects J1-J19, any preceding aspect, wherein the method decreases microbial production of histamine, putrescine, or a combination thereof.

Aspect J21: The method of any one of aspects J1-J20, any preceding aspect, wherein the oligosaccharide composition further comprises at least one microorganism.

Aspect J22: The method of aspect J21, any preceding aspect, wherein the at least one microorganism is modulated by the oligosaccharide composition.

Aspect J23: The method of aspect J21 or J22, any preceding aspect, wherein the at least one microorganism is at least one of Bifidobacterium Longum ssp Infantis, Bifidobacterium pseudocatenulatum, Bifidobacterium Longum ssp longum, Bifidobacteria, Bifidobacterium animalis, Bacteroides, Bacteroides ovatus, Clostridium butyricum, Ruminococcus torques, Blautia, Roseburia, Faecalibacterium, or any combination thereof.

Aspect K1a: A method for modulating microbiota and/or their metabolic products, the method comprising contacting the microbiota with an oligosaccharide composition, wherein the oligosaccharide composition comprises, or collectively comprises, one or more oligosaccharides with galactose, arabinose, rhamnose, and galacturonic acid subunits.

Aspect K1b: A method for prevention or treatment of a cardiovascular disorder which comprises administering to a patient in need thereof an amount, or a therapeutically effective amount, of an oligosaccharide composition, wherein the oligosaccharide composition comprises, or collectively comprises, one or more oligosaccharides with galactose, arabinose, rhamnose, and galacturonic acid subunits.

Aspect K1c: A method for prevention or treatment of a renal disorder which comprises administering to a patient in need thereof an amount, or a therapeutically effective amount, of an oligosaccharide composition, wherein the oligosaccharide composition comprises, or collectively comprises, one or more oligosaccharides with galactose, arabinose, rhamnose, and galacturonic acid subunits.

Aspect K1d: A method for prevention or treatment of a nervous system disorder which comprises administering to a patient in need thereof an amount, or a therapeutically effective amount, of an oligosaccharide composition, wherein the oligosaccharide composition comprises, or collectively comprises, one or more oligosaccharides with galactose, arabinose, rhamnose, and galacturonic acid subunits.

Aspect K1e: A method for prevention or treatment of an immune system disorder which comprises administering to a patient in need thereof an amount, or a therapeutically effective amount, of an oligosaccharide composition, wherein the oligosaccharide composition comprises, or collectively comprises, one or more oligosaccharides with galactose, arabinose, rhamnose, and galacturonic acid subunits.

Aspect K1f: A method for prevention or treatment of inflammatory bowel disease which comprises administering to a patient in need thereof an amount, or a therapeutically effective amount, of an oligosaccharide composition, wherein the oligosaccharide composition comprises, or collectively comprises, one or more oligosaccharides with galactose, arabinose, rhamnose, and galacturonic acid xylose subunits.

Aspect K2: The method of aspect K1 (i.e., any one of aspects K1a-K1f), or any preceding aspect, wherein at least 30 wt. % (e.g., at least 50 wt. %, at least 70 wt. %, or at least 90 wt. %; optionally 100 wt. %, less than 100 wt. %, less than 80 wt. %, less than 60 wt. %, or less than 40 wt. % and such values can be combined in any manner to form a range, such as 30-60 wt. %) of one or more oligosaccharides is between DP3 and DP 30 (e.g., a DP value of 3-4, 3-5, 3-6, 3-8, 3-10, 3-15, 3-20, 3-25, 3-30, 5-10, 5-15, 5-20, 5-25, 5-30, 8-10, 8-15, 8-20, 8-25, 8-30, 10-15, 10-20, 10-25, 10-30, 15-20, 15-25, 15-30, 20-25, 20-30, 25-30, 3-5, 5-8, 8-10, 10-15, 15-20, 20-25, or 25-30), based on total mass of one or more oligosaccharides, as measured by oligosaccharide analysis.

Aspect K3: The method of aspect K1 or K2, or any preceding aspect, wherein the one or more oligosaccharides comprise at least 10% (e.g., at least 30%, at least 50%, at least 70%, or at least 90%; optionally 100%, less than 100%, less than 80%, less than 60%, less than 40%, or less than 20%, and such values can be combined in any manner to form a range, such as 10-100%) galactose, arabinose, rhamnose, and galacturonic acid subunits, as calculated by hydrolytic monosaccharide compositional analysis.

Aspect K4: The method of any one of aspects K1-K3, or any preceding aspect, wherein the galacturonic acid:galactose ratio is between 0.01:1 and 1:1 (e.g., 0.01:1 to 1:1, 0.02:1 to 0.15:1, 0.05:1 to 0.1:1, 0.04:1 to 0.2:1, or 0.4:1 to 0.8:1), the rhamnose:galctose ratio is between 0.01:1 and 1:1 (e.g., 0.01:1 to 1:1, 0.02:1 to 0.15:1, 0.05:1 to 0.1:1, 0.04:1 to 0.2:1, or 0.4:1 to 0.8:1), and the arabinose:galactose ratio is between 0.01:1 and 1:1 (e.g., 0.01:1 to 1:1, 0.02:1 to 0.15:1, 0.05:1 to 0.1:1, 0.04:1 to 0.2:1, or 0.4:1 to 0.8:1), as calculated by hydrolytic monosaccharide compositional analysis.

Aspect K5: The method of any one of aspects K1-K4, or any preceding aspect, wherein the galacturonic acid:galactose ratio is about 0.04:1 (e.g., 0.02:1 to 0.1:1), the rhamnose:galctose ratio is about 0.06:1 (e.g., 0.04:1 to 0.08:1), and the arabinose:galactose ratio is about 0.11:1 (e.g., 0.09:1 to 0.13:1), as calculated by hydrolytic monosaccharide compositional analysis.

Aspect K6: The method of any one of aspects K1-K5, or any preceding aspect, wherein the galactose subunits comprise 4-linkages in an amount of at least 10% (e.g., at least 30%, at least 50%, at least 70%, or at least 90%; optionally 100%, less than 100%, less than 80%, less than 60%, less than 40%, or less than 20%, and such values can be combined in any manner to form a range, such as 10-100%), as determined by glycosidic linkage composition analysis.

Aspect K7: The method of any one of aspects K1-K6, or any preceding aspect, wherein the galactose subunits comprise 4-linkages and terminal linkages in a ratio of about 1-3:1, or about 1.8:1, as determined by glycosidic linkage composition analysis.

Aspect K8: The method of aspect K6 or K7, or any preceding aspect, wherein the 4-galctose linkages are beta linked, as determined by glycosidic linkage composition analysis.

Aspect K9: The method of any one of aspects K1-K8, or any preceding aspect, wherein the arabinose subunits comprise terminal linkages in an amount of at least 10% (e.g., at least 30%, at least 50%, at least 70%, or at least 90%; optionally 100%, less than 100%, less than 80%, less than 60%, less than 40%, or less than 20%, and such values can be combined in any manner to form a range, such as 70-80%), as determined by glycosidic linkage composition analysis.

Aspect K10: The method of any one of aspects K1-K9, or any preceding aspect, wherein the one or more oligosaccharides comprise one or more of the compounds set forth in Table G.

Aspect K11: The method of any one of aspects K1-K10, or any preceding aspect, wherein the one or more oligosaccharides comprise at least 30 wt. % (e.g., at least 50 wt. %, at least 70 wt. %, or at least 90 wt. %; optionally 100 wt. %, less than 100 wt. %, less than 80 wt. %, less than 60 wt. %, or less than 40 wt. %, and such values can be combined in any manner to form a range, such as 30-100 wt. %) of the compounds set forth in Table G, based on total mass of the one or more oligosaccharides and total mass of the compounds set forth in Table G.

Aspect K12: The method of any one of aspects K1-K11, or any preceding aspect, wherein the one or more oligosaccharides comprise one or more of the 2D NMR correlations described in Table A for CLX 109.

Aspect K13: The method of any one of aspects K1-K12, or any preceding aspect, wherein the one or more oligosaccharides have an absolute viscosity of between about 0.6 and 1.2 mPa*s (e.g., 0.6-0.8, 0.6-1, 0.8-1, 1-2, or 0.8-1.2 mPa*s) at a concentration of 10 mg/ml in water at a temperature of 25° C.

Aspect K14: The method of any one of aspects K1-K13, or any preceding aspect, wherein the one or more oligosaccharides have an absolute viscosity of between about 0.7 and 3 mPa*s (e.g., 0.7-1.5, 0.7-2.5, 1.5-2.5, or 1-3 mPa*s) at a concentration of 50 mg/ml in water at a temperature of 25° C.

Aspect K15: The method of any one of aspects K1-K14, or any preceding aspect, wherein the one or more oligosaccharides have an absolute viscosity of between about 0.7 and 8 mPa*s (e.g., 0.7-2, 0.7-4, 0.7-6, 1-3, 1-5, 1-8, 2-4, 2-6, 2-8, 4-6, 4-8, 6-8, 1-4, 4-6, or 6-8) at a concentration of 100 mg/ml in water at a temperature of 25° C.

Aspect K16: The method of any one of aspects K1-K15, or any preceding aspect, wherein the method enhances microbial production of at least one of short chain fatty acids, butyrate, propionate, acetate, pantothenic acid, isoleucine, gamma aminobutyric acid, ornithine, or any combination thereof.

Aspect K17: The method of any one of aspects K1-K16, or any preceding aspect, wherein the method increases abundance of at least one of Lactobacillus crispatus, Lactobacillus Rhamnosus GG, Bifidobacterium Longum ssp Infantis, Bifidobacterium Pseudocatenatum, Bifidobacterium Longum ssp longum, Bifidobacteria, Bifidobacterium pseudocatenulatum, Bifidobacterium animalis, Bacteroides, Bacteroides ovatus, Ruminococcus, Ruminococcus gnavus, Ruminococcus torques, Blautia, Roseburia, Faecalibacterium, or any combination thereof.

Aspect K18: The method of any one of aspects K1-K17, or any preceding aspect, wherein the method slows microbial utilization of choline.

Aspect K19: The method of any one of aspects K1-K18, or any preceding aspect, wherein the method decreases microbial production of putrescine.

Aspect K₂O: The method of any one of aspects K1-K19, or any preceding aspect, wherein the oligosaccharide composition further comprises at least one microorganism.

Aspect K21: The method of aspect K₂O, or any preceding aspect, wherein the at least one microorganism is modulated by the oligosaccharide composition.

Aspect K22: The method of aspect K₂O or K21, or any preceding aspect, wherein the at least one microorganism is at least one of Lactobacillus crispatus, Lactobacillus Rhamnosus GG, Bifidobacterium Longum ssp Infantis, Bifidobacterium Pseudocatenatum, Bifidobacterium Longum ssp longum, Bifidobacteria, Bifidobacterium pseudocatenulatum, Bifidobacterium animalis, Bacteroides, Bacteroides ovatus, Ruminococcus, Ruminococcus gnavus, Ruminococcus torques, Blautia, Roseburia, Faecalibacterium, or any combination thereof.

Aspect L1: A formulation comprising the oligosaccharide composition, or the one or more oligosaccharides, of any preceding aspect.

Aspect L2: The formulation of aspect L1, or any preceding aspect, further comprising at least one microorganism.

Aspect L3: The formulation of aspect L2, or any preceding aspect, wherein the at least one microorganism comprises at least one of Bifidobacteria, Bifidobacterium pseudocatenulatum, Bifidobacterium animalis, Bacteroides, Bacteroides ovatus, Firmicutes, Clostridium butyricum, Ruminococcus, Ruminococcus gnavus, Ruminococcus torques, Blautia, Roseburia, Faecalibacterium, Lactobacillus crispatus, Lactobacillus rhamnosus, Lactobacillus Rhamnosus GG, Bifidobacterium longum subsp. longum, Bifidobacterium longum subsp. infantis, Ruminococcus torques, or any combination thereof.

Aspect L4: The formulation of any one of aspects L1-L3, or any preceding aspect, further comprising a pharmaceutically acceptable carrier.

Aspect L5: The formulation of any one of aspects L1-L4, or any preceding aspect, wherein the formulation is in the form of a food, a drink, a nutritional supplement, a medicine, an injection, a pill, a capsule, a cream, or a topical ointment.

Aspect M1: A method of generating oligosaccharides from polysaccharides, the method comprising:
reacting polysaccharides in a reaction mixture with hydrogen peroxide and a transition metal or an alkaline earth metal;
quenching the reaction mixture, cleaving glycosidic linkages in the polysaccharides, or both quenching the reaction mixture and cleaving glycosidic linkages in the polysaccharides, wherein the quenching, the cleaving, or both the quenching and the cleaving comprises addition of a base or a non-arrhenius base to the reaction mixture, thereby generating a mixture of oligosaccharides from the polysaccharides.

Aspect M2: The method of aspect M1, or any preceding aspect, wherein the reaction mixture comprises a transition metal.

Aspect M3: The method of aspect M1 or M2, or any preceding aspect, wherein the transition metal comprises iron, Fe³⁺, Fe²⁺, copper, Cu²⁺, manganese, zinc, cobalt, molybdenum, or any combination thereof.

Aspect M4: The method of any one of aspects M1-M3, or any preceding aspect, wherein the reaction mixture comprises an alkaline earth metal.

Aspect M5: The method of any one of aspects M1-M4, or any preceding aspect, wherein the alkaline earth metal comprises calcium, magnesium, or a combination thereof.

Aspect M6: The method of any one of aspects M1-M5, or any preceding aspect, wherein the transition metal in the reaction mixture is at a concentration of at least 0.65 mM (e.g., at least 0.7, at least 0.8, at least 1, at least 2, at least 5, at least 10, at least 15, or at least 18 mM; optionally 20, less than 20, less than 15, less than 10, less than 5, less than 2, less than 1, or less than 0.8 mM and and of these values can be combined in any manner to form a range, such as 0.7 to 5 mM).

Aspect M7: The method of any one of aspects M1-M6, or any preceding aspect, wherein the transition metal in the reaction mixture is at a concentration from 10 μM to 20 mM (e.g., 10 μM to 100 μM, 10 μM to 700 μM, 10 μM to 1 mM, 10 μM to 5 mM, 10 μM to 10 mM, 10 μM to 15 mM, 100 μM to 1 mM, 100 μM to 10 mM, 1 mM to 5 mM, 1 mM to 10 mM, 5 mM to 15 mM, or 10 mM to 20 mM).

Aspect M8: The method of any one of aspects M1-M7, or any preceding aspect, wherein the hydrogen peroxide in the reaction mixture is at a concentration of at least 1 M (e.g., at least 1.5, at least 2, at least 5, or at least 10 M; optionally 15, less than 15, less than 10, less than 5, less than 2, or less than 1.5 M and each of such values can be combined in any manner to form a range, such as 1 to 10 M).

Aspect M9: The method of any one of aspects M1-M8, or any preceding aspect, wherein the hydrogen peroxide in the reaction mixture is at a concentration of from 1 M to 5 M (e.g., 1-2, 1-3, 1-4, 2-3, 2-4, 2-5, 3-4, 3-5, or 4-5 M).

Aspect M10: The method of any one of aspects M1-M9, or any preceding aspect, wherein the base is a nitrogen-based cleavage reagent, optionally the base comprises ammonium hydroxide.

Aspect M11: The method of any one of aspects M1-M10, or any preceding aspect, wherein the base is at a concentration of at least 0.1 M (e.g., at least 0.2, at least 0.5, at least 0.8, at least 1, at least 1.5, at least 2, at least 3, or at least 4 M; optionally 5, less than 5, less than 4, less than 3, less than 2, or less than 1.5 M and each of such values can be combined in any manner to form a range, such as 1.5 to 4).

Aspect M12: The method of any one of aspects M1-M11, or any preceding aspect, wherein the base is at a concentration of from 0.1 M to 5 M.

Aspect M13: The method of any one of aspects M1-M12, or any preceding aspect, wherein the polysaccharides comprise one or more of amylose, amylopectin, betaglucan, pullulan, xyloglucan, arabinogalactan I, arabinogalactan II, rhamnogalacturonan I, rhamnogalacturonan II, galactan, arabinan, arabinoxylan, xylan, glycogen, mannan, glucomannan, curdlan, inulin, pectic galactan or any combination thereof.

Aspect M14: The method of any one of aspects M1-M13, or any preceding aspect, wherein the polysaccharides are in the form of plant material, microorganisms, bacteria, algae, yeast, fungus, or any combination thereof.

Aspect M15: The method of any one of aspects M1-M14, or any preceding aspect, further comprising purifying one or more oligosaccharide from the mixture of oligosaccharides.

Aspect M16: The method of any one of aspects M1-M15, or any preceding aspect, wherein prior to the reacting, the method comprises contacting the polysaccharides with one or more polysaccharide degrading enzymes.

Aspect M17: The method of aspect M16, or any preceding aspect, wherein the one or more polysaccharide degrading enzymes comprises an amylase, an isoamylase, a cellulase, a maltase, a glucanase, or any combination thereof.

Aspect M18: A composition comprising the mixture of oligosaccharides of any one of aspects M1-M17, or any preceding aspect.

Aspect M19: A method of stimulating microbial growth in vitro or in vivo, the method comprising:
contacting microbiota with the composition comprising the mixture of oligosaccharides of aspect M18 under conditions to selectively stimulate growth of the microbiota, optionally wherein the microbiota are probiotic microbiota.

Aspect N1: A synthetic composition comprising one or more oligosaccharides, wherein the one or more oligosaccharides collectively comprise arabinose, galactose, glucose, galacturonic acid, xylose, or rhamnose subunits, or any combination thereof.

Aspect N2: The synthetic composition of aspect N1, or any preceding aspect, wherein at least one oligosaccharide of the one or more oligosaccharides comprises arabinose subunits.

Aspect N3: The synthetic composition of aspect N1 or N2, or any preceding aspect, wherein at least one oligosaccharide of the one or more oligosaccharides consists of arabinose subunits.

Aspect N4: The synthetic composition of any one of aspects N1-N3, or any preceding aspect, wherein at least one oligosaccharide of the one or more oligosaccharides comprises a 5-linked arabinose backbone.

Aspect N5: The synthetic composition of any one of aspects N1-N4, or any preceding aspect, wherein at least one oligosaccharide of the one or more oligosaccharides comprises a 5-linked arabinose backbone with 2-linked branches, 3-linked branches, or a combination thereof.

Aspect N6: The synthetic composition of any one of aspects N1-N5, or any preceding aspect, wherein the arabinose subunits are present, and arabinose linkages are linked in an alpha orientation.

Aspect N7: The synthetic composition of any one of aspects N1-N6, or any preceding aspect, wherein at least one oligosaccharide of the one or more oligosaccharides comprises 3-30 (e.g., 3-4, 3-5, 3-6, 3-8, 3-10, 3-15, 3-20, 3-25, 3-30, 5-10, 5-15, 5-20, 5-25, 5-30, 8-10, 8-15, 8-20, 8-25, 8-30, 10-15, 10-20, 10-25, 10-30, 15-20, 15-25, 15-30, 20-25, 20-30, 25-30, 3-5, 5-8, 8-10, 10-15, 15-20, 20-25, or 25-30) arabinose subunits.

Aspect N8: The synthetic composition of any one of aspects N1-N7, or any preceding aspect, wherein at least one oligosaccharide of the one or more oligosaccharides comprises 3-8 arabinose subunits.

Aspect N9: The synthetic composition of any one of aspects N1-N8, or any preceding aspect, wherein at least one oligosaccharide of the one or more oligosaccharides comprises at least one (e.g., at least 2, at least 3, at least 4, at least 5, at least 6, at least 7; optionally 8 or less, 7 or less, 6 or less, 5 or less, 4 or less, or 3 or less and each of such values can be combined in any manner to form a range, such as 4-8) arabinose subunit and at least one (e.g., at least 2, at least 3, at least 4, at least 5, at least 6, at least 7; optionally 8 or less, 7 or less, 6 or less, 5 or less, 4 or less, or 3 or less and each of such values can be combined in any manner to form a range, such as 4-8) hexose subunit.

Aspect N10: The synthetic composition of aspect N9, or any preceding aspect, wherein the at least one hexose subunit is glucose, galactose, or a combination thereof.

Aspect N11: The synthetic composition of any one of aspects N1-N10, or any preceding aspect, wherein at least one oligosaccharide of the one or more oligosaccharides comprises 3-30 (e.g., 3-4, 3-5, 3-6, 3-8, 3-10, 3-15, 3-20, 3-25, 3-30, 5-10, 5-15, 5-20, 5-25, 5-30, 8-10, 8-15, 8-20, 8-25, 8-30, 10-15, 10-20, 10-25, 10-30, 15-20, 15-25, 15-30, 20-25, 20-30, 25-30, 3-5, 5-8, 8-10, 10-15, 15-20, 20-25, or 25-30) subunits.

Aspect N12: The synthetic composition of any one of aspects N1-N11, or any preceding aspect, wherein at least one oligosaccharide of the one or more oligosaccharides comprises 3-8 (e.g., 3-4, 3-5, 3-6, 3-7, 4-5, 4-6, 4-7, 4-8, 5-6, 5-7, 5-8, 6-7, 6-8, or 7-8) subunits.

Aspect N13: The synthetic composition of any one of aspects N1-N12, or any preceding aspect, wherein at least one oligosaccharide of the one or more oligosaccharides comprises at least one (e.g., at least 2, at least 3, at least 4, at least 5, at least 6, at least 7; optionally 8 or less, 7 or less, 6 or less, 5 or less, 4 or less, or 3 or less and each of such values can be combined in any manner to form a range, such as 4-8) arabinose subunit and up to 7 (e.g., 0, at least 1, at least 2, at least 3, at least 4, at least 5, at least 6, at least 7; optionally 7 or less, 6 or less, 5 or less, 4 or less, 3 or less, 2 or less, or 1 or less and each of such values can be combined in any manner to form a range, such as 4-7) hexose subunits.

Aspect N14: The synthetic composition of any one of aspects N1-N13, or any preceding aspect, wherein the one or more oligosaccharides comprises at least one of:
a. an oligosaccharide analysis comprising the compounds set forth in Table 37, Table 25, Table 29, or Table 30, and wherein each compound in the oligosaccharide analysis is within 10% (e.g., 0-10%, 0-5%, 1-10%, 1-5%, or 1-3%) of the retention time or retention factor and within 30% (e.g., 0-30%, 0-20%, 0-10%, 0-5%, 1-30%, 1-20%, 1-10%, 1-5%, or 1-3%) of the weight percent values set forth in Table 37, Table 25, Table 29, or Table 30;
b. at least 30 wt. % (e.g., at least 50 wt. %, at least 70 wt. %, or at least 90 wt. %; optionally 100 wt. %, less than 100 wt. %, less than 80 wt. %, less than 60 wt. %, or less than 40 wt. % and such values can be combined in any manner to form a range, such as 50-80 wt. %) of the compounds set forth in Table 37, Table 25, Table 29, or Table 30;
c. at least 30 wt. % (e.g., at least 50 wt. %, at least 70 wt. %, or at least 90 wt. %; optionally 100 wt. %, less than 100 wt. %, less than 80 wt. %, less than 60 wt. %, or less than 40 wt. % and such values can be combined in any manner to form a range, such as 30-60 wt. %) of the following compounds (name, retention time (minutes); respectively): (3 hexoses, 3.24), (2 hexoses and 1 pentose, 3.76), (4 hexoses, 8.28), (3 hexoses and 1 pentose, 9.02), (5 hexoses, 11.56), and (4 hexoses and 1 pentose, 12.045);
d. any combination thereof.

Aspect N15: The synthetic composition of any one of aspects N1-N14, or any preceding aspect, wherein the one or more oligosaccharides have, or the synthetic composition has, an dynmatic viscosity of between 1-6 mPa*s (e.g., 1-2, 1-3, 1-4, 1-5, 2-3, 2-4, 2-5, 2-6, 3-4, 3-5, 3-6, 4-5, 4-6, or 5-6 mPa*s) at a concentration of 100 mg/ml in water at 25° C.

Aspect N16: The synthetic composition of any one of aspects N1-N15, or any preceding aspect, wherein the synthetic composition collectively comprises arabinose, xylose, galactose, glucose, galacturonic acid, or rhamnose subunits, or any combination thereof, as determined by hydrolytic monosaccharide compositional analysis of the synthetic composition.

Aspect N17: The synthetic composition of any one of aspects N1-N16, or any preceding aspect, wherein the synthetic composition collectively comprises arabinose, galactose, and glucose subunits, as determined by hydrolytic monosaccharide compositional analysis of the synthetic composition.

Aspect N18: The synthetic composition of any one of aspects N1-N17, or any preceding aspect, wherein the synthetic composition collectively comprises arabinose, as determined by hydrolytic monosaccharide compositional analysis of the synthetic composition.

Aspect N19: The synthetic composition of any one of aspects N1-N18, or any preceding aspect, wherein the synthetic composition collectively comprises 5-linked, 3,5-linked, 2,5-linked, 2,3,5-linked, and terminal arabinose subunits, as determined by glycosidic linkage analysis of the synthetic composition.

Aspect N20: The synthetic composition of any one of aspects N1-N19, or any preceding aspect, wherein, collectively, at least 90% (e.g., at least 92%, at least 94%, at least 95%, at least 96%, at least 98%, at least 99%, or 100%; optionally less than 100%, less than 99%, less than 98%, less than 96%, less than 94%, or less than 92% and each of such values can be combined in any manner to form a range, such as 95-99%) of the arabinose glycosidic linkages are in an alpha orientation, as determined by NMR HSQC analysis of the synthetic composition.

Aspect N21: The synthetic composition of any one of aspects N1-N20, or any preceding aspect, wherein the synthetic composition collectively comprises at least one of the following, as determined by glycosidic linkage analysis of the synthetic composition:
a. a terminal arabinose to 5-linked arabinose linkage ratio of between 10:1 and 0.1:1 (e.g., 0.1:1 to 5:1, 0.5:1 to 4:1, 1:1 to 3:1, or 1.2:1 to 2.5:1) or about 1.5:1 or about 1.3:1 or about 2.2:1;
b. a terminal arabinose to 3,5-linked arabinose linkage ratio of between 1:1 and 15:1 (e.g., 2:1 to 10:1, 2.5:1 to 8.5:1, 7:1 to 9:1, 2:1 to 4:1, or 3:1 to 5:1) or about 8.3:1 or about 2.9:1 or about 9.2:1 or about 3.5:1;
c. a terminal arabinose to 2,5-linked arabinose linkage ratio of between 1:1 and 15:1 (e.g., 3:1 to 12:1, 5:1 to 9:1, 3:1 to 5:1, 9:1 to 12:1, or 5:1 to 9:1) or about 3.8:1 or about 10.7:1 or about 7.4:1 or about 7.7:1;
d. a terminal arabinose to 2,3,5-linked arabinose linkage ratio of between 1:1 and 20:1 (e.g., 4:1 to 16:1, or 8:1 to 12:1, or 7:1 to 9:1, or 5:1 to 8:1, 13:1 to 16:1, or 6.5:1 to 9:1) or about 8:1 or about 6.4:1 or about 14.4:1 or about 7.7:1;
e. any combination thereof.

Aspect N22: The synthetic composition of any one of aspects N1-N21, or any preceding aspect, wherein the synthetic composition collectively comprises 4-linked and terminal galactose, as determined by glycosidic linkage analysis of the synthetic composition.

Aspect N23: The synthetic composition of any one of aspects N1-N22, or any preceding aspect, wherein the synthetic composition collectively comprises a 4-linked galactose to terminal galactose linkage ratio of between 0.1:1 and 15:1 (e.g., 0.5:1 to 10:1, 1:1 to 8:1, 0.5:1 to 3:1, 7:1 to 9:1, or 3:1 to 5:1) or about 1.6:1 or about 7.9:1 or about 3.9:1, as determined by glycosidic linkage analysis of the synthetic composition.

Aspect N24: The synthetic composition of any one of aspects N1-N23, or any preceding aspect, wherein, collectively, at least 90% (e.g., at least 92%, at least 94%, at least 95%, at least 96%, at least 98%, at least 99%, or 100%; optionally less than 100%, less than 99%, less than 98%, less than 96%, less than 94%, or less than 92% and each of such values can be combined in any manner to form a range, such as 95-99%) of galactose glycosidic linkages are in a beta orientation, as determined by NMR HSQC analysis of the synthetic composition.

Aspect N25: The synthetic composition of any one of aspects N1-N24, or any preceding aspect, wherein the synthetic composition collectively comprises at least 30% (e.g., at least 50%, at least 70%, or at least 90%, or about 100%; optionally 100%, less than 100%, less than 80%, less than 60%, or less than 40% and such values can be combined in any manner to form a range, such as 50-100%) arabinose, as determined by hydrolytic monosaccharide compositional analysis of the synthetic composition.

Aspect N26: The synthetic composition of any one of aspects N1-N25, or any preceding aspect, wherein the synthetic composition collectively comprises at least one of the following, as determined by hydrolytic monosaccharide compositional analysis of the synthetic composition:
a. an arabinose to galactose monosaccharide ratio of between 50:1 and 0.1:1 (e.g., 1:1 to 20:1, 1:1 to 4:1, 7:1 to 10:1, 12:1 to 16:1, 0.5:1 to 1:1, or 1.5:1 to 3:1) or about 8.4:1 or about 14:1 or about 0.7:1 or about 2.2:1;
b. an arabinose to rhamnose monosaccharide ratio of between 10:1 and 50:1 (e.g., 10:1 to 35:1, or 28:1 to 34:1, 12:1 to 16:2, or 24:1 to 28:1) or about 32.2:1 or about 14.1:1 or about 26.6:1;
c. an arabinose to glucose monosaccharide ratio of between 1:1 to 50:1 (e.g., 3:1 to 25:1, 18:1 to 22:1, 10:1 to 14:1, 3:1 to 5:1, or 6:1 to 9:1) or about 19.9:1 or about 12.3:1 or about 4.2:1 or about 7.9:1;
d. an arabinose to galacturonic acid monosaccharide ratio of between 5:1 to 30:1 (e.g., 8:1 to 25:1, 20:1 to 24:1, 9:1 to 13:1, or 20:1 to 26:1) or about 22.5:1 or about 11.6:1 or about 23.6:1;
e. any combination thereof.

Aspect N27: The synthetic composition of any one of aspects N1-N26, or any preceding aspect, wherein the synthetic composition, as determined by NMR HSQC analysis, collectively comprises at least one 2D NMR correlation that is within 0.1 ppm (e.g., within 0.05 ppm) of a 1H coordinate and within 3.0 ppm (e.g., within 2.5, within 2, within 1.5, or within 1 ppm) of a 13C coordinate for at least one of the following:
a. one or more 2D NMR correlations set forth in Table O for CLX122 or CLX122-DS or CLX126 or CLX127;
b. one or more 2D NMR correlations shown in FIG. 41A or FIG. 41B or FIG. 41F or FIG. 42A;
c. one or more of 2D NMR correlations as follows (1H ppm, 13C ppm): (5.00, 107.20), (4.94, 107.81), (4.86, 107.68), (4.84, 107.20), (4.04, 85.82), (3.98, 80.77), (3.93, 88.01), (3.71, 67.24), (3.55, 67.11), or any combination thereof;
d. any combination thereof.

Aspect N28: The synthetic composition of any one of aspects N1-N27, or any preceding aspect, wherein the synthetic composition is or comprises CLX122 or CLX122-DS or CLX126 or CLX127.

Aspect N29: The synthetic composition of any one of aspects N1-N28, or any preceding aspect, wherein the synthetic composition is derived from at least one material comprising pea, soy, carrot, or any combination thereof.

Aspect N30: The synthetic composition of aspect N29, or any preceding aspect, wherein the synthetic composition is derived from the at least one material comprising pea, soy, carrot, or any combination thereof by a process comprising:
a. reacting the at least one material in a reaction mixture with hydrogen peroxide and a transition metal or an alkaline earth metal; and
b. quenching the reaction mixture, cleaving glycosidic linkages in the at least one material, or both quenching the reaction mixture and cleaving glycosidic linkages in the at least one material, wherein the quenching, the cleaving, or both the quenching and the cleaving comprise(s) addition of a base or a non-arrhenius base to the reaction mixture, thereby generating the synthetic composition from the at least one material.

Aspect N31: The synthetic composition of any one of aspects N1-N30, or any preceding aspect, wherein, when a microbial community comprising Bifidobacterium and Bacteroides intestinales is subjected to the synthetic composition, an abundance of Bifidobacterium is increased and an abundance of Bacteroides intestinales is decreased, as compared to an otherwise identical community that is not subjected to the synthetic composition.

Aspect N32: The synthetic composition of any one of aspects N1-N31, or any preceding aspect, wherein, when a microbial community comprising Blautia and Proteobacteria is subjected to the synthetic composition, an abundance of Blautia is increased and an abundance of Proteobacteria is decreased, as compared to an otherwise identical community that is not subjected to the synthetic composition.

Aspect N33: The synthetic composition comprising any one of aspects N1-N32, or any preceding aspect, further comprising at least one microorganism.

Aspect N34: The synthetic composition of aspects N1-N33, or any preceding aspect, wherein the at least one microorganism comprises Bifidobacterium, Bifidobacterium pseudocatenulatum, Bifidobacterium longum subsp. longum, Bacteroides, Bacteroides ovatus, Lactobacillus, Clostridia, Clostridium butyricum, Ruminococcus, Ruminococcus gnavus, Ruminococcus torques, Blautia, Roseburia, Faecalibacterium, or any combination thereof.

Aspect N35: The synthetic composition of aspect N33 or N34, or any preceding aspect, wherein the synthetic composition is capable of modulating the at least one microorganism.

Aspect N36: A method for modulating a microbial community comprising at least one microorganism, the method comprising contacting the microbial community with the synthetic composition of any one of aspects N1-N35, or any preceding aspect, wherein the at least one microorganism is modulated.

Aspect N37: The method of aspect N36, or any preceding aspect, wherein the microbial community is located in a vaginal tract, a gut, a respiratory system, an oral cavity, an eye, on skin, or any combination thereof.

Aspect N38: The method of aspect N36 or N37, or any preceding aspect, wherein the at least one microorganism comprises Bifidobacterium, and wherein the method increases an abundance of the Bifidobacterium.

Aspect N39: The method of aspect N38, or any preceding aspect, or any preceding aspect, wherein the Bifidobacterium comprises Bifidobacterium pseudocatenulatum, Bifidobacterium longum subsp. longum, or a combination thereof, and the method increases an abundance of the Bifidobacterium pseudocatenulatum, Bifidobacterium longum subsp. longum, or combination thereof.

Aspect N40: The method of any one of aspects N36-N39, or any preceding aspect, wherein the at least one microorganism comprises Bacteroides, and wherein the method increases an abundance of Bacteroides.

Aspect N41: The method of aspect N40, or any preceding aspect, wherein the Bacteroides comprises Bacteroides ovatus, Bacteroides vulgatus, or a combination thereof, and wherein the method increases an abundance of the Bacteroides ovatus, Bacteroides vulgatus, or combination thereof.

Aspect N42: The method of any one of aspects N36-N41, or any preceding aspect, wherein the at least one microorganism comprises Clostridia, and wherein the method increases an abundance of the Clostridia.

Aspect N43: The method of aspect N42, or any preceding aspect, wherein the Clostridia comprises Clostridium butyricum, and wherein the method increases an abundance of the Clostridium butyricum.

Aspect N44: The method of any one of aspects N36-N43, or any preceding aspect, wherein the at least one microorganism comprises Blautia, Roseburia, Faecalibacterium, or any combination thereof, and wherein the method increases an abundance of the Blautia, Roseburia, Faecalibacterium, or any combination thereof.

Aspect N45: The method of any one of aspects N36-N44, or any preceding aspect, wherein the at least one microorganism comprises Ruminococcus, and wherein the method increases an abundance of the Ruminococcus.

Aspect N46: The method of aspect N45, or any preceding aspect, wherein the Ruminococcus comprises Ruminococcus gnavus, Ruminococcus torques, or a combination thereof, and wherein the method increases an abundance of the Ruminococcus gnavus, Ruminococcus torques, or combination thereof.

Aspect N47: The method of any one of aspects N36-N46, or any preceding aspect, wherein the at least one microorganism comprises Proteobacteria, and wherein the method decreases an abundance of the Proteobacteria.

Aspect N48: The method of any one of aspects N36-N47, or any preceding aspect, wherein the at least one microorganism comprises Parabacteroides distasonis, and wherein the method decreases an abundance of the Parabacteroides distasonis.

Aspect N49: The method of any one of aspects N36-N48, or any preceding aspect, wherein the at least one microorganism comprises Bacteroides intestinalis, and wherein the method decreases an abundance of the Bacteroides intestinalis.

Aspect N50: The method of any one of aspects N36-N49, or any preceding aspect, wherein the modulating comprises enhancing production by the at least one microorganism of at least one short chain fatty acid optionally selected from butyrate, lactate, or a combination thereof.

Aspect N51: The method of any one of aspects N36-N50, or any preceding aspect, wherein the synthetic composition further comprises an additional microorganism, wherein the synthetic composition is capable of modulating the additional microorganism, and optionally wherein the at least one microorganism and the additional microorganism are the same genus, species, or subspecies.

Aspect N52: A synthetic composition comprising one or more oligosaccharides, wherein the one or more oligosaccharides collectively comprise arabinose, glucose, galactose, and xylose, or any combination thereof.

Aspect N53: The synthetic composition of aspect N52, or any preceding aspect, wherein at least one oligosaccharide of the one or more oligosaccharides comprises xylose subunits.

Aspect N54: The synthetic composition of aspect N52 or N53, or any preceding aspect, wherein at least one oligosaccharide of the one or more oligosaccharides consists of one or more arabinose subunits.

Aspect N55: The synthetic composition of any one of aspects N52-N54, or any preceding aspect, wherein the one or more oligosaccharides collectively comprise less than 5% 4-o-methylated glucuronic acid.

Aspect N56: The synthetic composition of any one of aspects N52-N55, or any preceding aspect, wherein at least one oligosaccharide of the one or more oligosaccharides contain both xylose and arabinose subunits.

Aspect N57: The synthetic composition of any one of aspects N52-N56, or any preceding aspect, wherein at least one oligosaccharide of the one or more oligosaccharides comprises 5-linked or terminal arabinose units, or a combination thereof.

Aspect N58: The synthetic composition of any one of aspects N52-N57, or any preceding aspect, wherein the arabinose subunits are present, and arabinose linkages are linked in an alpha orientation.

Aspect N59: The synthetic composition of any one of aspects N52-N58, or any preceding aspect, wherein at least one oligosaccharide of the one or more oligosaccharides comprises a 4-linked xylose backbone or a 3-linked xylose branch or terminal xylose units, or a combination thereof.

Aspect N60: The synthetic composition of any one of aspects N52-N59, or any preceding aspect, wherein at least one oligosaccharide of the one or more oligosaccharides comprises a xylose backbone with a 3-linked branch to a galactose or arabinose, or a combination thereof.

Aspect N61: The synthetic composition of any one of aspects N52-N60, or any preceding aspect, wherein the xylose subunits are present, and xylose linkages are linked in a beta orientation.

Aspect N62: The synthetic composition of any one of aspects N52-N61, or any preceding aspect, wherein at least one oligosaccharide of the one or more oligosaccharides comprises galactose as a terminal unit.

Aspect N63: The synthetic composition of any one of aspects N52-N62, or any preceding aspect, wherein at least one oligosaccharide of the one or more oligosaccharides comprises 3-30 (e.g., 3-4, 3-5, 3-6, 3-8, 3-10, 3-15, 3-20, 3-25, 3-30, 5-10, 5-15, 5-20, 5-25, 5-30, 8-10, 8-15, 8-20, 8-25, 8-30, 10-15, 10-20, 10-25, 10-30, 15-20, 15-25, 15-30, 20-25, 20-30, 25-30, 3-5, 5-8, 8-10, 10-15, 15-20, 20-25, or 25-30) pentose subunits.

Aspect N64: The synthetic composition of any one of aspects N52-N63, or any preceding aspect, wherein at least one oligosaccharide of the one or more oligosaccharides comprises 3-7 (e.g., 3-4, 3-5, 3-6, 4-5, 4-6, 4-7, 5-6, 5-7, or 6-7) pentose subunits.

Aspect N65: The synthetic composition of any one of aspects N52-N64, or any preceding aspect, wherein at least one oligosaccharide of the one or more oligosaccharides comprises at least one (e.g., at least 2, at least 3, at least 4, at least 5, at least 6, at least 7; optionally 8 or less, 7 or less, 6 or less, 5 or less, 4 or less, or 3 or less and each of such values can be combined in any manner to form a range, such as 4-8) pentose subunit and at least one (e.g., at least 2, at least 3, at least 4, at least 5, at least 6, at least 7; optionally 8 or less, 7 or less, 6 or less, 5 or less, 4 or less, or 3 or less and each of such values can be combined in any manner to form a range, such as 4-8) hexose subunit.

Aspect N66: The synthetic composition of any one of aspects N52-N65, or any preceding aspect, wherein at least one oligosaccharide of the one or more oligosaccharides comprises at least one (e.g., at least 2, at least 3, at least 4, at least 5, at least 6, at least 7; optionally 8 or less, 7 or less, 6 or less, 5 or less, 4 or less, or 3 or less and each of such values can be combined in any manner to form a range, such as 4-8) pentose subunit at up to four (e.g., 0, at least 1, at least 2, or at least 3; optionally 4 or less, 3 or less, 2 or less, or 1 or less and each of such values can be combined in any manner to form a range, such as 1-4) hexose subunits.

Aspect N67: The synthetic composition of aspects N52-N66, or any preceding aspect, wherein the at least one hexose subunit is glucose, galactose, or a combination thereof.

Aspect N68: The synthetic composition of aspects N52-N67, or any preceding aspect, wherein the at least one pentose subunit is arabinose, xylose, or a combination thereof.

Aspect N69: The synthetic composition of any one of aspects N52-N68, or any preceding aspect, wherein the one or more oligosaccharides comprises at least one of:
a. an oligosaccharide analysis comprising the compounds set forth in Table 31 and wherein each compound in the oligosaccharide analysis is within 10% (e.g., 0-10%, 0-5%, 1-10%, 1-5%, or 1-3%) of the retention time or retention factor and within 30% (e.g., 0-30%, 0-20%, 0-10%, 0-5%, 1-30%, 1-20%, 1-10%, 1-5%, or 1-3%) of the weight percent values set forth in Table 31;
b. at least 30 wt. % (e.g., at least 50 wt. %, at least 70 wt. %, or at least 90 wt. %; optionally 100 wt. %, less than 100 wt. %, less than 80 wt. %, less than 60 wt. %, or less than 40 wt. % and such values can be combined in any manner to form a range, such as 50-80 wt. %) of the compounds set forth in Table 31;
c. at least 30 wt. % (e.g., at least 50 wt. %, at least 70 wt. %, or at least 90 wt. %; optionally 100 wt. %, less than 100 wt. %, less than 80 wt. %, less than 60 wt. %, or less than 40 wt. % and such values can be combined in any manner to form a range, such as 50-80 wt. %) of the following compounds (name, retention time (minutes); respectively): (3 hexoses, 3.24), (2 hexoses and 1 pentose, 3.76), (4 hexoses, 8.28), (3 hexoses and 1 pentose, 9.02), (5 hexoses, 11.56), and (4 hexoses and 1 pentose, 12.045);
d. any combination thereof.

Aspect N70: The synthetic composition of any one of aspects N52-N69, or any preceding aspect, wherein the one or more oligosaccharides have, or the synthetic composition has, an dynamic viscosity of between 1-4 mPa*s (e.g., 1-2, 1-3, 2-3, 2-4, or 3-4) at a concentration of 100 mg/ml in water at 25° C.

Aspect N71: The synthetic composition of any one of aspects N52-N70, or any preceding aspect, wherein the synthetic composition collectively comprises arabinose, xylose, galactose, or glucose subunits, or any combination thereof, as determined by hydrolytic monosaccharide compositional analysis of the synthetic composition.

Aspect N72: The synthetic composition of any one of aspects N52-N71, or any preceding aspect, wherein the synthetic composition collectively comprises arabinose, galactose, and xylose subunits, as determined by hydrolytic monosaccharide compositional analysis of the synthetic composition.

Aspect N73: The synthetic composition of any one of aspects N52-N72, or any preceding aspect, wherein the synthetic composition collectively comprises xylose and glucose, as determined by hydrolytic monosaccharide compositional analysis of the synthetic composition.

Aspect N74: The synthetic composition of any one of aspects N52-N73, or any preceding aspect, wherein the synthetic composition collectively comprises xylose and arabinose, as determined by hydrolytic monosaccharide compositional analysis of the synthetic composition.

Aspect N75: The synthetic composition of any one of aspects N52-N74, or any preceding aspect, wherein the synthetic composition collectively comprises xylose, as determined by hydrolytic monosaccharide compositional analysis of the synthetic composition.

Aspect N76: The synthetic composition of any one of aspects N52-N75, or any preceding aspect, wherein the synthetic composition collectively comprises 4-linked, 3,4-linked, and terminal xylose subunits, as determined by glycosidic linkage analysis of the synthetic composition.

Aspect N77: The synthetic composition of any one of aspects N52-N76, or any preceding aspect, wherein, collectively, at least 90% (e.g., at least 92%, at least 94%, at least 95%, at least 96%, at least 98%, at least 99%, or 100%; optionally less than 100%, less than 99%, less than 98%, less than 96%, less than 94%, or less than 92% and each of such values can be combined in any manner to form a range, such as 95-99%) of the xylose glycosidic linkages are in a beta orientation, as determined by NMR HSQC analysis of the synthetic composition.

Aspect N78: The synthetic composition of any one of aspects N52-N77, or any preceding aspect, wherein the synthetic composition collectively comprises at least one of the following, as determined by glycosidic linkage analysis of the synthetic composition:
a. a 4-linked xylose to 3,4-linked xylose linkage ratio of between 1:1 and 10:1 (e.g., 1:1 to 3:1, 1:1 to 6:1, 1:1 to 8:1, 2:1 to 6:1, or 4.5:1 to 6.5:1) or about 5.6:1;
b. a 4-linked xylose to terminal xylose linkage ratio of between 1:1 and 9:1 (e.g., 2:1 to 8:1, 3:1 to 6:1, or 4:1 to 5:1) or about 4.6:1;
c. any combination thereof.

Aspect N79: The synthetic composition of any one of aspects N52-N78, or any preceding aspect, wherein the synthetic composition collectively comprises 5-linked and terminal arabinose, as determined by glycosidic linkage analysis of the synthetic composition.

Aspect N80: The synthetic composition of any one of aspects N52-N79, or any preceding aspect, wherein the synthetic composition collectively comprises a terminal arabinose to 5-linked arabinose linkage ratio of between 0.1:1 and 8:1 (e.g., 0.1:1 to 4:1, or 0.5:1 to 2:1) or about 1.2:1, as determined by glycosidic linkage analysis of the synthetic composition.

Aspect N81: The synthetic composition of any one of aspects N52-N80, or any preceding aspect, wherein, collectively, at least 90% (e.g., at least 92%, at least 94%, at least 95%, at least 96%, at least 98%, at least 99%, or 100%; optionally less than 100%, less than 99%, less than 98%, less than 96%, less than 94%, or less than 92% and each of such values can be combined in any manner to form a range, such as 95-99%) of arabinose glycosidic linkages are in an alpha orientation, as determined by NMR HSQC analysis of the synthetic composition.

Aspect N82: The synthetic composition of any one of aspects N52-N81, or any preceding aspect, wherein the synthetic composition collectively comprises 4-linked, 3,-linked, and terminal glucose subunits, as determined by glycosidic linkage analysis of the synthetic composition.

Aspect N83: The synthetic composition of any one of aspects N52-N82, or any preceding aspect, wherein the synthetic composition collectively comprises at least one of the following, as determined by glycosidic linkage analysis of the synthetic composition:
a. a 4-linked glucose to 3-linked glucose linkage ratio of between 1:1 and 10:1 (e.g., 1:1 to 3:1, 1:1 to 6:1, 1:1 to 8:1, 2:1 to 6:1, or 4.5:1 to 6.5:1) or about 4.5:1;
b. a 4-linked glucose to terminal glucose linkage ratio of between 0.5:1 and 5:1 (e.g., 0.5:1 to 3:1, 0.8:1 to 2.5:1, or 1:1 to 2:1) or about 1.7:1;
c. a 3-linked glucose to terminal glucose linkage ratio between 0.1:1 and 4:1 (e.g., 0.1:1 to 2:1, 0.1:1 to 3:1, or 0.2:1 to 1:1) or 0.4:1;
d. any combination thereof.

Aspect N84: The synthetic composition of any one of aspects N52-N83, or any preceding aspect, wherein, collectively, at least 80% (e.g., at least 85%, at least 90%, at least 95%, at least 99% or 100%; optionally 100%, less than 100%, less than 99%, less than 95%, less than 90%, or less than 85% and each of such values can be combined in any manner to form a range, such as 85-100%) of glucose glycosidic linkages are in a beta orientation, as determined by NMR HSQC analysis of the synthetic composition.

Aspect N85: The synthetic composition of any one of aspects N52-N84, or any preceding aspect, wherein, collectively, at least 85% (e.g., at least 90%, at least 95%, at least 99% or 100%; optionally 100%, less than 100%, less than 99%, less than 95%, or less than 90% and each of such values can be combined in any manner to form a range, such as 95-99%) of the 4-linked glucose glycosidic linkages are in a beta orientation, as determined by NMR HSQC analysis of the synthetic composition.

Aspect N86: The synthetic composition of any one of aspects N52-N85, or any preceding aspect, wherein, collectively, at least 90% (e.g., at least 92%, at least 94%, at least 95%, at least 96%, at least 98%, at least 99%, or 100%; optionally less than 100%, less than 99%, less than 98%, less than 96%, less than 94%, or less than 92% and each of such values can be combined in any manner to form a range, such as 94-99%) of the 3-linked glucose glycosidic linkages are in a beta orientation, as determined by NMR HSQC analysis of the synthetic composition.

Aspect N87: The synthetic composition of any one of aspects N52-N86, or any preceding aspect, wherein the synthetic composition collectively comprises at least 30% (e.g., at least 50 wt. %, at least 70 wt. %, at least 90 wt. %, or about 100%; optionally less than 100 wt. %, less than 80 wt. %, less than 60 wt. %, or less than 40 wt. % and such values can be combined in any manner to form a range, such as 80 wt. % to less than 100 wt. %) xylose, as determined by hydrolytic monosaccharide compositional analysis of the synthetic composition.

Aspect N88: The synthetic composition of any one of aspects N52-N87, or any preceding aspect, wherein the synthetic composition collectively comprises at least one of the following:
a. a xylose to glucose monosaccharide ratio of between 0.5:1 and 10:1 (e.g., 0.5:1 to 6:1, 0.5:1 to 4:1, 0.8:1 to 3:1, or 1:1 to 2:1) or about 1.3:1, as determined by hydrolytic monosaccharide compositional analysis of the synthetic composition;
b. a xylose to arabinose monosaccharide ratio of between 1:1 and 10:1 (e.g., 1:1 to 8:1, 2:1 to 6:1, 2:1 to 5:1, or 3:1 to 5:1) or about 3.9:1, as determined by hydrolytic monosaccharide compositional analysis of the synthetic composition;
c. a xylose to galactose monosaccharide ratio of between 5:1 and 100:1 (e.g., 5:1 to 80:1, 10:1 to 50:1, 10:1 to 30:1, or 15:1 to 25:1) or about 20.7:1, as determined by hydrolytic monosaccharide compositional analysis of the synthetic composition;
d. any combination thereof.

Aspect N89: The synthetic composition of any one of aspects N52-N88, or any preceding aspect, wherein the synthetic composition, as determined by NMR HSQC analysis, collectively comprises at least one 2D NMR correlation that is within 0.1 ppm of a 1H coordinate and within 3.0 ppm of a 13C coordinate for at least one of the following:
a. one or more 2D NMR correlations set forth in Table O for CLX128;
b. one or more 2D NMR correlations shown in FIG. 42B;
c. one or more of 2D NMR correlations as follows (1H ppm, 13C ppm): (5.34, 107.61), (5.21, 107.95), (5.08, 97.84), (4.37, 103.10), (4.26, 101.32), (3.97, 86.71), (3.92, 70.66), (3.87, 63.69), (3.85, 71.68), or any combination thereof;
d. any combination thereof.

Aspect N90: The synthetic composition of any one of aspects N52-N89, or any preceding aspect, wherein the synthetic composition is or comprises CLX128.

Aspect N91: The synthetic composition of any one of aspects N52-N90, or any preceding aspect, wherein the synthetic composition is derived from at least one material comprising sugar cane.

Aspect N92: The synthetic composition of aspect N91, or any preceding aspect, wherein the synthetic composition is derived from the at least one material comprising sugar cane by a process comprising:
a. reacting the at least one material in a reaction mixture with hydrogen peroxide and a transition metal or an alkaline earth metal; and
b. quenching the reaction mixture, cleaving glycosidic linkages in the at least one material, or both quenching the reaction mixture and cleaving glycosidic linkages in the at least one material, wherein the quenching, the cleaving, or both the quenching and the cleaving comprise(s) addition of a base or a non-arrhenius base to the reaction mixture, thereby generating the synthetic composition from the at least one material.

Aspect N93: The synthetic composition of any one of aspects N52-N92, or any preceding aspect, wherein, when a microbial community comprising Bifidobacterium and Proteobacteria is subjected to the synthetic composition, an abundance of Bifidobacterium is increased and an abundance of Proteobacteria is decreased, as compared to an otherwise identical community that is not subjected to the synthetic composition.

Aspect N94: The synthetic composition of any one of aspects N52-N93, or any preceding aspect, wherein, when a microbial community comprising Ruminococcus gnavus and Bacteroides intestinalis is subjected to the synthetic composition, an abundance of Ruminococcus gnavus is increased and an abundance of Bacteroides intestinalis is decreased, as compared to an otherwise identical community that is not subjected to the synthetic composition.

Aspect N95: The synthetic composition comprising any one of aspects N52-N94, or any preceding aspect, further comprising at least one microorganism.

Aspect N96: The synthetic composition of aspects N52-N95, or any preceding aspect, wherein the at least one microorganism comprises Bifidobacterium, Bifidobacterium pseudocatenulatum, Bifidobacterium longum subsp. longum, Bacteroides, Bacteroides ovatus, Lactobacillus, Clostridia, Clostridium butyricum, Ruminococcus, Ruminococcus gnavus, Ruminococcus torques, Blautia, Roseburia, Faecalibacterium, and combinations thereof.

Aspect N97: The synthetic composition of aspects N52-N96, or any preceding aspect, wherein the synthetic composition is capable of modulating the at least one microorganism.

Aspect N98: A method for modulating a microbial community comprising at least one microorganism, the method comprising contacting the microbial community with the synthetic composition of any one of aspects N52-N97, or any preceding aspect, wherein the at least one microorganism is modulated.

Aspect N99: The method of aspect N98, or any preceding aspect, wherein the microbial community is located in a vaginal tract, a gut, a respiratory system, an oral cavity, an eye, on skin, or any combination thereof.

Aspect N100: The method of aspect N98 or N99, or any preceding aspect, wherein the at least one microorganism comprises Bifidobacterium, and wherein the method increases an abundance of the Bifidobacterium.

Aspect N101: The method of aspect N100, or any preceding aspect, wherein the Bifidobacterium comprises Bifidobacterium pseudocatenulatum, Bifidobacterium longum subsp. longum, Bifidobacterium longum subsp. infantis or a combination thereof, and the method increases an abundance of the Bifidobacterium pseudocatenulatum, Bifidobacterium longum subsp. longum, Bifidobacterium longum subsp. infantis or a combination thereof, or combination thereof.

Aspect N102: The method of any one of aspects N98-N101, or any preceding aspect, wherein the at least one microorganism comprises Lactobacillus, and wherein the method increases an abundance of Lactobacillus.

Aspect N103: The method of any one of aspects N98-N102, or any preceding aspect, wherein the at least one microorganism comprises Clostridia, and wherein the method increases an abundance of the Clostridia.

Aspect N104: The method of aspect N103, or any preceding aspect, wherein the Clostridia comprises Clostridium butyricum, and wherein the method increases an abundance of the Clostridium butyricum.

Aspect N105: The method of any one of aspects N98-N104, or any preceding aspect, wherein the at least one microorganism comprises Blautia, Roseburia, Faecalibacterium, or any combination thereof, and wherein the method increases an abundance of the Blautia, Roseburia, Faecalibacterium, or any combination thereof.

Aspect N106: The method of any one of aspects N98-N105, or any preceding aspect, wherein the at least one microorganism comprises Ruminococcus, and wherein the method increases an abundance of the Ruminococcus.

Aspect N107: The method of aspect N106, or any preceding aspect, wherein the Ruminococcus comprises Ruminococcus gnavus, Ruminococcus torques, or a combination thereof, and wherein the method increases an abundance of the Ruminococcus gnavus, Ruminococcus torques, or combination thereof.

Aspect N108: The method of any one of aspects N98-N107, or any preceding aspect, wherein the at least one microorganism comprises Proteobacteria, and wherein the method decreases an abundance of the Proteobacteria.

Aspect N109: The method of any one of aspects N98-N108, or any preceding aspect, wherein the at least one microorganism comprises Parabacteroides distasonis, and wherein the method decreases an abundance of the Parabacteroides distasonis.

Aspect N110: The method of any one of aspects N98-N109, or any preceding aspect, wherein the at least one microorganism comprises Bacteroides, and wherein the method decreases an abundance of the Bacteroides.

Aspect N111: The method of any one of aspects N98-N110, or any preceding aspect, wherein the at least one microorganism comprises Bacteroides ovatus, and wherein the method decreases an abundance of the Bacteroides ovatus.

Aspect N112: The method of any one of aspects N98-N111, or any preceding aspect, wherein the modulating comprises enhancing production by the at least one microorganism of at least one short chain fatty acid optionally selected from butyrate, lactate, or a combination thereof.

Aspect N113: The method of any one of aspects N98-N112, or any preceding aspect, wherein the synthetic composition further comprises an additional microorganism, wherein the synthetic composition is capable of modulating the additional microorganism, and optionally wherein the at least one microorganism and the additional microorganism are the same genus, species, or subspecies.

Aspect N114: A method for treating or preventing a gastrointestinal condition or disease, the method comprising administering to a patient in need thereof an amount, or a therapeutically effective amount, of the synthetic composition of any one of aspects N1-N35 or N52-N97, or any preceding aspect, optionally wherein the synthetic composition is in the form of a pharmaceutical composition comprising a pharmaceutically acceptable carrier.

Aspect N115: The method of aspect N114, or any preceding aspect, wherein the gastrointestinal condition or disease comprises inflammatory bowel disease, Crohn's disease, ulcerative colitis, or any combination thereof.

The invention can be further understood by the following non-limiting examples.

### Example 1

(Ammonium Hydroxide and Ammonium Bicarbonate were Used as Exemplary Polysaccharide (PS)-Cleavage Reagents)

Locust bean gum is known to be high in galactomannan polysaccharides. Galactomannan is a polysaccharide that contains a β1-4-linked mannose backbone with a 1-6-linked galactose branches. Galactomannan (or oligosaccharides derivable therefrom using the disclosed methods) may act to selectively promote the growth of bacteria that can depolymerize one or both of these glycosidic bonds.

Production of Oligosaccharides. In a first exemplary aspect, Locust bean gum (500 mg) was dissolved in 20 ml of HPLC grade water in a capped reaction vessel and placed in a shaker-incubator for 10 min at 55° C. and 85 RPM. The pH of the solution was adjusted to 5.2 with ammonium bicarbonate (0.5 M). Hydrogen peroxide (5 ml) and iron (III) sulfate (2.75 mg in 50 μL water) were added to the reaction mixture and mixed thoroughly. The reaction in the capped reaction vessel was allowed to proceed in the shaker-incubator at 55° C. and 75 RPM for two hours. The capped reaction was allowed to cool to 20° C. Four cleavage conditions were conducted: ammonium hydroxide (1 ml of 28% v/v to pH 10), sodium hydroxide (65 μl, 10.45 M NaOH to pH 10), and two concentrations of ammonium bicarbonate (1.125 g and 5 g, both to pH 7.5). All four conditions were reacted at two temperatures, 27° C. and 45° C. in a shaker-incubator for 1 hour at 70 RPM, the cap was left loose to allow oxygen, ammonia, and carbon dioxide gases to be released. Ammonium hydroxide and ammonium bicarbonate were removed, and thus, the solution neutralized by evaporation. Sodium hydroxide was neutralized by the addition of HCl to pH 7. The sample was stored at −20° C. prior to clean-up and subsequent mass spectrometry analysis.

Isolation of Oligosaccharides. Post-cleavage oligosaccharide samples were reconstituted in water and subjected to C18 solid phase extraction. Solid phase cartridges were washed with three volumes acetonitrile and two volumes water before samples were loaded and collected as the immediate flow-through. The C18 cartridge-extracted samples were then subjected to non-porous graphitized carbon (NPGC) solid phase extraction. NPGC cartridges were sequentially pre-washed with two volumes water, two volumes of 80% acetonitrile with 0.01% (v/v) TFA in water, and two more volumes of water. The C18 cartridge-extracted samples were then loaded and washed with five volumes of water before being eluted with 40% acetonitrile with 0.05% (v/v) TFA. Finally, the post-NPGC samples were completely dried by evaporative centrifugation and stored at −20° C. until analysis.

Instrumental Analysis. Dried, post-NPGC samples were reconstituted in nano-pure water before UHPLC-QqQ analysis. Analytical separation was performed using an Agilent 1290 Infinity II UHPLC coupled to an Agilent 6495 QqQ MS. Samples were chromatographically separated on a 50 mm×1 mm Waters Acquity™ BEH-AMIDE column with a 1.9 μm particle size. A binary gradient was employed which consisted of solvent A: (3% (v/v) acetonitrile/water+0.1% formic acid) and solvent B: (95% acetonitrile/water). A 4.5-minute gradient with a flow rate of 0.6 ml/min was used for chromatographic separation: 70-67% B, 0-3 min; 67-25% B, 3-3.01 min; 25-25% B, 3.01-3.5 min; 25-70% B, 3.5-3.51 min; 70-70% B, 3.51-4.5 min. Electrospray ionization was used as the ion source and data was collected in the positive mode and utilized single ion monitoring (SIM). The capillary and fragmentor voltage were 1800 and 280 V, respectively. The quadrapole was set to scan masses corresponding to oligosaccharides from 2-10 hexoses with a dwell time of 50 ms. All ions were observed as their proton adduct.

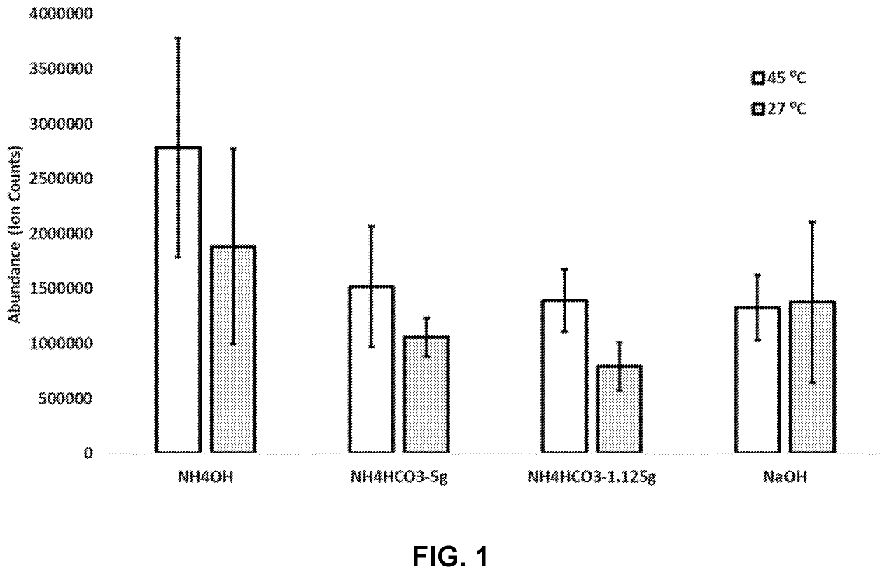

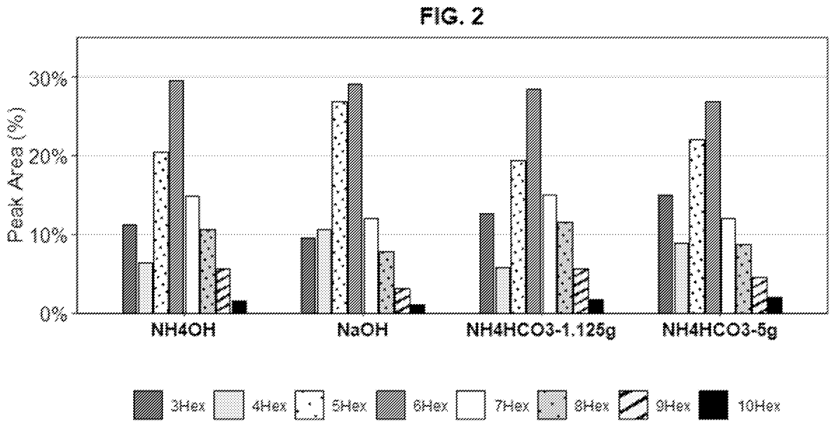

Ammonium hydroxide and Ammonium Bicarbonate as exemplary PS-cleavage Reagents. Of the three cleavage reagents, ammonium hydroxide produced the highest amount of total oligosaccharides from locust bean gum at both 45° C. and 27° C., followed by both ammonium bicarbonate concentrations at 45° C., sodium hydroxide at both temperatures, and lastly, ammonium bicarbonate at 27° C. (FIG. 1). Unexpectedly, ammonium hydroxide produced highest overall abundance of oligosaccharides without sacrificing oligosaccharide structural diversity (FIG. 2), and produced two-fold more total oligosaccharides compared to sodium hydroxide and ammonium bicarbonate. The results were further unexpected, since it was expected that the concentration of hydroxide ions that are readily present in ammonium hydroxide and sodium hydroxide would be correlated with the production of oligosaccharides in the PS-cleavage step of the COG reactions; however, both the ammonium hydroxide and sodium hydroxide reactions reached pH 10, which indicated that the same concentration of hydroxide ions. Additionally, it was unexpected that ammonium bicarbonate would produce a substantial amount of oligosaccharides considering that at 27° C. the pH was only 7.5, although it nearly matched the oligosaccharide production of NaOH as the cleavage reagent. According to particular aspects, these results indicate that a non-hydroxide mediated mechanism is mediating the cleavage by ammonium bicarbonate. Furthermore, the enhanced efficiency of ammonium hydroxide as a PS-cleavage reagent may indicate that a combination of both hydroxide-driven and ammonia-driven mechanisms is involved.

Mechanistic differences are also observed in the specificity of the reaction in regard to the size distribution of the oligosaccharides produced (FIG. 2). Oligosaccharides from 3-10 monomers in length were observed from the 45° C. reactions. Notable differences in the relative concentration of trisaccharides were observed. The two ammonium bicarbonate-mediated PS-cleavage reactions produced the highest relative abundance of trisaccharides (15.0% and 12.7%), while the sodium hydroxide-mediated PS-cleavage reaction produced the least (9.52%), with ammonium hydroxide-mediated PS cleavage being in the middle (11.2%). The differential production of trisaccharides between the sodium hydroxide-mediated and the ammonium bicarbonate-mediated PS-cleavage reactions provides additional evidence of a different reaction mechanism, and the intermediate amount of trisaccharide production mediated by ammonium hydroxide yet further supports a combinatorial PS-cleavage mechanism. Other notable differences include sodium hydroxide producing the largest relative abundances of the tetrasaccharide and pentasaccharide, while the hexasaccharide remained the most abundant oligosaccharide in all four conditions. Furthermore, all three of the nitrogen-based reagent-mediated cleavage reactions produced higher amounts of the decasaccharide than did sodium hydroxide.

Without being bound by mechanism, applicant's data is consistent with a mechanism wherein cleaving hydroperoxyl radical-treated polysaccharide with, e.g., nitrogen-based cleavage agents (instead of with the strong Arrhenius bases used in the art) proceeds by a unique $-elimination mechanism involving deprotonation by, e.g., ammonia (or a decomposition product of the cleavage agent, e.g., of the nitrogen-based cleavage agents) of a hydroxyl moiety positioned $ relative to a glycosidic bond, due to an adjacent ketone (from the peroxidation step) that pulls electron density away from the hydrogen, thereby rendering it a better leaving group. As proposed, when the e.g., ammonia deprotonates the carbohydrate, the electrons form a carbon-carbon double bond that facilitates breaking of the glycosidic bond, thus depolymerizing the polysaccharide.

### Example 2

(The Need for Desalting was Reduced or Eliminated when Ammonium Bicarbonate or Ammonium Hydroxide was Used as a Polysaccharide (PS)-Cleavage Reagent)

Desalting oligosaccharides is an expensive and time-consuming process that, prior to the present disclosure, represented a major limitation in the production of oligosaccharides using prior Fenton's reagent-based methods. Dialysis and chromatographic desalting are two common processes for separating oligosaccharides from the salts (e.g., sodium chloride, sodium acetate, and potassium chloride) produced upon neutralization of the traditional strong Arrhenius base (e.g., NaOH, KOH, Ca(OH)₂) used in generating the oligosaccharides. Both processes prove difficult as low molecular weight salts such as sodium chloride (58.44 g/mol) and oligosaccharides such as maltotriose (504.44 g/mol) are close enough in mass to make separation difficult. Both processes additionally require that the sample first be reduced in volume prior to separation, which further increases both the cost and required process time. According to particular aspects, the presently disclosed use of high-yield nitrogen-based peroxide-quenching/PS-cleavage agents (e.g., ammonium bicarbonate, ammonium hydroxide, ammonia, etc.) eliminates the need for desalting via dialysis or other size-based methods because such agents, or the reactions products thereof can be evaporated from solution upon reaction completion. The ammonium bicarbonate, for example, can be efficiently removed as CO₂, NH₃, and H₂O according to the reaction mechanism:

NH₄⁺HCO₃⁻(g)⇔NH₃(g)+CO₂(g)+H₂O(l)

while ammonium hydroxide, for example, can be removed as NH₃, and H₂O according to the reaction mechanism:

NH₄⁺OH⁻(s)⇔NH₃(g)+H₂O(l).

### Example 3

(Ammonium-Based PS-Cleavage Reagents were Shown to be Peroxide-Quenching Reagents that Eliminated Hydrogen Peroxide and Off-Target Oxidation, and Thus Represent Exemplary, Preferred Peroxide-Quenching/PS-Cleavage Reagents)

While hydrogen peroxide is a component of the initial oxidative step in production of oligosaccharides as disclosed herein (and in prior art methods), there is a danger of unwanted, off-target oxidation from any residual presence of hydrogen peroxide and/or of its radicals in the subsequent cleavage reaction step, and any subsequent downstream processing steps. As appreciated in the art, due to its high boiling point (150.2° C.), hydrogen peroxide cannot be easily removed through standard evaporative processes. Furthermore, its presence can hinder chromatographic efforts for downstream glycan purification and enrichment as many stationary phases are not stable against high red/ox states. Strategies for its removal can include dialysis, the use of enzymes such as horseradish peroxidase, and prolonged exposure to an open atmosphere environment. Enzymatic methods have the advantage of quenching the hydrogen peroxide quickly but will also require removal down-stream. Both dialysis and exposure to open atmosphere environments leave the hydrogen peroxide (and/or any residual radicals thereof) in contact with the produced oligosaccharides, which can produce side reactions including C-6 oxidation to create -uronic acid containing oligosaccharides and other unwanted species. According to particular aspects, the presently-disclosed COG methods solve this substantial problem.

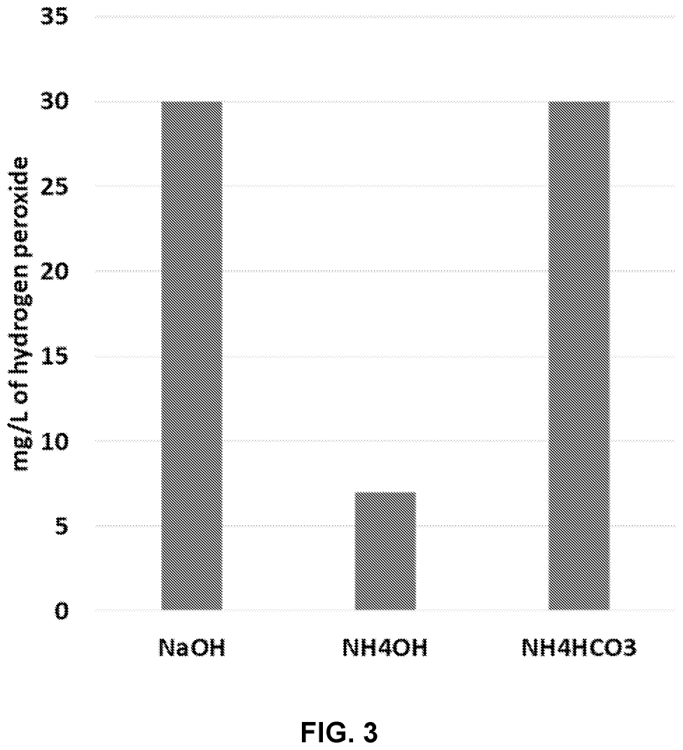

To determine the effects of different PS-cleavage reagents on the concentration of hydrogen peroxide, hydrogen peroxide concentrations were measured with test strips (Quantofix Peroxide 100™) after incubation with different cleaving reagents and temperatures. Three PS-cleavage reagents, ammonium hydroxide, ammonium bicarbonate, and sodium hydroxide were first incubated with locust bean gum treated with hydrogen peroxide and Fe(JJ). The use of ammonium hydroxide at room temperature was shown to quickly eliminate the presence of hydrogen peroxide. By contrast, however, neither ammonium bicarbonate nor sodium hydroxide had an effect on the concentration of hydrogen peroxide (FIG. 3). This confirms that while prior Fenton's-based methods used a strong Arrhenius base to quench the Fenton's reaction, there was no quenching or elimination of the residual hydrogen peroxide per se, or of any residual radicals thereof produced before introduction of the strong Arrhenius base.

According to particular aspects, the following mechanisms:

2NH₃(g)+H₂O₂(l)⇔N₂(g)+2H₂O(l)+2H₂(g)

2NO₂⁻2H⁺aq⇔H₂O+NO+NO₂

support applicant's conception that as ammonia is produced (or otherwise introduced into the reaction), some residual hydrogen peroxide, or radicals thereof will be quenched/eliminated.

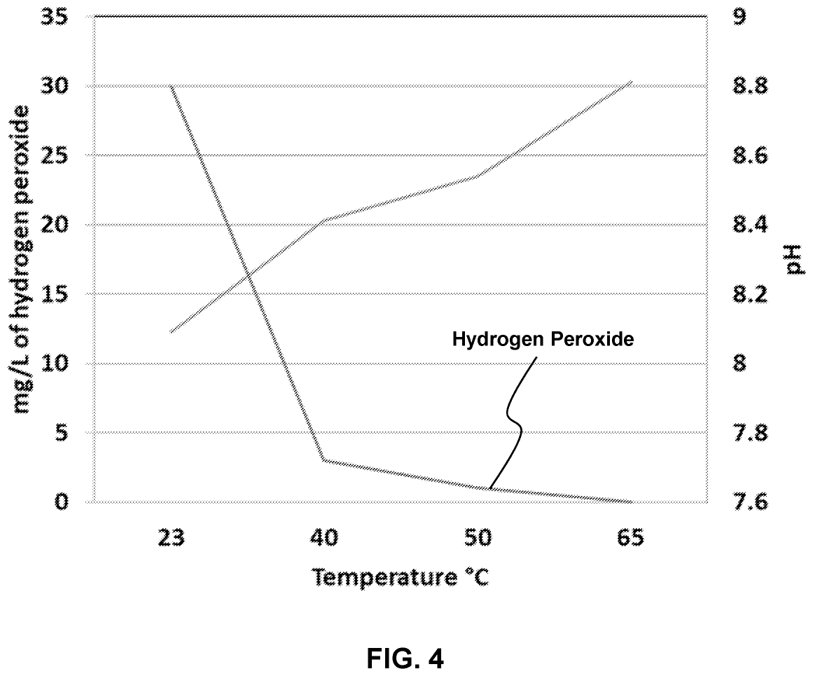

To further test this proposed mechanism, the reactions with the three cleaving reagents were heated for one hour at increasingly higher temperatures, up to 65° C. to drive the ammonium bicarbonate solution to produce more ammonia. Indeed, the reaction pH increased with increasing temperature, indicating the presence of hydroxide ions, which would be accompanied by ammonia gas, and thus the simultaneous quenching of hydrogen peroxide was observed (FIG. 4). Moreover, this reaction happened rapidly at 40° C. and ultimately resulted in hydrogen peroxide levels that were below detectable limits at 65° C. Significantly, there was no observable difference in the hydrogen peroxide concentration in the heated sodium hydroxide solution. The data confirms that hydrogen peroxide is rapidly quenched/eliminated when incubated, for example, with the presently-disclosed nitrogen-based cleavage reagents, but not with traditional strong Arrhenius bases (e.g., Na⁺OH⁻, K⁺OH⁻, or Ca²⁺(OH⁻)₂) previously used in the art.

### Example 4

(Ammonium Hydroxide Produced Unique Oligosaccharide Profiles from Spent Grain Fractions)

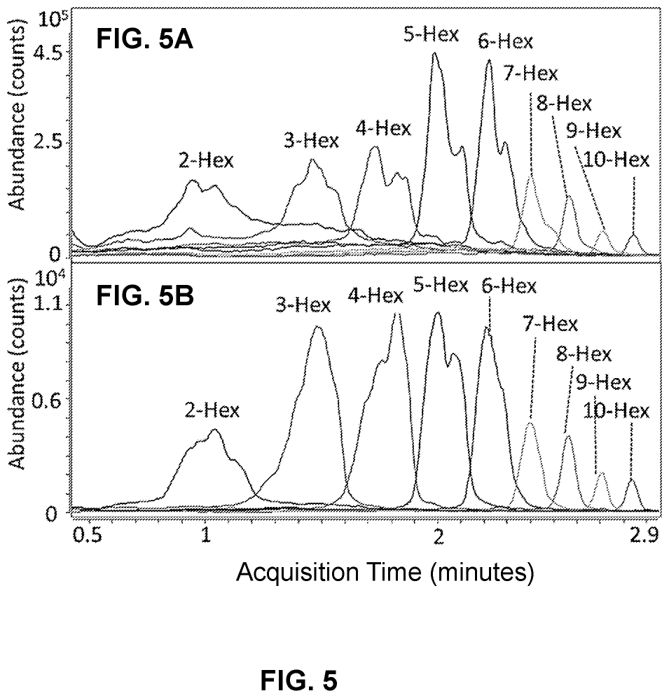

Two spent grain fractions were ground to a fine powder and underwent the procedure described in Example 1, while employing ammonium hydroxide as a peroxide-quenching/PS-cleavage reagent. The grain samples represented a “whole” spent fraction and a protein-depleted fraction, produced and recovered from a bio-ethanol production process. A liquid chromatography-mass spectrum obtained from the depolymerization products of the two fractions (FIGS. 5A and 5B) showed that both fractions included abundant hexose oligomers that ranged from 3-10 monomeric units; however, larger structures are also abundant but were not monitored under the presented conditions. The protein-depleted fraction (FIG. 5B) contained both a higher concentration of total carbohydrate and produced roughly twice the concentration of oligosaccharides than the “whole” spent grain product (FIG. 5A). Non-carbohydrate components of complex mixtures can inhibit the effects of the reaction by competing for the oxidative and cleaving potential of the reactions (Stadtman and Berlett 1991). This result nonetheless demonstrates the broad effectiveness of the disclosed CDPG methods (e.g., involving use of non-Arrhenius and weak-Arrhenius bases as peroxide-quenching/PS-cleavage reagents after prior Fenton oxidation.

### Example 5

(In the Disclosed COG Reactions, Peroxide-Quenching May be Initiated Prior to, Commensurate with, or Subsequent to Initiation of Polysaccharide (PS)-Cleavage)

Exemplary PS-cleavage, and/or peroxide-quenching agents are listed in Table 1 above.

In preferred COG method aspects, as disclosed and discussed above herein, the COG methods overcome a substantial problem in the art by using a hydrogen peroxide quenching agent (“peroxide-quenching” agent) to reduce or eliminate off-target side reactions after initiation of the PS-cleavage step. While prior methods are use strong-Arrhenius bases (i.e., Na⁺OH⁻, K⁺OH⁻, or Ca²⁺(OH⁻)₂) as PS-cleavage agents to allegedly “quench” the initial Fenton's reaction (i.e., by flocculating the metal ion reactant), such strong-Arrhenius base PS-cleavage agents do not (as disclosed herein; e.g., see Example 3, above) quench/eliminate residual peroxide or peroxide radicals per se, and thus prior art methods are susceptible to unwanted side reactions.

In preferred COG methods, the PS-cleavage initiator preferably also functions as a peroxide-quencher to quench (e.g., sufficiently reduce or eliminate) residual hydrogen peroxide and/or radicals thereof per se, to minimize or eliminate off-target side reactions. In such method aspects, initiation of peroxide-quenching (and thus also quenching of the Fenton's reaction) is commensurate with initiation of PS-cleavage. While such COG reactions may simplistically be viewed as two-step reaction aspects (comprising a Fenton's oxidation aspect followed by a PS-cleavage aspect), it is to be understood that peroxide-quenching (and/or quenching of the Fenton's reaction) may or may not be immediate or sharply delineated, and may yet occur over at least part of the PS-cleavage aspect; that is, despite the use of peroxide-quenching agents as disclosed herein, there may be at least some degree of overlap between the Fenton's reaction aspect, the peroxide-quenching aspect, and/or the PS-cleavage aspect of such COG reactions. The degree of overlap may vary depending on the nature and amount of the peroxide-quenching agent used.

In particular COG methods, the PS-cleavage agent (cleavage initiator) may or may not also be a peroxide-quenching agent, and in either case may be used in combination with an additional compatible peroxide-quenching agent, which itself may or may not also be a cleavage agent. In such aspects, the additional compatible peroxide-quenching agent may be introduced into the reaction prior to, commensurate with, or subsequent to introduction of the PS-cleavage agent.

In particular aspects the additional compatible peroxide-quenching agent is introduced into the reaction commensurate with introduction of the PS-cleavage agent. While such COG reaction aspects may simplistically be viewed as two-step reaction aspects (comprising a Fenton's oxidation aspect followed by a PS-cleavage aspect) it is to be understood that peroxide-quenching (and/or quenching of the Fenton's reaction) may or may not be immediate or sharply delineated, and may yet occur over at least part of the PS-cleavage aspect; that is, despite the use of peroxide-quenching agents as disclosed herein, there may be at least some degree of overlap between the Fenton's reaction aspect, the peroxide-quenching aspect, and/or the PS-cleavage aspect of such COG reactions. The degree of overlap may vary depending on the nature and amount of the peroxide-quenching agent used.

In particular aspects the additional compatible peroxide-quenching agent is introduced into the reaction prior to introduction of the PS-cleavage agent. While such COG reaction aspects may simplistically be viewed as two-step reaction aspects (comprising a Fenton's oxidation aspect followed by a PS-cleavage aspect), or as three-step reaction aspects (comprising a Fenton's oxidation aspect, followed by a peroxide-quenching aspect, followed by a PS-cleavage aspect), it is to be understood that peroxide-quenching (and/or quenching of the Fenton's reaction) may or may not be immediate or sharply delineated, and may yet occur over at least part of the PS-cleavage aspect; that is, despite the use of peroxide-quenching agents as disclosed herein, there may be at least some degree of overlap between the Fenton's reaction aspect, the peroxide-quenching aspect, and/or the PS-cleavage aspect of such COG reactions. The degree of overlap may vary depending on the nature and amount of the peroxide-quenching agent used.

In particular aspects the additional compatible peroxide-quenching agent is introduced into the reaction subsequent to introduction of the PS-cleavage agent. While such COG reaction aspects may simplistically be viewed as two-step reaction aspects (comprising a Fenton's oxidation aspect followed by a PS-cleavage aspect), or as three-step reaction aspects (comprising a Fenton's oxidation aspect, followed by a PS-cleavage aspect, followed by a peroxide-quenching aspect), it is to be understood that peroxide-quenching (and/or quenching of the Fenton's reaction) may or may not be immediate or sharply delineated, and may yet occur over at least part of the PS-cleavage aspect; that is, despite the use of peroxide-quenching agents as disclosed herein, there may be at least some degree of overlap between the Fenton's reaction aspect, the peroxide-quenching aspect, and/or the PS-cleavage aspect of such COG reactions. The degree of overlap may vary depending on the nature and amount of the peroxide-quenching agent used.

According to some aspects, in all of the above COG method aspects, use of a peroxide-quencher to quench (e.g., sufficiently reduce or eliminate) residual hydrogen peroxide and/or radicals thereof per se, minimizes or eliminates off-target side reactions.

According to some aspects, in all of the above COG method aspects, use of particular weak Arrhenius bases and/or non-Arrhenius bases (e.g., nitrogen-based peroxide-quenching/PS-cleavage reagents, etc.; e.g., see Table 1) not only provides for improved high-yield oligosaccharide production (relative to the strong Arrhenius bases used in the art), but also eliminates the need for costly and time-consuming post-reaction concentration, and desalting steps.

### Example 6

(COG Offers Enhanced Bioactivity when Compared to Similar Methods)

In some aspects oligosaccharides generated from COG can be used to promote the growth of bacteria in fermentations (biotechnology, ethanol production, food processing) and/or the microbiota of humans and animals (gut, skin, respiratory, vaginal, ocular, oral). Common methods of assessing the ability for microbes to consume particular oligosaccharides and groups of oligosaccharides entail their monitoring by optical density across the growth period. However, if the oligosaccharides are contaminated by endogenous or exogenous materials, these results can be erroneous. Compounds such as salts, acids, metals, and oxidizing/reducing agents can inhibit bacterial growth in in vitro systems.

Oligosaccharide production: In this exemplary aspect, amylopectin (550 mg) was dissolved in 20 ml of HPLC grade water in a capped reaction vessel and placed in a shaker-incubator for 20 min at 55° C. and 85 RPM. The pH of the solution was adjusted to 5.2. Hydrogen peroxide (5 ml) and iron (II) sulfate (2.75 mg in 50 μL water) were added to the reaction mixture and mixed thoroughly. The reaction in the capped reaction vessel proceeded in the shaker-incubator at 55° C. and 65 RPM for two hours. The capped reaction cooled to 12° C. in a −20° C. freezer. Ammonium hydroxide (1 ml of 28% v/v to pH 10.2) or NaOH (600 ul of 10.45 M) was used to adjust pH and sample was reacted at 45° C. in a shaker-incubator for 1 hour at 20 RPM, the cap was left loose to allow oxygen, ammonia, and carbon dioxide gases to be released. The sample was then frozen and lyophilized, then stored at −80° C. Size exclusion chromatography was conducted on a 50 mL Bio-Scale™ Mini Bio-Gel® P-6 Desalting Cartridge using 0.03M ammonium bicarbonate buffer at a flow rate of 10 mL/min. For the purpose of desalting, an elution window of 50 mL was collected post void volume and the samples were lyophilized to complete dryness. The resulting materials were analyzed for concentrations of iron, hydrogen peroxide, and sulfate, pH, oxidative/reductive potential (ORP), and electrical conductivity (EC).

Carbohydrate analysis: Post-cleavage oligosaccharide samples were reconstituted in water and subjected to alditol reduction. Samples were reduced for 1 hour with 2 M sodium borohydride at 65° C., then immediately underwent C18 solid phase extraction. Solid phase cartridges were washed with three volumes acetonitrile and two volumes water before samples were loaded and collected as the immediate flow-through. The C18 cartridge-extracted samples were then subjected to non-porous graphitized carbon (NPGC) solid phase extraction. NPGC cartridges were sequentially pre-washed with two volumes water, two volumes of 80% acetonitrile with 0.01% (v/v) TFA in water, and two more volumes of water. The C18 cartridge-extracted samples were then loaded and washed with five volumes of water before being eluted with 40% acetonitrile with 0.05% (v/v) TFA. Finally, the post-NPGC samples were completely dried by evaporative centrifugation and stored at −20° C. until analysis.

Oligosaccharide analysis was carried out on an Agilent 1290 Infinity II HPLC coupled to an Agilent 6530 Accurate-Mass Q-TOF MS. Chromatographic separation was performed on a Thermo Scientific Hypercarb PGC column with a binary gradient which consisted of solvent A: 3% acetonitrile/water+0.1% formic acid and solvent B: 10% water/acetonitrile+0.1% formic acid. With a flow rate of 0.15 mL/min, the gradient was run for 60 min: 2-15% B, 0-20 min; 15-60% B, 20-45 min; 60-99% B, 45-45.10 min; 99-99% B, 45.10-51 min; 99-2% B, 51-51.10 min; 2-2% B, 51.10-60 min. The mass spectrometer was run in positive mode, with a reference mass of 922.0098 m/z. The gas temperature and flow rate were set to 150° C. and 11 l/min, respectively. The nozzle, fragmentor, skimmer voltages were set to be 1500, 75 and 60 volts, respectively. Using tandem mass spectrometry, fragmentation was performed with collision energy of 1.45×(m/z)−3.5. Data was processed using Agilent MassHunter Workstation Quantitative Analysis 10.1 Software. Major peaks in the chromatograms that corresponded to oligosaccharide masses were integrated. Responses of oligosaccharides with DP 2-10 were summed to represent the total oligosaccharide peak area.

Furthermore, the samples were analyzed for their monosaccharide composition as described in Amicucci et al (Amicucci, Galermo, et al. 2019).

Bacterial Growth Method: Ability of the generated oligosaccharide fractions to support bacterial growth was evaluated by incubating a Bifidobacterium breve (model organism) in minimal media supplemented with 3% (m/v) of oligosaccharide fraction at 37 C under anaerobic conditions. Minimal media used for these experiments was basal MRS (Ruiz-Moyano et al. 2013). Before inoculation basal MRS was mixed with lactose and each of the oligosaccharide fractions, pH was adjusted to 6.8, filter sterilized and placed in the anaerobic chamber for approximately 12 hours to remove oxygen. Triplicates of each treatment, including a positive (1% lactose only) and a negative control (no carbohydrate) were inoculated with 2% of a fresh culture of Bifodobacterium and incubated under anaerobic conditions. Growth was determined based on absorbance measurements at 600 nm for 24 hours. Media sterility was tested by incubating non-inoculated media.

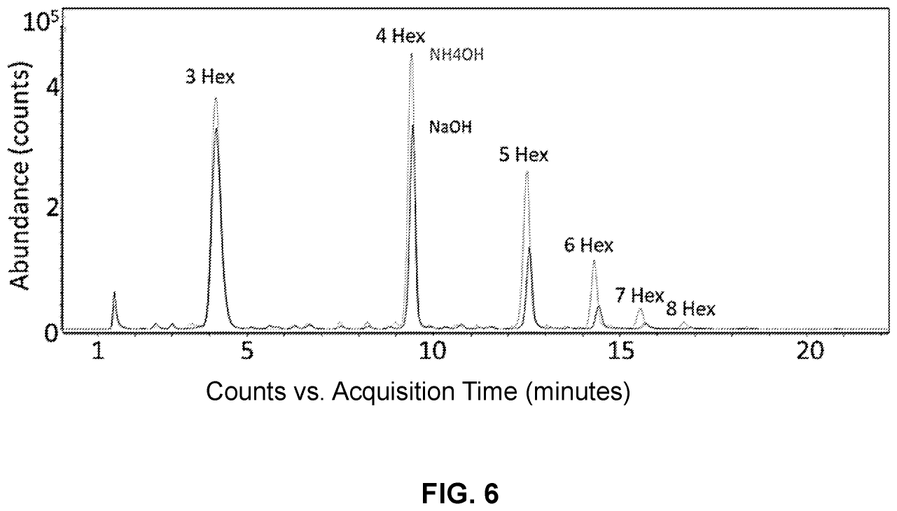

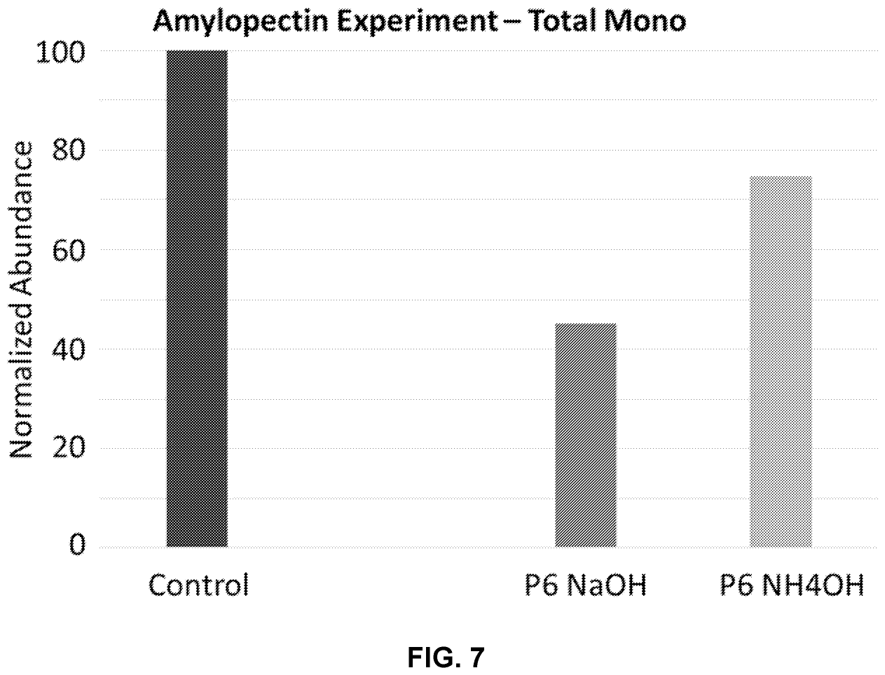

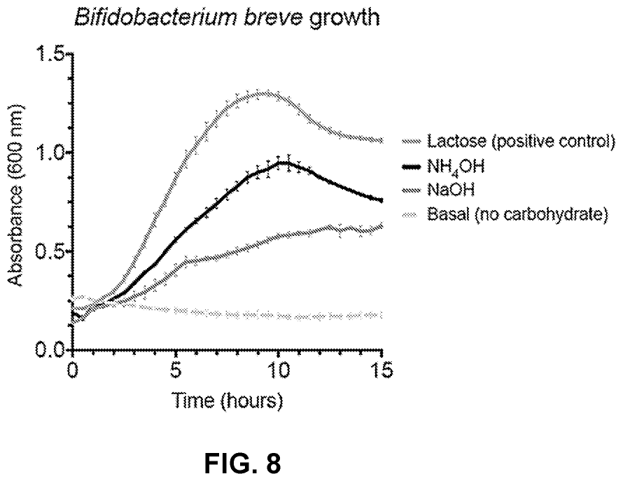

Results: Oligosaccharides produced by ammonium hydroxide cleavage produced a higher yield of oligosaccharides than the sodium hydroxide (FIG. 6) and also a purer final product as indicated by quantitative monosaccharide analysis (FIG. 7). Furthermore, the ammonium hydroxide showed lower ORP, indicating less residual hydrogen peroxide, and similar conductivity (EC), indicating lower ionic content (Table 2). Oligosaccharides generated using NH₄OH showed a stronger growth response than their counterparts generated with NaOH (FIG. 8). While NH₄OH oligosaccharides supported bifidobacteria growth to a max OD (620 nm) of 0.974, cell density with NaOH oligosaccharides only reached a max OD (620 nm) of 0.64. This demonstrates bifidobacteria substrate preference of NH₄—H over NaOH oligosaccharides and suggests that this and other COG fractions will enrich other bacterial groups in a similarly superior manner,

TABLE 2
Physical properties of oligosaccharide pools.

| Post COG with NaOH | Post COG with NH4OH |
|---|---|
| pH: 7.62 | pH: 5.69 |
| EC: 4.31 μS/cm] | EC: 7.40 μS/cm] |
| ORP: −378 mV | ORP: −132.9 mV |
| Fe2/Fe3: 5 mg/L | Fe2/Fe3: 5 mg/L |
| Peroxide: 0 mg/L | Peroxide: 0 mg/L |
| Sulfate: <200 mg/L | Sulfate: >400 mg/L |

### Example 7

### (Oligosaccharide Profiles are Dependent on Base Selection)

In an exemplary aspect, Amylopectin (200 mg) was dissolved in 7 ml of HPLC grade water in a capped reaction vessel and placed in a shaker-incubator for 20 min at 55° C. and 85 RPM. The pH of the solution was adjusted to 5.2. Hydrogen peroxide (1.75 ml) and iron (II) sulfate (1 mg in 25 μL water) were added to the reaction mixture and mixed thoroughly. The reaction in the capped reaction vessel proceeded in the shaker-incubator at 55° C. and 65 RPM for two hours. The capped reactions cooled to 12° C. in a −20° C. freezer. Seven bases were examined: Pyridine (100 ul), N, N-Diisopropylethylamine (DIPEA), NaOH (35 μl, 10 M), CsOH (35 μl, 10 M), Ca(OH)₂ (45 μl, 10 M), KOH (35 μl, 10 M) and NH₄OH (500 μl of 28% v/v). All bases were added to the reaction mixture to a pH of 10, except pyridine which reached pH 9. All seven conditions were reacted at 45° C. in a shaker-incubator for 1 hour at 20 RPM, the cap was left loose to allow oxygen, ammonia, and carbon dioxide gases to be released. The samples were frozen and lyophilized than stored at −80° C., prior to clean-up and subsequent mass spectrometry analysis.

Carbohydrate analysis: Post-cleavage oligosaccharide samples were reconstituted in water and subjected to alditol reduction. Samples were reduced for 1 hour with 2 M sodium borohydride at 65° C., then immediately underwent C18 solid phase extraction. Solid phase cartridges were washed with three volumes acetonitrile and two volumes water before samples were loaded and collected as the immediate flow-through. The C18 cartridge-extracted samples were then subjected to non-porous graphitized carbon (NPGC) solid phase extraction. NPGC cartridges were sequentially pre-washed with two volumes water, two volumes of 80% acetonitrile with 0.01% (v/v) TFA in water, and two more volumes of water. The C18 cartridge-extracted samples were then loaded and washed with five volumes of water before being eluted with 40% acetonitrile with 0.05% (v/v) TFA. Finally, the post-NPGC samples were completely dried by evaporative centrifugation and stored at −20° C. until analysis.

(Amicucci, Galermo et al. 2019). Oligosaccharide analysis was carried out on an Agilent 1290 Infinity II HPLC coupled to an Agilent 6530 Accurate-Mass Q-TOF MS. Chromatographic separation was performed on a Thermo Scientific Hypercarb PGC column with a binary gradient which consisted of solvent A: 3% acetonitrile/water+0.1% formic acid and solvent B: 10% water/acetonitrile+0.1% formic acid. With a flow rate of 0.15 mL/min, the gradient was run for 60 min: 2-15% B, 0-20 min; 15-60% B, 20-45 min; 60-99% B, 45-45.10 min; 99-99% B, 45.10-51 min; 99-2% B, 51-51.10 min; 2-2% B, 51.10-60 min. The mass spectrometer was run in positive mode, with a reference mass of 922.0098 m/z. The gas temperature and flow rate were set to 150° C. and 11 1/min, respectively. The nozzle, fragmentor, skimmer voltages were set to be 1500, 75 and 60 volts, respectively. Using tandem mass spectrometry, fragmentation was performed with collision energy of 1.45×(m/z)−3.5. Data was processed using Agilent MassHunter Workstation Quantitative Analysis 10.1 Software. Major peaks in the chromatograms that corresponded to oligosaccharide masses were integrated. Responses of oligosaccharides with DP 2-10 were summed to represent the total oligosaccharide peak area.

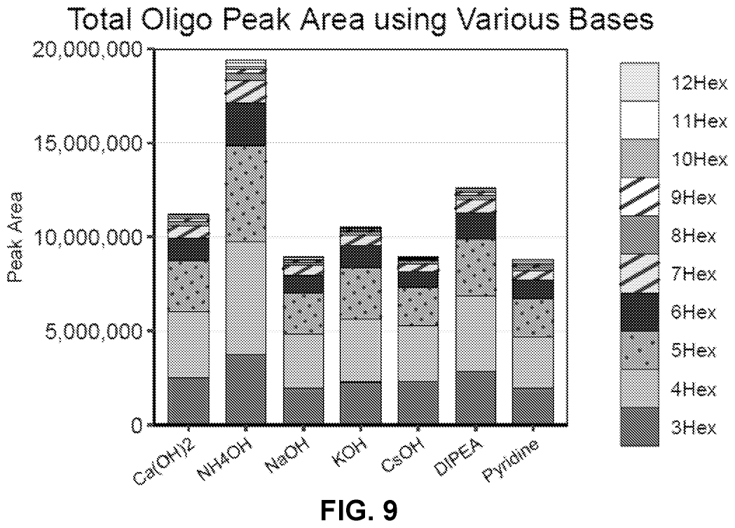

The oligosaccharide analysis showed different amounts of oligosaccharide production on both the total and structure specific level. All of the bases used in this experiment did produce oligosaccharide products. Ammonium hydroxide produced the highest concentration of oligosaccharides, nearly double the sodium hydroxide (FIG. 9). Additionally, DIPEA, a nitrogen containing base, produced the second largest concentration of oligosaccharides, while pyridine produced oligosaccharides on-par with the Arrhenius bases. This provides further evidence for the broad classification of “nitrogen-based cleavage reagents” to be considered good cleavage reagents.

### Example 8

### (Iron (II) and the Production of Locust Bean Oligosaccharides)

In some aspects the oxidation state of the metal can be changed for similar or different results. In this described aspect, Iron (II) was used to produce oligosaccharides from locust bean gum polysaccharide. Locust bean gum contains a galactomannan polymer that contains a β1,4 mannose backbone with terminal branches of a 1,6 galactose.

Oligosaccharide production: Locust bean gum (550 mg) was dissolved in 20 ml of HPLC grade water in a capped reaction vessel and placed in a shaker-incubator for 20 min at 55° C. and 85 RPM. The pH of the solution was adjusted to 5.2. Hydrogen peroxide (5 ml) and iron (II) sulfate (2.75 mg in 50 μL water) were added to the reaction mixture and mixed thoroughly. The reaction in the capped reaction vessel proceeded in the shaker-incubator at 55° C. and 65 RPM for two hours. The capped reaction cooled to 12° C. in a −20° C. freezer. Ammonium hydroxide (1 ml of 28% v/v to pH 10.2) was used to adjust pH and sample was reacted at 45° C. in a shaker-incubator for 1 hour at 20 RPM, the cap was left loose to allow oxygen, ammonia, and carbon dioxide gases to be released. The sample is then frozen and lyophilized, then stored at −80° C. The freeze-dried oligosaccharide mixture was rehydrated with the minimum amount of water required to allow for a free-flowing solution. This solution was then loaded onto a column containing 15 mL mixed bed ion exchange resin per gram (dry weight) of crude material, and the runoff was collected in a plastic freezer bag. Once the material was loaded onto the column, the column was then rinsed with 3 bed volumes of water. Finally, the runoff was sealed and frozen in the bag, then carefully shattered and subjected to lyophilization.

Carbohydrate analysis: Post-cleavage oligosaccharide samples were reconstituted in water and subjected to alditol reduction. Samples were reduced for 1 hour with 2M sodium borohydride at 65° C., then immediately underwent C18 solid phase extraction. Solid phase cartridges were washed with three volumes acetonitrile and two volumes water before samples were loaded and collected as the immediate flow-through. The C18 cartridge-extracted samples were then subjected to non-porous graphitized carbon (NPGC) solid phase extraction. NPGC cartridges were sequentially pre-washed with two volumes water, two volumes of 80% acetonitrile with 0.01% (v/v) TFA in water, and two more volumes of water. The C18 cartridge-extracted samples were then loaded and washed with five volumes of water before being eluted with 40% acetonitrile with 0.05% (v/v) TFA. Finally, the post-NPGC samples were completely dried by evaporative centrifugation and stored at −20° C. until analysis.

(Amicucci, Galermo et al. 2019). Oligosaccharide analysis was carried out on an Agilent 1290 Infinity II HPLC coupled to an Agilent 6530 Accurate-Mass Q-TOF MS. Chromatographic separation was performed on a Thermo Scientific Hypercarb PGC column with a binary gradient which consisted of solvent A: 3% acetonitrile/water+0.1% formic acid and solvent B: 10% water/acetonitrile+0.1% formic acid. With a flow rate of 0.15 mL/min, the gradient was run for 60 min: 2-15% B, 0-20 min; 15-60% B, 20-45 min; 60-99% B, 45-45.10 min; 99-99% B, 45.10-51 min; 99-2% B, 51-51.10 min; 2-2% B, 51.10-60 min. The mass spectrometer was run in positive mode, with a reference mass of 922.0098 m/z. The gas temperature and flow rate were set to 150° C. and 11 1/min, respectively. The nozzle, fragmentor, skimmer voltages were set to be 1500, 75 and 60 volts, respectively. Using tandem mass spectrometry, fragmentation was performed with collision energy of 1.45×(m/z)−3.5. Data was processed using Agilent MassHunter Workstation Quantitative Analysis 10.1 Software. Major peaks in the chromatograms that corresponded to oligosaccharide masses were integrated. Responses of oligosaccharides with DP 2-10 were summed to represent the total oligosaccharide peak area.

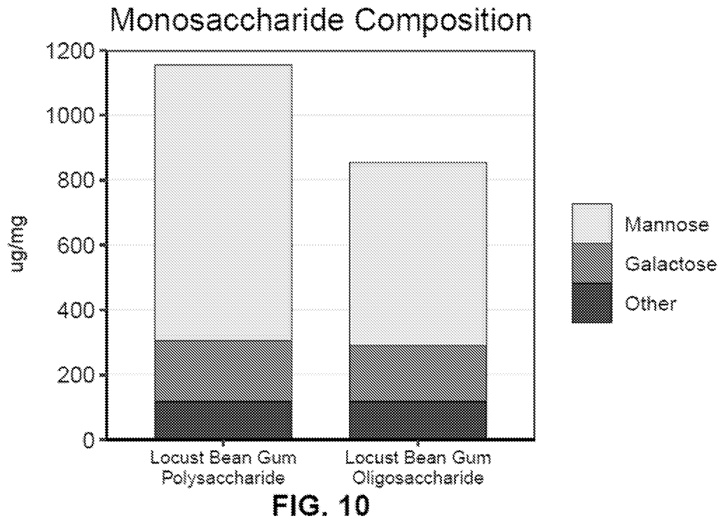

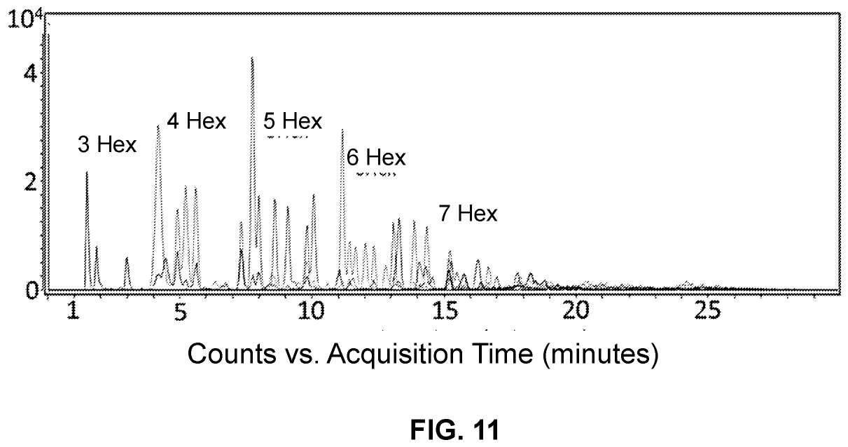

Results: The oligosaccharides produced from the Fe(II) oxidation and cleavage of locust bean gum produced oligosaccharides that resembled their parent locust bean polysaccharide structure, besides for their degree of polymerization, which were much shorter. The monosaccharide analysis indicated a high level of purity (>90%) and a similar monomeric composition as the parent polymer, 3.17:1 vs. 4.52:1 mannose:galactose, respectively (FIG. 10). The oligosaccharide analysis revealed oligosaccharides from 3 to 8 hexoses in length that that contain a plethora of isomers (FIG. 11). Locust bean gum is known to contain the galactomannan polysaccharide which contains a β1,4 mannose backbone single α 1,6 linked galactoses branching from the backbone.

### Example 9

(Arabinoxylan Oligosaccharides Derived from Corn Fiber.)

Corn fiber is a highly abundant waste stream from the leftover fermentation of corn to produce ethanol. This material comprises several abundant polysaccharides including, beta-glucan, arabinoxylan, cellulose, and residual amylose and amylopectin. The arabinoxylan components offer an opportunity for producing arabinoxylan oligosaccharides, which have been shown to modulate the gut microbiome (Neyrinck et al. 2012).

Corn fiber was subjected to purification via a chloroform extraction where 5 g of the material was suspended in 100 ml of chloroform and allowed to mix for approximately 2 hours. The resulting mixture was then crashed with 50 mL of 0° C. water, producing a viscous material. The mixture was centrifuged for 30 min at 6500 rpm discarding the liquid layer. The bottom layer was then resuspended in 10 ml of water and crashed with absolute ethanol at 0° C. An additional two subsequent washes with absolute ethanol at 0° C. were conducted to produce a white polysaccharide precipitate. Material was subjected to drying by lyophilization, producing 4.8 g.

The material was subjected to the COG reaction under the following conditions. 550 mg was dissolved in 20 ml of HPLC grade water in a capped reaction vessel and placed in a shaker-incubator for 20 min at 55° C. and 85 RPM. The pH of the solution was adjusted to 5.2. Hydrogen peroxide (5 ml) and Copper (II) sulfate (2.75 mg in 50 μL water) or Iron (II) sulfate (2.75 mg in 50 μL water) were added to the reaction mixture and mixed thoroughly. The reaction in the capped reaction vessel proceeded in the shaker-incubator at 55° C. and 65 RPM for two hours. The capped reaction cooled to 12° C. in a −20° C. freezer. Ammonium hydroxide (1 ml of 28% v/v to pH 10.2) was used to adjust pH to 8, 9, or 10 and the sample was reacted at 45° C. in a shaker-incubator for 45 min, 60 min, or 90 min at 20 RPM, the cap was left loose to allow oxygen, ammonia, and carbon dioxide gases to be released. The sample was then frozen and lyophilized.

The freeze-dried oligosaccharide mixture was rehydrated with the minimum amount of water required to allow for a free-flowing solution. This solution was then loaded onto a column containing 15 mL mixed bed ion exchange resin per gram (dry weight) of crude material, and the runoff was collected in a plastic freezer bag. Once the material was loaded onto the column, the column was then rinsed with 3 bed volumes of water. Finally, the runoff was sealed and frozen in the bag, then carefully shattered and subjected to lyophilization.

Carbohydrate analysis: Post-cleavage oligosaccharide samples were reconstituted in water and subjected to alditol reduction. Samples were reduced for 1 hour with 2M sodium borohydride at 65° C., then immediately underwent C18 solid phase extraction. Solid phase cartridges were washed with three volumes acetonitrile and two volumes water before samples were loaded and collected as the immediate flow-through. The C18 cartridge-extracted samples were then subjected to non-porous graphitized carbon (NPGC) solid phase extraction. NPGC cartridges were sequentially pre-washed with two volumes water, two volumes of 80% acetonitrile with 0.01% (v/v) TFA in water, and two more volumes of water. The C18 cartridge-extracted samples were then loaded and washed with five volumes of water before being eluted with 40% acetonitrile with 0.05% (v/v) TFA. Finally, the post-NPGC samples were completely dried by evaporative centrifugation and stored at −20° C. until analysis.

(Amicucci, Galermo et al. 2019). Oligosaccharide analysis was carried out on an Agilent 1290 Infinity II HPLC coupled to an Agilent 6530 Accurate-Mass Q-TOF MS. Chromatographic separation was performed on a Thermo Scientific Hypercarb PGC column with a binary gradient which consisted of solvent A: 3% acetonitrile/water+0.1% formic acid and solvent B: 10% water/acetonitrile+0.1% formic acid. With a flow rate of 0.15 mL/min, the gradient was run for 60 min: 2-15% B, 0-20 min; 15-60% B, 20-45 min; 60-99% B, 45-45.10 min; 99-99% B, 45.10-51 min; 99-2% B, 51-51.10 min; 2-2% B, 51.10-60 min. The mass spectrometer was run in positive mode, with a reference mass of 922.0098 m/z. The gas temperature and flow rate were set to 150° C. and 11 1/min, respectively. The nozzle, fragmentor, skimmer voltages were set to be 1500, 75 and 60 volts, respectively. Using tandem mass spectrometry, fragmentation was performed with collision energy of 1.45×(m/z)−3.5. Data was processed using Agilent MassHunter Workstation Quantitative Analysis 10.1 Software. Major peaks in the chromatograms that corresponded to oligosaccharide masses were integrated.

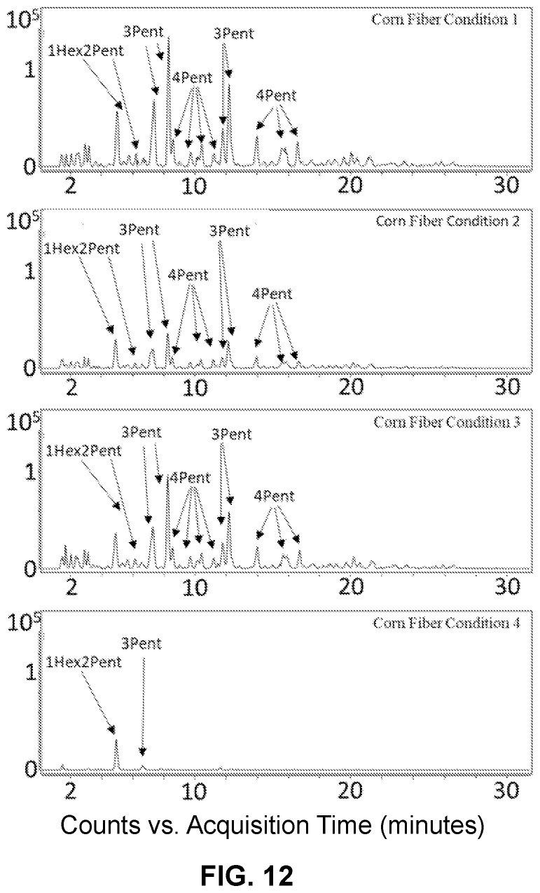

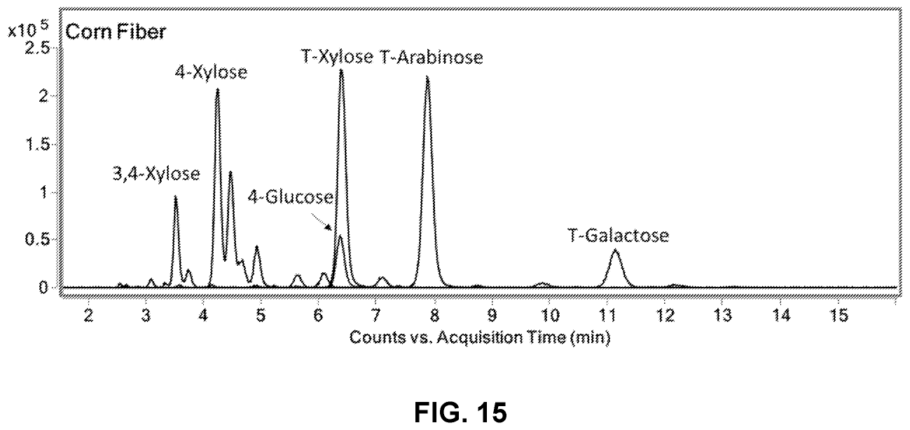

Results: The oligosaccharides produced from the Cu(II) oxidation and cleavage of corn fiber at pH 10 for 60 min proved to be the most successful at producing oligosaccharides (FIG. 12). The oligosaccharides ranged from DP 3-4 and had monosaccharide and linkage profiles that represented arabinoxylan. The oligosaccharides had a ratio of 1.36:1 xylose:arabinose and had linkages that include terminal xylose, terminal arabinose, terminal galactose, 4-xylose, 3,4-xylose and 4-glucose. For reference, an annotated linkage analysis chromatogram of corn fiber is provided in FIG. 15. FIG. 12 shows four unique corn fiber oligosaccharide profiles. Condition 1 produced the most oligosaccharides and resulted from the addition of NH₄OH to reach pH 10, where the solution was heated for 1 hour at 45° C. post Fenton oxidation with Cu (II). Condition 2 produced roughly 5-fold less oligosaccharides than Condition 1, which resulted from the addition of NH₄OH to reach pH 9, where the solution was heated for 1.5 hour at 45° C. post Fenton oxidation with Cu (II). Condition 3 produced slightly less oligosaccharides than Condition 1 and resulted from the addition of NH₄OH to reach pH 8, where the solution was heated for 0.75 hour at 45° C. post Fenton oxidation with Cu (II). These results indicate the pH, time, and temperature are important factors for optimizing the oligosaccharide yield. Furthermore, Condition 4 produced only few oligosaccharides and resulted from the addition of NH₄OH to reach pH 10, where the solution was heated for 1 hour at 45° C. post Fenton oxidation with Fe (II). This result indicates that some polysaccharide sources are more amenable to depolymerization when copper is used in the oxidation step, rather than iron.

### Example 10A Pipeline 1

### (Composition of Matter)

A number of polysaccharide rich materials were assessed for their ability to be dissociated by COG. Each material produced a number of unexpected oligosaccharide products, due to the prior lack of mechanism, and were characterized at both the pool level (multiple oligosaccharides) and the individual oligosaccharide level. When possible, the pools are described by their monosaccharide and glycosidic linkage profiles, 2D-NMR (Table 5A), and liquid chromatography/quadrapole-time-of-flight mass spectrometry (LC/Q-TOF MS). Furthermore, individual oligosaccharides were identified and characterized by their mass, retention time, and fragmentation patterns.

Oligosaccharide production: Arabinogalactan II, Lichenan, 1,4 B-Mannan, Xylan, Amylopectin, Arabinoxylan, Beta-Glucan, Galactan, Galactomannan Glucomannan, Xyloglucan, and Locust Bean Gum (550 mg) were dissolved in 20 ml of HPLC grade water in a capped reaction vessel and placed in a shaker-incubator for 20 min at 55° C. and 85 RPM. The pH of the solution was adjusted to 5.2. Hydrogen peroxide (5 ml) and iron (II) sulfate (2.75 mg in 50 μL water) were added to the reaction mixture and mixed thoroughly. The reaction in the capped reaction vessel proceeded in the shaker-incubator at 55° C. and 65 RPM for two hours. The capped reaction cooled to 12° C. in a −20° C. freezer. Ammonium hydroxide (1 ml of 28% v/v to pH 10.2) was used to adjust pH and sample was reacted at 45° C. in a shaker-incubator for 1 hour at 20 RPM, the cap was left loose to allow oxygen, ammonia, and carbon dioxide gases to be released. The sample is then frozen and lyophilized, then stored at −80° C. The freeze-dried oligosaccharide mixture was rehydrated with the minimum amount of water required to allow for a free-flowing solution. This solution was then loaded onto a column containing 15 mL mixed bed ion exchange resin per gram (dry weight) of crude material, and the runoff was collected in a plastic freezer bag. Once the material was loaded onto the column, the column was then rinsed with 3 bed volumes of water. Finally, the runoff was sealed and frozen in the bag, then carefully shattered and subjected to lyophilization.

Curdlan and Corn Fiber (550 mg) were dissolved in 20 ml of HPLC grade water in a capped reaction vessel and placed in a shaker-incubator for 20 min at 55° C. and 85 RPM. The pH of the solution was adjusted to 5.2. Hydrogen peroxide (5 ml) and Cu (II) sulfate (2.75 mg in 50 μL water) were added to the reaction mixture and mixed thoroughly. The reaction in the capped reaction vessel proceeded in the shaker-incubator at 55° C. and 65 RPM for two hours. The capped reaction cooled to 12° C. in a −20° C. freezer. Ammonium hydroxide (1 ml of 28% v/v to pH 10.2) was used to adjust pH and sample was reacted at 45° C. in a shaker-incubator for 1 hour at 20 RPM, the cap was left loose to allow oxygen, ammonia, and carbon dioxide gases to be released. The sample is then frozen and lyophilized, then stored at −80° C. The freeze-dried oligosaccharide mixture was rehydrated with the minimum amount of water required to allow for a free-flowing solution. This solution was then loaded onto a column containing 15 mL mixed bed ion exchange resin per gram (dry weight) of crude material, and the runoff was collected in a plastic freezer bag. Once the material was loaded onto the column, the column was then rinsed with 3 bed volumes of water. Finally, the runoff was sealed and frozen in the bag, then carefully shattered and subjected to lyophilization.

Monosaccharide analysis was performed in the manner of Amicucci et al. (Amicucci, M. J., Galermo, A. G., et al. (2019). International Journal of Mass Spectrometry 438: 22-28.) but was adapted to be run on an Agilent 6530 Q-TOF mass spectrometer. Glycosidic linkage analysis was performed in the manner of Galermo, A. G. Nandita, E. et al (2018 Analytical Chemistry 90(21): 13073-13080 with the expanded retention time library presented in Galermo, A. G., Nandita, E., et al. (2019). Analytical Chemistry 91(20): 13022-13031 and was adapted to be run on an Agilent 6530 Q-TOF mass spectrometer. Oligosaccharide analysis was performed in the manner of Amicucci, M. J., Nandita, E., et al. (2020). Nature Communications 11(1): 1-12. Oligosaccharide peak volumes were generated from Agilent Mass Hunter Qualitative Analysis B.10 by using their “find by molecular feature” function. For NMR analysis, oligosaccharides were dissolved in D₂O at a concentration of 50 mg/ml and were analyzed on a 600 MHz Bruker NMR spectrometer for their HSQC spectra.

Monosaccharide Composition: The oligosaccharide pools generated from the COG reaction were analyzed for their monosaccharide compositions, which are shown in Table 3A. Seven monosaccharides were measured in the 15 samples that underwent the COG reactions.

TABLE 3A
Monosaccharide composition of claimed composition of matter pools. Units represent
relative abundance by mass. The notation “—” represents a monosaccharide
that exists in an amount less than 2% (which can be 0%) of the total polysaccharide weight.

| | Glucose | Galactose | Mannose | Arabinose | Xylose | Rhamnose | GalA | Others |
|---|---|---|---|---|---|---|---|---|
| Arabinogalactan II | — | 87.28 | — | 7.23 | — | — | — | 1.27 |
| Lichenan | 80.21 | 8.64 | 8.64 | — | — | — | — | 1.11 |
| 1,4 B-Mannan | 4.48 | 7.61 | 83.8 | 2.99 | — | — | — | 1.11 |
| Xylan | 5.36 | 2.04 | 4.9 | — | 85.48 | — | — | 0.78 |
| Amylopectin | 98.19 | — | — | — | — | — | — | 0.54 |
| Arabinoxylan | — | 2.08 | — | 36.99 | 60.28 | — | — | 0.09 |
| Beta-Glucan | 97.04 | — | — | — | — | — | — | 1.61 |
| (Barley) | | | | | | | | |
| Galactan | — | 80.06 | — | 9.28 | — | 4.59 | 3.04 | 2.44 |
| Galactomannan | — | 18.91 | 78.14 | — | — | — | — | 0.3 |
| Glucomannan | 36.73 | — | 60.45 | — | — | — | — | 0.19 |
| Xyloglucan | 48.75 | 14.14 | — | — | 36.38 | — | — | 0.48 |
| Locust Bean Gum | — | 22.98 | 72.91 | — | — | — | — | 0.33 |
| Curdlan | 99.04 | — | — | — | — | — | — | 0.28 |
| Corn Fiber | 3.07 | 6.78 | — | 35.76 | 48.68 | — | — | 4.65 |
| Beta-Glucan (Oat) | 95.01 | — | — | 2.38 | — | — | — | 0.74 |

Glycosidic Linkage Analysis: The oligosaccharide pools generated from the COG reaction were analyzed for their monosaccharide compositions, which are shown in Table 4A. Sixteen glycosidic linkage positions were identified in the 15 samples that underwent the COG reactions.

TABLE 4A
Glycosidic linkage compositions of claimed composition of matter pools. Units represent
relative abundance by peak area. The notation “—” represents a glycosidic linkage
that exists in an amount less than 2% (which can be 0%) of the total polysaccharide weight.

| | | | | 4,6- | | | | 3,6- | | |
|---|---|---|---|---|---|---|---|---|---|---|
| | 3-Glc | 4-Glc | 6-Glc | Glc | T-Glc | 3-Gal | 4-Gal | Gal | 6-Gal | T-Gal |
| Arabinoxylan | — | — | — | — | — | — | — | — | — | — |
| Xyloglucan | — | 28.23 | 5.63 | 20.49 | 4.23 | — | — | — | — | 20.62 |
| Beta-glucan | 17.06 | 48.91 | — | — | 30.95 | — | — | — | — | — |
| (Barley) | | | | | | | | | | |
| Amylopectin | — | 70.23 | — | 3.83 | 17.60 | — | — | — | — | — |
| Galactomannan | — | 2.34 | — | — | — | — | — | — | — | 17.85 |
| Arabinogalactan II | — | — | — | — | — | 17.33 | — | 14.18 | 11.83 | 50.75 |
| Lichenan | 25.57 | 42.76 | — | — | 22.71 | — | — | — | — | 7.45 |
| Mannan | — | — | — | — | — | — | — | — | — | 3.63 |
| Xylan | — | 13.61 | — | — | 5.19 | — | — | — | — | — |
| Galactan | — | — | — | — | — | — | 61.68 | — | — | 33.73 |
| Glucomannan | — | 31.52 | — | — | 7.65 | — | — | — | — | — |
| Locust Bean Gum | — | — | — | — | — | — | — | — | — | 19.58 |
| Curdlan | 74.84 | — | — | — | 8.82 | — | — | — | — | — |
| Corn Fiber | — | 5.83 | — | — | — | — | — | — | — | 6.49 |
| Beta-glucan (Oat) | 23.04 | 64.07 | 0.25 | — | 12.64 | — | — | — | — | — |

| | | | 3,4- | | | | 4,6- | | |
|---|---|---|---|---|---|---|---|---|---|
| | 2-Xyl | 4-Xyl | Xyl | T-Xyl | T-Arab | 4-Man | Man | T-Man | Other |
| Arabinoxylan | — | 31.20 | 22.22 | 2.65 | 30.55 | — | — | — | 11.14 |
| Xyloglucan | 5.81 | — | — | 10.78 | — | — | — | — | 0.97 |
| Beta-glucan | — | — | — | — | — | — | — | — | 1.36 |
| Amylopectin | — | — | — | — | — | — | — | — | 4.82 |
| Galactomannan | — | — | — | — | — | 47.34 | 6.52 | 20.79 | 4.39 |
| Arabinogalactan II | — | — | — | — | 3.28 | — | — | — | 1.09 |
| Lichenan | — | — | — | — | — | — | — | — | 0.0 |
| Mannan | — | — | — | — | — | 58.31 | — | 34.60 | 0.0 |
| Xylan | — | 54.71 | — | 7.18 | — | 15.28 | — | — | 1.93 |
| Galactan | — | — | — | — | 2.02 | — | — | — | 1.62 |
| Glucomannan | — | — | — | — | — | 47.58 | | 12.58 | 0.0 |
| Locust Bean Gum | — | — | — | — | — | 62.02 | 8.95 | 6.82 | 0.0 |
| Curdlan | — | — | — | — | — | — | — | — | 15.13 |
| Corn Fiber | — | 16.33 | 6.21 | 25.05 | 27.79 | — | — | — | 12.29 |
| Beta-glucan (Oat) | — | — | — | — | — | — | — | — | 0.0 |

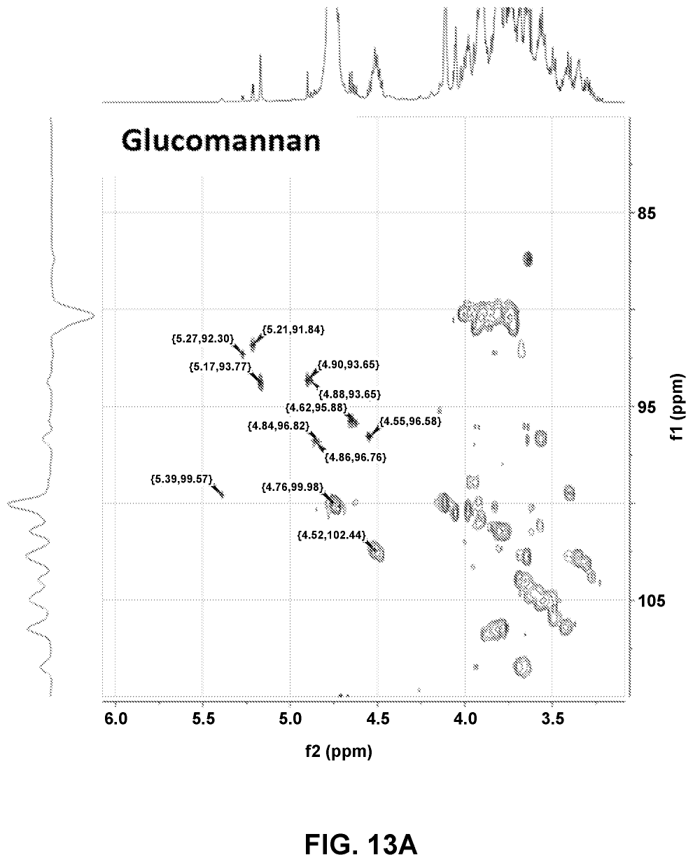

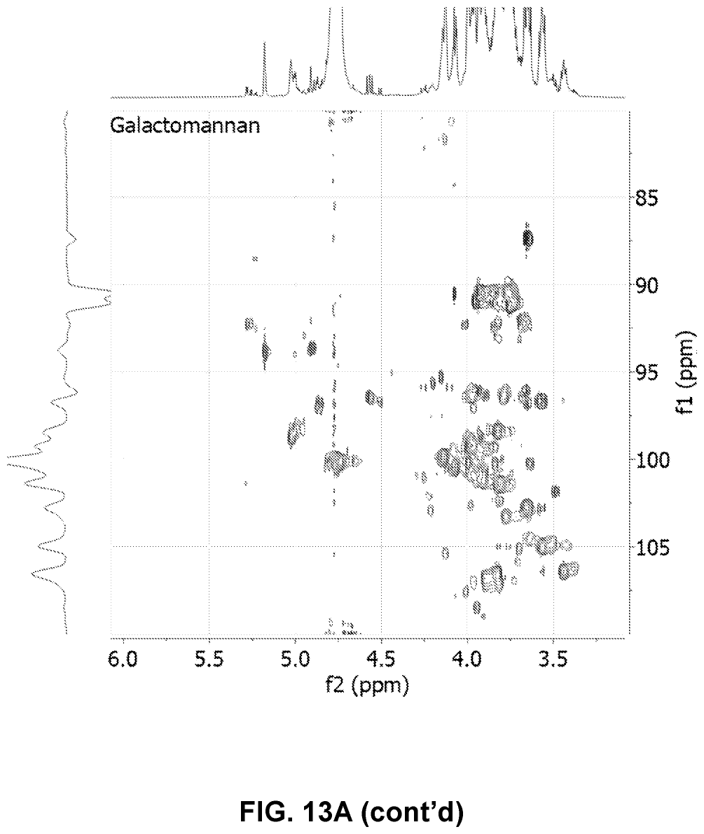

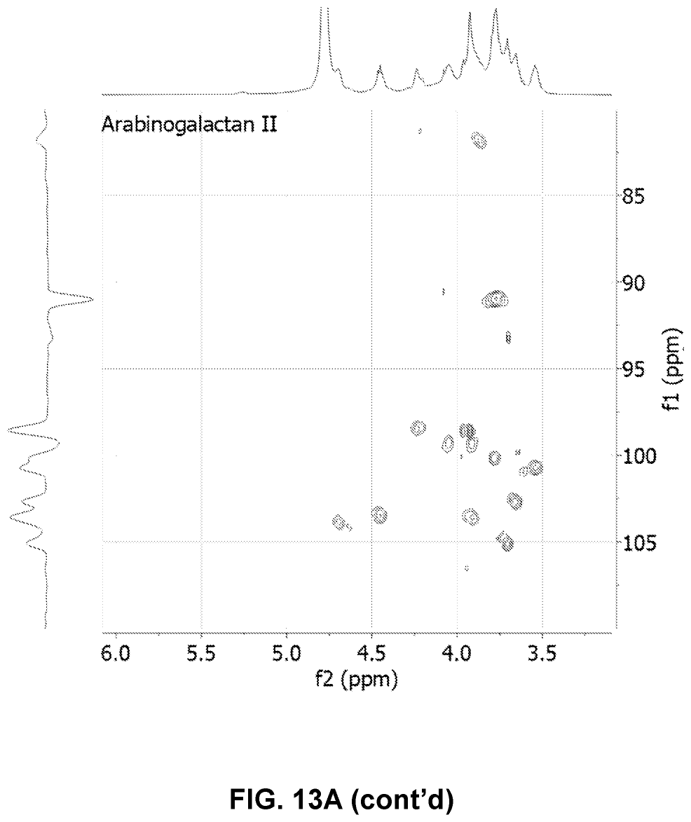

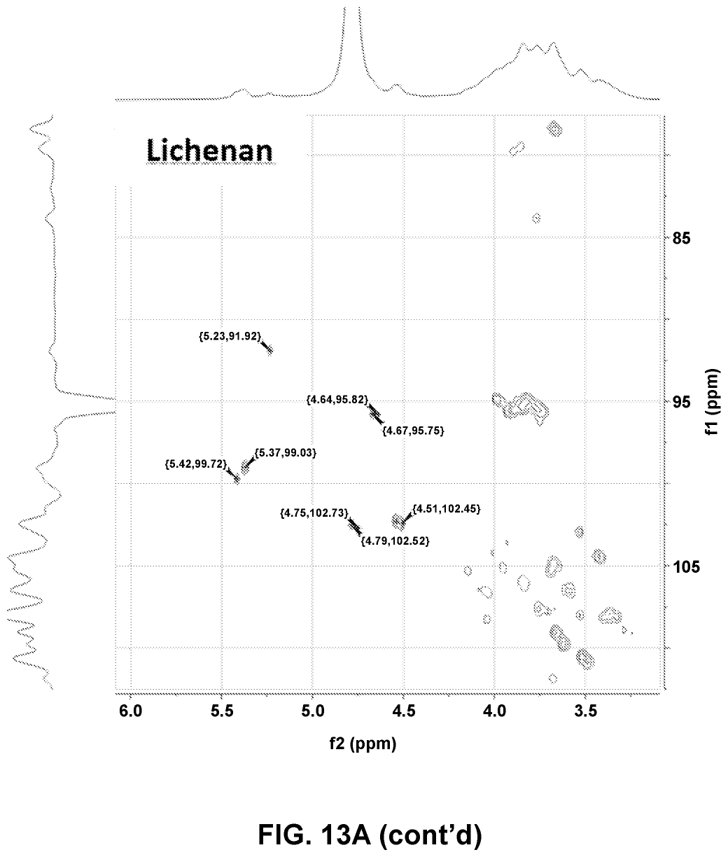

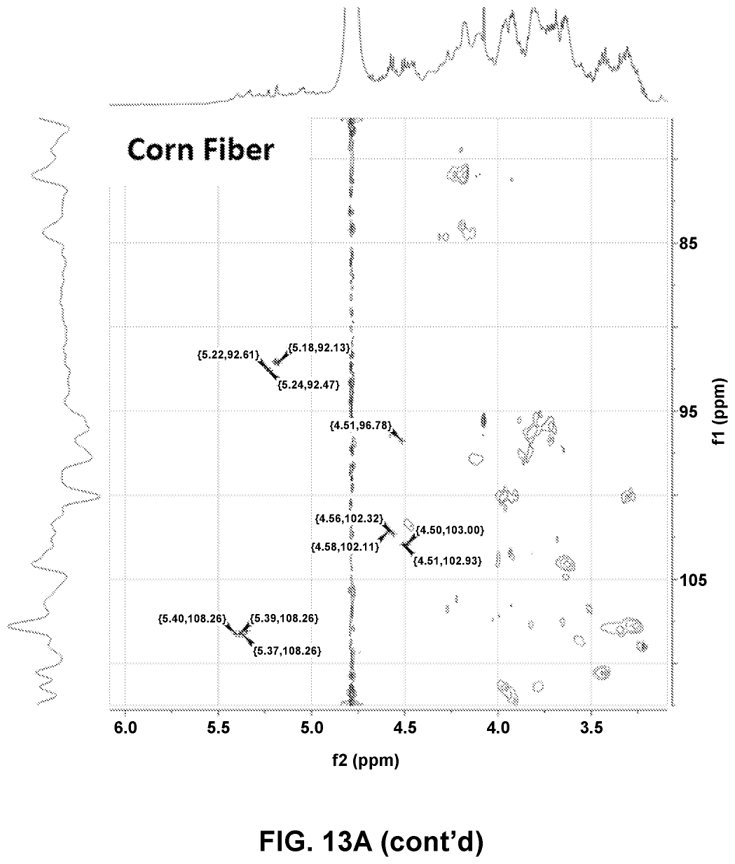

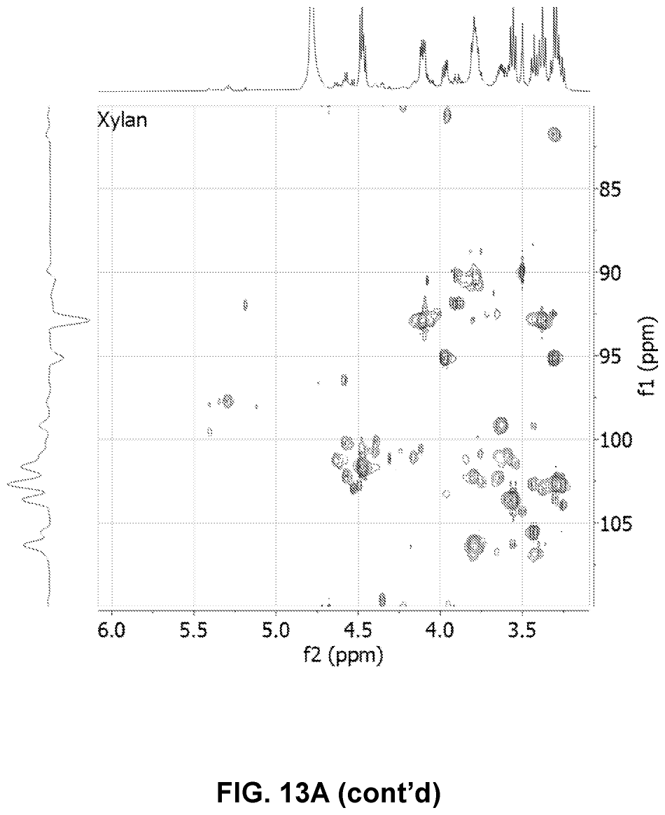

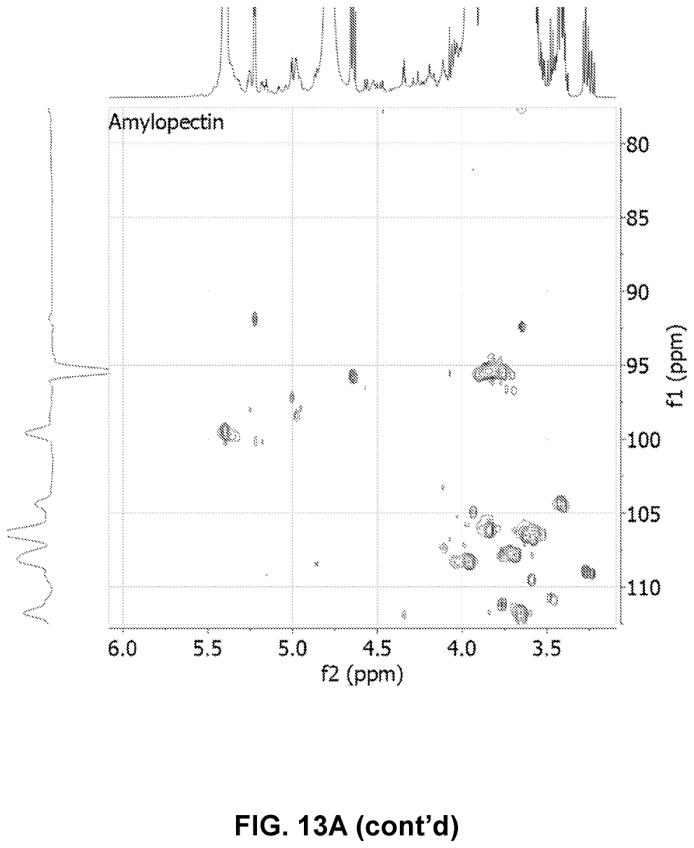

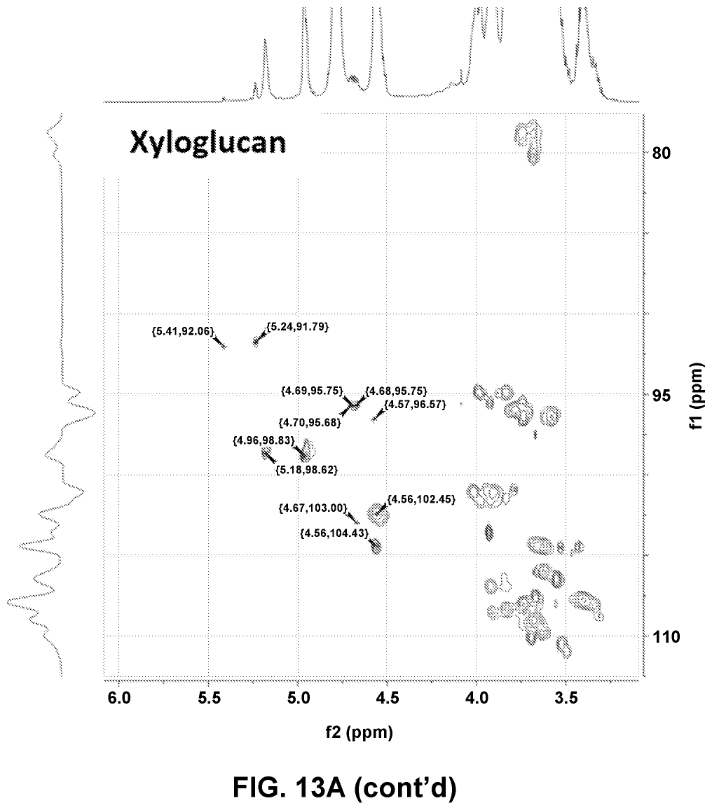

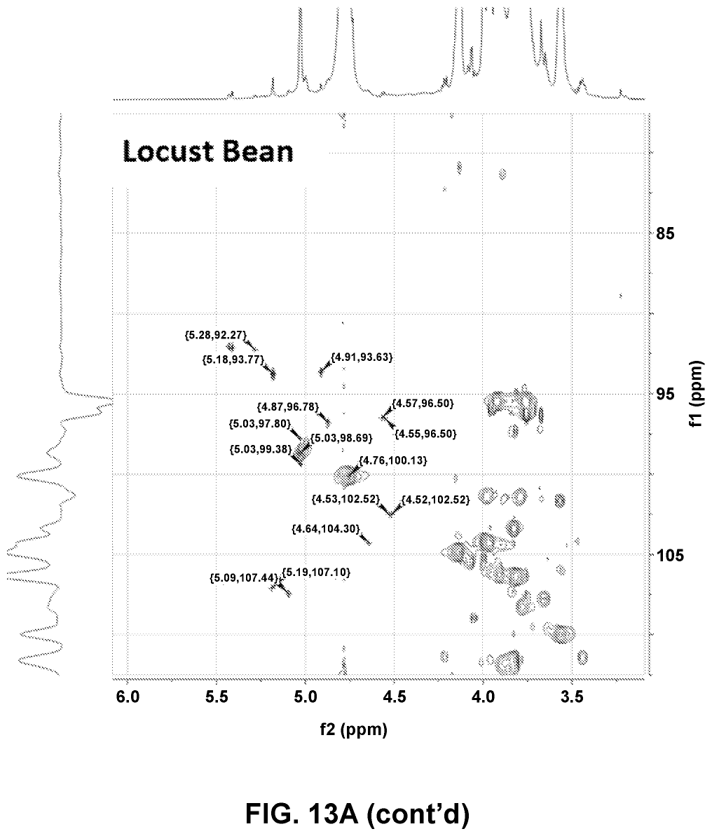

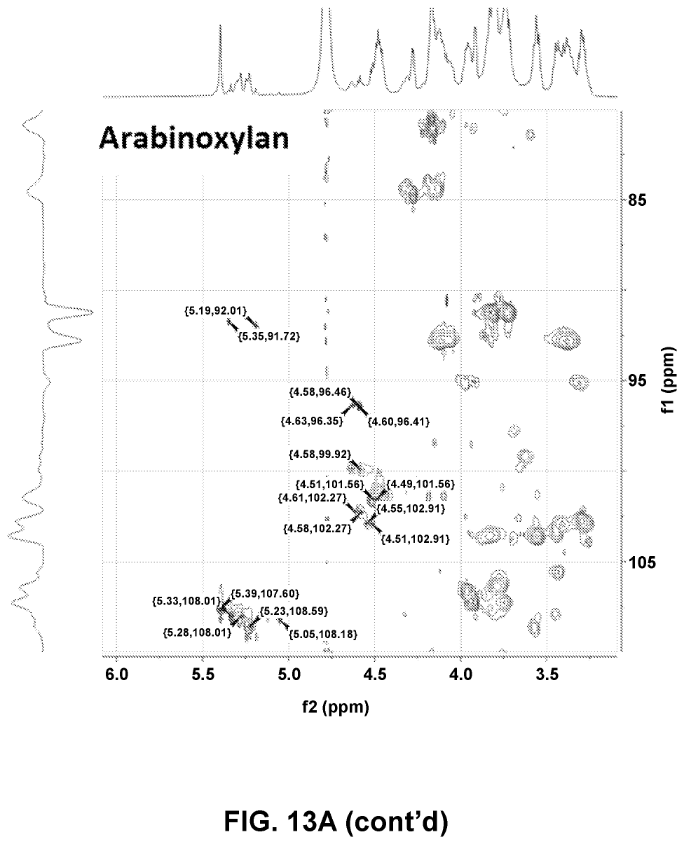

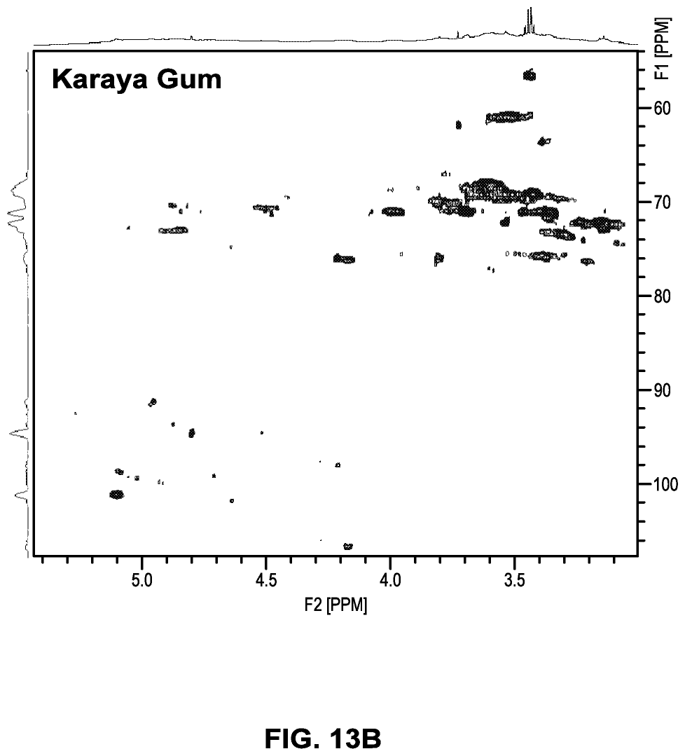

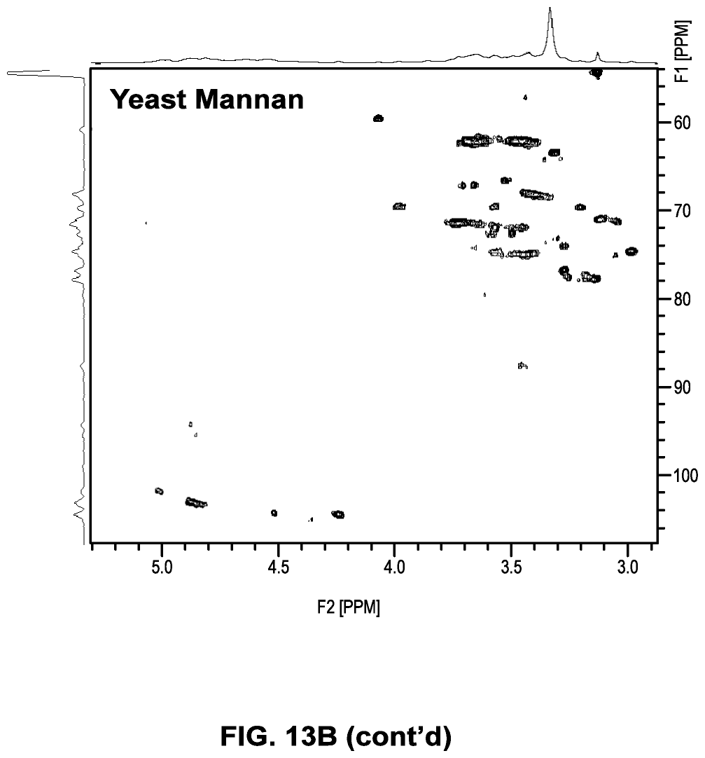

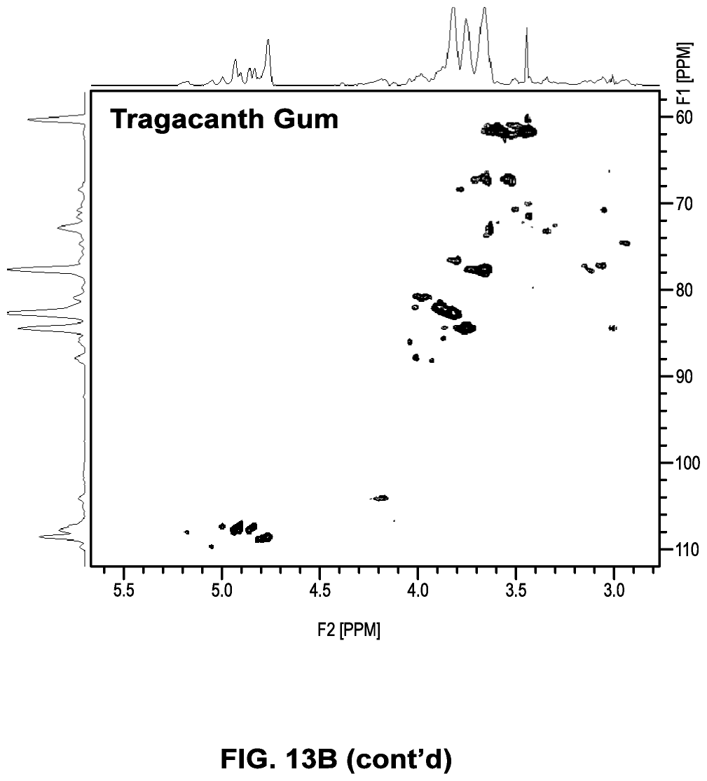

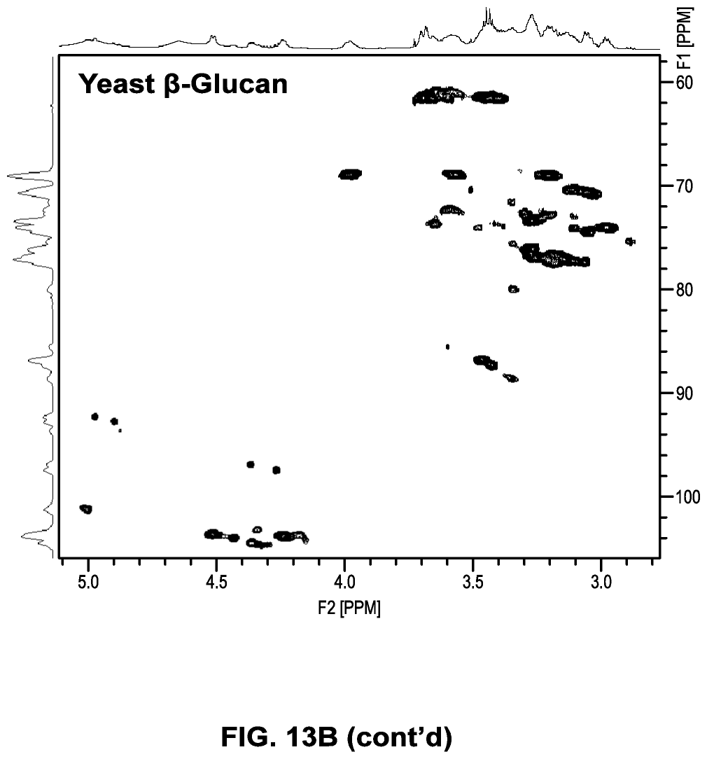

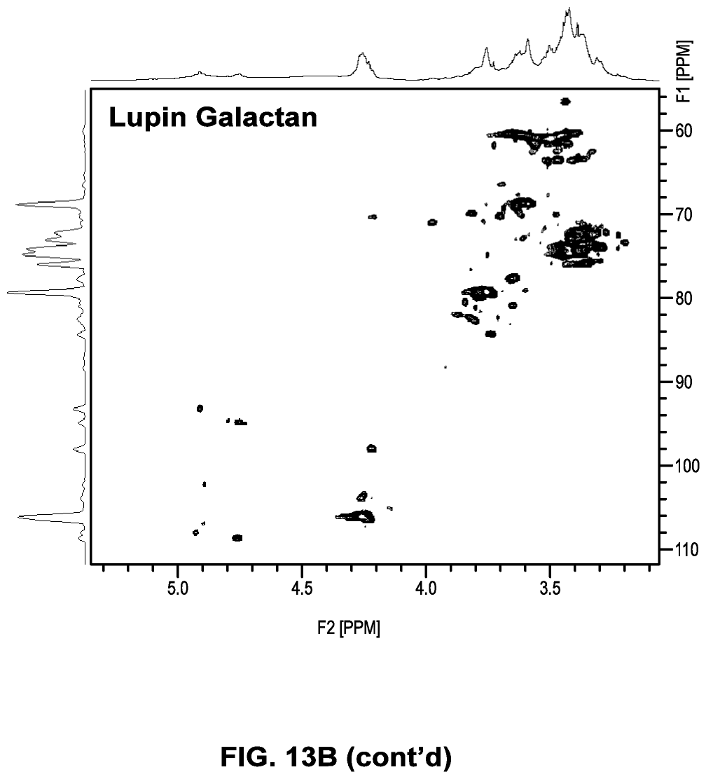

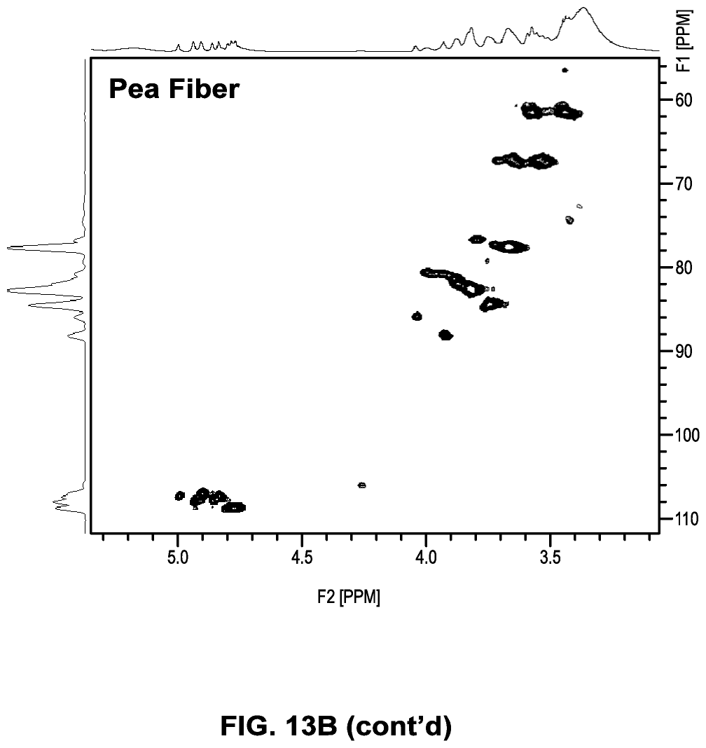

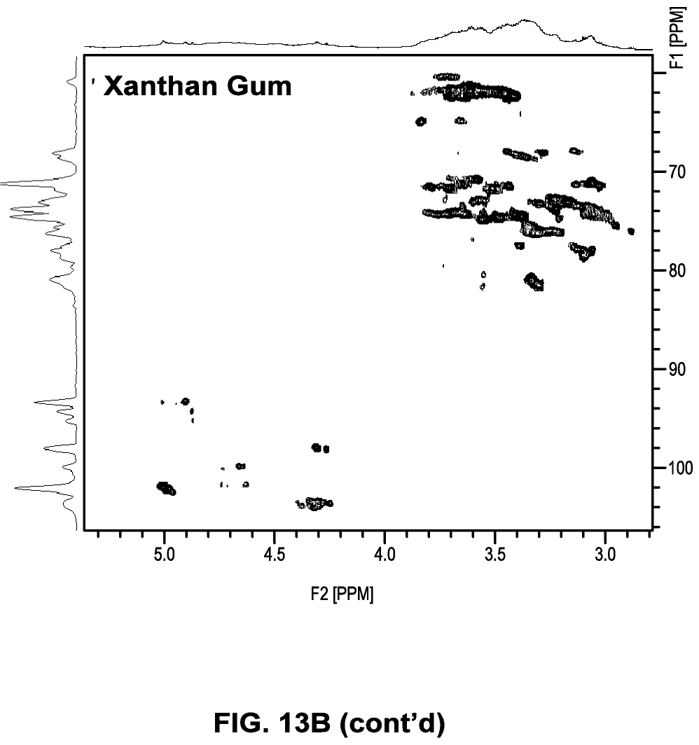

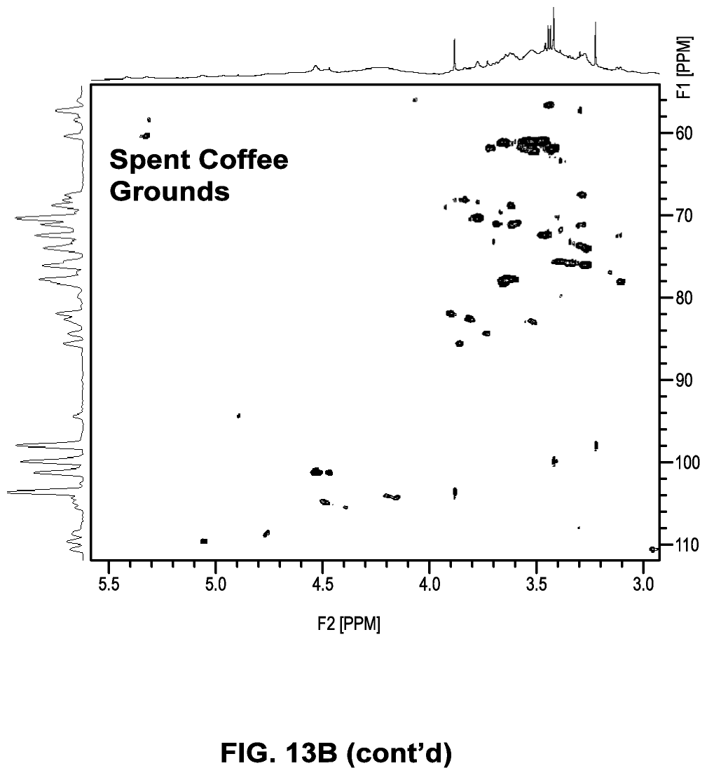

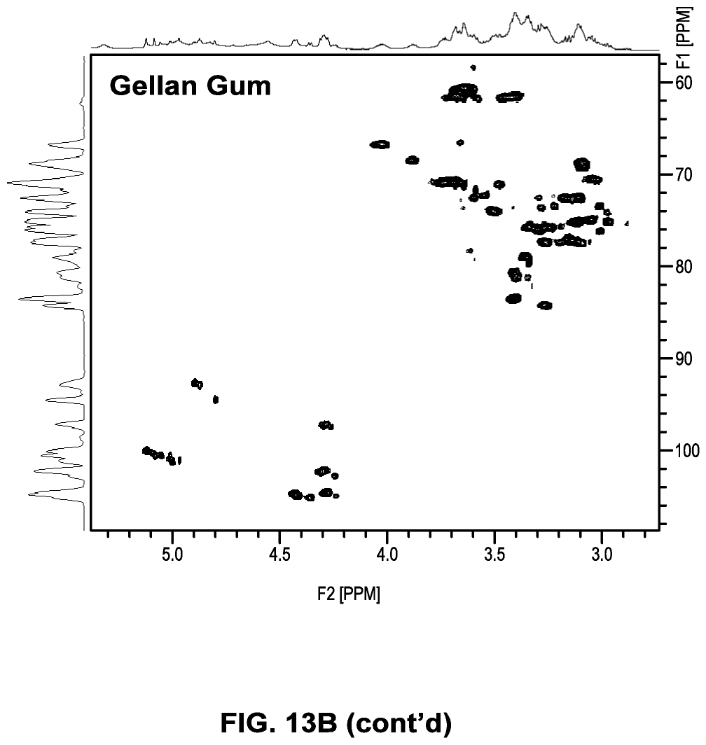

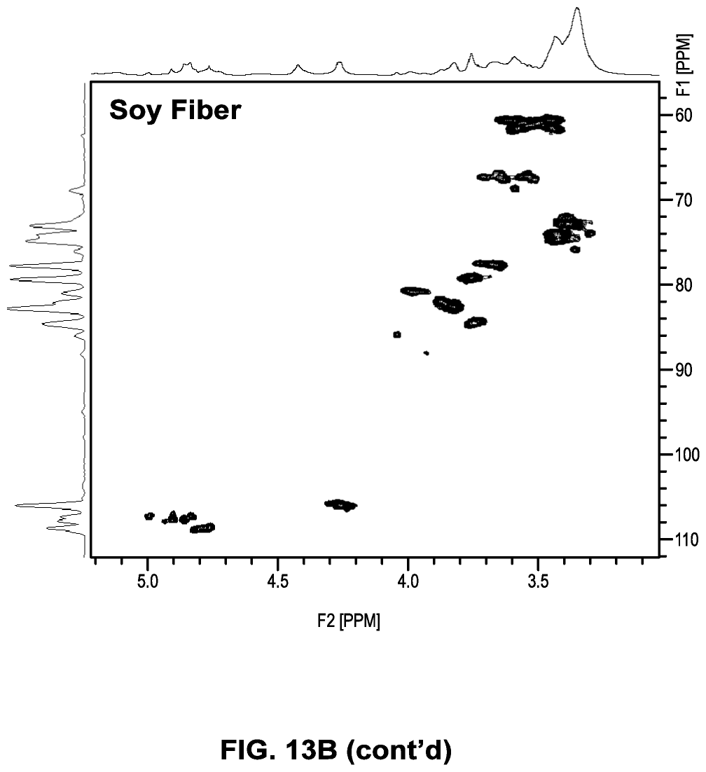

1H-13C HSQC NMR: Was performed on all of the samples. The analysis provided a fingerprint of each sample in order to compare the similarities between these and future oligosaccharide pools. The cross peak coordinates found in the anomeric region of the spectra are listed in Table 5A and some of the spectra are provided in FIG. 13A.

TABLE 5A
1H-13C HSQC NMR correlations from oligosaccharides created from the COG process.
The listed pairs correspond to those major peaks in the anomeric region.

| Microbial | | Konjac | | Carob | | Barley β- | | Arabinogalactan | | | |
|---|---|---|---|---|---|---|---|---|---|---|---|
| Curdlan | | Glucomannan | | Galactomannan | | Glucan | | II | | Lichenan | |
| 1H δ | 13C δ | 1H δ | 13C δ | 1H δ | 13C δ | 1H δ | 13C δ | 1H δ | 13C δ | 1H δ | 13C δ |
| [ppm] | [ppm] | [ppm] | [ppm] | [ppm] | [ppm] | [ppm] | [ppm] | [ppm] | [ppm] | [ppm] | [ppm] |
| 3.37 | 103.44 | 4.52 | 102.44 | 4.50 | 96.76 | 4.64 | 95.88 | 3.54 | 100.74 | 4.51 | 102.45 |
| 3.42 | 99.57 | 4.55 | 96.58 | 4.51 | 96.70 | 4.75 | 102.73 | 3.64 | 99.80 | 4.54 | 102.32 |
| 3.52 | 105.61 | 4.56 | 96.52 | 4.55 | 96.52 | 4.67 | 95.70 | 3.65 | 99.80 | 4.64 | 95.82 |
| 3.52 | 98.05 | 4.62 | 95.88 | 4.57 | 96.46 | 4.76 | 102.68 | 3.66 | 102.68 | 4.66 | 95.75 |
| 3.57 | 103.32 | 4.65 | 95.76 | 4.58 | 96.41 | 4.78 | 102.50 | 3.70 | 93.24 | 4.67 | 95.75 |
| 3.74 | 90.66 | 4.66 | 95.76 | 4.85 | 96.82 | 4.79 | 102.50 | 3.71 | 105.14 | 4.75 | 102.73 |
| 3.76 | 90.61 | 4.76 | 99.98 | 4.87 | 96.76 | 5.23 | 91.89 | 3.71 | 105.08 | 4.79 | 102.52 |
| 3.80 | 84.04 | 4.84 | 96.82 | 4.89 | 93.65 | 4.65 | 95.88 | 3.77 | 90.96 | 5.23 | 91.92 |
| 3.92 | 90.66 | 4.86 | 96.76 | 4.91 | 93.65 | 4.52 | 102.50 | 3.78 | 100.16 | 5.37 | 99.03 |
| 3.95 | 90.66 | 4.88 | 93.65 | 5.00 | 98.34 | 4.51 | 102.50 | 3.86 | 81.93 | 5.42 | 99.72 |
| 4.64 | 95.88 | 4.90 | 93.65 | 5.00 | 94.00 | 4.54 | 102.32 | 3.91 | 103.61 | | |
| 4.65 | 95.88 | 5.17 | 93.77 | 5.03 | 98.69 | 4.66 | 95.70 | 3.91 | 99.22 | | |
| 4.67 | 95.64 | 5.21 | 91.84 | 5.18 | 93.83 | | | 3.93 | 98.63 | | |
| 4.69 | 95.64 | 5.27 | 92.30 | 5.23 | 92.54 | | | 3.96 | 98.57 | | |
| 4.76 | 102.73 | 5.39 | 99.57 | 5.23 | 88.55 | | | 4.05 | 99.22 | | |
| 4.77 | 102.68 | | | 5.24 | 88.55 | | | 4.20 | 98.40 | | |
| 4.80 | 102.50 | | | 5.26 | 92.25 | | | 4.24 | 98.46 | | |
| 4.81 | 102.50 | | | 5.28 | 92.30 | | | 4.45 | 103.44 | | |
| 5.24 | 92.01 | | | 5.29 | 101.39 | | | 4.69 | 103.85 | | |
| Corn Fiber | | Beechwood | | | | Tamarind | | Locust Bean Gum | | Rye | |
| Arabinoxylan | | Xylan | | Amylopectin | | Xyloglucan | | Galactomannan | | Arabinoxylan | |
| 1H δ | 13C δ | 1H δ | 13C δ | 1H δ | 13C δ | 1H δ | 13C δ | 1H δ | 13C δ | 1H δ | 13C δ |
| [ppm] | [ppm] | [ppm] | [ppm] | [ppm] | [ppm] | [ppm] | [ppm] | [ppm] | [ppm] | [ppm] | [ppm] |
| 4.49 | 102.93 | 4.16 | 101.09 | 4.26 | 100.40 | 4.56 | 104.43 | 4.52 | 102.52 | 4.46 | 101.27 |
| 4.50 | 103.00 | 4.31 | 101.15 | 4.29 | 100.40 | 4.56 | 102.45 | 4.53 | 102.52 | 4.48 | 101.56 |
| 4.51 | 102.93 | 4.35 | 109.59 | 4.56 | 96.57 | 4.57 | 96.57 | 4.55 | 96.50 | 4.49 | 101.56 |
| 4.51 | 96.78 | 4.39 | 100.10 | 4.57 | 96.50 | 4.67 | 103.00 | 4.57 | 96.50 | 4.51 | 102.91 |
| 4.56 | 102.32 | 4.40 | 100.74 | 4.64 | 95.75 | 4.67 | 95.68 | 4.64 | 104.30 | 4.51 | 101.56 |
| 4.58 | 102.11 | 4.46 | 101.80 | 4.65 | 95.75 | 4.68 | 95.75 | 4.76 | 100.13 | 4.55 | 102.91 |
| 5.18 | 92.13 | 4.48 | 101.62 | 4.85 | 108.47 | 4.69 | 95.75 | 4.87 | 96.78 | 4.58 | 102.27 |
| 5.19 | 92.06 | 4.48 | 100.86 | 4.87 | 108.40 | 4.70 | 95.68 | 4.91 | 93.63 | 4.58 | 99.92 |
| 5.22 | 92.61 | 4.49 | 102.79 | 4.95 | 97.94 | 4.96 | 98.83 | 5.03 | 99.38 | 4.58 | 96.46 |
| 5.24 | 92.47 | 4.49 | 101.62 | 4.98 | 98.42 | 5.18 | 98.62 | 5.03 | 98.69 | 4.60 | 96.41 |
| 5.35 | 108.06 | 4.49 | 100.86 | 5.00 | 97.12 | 5.24 | 91.79 | 5.03 | 97.80 | 4.61 | 102.27 |
| 5.37 | 108.26 | 4.50 | 102.79 | 5.04 | 98.90 | 5.41 | 92.06 | 5.09 | 107.44 | 4.62 | 96.35 |
| 5.39 | 108.26 | 4.53 | 102.91 | 5.08 | 100.81 | | | 5.18 | 93.77 | 4.63 | 99.86 |
| 5.40 | 108.26 | 4.54 | 102.91 | 5.15 | 109.15 | | | 5.19 | 107.10 | 4.63 | 96.35 |
| | | 4.55 | 100.21 | 5.18 | 100.13 | | | 5.28 | 92.27 | 5.05 | 108.18 |
| | | 4.57 | 102.21 | 5.22 | 100.13 | | | 5.41 | 92.13 | 5.19 | 92.01 |
| | | 4.57 | 100.21 | 5.22 | 91.86 | | | 5.43 | 92.06 | 5.23 | 108.59 |
| | | 4.58 | 102.21 | 5.25 | 100.74 | | | | | 5.28 | 108.01 |
| | | 4.58 | 96.46 | 5.25 | 97.94 | | | | | 5.33 | 108.01 |
| | | 4.59 | 100.27 | 5.26 | 92.61 | | | | | 5.35 | 91.72 |
| | | 4.59 | 96.46 | 5.40 | 99.58 | | | | | 5.39 | 107.60 |
| | | 4.63 | 101.27 | | | | | | | | |
| | | 4.64 | 101.27 | | | | | | | | |
| | | 4.71 | 101.68 | | | | | | | | |
| | | 4.72 | 101.74 | | | | | | | | |
| | | 4.73 | 96.64 | | | | | | | | |
| | | 4.74 | 96.64 | | | | | | | | |
| | | 5.06 | 101.39 | | | | | | | | |
| | | 5.06 | 98.46 | | | | | | | | |
| | | 5.09 | 96.93 | | | | | | | | |
| | | 5.12 | 98.05 | | | | | | | | |
| | | 5.19 | 91.95 | | | | | | | | |
| | | 5.29 | 97.75 | | | | | | | | |
| | | 5.30 | 97.75 | | | | | | | | |
| | | 5.40 | 99.57 | | | | | | | | |
| | | 5.41 | 97.93 | | | | | | | | |
| | | 5.41 | 92.13 | | | | | | | | |

| | |
|---|---|
| Oat | Pectic |
| β-Glucan | Galactan |

| 1H δ | 13C δ | 1H δ | 13C δ |
|---|---|---|---|
| [ppm] | [ppm] | [ppm] | [ppm] |
| 4.96 | 100.74 | 4.91 | 92.33 |
| 4.93 | 91.77 | 4.89 | 101.49 |
| 4.86 | 92.09 | 4.85 | 92.53 |
| 4.37 | 103.69 | 4.84 | 106.89 |
| 4.33 | 102.65 | 4.80 | 93.82 |
| 4.32 | 96.45 | 4.75 | 107.74 |
| 4.28 | 102.79 | 4.74 | 94.09 |
| 4.28 | 96.69 | 4.26 | 103.12 |
| 4.27 | 104.10 | 4.24 | 105.38 |
| 4.22 | 96.93 | 4.21 | 102.89 |
| 4.20 | 103.24 | 4.21 | 97.19 |
| | | 4.13 | 103.63 |

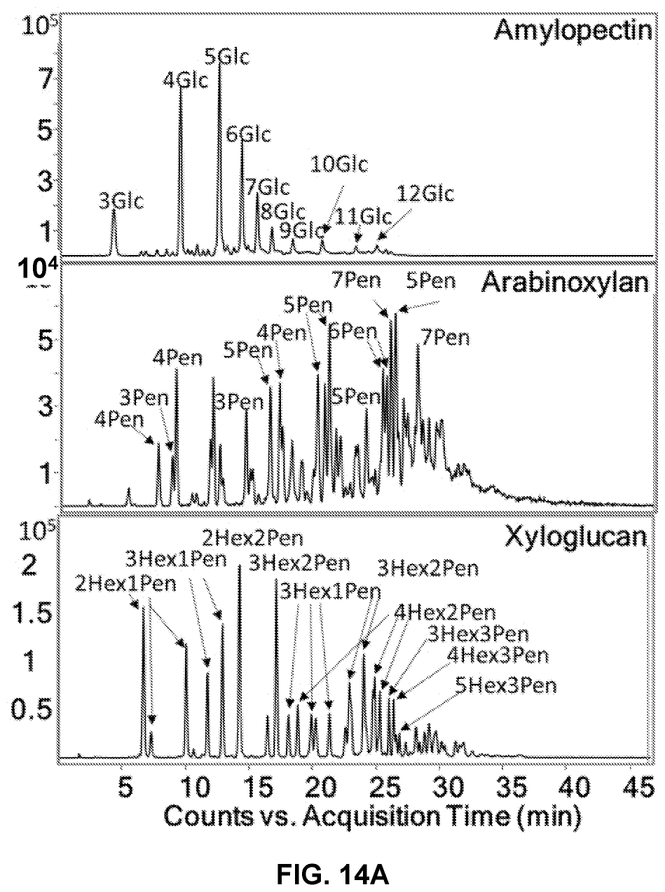

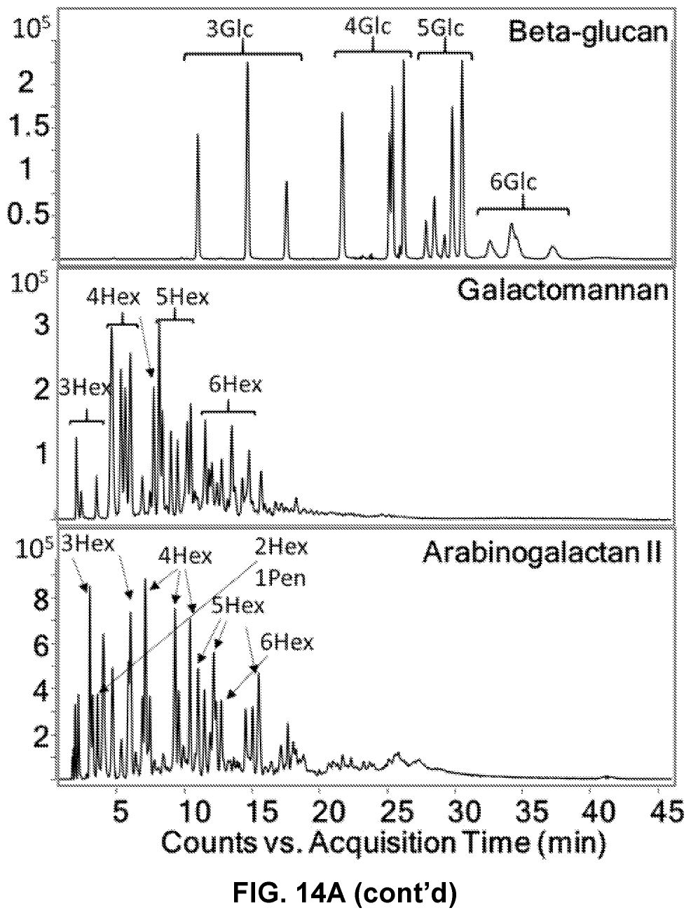

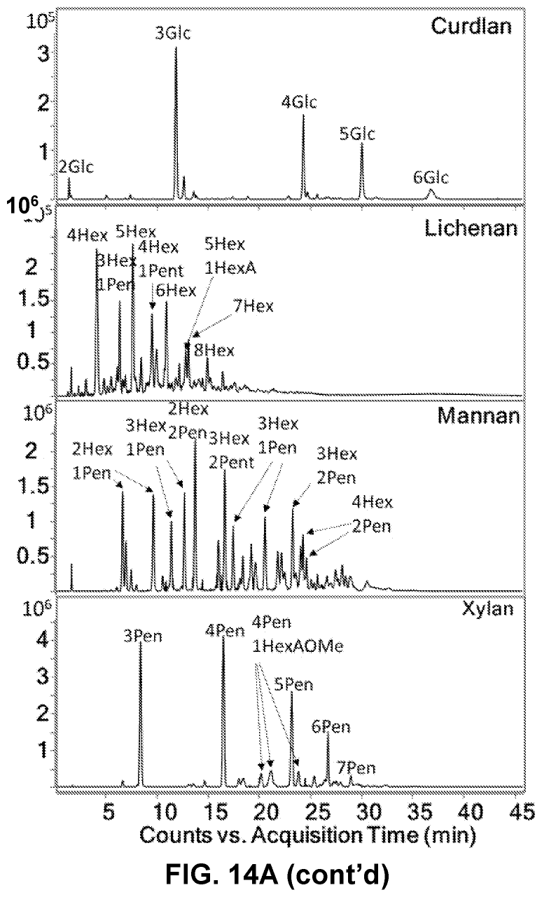

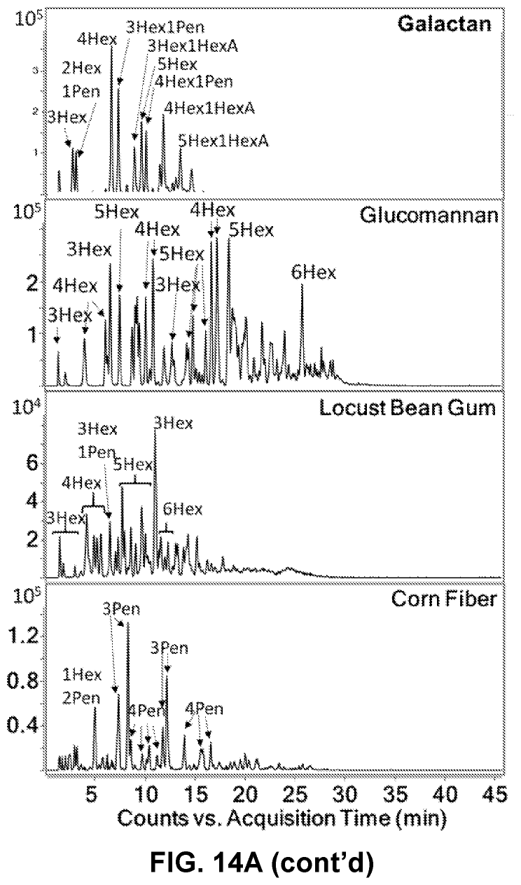

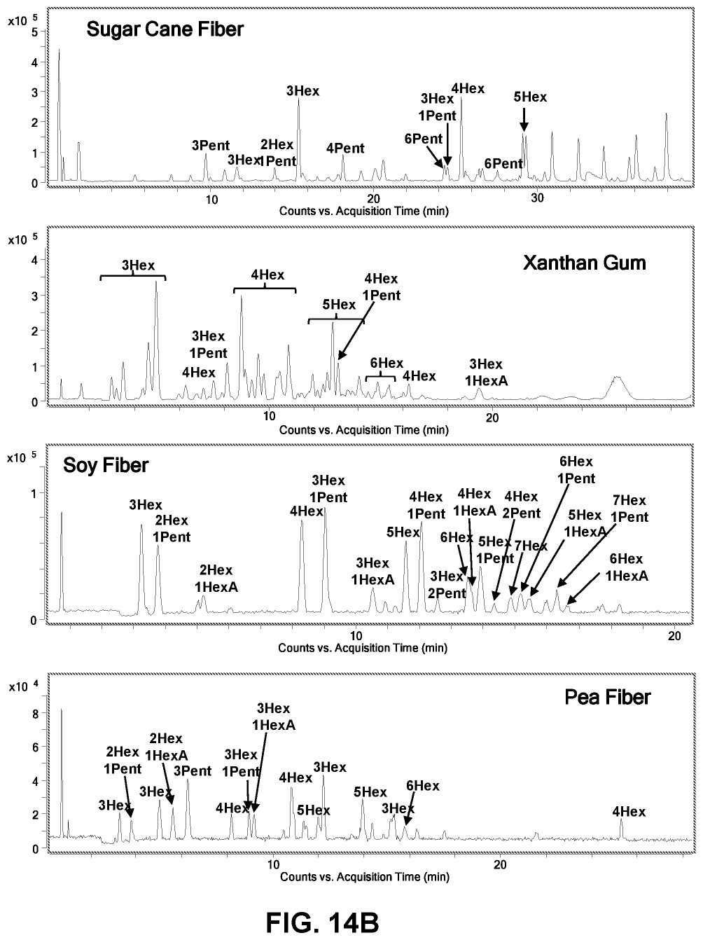

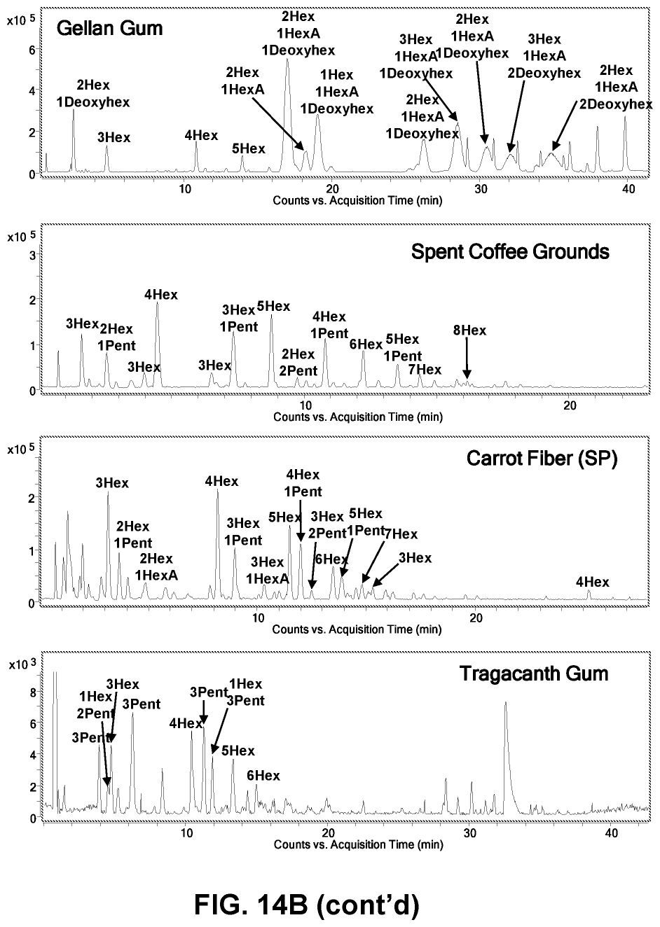

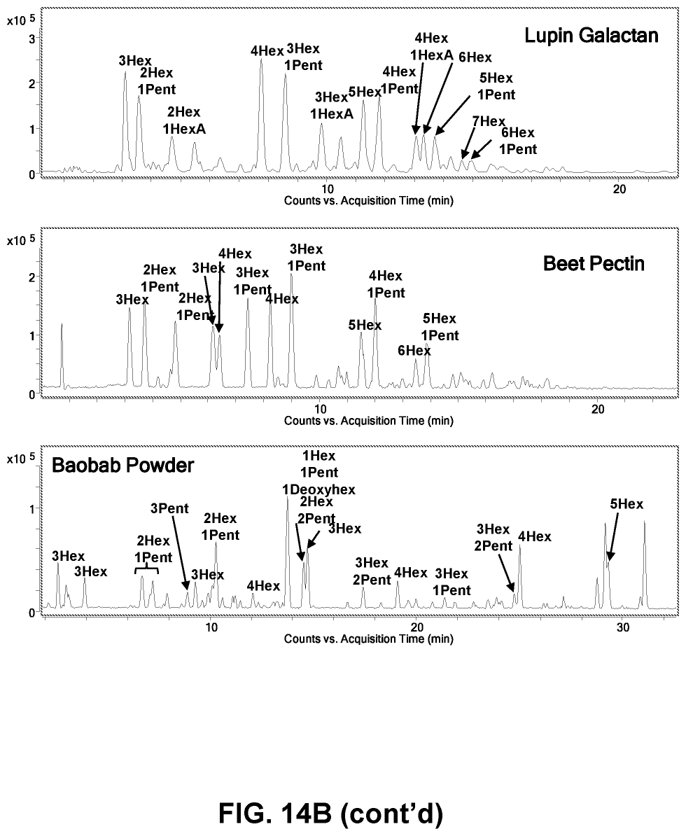

Oligosaccharide Analysis: Oligosaccharides are presented in two formats. Tables 6-20 show the “Find By Molecular Feature” data that shows the mass, retention time, composition, and oligosaccharide relative abundance. Additionally, we have provided annotated chromatograms of the oligosaccharides in FIG. 14A.

Amylopectin refers to a polysaccharide with an α-1,4 backbone with α-1,6 branches that extend in linear α-1,4 branches that may be similarly branched. The produced oligosaccharides possessed various structural features similar to those in the parent polysaccharide. The glucose composition was 98.19% (Table 3A) and a glycosidic linkage composition of 17.6% terminal-glucose, 70.23% 4-linked glucose, and 3.83% 4,6-linked glucose (Table 4A). 29 oligosaccharides were observed in the pool that ranged from 3 pentose to 7 pentose in length. The most abundant structures represent linear α-1,4 glucose polymers (3Hex, 4.11 min; 4Hex 9.29 min; 5Hex 12.31 min; 6Hex, 14.058; 7Hex, 15.254 min; 8Hex, 16.394; 9Hex, 18.013 min; 10Hex, 21.99 min; 11Hex, 22.911; 12Hex, 24.55 min). Other isomers were found and would represent structures with at minimum 1 α-1,6 branch. The full list of oligosaccharide peaks and abundances are found in Table 6. The oligosaccharide pool can be further distinguished by its 1H-13C 2D-NMR (HSQC) fingerprint (FIG. 13A). Prominent peaks include those described in Table 5A.

TABLE 6
Oligosaccharides generated from the COG depolymerization
of amylopectin. Hex refers to hexose sugars, Pent
refers to pentose sugars, HexA refers to hexuronic
acid sugars, and Deoxyhex refers to deoxyhexose sugars.
Hexose sugars comprise solely glucose.

| | | | RT | Oligo | Retention |
|---|---|---|---|---|---|
| Compound | Identity | Mass | (min) | Wt % | Factor |
| 1 | 3Hex | 504.19 | 4.11 | 10.54 | 1.00 |
| 2 | 4Hex | 666.24 | 6.232 | 0.37 | 1.52 |
| 3 | 4Hex | 666.24 | 6.595 | 0.49 | 1.61 |
| 4 | 4Hex | 666.24 | 7.475 | 0.48 | 1.82 |
| 5 | 4Hex | 666.24 | 8.216 | 0.59 | 2.00 |
| 6 | 4Hex | 666.24 | 8.663 | 0.28 | 2.11 |
| 7 | 4Hex | 666.24 | 9.29 | 23.07 | 2.26 |
| 8 | 5Hex | 828.29 | 9.85 | 0.46 | 2.40 |
| 9 | 5Hex | 828.29 | 10.155 | 0.33 | 2.47 |
| 10 | 5Hex | 828.29 | 10.58 | 0.91 | 2.57 |
| 11 | 5Hex | 828.29 | 11.437 | 0.48 | 2.78 |
| 12 | 5Hex | 828.29 | 12.311 | 25.53 | 3.00 |
| 13 | 6Hex | 990.34 | 12.538 | 0.26 | 3.05 |
| 14 | 6Hex | 990.34 | 12.917 | 0.99 | 3.14 |
| 15 | 6Hex | 990.34 | 13.441 | 0.65 | 3.27 |
| 16 | 6Hex | 990.35 | 14.058 | 16.07 | 3.42 |
| 17 | 7Hex | 1152.40 | 15.254 | 9.04 | 3.71 |
| 18 | 8Hex | 1314.45 | 16.394 | 3.44 | 3.99 |
| 19 | 7Hex | 1152.40 | 16.902 | 0.11 | 4.11 |
| 20 | 6Hex | 990.35 | 17.064 | 0.22 | 4.15 |
| 21 | 9Hex | 1476.50 | 17.529 | 0.07 | 4.27 |
| 22 | 9Hex | 1476.50 | 18.013 | 1.38 | 4.38 |
| 23 | 10Hex | 1638.56 | 19.224 | 0.22 | 4.68 |
| 24 | 10Hex | 1638.56 | 20.3 | 1.04 | 4.94 |
| 25 | 12Hex | 1962.66 | 21.991 | 0.54 | 5.35 |
| 26 | 11Hex | 1800.61 | 22.911 | 1.21 | 5.57 |
| 27 | 10Hex | 1638.55 | 23.898 | 0.06 | 5.82 |
| 28 | 12Hex | 1962.66 | 24.551 | 1.09 | 5.97 |
| 29 | 12Hex | 1962.66 | 25.186 | 0.08 | 6.13 |

Arabinoxylan refers to a polysaccharide with β1,4xylose backbone with α-1,3 and α-1,2 arabinose branches in a 1 to 2 ratio. The produced oligosaccharides possessed various structural features similar to those in the parent polysaccharide. The xylose composition was 60.28% followed by 36.99% arabinose and 2.08% galactose (Table 3A). With the glycosidic linkage composition being 30.55% terminal-arabinose, 31.20% 4 linked xylose, 22.22% 3,4 linked xylose and 2.65% terminal xylose (Table 4A). 22 oligosaccharides were observed in the pool that ranged from 3 pentose to 7 pentose in length. The most abundant structures represent 3pent, 8.612 min and 14.346 min; 4pent, 20.455 min; Spent, 20.812 min and 25.947 min; 6 pent, 24.969 min; 7 pent, 27.697 min; The full list of oligosaccharide peaks and abundances are found in Table 7. The oligosaccharide pool can be further distinguished by it's 1H-13C 2D-NMR (HSQC) fingerprint (FIG. 13A). Prominent peaks include those described in Table 5A.

TABLE 7
Oligosaccharides generated from the COG depolymerization
of Arabinoxylan. Hex refers to hexose sugars, Pent
refers to pentose sugars, HexA refers to hexuronic
acid sugars, and Deoxyhex refers to deoxyhexose sugars.
Pentose sugars refer to arabinose and xylose.

| | | | RT | Oligo | Retention |
|---|---|---|---|---|---|
| Compound | Identity | Mass | (min) | Wt % | Factor |
| 1 | 3Pent | 414.15 | 5.215 | 2.15 | 1 |
| 2 | 3Pent | 414.15 | 8.612 | 5.60 | 1.651 |
| 3 | 3Pent | 414.15 | 11.556 | 5.01 | 2.216 |
| 4 | 3Pent | 414.15 | 14.346 | 8.99 | 2.751 |
| 5 | 3Pent | 414.15 | 21.696 | 3.32 | 4.16 |
| 6 | 4Pent | 546.2 | 7.539 | 2.29 | 1.446 |
| 7 | 4Pent | 546.2 | 8.925 | 5.02 | 1.711 |
| 8 | 4Pent | 546.19 | 16.952 | 4.99 | 3.251 |
| 9 | 4Pent | 546.2 | 20.455 | 5.68 | 3.922 |
| 10 | 5Pent | 678.24 | 11.806 | 3.97 | 2.264 |
| 11 | 5Pent | 678.24 | 16.229 | 4.00 | 3.112 |
| 12 | 5Pent | 678.24 | 19.91 | 5.20 | 3.818 |
| 13 | 5Pent | 678.24 | 20.812 | 8.23 | 3.991 |
| 14 | 5Pent | 678.24 | 23.691 | 3.63 | 4.543 |
| 15 | 5Pent | 678.24 | 25.947 | 6.89 | 4.975 |
| 16 | 6Pent | 810.28 | 17.177 | 2.16 | 3.294 |
| 17 | 6Pent | 810.28 | 21.326 | 2.88 | 4.089 |
| 18 | 6Pent | 810.28 | 24.969 | 4.62 | 4.788 |
| 19 | 6Pent | 810.28 | 25.26 | 3.51 | 4.844 |
| 20 | 6Pent | 810.28 | 26.913 | 3.30 | 5.161 |
| 21 | 7Pent | 942.32 | 25.618 | 2.90 | 4.912 |
| 22 | 7Pent | 942.32 | 27.697 | 5.66 | 5.311 |

Xyloglucan refers to a polysaccharide with β-1,4 glucose backbone with α-1,6xylose branches. In a 1 to 2 ratio branches may be further extended via the addition of β-2,1 galactose. The produced oligosaccharides possessed various structural features similar to those in the parent polysaccharide. The glucose composition was 48.75% followed by 36.99% xylose and 14.14% galactose (Table 3A). With the glycosidic linkage composition being 4-glucose, 4,6 glucose, 6 glucose, and terminal glucose at 28.23%, 20.49%, 5.63% and 4.23% respectively, with terminal-galactose being 20.62% (Table 4A). In addition, further linkages were seen as terminal-xylan 10.78% and 2-xylan 5.81% (Table 4A). 42 oligosaccharides were observed in the pool that ranged from 2Hex1Pent to 5Hex3Pent in length. The most abundant structures represent 2Hex1Pent, 6.596 min; 2Hex2Pent, 14.055 min; 3Hex1Pent, 12.735; 3Hex2Pent, 23.712 min and 22.6 min; 4Hex2Pent 24.966 min and 29.18 min; 4Hex3Pent 26.017. The full list of oligosaccharide peaks and abundances are found in Table 8. The oligosaccharide pool can be further distinguished by its 1H-13C 2D-NMR (HSQC) fingerprint (FIG. 13A). Prominent peaks include those described in Table 5A.

TABLE 8
Oligosaccharides generated from the COG depolymerization of
Xyloglucan. Hex refers to hexose sugars, Pent refers to pentose
sugars, HexA refers to hexuronic acid sugars, and Deoxyhex
refers to deoxyhexose sugars. Hexose sugars refer to glucose
and galactose. Pentose sugars refer to xylose.

| | | | RT | Oligo | Retention |
|---|---|---|---|---|---|
| Compound | Identity | Mass | (min) | Wt % | Factor |
| 1 | 2Hex1Pent | 474.17 | 6.596 | 8.71 | 1 |
| 2 | 2Hex1Pent | 474.17 | 7.182 | 1.54 | 1.089 |
| 3 | 2Hex1Pent | 474.17 | 9.915 | 6.7 | 1.503 |
| 4 | 2Hex2Pent | 606.22 | 10.489 | 0.41 | 1.59 |
| 5 | 2Hex2Pent | 606.22 | 14.055 | 8.77 | 2.131 |
| 6 | 3Hex1Pent | 636.23 | 11.569 | 2.3 | 1.754 |
| 7 | 3Hex1Pent | 636.23 | 12.735 | 4.64 | 1.931 |
| 8 | 3Hex1Pent | 636.23 | 17.858 | 2.32 | 2.707 |
| 9 | 3Hex1Pent | 636.23 | 19.639 | 2.6 | 2.977 |
| 10 | 3Hex1Pent | 636.23 | 21.047 | 2.57 | 3.191 |
| 11 | 3Hex2Pent | 768.27 | 16.236 | 1.77 | 2.461 |
| 12 | 3Hex2Pent | 768.27 | 16.913 | 7.56 | 2.564 |
| 13 | 3Hex2Pent | 768.27 | 22.6 | 4.71 | 3.426 |
| 14 | 3Hex2Pent | 768.27 | 23.712 | 4.79 | 3.595 |
| 15 | 3Hex2Pent | 768.27 | 24.427 | 2.85 | 3.703 |
| 16 | 3Hex3Pent | 900.31 | 25.675 | 1.94 | 3.893 |
| 17 | 4Hex1Pent | 798.28 | 19.976 | 2.44 | 3.029 |
| 18 | 4Hex1Pent | 798.28 | 22.29 | 1.57 | 3.379 |
| 19 | 4Hex1Pent | 798.28 | 26.972 | 0.62 | 4.089 |
| 20 | 4Hex1Pent | 798.28 | 27.808 | 0.91 | 4.216 |
| 21 | 4Hex1Pent | 798.28 | 28.061 | 0.79 | 4.254 |
| 22 | 4Hex2Pent | 930.32 | 18.582 | 2.65 | 2.817 |
| 23 | 4Hex2Pent | 930.32 | 22.74 | 1.7 | 3.448 |
| 24 | 4Hex2Pent | 930.32 | 23.905 | 1.69 | 3.624 |
| 25 | 4Hex2Pent | 930.32 | 24.564 | 3.31 | 3.724 |
| 26 | 4Hex2Pent | 930.32 | 24.966 | 3.86 | 3.785 |
| 27 | 4Hex2Pent | 930.32 | 27.736 | 1.18 | 4.205 |
| 28 | 4Hex2Pent | 930.32 | 28.44 | 0.98 | 4.312 |
| 29 | 4Hex2Pent | 930.32 | 28.778 | 1.97 | 4.363 |
| 30 | 4Hex2Pent | 930.32 | 29.18 | 1.24 | 4.424 |
| 31 | 4Hex2Pent | 930.32 | 30.836 | 0.75 | 4.675 |
| 32 | 4Hex3Pent | 1062.36 | 26.017 | 1.49 | 3.944 |
| 33 | 4Hex3Pent | 1062.36 | 26.225 | 1.2 | 3.976 |
| 34 | 4Hex3Pent | 1062.36 | 29.365 | 0.99 | 4.452 |
| 35 | 4Hex3Pent | 1062.36 | 29.76 | 0.96 | 4.512 |
| 36 | 4Hex3Pent | 1062.36 | 31.172 | 0.58 | 4.726 |
| 37 | 4Hex3Pent | 1062.36 | 31.49 | 1.02 | 4.774 |
| 38 | 5Hex3Pent | 1224.42 | 26.484 | 1.27 | 4.015 |
| 39 | 5Hex3Pent | 1224.42 | 28.91 | 0.54 | 4.383 |
| 40 | 5Hex3Pent | 1224.42 | 29.282 | 0.74 | 4.439 |
| 41 | 5Hex3Pent | 1224.42 | 30.009 | 0.89 | 4.55 |
| 42 | 5Hex3Pent | 1224.42 | 31.349 | 0.47 | 4.753 |

B-Glucan refers to a polysaccharide with a β-1,4 β-1,3 in a 4 to 1 ratio glucose backbone. The produced oligosaccharides possessed various structural features similar to those in the parent polysaccharide. The glucose composition was 97.04% (Table 3A). With the glycosidic linkage composition being 4-glucose, 3 glucose, and terminal glucose at 48.91%, 30.95%, and 17.06% respectively (Table 4A). 15 oligosaccharides were observed in the pool that ranged from 3 hexoses to 6 hexoses in length. The most abundant structures represent 3Hex, 14.158 min; 4Hex 9.81 min and 11.27 min 5Hex 7.33 min and 11.24 min; 6Hex, 34.032 min. The full list of oligosaccharide peaks and abundances are found in Table 9. The oligosaccharide pool can be further distinguished by its 1H-13C 2D-NMR (HSQC). Prominent peaks include those described in Table 5A.

TABLE 9
Oligosaccharides generated from the COG depolymerization of β-glucan.
Hex refers to hexose sugars, Pent refers to pentose sugars, HexA
refers to hexuronic acid sugars, and Deoxyhex refers to deoxyhexose
sugars. Hexose sugars refer solely to glucose.

| | | | RT | Oligo | Retention |
|---|---|---|---|---|---|
| Compound | Identity | Mass | (min) | Wt % | Factor |
| 1 | 3Hex | 504.19 | 10.431 | 7.9 | 1 |
| 2 | 3Hex | 504.19 | 14.158 | 13.53 | 1.357 |
| 3 | 3Hex | 504.19 | 17.074 | 5.47 | 1.637 |
| 4 | 4Hex | 666.24 | 21.248 | 11.27 | 2.037 |
| 5 | 4Hex | 666.24 | 24.78 | 6.29 | 2.376 |
| 6 | 4Hex | 666.24 | 25.028 | 8.82 | 2.399 |
| 7 | 4Hex | 666.24 | 25.857 | 9.81 | 2.479 |
| 8 | 5Hex | 828.29 | 27.544 | 2.08 | 2.641 |
| 9 | 5Hex | 828.29 | 28.172 | 3.8 | 2.701 |
| 10 | 5Hex | 828.29 | 28.914 | 1.18 | 2.772 |
| 11 | 5Hex | 828.29 | 29.507 | 7.33 | 2.829 |
| 12 | 5Hex | 828.29 | 30.241 | 11.24 | 2.899 |
| 13 | 6Hex | 990.34 | 32.369 | 2.62 | 3.103 |
| 14 | 6Hex | 990.34 | 34.032 | 6.74 | 3.263 |
| 15 | 6Hex | 990.34 | 37.05 | 1.95 | 3.552 |

Galactomnannan refers to a polysaccharide with a β-1,4 mannose backbone, with 22% α-1,3 galactose branching. The produced oligosaccharides possessed various structural features similar to those in the parent polysaccharide. The mannose composition being 78.14% and galactose being 18.91% (Table 3A). With the glycosidic linkage composition being 4-mannose, terminal mannose and 4,6-mannose at 47.34%, 20.76%, and 6.52% respectively, with terminal-galactose being 17.85% and 2.34% 4-glucose (Table 4A). 54 oligosaccharides were observed in the pool that ranged from 3 hexose to 7 hexoses in length. The most abundant structures represent 3Hex, 1.489 min; 4Hex 4.109 min and 5.122 min; 4Hex1HexA, 10.301 min; 4Hex1Pent, 9.614 min 5Hex 7.65 min; 6Hex, 11.077 min; 7Hex, 13.245 min. The full list of oligosaccharide peaks and abundances are found in Table 10. The oligosaccharide pool can be further distinguished by its 1H-13C 2D-NMR (HSQC) fingerprint (FIG. 13A). Prominent peaks include those described in Table 5A.

TABLE 10
Oligosaccharides generated from the COG depolymerization
of Galactomannan. Hex refers to hexose sugars, Pent
refers to pentose sugars, HexA refers to hexuronic
acid sugars, and Deoxyhex refers to deoxyhexose sugars.
Hexoses refer to galactose and mannose.

| | | | RT | Oligo | Retention |
|---|---|---|---|---|---|
| Compound | Identity | Mass | (min) | Wt % | Factor |
| 1 | 3Hex | 504.19 | 1.489 | 2.72 | 1 |
| 2 | 3Hex | 504.19 | 1.845 | 1.04 | 1.2391 |
| 3 | 3Hex | 504.19 | 2.978 | 1.54 | 2 |
| 4 | 3Hex1Pent | 636.23 | 6.404 | 1.79 | 4.3009 |
| 5 | 3Hex1Pent | 636.23 | 6.968 | 1.16 | 4.6797 |
| 6 | 3Hex1Pent | 636.23 | 8.198 | 0.38 | 5.5057 |
| 7 | 3Hex1Pent | 636.23 | 8.566 | 0.55 | 5.7529 |
| 8 | 3Hex2Pent | 768.27 | 10.827 | 0.28 | 7.2713 |
| 9 | 3Hex2Pent | 768.27 | 12.342 | 0.23 | 8.2888 |
| 10 | 3Hex2Pent | 768.27 | 13.372 | 0.43 | 8.9805 |
| 11 | 4Hex | 666.24 | 4.109 | 12.41 | 2.7596 |
| 12 | 4Hex | 666.24 | 4.802 | 4.74 | 3.225 |
| 13 | 4Hex | 666.24 | 5.122 | 5.89 | 3.4399 |
| 14 | 4Hex | 666.24 | 5.502 | 6.4 | 3.6951 |
| 15 | 4Hex | 666.24 | 7.239 | 3.54 | 4.8617 |
| 16 | 4Hex1Deoxyhex | 812.26 | 10.495 | 0.58 | 7.0484 |
| 17 | 4Hex1Deoxyhex | 812.26 | 14.627 | 0.42 | 9.8234 |
| 18 | 4Hex1HexA | 842.27 | 9.515 | 0.39 | 6.3902 |
| 19 | 4Hex1HexA | 842.27 | 10.301 | 1.26 | 6.9181 |
| 20 | 4Hex1HexA | 842.27 | 11.02 | 0.71 | 7.4009 |
| 21 | 4Hex1HexA | 842.27 | 11.725 | 0.52 | 7.8744 |
| 22 | 4Hex1HexA | 842.27 | 12.33 | 0.98 | 8.2807 |
| 23 | 4Hex1HexA | 842.27 | 14.866 | 0.43 | 9.9839 |
| 24 | 4Hex1HexA | 842.27 | 16.706 | 0.33 | 11.2196 |
| 25 | 4Hex1Pent | 798.28 | 9.614 | 1.3 | 6.4567 |
| 26 | 4Hex1Pent | 798.28 | 10.996 | 0.83 | 7.3848 |
| 27 | 4Hex1Pent | 798.28 | 11.525 | 0.31 | 7.7401 |
| 28 | 4Hex1Pent | 798.28 | 14.191 | 0.44 | 9.5306 |
| 29 | 5Hex | 828.29 | 7.65 | 7.46 | 5.1377 |
| 30 | 5Hex | 828.29 | 7.896 | 3.61 | 5.3029 |
| 31 | 5Hex | 828.29 | 8.52 | 2.77 | 5.722 |
| 32 | 5Hex | 828.29 | 9.013 | 3.16 | 6.0531 |
| 33 | 5Hex | 828.29 | 9.737 | 2.53 | 6.5393 |
| 34 | 5Hex | 828.29 | 9.999 | 4.5 | 6.7152 |
| 35 | 5Hex | 828.29 | 11.477 | 0.62 | 7.7079 |
| 36 | 5Hex | 828.29 | 13.038 | 3.41 | 8.7562 |
| 37 | 6Hex | 990.34 | 11.077 | 3.58 | 7.4392 |
| 38 | 6Hex | 990.34 | 11.342 | 1.11 | 7.6172 |
| 39 | 6Hex | 990.34 | 11.581 | 1.58 | 7.7777 |
| 40 | 6Hex | 990.34 | 11.96 | 1.27 | 8.0322 |
| 41 | 6Hex | 990.34 | 12.288 | 1.28 | 8.2525 |
| 42 | 6Hex | 990.34 | 12.746 | 0.79 | 8.5601 |
| 43 | 6Hex | 990.34 | 13.842 | 1.74 | 9.2962 |
| 44 | 6Hex | 990.34 | 14.332 | 2.32 | 9.6253 |
| 45 | 6Hex | 990.34 | 15.227 | 1.21 | 10.2263 |
| 46 | 6Hex | 990.34 | 17.443 | 0.31 | 11.7146 |
| 47 | 7Hex | 1152.4 | 13.245 | 1.35 | 8.8952 |
| 48 | 7Hex | 1152.4 | 14.057 | 0.86 | 9.4406 |
| 49 | 7Hex | 1152.39 | 14.302 | 0.48 | 9.6051 |
| 50 | 7Hex | 1152.39 | 14.551 | 0.39 | 9.7723 |
| 51 | 7Hex | 1152.4 | 15.271 | 0.49 | 10.2559 |
| 52 | 7Hex | 1152.39 | 15.516 | 0.31 | 10.4204 |
| 53 | 7Hex | 1152.4 | 16.316 | 0.79 | 10.9577 |
| 54 | 7Hex | 1152.39 | 17.823 | 0.51 | 11.9698 |

Arabinogalactan II refers to a polysaccharide with a β-1,3 galactose backbone, extensive branching comprising of α-1,6 arabinose, β-1,6 galactose-β-1,6 galactose, β-1,6 galactose-α-1,4 arabinose and β-1,4 galactose-β-1,6 galactose. The produced oligosaccharides possessed various structural features similar to those in the parent polysaccharide. The galactose composition being 87.28% and arabinose being 7.23% (Table 3A). With the glycosidic linkage composition being terminal galactose, 1,3 galactose, 1,3,6 galactose and 6 galactose at 50.75%, 17.33%, 14.18%, and 11.83% respectively, with terminal arabinose being 3.28% (Table 4A). 62 oligosaccharides were observed in the pool that ranged from 3 hexose to 6 hexoses in length. The most abundant structures represent 3Hex, 2.53 min and 5.552 min; 4Hex 3.534 min and 8.843 min; 5Hex 10.555 min and 11.7 min 6Hex, 12.269. The full list of oligosaccharide peaks and abundances are found in Table 11. The oligosaccharide pool can be further distinguished by its 1H-13C 2D-NMR (HSQC) fingerprint (FIG. 13A). Prominent peaks include those described in Table 5A.

TABLE 11
Oligosaccharides generated from the COG depolymerization
of Arabinogalactan II. Hex refers to hexose sugars, Pent
refers to pentose sugars, HexA refers to hexuronic acid
sugars, and Deoxyhex refers to deoxyhexose sugars. Pentoses
refer to arabinose and hexoses refer to galactose.

| | | | RT | Oligo | Retention |
|---|---|---|---|---|---|
| Compound | Identity | Mass | (min) | Wt % | Factor |
| 1 | 2Hex | 342.13 | 1.233 | 0.58 | 1 |
| 2 | 2Hex | 342.13 | 1.43 | 1.79 | 1.16 |
| 3 | 2Hex1Pent | 474.18 | 2.505 | 0.47 | 2.032 |
| 4 | 2Hex1Pent | 474.18 | 3.087 | 0.77 | 2.504 |
| 5 | 2Hex1Pent | 474.18 | 5.487 | 0.38 | 4.45 |
| 6 | 2Hex1Pent | 474.18 | 5.912 | 0.19 | 4.795 |
| 7 | 2Hex1Pent | 474.18 | 6.635 | 0.18 | 5.381 |
| 8 | 2Hex1Pent | 474.18 | 9.208 | 0.61 | 7.468 |
| 9 | 2Hex1Pent | 474.18 | 9.452 | 0.28 | 7.666 |
| 10 | 2Hex2Pent | 606.22 | 3.233 | 0.29 | 2.622 |
| 11 | 2Hex2Pent | 606.22 | 4.847 | 0.75 | 3.931 |
| 12 | 2Hex2Pent | 606.22 | 7.968 | 0.27 | 6.462 |
| 13 | 3Hex | 504.19 | 1.679 | 1.59 | 1.362 |
| 14 | 3Hex | 504.19 | 2.53 | 3.99 | 2.052 |
| 15 | 3Hex | 504.19 | 2.732 | 1.91 | 2.216 |
| 16 | 3Hex | 504.19 | 5.552 | 4.44 | 4.503 |
| 17 | 3Hex1Pent | 636.23 | 6.836 | 0.85 | 5.544 |
| 18 | 3Hex1Pent | 636.23 | 8.019 | 0.25 | 6.504 |
| 19 | 3Hex1Pent | 636.23 | 9.729 | 0.23 | 7.891 |
| 20 | 3Hex1Pent | 636.23 | 9.949 | 0.63 | 8.069 |
| 21 | 3Hex1Pent | 636.23 | 11.439 | 1.27 | 9.277 |
| 22 | 3Hex2Pent | 768.27 | 8.577 | 0.20 | 6.956 |
| 23 | 3Hex3Pent | 900.31 | 12.393 | 0.29 | 10.051 |
| 24 | 4Hex | 666.24 | 3.534 | 6.47 | 2.866 |
| 25 | 4Hex | 666.24 | 4.2 | 5.10 | 3.406 |
| 26 | 4Hex | 666.24 | 5.381 | 4.02 | 4.364 |
| 27 | 4Hex | 666.24 | 6.643 | 5.20 | 5.388 |
| 28 | 4Hex | 666.24 | 8.843 | 7.15 | 7.172 |
| 29 | 4Hex | 666.24 | 9.955 | 2.70 | 8.074 |
| 30 | 4Hex | 666.24 | 11.036 | 0.91 | 8.951 |
| 31 | 4Hex | 666.24 | 14.586 | 1.15 | 11.83 |
| 32 | 4Hex1Pent | 798.28 | 12.257 | 0.21 | 9.941 |
| 33 | 4Hex1Pent | 798.28 | 12.449 | 0.23 | 10.097 |
| 34 | 4Hex1Pent | 798.28 | 12.783 | 0.63 | 10.367 |
| 35 | 4Hex1Pent | 798.28 | 14.336 | 0.50 | 11.627 |
| 36 | 4Hex1Pent | 798.28 | 18.353 | 0.52 | 14.885 |
| 37 | 5Hex | 828.29 | 6.433 | 2.57 | 5.217 |
| 38 | 5Hex | 828.29 | 6.983 | 2.91 | 5.663 |
| 39 | 5Hex | 828.29 | 9.107 | 2.45 | 7.386 |
| 40 | 5Hex | 828.29 | 10.555 | 4.78 | 8.56 |
| 41 | 5Hex | 828.29 | 11.023 | 2.10 | 8.94 |
| 42 | 5Hex | 828.29 | 11.7 | 5.43 | 9.489 |
| 43 | 5Hex | 828.29 | 14.088 | 0.77 | 11.426 |
| 44 | 5Hex | 828.29 | 15.047 | 3.80 | 12.204 |
| 45 | 6Hex | 990.34 | 8.723 | 0.94 | 7.075 |
| 46 | 6Hex | 990.34 | 11.052 | 0.76 | 8.964 |
| 47 | 6Hex | 990.35 | 11.731 | 1.68 | 9.514 |
| 48 | 6Hex | 990.34 | 11.938 | 1.53 | 9.682 |
| 49 | 6Hex | 990.34 | 12.269 | 2.86 | 9.951 |
| 50 | 6Hex | 990.34 | 14.213 | 0.73 | 11.527 |
| 51 | 6Hex | 990.35 | 14.432 | 0.24 | 11.705 |
| 52 | 6Hex | 990.35 | 15.11 | 1.94 | 12.255 |
| 53 | 6Hex | 990.34 | 16.676 | 1.33 | 13.525 |
| 54 | 6Hex | 990.35 | 17.193 | 0.92 | 13.944 |
| 55 | 6Hex | 990.34 | 17.84 | 0.56 | 14.469 |
| 56 | 6Hex | 990.34 | 21.269 | 1.03 | 17.25 |
| 57 | 7Hex | 1152.4 | 12.943 | 0.51 | 10.497 |
| 58 | 7Hex | 1152.39 | 13.193 | 0.47 | 10.7 |
| 59 | 7Hex | 1152.39 | 13.639 | 0.53 | 11.062 |
| 60 | 7Hex | 1152.4 | 16.038 | 0.26 | 13.007 |
| 61 | 7Hex | 1152.4 | 16.731 | 0.50 | 13.569 |
| 62 | 7Hex | 1152.4 | 17.67 | 1.40 | 14.331 |

Curdlan refers to a polysaccharide with a β-1,3 glucose backbone. The produced oligosaccharides possessed various structural features similar to those in the parent polysaccharide. The glucose composition being 99.04% (Table 3A). With the glycosidic linkage composition being 1,3 glucose at 74.84% and 8.82% being terminal glucose (Table 4A). 10 oligosaccharides were observed in the pool that ranged from 2 hexose to 6 hexoses in length. The most abundant structures represent 2Hex, 1.456 min, 3Hex, 1.456 min; 2Hex1Pent, 12.672 min; 4Hex 24.35 min; 5Hex 30.063 min; 6Hex, 36.833. The full list of oligosaccharide peaks and abundances are found in Table 12. The oligosaccharide pool can be further distinguished by its 1H-13C 2D-NMR (HSQC). Prominent peaks include those described in Table 5A.

TABLE 12
Oligosaccharides generated from the COG depolymerization of
Curdlan. Hex refers to hexose sugars, Pent refers to pentose
sugars, HexA refers to hexuronic acid sugars, and Deoxyhex
refers to deoxyhexose sugars. Hexoses refer to glucose.

| | | | RT | Oligo | Retention |
|---|---|---|---|---|---|
| Compound | Identity | Mass | (min) | Wt % | Factor |
| 1 | 1Hex1Pent | 312.12 | 1.663 | 0.75 | 1.14 |
| 2 | 2Hex | 342.13 | 1.456 | 3.34 | 1.00 |
| 3 | 2Hex1Pent | 474.17 | 12.672 | 4.45 | 8.70 |
| 4 | 2Hex1Pent | 474.17 | 13.644 | 1.57 | 9.37 |
| 5 | 3Hex | 504.18 | 7.423 | 0.85 | 5.10 |
| 6 | 3Hex | 504.19 | 11.902 | 38.62 | 8.17 |
| 7 | 3Hex1Pent | 636.23 | 24.761 | 0.98 | 17.01 |
| 8 | 4Hex | 666.24 | 24.35 | 20.24 | 16.72 |
| 9 | 5Hex | 828.29 | 30.063 | 18.87 | 20.65 |
| 10 | 6Hex | 990.34 | 36.833 | 10.32 | 25.30 |

Lichenan refers to a polysaccharide with a β-1,4 glucose backbone with alternating β-1,3 glucose 33% of the time. The produced oligosaccharides possessed various structural features similar to those in the parent polysaccharide. The glucose composition being 80.21%, with galactose and mannose both being 8.64% (Table 3A). With the glycosidic linkage composition being 4-mannose, 4,6-mannose and terminal mannose at 67.02%, 8.95%, and 6.82% respectively, with terminal-galactose being 19.58% (Table 4A). 42 oligosaccharides were observed in the pool that ranged from 3 hexose to 8 hexoses in length. The most abundant structures represent 3Hex1Pent, 7.59 min; 4Hex 17.74 min; 4Hex1Pent, 6.96 min; Hex 15.88 min; Hex1HexA, 12.747 min, 6Hex, 10.877 min; 7Hex, 13.039 min. The full list of oligosaccharide peaks and abundances are found in Table 13. The oligosaccharide pool can be further distinguished by its 1H-13C 2D-NMR (HSQC) fingerprint (FIG. 13A). Prominent peaks include those described in Table 5A.

TABLE 13
Oligosaccharides generated from the COG depolymerization of
Lichenan. Hex refers to hexose sugars, Pent refers to pentose
sugars, HexA refers to hexuronic acid sugars, and Deoxyhex
refers to deoxyhexose sugars. Hexoses refer to glucose.

| | | | RT | Oligo | Retention |
|---|---|---|---|---|---|
| Compound | Identity | Mass | (min) | Wt % | Factor |
| 1 | 1Hex2Pent | 444.17 | 2.988 | 0.50 | 2.008 |
| 2 | 2Hex | 342.13 | 2.925 | 0.46 | 1.966 |
| 3 | 2Hex1Pent | 474.18 | 2.211 | 0.62 | 1.486 |
| 4 | 2Hex2Pent | 606.22 | 8.398 | 2.08 | 5.644 |
| 5 | 3Hex | 504.19 | 1.488 | 0.99 | 1 |
| 6 | 3Hex | 504.18 | 6.593 | 0.81 | 4.431 |
| 7 | 3Hex1Deoxyhex | 650.24 | 5.754 | 0.30 | 3.867 |
| 8 | 3Hex1HexA | 680.22 | 5.977 | 1.65 | 4.017 |
| 9 | 3Hex1Pent | 636.23 | 6.275 | 7.59 | 4.217 |
| 10 | 3Hex1Pent | 636.23 | 6.83 | 1.29 | 4.59 |
| 11 | 3Hex1Pent | 636.23 | 8.396 | 0.29 | 5.642 |
| 12 | 3Hex2Pent | 768.27 | 10.68 | 1.43 | 7.177 |
| 13 | 4Hex | 666.24 | 4.016 | 17.74 | 2.699 |
| 14 | 4Hex | 666.24 | 4.726 | 0.99 | 3.176 |
| 15 | 4Hex | 666.24 | 5.06 | 0.58 | 3.401 |
| 16 | 4Hex | 666.24 | 5.439 | 0.93 | 3.655 |
| 17 | 4Hex | 666.24 | 9.279 | 0.88 | 6.236 |
| 18 | 4Hex | 666.24 | 10.087 | 0.35 | 6.779 |
| 19 | 4Hex | 666.24 | 16.429 | 1.54 | 11.041 |
| 20 | 4Hex1Deoxyhex | 812.26 | 10.22 | 0.78 | 6.868 |
| 21 | 4Hex1HexA | 842.27 | 9.907 | 4.28 | 6.658 |
| 22 | 4Hex1HexA | 842.27 | 10.504 | 0.59 | 7.059 |
| 23 | 4Hex1Pent | 798.28 | 9.459 | 6.96 | 6.357 |
| 24 | 5Hex | 828.29 | 7.567 | 15.88 | 5.085 |
| 25 | 5Hex | 828.29 | 8.882 | 0.70 | 5.969 |
| 26 | 5Hex | 828.29 | 9.089 | 0.61 | 6.108 |
| 27 | 5Hex | 828.29 | 9.783 | 1.38 | 6.575 |
| 28 | 5Hex | 828.29 | 17.217 | 0.47 | 11.571 |
| 29 | 5Hex | 828.29 | 18.075 | 0.52 | 12.147 |
| 30 | 5Hex1Deoxyhex | 974.31 | 13.623 | 0.88 | 9.155 |
| 31 | 5Hex1HexA | 1004.33 | 12.747 | 4.44 | 8.567 |
| 32 | 5Hex1HexA | 1004.32 | 13.358 | 0.79 | 8.977 |
| 33 | 5Hex1Pent | 960.34 | 12.139 | 2.06 | 8.158 |
| 34 | 6Hex | 990.35 | 10.877 | 8.71 | 7.31 |
| 35 | 6Hex | 990.35 | 11.152 | 0.34 | 7.495 |
| 36 | 6Hex | 990.34 | 11.405 | 0.55 | 7.665 |
| 37 | 6Hex | 990.35 | 11.76 | 0.64 | 7.903 |
| 38 | 6Hex | 990.34 | 14.094 | 0.59 | 9.472 |
| 39 | 6Hex1HexA | 1166.38 | 15.181 | 1.68 | 10.202 |
| 40 | 6Hex1Pent | 1122.39 | 14.439 | 0.96 | 9.704 |
| 41 | 7Hex | 1152.4 | 13.039 | 4.18 | 8.763 |
| 42 | 8Hex | 1314.45 | 14.906 | 0.98 | 10.017 |

Mannan refers to a polysaccharide with a β-1,4 mannose backbone. The produced oligosaccharides possessed various structural features similar to those in the parent polysaccharide. The mannose composition being 83.8%, followed by galactose, glucose, and arabinose at 7.61%, 4.48% and 2.99% respectively (Table 3A). With the glycosidic linkage composition being 4-mannose, and terminal mannose 58.31%, and 34.6% respectively, with terminal-galactose being 3.63% (Table 4A). 46 oligosaccharides were observed in the pool that ranged from 1 hexose and 1 pentose to hexoses and 2 pentoses in length. The most abundant structures represent 2Hex1Pent, 6.624 min and 9.655 min; 2Hex1Pent, 13.77 min; 3Hex1Pent 12.727 min; 3Hex2Pent 16.706 min and 23.412 min; 4Hex1pent, 19.731 min; 4Hex2pent, 24.422 min. The full list of oligosaccharide peaks and abundances are found in Table 14. The full list of oligosaccharides can be further distinguished by its 1H-13C 2D-NMR (HSQC). Prominent peaks include those described in Table 5A.

TABLE 14
Oligosaccharides generated from the COG depolymerization of
Mannan. Hex refers to hexose sugars, Pent refers to pentose
sugars, HexA refers to hexuronic acid sugars, and Deoxyhex
refers to deoxyhexose sugars. Hexoses refer to mannose.

| | | | RT | Oligo | Retention |
|---|---|---|---|---|---|
| Compound | Identity | Mass | (min) | Wt % | Factor |
| 1 | 1Hex1Pent | 312.12 | 1.583 | 1.24 | 1 |
| 2 | 1Hex2Pent | 444.17 | 7.463 | 1.10 | 4.714 |
| 3 | 1Hex2Pent | 444.16 | 7.984 | 0.37 | 5.044 |
| 4 | 2Hex1Pent | 474.18 | 6.624 | 6.54 | 4.184 |
| 5 | 2Hex1Pent | 474.18 | 6.954 | 1.33 | 4.393 |
| 6 | 2Hex1Pent | 474.18 | 9.655 | 7.95 | 6.099 |
| 7 | 2Hex1Pent | 474.18 | 12.409 | 0.24 | 7.839 |
| 8 | 2Hex2Pent | 606.22 | 10.576 | 0.66 | 6.681 |
| 9 | 2Hex2Pent | 606.22 | 13.77 | 8.45 | 8.699 |
| 10 | 2Hex2Pent | 606.22 | 16.102 | 0.47 | 10.172 |
| 11 | 2Hex2Pent | 606.22 | 18.235 | 0.51 | 11.519 |
| 12 | 2Hex2Pent | 606.22 | 19.122 | 0.74 | 12.08 |
| 13 | 3Hex | 504.19 | 13.847 | 1.40 | 8.747 |
| 14 | 3Hex1HexA | 680.22 | 27.032 | 0.84 | 17.076 |
| 15 | 3Hex1HexA | 680.22 | 29.171 | 0.69 | 18.428 |
| 16 | 3Hex1Pent | 636.23 | 11.423 | 2.81 | 7.216 |
| 17 | 3Hex1Pent | 636.23 | 12.727 | 5.63 | 8.04 |
| 18 | 3Hex1Pent | 636.23 | 17.531 | 3.44 | 11.075 |
| 19 | 3Hex1Pent | 636.23 | 19.328 | 3.95 | 12.21 |
| 20 | 3Hex1Pent | 636.23 | 20.683 | 4.59 | 13.066 |
| 21 | 3Hex2Pent | 768.27 | 16.706 | 8.36 | 10.553 |
| 22 | 3Hex2Pent | 768.27 | 22.303 | 3.01 | 14.089 |
| 23 | 3Hex2Pent | 768.27 | 23.412 | 5.81 | 14.79 |
| 24 | 3Hex2Pent | 768.27 | 24.786 | 0.26 | 15.658 |
| 25 | 3Hex2Pent | 768.27 | 25.534 | 0.48 | 16.13 |
| 26 | 4Hex1Deoxyhex | 812.26 | 28.218 | 0.82 | 17.826 |
| 27 | 4Hex1Deoxyhex | 812.26 | 29.164 | 1.19 | 18.423 |
| 28 | 4Hex1Deoxyhex | 812.26 | 30.672 | 1.25 | 19.376 |
| 29 | 4Hex1Pent | 798.28 | 19.731 | 3.68 | 12.464 |
| 30 | 4Hex1Pent | 798.29 | 21.95 | 3.25 | 13.866 |
| 31 | 4Hex1Pent | 798.28 | 25.871 | 1.14 | 16.343 |
| 32 | 4Hex1Pent | 798.28 | 26.802 | 0.73 | 16.931 |
| 33 | 4Hex2Pent | 930.32 | 18.491 | 3.19 | 11.681 |
| 34 | 4Hex2Pent | 930.32 | 22.608 | 0.87 | 14.282 |
| 35 | 4Hex2Pent | 930.32 | 24.422 | 3.83 | 15.428 |
| 36 | 4Hex2Pent | 930.32 | 24.785 | 2.53 | 15.657 |
| 37 | 4Hex2Pent | 930.32 | 27.594 | 0.72 | 17.431 |
| 38 | 4Hex2Pent | 930.32 | 28.643 | 1.53 | 18.094 |
| 39 | 4Hex2Pent | 930.32 | 30.759 | 0.47 | 19.431 |
| 40 | 5Hex1Pent | 960.34 | 26.217 | 0.49 | 16.562 |
| 41 | 5Hex1Pent | 960.33 | 26.435 | 0.23 | 16.699 |
| 42 | 5Hex2Pent | 1092.38 | 24.1 | 0.88 | 15.224 |
| 43 | 5Hex2Pent | 1092.38 | 25.241 | 0.64 | 15.945 |
| 44 | 5Hex2Pent | 1092.38 | 27.782 | 0.26 | 17.55 |
| 45 | 5Hex2Pent | 1092.38 | 28.33 | 0.68 | 17.896 |
| 46 | 5Hex2Pent | 1092.38 | 29.139 | 0.74 | 18.407 |

Xylan refers to a polysaccharide with a β-1,4 xylose backbone with a 13% β-1,2 Glucose-4-OMe. The produced oligosaccharides possessed various structural features similar to those in the parent polysaccharide. The xylose composition being 85.48%, followed by glucose, mannose and galactose at 5.36%, 4.9% and 2.04% respectively (Table 3A). With the glycosidic linkage composition being 1,4 xylose at 54.71%, 1,4 mannose at 15.28%, 1,4 glucose at 13.61%, terminal xylose at 7.18% and terminal glucose at 5.19% (Table 4A). 15 oligosaccharides were observed in the pool that ranged from 2 pentose to 6 hexoses and 1 pentose in length. The most abundant structures represent 3Pent, 8.429 min; 4Pent, 16.521 min; 4Pent1HexAoMe, 21.15 min; 5Pent, 23.199; 6Pent, 26.735; 6Hex1Pent, 18.422 min. The full list of oligosaccharide peaks and abundances are found in Table 15. The oligosaccharide pool can be further distinguished by it's 1H-13C 2D-NMR (HSQC) fingerprint (FIG. 13A). Prominent peaks include those described in Table 5A.

TABLE 15
Oligosaccharides generated from the COG depolymerization
of Xylan. Hex refers to hexose sugars, Pent refers to pentose
sugars, HexA refers to hexuronic acid sugars, and Deoxyhex
refers to deoxyhexose sugars. Pentoses refer to xylose.
1HexAOMe refer to methylated glucuronic acid.

| | | | RT | Oligo | Retention |
|---|---|---|---|---|---|
| Compound | Identity | Mass | (min) | Wt % | Factor |
| 1 | 2Pent | 282.11 | 6.692 | 1.60 | 1 |
| 2 | 3Pent | 414.16 | 8.429 | 14.82 | 1.26 |
| 3 | 3Pent1HexAOMe | 604.2 | 18.034 | 2.79 | 2.695 |
| 4 | 4Pent | 546.2 | 16.521 | 18.68 | 2.469 |
| 5 | 4Pent1HexAOMe | 736.24 | 20.205 | 2.61 | 3.019 |
| 6 | 4Pent1HexAOMe | 736.24 | 21.15 | 6.91 | 3.16 |
| 7 | 4Pent1HexAOMe | 736.24 | 23.844 | 4.67 | 3.563 |
| 8 | 4Pent1HexAOMe | 736.24 | 25.394 | 2.61 | 3.795 |
| 9 | 4Pent1HexAOMe | 736.24 | 26.435 | 2.91 | 3.95 |
| 10 | 5Hex2Pent | 1092.42 | 8.109 | 1.07 | 1.212 |
| 11 | 5Pent | 678.24 | 8.432 | 1.07 | 1.26 |
| 12 | 5Pent | 678.24 | 23.199 | 19.00 | 3.467 |
| 13 | 6Hex1Pent | 1122.35 | 18.442 | 7.75 | 2.756 |
| 14 | 6Pent | 810.28 | 26.735 | 9.99 | 3.995 |
| 15 | 7Pent | 942.32 | 28.974 | 3.51 | 4.33 |

Galactan refers to a polysaccharide with a β-1,4 galactan backbone. The produced oligosaccharides possessed various structural features similar to those in the parent polysaccharide. The galactan composition being 80.06%, followed by Arabinose, Rhamnose and Galacturonic acid at 9.28%, 4.59% and 3.04% respectively (Table 3A). With the glycosidic linkage composition being 4 galactose, and terminal galactose at 61.68%, and 33.73% respectively, and terminal-arabinose being 2.02% (Table 4A). 17 oligosaccharides were observed in the pool that ranged from 3 hexose to 6 hexoses and a hexuronic acid in length. The most abundant structures represent 3Hex, 2.69 min; 2Hex1Pent, 3.038 min; 4Hex 6.614 min; 3Hex1Pent, 7.292; 3Hex1hexA, 8.937 min; 5Hex 9.652 min; 4Hex1Pent, 10.112 min, 6Hex, 11.525 min, 4Hex1HexA, 11.857 min; 5Hex1HexA, 13.573 min. The full list of oligosaccharide peaks and abundances are found in Table 16. The oligosaccharide pool can be further distinguished by it's 1H-13C 2D-NMR (HSQC). Prominent peaks include those described in Table 5A.

TABLE 16
Oligosaccharides generated from the COG depolymerization of
galactan. Hex refers to hexose sugars, Pent refers to pentose
sugars, HexA refers to hexuronic acid sugars, and Deoxyhex
refers to deoxyhexose sugars. Hexose refers to galactose.

| | | | RT | Oligo | Retention |
|---|---|---|---|---|---|
| Compound | Identity | Mass | (min) | Wt % | Factor |
| 1 | 2Hex1Pent | 474.18 | 2.658 | 0.59 | 1 |
| 2 | 2Hex1Pent | 474.17 | 3.038 | 5.45 | 1.143 |
| 3 | 3Hex | 504.19 | 2.69 | 6.74 | 1.012 |
| 4 | 3Hex1HexA | 680.22 | 8.937 | 9.12 | 3.362 |
| 5 | 3Hex1Pent | 636.23 | 6.014 | 0.60 | 2.263 |
| 6 | 3Hex1Pent | 636.23 | 7.292 | 10.79 | 2.743 |
| 7 | 4Hex | 666.24 | 6.614 | 15.53 | 2.488 |
| 8 | 4Hex1Deoxyhex | 812.26 | 13.126 | 3.46 | 4.938 |
| 9 | 4Hex1HexA | 842.27 | 11.857 | 11.47 | 4.461 |
| 10 | 4Hex1Pent | 798.28 | 9.508 | 0.62 | 3.577 |
| 11 | 4Hex1Pent | 798.28 | 10.112 | 7.58 | 3.804 |
| 12 | 5Hex | 828.29 | 9.652 | 9.27 | 3.631 |
| 13 | 5Hex1Deoxyhex | 974.31 | 14.753 | 2.85 | 5.55 |
| 14 | 5Hex1HexA | 1004.32 | 13.573 | 7.20 | 5.106 |
| 15 | 6Hex | 990.34 | 11.515 | 4.50 | 4.332 |
| 16 | 6Hex1HexA | 1166.37 | 14.673 | 3.11 | 5.52 |
| 17 | 7Hex | 1152.39 | 12.782 | 1.15 | 4.809 |

Glucomannan refers to a polysaccharide with a 60% β-1,4 mannose and 40% β-1,4 glucose backbone. The produced oligosaccharides possessed various structural features similar to those in the parent polysaccharide. The mannose composition being 60.45%, followed by glucose at 36.73% (Table 3A). With the glycosidic linkage composition being 4 mannose, and terminal mannose at 47.58%, and 20.23% respectively, and 31.52% being 4-glucose (Table 4A). 87 oligosaccharides were observed in the pool that ranged from 3 hexose to 8 hexoses in length. The most abundant structures represent 3Hex, 6.695 min; 3Hex1Pent, 18.947 min; 4Hex 16.802 min and 17.38 min; 4Hex1Pent, 20.328 min; Hex 18.549 min and 25.896 min; 6Hex, 22.854 min; 7Hex, 24.537 min. The full list of oligosaccharide peaks and abundances are found in Table 17.

TABLE 17
Oligosaccharides generated from the COG depolymerization
of Glucomannan. Hex refers to hexose sugars, Pent
refers to pentose sugars, HexA refers to hexuronic
acid sugars, and Deoxyhex refers to deoxyhexose
sugars. Hexoses refer to glucose and mannose.

| | | | RT | Oligo | Retention |
|---|---|---|---|---|---|
| Compound | Identity | Mass | (min) | Wt % | Factor |
| 1 | 2Hex1Pent | 474.17 | 2.263 | 0.17 | 1.518 |
| 2 | 2Hex1Pent | 474.17 | 9.187 | 0.88 | 6.162 |
| 3 | 2Hex1Pent | 474.17 | 10.653 | 0.54 | 7.145 |
| 4 | 2Hex1Pent | 474.17 | 12.713 | 0.37 | 8.526 |
| 5 | 3Hex | 504.18 | 1.491 | 0.84 | 1 |
| 6 | 3Hex | 504.18 | 2.193 | 0.35 | 1.471 |
| 7 | 3Hex | 504.19 | 6.695 | 4.31 | 4.49 |
| 8 | 3Hex | 504.19 | 7.637 | 2.06 | 5.122 |
| 9 | 3Hex | 504.18 | 8.885 | 1.94 | 5.959 |
| 10 | 3Hex | 504.19 | 9.605 | 1.74 | 6.442 |
| 11 | 3Hex | 504.19 | 12.868 | 1.45 | 8.63 |
| 12 | 3Hex | 504.18 | 14.159 | 0.4 | 9.496 |
| 13 | 3Hex1Pent | 636.23 | 6.413 | 0.92 | 4.301 |
| 14 | 3Hex1Pent | 636.23 | 12.074 | 1.13 | 8.098 |
| 15 | 3Hex1Pent | 636.23 | 13.068 | 0.77 | 8.765 |
| 16 | 3Hex1Pent | 636.23 | 18.947 | 1.52 | 12.708 |
| 17 | 3Hex1Pent | 636.23 | 19.573 | 0.75 | 13.127 |
| 18 | 3Hex1Pent | 636.23 | 20.258 | 0.81 | 13.587 |
| 19 | 4Hex | 666.24 | 4.124 | 2.33 | 2.766 |
| 20 | 4Hex | 666.24 | 6.206 | 2.04 | 4.162 |
| 21 | 4Hex | 666.24 | 9.373 | 2.99 | 6.286 |
| 22 | 4Hex | 666.24 | 10.25 | 2.66 | 6.875 |
| 23 | 4Hex | 666.24 | 10.97 | 3.94 | 7.357 |
| 24 | 4Hex | 666.24 | 14.977 | 1.75 | 10.045 |
| 25 | 4Hex | 666.24 | 16.802 | 4.87 | 11.269 |
| 26 | 4Hex | 666.24 | 17.38 | 5.13 | 11.657 |
| 27 | 4Hex | 666.24 | 19.079 | 1.69 | 12.796 |
| 28 | 4Hex | 666.24 | 19.911 | 0.58 | 13.354 |
| 29 | 4Hex | 666.24 | 20.691 | 0.36 | 13.877 |
| 30 | 4Hex | 666.24 | 24.147 | 1.15 | 16.195 |
| 31 | 4Hex1Pent | 798.28 | 9.604 | 0.45 | 6.441 |
| 32 | 4Hex1Pent | 798.28 | 15.28 | 0.48 | 10.248 |
| 33 | 4Hex1Pent | 798.28 | 17.219 | 0.45 | 11.549 |
| 34 | 4Hex1Pent | 798.28 | 18.793 | 0.64 | 12.604 |
| 35 | 4Hex1Pent | 798.28 | 20.328 | 1.41 | 13.634 |
| 36 | 4Hex1Pent | 798.28 | 21.012 | 0.36 | 14.093 |
| 37 | 4Hex1Pent | 798.28 | 21.923 | 0.44 | 14.704 |
| 38 | 4Hex1Pent | 798.28 | 22.714 | 0.55 | 15.234 |
| 39 | 4Hex1Pent | 798.28 | 26.723 | 0.35 | 17.923 |
| 40 | 4Hex1Pent | 798.28 | 27.138 | 0.22 | 18.201 |
| 41 | 4Hex1Pent | 798.28 | 27.347 | 0.26 | 18.341 |
| 42 | 4Hex1Pent | 798.28 | 27.565 | 0.32 | 18.488 |
| 43 | 5Hex | 828.29 | 7.648 | 1.05 | 5.129 |
| 44 | 5Hex | 828.29 | 9.213 | 1.28 | 6.179 |
| 45 | 5Hex | 828.29 | 12.21 | 0.37 | 8.189 |
| 46 | 5Hex | 828.29 | 14.335 | 1.28 | 9.614 |
| 47 | 5Hex | 828.29 | 14.932 | 0.75 | 10.015 |
| 48 | 5Hex | 828.29 | 16.24 | 2.01 | 10.892 |
| 49 | 5Hex | 828.29 | 17.527 | 1.3 | 11.755 |
| 50 | 5Hex | 828.29 | 18.549 | 5.98 | 12.441 |
| 51 | 5Hex | 828.29 | 18.882 | 0.36 | 12.664 |
| 52 | 5Hex | 828.29 | 19.204 | 1.29 | 12.88 |
| 53 | 5Hex | 828.29 | 19.522 | 0.69 | 13.093 |
| 54 | 5Hex | 828.29 | 20.062 | 1.8 | 13.455 |
| 55 | 5Hex | 828.29 | 20.313 | 1.44 | 13.624 |
| 56 | 5Hex | 828.29 | 21.885 | 1.43 | 14.678 |
| 57 | 5Hex | 828.29 | 25.896 | 3.46 | 17.368 |
| 58 | 5Hex | 828.29 | 27.822 | 0.56 | 18.66 |
| 59 | 5Hex | 828.29 | 28.03 | 0.64 | 18.799 |
| 60 | 6Hex | 990.34 | 11.067 | 0.31 | 7.423 |
| 61 | 6Hex | 990.34 | 11.999 | 0.4 | 8.048 |
| 62 | 6Hex | 990.34 | 15.579 | 0.48 | 10.449 |
| 63 | 6Hex | 990.34 | 17.343 | 0.5 | 11.632 |
| 64 | 6Hex | 990.34 | 18.939 | 0.55 | 12.702 |
| 65 | 6Hex | 990.34 | 19.881 | 1.01 | 13.334 |
| 66 | 6Hex | 990.34 | 21.075 | 0.83 | 14.135 |
| 67 | 6Hex | 990.34 | 21.86 | 0.73 | 14.661 |
| 68 | 6Hex | 990.34 | 22.178 | 0.72 | 14.875 |
| 69 | 6Hex | 990.34 | 22.854 | 2.01 | 15.328 |
| 70 | 6Hex | 990.34 | 23.366 | 1.15 | 15.671 |
| 71 | 6Hex | 990.34 | 24.012 | 1.16 | 16.105 |
| 72 | 6Hex | 990.34 | 25.898 | 0.92 | 17.37 |
| 73 | 6Hex | 990.34 | 26.344 | 0.71 | 17.669 |
| 74 | 6Hex | 990.34 | 28.735 | 0.53 | 19.272 |
| 75 | 6Hex | 990.34 | 28.984 | 0.59 | 19.439 |
| 76 | 7Hex | 1152.39 | 17.924 | 0.18 | 12.021 |
| 77 | 7Hex | 1152.4 | 22.096 | 0.58 | 14.82 |
| 78 | 7Hex | 1152.39 | 22.626 | 0.44 | 15.175 |
| 79 | 7Hex | 1152.4 | 22.915 | 0.27 | 15.369 |
| 80 | 7Hex | 1152.39 | 23.686 | 0.42 | 15.886 |
| 81 | 7Hex | 1152.39 | 24.151 | 0.37 | 16.198 |
| 82 | 7Hex | 1152.39 | 24.537 | 0.74 | 16.457 |
| 83 | 7Hex | 1152.39 | 25.769 | 0.59 | 17.283 |
| 84 | 7Hex | 1152.39 | 27.136 | 0.51 | 18.2 |
| 85 | 7Hex | 1152.39 | 28.252 | 0.34 | 18.948 |
| 86 | 7Hex | 1152.4 | 28.633 | 0.57 | 19.204 |
| 87 | 8Hex | 1314.45 | 24.955 | 0.37 | 16.737 |

Locust bean gum refers to a polysaccharide with a 73% β-1,4 mannose backbone, with 23% decorated with β-1,4 galactose. The produced oligosaccharides possessed various structural features similar to those in the parent polysaccharide. The mannose composition being 72.91%, followed by galactose at 22.98% (Table 3A). With the glycosidic linkage composition being 4 mannose, 4,6 mannose and terminal mannose at 62.02%, 8.95% and 6.82% respectively, and terminal-galactose being 19.58% (Table 4A). 39 oligosaccharides were observed in the pool that ranged from 3 hexose to 7 hexoses in length. The most abundant structures represent 31Hex, 11.02 min; 4Hex 4.188 min; 4Hex1Pent, 9.688 min; 5Hex 7.755 min; 6Hex, 11.153 min; 7Hex, 13.293 min. The full list of oligosaccharide peaks and abundances are found in Table 18. The oligosaccharide pool can be further distinguished by its 1H-13C 2D-NMR (HSQC) fingerprint (FIG. 13). Prominent peaks include those described in Table 5A.

TABLE 18
Oligosaccharides generated from the COG depolymerization of Locust
bean gum. Hex refers to hexose sugars, Pent refers to pentose
sugars, HexA refers to hexuronic acid sugars, and Deoxyhex refers
to deoxyhexose sugars. Hexoses refer to galactose and mannose.

| | | | RT | Oligo | Retention |
|---|---|---|---|---|---|
| Compound | Identity | Mass | (min) | Wt % | Factor |
| 1 | 3Hex | 504.19 | 1.492 | 2.27 | 1 |
| 2 | 3Hex | 504.19 | 1.855 | 0.85 | 1.243 |
| 3 | 3Hex | 504.18 | 3.002 | 0.75 | 2.012 |
| 4 | 3Hex | 504.18 | 4.461 | 1.28 | 2.99 |
| 5 | 3Hex (nr) | 504.17 | 11.02 | 9.48 | 7.386 |
| 6 | 3Hex1Pent | 636.23 | 6.511 | 4.42 | 4.364 |
| 7 | 3Hex1Pent | 636.23 | 7.066 | 2.09 | 4.736 |
| 8 | 3Hex1Pent | 636.23 | 8.646 | 1.15 | 5.795 |
| 9 | 4Hex | 666.24 | 4.188 | 7.94 | 2.807 |
| 10 | 4Hex | 666.24 | 4.911 | 2.47 | 3.292 |
| 11 | 4Hex | 666.24 | 5.225 | 3.70 | 3.502 |
| 12 | 4Hex | 666.24 | 5.608 | 3.28 | 3.759 |
| 13 | 4Hex | 666.24 | 7.33 | 1.86 | 4.913 |
| 14 | 4Hex | 666.24 | 15.185 | 1.00 | 10.178 |
| 15 | 4Hex1Deoxyhex | 812.26 | 10.546 | 0.99 | 7.068 |
| 16 | 4Hex1HexA | 842.27 | 10.31 | 2.49 | 6.91 |
| 17 | 4Hex1Pent | 798.28 | 9.688 | 4.29 | 6.493 |
| 18 | 4Hex1Pent | 798.28 | 11.09 | 2.19 | 7.433 |
| 19 | 4Hex1Pent | 798.28 | 11.592 | 1.11 | 7.769 |
| 20 | 4Hex1Pent | 798.28 | 14.207 | 0.98 | 9.522 |
| 21 | 5Hex | 828.29 | 7.755 | 6.79 | 5.198 |
| 22 | 5Hex | 828.29 | 7.992 | 2.61 | 5.357 |
| 23 | 5Hex | 828.29 | 8.599 | 2.52 | 5.763 |
| 24 | 5Hex | 828.29 | 9.09 | 2.45 | 6.092 |
| 25 | 5Hex | 828.29 | 9.802 | 1.88 | 6.57 |
| 26 | 5Hex | 828.29 | 10.062 | 3.12 | 6.744 |
| 27 | 5Hex | 828.29 | 13.089 | 2.17 | 8.773 |
| 28 | 6Hex | 990.34 | 11.153 | 5.21 | 7.475 |
| 29 | 6Hex | 990.34 | 11.417 | 1.65 | 7.652 |
| 30 | 6Hex | 990.34 | 11.65 | 1.50 | 7.808 |
| 31 | 6Hex | 990.34 | 12.024 | 1.55 | 8.059 |
| 32 | 6Hex | 990.34 | 12.344 | 1.34 | 8.273 |
| 33 | 6Hex | 990.34 | 13.877 | 2.18 | 9.301 |
| 34 | 6Hex | 990.34 | 14.341 | 2.19 | 9.612 |
| 35 | 6Hex | 990.34 | 15.221 | 1.50 | 10.202 |
| 36 | 7Hex | 1152.39 | 13.293 | 3.21 | 8.91 |
| 37 | 7Hex | 1152.39 | 14.078 | 1.24 | 9.436 |
| 38 | 7Hex | 1152.39 | 15.273 | 1.03 | 10.237 |
| 39 | 7Hex | 1152.39 | 16.279 | 1.25 | 10.911 |

Corn fiber refers to a polysaccharide or mixture of polysaccharides derived from spent distillers' grain or other corn streams. In some aspects, corn fiber refers to the base-soluble material extracted from distillers' grain or other corn steams. In some aspects, corn fiber refers to the acid soluble material extracted from distillers' grain or other corn streams. In some aspects, corn fiber refers to the insoluble material from distillers' grain or other corn streams. The corn fiber oligosaccharides were comprised of 3.07% glucose, 6.78% galactose, 35.76% arabinose, and 48.68% xylose. (Table 3A). The glycosidic linkage composition comprised 5.83% 4-glucose, 16.33% 4-xylose, 6.21% 3,4-xylose, 25.05% terminal xylose, 27.79% terminal arabinose (Table 4A). 29 oligosaccharides were observed in the pool that ranged from 3 hexose to 12 hexoses in length. The most abundant structures represent 3Hex, 4.11 min; 4Hex 9.29 min; 5Hex 12.31 min; 6Hex, 14.058; 7Hex, 15.254 min; 8Hex, 16.394; 9Hex, 18.013 min; 10Hex, 21.99 min; 11Hex, 22.911; 12Hex, 24.55 min. The full list of oligosaccharide peaks and abundances are found in Table 19. The oligosaccharide pool can be further distinguished by its 1H-13C 2D-NMR (HSQC) fingerprint (FIG. 13), which showed similar cross section coordinates to arabinoxylan. Prominent peaks include those described in Table 5A.

TABLE 19
Oligosaccharides generated from the COG depolymerization of
Corn Fiber. Hex refers to hexose sugars, Pent refers to pentose
sugars, HexA refers to hexuronic acid sugars, and Deoxyhex
refers to deoxyhexose sugars. Pentoses refer to xylose and
arabinose. Hexose refers to glucose and galactose.

| | | | RT | Oligo | Retention |
|---|---|---|---|---|---|
| Compound | Identity | Mass | (min) | Wt % | Factor |
| 1 | 1Hex2Pent | 444.16 | 5.025 | 10.3 | 3.398 |
| 2 | 2Pent | 282.11 | 1.479 | 4.73 | 1 |
| 3 | 2Pent | 282.11 | 2.064 | 3.14 | 1.396 |
| 4 | 2Pent | 282.11 | 2.448 | 3.78 | 1.655 |
| 5 | 2Pent | 282.11 | 2.957 | 4.32 | 1.999 |
| 6 | 2Pent | 282.11 | 3.206 | 3.8 | 2.168 |
| 7 | 2Pent | 282.11 | 6.244 | 3.24 | 4.222 |
| 8 | 3Pent | 414.15 | 7.353 | 11.34 | 4.972 |
| 9 | 3Pent | 414.15 | 8.303 | 13.75 | 5.614 |
| 10 | 3Pent | 414.15 | 8.594 | 4.01 | 5.811 |
| 11 | 3Pent | 414.15 | 11.783 | 7.23 | 7.967 |
| 12 | 3Pent | 414.15 | 12.195 | 9.68 | 8.245 |
| 13 | 3Pent | 414.15 | 13.961 | 4.86 | 9.439 |
| 14 | 3Pent | 414.15 | 16.586 | 3.28 | 11.214 |
| 15 | 4Pent | 546.2 | 7.243 | 3.43 | 4.897 |
| 16 | 4Pent | 546.2 | 9.733 | 2.24 | 6.581 |
| 17 | 4Pent | 546.2 | 15.637 | 6.88 | 10.573 |

B-Glucan refers to a polysaccharide with a β-1,4 β-1,3 in a 4 to 1 ratio glucose backbone. This sample was derived from oat. The produced oligosaccharides possessed various structural features similar to those in the parent polysaccharide. The glucose composition was 97.04% (Table 3A). With the glycosidic linkage composition being 4-glucose, 3 glucose, and terminal glucose at 48.91%, 30.95%, and 17.06% respectively (Table 4A). 76 oligosaccharides were observed in the pool that ranged from a hexose a pentose to 6 hexoses in length. The most abundant structures represent 3Hex, 11.335 min, 15.193 min and 17.831 min; 4Hex, 21.557 min, 25.507 min and 26.403 min; Hex 29.82 min and 30.469 min. The full list of oligosaccharide peaks and abundances are found in Table 20. The oligosaccharide pool can be further distinguished by its 1H-13C 2D-NMR (HSQC). Prominent peaks include those described in Table 5A.

TABLE 20
Oligosaccharides generated from the COG depolymerization of
Oat β-glucan. Hex refers to hexose sugars, Pent refers
to pentose sugars, HexA refers to hexuronic acid sugars, and
Deoxyhex refers to deoxyhexose sugars. Hexose sugars refer
solely to glucose. Pentose sugars refer solely to arabinose.

| | | | RT | Oligo | Retention |
|---|---|---|---|---|---|
| Compound | Identity | Mass | (min) | Wt % | Factor |
| 1 | 1Hex1Pent | 314.1214 | 1.839 | 0.09 | 1.196 |
| 2 | 1Hex2Pent | 446.1638 | 14.618 | 0.46 | 9.511 |
| 3 | 1Hex2Pent | 446.1641 | 14.73 | 0.17 | 9.584 |
| 4 | 1Hex2Pent | 446.1635 | 15.648 | 0.28 | 10.181 |
| 5 | 1Hex2Pent | 446.1643 | 17.222 | 0.11 | 11.205 |
| 6 | 2Hex | 344.1321 | 1.537 | 1.05 | 1 |
| 7 | 2Hex | 344.1329 | 2.878 | 2.41 | 1.872 |
| 8 | 2Hex1Pent | 476.1744 | 12.931 | 1.06 | 8.413 |
| 9 | 2Hex1Pent | 476.174 | 13.229 | 0.49 | 8.607 |
| 10 | 2Hex1Pent | 476.1753 | 13.732 | 1.53 | 8.934 |
| 11 | 2Hex1Pent | 476.1742 | 15.112 | 0.33 | 9.832 |
| 12 | 2Hex1Pent | 476.1748 | 16.113 | 0.84 | 10.483 |
| 13 | 2Hex1Pent | 476.1742 | 16.527 | 0.33 | 10.753 |
| 14 | 2Hex2Pent | 608.2157 | 24.944 | 0.45 | 16.229 |
| 15 | 2Hex2Pent | 608.2161 | 26.779 | 0.19 | 17.423 |
| 16 | 3Hex | 506.1856 | 3.295 | 0.63 | 2.144 |
| 17 | 3Hex | 506.1856 | 4.549 | 0.8 | 2.96 |
| 18 | 3Hex | 506.1843 | 10.5 | 0.08 | 6.831 |
| 19 | 3Hex | 506.1855 | 11.335 | 7.55 | 7.375 |
| 20 | 3Hex | 506.1855 | 14.01 | 0.23 | 9.115 |
| 21 | 3Hex | 506.1857 | 15.193 | 9.74 | 9.885 |
| 22 | 3Hex | 506.1857 | 15.445 | 0.05 | 10.049 |
| 23 | 3Hex | 506.184 | 15.874 | 0.08 | 10.328 |
| 24 | 3Hex | 506.1856 | 17.831 | 6.41 | 11.601 |
| 25 | 3Hex1HexA | 682.2171 | 25.124 | 0.13 | 16.346 |
| 26 | 3Hex1HexA | 682.2176 | 27.182 | 0.58 | 17.685 |
| 27 | 3Hex1HexA | 682.2177 | 27.272 | 0.19 | 17.744 |
| 28 | 3Hex1HexA | 682.2169 | 28.058 | 0.67 | 18.255 |
| 29 | 3Hex1Pent | 638.2261 | 9.335 | 0.1 | 6.074 |
| 30 | 3Hex1Pent | 638.2263 | 10.131 | 0.07 | 6.591 |
| 31 | 3Hex1Pent | 638.2276 | 23.161 | 0.93 | 15.069 |
| 32 | 3Hex1Pent | 638.2275 | 23.637 | 0.4 | 15.379 |
| 33 | 3Hex1Pent | 638.2273 | 24.298 | 0.44 | 15.809 |
| 34 | 3Hex1Pent | 638.2271 | 25.161 | 0.72 | 16.37 |
| 35 | 3Hex1Pent | 638.2282 | 25.768 | 0.84 | 16.765 |
| 36 | 3Hex1Pent | 638.2271 | 26.311 | 0.23 | 17.118 |
| 37 | 3Hex2Pent | 770.2696 | 29.593 | 0.06 | 19.254 |
| 38 | 3Hex2Pent | 770.2686 | 30.152 | 0.19 | 19.617 |
| 39 | 3Hex2Pent | 770.2681 | 30.838 | 0.07 | 20.064 |
| 40 | 3Hex2Pent | 770.2695 | 30.903 | 0.12 | 20.106 |
| 41 | 4Hex | 668.237 | 7.62 | 0.19 | 4.958 |
| 42 | 4Hex | 668.238 | 8.72 | 0.1 | 5.673 |
| 43 | 4Hex | 668.237 | 9.276 | 1.35 | 6.035 |
| 44 | 4Hex | 668.2351 | 9.819 | 0.06 | 6.388 |
| 45 | 4Hex | 668.2378 | 10.269 | 1.06 | 6.681 |
| 46 | 4Hex | 668.2379 | 10.63 | 0.34 | 6.916 |
| 47 | 4Hex | 668.2388 | 21.557 | 8.22 | 14.025 |
| 48 | 4Hex | 668.2386 | 24.211 | 0.1 | 15.752 |
| 49 | 4Hex | 668.239 | 25.244 | 4.57 | 16.424 |
| 50 | 4Hex | 668.239 | 25.507 | 5.92 | 16.595 |
| 51 | 4Hex | 668.2387 | 26.403 | 6.18 | 17.178 |
| 52 | 4Hex | 668.2343 | 26.607 | 0.04 | 17.311 |
| 53 | 4Hex1Deoxyhex | 814.2585 | 31.081 | 0.31 | 20.222 |
| 54 | 4Hex1HexA | 844.247 | 29.807 | 0.04 | 19.393 |
| 55 | 4Hex1Pent | 800.2787 | 28.827 | 0.23 | 18.755 |
| 56 | 4Hex1Pent | 800.2797 | 29.062 | 0.41 | 18.908 |
| 57 | 4Hex1Pent | 800.2787 | 29.552 | 0.11 | 19.227 |
| 58 | 4Hex1Pent | 800.2799 | 29.615 | 0.56 | 19.268 |
| 59 | 4Hex1Pent | 800.2788 | 30.227 | 1.2 | 19.666 |
| 60 | 5Hex | 830.2911 | 11.751 | 0.2 | 7.645 |
| 61 | 5Hex | 830.2895 | 13.261 | 0.48 | 8.628 |
| 62 | 5Hex | 830.2893 | 13.518 | 0.46 | 8.795 |
| 63 | 5Hex | 830.2899 | 13.84 | 0.71 | 9.005 |
| 64 | 5Hex | 830.2907 | 27.932 | 2.29 | 18.173 |
| 65 | 5Hex | 830.2922 | 28.423 | 3.57 | 18.493 |
| 66 | 5Hex | 830.2907 | 29.322 | 1.02 | 19.077 |
| 67 | 5Hex | 830.2908 | 29.82 | 4.98 | 19.401 |
| 68 | 5Hex | 830.291 | 30.469 | 6.13 | 19.824 |
| 69 | 6Hex | 992.3433 | 14.683 | 0.1 | 9.553 |
| 70 | 6Hex | 992.342 | 15.905 | 0.98 | 10.348 |
| 71 | 6Hex | 992.3433 | 31.896 | 1.52 | 20.752 |
| 72 | 6Hex | 992.3427 | 31.937 | 1.02 | 20.779 |
| 73 | 6Hex | 992.3434 | 33.337 | 0.52 | 21.69 |
| 74 | 6Hex | 992.3429 | 33.37 | 1.9 | 21.711 |
| 75 | 6Hex | 992.3429 | 33.763 | 0.91 | 21.967 |
| 76 | 6Hex | 992.3421 | 35.487 | 1.12 | 23.088 |

### Example 10B Pipeline 2

### (Composition of Matter)

A number of polysaccharide rich materials were assessed for their ability to be dissociated by COG. Each material produced a number of unexpected oligosaccharide products, due to the prior lack of mechanism, and were characterized at both the pool level (multiple oligosaccharides) and the individual oligosaccharide level. When possible, the pools are described by their monosaccharide and glycosidic linkage profiles, 2D-NMR (Table 5B), and liquid chromatography/quadrapole-time-of-flight mass spectrometry (LC/Q-TOF MS). Furthermore, individual oligosaccharides were identified and characterized by their mass, retention time, and fragmentation patterns.

Oligosaccharide production: Karaya Gum, Yeast mannan extract, Orange Fiber, Tragacanth Gum, Tomato peels, Pea Fiber, Xanthan Gum, Spent Coffee Grounds, Gellan Gum, Soy Fiber, Carrot fiber, sugar cane, pacific kelp powder, Sea Lettuce Powder, Olive liquid waste, Beet pectin, and Baobab powder (550 mg) were dissolved in 20 ml of HPLC grade water in a capped reaction vessel and placed in a shaker-incubator for 20 min at 55° C. and 85 RPM. The pH of the solution was adjusted to 5.2. Hydrogen peroxide (5 ml) and iron (II) sulfate (2.75 mg in 50 μL water) were added to the reaction mixture and mixed thoroughly. The reaction in the capped reaction vessel proceeded in the shaker-incubator at 55° C. and 65 RPM for two hours. The capped reaction cooled to 12° C. in a −20° C. freezer. Ammonium hydroxide (1 ml of 28% v/v to pH 10.2) was used to adjust pH and sample was reacted at 45° C. in a shaker-incubator for 1 hour at 20 RPM, the cap was left loose to allow oxygen, ammonia, and carbon dioxide gases to be released. The sample is then frozen and lyophilized, then stored at −80° C. The freeze-dried oligosaccharide mixture was rehydrated with the minimum amount of water required to allow for a free-flowing solution. This solution was then loaded onto a column containing 15 mL mixed bed ion exchange resin per gram (dry weight) of crude material, and the runoff was collected in a plastic freezer bag. Once the material was loaded onto the column, the column was then rinsed with 3 bed volumes of water. Finally, the runoff was sealed and frozen in the bag, then carefully shattered and subjected to lyophilization.

Monosaccharide analysis was performed in the manner of Amicucci et al. (Amnicucci, M. J., Galermo, A. G., et al. (2019). International Journal of Mass Spectrometry 438: 22-28.) but was adapted to be run on an Agilent 6530 Q-TOF mass spectrometer. Glycosidic linkage analysis was performed in the manner of Galerro, A. G., Nandita, E., et al. (2018). Analytical Chemistry 90(21): 13073-13080 with the expanded retention time library presented in Galermo, A. G., Nandita, E., et al. (2019). Analytical Chemist 91(20): 13022-13031 and was adapted to be run on an Agilent 6530 Q-TOF mass spectrometer. Oligosaccharide analysis was performed in the manner of Amnicucci, M. J., Nandita, E., et al. (2020). Nature Communications 11(1): 1-12. Oligosaccharide peak volumes were generated from Agilent Mass Hunter Qualitative Analysis B.10 by using their “find by molecular feature” function. For NMR analysis, oligosaccharides were dissolved in D₂O at a concentration of 50 mg/ml and were analyzed on a 600 MHz Bruker NMR spectrometer for their HSQC spectra.

Monosaccharide Composition: The oligosaccharide pools generated from the COG reaction were analyzed for their monosaccharide compositions, which are shown in Table 3B. Seven monosaccharides were measured in the 17 samples that underwent the COG reactions.

TABLE 3B
Monosaccharide composition of claimed composition of matter pools. Units represent
relative abundance by mass. The notation “—” represents a monosaccharide
that exists in an amount less than 2% (which can be 0%) of the total polysaccharide weight.

| | Glucose | Galactose | Xylose | Arabinose | Fucose | Rhamnose | GlcA | GalA | Mannose | Others |
|---|---|---|---|---|---|---|---|---|---|---|
| Olive | 11.55 | 13.48 | 3.97 | 55.25 | — | 6.19 | — | 5.46 | 3.08 | 0.65 |
| liquid waste | | | | | | | | | | |
| Tragacanth | 5.76 | 19.28 | 5.59 | 60.14 | 2.20 | 2.96 | — | 3.76 | — | 0.24 |
| Gum | | | | | | | | | | |
| Sea Lettuce | 13.46 | 3.62 | 13.95 | — | — | 62.96 | 4.10 | — | — | 0.45 |
| Powder | | | | | | | | | | |
| Pacific | 13.04 | 9.55 | 16.21 | — | 55.35 | — | — | — | 3.44 | 0.3 |
| Kelp powder | | | | | | | | | | |
| Tomato peels | 13.34 | 21.80 | 2.99 | 32.93 | — | 11.24 | — | 15.19 | — | 0.63 |
| Soy Fiber | 2.78 | 49.61 | 4.82 | 34.07 | 2.23 | 2.42 | — | 2.94 | — | 0.67 |
| Xanthan Gum | 63.86 | — | — | — | — | — | 2.50 | — | 30.64 | 0.37 |
| Pea Fiber | 4.01 | 9.53 | — | 79.76 | — | 2.48 | — | 3.54 | — | 0.24 |
| Sugar cane | 35.32 | 2.30 | 47.48 | 12.24 | — | — | — | — | — | 1.33 |
| fiber | | | | | | | | | | |
| Yeast mannan | 28.53 | — | — | — | — | — | — | — | 70.69 | 0.48 |
| extract | | | | | | | | | | |
| Gellan Gum | 44.29 | — | — | — | — | 42.94 | 7.70 | — | — | 1.37 |
| Spent Coffee | 6.64 | 49.81 | — | 14.03 | — | — | — | — | 25.99 | 1.38 |
| Grounds | | | | | | | | | | |
| Carrot fiber | 13.58 | 25.82 | — | 56.85 | — | — | — | — | — | 1.21 |
| (SP) | | | | | | | | | | |
| Orange Fiber | 11.45 | 9.82 | — | 51.27 | — | 9.60 | 6.82 | 9.30 | — | 0.48 |
| Karaya Gum | — | 42.48 | — | — | — | 37.02 | — | 16.15 | — | 0.50 |
| Yeast Beta- | 96.37 | — | — | — | — | — | — | — | — | 0.27 |
| glucan | | | | | | | | | | |
| Beet Pectin | 3.00 | 53.14 | — | 14.59 | — | 11.53 | — | 13.78 | — | 2.14 |
| Baobab powder | 35.03 | 12.4 | 17.94 | 8.34 | 2.81 | 4.33 | — | 12.1 | 5.97 | 0.95 |
| Lupin galactan | — | 77.18 | — | 8.99 | — | 3.37 | — | — | — | 6.94 |
| Desatrched | 9.78 | 5.47 | — | 76.85 | — | 2.89 | — | 3.26 | — | 0.24 |
| Pea Fiber | | | | | | | | | | |

Glycosidic Linkage Analysis: The oligosaccharide pools generated from the COG reaction were analyzed for their monosaccharide compositions, which are shown in Table 4B. Sixteen glycosidic linkage positions were identified in the 20 samples that underwent the COG reactions.

TABLE 4B
Glycosidic linkage compositions of claimed composition of matter pools. Units represent
relative abundance by peak area. The notation “—” represents a glycosidic linkage
that exists in an amount less than 2% (which can be 0%) of the total polysaccharide weight.

| | | | | 4,6- | | | | | | 3,6- | T- |
|---|---|---|---|---|---|---|---|---|---|---|---|
| | 3-Glc | 4-Glc | 6-Glc | Glc | T-Glc | T-Man | 3-Gal | 4-Gal | 6-Gal | Gal | Gal-p |
| Olive | — | — | — | — | 4.32 | — | — | 4.73 | — | — | 2.36 |
| liquid waste | | | | | | | | | | | |
| Tragacanth | — | 6.74 | — | — | 2.41 | — | — | 2.46 | — | 4.23 | — |
| Gum | | | | | | | | | | | |
| Sea Lettuce | 5.98 | 46.48 | — | — | 15.29 | — | — | — | — | — | 3.55 |
| Powder | | | | | | | | | | | |
| Pacific | 10.16 | — | 7.79 | — | 10.9 | 2.88 | — | — | — | — | 2.1 |
| Kelp powder | | | | | | | | | | | |
| Tomato peels | — | 2.91 | — | — | 5.24 | — | — | 14.55 | — | — | 3.94 |
| Soy Fiber | — | 3.28 | — | — | — | — | — | 41.54 | — | — | 5.28 |
| Xanthan Gum | 3.28 | 29.88 | — | — | 11.54 | 5.53 | — | — | — | 12.24 | — |
| Pea Fiber | — | 3.4 | — | — | — | — | — | 4.54 | — | — | 2.89 |
| Sugar cane | 3.44 | 15.54 | — | — | 8.94 | — | — | — | — | — | — |
| fiber | | | | | | | | | | | |
| Yeast mannan | 5.39 | 12 | 4.66 | — | 9.95 | 24.64 | — | — | — | — | — |
| extract | | | | | | | | | | | |
| Gellan Gum | 20.96 | 24.24 | — | — | 10.25 | — | — | — | — | — | — |
| Spent Coffee | — | — | — | — | 2.26 | 8.86 | 9.37 | — | 3.78 | 6.98 | 9.13 |
| Grounds | | | | | | | | | | | |
| Carrot fiber | — | 2.26 | — | — | 5.25 | — | — | 17.73 | — | — | 4.56 |
| (SP) | | | | | | | | | | | |
| Orange Fiber | — | — | — | — | 6.2 | — | — | 4.88 | — | — | 2.47 |
| Karaya Gum | — | — | — | — | — | — | — | 13.1 | — | — | 29.64 |
| Yeast Beta- | 35.52 | 21.18 | 14.72 | — | 18.75 | — | — | — | — | — | — |
| glucan | | | | | | | | | | | |
| Beet Pectin | — | 3.27 | — | — | — | — | — | 26.45 | 6 | 3 | 11.19 |
| Baobab powder | — | 9.03 | — | 4.18 | 2.02 | — | — | — | — | — | 2.85 |
| Lupin Galactan | — | — | — | — | — | — | — | 61 | — | — | 21.2 |
| Destarched | — | 3.71 | — | — | — | — | — | — | — | — | — |
| Pea Fiber | | | | | | | | | | | |

| | | 4,6- | | | | | | 2,3,4- | 3,4- | | 3,5- |
|---|---|---|---|---|---|---|---|---|---|---|---|
| | 2-Man | Man | 3-Man | 4-Man | 2-Xyl | T-Xyl | 4-Xyl | Xyl | Xyl | 5-Arab | Arab |
| Olive | — | — | — | — | — | — | — | — | — | 20.97 | 13.51 |
| liquid waste | | | | | | | | | | | |
| Tragacanth | — | — | — | — | 3.19 | 3.91 | — | — | — | 6.8 | 2.44 |
| Gum | | | | | | | | | | | |
| Sea Lettuce | 5.19 | — | — | — | — | — | 7.39 | — | — | — | — |
| Powder | | | | | | | | | | | |
| Pacific | — | — | — | — | 4.65 | 14.16 | 5.87 | — | — | — | — |
| Kelp powder | | | | | | | | | | | |
| Tomato peels | — | — | — | — | — | — | 2.65 | — | — | 29.15 | 2.91 |
| Soy Fiber | — | — | — | — | — | — | — | — | — | 11.11 | 4.81 |
| Xanthan Gum | 16.02 | 6.09 | — | — | — | — | — | — | — | — | — |
| Pea Fiber | — | — | — | — | — | — | — | — | — | 23.16 | 4.2 |
| Sugar cane | — | — | — | — | — | 5.97 | 27.17 | — | 4.81 | 7 | — |
| fiber | | | | | | | | | | | |
| Yeast mannan | 23.39 | — | 2.44 | 2.44 | — | — | — | — | — | — | — |
| extract | | | | | | | | | | | |
| Gellan Gum | — | — | — | — | — | — | — | — | — | — | — |
| Spent Coffee | — | — | — | 22.84 | — | — | — | — | — | 4.87 | — |
| Grounds | | | | | | | | | | | |
| Carrot fiber | — | — | — | — | — | — | — | — | — | 18.64 | 7.89 |
| (SP) | | | | | | | | | | | |
| Orange Fiber | — | — | — | — | — | — | — | — | — | 26.67 | 12.27 |
| Karaya Gum | — | — | — | — | — | — | — | — | — | — | — |
| Yeast Beta- | — | — | — | — | — | — | — | — | — | — | — |
| glucan | | | | | | | | | | | |
| Beet Pectin | — | — | — | — | — | — | — | — | — | 4.07 | — |
| Baobab powder | — | — | — | — | 10.55 | 15.49 | 5.82 | 13.06 | — | 2.71 | — |
| Lupin Galactan | — | — | — | — | — | — | — | — | — | 5.3 | — |
| Destarched | — | — | — | — | — | — | — | — | — | 21.21 | 5.13 |
| Pea Fiber | | | | | | | | | | | |

| | | | | | | | | | | 2,x,x- | |
|---|---|---|---|---|---|---|---|---|---|---|---|
| | | | | | | x-Fuc | | | | Rham | |
| | 2,5- | 2,3,5- | T- | | | (9.503 | | | | (1.626 | |
| | Arab | Arab | Arab-f | 2-Fuc | T-Fuc | min) | 2-Rham | 4-Rham | T-Rham | min) | 4-GlcA |
| Olive | — | — | 21.67 | — | — | — | — | — | — | — | — |
| liquid waste | | | | | | | | | | | |
| Tragacanth | 4.32 | 8.1 | 25.46 | — | 2.37 | — | — | — | — | — | — |
| Gum | | | | | | | | | | | |
| Sea Lettuce | — | — | — | — | — | — | — | — | — | — | — |
| Powder | | | | | | | | | | | |
| Pacific | — | — | — | 4.97 | 13.71 | 5.62 | — | — | — | — | — |
| Kelp powder | | | | | | | | | | | |
| Tomato peels | — | — | 7.57 | — | — | — | 8.69 | — | — | — | — |
| Soy Fiber | 1.31 | 2.2 | 13.98 | — | — | — | — | — | — | — | — |
| Xanthan Gum | — | — | — | — | — | — | — | — | — | — | 4.12 |
| Pea Fiber | 9.25 | 4.38 | 34.99 | — | — | — | — | — | — | — | — |
| Sugar cane | — | — | 8.31 | 5.97 | — | — | — | — | — | — | — |
| fiber | | | | | | | | | | | |
| Yeast mannan | — | — | — | — | — | — | — | — | — | — | — |
| extract | | | | | | | | | | | |
| Gellan Gum | — | — | — | — | — | — | — | 21.57 | 7.33 | — | 4.91 |
| Spent Coffee | — | — | 15.54 | — | — | — | — | — | — | — | — |
| Grounds | | | | | | | | | | | |
| Carrot fiber | 3.56 | 3.54 | 27.39 | — | — | — | — | — | — | — | — |
| (SP) | | | | | | | | | | | |
| Orange Fiber | — | — | 20.64 | — | — | — | 3.66 | — | — | — | — |
| Karaya Gum | — | — | 4.02 | — | — | — | 16.16 | — | 11.31 | — | — |
| Yeast Beta- | — | — | — | — | — | — | — | — | — | — | — |
| glucan | | | | | | | | | | | |
| Beet Pectin | — | — | 7 | — | — | — | 9.53 | — | — | — | — |
| Baobab powder | — | 4.67 | 4.25 | — | — | — | — | 2.21 | — | 2.17 | — |
| Lupin Galactan | — | — | 4.06 | — | — | — | — | — | — | — | — |
| Destarched | 6.36 | 3.27 | 47.11 | — | — | — | — | — | — | — | — |
| Pea Fiber | | | | | | | | | | | |

| | | | 2,x-Hex | x-Hex | 2-Pent | x,x-Hex | x-Pent | x-Pent | 2,x-Pent | |
|---|---|---|---|---|---|---|---|---|---|---|
| | | | (1.628 | (2.108 | (2.769 | (3.755 | (5.289 | (6.448 | (3.346 | |
| | T-GlcA | T-GalA | min) | min) | min) | min) | min) | min) | min) | Other |
| Olive | — | — | — | — | — | — | 7.88 | — | — | 1.55 |
| liquid waste | | | | | | | | | | |
| Tragacanth | — | — | — | 2.55 | 2.37 | — | 3.3 | 5.92 | — | 4.97 |
| Gum | | | | | | | | | | |
| Sea Lettuce | — | — | — | — | — | — | — | — | — | 3.54 |
| Powder | | | | | | | | | | |
| Pacific | — | — | — | — | — | 2.95 | — | — | — | 2.55 |
| Kelp powder | | | | | | | | | | |
| Tomato peels | — | — | — | 3.45 | — | — | — | — | — | 2.73 |
| Soy Fiber | — | — | — | — | — | — | — | — | — | 2.26 |
| Xanthan Gum | — | — | — | — | — | — | — | — | — | 1.51 |
| Pea Fiber | — | — | — | — | — | — | — | — | — | 0.45 |
| Sugar cane | 2 | — | — | — | — | — | — | — | — | 2.03 |
| fiber | | | | | | | | | | |
| Yeast mannan | — | — | 7.21 | — | — | 2.2 | — | — | — | 1.45 |
| extract | | | | | | | | | | |
| Gellan Gum | 2.5 | — | — | — | — | — | — | — | — | 1.16 |
| Spent Coffee | — | — | — | — | — | — | — | 2.04 | — | 2.37 |
| Grounds | | | | | | | | | | |
| Carrot fiber | — | — | — | — | — | — | — | — | — | 2.6 |
| (Small | | | | | | | | | | |
| Particle) | | | | | | | | | | |
| Orange Fiber | — | — | — | — | — | — | 2.89 | 2.34 | — | 1.76 |
| Karaya Gum | 11.18 | — | — | 5.94 | — | — | — | — | — | 3.26 |
| Yeast Beta- | — | — | — | — | — | 5.68 | — | — | — | 0.9 |
| glucan | | | | | | | | | | |
| Beet Pectin | 2.85 | 3.31 | — | 6.07 | 3.84 | — | — | — | — | 1.76 |
| Baobab powder | — | — | — | — | — | — | — | — | 2.64 | 5.39 |
| Lupin Galactan | — | — | — | — | — | — | — | — | — | 2.38 |
| Destarched | — | — | — | — | — | — | — | — | — | 4.65 |
| Pea Fiber | | | | | | | | | | |

1H-13C HSQC NMR: Was performed on all of the samples. The analysis provided a fingerprint of each sample in order to compare the similarities between these and future oligosaccharide pools. The cross peak coordinates found in the anomeric region of the spectra are listed in Table 5B and some of the spectra are provided in FIG. 13B.

TABLE 5B
1H-13C HSQC NMR correlations from oligosaccharides created from the COG process.
The listed pairs correspond to those major peaks in the anomeric region.

| | | Yeast mannan | | | | | | | |
|---|---|---|---|---|---|---|---|---|---|
| Karaya Gum | | extract | | Orange Fiber | | Tragacanth Gum | | Yeast β-Glucan | |
| ¹H δ | ¹³C δ | ¹H δ | ¹³C δ | ¹H δ | ¹³C δ | ¹H δ | ¹³C δ | ¹H δ | ¹³C δ |
| ppm | ppm | ppm | ppm | ppm | ppm | ppm | ppm | ppm | ppm |
| 5.12 | 101.1 | 5.02 | 101.63 | 5 | 107.84 | 5.18 | 107.78 | 5.02 | 100.98 |
| 5.09 | 101.1 | 5.00 | 101.84 | 4.94 | 108.46 | 5.06 | 109.56 | 5.00 | 101.20 |
| 5.09 | 98.63 | 4.88 | 94.17 | 4.91 | 108.23 | 5.00 | 107.20 | 4.97 | 92.22 |
| 4.95 | 91.23 | 4.87 | 103.04 | 4.88 | 99.79 | 4.93 | 107.66 | 4.90 | 92.69 |
| 4.87 | 93.56 | 4.85 | 95.35 | 4.86 | 108.35 | 4.91 | 106.92 | 4.88 | 93.58 |
| 4.80 | 94.48 | 4.85 | 103.17 | 4.84 | 107.88 | 4.91 | 107.51 | 4.52 | 103.56 |
| 4.21 | 97.95 | 4.83 | 103.35 | 4.83 | 99.88 | 4.86 | 107.60 | 4.48 | 103.67 |
| 4.21 | 75.84 | 4.52 | 104.17 | 4.82 | 109.47 | 4.83 | 107.17 | 4.43 | 103.91 |
| 4.20 | 75.93 | 4.36 | 104.92 | 4.77 | 109.16 | 4.82 | 108.62 | 4.37 | 96.82 |
| 4.18 | 106.63 | 4.24 | 104.33 | 4.55 | 101.69 | 4.77 | 108.40 | 4.36 | 96.82 |
| 4.17 | 76.11 | 3.98 | 69.44 | 4.53 | 101.58 | 4.19 | 103.96 | 4.36 | 104.37 |
| 4.00 | 70.95 | 3.57 | 69.49 | 4.26 | 97.96 | 4.17 | 103.93 | 4.34 | 103.06 |
| 3.96 | 70.9 | 3.71 | 67.11 | 4.26 | 106.53 | 3.66 | 67.03 | 4.33 | 104.58 |
| | | 3.66 | 67.08 | | | 3.64 | 67.40 | 4.30 | 104.54 |
| | | 3.53 | 66.55 | | | 3.55 | 67.04 | 4.29 | 104.56 |
| | | | | | | 3.52 | 67.38 | 4.27 | 97.38 |
| | | | | | | | | 4.26 | 97.38 |
| | | | | | | | | 4.24 | 103.78 |
| | | | | | | | | 4.18 | 103.65 |
| | | | | | | | | 4.17 | 103.59 |
| | | | | | | | | 4.15 | 104.04 |
| | | | | | | | | 3.98 | 68.80 |
| | | | | | | | | 3.57 | 68.80 |
| | | | | | | | | 3.46 | 86.75 |
| | | | | | | | | 3.42 | 87.28 |
| | | | | | | | | 3.35 | 88.43 |
| | | | | | | | | 3.44 | 79.92 |

| | | | | | | Spent Coffee | | | | Olive | |
|---|---|---|---|---|---|---|---|---|---|---|---|
| Tomato Peels | | Pea Fiber | | Xanthan Gum | | Grounds | | Gellan Gum | | liquid waste | |
| ¹H δ | ¹³C δ | ¹H δ | ¹³C δ | ¹H δ | ¹³C δ | ¹H δ | ¹³C δ | ¹H δ | ¹³C δ | ¹H δ | ¹³C δ |
| ppm | ppm | ppm | ppm | ppm | ppm | ppm | ppm | ppm | ppm | ppm | ppm |
| 5.18 | 92.81 | 5.00 | 107.17 | 5.01 | 93.22 | 5.06 | 109.5 | 5.12 | 99.92 | 4.96 | 107.92 |
| 5.09 | 101.64 | 4.93 | 107.83 | 5.00 | 101.92 | 4.90 | 94.30 | 5.08 | 100.49 | 4.91 | 108.24 |
| 5.06 | 99.35 | 4.90 | 105.88 | 4.97 | 102.02 | 4.77 | 108.67 | 5.05 | 100.46 | 4.83 | 102.59 |
| 5.04 | 93.46 | 4.90 | 106.90 | 4.97 | 102.29 | 4.76 | 108.4 | 5.01 | 100.49 | 4.82 | 102.53 |
| 4.90 | 108.24 | 4.86 | 107.67 | 4.91 | 93.19 | 4.53 | 101.16 | 5.00 | 101.01 | 4.82 | 109.43 |
| 4.89 | 99.78 | 4.83 | 107.22 | 4.88 | 94.14 | 4.49 | 104.7 | 4.97 | 100.95 | 4.8 | 109.32 |
| 4.85 | 99.78 | 4.81 | 108.63 | 4.87 | 95.07 | 4.47 | 101.16 | 4.90 | 92.50 | 4.78 | 109.27 |
| 4.82 | 99.74 | 4.77 | 107.44 | 4.74 | 101.59 | 4.40 | 105.35 | 4.88 | 92.79 | 4.76 | 109.21 |
| 4.82 | 102.31 | 4.77 | 108.55 | 4.73 | 99.99 | 4.20 | 103.98 | 4.80 | 94.54 | 7.75 | 109.11 |
| 4.82 | 109.49 | 4.26 | 105.83 | 4.71 | 101.77 | 4.15 | 104.22 | 4.43 | 104.69 | 4.57 | 99.93 |
| 4.77 | 109.21 | 4.04 | 85.79 | 4.66 | 99.74 | 3.88 | 103.50 | 4.36 | 105.04 | | |
| 4.74 | 109.17 | 3.92 | 87.99 | 4.64 | 99.82 | 3.74 | 112.41 | 4.30 | 102.1 | | |
| 4.73 | 98.21 | 4.63 | 101.54 | 3.42 | 99.83 | 4.29 | 97.07 | 3.93 | 88.01 | | |
| 4.67 | 100.78 | 4.38 | 103.77 | 3.22 | 97.84 | 4.29 | 104.52 | 3.75 | 79.18 | | |
| 4.63 | 100.85 | 4.34 | 103.32 | | | 4.24 | 102.68 | 3.70 | 67.26 | | |
| 4.29 | 98.28 | 4.31 | 97.82 | | | | | 3.65 | 66.74 | | |
| 4.26 | 98.14 | 4.31 | 103.77 | | | | | 3.63 | 67.51 | | |
| 4.27 | 102.81 | 4.27 | 98.04 | | | | | 3.54 | 67.16 | | |
| 4.26 | 106.53 | 4.25 | 103.47 | | | | | 3.54 | 66.61 | | |
| | | 3.84 | 64.91 | | | | | 3.52 | 67.48 | | |
| | | 3.66 | 64.86 | | | | | | | | |
| | | Carrot Fiber | | Sugar Cane | | Pacific Kelp | | Sea Lettuce | | | |
| Soy Fiber | | (SP) | | Fiber | | Powder | | Powder | | Beet Pectin | |
| ¹H δ | ¹³C δ | ¹H δ | ¹³C δ | ¹H δ | ¹³C δ | ¹H δ | ¹³C δ | ¹H δ | ¹³C δ | ¹H δ | ¹³C δ |
| ppm | ppm | ppm | ppm | ppm | ppm | ppm | ppm | ppm | ppm | ppm | ppm |
| 4.99 | 107.2 | 5.18 | 92.20 | 5.34 | 107.75 | 4.86 | 93.71 | 5.18 | 92.15 | 5.18 | 92.19 |
| 4.93 | 107.81 | 4.99 | 107.17 | 5.20 | 108.06 | 4.84 | 93.65 | 4.88 | 93.55 | 5.00 | 107.24 |
| 4.90 | 106.86 | 4.94 | 107.80 | 5.18 | 92.33 | 4.87 | 95.13 | 4.76 | 94.51 | 4.91 | 107.59 |
| 4.91 | 107.59 | 4.90 | 107.63 | 5.08 | 97.86 | 4.88 | 99.91 | 4.73 | 99.95 | 4.86 | 107.69 |
| 4.86 | 107.64 | 4.90 | 106.93 | 4.90 | 92.64 | 4.90 | 102.73 | 4.68 | 99.97 | 4.83 | 107.22 |
| 4.83 | 107.22 | 4.86 | 107.68 | 4.42 | 104.18 | 4.62 | 102.06 | 4.35 | 104.68 | 4.82 | 108.76 |
| 4.82 | 108.8 | 4.83 | 107.27 | 4.37 | 103.14 | 4.61 | 100.85 | 4.33 | 104.43 | 4.77 | 108.55 |
| 4.79 | 108.68 | 4.82 | 108.75 | 4.36 | 103.22 | 4.59 | 100.02 | 4.32 | 104.43 | 3.70 | 67.44 |
| 4.78 | 108.53 | 4.79 | 108.65 | 4.32 | 104.29 | 4.57 | 99.89 | 4.33 | 103.87 | 3.64 | 67.32 |
| 4.26 | 105.84 | 4.76 | 108.54 | 4.32 | 103.30 | 4.57 | 105.48 | 4.22 | 103.92 | 3.55 | 67.23 |
| 4.23 | 105.98 | 4.29 | 105.98 | 4.31 | 102.08 | 4.54 | 101.95 | 4.21 | 103.90 | 3.53 | 67.50 |
| 4.04 | 85.82 | 4.26 | 105.93 | 4.31 | 104.34 | 4.53 | 101.99 | | | | |
| 4.23 | 106.25 | 4.30 | 103.27 | 4.52 | 104.19 | | | | | | |
| 4.04 | 85.87 | 4.28 | 103.22 | 4.40 | 103.84 | | | | | | |
| 3.93 | 88.07 | 4.26 | 101.32 | 4.38 | 104.56 | | | | | | |
| 3.75 | 79.21 | 4.26 | 102.21 | 4.27 | 104.25 | | | | | | |
| 3.71 | 67.35 | 4.24 | 98.06 | 4.23 | 104.33 | | | | | | |
| 3.67 | 66.73 | 4.24 | 103.71 | 4.22 | 104.41 | | | | | | |
| 3.65 | 66.83 | 4.24 | 102.28 | 4.21 | 104.44 | | | | | | |
| 3.62 | 67.35 | 4.23 | 103.81 | 4.19 | 98.44 | | | | | | |
| 3.57 | 67.20 | 4.22 | 98.02 | 4.16 | 104.63 | | | | | | |
| 3.55 | 67.17 | 4.22 | 102.36 | 4.15 | 104.15 | | | | | | |
| 3.53 | 67.14 | 4.20 | 102.24 | 4.07 | 104.75 | | | | | | |
| 3.51 | 67.45 | 4.17 | 102.24 | | | | | | | | |
| | | 3.97 | 86.68 | | | | | | | | |
| | | 3.70 | 66.28 | | | | | | | | |
| | | 3.06 | 66.35 | | | | | | | | |

| | | |
|---|---|---|
| | Lupin | Destarched |
| Baobab Powder | Galactan | Pea Fiber |

| ¹H δ | ¹³C δ | ¹H δ | ¹³C δ | ¹H δ | ¹³C δ |
|---|---|---|---|---|---|
| ppm | ppm | ppm | ppm | ppm | ppm |
| 5.17 | 107.78 | 4.93 | 107.78 | 5.17 | 100.60 |
| 5.06 | 93.08 | 4.91 | 93.08 | 5.08 | 95.20 |
| 4.99 | 106.74 | 4.90 | 106.74 | 5.07 | 100.12 |
| 4.94 | 102.05 | 4.90 | 102.05 | 5.02 | 108.48 |
| 4.92 | 94.49 | 4.80 | 94.49 | 5.02 | 100.21 |
| 4.91 | 108.46 | 4.76 | 108.46 | 5.01 | 92.27 |
| 4.90 | 94.68 | 4.75 | 94.68 | 4.99 | 100.39 |
| 4.90 | 94.83 | 4.73 | 94.83 | 4.99 | 95.51 |
| 4.88 | 105.93 | 4.34 | 105.93 | 4.99 | 92.13 |
| 4.88 | 103.77 | 4.26 | 103.77 | 4.97 | 106.14 |
| 4.85 | 105.87 | 4.26 | 105.87 | 4.96 | 101.79 |
| 4.82 | 106.21 | 4.24 | 106.21 | 4.92 | 107.05 |
| 4.76 | 67.27 | 3.71 | 67.27 | 4.90 | 101.62 |
| 4.73 | 67.34 | 3.65 | 67.34 | 4.90 | 98.46 |
| 4.63 | 67.53 | 3.64 | 67.53 | 4.90 | 92.05 |
| 4.55 | 67.17 | 3.55 | 67.17 | 4.89 | 106.52 |
| 4.48 | 67.53 | 3.52 | 67.53 | 4.87 | 101.19 |
| 4.34 | 67.50 | 3.51 | 67.50 | 4.85 | 107.00 |
| 4.33 | 103.23 | | | 4.85 | 92.48 |
| 4.32 | 96.94 | | | 4.82 | 106.57 |
| 4.26 | 102.21 | | | 4.80 | 93.82 |
| 4.26 | 97.36 | | | 4.76 | 107.75 |
| 4.25 | 106.07 | | | 4.72 | 94.18 |
| 4.25 | 103.82 | | | 4.63 | 98.84 |
| 4.21 | 97.94 | | | 4.32 | 102.54 |
| 4.19 | 103.87 | | | 4.31 | 96.51 |
| | | | | 4.26 | 102.05 |
| | | | | 4.26 | 96.68 |
| | | | | 4.24 | 105.55 |
| | | | | 4.23 | 102.75 |
| | | | | 4.21 | 97.16 |

Oligosaccharide Analysis: Oligosaccharides are presented in two formats. Tables 21-37 show the “Find By Molecular Feature” data that shows the mass, retention time, composition, and oligosaccharide relative abundance. Additionally, we have provided annotated chromatograms for some of the oligosaccharides in FIG. 14B.

Karaya Gum refers to a polysaccharide with an α-1,4 and α-1,2 alternating backbone with β-1,4 and β-1,3 branches. Karaya gum can be extracted from trees in the family Stericulia. The produced oligosaccharides possessed various structural features similar to those in the parent polysaccharide. The galactose composition was 42.48%, 37.02% of the mass comprises rhamnose, and about 16% of the mass comprises Galacturonic acid (Table 3B) and a glycosidic linkage composition of 29.4% termainal, 13.1% 4-Galactose, 11.18% terminal and galacturonic acid (Table 4B). The oligosaccharide pool can be distinguished by it's 1H-13C 2D-NMR (HSQC) fingerprint (FIG. 13B). Prominent peaks include those described in Table 5B.

Yeast mannan extract refers to a polysaccharide with β-1,4 mannose backbone that is derived from the cell walls of yeast in the Saccharomyces Cerevisiae. The produced oligosaccharides possessed various structural features similar to those in the parent polysaccharide. The mannose composition was 70.69% and 28.53% glucose (Table 3B). With the glycosidic linkage composition being 24.64% terminal mannose, 23.39% 2-mannose, 12% 4-glucose, 9.95% terminal glucose, and 5.39% 3-glucose.(Table 4B). 12 oligosaccharides were observed in the pool that ranged from 3 pentose to 5 hexose in length. The most abundant structures represent 5Hex, 1.921 min; 4hex, 10.916 min and 24.788 min; 3Hex, 4.863 min and 13.16 min; The full list of oligosaccharide peaks and abundances are found in Table 21. The oligosaccharide pool can be further distinguished by it's 1H-13C 2D-NMR (HSQC) fingerprint (FIG. 13B). Prominent peaks include those described in Table 5B.

TABLE 21
Oligosaccharides generated from the COG depolymerization
of Yeast mannan extract. Hex refers to either Glucose or
Mannose, Pent refers to pentose sugars, HexA refers to
hexuronic acid sugars, and Deoxyhex refers to deoxyhexose
sugars. Hexose sugars are either glucose or mannose.

| | | | RT | Oligo | Retention |
|---|---|---|---|---|---|
| Compound | Identity | Mass | (min) | Wt % | Factor |
| 1 | 3Hex | 504.18 | 1.225 | 3.04 | 1 |
| 2 | 3Hex | 504.19 | 4.863 | 13.2 | 3.97 |
| 3 | 3Hex | 504.19 | 10.984 | 5.24 | 8.967 |
| 4 | 3Hex | 504.19 | 11.677 | 1.3 | 9.532 |
| 5 | 3Hex | 504.19 | 13.16 | 16.41 | 10.743 |
| 6 | 4Hex | 666.24 | 1.497 | 7.99 | 1.222 |
| 7 | 4Hex | 666.24 | 10.916 | 13.88 | 8.911 |
| 8 | 4Hex | 666.24 | 16.918 | 2.32 | 13.811 |
| 9 | 4Hex | 666.24 | 24.788 | 9.9 | 20.235 |
| 10 | 5Hex | 828.29 | 1.926 | 10.61 | 1.572 |
| 11 | 5Hex | 828.29 | 14.015 | 8.82 | 11.441 |
| 12 | 5Hex | 828.29 | 30.092 | 7.29 | 24.565 |

Orange Fiber refers to a polysaccharide with an 3,5 Arabinose backbone with 2 rhamnose and α-1,4 galacturonic acid in a 3 to 2 ratio of methylated galacturonic acid decorations. Orange fiber is derived from fiber in the peel and pomace of the orange fruit. The produced oligosaccharides possessed various structural features similar to those in the parent polysaccharide. The arabinose composition of 51.27%, 11.45% glucose, 9.82% galactose, 9.60 rhamnose and 9.30% galacturonic acid (Table 3B). With the glycosidic linkage composition being 5-arabinose, 3,5 arabinose, 2-rhamnose and terminal arabinose at 26.67%, 12.27%, 3.66% and 20.64% respectively (Table 4B). The oligosaccharide pool can be further distinguished by 1H-13C 2D-NMR (HSQC). Prominent peaks include those described in Table 5B.

Tragacanth gum refers to a polysaccharide with a α-1,4 galacturonic acid backbone with α-1,3 xylose which is capped 1 out of 2 branches with either α-1,2 fucose or α-1,2 galactose. Additionally, a second polysaccharide is present a α-1,6 backbone with α-1,3 arabinose branches, capped with α-1,5 arabinose. Tragacanth gum can be derived from sap from the genus Astragalus. The produced oligosaccharides possessed various structural features similar to those in the parent polysaccharide. The arabinose composition of 60.14% followed by 19.28 galactose, 5.76% glucose, 5.59 xylose and 3.76% Galacturonic acid (Table 3B). With the glycosidic linkage composition being terminal arabinose, 2,3,5 arabinose, 5 arabinose, 4-glucose, 3,6 galactose, and terminal xylose at 25.46%, 8.1%, 6.8%, 4.23% and 3.91% respectively (Table 4B). 33 oligosaccharides were observed in the pool that ranged from 1Hex2Pent to 7 hexoses in length. The most abundant structures represent 3Hex, 4.762 min; 3Pent, 6.251 min; 4Hex, 10.425 min; 4Pent 11.299 min; 1Hex3Pent, 11.892 min: 5Hex, 13.328 min. The full list of oligosaccharide peaks and abundances are found in Table 22. The oligosaccharide pool can be further distinguished by its 1H-13C 2D-NMR (HSQC) fingerprint (FIG. 13B). Prominent peaks include those described in Table 5B.

TABLE 22
Oligosaccharides generated from the COG depolymerization
of Tragacanth Gum. Hex refers to hexose sugars, Pent
refers to pentose sugars, HexA refers to hexuronic
acid sugars, and Deoxyhex refers to deoxyhexose sugars.
Hexose sugars are either galactose or glucose with
pentose sugars being arabinose or xylose.

| | | | RT | Oligo | Retention |
|---|---|---|---|---|---|
| Compound | Identity | Mass | (min) | Wt % | Factor |
| 1 | 1Hex2Pent | 444.16 | 4.516 | 2.42 | 1.432 |
| 2 | 1Hex2Pent | 444.16 | 4.734 | 1.56 | 1.501 |
| 3 | 1Hex2Pent | 444.16 | 4.739 | 2.63 | 1.503 |
| 4 | 1Hex3Pent | 576.2 | 11.892 | 6.24 | 3.77 |
| 5 | 1Hex3Pent | 576.25 | 14.374 | 2.45 | 4.557 |
| 6 | 2Hex1Pent | 474.17 | 3.674 | 1.36 | 1.165 |
| 7 | 2Hex1Pent | 474.17 | 4.732 | 1.99 | 1.5 |
| 8 | 2Hex1Pent | 474.17 | 14.418 | 1.73 | 4.571 |
| 9 | 2Hex2Pent | 606.22 | 9.863 | 0.96 | 3.127 |
| 10 | 2Hex2Pent | 606.21 | 14.391 | 1.19 | 4.563 |
| 11 | 3Hex | 504.18 | 3.154 | 1.11 | 1 |
| 12 | 3Hex | 504.18 | 4.762 | 7.88 | 1.51 |
| 13 | 3Hex (NR) | 504.22 | 14.19 | 0.98 | 4.499 |
| 14 | 3Hex1Pent | 636.22 | 8.485 | 0.97 | 2.69 |
| 15 | 3Hex1Pent | 636.23 | 10.474 | 2.34 | 3.321 |
| 16 | 3Pent | 414.15 | 3.893 | 4.61 | 1.234 |
| 17 | 3Pent | 414.15 | 5.253 | 1.61 | 1.666 |
| 18 | 3Pent | 414.15 | 6.251 | 7.19 | 1.982 |
| 19 | 3Pent | 414.15 | 8.361 | 2.53 | 2.651 |
| 20 | 3Pent | 414.15 | 10.568 | 3.66 | 3.351 |
| 21 | 4Hex | 666.24 | 7.796 | 1.29 | 2.472 |
| 22 | 4Hex | 666.24 | 10.425 | 10.58 | 3.305 |
| 23 | 4Hex1Pent | 798.28 | 11.282 | 0.99 | 3.577 |
| 24 | 4Hex1Pent | 798.28 | 13.425 | 1.83 | 4.256 |
| 25 | 4Pent | 546.19 | 11.299 | 7.59 | 3.582 |
| 26 | 4Pent | 546.19 | 15.593 | 1.2 | 4.944 |
| 27 | 4Pent | 546.19 | 17.068 | 2.27 | 5.412 |
| 28 | 5Hex | 828.29 | 10.82 | 0.71 | 3.431 |
| 29 | 5Hex | 828.29 | 13.328 | 7.84 | 4.226 |
| 30 | 5Hex1Pent | 960.33 | 15.093 | 1.21 | 4.785 |
| 31 | 6Hex | 990.34 | 15.002 | 5.27 | 4.756 |
| 32 | 6Pent | 810.28 | 18.661 | 1.47 | 5.917 |
| 33 | 7Hex | 1152.39 | 16.135 | 2.35 | 5.116 |

Beta Glucan (yeast) refers to a polysaccharide with a β-1,4/β1,3 glucose backbone in a 5 to 3 ratio. Beta Glucan (yeast) can be derived from the cell walls of many fungi. The produced oligosaccharides possessed various structural features similar to those in the parent polysaccharide. The composition being 96.37% Glucose (Table 3B). With the glycosidic linkage composition being 35.52% 3-glucose, 21.18% 4-glucose, 18.75% terminal glucose and 14.75% 6-glucose (Table 4B). 21 oligosaccharides were observed in the pool that ranged from 3 hexose to 7 hexoses in length. The most abundant structures represent 3Hex, 4.752 min, 10.738 min and 17.48 min; 4Hex 10.629 min and 24.508 min; 5Hex 13.764 min. The full list of oligosaccharide peaks and abundances are found in Table 23. The oligosaccharide pool can be further distinguished by its 1H-13C 2D-NMR (HSQC) fingerprint (FIG. 13B). Prominent peaks include those described in Table 5B.

TABLE 23
Oligosaccharides generated from the COG depolymerization of Beta
glucan (yeast). Hex refers to hexose sugars, Pent refers to
pentose sugars, HexA refers to hexuronic acid sugars, and Deoxyhex
refers to deoxyhexose sugars. Hexoses refer to glucose.

| | | | RT | Oligo | Retention |
|---|---|---|---|---|---|
| Compound | Identity | Mass | (min) | Wt % | Factor |
| 1 | 3Hex | 504.19 | 4.752 | 7.87 | 1 |
| 2 | 3Hex | 504.19 | 8.159 | 1.79 | 1.717 |
| 3 | 3Hex | 504.19 | 10.738 | 9.68 | 2.26 |
| 4 | 3Hex | 504.19 | 11.389 | 2.16 | 2.397 |
| 5 | 3Hex | 504.19 | 12.883 | 17.48 | 2.711 |
| 6 | 3Hex | 504.19 | 15.154 | 0.63 | 3.189 |
| 7 | 3Hex | 504.19 | 16.363 | 1.92 | 3.443 |
| 8 | 4Hex | 666.24 | 10.629 | 7.59 | 2.237 |
| 9 | 4Hex | 666.24 | 14.611 | 1.01 | 3.075 |
| 10 | 4Hex | 666.24 | 16.655 | 5.87 | 3.505 |
| 11 | 4Hex | 666.24 | 21.124 | 0.85 | 4.445 |
| 12 | 4Hex | 666.24 | 21.668 | 1.62 | 4.56 |
| 13 | 4Hex | 666.24 | 24.508 | 11.40 | 5.157 |
| 14 | 4Hex | 666.24 | 24.804 | 1.71 | 5.22 |
| 15 | 5Hex | 828.29 | 13.764 | 7.65 | 2.896 |
| 16 | 5Hex | 828.29 | 20.094 | 3.59 | 4.229 |
| 17 | 5Hex | 828.29 | 29.904 | 2.84 | 6.293 |
| 18 | 6Hex | 990.34 | 15.62 | 4.97 | 3.287 |
| 19 | 6Hex | 990.34 | 23.157 | 1.83 | 4.873 |
| 20 | 6Hex | 990.34 | 34.633 | 5.89 | 7.288 |
| 21 | 7Hex | 1152.4 | 16.846 | 1.64 | 3.545 |

Tomato peel refers to a natural product polysaccharide arabinogalactan backbone with galacturonic acid, glucose and rhamnose decorations. Tomato peel can be derived from the tomato skin or pomace. The produced oligosaccharides possessed various structural features similar to those in the parent polysaccharide. The arabinose composition being 32.93% followed by 21.80% Galactose, 15.19% galacturonic acid, 13.34% glucose and 11.24% rhamnose (Table 3B). With the glycosidic linkage composition being 29.15% 5-arabinose, 14.55% 4-galactose, 8.69% 2-rhamonose, 7.57% terminal rhamnose, 5.24% terminal glucose and 2.91% 4-glucose (Table 4B). 6 oligosaccharides were observed in the pool that ranged from 3 hexose to 5 hexoses in length. The most abundant structures represent 3Hex, 3.081 min, 4.531 min and 10.081 min; 4Hex 8.191 min and 19.727 min; 5Hex 11.549 min. The full list of oligosaccharide peaks and abundances are found in Table 24. The oligosaccharide pool can be further distinguished by 1H-13C 2D-NMR (HSQC). Prominent peaks include those described in Table 5B.

TABLE 24
Oligosaccharides generated from the COG depolymerization
of Tomato peel. Hex refers to hexose sugars, Pent
refers to pentose sugars, HexA refers to hexuronic
acid sugars, and Deoxyhex refers to deoxyhexose sugars.
Hexoses refer to either glucose or galactose.

| | | | RT | Oligo | Retention |
|---|---|---|---|---|---|
| Compound | Identity | Mass | (min) | Wt % | Factor |
| 1 | 3Hex | 504.19 | 3.081 | 6.11 | 1 |
| 2 | 3Hex | 504.19 | 10.081 | 4.70 | 3.272 |
| 3 | 3Hex (NR) | 504.17 | 4.531 | 75.48 | 1.471 |
| 4 | 4Hex | 666.24 | 8.191 | 5.62 | 2.659 |
| 5 | 4Hex | 666.24 | 19.727 | 3.68 | 6.403 |
| 6 | 5Hex | 828.29 | 11.549 | 4.41 | 3.748 |

Pea fiber refers to a natural product polysaccharide with a α-1,5 arabinose backbone with either α-1,2 or α-1,3 arabinose decorations. Pea fiber can be derived from peas or processes fractions thereof. The produced oligosaccharides possessed various structural features similar to those in the parent polysaccharide. The galactose composition being 79.76% arabinose 9.53% galactose and 4.01% glucose (Table 3B). With the glycosidic linkage composition being 34.99% terminal arabinose, followed by 23.16%5-arabinose, 9.25% 2,5-arabinose, 4.38% 2,3,5, arabinose, 4.54% 4-galactose and 4.38% 2,5-arabinose (Table 4B). 34 oligosaccharides were observed in the pool that ranged from 2 hexose 1 pentose to 6 hexoses 1 pentose in length. The most abundant structures represent 3pent, 6.269 min; 3Hex, 5.023 min and 12.221 min; 3Hex1Pent, 8.958 min; 4Hex 10.829 min and 12.227 min; Hex 13.946 min. The full list of oligosaccharide peaks and abundances are found in Table 25. The oligosaccharide pool can be further distinguished by its 1H-13C 2D-NMR (HSQC) fingerprint (FIG. 13B). Prominent peaks include those described in Table 5B.

TABLE 25
Oligosaccharides generated from the COG depolymerization of
pea fiber. Hex refers to hexose sugars, Pent refers to pentose
sugars, HexA refers to hexuronic acid sugars, and Deoxyhex
refers to deoxyhexose sugar. Pentose refers to arabinose
where hexose refers to either glucose or galactose.

| | | | RT | Oligo | Retention |
|---|---|---|---|---|---|
| Compound | Identity | Mass | (min) | Wt % | Factor |
| 1 | 2Hex1Pent | 474.17 | 3.789 | 3.20 | 1.157 |
| 2 | 2Hex1Pent | 474.17 | 5.019 | 1.67 | 1.532 |
| 3 | 3Hex | 504.18 | 3.276 | 3.64 | 1 |
| 4 | 3Hex | 504.18 | 5.023 | 5.46 | 1.533 |
| 5 | 3Hex | 504.18 | 15.334 | 2.97 | 4.681 |
| 6 | 3Hex (NR) | 504.17 | 3.911 | 1.52 | 1.194 |
| 7 | 3Hex (NR) | 504.17 | 5.613 | 3.36 | 1.713 |
| 8 | 3Hex (NR) | 504.17 | 10.914 | 3.05 | 3.332 |
| 9 | 3Hex (NR) | 504.17 | 12.221 | 8.03 | 3.73 |
| 10 | 3Hex1Pent | 636.23 | 8.958 | 3.67 | 2.734 |
| 11 | 3Hex1Pent | 636.23 | 10.904 | 1.65 | 3.328 |
| 12 | 3Pent | 414.15 | 3.867 | 0.98 | 1.18 |
| 13 | 3Pent | 414.15 | 6.269 | 9.92 | 1.914 |
| 14 | 3Pent | 414.16 | 7.057 | 0.81 | 2.154 |
| 15 | 4Hex | 666.24 | 8.187 | 3.64 | 2.499 |
| 16 | 4Hex | 666.24 | 10.829 | 5.60 | 3.306 |
| 17 | 4Hex | 666.24 | 19.604 | 0.58 | 5.984 |
| 18 | 4Hex | 666.24 | 25.305 | 2.23 | 7.724 |
| 19 | 4Hex (NR) | 666.22 | 9.171 | 3.30 | 2.799 |
| 20 | 4Hex (NR) | 666.22 | 12.226 | 0.95 | 3.732 |
| 21 | 4Hex (NR) | 666.22 | 12.227 | 4.67 | 3.732 |
| 22 | 4Hex (NR) | 666.22 | 15.283 | 3.31 | 4.665 |
| 23 | 4Hex1Pent | 798.28 | 11.988 | 3.05 | 3.659 |
| 24 | 4Hex1Pent | 798.28 | 14.07 | 1.23 | 4.295 |
| 25 | 4Pent | 546.19 | 10.644 | 1.79 | 3.249 |
| 26 | 4Pent | 546.2 | 11.358 | 3.33 | 3.467 |
| 27 | 4Pent | 546.2 | 13.022 | 0.89 | 3.975 |
| 28 | 5Hex | 828.29 | 11.467 | 2.50 | 3.5 |
| 29 | 5Hex | 828.29 | 13.946 | 4.75 | 4.257 |
| 30 | 5Hex | 828.29 | 29.244 | 1.39 | 8.927 |
| 31 | 5Pent | 678.24 | 15.834 | 2.26 | 4.833 |
| 32 | 5Pent | 678.23 | 17.759 | 1.00 | 5.421 |
| 33 | 6Hex | 990.34 | 15.785 | 2.77 | 4.818 |
| 34 | 6Hex1Pent | 1122.39 | 15.078 | 0.83 | 4.603 |

Xantham gum refers to a polysaccharide with a β-1,4 glucose backbone with alternating α-1,2 mannose 33% of the time. Xantham gum can be derived from Xanthamonas campestris. The produced oligosaccharides possessed various structural features similar to those in the parent polysaccharide. The glucose composition being 63.86%, and mannose being 30.64% (Table 3B). With the glycosidic linkage composition being 29.88% 4-glucose, 16.02% 2-mannose, 12.24% 3,6-galactose, 11.54-terminal glucose and 6.09% 4,6-mannose (Table 4B). 61 oligosaccharides were observed in the pool that ranged from 2 hexose 1 pentose to 7 hexoses in length. The most abundant structures represent 3Hex, 4.621 min and 4.969 min; 2Hex1Pent, 4.957 min; 4Hex 8.781 min, 10.45 min, and 10.889 min; 5Hex 12.853 min. The full list of oligosaccharide peaks and abundances are found in Table 26. The oligosaccharide pool can be further distinguished by its 1H-13C 2D-NMR (HSQC) fingerprint (FIG. 13B). Prominent peaks include those described in Table 5B.

TABLE 26
Oligosaccharides generated from the COG depolymerization
of Xanthan Gum. Hex refers to hexose sugars, Pent refers
to pentose sugars, HexA refers to hexuronic acid sugars,
and Deoxyhex refers to deoxyhexose sugars. Pentose refers
to arabinose, Hex A refrers to glucuronic acid and hexose
refers to either glucose or mannose.

| | | | RT | Oligo | Retention |
|---|---|---|---|---|---|
| Compound | Identity | Mass | (min) | Wt % | Factor |
| 1 | 2Hex1HexA | 518.16 | 8.325 | 0.62 | 4.73 |
| 2 | 2Hex1HexA | 518.16 | 8.947 | 3.64 | 5.084 |
| 3 | 2Hex1Pent | 474.17 | 2.959 | 0.17 | 1.681 |
| 4 | 2Hex1Pent | 474.17 | 3.194 | 1.02 | 1.815 |
| 5 | 2Hex1Pent | 474.17 | 4.957 | 3.69 | 2.816 |
| 6 | 2Hex1Pent | 474.17 | 6.228 | 0.45 | 3.539 |
| 7 | 2Hex1Pent | 474.17 | 7.324 | 0.47 | 4.161 |
| 8 | 2Hex1Pent | 474.17 | 8.744 | 0.56 | 4.968 |
| 9 | 3Hex | 504.18 | 1.76 | 0.23 | 1 |
| 10 | 3Hex | 504.18 | 2.979 | 1.35 | 1.693 |
| 11 | 3Hex | 504.18 | 3.493 | 3.1 | 1.985 |
| 12 | 3Hex | 504.18 | 4.368 | 0.71 | 2.482 |
| 13 | 3Hex | 504.18 | 4.621 | 5.04 | 2.626 |
| 14 | 3Hex | 504.19 | 4.969 | 10.34 | 2.823 |
| 15 | 3Hex | 504.18 | 6.283 | 1.47 | 3.57 |
| 16 | 3Hex | 504.18 | 7.081 | 1.01 | 4.023 |
| 17 | 3Hex | 504.18 | 9.241 | 1.5 | 5.251 |
| 18 | 3Hex | 504.18 | 9.776 | 2.12 | 5.555 |
| 19 | 3Hex | 504.18 | 15.365 | 1.46 | 8.73 |
| 20 | 3Hex1HexA | 680.22 | 4.082 | 0.5 | 2.319 |
| 21 | 3Hex1HexA | 680.22 | 6.239 | 0.48 | 3.545 |
| 22 | 3Hex1HexA | 680.22 | 19.397 | 2.04 | 11.021 |
| 23 | 3Hex1Pent | 636.23 | 7.418 | 0.22 | 4.215 |
| 24 | 3Hex1Pent | 636.23 | 8.142 | 3.1 | 4.626 |
| 25 | 3Hex1Pent | 636.23 | 9.592 | 1.44 | 5.45 |
| 26 | 3Hex1Pent | 636.23 | 10.399 | 1.06 | 5.909 |
| 27 | 3Hex1Pent | 636.23 | 10.963 | 1.67 | 6.229 |
| 28 | 3Hex1Pent | 636.23 | 16.021 | 0.65 | 9.103 |
| 29 | 4Hex | 666.24 | 7.534 | 1.63 | 4.281 |
| 30 | 4Hex | 666.24 | 7.918 | 0.6 | 4.499 |
| 31 | 4Hex | 666.24 | 8.781 | 7.65 | 4.989 |
| 32 | 4Hex | 666.24 | 9.533 | 3.13 | 5.416 |
| 33 | 4Hex | 666.24 | 10.45 | 4.42 | 5.938 |
| 34 | 4Hex | 666.24 | 10.889 | 3.98 | 6.187 |
| 35 | 4Hex | 666.24 | 11.44 | 0.5 | 6.5 |
| 36 | 4Hex | 666.24 | 11.7 | 0.56 | 6.648 |
| 37 | 4Hex | 666.24 | 12.426 | 1.56 | 7.06 |
| 38 | 4Hex | 666.24 | 16.009 | 0.45 | 9.096 |
| 39 | 4Hex | 666.24 | 16.252 | 1.35 | 9.234 |
| 40 | 4Hex | 666.24 | 25.297 | 0.23 | 14.373 |
| 41 | 4Hex1HexA | 842.27 | 8.233 | 0.65 | 4.678 |
| 42 | 4Hex1HexA | 842.27 | 13.575 | 1.24 | 7.713 |
| 43 | 4Hex1Pent | 798.28 | 11.859 | 0.58 | 6.738 |
| 44 | 4Hex1Pent | 798.28 | 12.178 | 0.74 | 6.919 |
| 45 | 4Hex1Pent | 798.28 | 13.095 | 2.49 | 7.44 |
| 46 | 4Hex1Pent | 798.28 | 14.144 | 0.79 | 8.036 |
| 47 | 4Hex1Pent | 798.28 | 19.536 | 0.42 | 11.1 |
| 48 | 5Hex | 828.29 | 11.298 | 0.55 | 6.419 |
| 49 | 5Hex | 828.29 | 11.761 | 0.51 | 6.682 |
| 50 | 5Hex | 828.29 | 11.962 | 2.04 | 6.797 |
| 51 | 5Hex | 828.29 | 12.608 | 1.79 | 7.164 |
| 52 | 5Hex | 828.29 | 12.853 | 5.08 | 7.303 |
| 53 | 5Hex | 828.29 | 13.744 | 0.63 | 7.809 |
| 54 | 5Hex | 828.29 | 14.038 | 2.43 | 7.976 |
| 55 | 5Hex | 828.29 | 15.144 | 0.39 | 8.605 |
| 56 | 5Hex | 828.29 | 17.108 | 0.41 | 9.72 |
| 57 | 5Hex | 828.29 | 19.354 | 0.82 | 10.997 |
| 58 | 5Hex | 828.29 | 21.535 | 0.24 | 12.236 |
| 59 | 5Hex1Pent | 960.33 | 15.57 | 0.28 | 8.847 |
| 60 | 6Hex | 990.34 | 15.31 | 0.93 | 8.699 |
| 61 | 7Hex | 1152.39 | 16.851 | 0.77 | 9.574 |

Spent coffee grounds refer to a natural product that is high in fiber, the oligosaccharides we produced matched this composition. Spent coffee ground can be derived from coffee cherries or processed fractions thereof. The galactose composition being 49.81%, followed by mannose, and arabinose at 25.99%, and 14.03% respectively (Table 3B). With the glycosidic linkage composition being 22.84% 4-mannose, 15.54 terminal arabinose, 9.37% 3-galactose, 9.13%-terminal galactose and 8.86%-terminal mannose (Table 4B). 38 oligosaccharides were observed in the pool that ranged from 1 hexose and 2 pentose to 7 hexoses in length. The most abundant structures represent 3Hex, 1.612 min; 2Hex1Pent, 2.556 min; 4Hex 4.466 min; 3Hex1Pent 7.337 min; 4Hex1pent, 10.802 min; 6Hex, 12.239 min 2; Hex1Pent, 13.533 min. The full list of oligosaccharide peaks and abundances are found in Table 27. The full list of oligosaccharides can be further distinguished by its 1H-13C 2D-NMR (HSQC) fingerprint (FIG. 13B). Prominent peaks include those described in Table 5B.

TABLE 27
Oligosaccharides generated from the COG depolymerization of Spent
Coffee Grounds. Hex refers to hexose sugars, Pent refers to
pentose sugars, HexA refers to hexuronic acid sugars, and Deoxyhex
refers to deoxyhexose sugars. Hexoses refer to mannose, glucose,
or galactose, and pentose refers to arabinose.

| | | | RT | Oligo | Retention |
|---|---|---|---|---|---|
| Compound | Identity | Mass | (min) | Wt % | Factor |
| 1 | 1Hex2Pent | 444.16 | 3.49 | 2.79 | 2.165 |
| 2 | 1Hex2Pent | 444.16 | 4.245 | 0.86 | 2.633 |
| 3 | 2Hex1Pent | 474.17 | 2.556 | 6.88 | 1.586 |
| 4 | 2Hex1Pent | 474.18 | 3.526 | 0.98 | 2.187 |
| 5 | 2Hex2Pent | 606.22 | 9.748 | 1.92 | 6.047 |
| 6 | 3Hex | 504.18 | 1.612 | 7.96 | 1 |
| 7 | 3Hex | 504.18 | 1.893 | 1.51 | 1.174 |
| 8 | 3Hex | 504.18 | 2.501 | 0.61 | 1.551 |
| 9 | 3Hex | 504.18 | 2.914 | 1.51 | 1.808 |
| 10 | 3Hex | 504.18 | 6.515 | 3.95 | 4.042 |
| 11 | 3Hex | 504.18 | 15.334 | 0.51 | 9.512 |
| 12 | 3Hex (NR) | 504.17 | 3.977 | 3.31 | 2.467 |
| 13 | 3Hex1Pent | 636.23 | 7.337 | 11.63 | 4.551 |
| 14 | 3Hex1Pent | 636.23 | 7.99 | 0.32 | 4.957 |
| 15 | 3Hex1Pent | 636.23 | 11.063 | 0.67 | 6.863 |
| 16 | 3Hex1Pent | 636.23 | 12.762 | 0.52 | 7.917 |
| 17 | 3Hex1Pent | 636.23 | 17.198 | 0.35 | 10.669 |
| 18 | 3Hex1Pent | 636.22 | 18.732 | 0.33 | 11.62 |
| 19 | 3Hex2Pent | 768.27 | 12.108 | 1.25 | 7.511 |
| 20 | 4Hex | 666.24 | 4.466 | 16.37 | 2.77 |
| 21 | 4Hex | 666.24 | 6.189 | 0.36 | 3.839 |
| 22 | 4Hex | 666.24 | 7.072 | 1.02 | 4.387 |
| 23 | 4Hex | 666.24 | 7.779 | 1.14 | 4.826 |
| 24 | 4Hex | 666.24 | 10.083 | 1.37 | 6.255 |
| 25 | 4Hex | 666.24 | 11.392 | 0.63 | 7.067 |
| 26 | 4Hex | 666.24 | 16.026 | 1.28 | 9.942 |
| 27 | 4Hex | 666.24 | 17.584 | 0.38 | 10.908 |
| 28 | 4Hex | 666.24 | 25.259 | 0.41 | 15.669 |
| 29 | 4Hex1Pent | 798.28 | 10.802 | 8.61 | 6.701 |
| 30 | 4Hex1Pent | 798.28 | 17.816 | 0.49 | 11.052 |
| 31 | 5Hex | 828.29 | 8.774 | 2.19 | 5.443 |
| 32 | 5Hex | 828.29 | 16.339 | 1.0 | 10.136 |
| 33 | 5Hex | 828.29 | 22.876 | 0.72 | 14.191 |
| 34 | 5Hex1Pent | 960.33 | 13.533 | 4.45 | 8.395 |
| 35 | 6Hex | 990.34 | 12.239 | 6.61 | 7.592 |
| 36 | 6Hex | 990.34 | 13.162 | 0.5 | 8.165 |
| 37 | 6Hex1Pent | 1122.38 | 15.771 | 1.91 | 9.783 |
| 38 | 7Hex | 1152.39 | 14.381 | 2.7 | 8.921 |

Gellan Gum refers to a polysaccharide with a backbone of β-1,4 glucose and α-1,3 rhamnose in a 3 to 1 ratio respectively. Gellan gum can be derived from Sphingomonas elodea. The produced oligosaccharides possessed various structural features similar to those in the parent polysaccharide. The glucose composition being 44.29%, followed by 42.94% rhamnose and 9.70% galacturonic acid (Table 3B). With the glycosidic linkage composition being 4-glucose at 24.24%, 4-rhamnose at 21.57%, 3-glucose at 20.96% terminal glucose at 10.25% and terminal rhamnose at 7.33% (Table 4B). 19 oligosaccharides were observed in the pool that ranged from 2 hexose and 1 Deoxyhexose to 7 hexose in length. The most abundant structures represent 2Hex1HexA1Deoxyhex, 17.01 min, 26.202 min, 30.461 min and 34.83 min; 1HexHexA1Deoxyhex, 19.054 min; 3Hex1HexA1Deoxyhex, 28.498 min and 34.83 min. The full list of oligosaccharide peaks and abundances are found in Table 28. The oligosaccharide pool can be further distinguished by it's 1H-13C 2D-NMR (HSQC) fingerprint (FIG. 13B). Prominent peaks include those described in Table 5B.

TABLE 28
Oligosaccharides generated from the COG depolymerization
of Gellan Gum. Hex refers to hexose sugars, Pent refers
to pentose sugars, HexA refers to hexuronic acid sugars,
and Deoxyhex refers to deoxyhexose sugars. Where deoxyhexose
is rhamnose and hexose is glucose.

| Com- | | | RT | Oligo | Retention |
|---|---|---|---|---|---|
| pound | Identity | Mass | (min) | Wt % | Factor |
| 1 | 1Hex1HexA1Deoxyhex | 502.17 | 19.054 | 10.75 | 7.46 |
| 2 | 2Hex1Deoxyhex | 488.19 | 2.554 | 5.22 | 1 |
| 3 | 2Hex1HexA | 518.16 | 18.245 | 6.09 | 7.144 |
| 4 | 2Hex1HexA1Deoxyhex | 664.22 | 17.01 | 20.06 | 6.66 |
| 5 | 2Hex1HexA1Deoxyhex | 664.22 | 26.202 | 11.78 | 10.259 |
| 6 | 2Hex1HexA1Deoxyhex | 664.22 | 30.461 | 6.73 | 11.927 |
| 7 | 2Hex1HexA2Deoxyhex | 810.28 | 34.83 | 7.10 | 13.637 |
| 8 | 3Hex | 504.19 | 3.133 | 0.21 | 1.227 |
| 9 | 3Hex | 504.19 | 3.384 | 0.24 | 1.325 |
| 10 | 3Hex | 504.18 | 4.804 | 2.33 | 1.881 |
| 11 | 3Hex1HexA1Deoxyhex | 826.28 | 28.498 | 11.38 | 11.158 |
| 12 | 3Hex1HexA1Deoxyhex | 826.27 | 28.523 | 3.44 | 11.168 |
| 13 | 3Hex1HexA2Deoxyhex | 972.33 | 32.07 | 8.64 | 12.557 |
| 14 | 4Hex | 666.24 | 9.489 | 0.27 | 3.715 |
| 15 | 4Hex | 666.24 | 10.444 | 0.32 | 4.089 |
| 16 | 4Hex | 666.24 | 10.847 | 2.00 | 4.247 |
| 17 | 4Hex | 666.23 | 16.998 | 1.52 | 6.655 |
| 18 | 5Hex | 828.29 | 13.948 | 1.36 | 5.461 |
| 19 | 8Hex | 1314.46 | 26.197 | 0.55 | 10.257 |

Soy Fiber refers to a natural product that is high in fiber. Soy fiber can be derived from soy or processed fractions thereof. The produced oligosaccharides possessed various structural features similar to those in the parent polysaccharide. The galactose composition being 49.61%, followed by arabinose, xylose and glucose at 34.07%, 4.82% and 2.78% respectively (Table 3B). With the glycosidic linkage composition being 4-galactose at 41.54%, terminal arabinose at 13.98%, 5-arabinose at 11.11%, terminal galactose at 5.28% and 3,5-arbinose at 4.81% (Table 4B). 30 oligosaccharides were observed in the pool that ranged from 3 hexose and a pentose to 7 hexoses and a pentose in length. The most abundant structures represent 3Hex, 3.24 min; 2Hex1Pent, 3.759 min; 4Hex 8.283 min; 3Hex1Pent, 9.023 min; Hex 11.557 min; 4Hex1Pent, 12.045 min; 5HexPent, 13.904 min. The full list of oligosaccharide peaks and abundances are found in Table 29. The oligosaccharide pool can be further distinguished by it's 1H-13C 2D-NMR (HSQC) fingerprint (FIG. 13B). Prominent peaks include those described in Table 5B.

TABLE 29
Oligosaccharides generated from the COG depolymerization
of Soy fiber. Hex refers to hexose sugars, Pent refers
to pentose sugars, HexA refers to hexuronic acid sugars,
and Deoxyhex refers to deoxyhexose sugars. Where HexA
is galacturonic acid, Hexose is glucose or galactose
and pentose is either arabinose or xylose.

| | | | RT | Oligo | Retention |
|---|---|---|---|---|---|
| Compound | Identity | Mass | (min) | Wt % | Factor |
| 1 | 2Hex1HexA | 518.16 | 5.195 | 2.97 | 1.612 |
| 2 | 2Hex1Pent | 474.17 | 3.223 | 2.41 | 1 |
| 3 | 2Hex1Pent | 474.17 | 3.759 | 8.13 | 1.166 |
| 4 | 2Hex2Pent | 606.22 | 8.304 | 1.36 | 2.576 |
| 5 | 2Hex2Pent | 606.22 | 9.147 | 2.01 | 2.838 |
| 6 | 3Hex | 504.18 | 3.24 | 8.55 | 1.005 |
| 7 | 3Hex | 504.18 | 5.024 | 1.54 | 1.559 |
| 8 | 3Hex1HexA | 680.22 | 10.517 | 5.15 | 3.263 |
| 9 | 3Hex1Pent | 636.23 | 7.456 | 0.70 | 2.313 |
| 10 | 3Hex1Pent | 636.22 | 8.003 | 0.83 | 2.483 |
| 11 | 3Hex1Pent | 636.23 | 8.309 | 2.00 | 2.578 |
| 12 | 3Hex1Pent | 636.23 | 9.023 | 9.78 | 2.8 |
| 13 | 3Hex2Pent | 768.27 | 11.553 | 0.98 | 3.585 |
| 14 | 3Hex2Pent | 768.27 | 11.749 | 0.64 | 3.645 |
| 15 | 3Hex2Pent | 768.27 | 12.553 | 1.84 | 3.895 |
| 16 | 4Hex | 666.24 | 8.283 | 9.35 | 2.57 |
| 17 | 4Hex | 666.24 | 10.913 | 1.47 | 3.386 |
| 18 | 4Hex | 666.24 | 19.608 | 0.38 | 6.084 |
| 19 | 4Hex1HexA | 842.27 | 13.588 | 4.66 | 4.216 |
| 20 | 4Hex1Pent | 798.28 | 10.957 | 0.49 | 3.4 |
| 21 | 4Hex1Pent | 798.28 | 11.271 | 1.01 | 3.497 |
| 22 | 4Hex1Pent | 798.28 | 12.045 | 9.22 | 3.737 |
| 23 | 4Hex2Pent | 930.32 | 13.503 | 0.42 | 4.19 |
| 24 | 5Hex | 828.29 | 11.557 | 6.93 | 3.586 |
| 25 | 5Hex1HexA | 1004.32 | 15.435 | 3.39 | 4.789 |
| 26 | 5Hex1Pent | 960.33 | 13.319 | 0.89 | 4.132 |
| 27 | 5Hex1Pent | 960.33 | 13.904 | 5.94 | 4.314 |
| 28 | 6Hex | 990.34 | 13.511 | 4.43 | 4.192 |
| 29 | 6Hex1HexA | 1166.38 | 16.633 | 1.45 | 5.161 |
| 30 | 7Hex1Pent | 1284.44 | 16.31 | 1.09 | 5.061 |

Carrot Fiber refers to a natural product that is high in fiber with predominately an arabinogalactan structure. Carrot fiber can be derived from carrot or processed fractions thereof. The produced oligosaccharides possessed various structural features similar to those in the parent polysaccharide. The arabinose composition being 56.85%, followed by galactose at 25.82% and glucose at 13.58% (Table 3B). With the glycosidic linkage composition being 5-arabinose at 18.64%, 4-galactose at 17.73%, 3,5-arbinose at 7.89% and terminal glucose at 5.25% (Table 4B). 33 oligosaccharides were observed in the pool that ranged from 2 hexose a pentose to 8 hexoses in length. The most abundant structures represent 3Hex, 3.144 min; 2Hex1Pent, 3.655 min; 4Hex 8.181 min; 3Hex1Pent, 8.969 min; 5Hex 11.489 min; 4Hex1Pent, 11.989 min; 6Hex, 13.482 min. The full list of oligosaccharide peaks and abundances are found in Table 30. The oligosaccharide pool can be further distinguished by 1H-13C 2D-NMR (HSQC). Prominent peaks include those described in Table 5B.

TABLE 30
Oligosaccharides generated from the COG depolymerization
of Carrot fiber. Hex refers to hexose sugars, Pent refers
to pentose sugars, HexA refers to hexuronic acid sugars,
and Deoxyhex refers to deoxyhexose sugars. Where pentose
is arabinose, HexA is glucuronic acid or galacturonic
acid and hexose is either galactose or glucose.

| | | | RT | Oligo | Retention |
|---|---|---|---|---|---|
| Compound | Identity | Mass | (min) | Wt % | Factor |
| 1 | 2Hex1HexA | 518.16 | 4.847 | 4.08 | 1.542 |
| 2 | 2Hex1Pent | 474.17 | 3.158 | 3.15 | 1.004 |
| 3 | 2Hex1Pent | 474.17 | 3.655 | 6.39 | 1.163 |
| 4 | 2Hex2Pent | 606.22 | 9.104 | 1.54 | 2.896 |
| 5 | 3Hex | 504.19 | 3.144 | 13.45 | 1 |
| 6 | 3Hex | 504.18 | 4.11 | 0.64 | 1.307 |
| 7 | 3Hex | 504.19 | 4.746 | 1.17 | 1.51 |
| 8 | 3Hex | 504.18 | 8.699 | 0.7 | 2.767 |
| 9 | 3Hex | 504.18 | 10.063 | 1.05 | 3.201 |
| 10 | 3Hex | 504.18 | 15.314 | 1.75 | 4.871 |
| 11 | 3Hex1HexA | 680.22 | 10.322 | 3.86 | 3.283 |
| 12 | 3Hex1Pent | 636.23 | 8.218 | 3.22 | 2.614 |
| 13 | 3Hex1Pent | 636.23 | 8.969 | 6.63 | 2.853 |
| 14 | 3Hex2Pent | 768.27 | 12.501 | 1.43 | 3.976 |
| 15 | 4Hex | 666.24 | 8.181 | 13.39 | 2.602 |
| 16 | 4Hex | 666.24 | 9.36 | 0.56 | 2.977 |
| 17 | 4Hex | 666.24 | 10.786 | 1.12 | 3.431 |
| 18 | 4Hex | 666.24 | 19.573 | 0.65 | 6.226 |
| 19 | 4Hex | 666.24 | 20.122 | 0.56 | 6.4 |
| 20 | 4Hex | 666.24 | 25.25 | 1.12 | 8.031 |
| 21 | 4Hex1HexA | 842.27 | 13.425 | 2.57 | 4.27 |
| 22 | 4Hex1Pent | 798.28 | 11.989 | 7.33 | 3.813 |
| 23 | 4Hex2Pent | 930.32 | 14.282 | 0.84 | 4.543 |
| 24 | 5Hex | 828.29 | 11.489 | 9.34 | 3.654 |
| 25 | 5Hex | 828.29 | 12.209 | 0.4 | 3.883 |
| 26 | 5Hex | 828.29 | 26.348 | 0.39 | 8.38 |
| 27 | 5Hex | 828.29 | 27.127 | 0.31 | 8.628 |
| 28 | 5Hex | 828.29 | 29.233 | 0.75 | 9.298 |
| 29 | 5Hex1Pent | 960.33 | 13.887 | 3.62 | 4.417 |
| 30 | 6Hex | 990.34 | 13.482 | 4.8 | 4.288 |
| 31 | 6Hex1Pent | 1122.38 | 15.142 | 1.9 | 4.816 |
| 32 | 7Hex | 1152.39 | 14.813 | 0.62 | 4.712 |
| 33 | 8Hex | 1314.45 | 15.9 | 0.66 | 5.057 |

Sugar Cane Fiber refers to a natural product that is high in fiber with predominately an xylan structure. Sugar cane fiber can be derived from sugar cane or processed fractions thereof. The produced oligosaccharides possessed various structural features similar to those in the parent polysaccharide. The xylose composition being 47.48%, followed by glucose at 35.32% and 12.24% arabinose (Table 3B). With the glycosidic linkage composition being 4-xylose at 27.17%, 4-glucose at 15.54%, terminal glucose at 8.94%, terminal xylose at 8.31% and terminal arabinose at 5.94% (Table 4B). 33 oligosaccharides were observed in the pool that ranged from 3 hexose and a pentose to 6 hexoses in length. The most abundant structures represent 3Pent, 9.736 min; 2Hex1Pent, 13.955 min; 3Hex, 15.414 min 2; 4Pent, 18.132 min; 4Hex, 25.37 min; 5Hex, 29.329 min. The full list of oligosaccharide peaks and abundances are found in Table 31. The oligosaccharide pool can be further distinguished by 1H-13C 2D-NMR (HSQC). Prominent peaks include those described in Table 5B.

TABLE 31
Oligosaccharides generated from the COG depolymerization of
sugar cane fiber. Hex refers to hexose sugars, Pent refers
to pentose sugars, HexA refers to hexuronic acid sugars, and
Deoxyhex refers to deoxyhexose sugars. Where hexose is glucose
or galactose and pentose is either xylose or arabinose.

| | | | RT | Oligo | Retention |
|---|---|---|---|---|---|
| Compound | Identity | Mass | (min) | Wt % | Factor |
| 1 | 2Hex1Pent | 474.18 | 13.955 | 3.91 | 7.131 |
| 2 | 3Hex | 504.18 | 11.599 | 1.85 | 5.927 |
| 3 | 3Hex | 504.19 | 15.414 | 18.53 | 7.876 |
| 4 | 3Hex | 504.18 | 18.158 | 2.07 | 9.278 |
| 5 | 3Hex (NR) | 504.17 | 1.957 | 1.30 | 1 |
| 6 | 3Hex (NR) | 504.17 | 3.84 | 0.30 | 1.962 |
| 7 | 3Hex (NR) | 504.17 | 5.404 | 1.29 | 2.761 |
| 8 | 3Hex1Pent | 636.23 | 24.526 | 2.88 | 12.532 |
| 9 | 3Hex1Pent | 636.23 | 25.874 | 0.31 | 13.221 |
| 10 | 3Pent | 414.15 | 2.398 | 0.39 | 1.225 |
| 11 | 3Pent | 414.15 | 9.736 | 8.48 | 4.975 |
| 12 | 3Pent | 414.15 | 13.843 | 0.82 | 7.074 |
| 13 | 4Hex | 666.24 | 21.972 | 1.80 | 11.227 |
| 14 | 4Hex | 666.24 | 25.37 | 13.78 | 12.964 |
| 15 | 4Hex | 666.24 | 25.62 | 2.34 | 13.091 |
| 16 | 4Hex | 666.24 | 26.463 | 2.28 | 13.522 |
| 17 | 4Hex1Pent | 798.28 | 29.11 | 1.70 | 14.875 |
| 18 | 4Pent | 546.19 | 10.029 | 1.13 | 5.125 |
| 19 | 4Pent | 546.2 | 15.641 | 1.48 | 7.992 |
| 20 | 4Pent | 546.19 | 18.132 | 7.52 | 9.265 |
| 21 | 4Pent | 546.2 | 21.384 | 0.76 | 10.927 |
| 22 | 5Hex | 828.29 | 28.523 | 0.74 | 14.575 |
| 23 | 5Hex | 828.29 | 29.329 | 8.52 | 14.987 |
| 24 | 5Hex | 828.29 | 29.834 | 1.76 | 15.245 |
| 25 | 5Hex | 828.29 | 30.473 | 2.21 | 15.571 |
| 26 | 5Pent | 678.24 | 17.349 | 0.70 | 8.865 |
| 27 | 5Pent | 678.24 | 20.908 | 0.79 | 10.684 |
| 28 | 5Pent | 678.24 | 21.722 | 1.17 | 11.1 |
| 29 | 5Pent | 678.24 | 24.34 | 5.23 | 12.437 |
| 30 | 5Pent | 678.24 | 26.576 | 0.50 | 13.58 |
| 31 | 6Hex | 990.34 | 33.38 | 0.47 | 17.057 |
| 32 | 6Pent | 810.28 | 27.583 | 1.49 | 14.095 |
| 33 | 7Pent | 942.32 | 29.641 | 1.51 | 15.146 |

Pacific kelp powder refers to a natural product that is milled from dried kelp. Pacific kelp powder can be derived from Phaeophyta or other brown algae. The oligosaccharides were comprised of 55.35% fucose, 16.21% xylose and 13.04% glucose. (Table 3B). The glycosidic linkage composition comprised 14.16% terminal xylose, 13.71% terminal fucose, 10.9% terminal glucose, 10.16% 3-glucose and 5.87% 4-xylose(Table 4B). 9 oligosaccharides were observed in the pool that ranged from 3 hexose to 12 hexoses in length. The most abundant structures represent 3Hex, 11.17 min, 11.66 min and 13.234 min; 4Hex 15.979 min and 25.316 min; 5Hex 20.596 min and 30.299 min. The full list of oligosaccharide peaks and abundances are found in Table 32. The oligosaccharide pool can be further distinguished by 1H-13C 2D-NMR (HSQC). Prominent peaks include those described in Table 5B.

TABLE 32
Oligosaccharides generated from the COG depolymerization of Pacific
kelp powder. Hex refers to hexose sugars, Pent refers to pentose
sugars, HexA refers to hexuronic acid sugars, and Deoxyhex refers
to deoxyhexose sugars. Hexose refers to glucose or galactose.

| | | | RT | Oligo | Retention |
|---|---|---|---|---|---|
| Compound | Identity | Mass | (min) | Wt % | Factor |
| 1 | 3Hex | 504.19 | 10.311 | 1.54 | 1 |
| 2 | 3Hex | 504.19 | 11.17 | 57.93 | 1.083 |
| 3 | 3Hex | 504.19 | 11.66 | 1.76 | 1.131 |
| 4 | 3Hex | 504.19 | 13.234 | 7.75 | 1.283 |
| 5 | 4Hex | 666.24 | 15.979 | 16.91 | 1.55 |
| 6 | 4Hex | 666.24 | 24.101 | 1.69 | 2.337 |
| 7 | 4Hex | 666.24 | 25.316 | 5.69 | 2.455 |
| 8 | 5Hex | 828.29 | 20.596 | 3.7 | 1.997 |
| 9 | 5Hex | 828.29 | 30.229 | 3.02 | 2.932 |

Sea lettuce powder refers to a natural product that is milled from dried members of the Ulva genus. The oligosaccharides were comprised of 62.82% arabinose, 13.95% xylose and 13.46% glucose. (Table 3B). The glycosidic linkage composition comprised 46.48% 4-glucose, 15.29% terminal glucose, 7.39% 4-xylose and 5.19% 2-mannose (Table 4B). 9 oligosaccharides were observed in the pool that ranged from 2 hexose and 1 pentose to 4 hexoses in length. The most abundant structures represent 3Hex, 5.29 min, 11.691 min, 15.43 min and 18.209 min; 4Hex 10.99 min and 25.492 min; 2Hex1pent 13.343 min. The full list of oligosaccharide peaks and abundances are found in Table 33. The oligosaccharide pool can be further distinguished by its 1H-13C 2D-NMR (HSQC). Prominent peaks include those described in Table 5B.

TABLE 33
Oligosaccharides generated from the COG depolymerization of
Pacific kelp powder. Hex refers to hexose sugars, Pent refers
to pentose sugars, HexA refers to hexuronic acid sugars,
and Deoxyhex refers to deoxyhexose sugars. Hexose refers
to glucose and pentose refers to arabinose or xylose.

| | | | RT | Oligo | Retention |
|---|---|---|---|---|---|
| Compound | Identity | Mass | (min) | Wt % | Factor |
| 1 | 2Hex1Pent | 474.18 | 12.989 | 4.57 | 2.455 |
| 2 | 2Hex1Pent | 474.18 | 13.343 | 12.15 | 2.522 |
| 3 | 2Hex1Pent | 474.17 | 15.42 | 4.01 | 2.915 |
| 4 | 3Hex | 504.19 | 5.29 | 20.31 | 1 |
| 5 | 3Hex | 504.18 | 11.691 | 8.32 | 2.21 |
| 6 | 3Hex | 504.19 | 15.43 | 23.71 | 2.917 |
| 7 | 3Hex | 504.18 | 18.209 | 8.52 | 3.442 |
| 8 | 4Hex | 666.24 | 10.999 | 7.77 | 2.079 |
| 9 | 4Hex | 666.24 | 25.492 | 10.65 | 4.819 |

Olive liquid waste refers to a natural product that is high in fiber with predominately an xyloglucan structure. Olive liquid waste can refer to olives or processed fractions thereof. The produced oligosaccharides possessed various structural features similar to those in the parent polysaccharide. The arabinose composition being 55.25%, followed by galactose at 13.48%, glucose at 11.55% and 6.19% rhamnose (Table 3B). With the glycosidic linkage composition being terminal arabinose at 21.67%, followed by 20.97% 5-arabinose, 13.51% 3,5-arabinose, 4.73% 4-galactose and 4.32% terminal glucose (Table 4B). The oligosaccharide pool can be further distinguished by 1H-13C 2D-NMR (HSQC). Prominent peaks include those described in Table 5B.

Beet pectin refers to a natural product that is high in fiber. Beet pectin can be derived from beets or processed fractions thereof. The produced oligosaccharides possessed various structural features similar to those in the parent polysaccharide. The galactose composition being 53.14%, followed by arabinose at 14.59%, galacturonic acid at 13.78% and rhamnose at 11.53% (Table 3B). With the glycosidic linkage composition being 4-galactose at 26.45%, followed by 11.19% terminal galactose, 9.53% 2-rhamnose, 7% terminal arabinose and 6% 6-galactose (Table 4B). 47 oligosaccharides were observed in the pool that ranged from 1 hexose and a pentose to 7 hexoses in length. The most abundant structures represent 3Hex, 3.173 min; 2Hex1Pent 3.709 min and 4.815 min; 3Hex1Pent, 7.423 min and 8.997 min; 4Hex 8.239 min; 4Hex1Pent, 12.022 min. The full list of oligosaccharide peaks and abundances are found in Table 34. The oligosaccharide pool can be further distinguished by 1H-13C 2D-NMR (HSQC). Prominent peaks include those described in Table 5B.

TABLE 34
Oligosaccharides generated from the COG depolymerization of
beet pectin. Hex refers to hexose sugars, Pent refers to pentose
sugars, HexA refers to hexuronic acid sugars, and Deoxyhex
refers to deoxyhexose sugars. Where Hexose refers to either
glucose or galactose, and pentose refers to arabinose.

| | | | RT | Oligo | Retention |
|---|---|---|---|---|---|
| Compound | Identity | Mass | (min) | Wt % | Factor |
| 1 | 1Hex2Pent | 444.16 | 3.718 | 0.75 | 1.172 |
| 2 | 1Hex2Pent | 444.16 | 4.377 | 0.83 | 1.379 |
| 3 | 1Hex2Pent | 444.16 | 4.632 | 1.51 | 1.46 |
| 4 | 1Hex2Pent | 444.16 | 12.001 | 0.29 | 3.782 |
| 5 | 2Hex1Pent | 474.17 | 3.188 | 0.49 | 1.005 |
| 6 | 2Hex1Pent | 474.17 | 3.709 | 7.77 | 1.169 |
| 7 | 2Hex1Pent | 474.17 | 3.716 | 0.53 | 1.171 |
| 8 | 2Hex1Pent | 474.17 | 4.815 | 5.84 | 1.517 |
| 9 | 2Hex1Pent | 474.17 | 10.775 | 0.97 | 3.396 |
| 10 | 2Hex2Pent | 606.22 | 9.138 | 0.77 | 2.88 |
| 11 | 2Hex2Pent | 606.21 | 9.924 | 0.41 | 3.128 |
| 12 | 2Hex2Pent | 606.21 | 15.467 | 0.56 | 4.875 |
| 13 | 2Hex2Pent | 606.22 | 16.12 | 0.47 | 5.08 |
| 14 | 2Hex2Pent | 606.22 | 16.855 | 0.82 | 5.312 |
| 15 | 2Hex2Pent | 606.22 | 18.948 | 0.19 | 5.972 |
| 16 | 3Hex | 504.18 | 3.173 | 6.87 | 1 |
| 17 | 3Hex | 504.18 | 4.188 | 0.21 | 1.32 |
| 18 | 3Hex | 504.18 | 4.815 | 0.89 | 1.517 |
| 19 | 3Hex | 504.18 | 6.169 | 5.69 | 1.944 |
| 20 | 3Hex (NR) | 504.17 | 9.422 | 0.72 | 2.969 |
| 21 | 3Hex1Pent | 636.23 | 7.423 | 7.34 | 2.339 |
| 22 | 3Hex1Pent | 636.23 | 8.283 | 0.53 | 2.61 |
| 23 | 3Hex1Pent | 636.23 | 8.997 | 9.54 | 2.835 |
| 24 | 3Hex1Pent | 636.23 | 10.878 | 0.53 | 3.428 |
| 25 | 3Hex1Pent | 636.23 | 17.498 | 0.85 | 5.515 |
| 26 | 3Hex1Pent | 636.23 | 18.194 | 0.3 | 5.734 |
| 27 | 3Hex1Pent | 636.23 | 18.542 | 0.3 | 5.844 |
| 28 | 3Hex2Pent | 768.27 | 12.532 | 0.88 | 3.95 |
| 29 | 3Hex2Pent | 768.27 | 15.417 | 0.89 | 4.859 |
| 30 | 3Hex2Pent | 768.27 | 19.189 | 0.72 | 6.048 |
| 31 | 3Hex2Pent | 768.27 | 21.439 | 0.24 | 6.757 |
| 32 | 4Hex | 666.24 | 6.403 | 4.75 | 2.018 |
| 33 | 4Hex | 666.24 | 8.239 | 6.94 | 2.597 |
| 34 | 4Hex | 666.24 | 10.798 | 1.04 | 3.403 |
| 35 | 4Hex | 666.24 | 17.659 | 0.32 | 5.565 |
| 36 | 4Hex1Pent | 798.28 | 11.58 | 3.05 | 3.65 |
| 37 | 4Hex1Pent | 798.28 | 12.022 | 7.07 | 3.789 |
| 38 | 4Hex1Pent | 798.28 | 13.995 | 0.52 | 4.411 |
| 39 | 4Hex1Pent | 798.28 | 14.492 | 0.65 | 4.567 |
| 40 | 5Hex | 828.29 | 10.988 | 1.62 | 3.463 |
| 41 | 5Hex | 828.29 | 11.508 | 4.91 | 3.627 |
| 42 | 5Hex1Pent | 960.33 | 13.873 | 4.53 | 4.372 |
| 43 | 5Hex1Pent | 960.33 | 15.799 | 0.25 | 4.979 |
| 44 | 6Hex | 990.34 | 13.474 | 2.96 | 4.246 |
| 45 | 6Hex1Pent | 1122.38 | 15.097 | 2.23 | 4.758 |
| 46 | 7Hex | 1152.39 | 14.813 | 0.26 | 4.668 |
| 47 | 7Hex | 1152.39 | 16.921 | 0.23 | 5.333 |

Baobob powder refers to a natural product that is high in fiber. Baobob powder can be derived from the fruit of trees in the genus Adansonia. The produced oligosaccharides possessed various structural features similar to those in the parent polysaccharide. The glucose composition being 35.03%, followed by 17.84% xylose, 12.4% galactose and 12.1% galacturonic acid (Table 3B). With the glycosidic linkage composition being 15.49% terminal xylose, followed by 2,3,4-xylose at 13.06%, 4-glucose at 9.03% 4-xylose at 5.82%, and 2,3,5 arabinose at 4.67% (Table 4B). 64 oligosaccharides were observed in the pool that ranged from a hexose and 2 pentose to 6 hexoses in length. The most abundant structures represent 3Hex, 2.626 min and 14.719 min; 2Hex1Pent, 7.217 min and 10.287 min; Hex1Pent1Deoxy, 13.753 min; 2Hex2Pent, 14.538 min; 4Hex, 25.019; 5Hex, 29.298 min. The full list of oligosaccharide peaks and abundances are found in Table 35. The oligosaccharide pool can be further distinguished by 1H-13C 2D-NMR (HSQC). Prominent peaks include those described in Table 5B.

TABLE 35
Oligosaccharides generated from the COG depolymerization of
Baobob Powder. Hex refers to hexose sugars, Pent refers to
pentose sugars, HexA refers to hexuronic acid sugars, and Deoxyhex
refers to deoxyhexose sugar. Where Hexose refers to either
galactose or glucose, pentose refers to either arabinose or
xylose and Deoxyhex refers to either rhamnose or fucose.

| | | | RT | Oligo | Retention |
|---|---|---|---|---|---|
| Compound | Identity | Mass | (min) | Wt % | Factor |
| 1 | 1Hex1Pent1Deoxy | 458.18 | 13.753 | 9.1 | 9.369 |
| 2 | 1Hex2Pent | 444.17 | 7.755 | 0.62 | 5.283 |
| 3 | 2Hex1Pent | 474.17 | 2.192 | 0.36 | 1.493 |
| 4 | 2Hex1Pent | 474.17 | 3.018 | 2.38 | 2.056 |
| 5 | 2Hex1Pent | 474.17 | 3.925 | 1.0 | 2.674 |
| 6 | 2Hex1Pent | 474.17 | 6.7 | 2.53 | 4.564 |
| 7 | 2Hex1Pent | 474.17 | 7.217 | 3.35 | 4.916 |
| 8 | 2Hex1Pent | 474.17 | 9.613 | 0.74 | 6.548 |
| 9 | 2Hex1Pent | 474.17 | 10.081 | 0.93 | 6.867 |
| 10 | 2Hex1Pent | 474.17 | 10.287 | 6.07 | 7.007 |
| 11 | 2Hex1Pent | 474.17 | 11.209 | 1.16 | 7.636 |
| 12 | 2Hex2Pent | 606.22 | 14.538 | 4.69 | 9.903 |
| 13 | 3Hex | 504.18 | 1.468 | 0.95 | 1 |
| 14 | 3Hex | 504.18 | 2.139 | 0.35 | 1.457 |
| 15 | 3Hex | 504.18 | 2.626 | 3.54 | 1.789 |
| 16 | 3Hex | 504.19 | 2.861 | 0.49 | 1.949 |
| 17 | 3Hex | 504.18 | 3.918 | 2.44 | 2.669 |
| 18 | 3Hex | 504.18 | 7.895 | 1.28 | 5.378 |
| 19 | 3Hex | 504.18 | 9.287 | 2.82 | 6.326 |
| 20 | 3Hex | 504.18 | 13.517 | 0.71 | 9.208 |
| 21 | 3Hex | 504.18 | 14.719 | 5.66 | 10.027 |
| 22 | 3Hex1Pent | 636.23 | 7.914 | 1.48 | 5.391 |
| 23 | 3Hex1Pent | 636.23 | 12.155 | 0.72 | 8.28 |
| 24 | 3Hex1Pent | 636.23 | 13.286 | 0.99 | 9.05 |
| 25 | 3Hex1Pent | 636.23 | 18.281 | 0.96 | 12.453 |
| 26 | 3Hex1Pent | 636.23 | 19.503 | 0.43 | 13.285 |
| 27 | 3Hex1Pent | 636.23 | 19.974 | 1.33 | 13.606 |
| 28 | 3Hex1Pent | 636.23 | 21.372 | 1.5 | 14.559 |
| 29 | 3Hex2Pent | 768.27 | 16.654 | 0.96 | 11.345 |
| 30 | 3Hex2Pent | 768.27 | 17.425 | 2.14 | 11.87 |
| 31 | 3Hex2Pent | 768.27 | 22.775 | 1.23 | 15.514 |
| 32 | 3Hex2Pent | 768.27 | 23.886 | 1.76 | 16.271 |
| 33 | 3Hex2Pent | 768.27 | 24.76 | 1.55 | 16.866 |
| 34 | 3Hex3Pent | 900.31 | 26.171 | 0.64 | 17.828 |
| 35 | 3Pent | 414.15 | 8.896 | 1.71 | 6.06 |
| 36 | 4Hex | 666.24 | 3.813 | 0.34 | 2.597 |
| 37 | 4Hex | 666.24 | 6.154 | 0.65 | 4.192 |
| 38 | 4Hex | 666.24 | 7.092 | 1.63 | 4.831 |
| 39 | 4Hex | 666.24 | 8.621 | 0.52 | 5.873 |
| 40 | 4Hex | 666.24 | 9.904 | 1.71 | 6.747 |
| 41 | 4Hex | 666.24 | 11.461 | 0.84 | 7.807 |
| 42 | 4Hex | 666.24 | 12.074 | 1.44 | 8.225 |
| 43 | 4Hex | 666.24 | 19.096 | 1.85 | 13.008 |
| 44 | 4Hex | 666.24 | 25.019 | 4.35 | 17.043 |
| 45 | 4Hex1Pent | 798.28 | 11.097 | 1.11 | 7.559 |
| 46 | 4Hex1Pent | 798.28 | 29.048 | 0.45 | 19.787 |
| 47 | 4Hex2Pent | 930.32 | 24.983 | 1.12 | 17.018 |
| 48 | 4Hex2Pent | 930.32 | 29.226 | 0.49 | 19.909 |
| 49 | 4Pent | 546.2 | 17.389 | 1.2 | 11.845 |
| 50 | 5Hex | 828.29 | 9.75 | 0.49 | 6.642 |
| 51 | 5Hex | 828.29 | 10.592 | 1.2 | 7.215 |
| 52 | 5Hex | 828.29 | 13.065 | 0.97 | 8.9 |
| 53 | 5Hex | 828.29 | 19.612 | 0.95 | 13.36 |
| 54 | 5Hex | 828.29 | 20.778 | 1.15 | 14.154 |
| 55 | 5Hex | 828.29 | 26.34 | 0.64 | 17.943 |
| 56 | 5Hex | 828.29 | 27.136 | 0.89 | 18.485 |
| 57 | 5Hex | 828.29 | 29.298 | 3.67 | 19.958 |
| 58 | 5Hex1Pent | 960.33 | 12.982 | 0.62 | 8.843 |
| 59 | 5Pent | 678.24 | 23.767 | 0.63 | 16.19 |
| 60 | 6Hex | 990.34 | 12.6 | 0.61 | 8.583 |
| 61 | 6Hex | 990.34 | 26.762 | 0.63 | 18.23 |
| 62 | 6Hex | 990.34 | 27.016 | 0.28 | 18.403 |
| 63 | 6Hex | 990.34 | 27.338 | 0.48 | 18.623 |
| 64 | 6Hex | 990.34 | 29.982 | 0.49 | 20.424 |

Lupin glactan refers to purified polysaccaride extract from the lupin bean of the lupin tree in the genus Lupinus. As used herein, lupin galactan is a polysaccharide with a glycosidic linkage composition comprising a β-1,4 galactan backbone. The produced oligosaccharides possessed various structural features similar to those in the parent polysaccharide. Lupin Galactan oligosaccharides have a composition wherein about 77% of the mass comprises galactose, about 9% of the mass comprises arabinose, and about 3% of the mass comprises rhamnose, as measured by hydrolytic monosaccharide compositional analysis. With the glycosidic linkage composition comprises about 61% 4-linked galactose, about 21% terminal galactose, about 5% 5-linked galactose, and about 2% terminal arabinose. The most abundant structures represent 3Hex, 3.098 min; 4Hex, 7.754 min; 3Hex1Pent, 8.574 min; Hex, 11.246 min; 4Hex1Pent, 11.786; 5HexPent, 13.697 min. The full list of oligosaccharide peaks and abundances are found in Table 36. The oligosaccharide pool can be further distinguished by its 1H-13C 2D-NMR (HSQC) fingerprint (FIG. 13B). Prominent peaks include those described in Table 5B.

TABLE 36
Oligosaccharides generated from the COG depolymerization of Lupin
Galactan. Hex refers to hexose sugars, Pent refers to pentose
sugars, HexA refers to hexuronic acid sugars, and Deoxyhex refers
to deoxyhexose sugars. Where hexoses are galactose, pentoses
are arabinose and hexuronic acids are Galacturonic acid.

| | | | RT | Oligo | Retention |
|---|---|---|---|---|---|
| Compound | Identity | Mass | (min) | Wt % | Factor |
| 1 | 2Hex1HexA | 518.17 | 4.698 | 3.82 | 1.536 |
| 2 | 2Hex1Pent | 474.18 | 3.058 | 0.80 | 1 |
| 3 | 2Hex1Pent | 474.18 | 3.57 | 9.46 | 1.167 |
| 4 | 2Hex1Pent | 474.17 | 3.886 | 0.86 | 1.271 |
| 5 | 3Hex | 504.19 | 3.098 | 10.67 | 1.013 |
| 6 | 3Hex | 504.19 | 4.06 | 1.10 | 1.328 |
| 7 | 3Hex1HexA | 680.22 | 9.814 | 5.15 | 3.209 |
| 8 | 3Hex1Pent | 636.23 | 7.045 | 1.01 | 2.304 |
| 9 | 3Hex1Pent | 636.23 | 7.508 | 0.82 | 2.455 |
| 10 | 3Hex1Pent | 636.23 | 7.831 | 0.60 | 2.561 |
| 11 | 3Hex1Pent | 636.23 | 8.574 | 12.31 | 2.804 |
| 12 | 4Hex | 666.24 | 7.754 | 13.60 | 2.536 |
| 13 | 4Hex | 666.24 | 8.957 | 0.93 | 2.929 |
| 14 | 4Hex1HexA | 842.27 | 13.063 | 3.37 | 4.272 |
| 15 | 4Hex1Pent | 798.28 | 10.559 | 0.47 | 3.453 |
| 16 | 4Hex1Pent | 798.28 | 10.862 | 0.83 | 3.552 |
| 17 | 4Hex1Pent | 798.28 | 11.786 | 9.98 | 3.854 |
| 18 | 5Hex | 828.29 | 11.246 | 9.46 | 3.678 |
| 19 | 5Hex | 828.29 | 11.945 | 0.54 | 3.906 |
| 20 | 5Hex1HexA | 1004.32 | 14.971 | 2.22 | 4.896 |
| 21 | 5Hex1Pent | 960.33 | 13.016 | 0.53 | 4.256 |
| 22 | 5Hex1Pent | 960.33 | 13.697 | 5.16 | 4.479 |
| 23 | 6Hex | 990.34 | 13.315 | 2.17 | 4.354 |
| 24 | 6Hex1HexA | 1166.38 | 16.133 | 0.91 | 5.276 |
| 25 | 6Hex1Pent | 1122.38 | 14.908 | 1.83 | 4.875 |
| 26 | 7Hex | 1152.4 | 14.632 | 0.86 | 4.785 |
| 27 | 7Hex1Pent | 1284.44 | 16.044 | 0.53 | 5.247 |

Destarched pea fiber refers to a natural product polysaccharide with a α-1,5 arabinose backbone with either α-1,2 or α-1,3 arabinose decorations with depleted starch levels by common methods. Destarched pea fiber can be derived from peas or processed fractions thereof. The produced oligosaccharides possessed various structural features similar to those in the parent polysaccharide. The arabinose composition being 76.85%, followed by 9.78% glucose and 5.47% galactose (Table 3B). With the glycosidic linkage composition being 47.11% terminal arabinose, followed by 21.21% 5-arabinose, 6.36% 2,5-arabinose, 5.13% 3,5, arabinose, and 3.71% 4-glucose (Table 4B). 53 oligosaccharides were observed in the pool that ranged from 2 hexose and a pentose to 9 hexoses in length. The most abundant structures represent 3Hex, 5.362 min, 3Pent, 6.573 min and 11.502 min; 4Hex 10.938 min; 4Pent, 11.497 min; 5Hex 13.869 min; 6Hex, 15.605 min. The full list of oligosaccharide peaks and abundances are found in Table 37. The oligosaccharide pool can be further distinguished by 1H-13C 2D-NMR (HSQC). Prominent peaks include those described in Table 5B.

TABLE 37
Oligosaccharides generated from the COG depolymerization
of Destarched pea fiber. Hex refers to hexose sugars,
Pent refers to pentose sugars, HexA refers to hexuronic
acid sugars, and Deoxyhex refers to deoxyhexose sugar.

| | | | RT | Oligo | Retention |
|---|---|---|---|---|---|
| Compound | Identity | Mass | (min) | Wt % | Factor |
| 1 | 2Hex1Pent | 474.17 | 4.036 | 0.69 | 1.16 |
| 2 | 2Hex1Pent | 474.17 | 5.323 | 1.46 | 1.53 |
| 3 | 2Hex1Pent | 474.18 | 11.305 | 0.61 | 3.25 |
| 4 | 3Hex | 504.18 | 3.483 | 0.88 | 1 |
| 5 | 3Hex | 504.18 | 5.362 | 5.51 | 1.54 |
| 6 | 3Hex | 504.18 | 15.448 | 1.93 | 4.44 |
| 7 | 3Hex | 504.19 | 15.767 | 0.59 | 4.53 |
| 8 | 3Hex1Pent | 636.23 | 9.205 | 0.5 | 2.64 |
| 9 | 3Hex1Pent | 636.22 | 10.992 | 1.77 | 3.16 |
| 10 | 3Pent | 414.15 | 4.026 | 2.44 | 1.16 |
| 11 | 3Pent | 414.15 | 5.603 | 1.13 | 1.61 |
| 12 | 3Pent | 414.15 | 6.573 | 8.31 | 1.89 |
| 13 | 3Pent | 414.15 | 7.332 | 1.48 | 2.11 |
| 14 | 3Pent | 414.15 | 10.03 | 1.11 | 2.88 |
| 15 | 3Pent | 414.15 | 10.802 | 2.03 | 3.1 |
| 16 | 3Pent | 414.15 | 11.502 | 4.27 | 3.3 |
| 17 | 3Pent | 414.15 | 17.973 | 1.17 | 5.16 |
| 18 | 4Hex | 666.24 | 8.529 | 0.68 | 2.45 |
| 19 | 4Hex | 666.24 | 10.938 | 6.48 | 3.14 |
| 20 | 4Hex | 666.24 | 19.58 | 0.53 | 5.62 |
| 21 | 4Hex | 666.24 | 25.163 | 1.64 | 7.22 |
| 22 | 4Hex1Pent | 798.28 | 12.023 | 0.56 | 3.45 |
| 23 | 4Hex1Pent | 798.28 | 13.963 | 1.87 | 4.01 |
| 24 | 4Pent | 546.19 | 6.57 | 0.63 | 1.89 |
| 25 | 4Pent | 546.2 | 10.783 | 1.23 | 3.1 |
| 26 | 4Pent | 546.2 | 10.825 | 0.99 | 3.11 |
| 27 | 4Pent | 546.19 | 11.155 | 0.92 | 3.2 |
| 28 | 4Pent | 546.19 | 11.497 | 4.39 | 3.3 |
| 29 | 4Pent | 546.19 | 13.818 | 1.5 | 3.97 |
| 30 | 4Pent | 546.19 | 16.241 | 0.89 | 4.66 |
| 31 | 4Pent | 546.19 | 17.957 | 1.58 | 5.16 |
| 32 | 5Hex | 828.29 | 11.567 | 0.5 | 3.32 |
| 33 | 5Hex | 828.29 | 13.869 | 7.43 | 3.98 |
| 34 | 5Hex | 828.29 | 29.199 | 1.48 | 8.38 |
| 35 | 5Hex1Pent | 960.33 | 15.681 | 1.24 | 4.5 |
| 36 | 5Pent | 678.24 | 15.754 | 3.37 | 4.52 |
| 37 | 5Pent | 678.24 | 16.235 | 1.19 | 4.66 |
| 38 | 5Pent | 678.24 | 17.874 | 1.11 | 5.13 |
| 39 | 5Pent | 678.24 | 19.37 | 0.42 | 5.56 |
| 40 | 5Pent | 678.24 | 20.327 | 0.89 | 5.84 |
| 41 | 6Hex | 990.34 | 15.605 | 5.63 | 4.48 |
| 42 | 6Hex1Pent | 1122.38 | 16.842 | 0.74 | 4.84 |
| 43 | 6Pent | 810.32 | 6.567 | 0.47 | 1.89 |
| 44 | 6Pent | 810.28 | 17.192 | 0.6 | 4.94 |
| 45 | 6Pent | 810.28 | 18.685 | 1 | 5.36 |
| 46 | 6Pent | 810.28 | 19.018 | 1.77 | 5.46 |
| 47 | 6Pent | 810.28 | 19.725 | 1.79 | 5.66 |
| 48 | 7Hex | 1152.39 | 16.777 | 3.51 | 4.82 |
| 49 | 7Pent | 942.32 | 20.319 | 1.36 | 5.83 |
| 50 | 7Pent | 942.32 | 22.471 | 1.29 | 6.45 |
| 51 | 8Hex | 1314.45 | 17.774 | 2 | 5.1 |
| 52 | 8Pent | 1074.36 | 23.36 | 1.53 | 6.71 |
| 53 | 9Hex | 1476.5 | 19.142 | 0.91 | 5.5 |

### Example 11

### (Comparison of COG and FITDOG Products)

Oligosaccharides produced by COG were expected to differ from those produced by a similar method referred to as FITDOG in PCT Application No., PCT/US2018/038350, published as WO/2018/936917. Oligosaccharides were found to have some homogeneity between pools, however, substantial differences were also encountered. COG was applied to galactomannan, arabinoxylan, xyloglucan, glucomannan, lichenan, mannan, galactan, β-glucan, curdlan, and xylan. The results were analyzed via mass spectrometry and the peaks corresponding to oligosaccharides were compared with those described in PCT Application No. PCT/US2020/035748.

COG Oligosaccharide production: Galactomannan, arabinoxylan, xyloglucan, glucomannan, lichenan, mannan, galactan, β-glucan, curdlan, and xylan (550 mg) were dissolved in 20 ml of HPLC grade water in a capped reaction vessel and placed in a shaker-incubator for 20 min at 55° C. and 85 RPM. The pH of the solution was adjusted to 5.2. Hydrogen peroxide (5 ml) and iron (II) sulfate (2.75 mg in 50 μL water) were added to the reaction mixture and mixed thoroughly, except for curdlan where copper (II) sulfate was used. The reaction in the capped reaction vessel proceeded in the shaker-incubator at 55° C. and 65 RPM for two hours. The capped reaction cooled to 12° C. in a −20° C. freezer. Ammonium hydroxide (1 ml of 28% v/v to pH 10.2) was used to adjust pH and sample was reacted at 45° C. in a shaker-incubator for 1 hour at 20 RPM, the cap was left loose to allow oxygen, ammonia, and carbon dioxide gases to be released. The sample is then frozen and lyophilized, then stored at −80° C. The freeze-dried oligosaccharide mixture was rehydrated with the minimum amount of water required to allow for a free-flowing solution. This solution was then loaded onto a column containing 15 mL mixed bed ion exchange resin per gram (dry weight) of crude material, and the runoff was collected in a plastic freezer bag. Once the material was loaded onto the column, the column was then rinsed with 3 bed volumes of water. Finally, the runoff was sealed and frozen in the bag, then carefully shattered and subjected to lyophilization.

Data analysis of COG Products: Oligosaccharide analysis was performed in the manner of Amicucci, M. J., Nandita, E., et al. (2020). Nature Communications 11(1): 1-12. Oligosaccharide peak volumes were generated from Agilent Mass Hunter Qualitative Analysis B.10 by using their “find by molecular feature” function.

FITDOG Oligosaccharide production: A solution was prepared containing 95% (v/v) sodium acetate buffer adjusted to pH 5 with glacial acetic acid, 5% (v/v) hydrogen peroxide (30% w/w), and 65 μM of the metal complex under investigation. This mixture was vortexed and was added to Galactomannan, arabinoxylan, xyloglucan, glucomannan, lichenan, mannan, galactatan, β-glucan, curdlan, and xylan to make a final solution of 1 mg/ml. The reaction was incubated at 100° C. for 60 minutes. After reacting, half of the reaction volume of cold 2 M NaOH was added and vortexed before adding 0.6% of the initial reaction volume of glacial acetic acid to neutralize.

Oligosaccharides were isolated using nonporous graphitized carbon cartridges (GCC-SPE). Cartridges were washed with 80% acetonitrile in 0.1% (v/v) trifluoroacetic acid (TFA) and nano-pure water. The oligosaccharides were loaded and washed with 5 column volumes of nano-pure water. The oligosaccharides were eluted with 40% acetonitrile with 0.05% (v/v) TFA.

Data analysis of FITDOG Products: Oligosaccharides from FITDOG were manually annotated from their parent mass as obtained through the Agilent MassHunter Qualitative Analysis. For this example, data were obtained directly from PCT Application No. PCT/US2020/035748.

Several trends were noticed when comparing the COG and FITDOG samples. The entirety of the data is presented in Table 38; unique oligosaccharides are marked for each process, while similar oligosaccharides can be deduced from the differences between Table 38 and Tables 7-10 and 12-17. Furthermore, the mass of the compounds in Table 38 can be referenced with Tables 7-10 and 12-17 for their compositional identities. For galactomannan COG produced many compounds comprising of 3-5 hexoses and a single pentose; whereas the FITDOG process included two differentiated 7Hex isomers. For arabinoxylan, COG produced several small DP3 and DP4 pentose oligosaccharides that were unique, while FITDOG produced several other isomers ranging from DP3-DP11 with a number of high DP isomers that were not produced by COG. For xyloglucan, FITDOG tended to produce more isomers of large DP, while COG produced shorter oligosaccharides. For glucomannan, COG produced a number of isomers that contained hexoses and a single pentose unit that were not produced by FITDOG. For galactan, COG produced a number of unique isomers that contained hexoses and a single pentose unit, while FITDOG produced several larger, DP8 and DP9 oligosaccharides that were not found in COG. For β-glucan, FITDOG produced more isomers of DP6 and DP7. For lichenan, COG produced a number of unique isomers that contained hexoses and a single pentose unit, while FITDOG produced many unique DP3-DP10 oligosaccharides that were not found in COG. For mannan, COG produced a number of unique isomers that contained hexoses and a single pentose unit, while FITDOG produced many unique DP4-DP9 oligosaccharides that were not found in COG. For xylan, the FITDOG process produced more unique oligosaccharides with methylated glucuronic acid residues. For curdlan, COG produced unique oligosaccharides with unique isomers that contained hexoses and a single pentose unit as well as one unique DP3 oligosaccharide.

Herein disclosed are synthetic oligosaccharides, including pools of oligosaccharides, which are produced by the COG process, comprising at least 1, 5, 10, 15, 20, 25, 30, 35, 40, 45, 50 or all of the oligosaccharides referenced in Table 38 as being unique to the COG process. Further disclosed herein are synthetic oligosaccharides, including pools of oligosaccharides, which are produced by the COG process, but wherein oligosaccharides referenced in Table 38 as being unique to the FITDOG process are not present at detectable levels in the COG produced oligosaccharides,

TABLE 38

| | | | Unique | | | | | Unique | |
|---|---|---|---|---|---|---|---|---|---|
| | RT | Unique | to | Pool | | RT | Unique | to | Pool |
| Mass | (min) | to COG | FITDOG | Identity | Mass | (min) | to COG | FITDOG | Identity |
| 636.230 | 6.404 | x | | GalMan | 414.150 | 11.556 | x | | ArabXyl |
| 636.230 | 6.968 | x | | GalMan | 414.150 | 21.696 | x | | ArabXyl |
| 636.230 | 8.198 | x | | GalMan | 678.240 | 25.947 | x | | ArabXyl |
| 636.230 | 8.566 | x | | GalMan | 414.140 | 2.000 | | x | ArabXyl |
| 768.270 | 10.827 | x | | GalMan | 546.180 | 11.200 | | x | ArabXyl |
| 768.270 | 12.342 | x | | GalMan | 546.180 | 11.580 | | x | ArabXyl |
| 768.270 | 13.372 | x | | GalMan | 546.180 | 12.160 | | x | ArabXyl |
| 812.260 | 10.495 | x | | GalMan | 678.220 | 15.820 | | x | ArabXyl |
| 812.260 | 14.627 | x | | GalMan | 678.220 | 17.150 | | x | ArabXyl |
| 842.270 | 9.515 | x | | GalMan | 678.220 | 18.790 | | x | ArabXyl |
| 842.270 | 10.301 | x | | GalMan | 810.260 | 14.030 | | x | ArabXyl |
| 842.270 | 11.020 | x | | GalMan | 942.310 | 16.610 | | x | ArabXyl |
| 842.270 | 11.725 | x | | GalMan | 942.310 | 18.290 | | x | ArabXyl |
| 842.270 | 12.330 | x | | GalMan | 942.310 | 22.540 | | x | ArabXyl |
| 842.270 | 14.866 | x | | GalMan | 1074.350 | 26.330 | | x | ArabXyl |
| 842.270 | 16.706 | x | | GalMan | 1074.350 | 26.830 | | x | ArabXyl |
| 798.280 | 9.614 | x | | GalMan | 1074.350 | 27.100 | | x | ArabXyl |
| 798.280 | 10.996 | x | | GalMan | 1074.350 | 27.490 | | x | ArabXyl |
| 798.280 | 11.525 | x | | GalMan | 1074.350 | 28.150 | | x | ArabXyl |
| 798.280 | 14.191 | x | | GalMan | 1206.390 | 26.370 | | x | ArabXyl |
| 1152.390 | 14.302 | x | | GalMan | 1206.390 | 27.100 | | x | ArabXyl |
| 1152.380 | 13.570 | | x | GalMan | 1338.430 | 27.100 | | x | ArabXyl |
| 1152.380 | 13.720 | | x | GalMan | 1470.480 | 28.630 | | x | ArabXyl |
| 606.220 | 14.055 | x | | XylGlc | 474.170 | 2.263 | x | | GlcMan |
| 768.270 | 22.600 | x | | XylGlc | 636.230 | 6.413 | x | | GlcMan |
| 930.320 | 24.564 | x | | XylGlc | 474.170 | 9.187 | x | | GlcMan |
| 1224.420 | 30.009 | x | | XylGlc | 798.280 | 9.604 | x | | GlcMan |
| 930.320 | 30.836 | x | | XylGlc | 474.170 | 10.653 | x | | GlcMan |
| 1224.420 | 31.349 | x | | XylGlc | 636.230 | 12.074 | x | | GlcMan |
| 1062.360 | 31.490 | x | | XylGlc | 474.170 | 12.713 | x | | GlcMan |
| 1092.360 | 18.980 | | x | XylGlc | 636.230 | 13.068 | x | | GlcMan |
| 1092.360 | 19.940 | | x | XylGlc | 798.280 | 15.280 | x | | GlcMan |
| 1092.360 | 23.140 | | x | XylGlc | 798.280 | 17.219 | x | | GlcMan |
| 1092.360 | 23.740 | | x | XylGlc | 798.280 | 18.793 | x | | GlcMan |
| 1092.360 | 24.780 | | x | XylGlc | 636.230 | 18.947 | x | | GlcMan |
| 1092.360 | 25.440 | | x | XylGlc | 636.230 | 19.573 | x | | GlcMan |
| 1224.400 | 21.270 | | x | XylGlc | 636.230 | 20.258 | x | | GlcMan |
| 1224.400 | 24.960 | | x | XylGlc | 798.280 | 20.328 | x | | GlcMan |
| 1224.400 | 25.810 | | x | XylGlc | 798.280 | 21.012 | x | | GlcMan |
| 1254.410 | 22.740 | | x | XylGlc | 798.280 | 21.923 | x | | GlcMan |
| 1386.450 | 26.470 | | x | XylGlc | 798.280 | 22.714 | x | | GlcMan |
| 1386.450 | 26.730 | | x | XylGlc | 798.280 | 26.723 | x | | GlcMan |
| 1386.450 | 27.330 | | x | XylGlc | 798.280 | 27.138 | x | | GlcMan |
| 474.180 | 2.658 | x | | Gal | 798.280 | 27.347 | x | | GlcMan |
| 474.170 | 3.038 | x | | Gal | 798.280 | 27.565 | x | | GlcMan |
| 636.230 | 6.014 | x | | Gal | 990.330 | 13.350 | | x | β-Glc |
| 636.230 | 7.292 | x | | Gal | 990.330 | 22.790 | | x | β-Glc |
| 680.220 | 8.937 | x | | Gal | 990.330 | 25.100 | | x | β-Glc |
| 798.280 | 9.508 | x | | Gal | 1152.380 | 26.050 | | x | β-Glc |
| 798.280 | 10.112 | x | | Gal | 1152.380 | 27.230 | | x | β-Glc |
| 842.270 | 11.857 | x | | Gal | 1152.380 | 27.470 | | x | β-Glc |
| 812.260 | 13.126 | x | | Gal | 1152.380 | 28.610 | | x | β-Glc |
| 1004.320 | 13.573 | x | | Gal | 312.120 | 1.583 | x | | Mann |
| 1166.370 | 14.673 | x | | Gal | 474.180 | 6.624 | x | | Mann |
| 974.310 | 14.753 | x | | Gal | 474.180 | 6.954 | x | | Mann |
| 1314.430 | 14.060 | | x | Gal | 444.170 | 7.463 | x | | Mann |
| 1476.490 | 14.750 | | x | Gal | 444.160 | 7.984 | x | | Mann |
| 504.190 | 1.488 | x | | Lich | 474.180 | 9.655 | x | | Mann |
| 474.180 | 2.211 | x | | Lich | 606.220 | 10.576 | x | | Mann |
| 342.130 | 2.925 | x | | Lich | 636.230 | 11.423 | x | | Mann |
| 444.170 | 2.988 | x | | Lich | 474.180 | 12.409 | x | | Mann |
| 666.240 | 4.016 | x | | Lich | 636.230 | 12.727 | x | | Mann |
| 666.240 | 4.726 | x | | Lich | 606.220 | 13.770 | x | | Mann |
| 650.240 | 5.754 | x | | Lich | 606.220 | 16.102 | x | | Mann |
| 680.220 | 5.977 | x | | Lich | 768.270 | 16.706 | x | | Mann |
| 636.230 | 6.275 | x | | Lich | 636.230 | 17.531 | x | | Mann |
| 636.230 | 6.830 | x | | Lich | 606.220 | 18.235 | x | | Mann |
| 636.230 | 8.396 | x | | Lich | 930.320 | 18.491 | x | | Mann |
| 606.220 | 8.398 | x | | Lich | 606.220 | 19.122 | x | | Mann |
| 798.280 | 9.459 | x | | Lich | 636.230 | 19.328 | x | | Mann |
| 842.270 | 9.907 | x | | Lich | 798.280 | 19.731 | x | | Mann |
| 812.260 | 10.220 | x | | Lich | 636.230 | 20.683 | x | | Mann |
| 842.270 | 10.504 | x | | Lich | 798.290 | 21.950 | x | | Mann |
| 768.270 | 10.680 | x | | Lich | 768.270 | 22.303 | x | | Mann |
| 960.340 | 12.139 | x | | Lich | 930.320 | 22.608 | x | | Mann |
| 1004.330 | 12.747 | x | | Lich | 768.270 | 23.412 | x | | Mann |
| 1004.320 | 13.358 | x | | Lich | 1092.380 | 24.100 | x | | Mann |
| 974.310 | 13.623 | x | | Lich | 930.320 | 24.422 | x | | Mann |
| 1122.390 | 14.439 | x | | Lich | 930.320 | 24.785 | x | | Mann |
| 1166.380 | 15.181 | x | | Lich | 768.270 | 24.786 | x | | Mann |
| 666.240 | 16.429 | x | | Lich | 1092.380 | 25.241 | x | | Mann |
| 504.170 | 11.580 | | x | Lich | 768.270 | 25.534 | x | | Mann |
| 504.170 | 13.730 | | x | Lich | 798.280 | 25.871 | x | | Mann |
| 504.170 | 15.240 | | x | Lich | 960.340 | 26.217 | x | | Mann |
| 828.270 | 26.590 | | x | Lich | 960.330 | 26.435 | x | | Mann |
| 1152.380 | 9.590 | | x | Lich | 798.280 | 26.802 | x | | Mann |
| 1152.380 | 9.990 | | x | Lich | 680.220 | 27.032 | x | | Mann |
| 1152.380 | 10.500 | | x | Lich | 930.320 | 27.594 | x | | Mann |
| 1152.380 | 11.190 | | x | Lich | 1092.380 | 27.782 | x | | Mann |
| 1152.380 | 11.970 | | x | Lich | 812.260 | 28.218 | x | | Mann |
| 1152.380 | 15.650 | | x | Lich | 1092.380 | 28.330 | x | | Mann |
| 1152.380 | 17.240 | | x | Lich | 930.320 | 28.643 | x | | Mann |
| 1152.380 | 17.580 | | x | Lich | 1092.380 | 29.139 | x | | Mann |
| 1314.430 | 11.160 | | x | Lich | 812.260 | 29.164 | x | | Mann |
| 1314.430 | 18.170 | | x | Lich | 680.220 | 29.171 | x | | Mann |
| 1314.430 | 20.180 | | x | Lich | 812.260 | 30.672 | x | | Mann |
| 1476.490 | 20.810 | | x | Lich | 930.320 | 30.759 | x | | Mann |
| 1638.540 | 23.510 | | x | Lich | 666.220 | 3.510 | | x | Mann |
| 1641.560 | 23.570 | | x | Lich | 828.270 | 9.150 | | x | Mann |
| 1314.430 | 18.170 | | x | Lich | 990.330 | 11.930 | | x | Mann |
| 1314.430 | 20.180 | | x | Lich | 1152.380 | 13.100 | | x | Mann |
| 1476.490 | 20.810 | | x | Lich | 1314.430 | 14.060 | | x | Mann |
| 1641.560 | 23.570 | | x | Lich | 1476.490 | 14.750 | | x | Mann |
| 282.110 | 6.692 | x | | Xyl | 342.130 | 1.456 | x | | Curd |
| 1092.420 | 8.109 | x | | Xyl | 312.120 | 1.663 | x | | Curd |
| 678.240 | 8.432 | x | | Xyl | 504.180 | 7.423 | x | | Curd |
| 1122.350 | 18.442 | x | | Xyl | 474.170 | 12.672 | x | | Curd |
| 736.240 | 26.435 | x | | Xyl | 474.170 | 13.644 | x | | Curd |
| 942.320 | 28.974 | x | | Xyl | 636.230 | 24.761 | x | | Curd |
| 604.190 | 14.000 | | x | Xyl | | | | | |
| 604.190 | 14.270 | | x | Xyl | | | | | |
| 810.260 | 21.920 | | x | Xyl | | | | | |
| 868.270 | 20.930 | | x | Xyl | | | | | |
| 868.270 | 22.820 | | x | Xyl | | | | | |
| 868.270 | 23.130 | | x | Xyl | | | | | |
| 868.270 | 23.460 | | x | Xyl | | | | | |
| 868.270 | 24.870 | | x | Xyl | | | | | |
| 1000.32 | 24.51 | | x | Xyl | | | | | |
| 1000.32 | 26.88 | | x | Xyl | | | | | |
| 1000.32 | 27.34 | | x | Xyl | | | | | |
| 1000.32 | 27.52 | | x | Xyl | | | | | |
| 1000.32 | 28.51 | | x | Xyl | | | | | |
| 1000.32 | 29.39 | | x | Xyl | | | | | |
| 1074.35 | 28.15 | | x | Xyl | | | | | |
| 1132.36 | 27.61 | | x | Xyl | | | | | |
| 1132.36 | 28.47 | | x | Xyl | | | | | |
| 1132.36 | 29.69 | | x | Xyl | | | | | |

GalMan = Galactomannan,
ArabXyl = Arabinoxylan,
XylGlc = Xyloglucan,
GlcMan = Glucomannan,
Lich = Lichenan,
Man = Mannan,
Gal = Galactan,
β-Glc = Beta-Glucan,
Curd = Curdlan,
Xyl = Xylan

### Example 12

### (Effect of Oligosaccharides on Single Strain Growth)

To determine the oligosaccharide that best suits specific microbial strain growth, selected microbes were grown in the presence of oligosaccharides a sole carbon source and assayed for growth and metabolic output. Selected strains of Lactobacillus crispatus, Lactobacillus rhamnosus, Bifidobacteria longum subsp. infantis, Bifidobacteria longum subsp. longum, Clostridium butyricum, Bifidobacterium bifidum and Bifidobacteria pseudocatenulatum from the American Type Culture Collection and isolated from human specimens were employed as the microbes in this example.

Selected strains were incubated in minimal media containing the following oligos in a final concentration of 2% w/v: CLX101, CLX102, CLX103, CLX105, CLX107, CLX108, CLX109, CLX110, CLX111, CLX112, CLX113, CLX114, CLX116, CLX117, CLX119, CLX115, CLX121, CLX122, CLX123, CLX124, CLX125, CLX126, CLX127, CLX128, CLX129, CLX130 and CLX131. Growth was monitored by measuring OD (600 n) using microtiter plate-reader (Epoch 2, BioTek). FOS, GOS and inulin were included as controls. Specifically, fresh cultures were generated by transferring a colony (per replicate) into fresh liquid media and incubating in an anaerobic chamber (Anaerobic Chamber Vinyl Type B), using a mix of gas (carbon dioxide 5%, hydrogen 5%, nitrogen balance). De Man, Rogosa 15 and Sharpe media (MRS) was used to propagate the following organisms: Lactobacillus crispatus, Lactobacillus rhamnosus, Bifidobacterium bifidum, Bifidobacterium longum subsp. infantis, Bifidobacterium longum subsp. longum Bifidobacterium pseudocatenulatum and Clostridium butyricum. Cells were harvested at late exponential phase and an experimental inoculum was prepared by washing once with phosphate buffered saline (PBS). This was done by centrifuging the culture at 8000 g for 5 min, discarding the supernatant, resuspending in PBS and precipitating the cells once more before resuspending cells again fresh basal (no carbohydrate) media.

Differential growth of tested strains was observed due to their unique utilization of each oligo when supplemented as the sole source of carbon. The number of strains that each fraction supported growth on varies as a function of the oligo. L. crispatus growth was supported by CLX01, CLX102, CLX109, CLX112, CLX116, CLX119, CLX115, CLX123, CLX125, CLX127, CLX128 and CLX130. L. rhamnosus growth was supported by CLX01, CLX105, CLX109, CLX112, CLX115, CLX121 and CLX131. Bif pseudocatenulatum growth was supported by CLX01, CLX103, CLX105, CLX109, CLX110, CLX111, CLX114, CLX117, CLX121, CLX122, CLX123, CLX125, CLX127, CLX128 and CLX130. Bif longum subsp. longum growth was supported by CLX01, CLX105, CLX109, CLX116, CLX119, CLX121, CLX122, CLX123, CLX127 and CLX128. Bif longum subsp. infantis growth was supported by CLX105, CLX109, CLX121, CLX127 and CLX128. Bif bifidum growth was supported by CLX115. Cl. butyricum growth was supported by CLX01, CLX102, CLX108, CLX109, CLX110, CLX111, CLX112, CLX116, CLX117, CLX119, CLX115, CLX121, CLX123, CLX125, CLX126 and CLX128. An example of selective growth is such of Cl. butyricum in CLX108; Its selectivity relies in the fact that the only microbe capable of utilizing CLX108 is Cl. butyricum and none of the other tested strains. The same selectivity was observed in CLX126 which only supported Cl. butyricum growth. The results are shown in Table 39.

TABLE 39
Summary of growth of selected probiotic strains with oligosaccharides generated here.

| | Supported growth of selected probiotic strains | | | | | | |
|---|---|---|---|---|---|---|---|
| | | B. longum | B. longum | B. | Clostridium | Lactobacillus | Lactobacillus |
| Oligo | B. bifidum | ssp. infantis | ssp.longum | pseudocatenulatum | butyricum | crispatus | rhamnosus |
| CLX101 | − | − | + | + | ++ | ++ | ++ |
| CLX102 | − | − | − | − | ++ | + | − |
| CLX103 | − | − | − | + | − | − | − |
| CLX105 | − | + | ++ | + | − | − | + |
| CLX107 | − | − | − | − | − | − | − |
| CLX108 | − | − | − | − | ++ | − | − |
| CLX109 | − | + | ++ | ++ | + | + | + |
| CLX110 | − | − | − | ++ | ++ | − | − |
| CLX111 | − | − | − | + | + | − | − |
| CLX112 | − | − | − | − | ++ | ++ | ++ |
| CLX113 | − | − | − | − | − | − | − |
| CLX114 | − | − | − | + | − | − | − |
| CLX116 | − | − | + | − | + | + | − |
| CLX117 | − | − | − | + | + | − | − |
| CLX119 | − | − | + | − | + | + | − |
| CLX115 | + | − | − | − | ++ | ++ | + |
| CLX121 | − | + | + | ++ | ++ | − | + |
| CLX115 | | | | | | | |
| CLX122 | − | − | + | ++ | − | − | − |
| CLX123 | − | − | + | + | + | + | − |
| CLX124 | − | − | − | − | − | − | − |
| CLX125 | − | − | − | + | + | + | − |
| CLX126 | − | − | − | − | + | − | − |
| CLX127 | − | + | + | + | − | ++ | − |
| CLX128 | − | + | + | + | + | ++ | − |
| CLX129 | − | − | − | − | − | − | − |
| CLX130 | − | + | − | + | − | + | − |
| CLX131 | − | − | − | − | − | − | + |

Symbols key:
−: <20%, +: 20-50%; ++: >50% growth relative to the positive control.

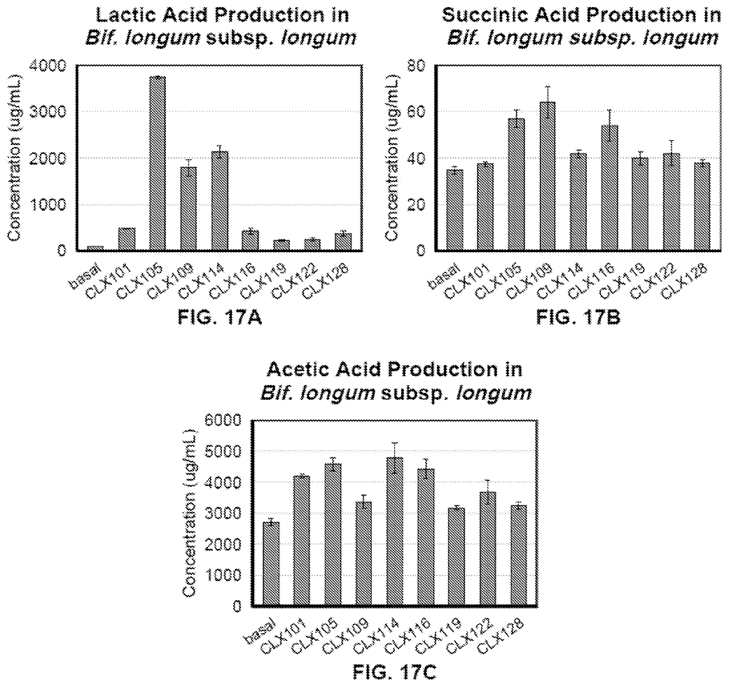

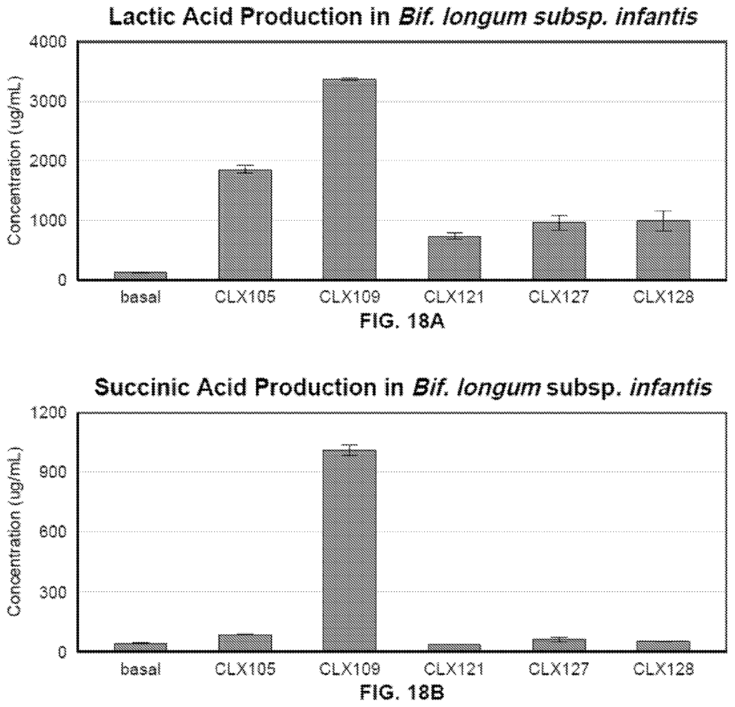

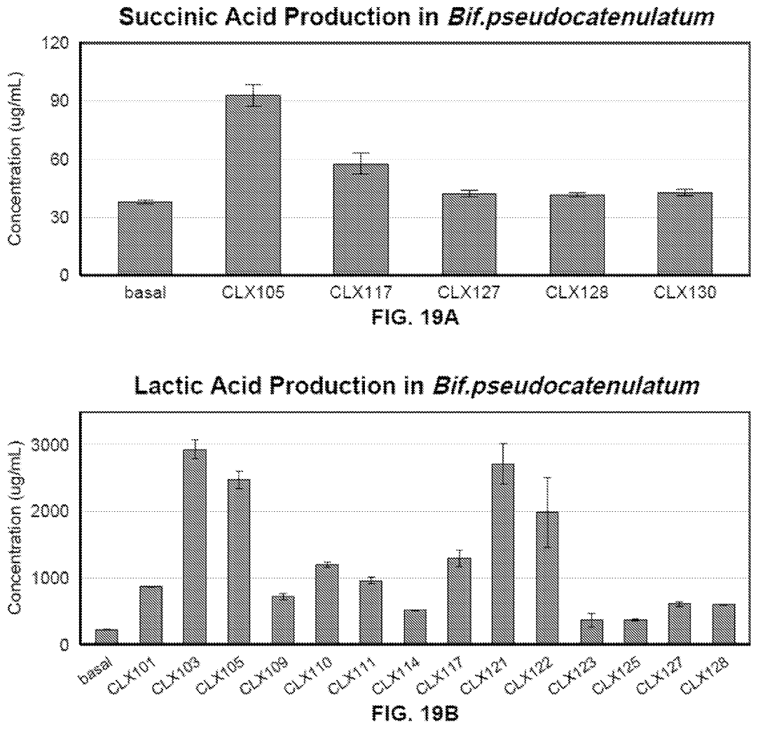

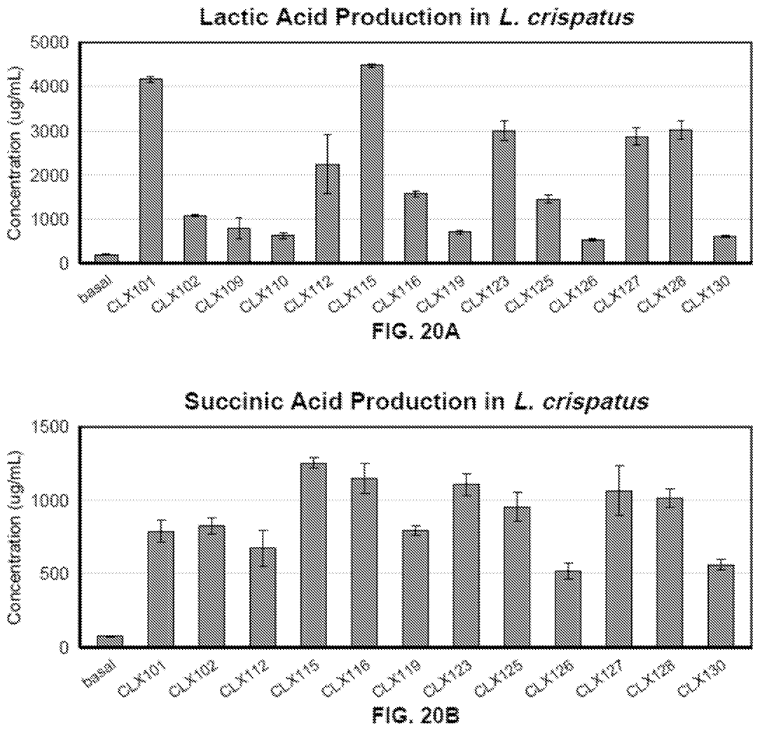

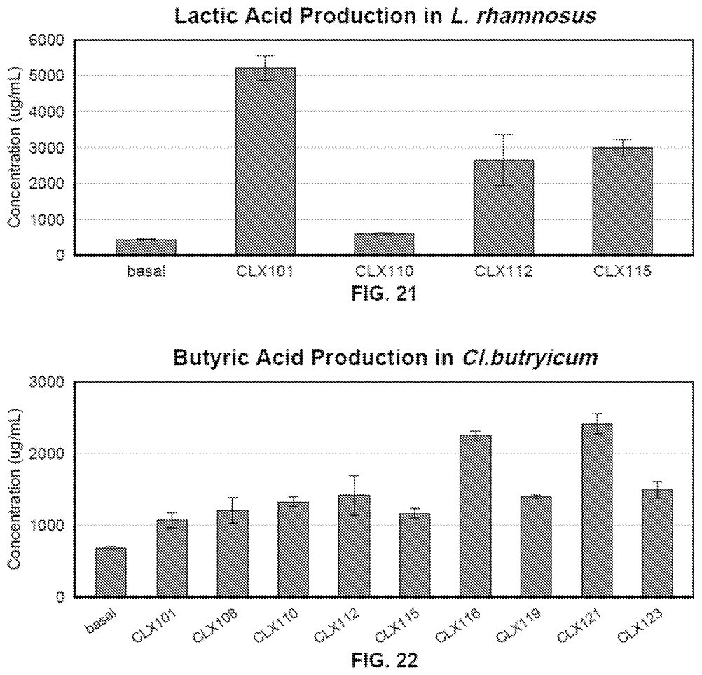

The selected microbial strains (Bif bifidum, Bif longum subsp. infantis, Bif longum subsp. longum, Bif pseudocatenatum) showed preferential growth with specific oligosaccharides. The growth resulted in the production of primarily lactic and/or acetic acids and to a lesser extent, the fermentation of certain oligos and strain combinations resulted in succinic acid production. All the described combinations produced more of each described acid than the corresponding acid produced in the basal samples (cultures with no supplemented glycans). Bif longum subsp. longum produced higher levels of lactic, acetic and/or succinic acids in CLX101, CLX105, CLX109, CLX114, CLX116, CLX119, CLX122, CLX128 compared to the control (basal sample) (FIG. 17A-C). Bif longum subsp. infantis produced lactic and succinic acids in CLX105, CLX109, CLX121, CLX127, and CLX128 (FIG. 18A-B). Notably, Bif longum subsp. infantis produced 10 times more succinic acid in composition CLX109 and produced more than 15 times more lactic acid in compositions CLX105 and CLX109 than the other oligosaccharides. Bif bifidum growth in CLX115 resulted in a higher OD than the basal control, however the levels of both lactic and acetic produced during the fermentation were comparable to the basal samples. Bif pseudocatenatum produced succinic acid in CLX105, CLX117, CLX127, CLX128 and CLX130 above the levels of the basal samples (FIG. 19A). Bif pseudocatenulatum produced lactic acid in CLX101, CLX103, CLX105, CLX109, CLX110, CLX111, CLX114, CLX117, CLX121, CLX122, CLX123, CLX125, CLX127 and CLX28 above the levels of the basal samples (FIG. 19B). L. crispatus produced lactic acid in CLX101, CLX110, CLX112, CLX115, CLX102, CLX109, CLX116, CLX119, CLX123, CLX125, CLX126, CLX127, CLX128 and CLX130. L. crispatus produced succinic acid in CLX101, CLX102, CLX126, CLX112, CLX116, CLX119, CLX115, CLX123, CLX125, CLX127, CLX128 and CLX130 (FIG. 20A-B). L. rhamnosus produced lactic acid in CLX101, CLX110, CLX112 and CLX115 but did not produce any succinic acid levels above the basal samples (FIG. 21). Cl. butyricum growth in different oligosaccharides resulted mainly in the production of butyric acid. Cl. butyricum produced butyric acid in CLX116, CLX119, CLX121 and CLX123 (FIG. 22). Butyric acid levels in CLX117 and CLX125 were not different from the basal samples despite the increase in OD during the fermentation.

These results indicate that, while many oligosaccharides can support the growth of certain bacterial strains, they each unique modulate the metabolic (SCFA) outputs of that bacteria. This allows the optimization of certain symbiotic pairings that are focused on metabolite modulation.

### Example 13

### (Effect of Oligosaccharides on Ex-Vivo Human Fecal Microbiota)

Fecal samples were collected from healthy donors by BioIVT and stored at −80° C. until processing. Aliquots of slurry from the fecal samples were prepared mixing three parts of fecal samples, one part of glycerol and one part of PBS. Slurries were stored at −80° C.

Static fecal fermentations were conducted in a deep 96-well format, under anaerobic conditions (Anaerobic Chamber Vinyl Type B), using a mix of gas (carbon dioxide 5%, hydrogen 5%, nitrogen balance), using inoculum from either individual donors or a pool of feces (2% of the fermentation mix). The following oligosaccharides were tested at a concentration of 0.6% w/v. The following oligos were tested: CLX101, CLX102, CLX103, CLX105, CLX108, CLX109, CLX110, CLX111, CLX112, CLX113, CLX114, CLX117, CLX115, CLX121, CLX122, CLX123, CLX124, CLX125, CLX126, CLX127, CLX128, CLX129, CLX130 and CLX131. Fermentation media was optimized to support diverse microbial taxa and control pH within the range of the colon physiological conditions, containing mineral and vitamin solution, CaCl₂ (10 mg/ml) and basic fermentation medium as described by MacFarlane G T et al (1989).

A mix of background sugars (xylan, amylopectin, potato starch, and pectin) were included in the basic fermentation media in low concentration to sustain microbial networks and minimize changes due to lack of nutrients which would confound the experimental results. Multiple samples were taken at different time points (0 h, 6 h, 11 h and 20 h) for monosaccharide analysis, oligosaccharide profiling, short chain fatty acid (SCFA) analysis and metabolomics analysis. After 20 hours of fermentation, gDNA was extracted from each well and sent for 16S rRNA sequencing. Four replicates were run for each oligo and untreated control.

DNA was extracted from fecal slurries using ZymoBIOMICS Kit D4308 and a KingFisher Flex DNA extraction robot. Microbial communities were profiled by sequencing the V4 region of the bacterial 16S rRNA gene amplified using 515F (5′-GTGCCAGCMGCCGCGGTAA-3′) and 806R (5′ GGACTACHVGGGTWTCTAAT-3′) primers. NovaSeq 6000 was used to obtain 250 bp paired end reads. Raw demultiplexed reads were processed using QJJME2 2020.11. Briefly, after quality checking, trimming, filtering and denoising were performed using the “dada2 denoise-paired” plugin in QJJME2. Taxonomic classification of ASVs was performed with the “q2-feature-classifier” plugin and a Naive Bayes classifier trained on Silva 138 99% OTUs from the 515F/806R region of 16S rRNA sequences. Finally, alpha diversity and beta diversity analyses were conducted with QJJME2 plugins and subsequently imported into MATLAB, R and Excel for further analysis.

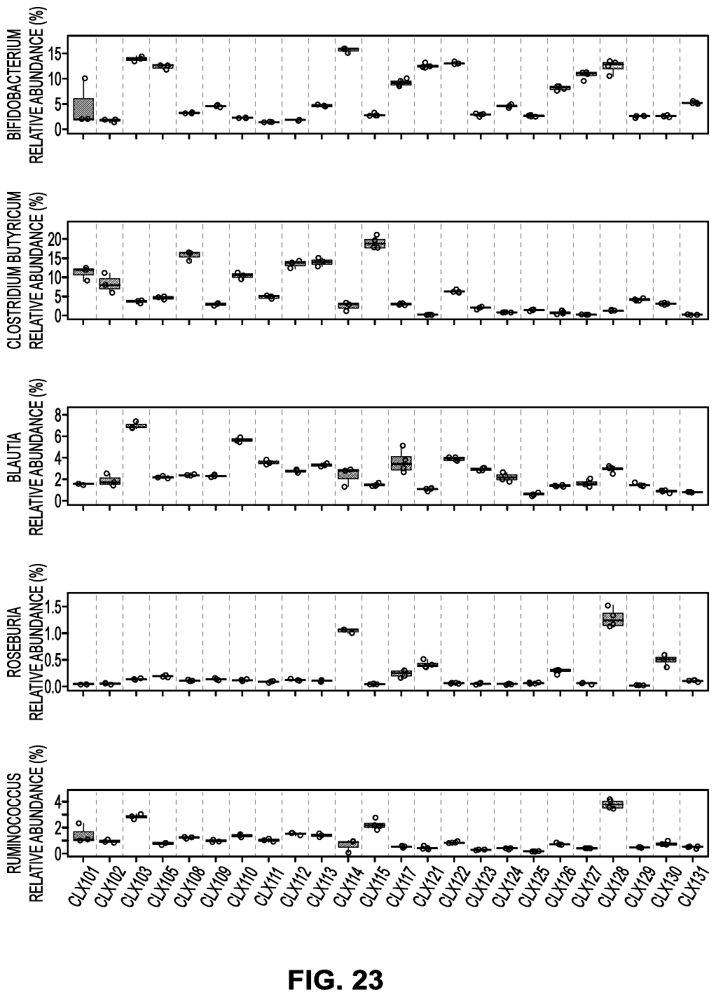

Each oligosaccharide generated a different impact in the microbial composition of the fecal sample compared to untreated sample (absence of oligosaccharide in the fecal fermentation medium) at phylum and species level, as shown in Table 40 and FIG. 23.

TABLE 40
Impact of tested oligosaccharides on selected bacteria (e.g., changes in relative
abundance of microbes in oligo-treated samples versus untreated control).

| | | Bifidobacterium | | Bacteoides | Parabacteroides | | Clostridium |
|---|---|---|---|---|---|---|---|
| Oligo ID | Bifidobacteria | animalis | Bacteroides | ovatus | distasonis | Firmicutes | butyricum |
| CLX101 | + | + | − | − | − | + | + |
| CLX102 | − | 0 | − | − | − | + | + |
| CLX103 | + | + | + | + | − | + | + |
| CLX105 | + | + | + | + | − | − | + |
| CLX108 | + | + | − | − | − | + | + |
| CLX109 | + | + | + | + | + | − | − |
| CLX110 | − | 0 | + | + | − | + | + |
| CLX111 | − | + | + | + | + | + | + |
| CLX112 | + | + | − | − | − | + | + |
| CLX113 | + | + | − | − | − | + | + |
| CLX114 | + | + | + | + | − | − | − |
| CLX117 | + | − | + | 0 | − | + | + |
| CLX115 | − | − | − | 0 | − | + | + |
| CLX121 | + | − | − | + | − | + | − |
| CLX122 | + | − | + | + | − | + | + |
| CLX123 | − | − | + | 0 | + | + | + |
| CLX124 | + | − | − | 0 | + | + | + |
| CLX125 | − | − | − | 0 | + | + | + |
| CLX126 | + | − | − | + | − | + | + |
| CLX127 | + | − | + | 0 | − | − | − |
| CLX128 | + | − | − | 0 | − | + | + |
| CLX129 | − | − | − | + | − | + | + |
| CLX130 | − | − | − | 0 | − | − | + |
| CLX131 | + | − | − | 0 | − | − | − |
| | | Ruminococcus | Ruminococcus | | | | |
| Oligo ID | Ruminococcus | gnavus | torques | Blautia | Roseburia | Faecalibacterium | Proteobacteria |
| CLX101 | 0 | + | 0 | + | + | − | − |
| CLX102 | 0 | − | 0 | + | + | − | − |
| CLX103 | 0 | + | + | + | + | + | − |
| CLX105 | 0 | − | + | + | + | + | − |
| CLX108 | 0 | + | 0 | + | + | + | − |
| CLX109 | 0 | + | + | + | + | + | − |
| CLX110 | 0 | + | 0 | + | + | + | − |
| CLX111 | 0 | + | + | + | + | + | − |
| CLX112 | 0 | + | 0 | + | + | + | − |
| CLX113 | 0 | + | + | + | + | + | − |
| CLX114 | 0 | + | + | + | + | + | − |
| CLX117 | 0 | + | + | + | + | + | − |
| CLX115 | 0 | + | − | + | + | 0 | − |
| CLX121 | 0 | − | + | + | + | + | − |
| CLX122 | 0 | + | − | + | + | + | − |
| CLX123 | 0 | − | + | + | + | + | − |
| CLX124 | + | − | + | + | + | 0 | − |
| CLX125 | 0 | − | + | + | + | + | − |
| CLX126 | 0 | + | + | + | + | + | − |
| CLX127 | + | − | + | + | + | + | − |
| CLX128 | + | + | + | + | + | + | − |
| CLX129 | 0 | + | − | + | + | 0 | + |
| CLX130 | 0 | + | − | + | + | + | + |
| CLX131 | 0 | + | + | + | + | 0 | + |

Key symbols:
+ significant increase in relative abundance vs untreated control; − significant decrease in relative abundance vs untreated control; 0 no change in relative abundance vs untreated control.

We evaluated the effect of the oligos in different taxa and species of bacteria with potential impact on host health. Bifidobacterium, a well-known genus associated with beneficial health effects and positive impacts on gut mucosal barrier, was increased by CLX101, CLX103, CLX105, CLX108, CLX109, CLX112, CLX113, CLX114, CLX117, CLX121, CLX122, CLX124, CLX126, CLX127, CLX128, CLX131. Most of the increase was associated to the species Bifidobacterium pseuodcatenulatum or animalis, whose relative abundance was significantly higher in the presence of the aforementioned substrates compared to the untreated control. In addition, CLX111 also increased Bifidobacterium animalis species.

The genus Bacteroides, a major commensal within the human gut and a mucin and plant material consumer, was increased by CLX103, CLX105, CLX109, CLX110, CLX111, CLX114, CLX117, CLX122, CLX123 and CLX127. The species B. ovatus was enriched in the presence of CLX103, CLX105, CLX109, CLX110, CLX111, CLX114, CLX121, CLX122, CLX126 and CLX129. Parabacteroides distasonis, a species of bacteria in the same family as Bacteroides, was increased only by CLX109, CLX111, CLX123, CLX124, CLX135, which shows that modulation of the complex community could be performed with precision as a function of the oligosaccharide used in the fecal fermentation.

Firmicutes were enriched by all the tested oligos, with the exception of CLX105, CLX109, CLX114, CLX127, CLX130, and CLX131. High butyric acid producer, Cl. butyricum, was also enriched by all of the tested oligos, excluding CLX 109, CLX114, CLX121, CLX127, and CLX131. Compared to all of the tested oligos, CLX115 enriched Cl. butyricum the most compared to the untreated control (FIG. 23). Ruminococcus genus from the Ruminococcaceae family increased with CLX124, CLX127 and CLX128. Ruminococcus gnavus (Lachnospiraceae family) species were increased with all the tested oligos with the exception of CLX102, CLX105, CLX121, CLX123, CLX124, CLX125 and CLX127. Ruminococcus torques (Lachnospiraceae family) also increased with CLX103, CLX105, CLX109, CLX111, CLX113, CLX114, CLX117, CLX121, CLX123, CLX124, CLX125, CLX126, CLX127, CLX128 and CLX131. Blautia was increased by all the tested oligos, with CLX103 creating the highest change compared to untreated control (FIG. 23). Roseburia increased by all oligos, with CLX114 increasing this genus the most (FIG. 23). Relative abundance of Faecalibacterium was increased by all tested oligos except CLX101, CLX102, CLX115, CLX124, CLX129 and CLX131 (Table 40). Most of the tested oligos decreased Proteobacteria phylum compared to the untreated control. (Table 40).

Supernatants of fecal bacterial growths were derivatized with 2-nitrophenylhydrazine (2-NPH) for LC-MS/QTOF analysis. In brief, 20 μL of supernatant was added to 20 μL of N-(3-Dimethylaminopropyl)-N′-ethylcarbodiimide hydrochloride (1-EDC HCl) in 5% pyridine. Then 40 μL of 200 mM 2-NPH in 80% acetonitrile with 50 mM HCl was added and briefly vortexed prior to incubating for 30 minutes at 40° C. After incubation, samples were diluted with 400 μL of 10% acetonitrile and vortexed. The dilutions were centrifuged prior to LC-MS/QTOF analysis.

Short chain fatty acids were analyzed with a 1290 Infinity II LC (Agilent Technologies, Santa Clara, CA) equipped with a reverse phase column (Zorbax Eclipse C18 2.1×50 mm; Agilent Technologies, Santa Clara, CA) and 6530 LC-MS QTOF (Agilent Technologies, Santa Clara, CA). LC separation was performed with 5% acetonitrile with 0.1% formic acid (solvent A) and 95% acetonitrile with 0.1% formic acid (solvent B). A separation gradient was as follows: 5% to 15% B for 2 minutes, then 15% to 30% B for 3 minutes, 30-100% B in 0.1 minute, hold at 100% B for 1.9 minutes, return to 5% B in 0.1 minute, and equilibrate at 5% B for 1.9 minutes. The MS conditions were set to positive mode with a scan range set at m/z 50-1100 at 1 spectra/sec scan rate. Peak area was quantitated using Agilent Quantitative Analysis software and areas were normalized to internal standards and compared to an external standard curve for quantitation.

Supernatants of fecal bacterial growths were diluted with acetonitrile (1:10 dilution) for LC-MS/QTOF analysis. Metabolites were analyzed with a 1290 Infinity II LC (Agilent Technologies, Santa Clara, CA) equipped with a HILIC column (InfinityLab Poroshell 120 HILIC-Z, 2.1×150 mm; Agilent Technologies, Santa Clara, CA) and 6530 LC-MS QTOF (Agilent Technologies, Santa Clara, CA). LC separation was performed with 10% 200 mM ammonium formate with 0.1% Formic Acid+90% Water (solvent A) and 10% 200 mM ammonium formate with 0.1% Formic Acid+90% Acetonitrile (solvent B). The MS conditions were set to positive mode with a scan range set at m/z 50-1700 at 1 spectra/sec scan rate. Peak area was quantitated using Agilent Quantitative Analysis software and areas were normalized to internal standards for quantitation.

The microbial short chain and organic acids were monitored during the fecal fermentation at time points 6-hour, 10-hour, and 20-hour and were compared to a sample containing only background sugars. Short chain and organic acids make up the key intermediate and products of carbohydrate fermentation, many of which are known to be beneficial for human health. Butyrate is an important metabolite linked to intestinal barrier function and is a key modulator of the immune system. However, not all end-product metabolites reach their final state in the same way. Butyrate can be produced through a pathway with lactic acid as an intermediate. Lactic acid production may indicate differential metabolism within an organism or enrichment in certain communities that employ this cycle.

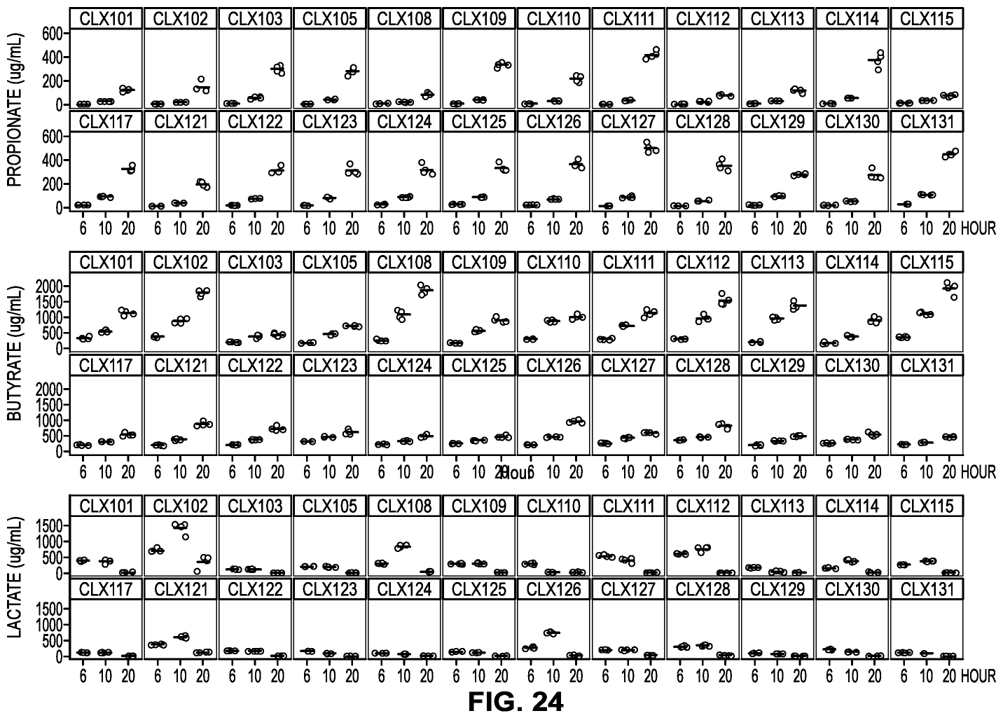

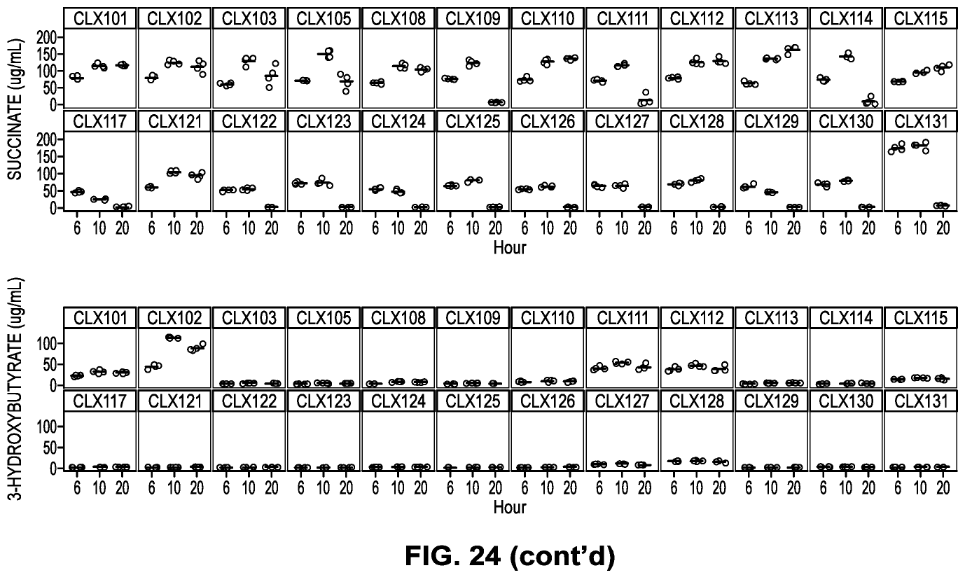

All oligosaccharides (CLX101, CLX102, CLX103, CLX105, CLX108, CLX109, CLX110, CLX 111, CLX 112, CLX113, CLX114, CLX117, CLX115, CLX121, CLX122, CLX123, CLX124, CLX125, CLX126, CLX127, CLX128, CLX129, CLX130 and CLX131CLX115A) produced propionic, butyric, lactic and/or succinic acid, as seen in Table 41. Propionic acid levels in CLX115 and CLX121, Lactic acid levels in CLX117, CLX124, CLX129 and CLX131, and succinic acid in CLX117 were not different than the levels produced in the untreated control fermentation. The organic acid 3-hydroxybutyric acid was only produced by CLX101, CLX102, CLX111, CLX112, CLX115, CLX127 and CLX128. Propionic Acid growth by the 20-hour timepoint in CLX103, CLX105, CLX109, CLX111 and CLX114 were approximately three times larger than the other oligosaccharides. Butyric acid growth were similar for all oligosaccharides at the 6-hour timepoint. However, by the 20-hour time point CLX102, CLX108, CLX112, CLX113 and CLX115 were the top producers of butyric acid. For all oligosaccharides, lactic acid production is observed in the 6-hour timepoint. Some oligosaccharides (CLX102, CLX108, CLX112, CLX114, CLX115, CLX121 and CLX126) continued to produce lactic acid to peak at the 10-hour timepoint. However, lactic acid production peaked at the 6-hour timepoint for CLX101, CLX103, CLX105, CLX109, CLX110, CLX111, CLX113, CLX117, CLX122, CLX123, CLX124, CLX129, CLX130, CLX131, and decreased to the 20-hour timepoint. Succinic acid growth peaked at the 10-hour timepoint for CLX102, CLX103, CLX105, CLX108, CLX111, CLX115A, CLX121, CLX122, CLX123, CLX126, CLX128, CLX130 and CLX131, while CLX101, CLX110, CLX112, CLX113 and CLX115 peaked at the 20-hour timepoint. Production of 3-hydroxybutyric acid were similar between all timepoints for most of the oligosaccharides. However, 3-hydroxybutryic acid production in CLX102 more than doubled from the 6 to 10-hour timepoints followed by a decrease observed in the 20-hour timepoint. Similarly, 3-hydroxybutryic acid production for CLX115 and CLX128 increased seven-fold in the first 6 hours. Selected results are shown in Table 41 and FIG. 24.

TABLE 41
Production of Short Chain and Organic Acids in Oligosaccharides

| | Production of Short Chain and Organic Acids | | | | |
|---|---|---|---|---|---|
| | Propionic | Butyric | Lactic | Succinic | 3-hydroxybutyric |
| Oligo | Acid | Acid | Acid | Acid | Acid |
| CLX101 | + | + | + | + | + |
| CLX102 | + | + | + | + | + |
| CLX103 | + | + | + | + | − |
| CLX105 | + | + | + | + | − |
| CLX108 | + | + | + | + | − |
| CLX109 | + | + | + | + | − |
| CLX110 | + | + | + | + | − |
| CLX111 | + | + | + | + | + |
| CLX112 | + | + | + | + | + |
| CLX113 | + | + | + | + | − |
| CLX114 | + | + | + | + | − |
| CLX117 | + | + | − | − | − |
| CLX115 | − | + | + | + | + |
| CLX121 | − | + | + | + | − |
| CLX122 | + | + | + | + | − |
| CLX123 | + | + | + | + | − |
| CLX124 | + | + | − | + | − |
| CLX125 | + | + | + | + | − |
| CLX126 | + | + | + | + | − |
| CLX127 | + | + | + | + | + |
| CLX128 | + | + | + | + | + |
| CLX129 | + | + | − | + | − |
| CLX130 | + | + | + | + | − |
| CLX131 | + | + | − | + | − |

Key symbols: + = growth trend − = no growth trend

Utilization of choline and methionine were decreased by select oligosaccharides. The choline levels in CLX101, CLX102, CLX103, CLX105, CLX108, CLX109, CLX111, CLX112, CLX113, and CLX114 were higher than those of untreated samples (FIGS. 25A-25B). The higher choline levels indicate the reduced use of choline than with other oligosaccharides. Methionine levels in CLX101, CLX110, CLX111 and CLX112 increased from a 0-hour timepoint (FIGS. 25A-25B). The results suggest that CLX101, CLX102, CLX103, CLX105, CLX108, CLX109, CLX111, CLX112, CLX113, CLX114, CLX117, CLX115, CLX122, CLX124, CLX125, CLX126, CLX127, CLX128, CLX129, CLX130, CLX131 may have potential in improving intestinal barrier functioning. These oligosaccharides increase one or more of: the abundance of bacterial beneficial to the intestinal barrier (namely Bifidobacterium and butyrate producers such as Cl. Butyricum, Blautia, Rosebaria and Faecalibacterium), the production of acetate and butyrate which improve the intestinal barrier and regulate inflammation, the production of GABA which regulates inflammation, and certain amino acids associated with mucin production (e.g., proline). The oligosaccharides also reduce abundance of proteobacteria which is associated with impaired intestinal barrier.

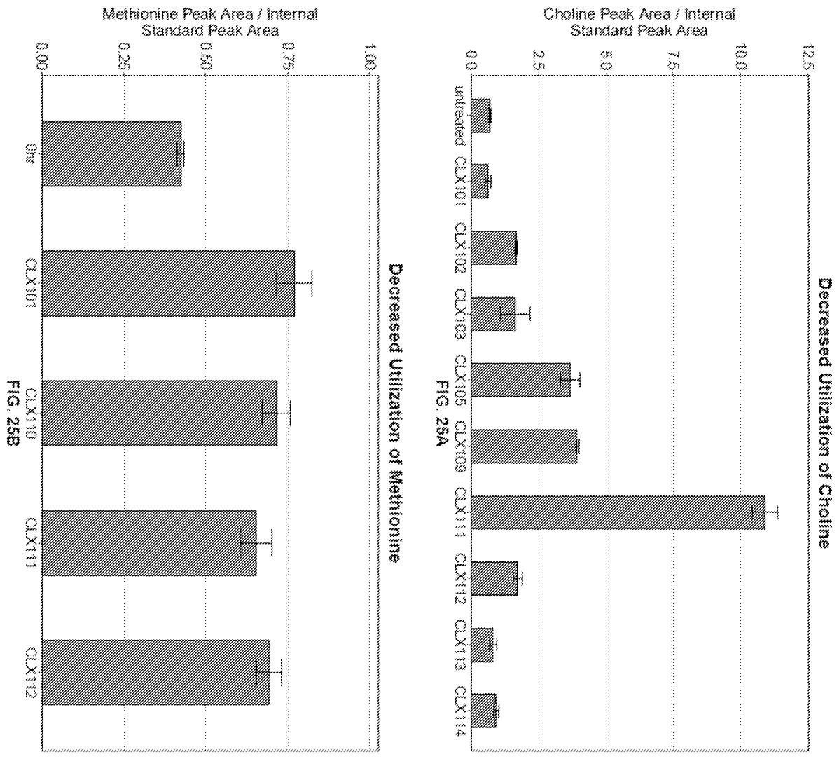

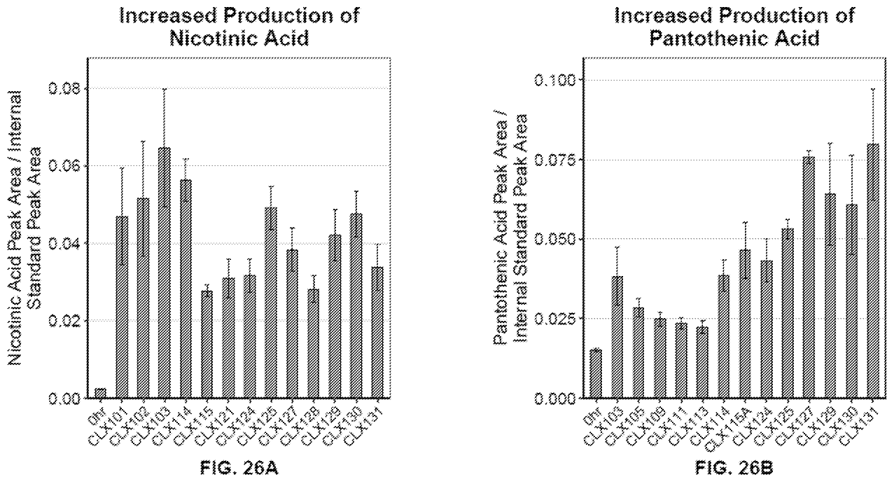

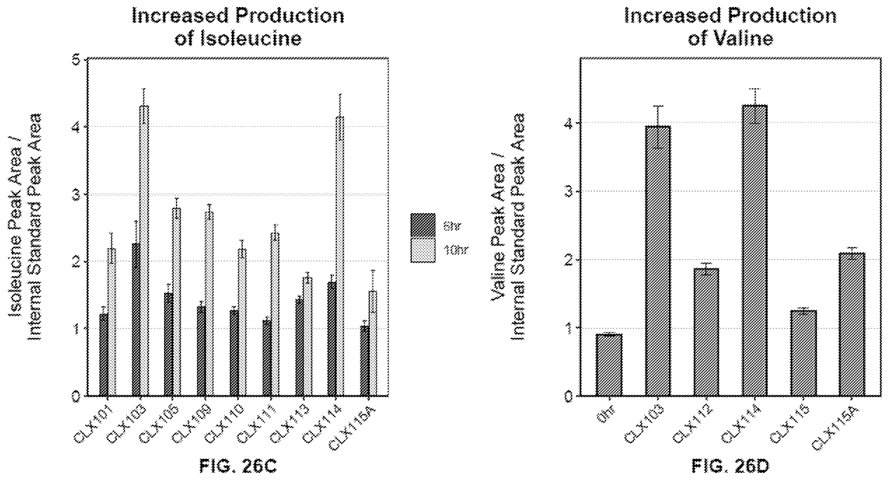

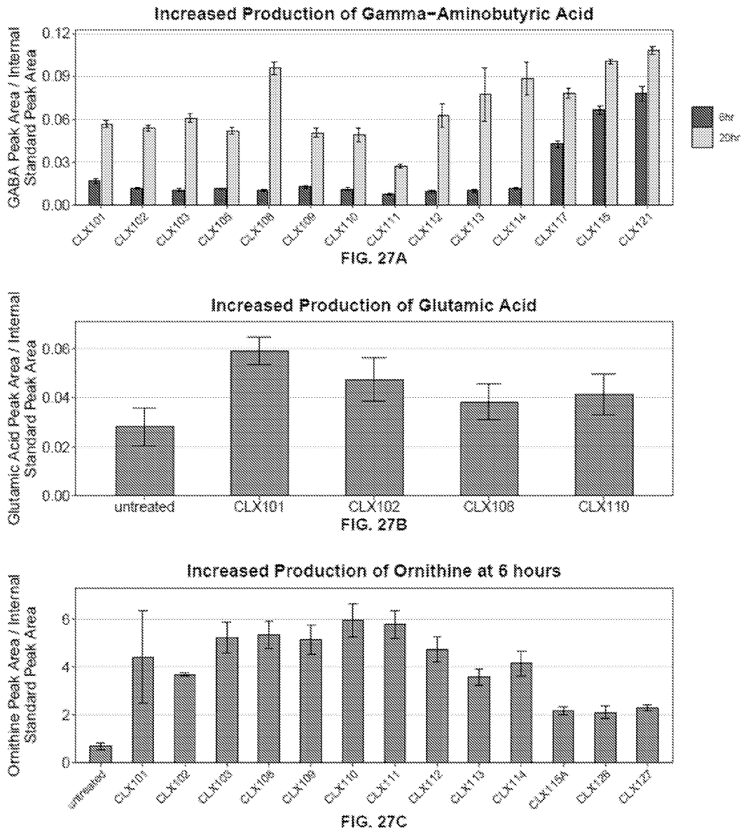

Over time nicotinic acid, pantothenic acid, isoleucine, valine, gamma-aminobutyric acid, glutamic acid and ornithine were produced in select oligosaccharides (FIGS. 26A-26D and FIGS. 27A-27C). Nicotinic acid production in CLX101, CLX102, CLX103, CLX114, CLX115, CLX121, CLX124, CLX125, CLX127, CLX128, CLX129, CLX130 and CLX131 increased from a 0-hour to the 20-hour timepoint. The nicotinic acid levels increased by at least 20× between the 2 timepoints for the select oligosaccharides. Pantothenic acid production in CLX103, CLX105, CLX109, CLX111, CLX113, CLX114, CLX115A, CLX124, CLX125, CLX127, CLX129, CLX130 and CLX131 increased significantly by 6-hour and/or 20-hour timepoints. Isoleucine production in CLX101, CLX103, CLX105, CLX109, CLX110, CLX111, CLX113, CLX114 and CLX115A increased between 6-hour and 10-hour timepoints. Valine production in CLX103, CLX112, CLX114 and CLX115 increased after the 0-hour timepoint. CLX115A Valine levels accumulated overtime duplication the concentration detected at the beginning of the fermentation. Gamma-aminobutyric acid production increased between 6-hour and 20-hour timepoints. Glutamic Acid production increased over time in comparison to the untreated samples in CLX 101, CLX102, CLX108 and CLX110. Ornithine production increased by time 6-hours beyond levels in the untreated samples in CLX101, CLX102, CLX103, CLX108, CLX109, CLX110, CLX111, CLX112, CLX113, CLX114, CLX115A, CLX126 and CLX127.

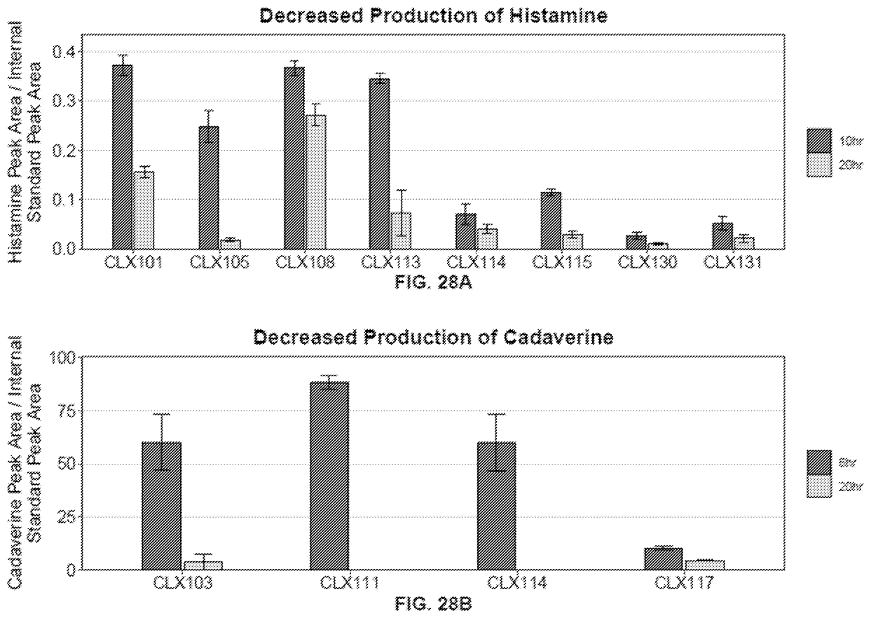

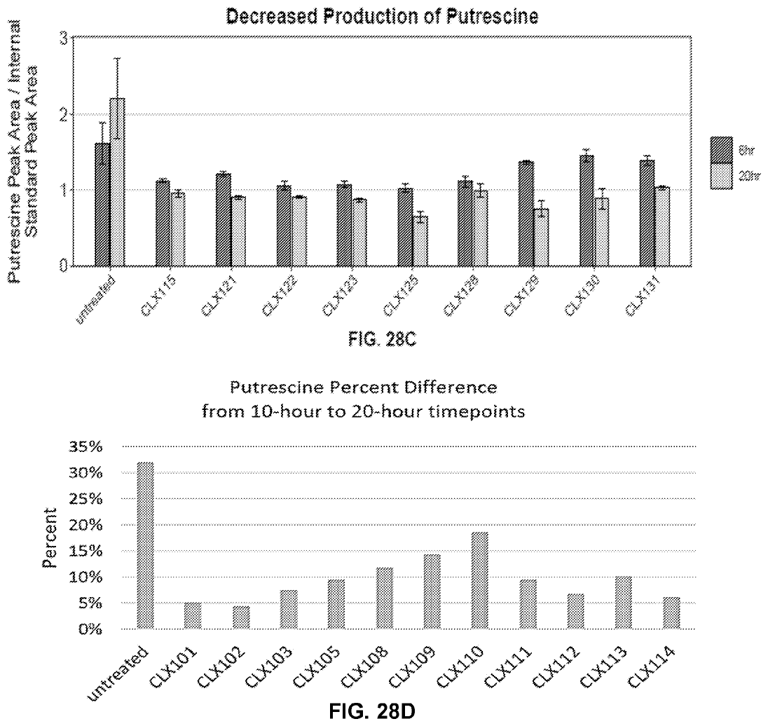

Over time histamine, cadaverine and putrescine levels decreased in select oligosaccharides (FIGS. 28A-28D). Histamine levels decreased from the 10-hour to 20-hour timepoint in CLX101, CLX105, CLX108, CLX113, CLX114, CLX115, CLX130 and CLX131. Cadaverine levels decreased from the 6-hour to 20-hour timepoint in CLX103, CLX11, CLX114 and CLX117. Putrescine levels continuously decreased after 6-hour in CLX115, CLX121, CLX122, CLX123, CLX125, CLX128, CLX129, CLX130, CLX131. Furthermore, the percent change difference in putrescine levels between the 10-hour and 20-hour timepoints were less for CLX101, CLX102, CLX103, CLX105, CLX108, CLX109, CLX110, CLX111, CLX112, CLX113. CLX114, and CLX117 than the untreated samples. This lower percent change difference indicates the decrease in putrescine production by these select oligosaccharides.

The results also suggest that oligosaccharides CLX101, CLX102, CLX103, CLX105, CLX108, CLX109, CLX110, CLX111, CLX112, CLX113, CLX114, CLX115, CLX121, CLX122, CLX123, CLX125, CLX127, CLX128, and CLX130 are good candidates for therapeutic applications involving improvement of kidney functioning. These oligosaccharides increase Bifidobacteria which improve barrier function and decrease relative abundance of species that are capable of inducing local and systemic inflammation like Proteobacteria (Kambay et al. 2018). Moreover, these oligosaccharides promote production of SCFA or GABA, which are anti-inflammatory substances associated to CKD disease (Wong et al. 2014, Barrett et al. 2012).

The results also suggest that oligosaccharides CLX101, CLX102, CLX103, CLX105, CLX108, CLX109, CLX110, CLX111, CLX112, CLX113, CLX114, CLX119, CLX115, CLX121, CLX122, CLX123, CLX125, CLX126, CLX127, CLX128, CLX129, CLX130 and CLX131 are good candidates for therapeutic applications involving improvement of conditions related to gut-brain interactions. These oligosaccharides can increase the production of SCFA, which play an important role in lowering systemic inflammation that can lead to reduced neuroinflammation (Needham et al. 2020). Furthermore, the SCFA results with these oligosaccharides has been linked with an increase in Bifidobacteria, connected to reduced LPS levels, that can lead to reduction of neuroinflammation and improve CNS related indications (Kim et al 2018). Metabolism of glutamate in the presence of the oligosaccharides, increased GABA levels, which is inverse related to CNS indications like depression, bipolar disorder, schizophrenia or ASD (Needham et al. 2020). The results also suggest that oligosaccharides CLX101, CLX102, CLX103, CLX105, CLX108, CLX109, CLX110, CLX111, CLX112, CLX113, CLX114, CLX119, CLX115, CLX121, CLX122, CLX123, CLX125, CLX126, CLX127, CLX128, CLX129, CLX130 and CLX131 are good candidates for therapeutic applications involving reduction of allergic symptoms. These oligosaccharides increase SCFA, which are key drivers of T-cell subset proliferation and activity, which is related to normal immunity (Luu et al. 2020). The results also show that Bifidobacteria increases, which can influence childhood allergic sensitization (Lynch 2016).

### Example 14

### (Effect of Oligosaccharides Versus Polysaccharides on Ex-Vivo Human Fecal Microbiota)

Static fecal fermentations were performed to determine advantages of oligosaccharides versus polysaccharides to modify major microbial group. In vitro fermentation of fecal samples, gDNA extraction and 16s rRNA sequencing and analysis was performed as described in Example 3. Oligosaccharide CLX109 increased the relative abundance of Faecalibacterium, Bacteroides, Bacteroides ovatus, Parabacteroides diastonis and Ruminococcus torques, while the source polysaccharide did not create a significant effect in these group of microorganisms (Table 42). Oligosaccharide CLX112 increased relative abundance of Roseburia and Faecalibacterium, while the source polysaccharide did not (Table 42). Oligosaccharide CLX105 induced an increase in the relative abundance of Faecalibacterium compared to the control. In contrast, Faecalibacterium levels in CLX105 source polysaccharide were not significantly different to the control (Table 42). In general, the results indicate the oligosaccharides outperform the polysaccharides in terms of increasing the abundance of beneficial bacteria.

Oligosaccharide CLX108 increased the relative abundance of Roseburia and Faecalibacterium, while source polysaccharide did not. When compared to the source polysaccharide the oligosaccharide CLX111 increased the relative abundance of Roseburia, Feacalibacterium, Bacteroides, Parabacteroides diastonis, Ruminococcus torques. (Table 42). Oligosaccharide CLX113 increased the relative abundance of Bifidbacterium, Feacalibacterium, Ruminococcus torques, while the source polysaccharide did not. Oligosaccharide CLX114 increased the relative abundance of Faecalibacterium, Ruminococcus, and species of Bacteroides, while source polysaccharide did not (Table 42).

TABLE 42
Changes in relative abundance of microbes in oligo or poly-treated
samples versus untreated control. Oligos identified by CLX ID, corresponding
polysaccharides indicated by CLX ID plus “_P”.

| | Bifidobacterium | | | Bacteroides | Parabacteroides |
|---|---|---|---|---|---|
| Oligo ID | animalis | Bacteroidota | Bacteroides | ovatus | distasonis |
| CLX105 | + | + | + | + | − |
| CLX105_P | + | + | + | + | − |
| CLX108 | + | − | − | − | − |
| CLX108_P | + | − | − | − | − |
| CLX109 | + | + | + | + | + |
| CLX109_P | + | − | − | − | − |
| CLX111 | + | + | + | + | + |
| CLX111_P | + | − | − | + | − |
| CLX112 | + | − | − | − | − |
| CLX112_P | + | − | − | + | − |
| CLX113 | + | − | − | − | − |
| CLX113_P | − | − | − | + | − |
| CLX114 | + | + | + | + | − |
| CLX114_P | + | − | − | + | − |

| | Ruminococcus | | | |
|---|---|---|---|---|
| Oligo ID | torques | Blautia | Roseburia | Faecalibacterium |
| CLX105 | + | + | + | + |
| CLX105_P | + | + | + | − |
| CLX108 | − | + | + | + |
| CLX108_P | − | + | − | − |
| CLX109 | + | + | + | + |
| CLX109_P | − | + | + | − |
| CLX111 | + | + | + | + |
| CLX111_P | − | + | − | − |
| CLX112 | − | + | + | + |
| CLX112_P | − | + | − | − |
| CLX113 | + | + | + | + |
| CLX113_P | − | + | + | − |
| CLX114 | + | + | + | + |
| CLX114_P | − | + | + | − |

Key symbols:
+ = significant increase in relative abundance vs untreated control; − significant decrease in relative abundance vs untreated control.

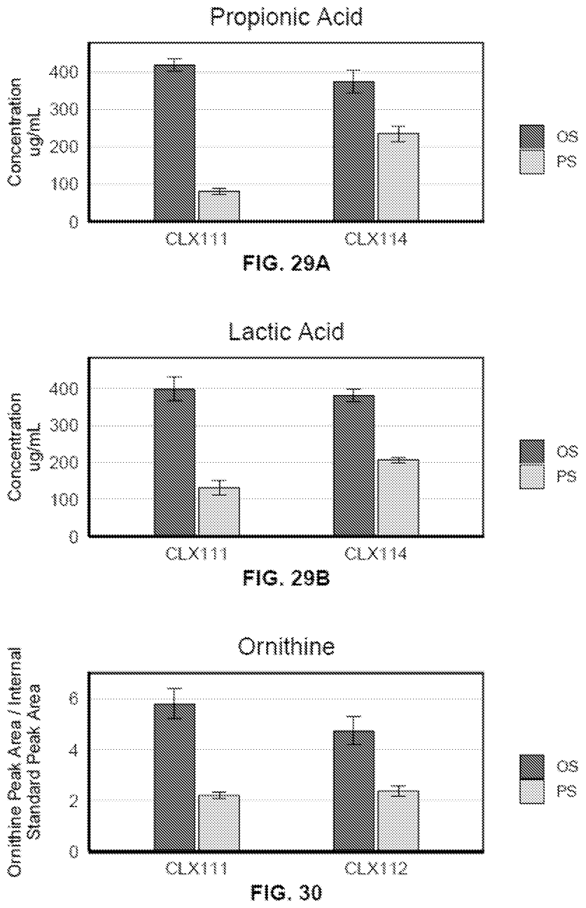

Results indicate that the oligosaccharides modulate individual microbes within the complex community differently than their source polysaccharide, suggesting different functionalities of the oligo versus polysaccharides. Oligosaccharides CLX111 and CLX114 indicated greater concentrations of the organic acids, propionic and lactic (FIG. 29A-29B) than the corresponding polysaccharides. Additionally, CLX111 and CLX112 produced higher levels of ornithine (FIG. 30), suggesting microbial preferences for the oligosaccharides than the corresponding polysaccharides.

### Example 15

### (Evaluation of CLX 112 as Prebiotic for Lactobacillus Rhamnosus)

Benefits of Lactobacillus rhamnosus as probiotics may be synergistically increased by combining a prebiotic agent. Based in our single microbe growth results, using our oligos, we determined that CLX112 may be a prebiotic agent for enhancing growth and colonization of Lactobacillus Rhamnosus. Moreover, the growth of L. rhamnosus on CLX112, surpasses the effect of the source polysaccharide.

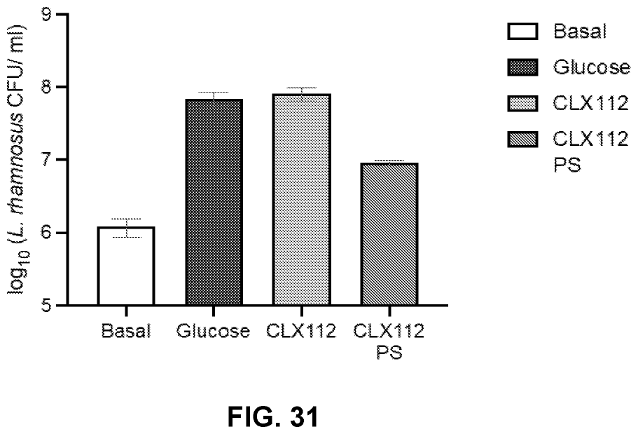

To determine if CLX112 supports the growth better than the corresponding polysaccharide, Lactobacilllus rhamnosus was incubated in minimum media MRS with a final concentration of CLX112 or polysaccharide of 1% w/v, as described in Example 13. Growth was monitored by serially diluting samples of the growing culture, plating on MRS agar at 24 hours and counting the grown colonies. Results show stronger growth of L. rhamnosus on CLX112 than on the corresponding polysaccharide (FIG. 31).

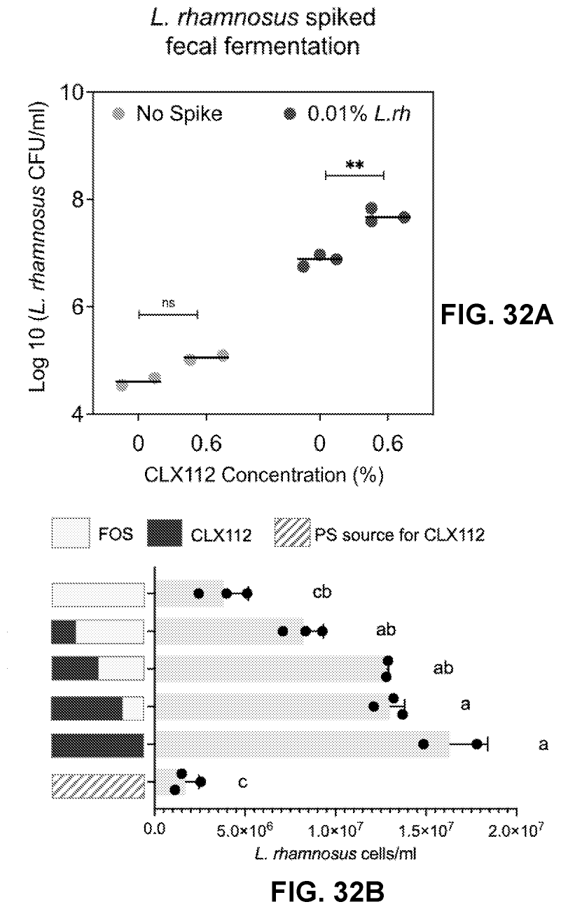

To evaluate, if the enrichment of Lactobacillus rhamnosus with CLX112 occurs in the context of a complex microbial community, fecal fermentation was conducted in the presence of CLX112 and L. rhamnosus. Fermentation media was inoculated with 1% activated fecal slurry and 0.01% pure culture of L. rhamnosus and incubated under anaerobic conditions (as described in Example 13). As controls, we run the fecal fermentations in the absence of L. rhamnosus with or without CLX112 supplementation. As a result, a total of 4 conditions were tested (FIG. 32A). After 24 hours microbial DNA was extracted (QIAamp PowerFecal Pro DNA Kit, QIAGEN) and qPCR was used to quantify the total load of Lactobacillus rhamnosus species. Quantitative real-time PCR (qPCR) was used to quantify the total load of Lactobacillus rhamnosus species. To run the qPCR, Power SYBR Green PCR Master Mix (Applied Biosystems) was used along with primers LactoR (5′-GTCCATTGTGGAAGATTCCC-3′) and LrhamF (5′-TGCTTGCATCTTGATTTAATTTTG-3′) at a concentration of 0.6 pMol/uL(Byun, Madkarni et al. 2004). The primers amplified the V1 region in the L. rhamnosus 16S rRNA gene, specific for this species. To quantify, a standard curve was prepared by creating serial dilutions of extracted DNA from a pure culture of L. rhamnosus and run alongside the spiked fermentation samples.

Results showed that the enrichment of L. rhamnosus is greater when CLX112 supplemented. In the absence of CLX112, levels of L. rhamnosus are 10 times (1 log) lower than those detected in the fermentation were CLX112 is supplemented. Furthermore, native L. rhamnosus species are not enriched in the presence of CLX112 which suggests the effect of CLX112 is strain specific (FIG. 32A).

A supplementary experiment was design to test whether the enrichment of L. rhamnosus is CLX112 oligosaccharide specific or can be achieved by the supplementation of other oligosaccharide fractions. For this purpose, multiple fermentations spiked with 0.01% L. rhamnosus were supplemented with different ratios of CLX112 and fructooligosaccharides (FOS). Previous results had suggested that L. rhamnosus is not capable of growing in FOS as the sole carbon source. All fermentations were supplemented with a total of 0.6% w/v oligosaccharides. The tested ratios were 0+0.6, 0.1+0.5, 0.3+0.3, 0.5+0.1, 0.6+0% w/v pf CLX112 and FOS correspondingly. (FIG. 32B). Additionally, fermentation in the presence of the polysaccharide (PS) source of CLX112 was run to compare it effect compared to its oligosaccharide counterparts.

Enrichment was not significant in the exclusive presence of FOS and the polysaccharide source of CLX112. There is a correlation between the concentration of CLX and the absolute levels of L. rhamnosus. Only when CLX112 exceed 0.5% w/v L. rhamnosus was significantly enriched. In fact, levels of L. rhamnosus were 4 and 9 times higher in the presence of CLX112 when compared with FOS and polysaccharide.

Results support that CLX112 is a fiber that can be used in combination with Lactobacillus rhamnosus in a symbiotic formulation due to its ability to enrich this strain in the context of complex communities and the fact that the interaction between L. rhamnosus and CLX112 seems to be strain and oligosaccharide specific.

### Example 16

### (Evaluation of Oligosaccharide Solubility and Viscosity)

Solubility and viscosity are important for formulation of food ingredients, with high solubility and tunable viscosity characteristics being preferred for fluid applications. Generally, polysaccharide rich materials have high viscosity, low solubility, or both, making them unsuitable for fluid applications without significantly affecting the organoleptics of a product. In providing a method to create oligosaccharides from a beneficial polysaccharide source, we provide the ability to deliver high concentrations of beneficial compounds from polysaccharides to the gut microbiome without significantly modifying the clarity or viscosity of a product.

Solubility: 20%, 10%, and 5% (w/w) solutions of oligosaccharide in water were incubated at 25° C. for 10 minutes with agitation and evaluated for solubility and color using qualitative metrics.

Viscosity: 10%, 5%, and 1% (w/w) solutions of oligosaccharide in water were incubated at 25° C., and the absolute viscosity was measured using a microVISC-m viscometer from Rheosense in automatic mode. The shear stress was measured across a range of shear rates and verified to be linear (a Newtonian fluid) in the measured regime. The relationship η=τ/\dot{γ}, where η is the absolute (dynamic) viscosity (mPa·s), γ is the shear rate (s⁻¹), and r is the shear stress (mPa), was used to solve for the dynamic viscosity of the oligosaccharide at a given temperature and concentration.

Results: Generation of oligosaccharides from polysaccharides resulted in materials that were generally soluble or formed colloidal suspensions at concentrations ≥20% (w/w). The absolute viscosity of the resulting oligosaccharide solutions was low (≤7 mPa·s) and did not rapidly increase, even at concentrations up to 10% (w/w). The solubility and viscosity of the oligosaccharides described here are summarized in Table 43.

TABLE 43
Solubility and viscosity data of oligosaccharides up to 200 mg/mL at 25° C.

| | Solubility at 25° C. | | | Absolute Viscosity at 25° C. (mPa · s) | | |
|---|---|---|---|---|---|---|
| | 200 mg/mL | 100 mg/mL | 50 mg/mL | 100 mg/mL | 50 mg/mL | 10 mg/mL |
| CLX114 | Soluble, | Soluble, | Soluble, | 1.877 | 1.214 | 0.903 |
| | yellow | off-white | clear | | | |
| CLX113 | Soluble, | Soluble, | Soluble, | 1.209 | 1.019 | 0.825 |
| | clear | clear | clear | | | |
| CLX112 | Soluble, | Soluble, | Soluble, | 1.248 | 1.131 | 0.649 |
| | clear | clear | clear | | | |
| CLX111 | Soluble, | Soluble, | Soluble, | 1.683 | 1.163 | 1.051 |
| | tan | off white | clear | | | |
| CLX110 | Turbid, | Soluble, | Soluble, | 1.250 | 1.087 | 0.769 |
| | off white | clear | clear | | | |
| CLX109 | Soluble, | Soluble, | Soluble, | 0.830 | 0.983 | 0.691 |
| | amber | light amber | clear | | | |
| CLX108 | Cloudy, | Cloudy, | Cloudy, | 6.447 | 2.313 | 0.819 |
| | off-white | white | white | | | |
| CLX105 | Soluble, | Soluble, | Soluble, | 1.210 | 0.982 | 0.935 |
| | yellow | yellow | off-white | | | |
| CLX103 | Cloudy, | Soluble, | Soluble, | 1.093 | 0.945 | 0.861 |
| | yellow | off-white | off-white | | | |
| CLX102 | Soluble, | Soluble, | Soluble, | 1.392 | 1.140 | 0.716 |
| | clear | clear | clear | | | |
| CLX101 | Turbid, | Cloudy, | Cloudy, | 1.306 | 1.101 | 0.712 |
| | off white | off white | white | | | |

### Example 17

### (The Use of Iron, or Copper, for Generating Oligosaccharides)

Production of Oligosaccharides. In an exemplary aspect, nine aliquots of CLX115 (300 mg) were dissolved in 6.9 ml of 40 mM Sodium acetate buffer adjusted to pH 5.51 in a capped reaction vessel and placed in a shaker-incubator for 10 min at 55° C. and 85 RPM. Hydrogen peroxide (1.71 ml) was added to the tubes. Followed by either iron (II) sulfate (1.43, 2.07, 2.86 or 5.72 mg) or copper (II) sulfate (0.107, 0.321, 0.643, 1.26 or 1.86 mg) were added to the reaction mixture and mixed thoroughly. The reactions in capped reaction vessels were allowed to proceed in the shaker-incubator at 55° C. and 65 RPM for two hours. The capped reaction was allowed to cool to 20° C. All oxidation conditions were subject to the following cleavage via the addition of 300 ul of concentrated ammonium hydroxide. The reactions in capped reaction vessels were allowed to proceed at 45° C. in a shaker-incubator for 1 hour at 70 RPM, the cap was left loose to allow oxygen, ammonia, and carbon dioxide gases to be released.

Purification. Each reaction was subjected to deionization via the addition of 3 ml of hydrated Lewatit NM 60 mixed bed resin to each reaction vessel. The mixture was allowed to stir at room temperature for 35 min then the resin was removed through course filtration. The resulting eluent was frozen and lyophilized. Once all water had been removed the pellet was rehydrated in a 70% aqueous ethanol the resulting precipitate pelted via centrifugation at 6500 rpm for 15 min. The pellet was discarded, and the supernatant was concentrated via rotary evaporation under reduced pressure.

TABLE 44
Oligosaccharide yields with varying catalyst
concentration and composition.

| Catalyst and concentration | Yield |
|---|---|
| Fe (II) sulfate 0.6 mM | 41% |
| Fe (II) sulfate 0.87 mM | 57% |
| Fe (II) sulfate 1.2 mM | 59% |
| Fe (II) sulfate 2.4 mM | 79% |
| Copper (II) sulfate 0.05 mM | 33% |
| Copper (II) sulfate 0.15 mM | 26% |
| Copper (II) sulfate 0.3 mM | 26% |
| Copper (II) sulfate 0.6 mM | 22% |
| Copper (II) sulfate 0.87 mM | 18% |

Data: Exemplary data has shown as follow in respect to the composition and the effect on metabolite production in a fecal microbiota community.

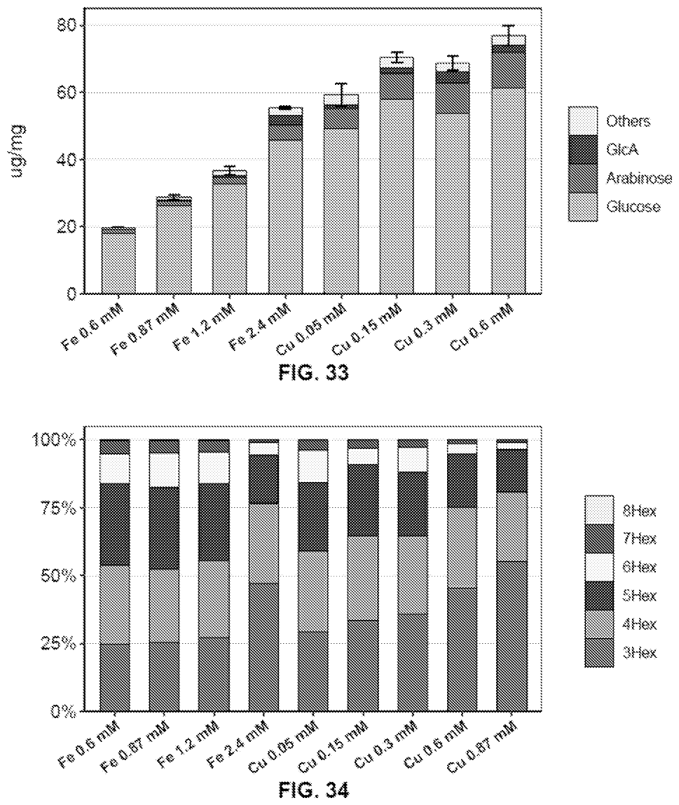

When examining the effect of both catalyst concentration and identity a few clear trends became apparent. When examining the free monosaccharide concentrations, the increase of concentration of iron leads to greater amount of free monosaccharide being produced (FIG. 33). In concert with the yield data (Table 44), this increase in inconsequential due to the increased reaction efficacy. In contrast the differences in Copper lead to very little changes in change in the total amount of free monosaccharide produced, however the reaction efficacy is depressed in comparison to Iron. When examining the oligosaccharide DP compositions of the various catalyst amounts there is little change in the iron trial apart from the highest concentrations. At 2.4 mM Iron caused an increase in the relative amounts of the smaller oligosaccharides. In contrast there is upward trend in concentrations of smaller oligosaccharides as the amount of copper catalyst is increased (FIG. 34).

### Example 18

(Selective DP Isolation of Beta Glucan Oligosaccharides and their Impact on the Microbiota)

Production of Oligosaccharides. In a first exemplary aspect, CLX115 (15 g) were dissolved in 218.2 ml of 40 mM Sodium acetate buffer adjusted to pH 5.61 in a reaction vessel and placed in a shaker-incubator for 10 min at 55° C. and 85 RPM. Hydrogen peroxide (54.6 ml) was, followed by iron (II) sulfate (56.9 mg in 500 μL water) the reaction mixture and mixed thoroughly. The reactions in capped reaction vessel proceeded in the shaker-incubator at 55° C. and 150 RPM for two hours. The capped reaction cooled to 20° C. The cleavage was initiated via the addition of 15 mL of concentrated ammonium hydroxide. The reaction vessel proceeded at 45° C. in a shaker-incubator for 1 hour at 150 RPM, the cap was left loose to allow oxygen, ammonia, and carbon dioxide gases to be released.

Purification. Each reaction was subjected to deionization via the addition of 150 ml of hydrated Lewatit NM 60 mixed bed resin to each reaction vessel. The mixture was allowed to stir at room temperature for 45 min then the resin was removed through course filtration. The resulting eluent was frozen and lyophilized.

Once all water had been removed the pellet 2 g of the material was allocated was rehydrated in 20 mL of 80% aqueous ethanol, the resulting precipitate pelted via centrifugation at 6500 rpm for 15 min. The supernatant was concentrated via rotary evaporation under reduced pressure. The 80% pellet was rehydrated in 20 mL of 60% aqueous ethanol the resulting precipitate pelted via centrifugation at 6500 rpm for 15 min. The supernatant was concentrated via rotary evaporation under reduced pressure. Finally, the 60% pellet was rehydrated in 20 mL of 40% aqueous ethanol the resulting precipitate pelted via centrifugation at 6500 rpm for 15 min. The supernatant was concentrated via rotary evaporation under reduced pressure. The resulting 40% pellet was frozen and lyophilized.

Data: Exemplary data shown herein demonstrates the impact that composition has on metabolite production in a fecal microbiota community. Ethanol was used at different concentrations to fractionate a sample into different DP ranges. We observed a direct correlation between ethanol concentration and free monosaccharide composition, with the highest free monosaccharide concentration seen in the 80% ethanol supernatant (FIG. 35). Conversely there was little difference between the concentrations of free monosaccharide for the ethanol precipitate samples due to the larger DP distribution of the materials (FIG. 36). When examining the oligosaccharide DP analysis, we observed minimal differences in the oligosaccharide analysis profile from the ethanol precipitate due to the inability to analyze large polymers with this platform, this is visualized via the low peak area seen in both EP samples where the non-observed fraction is resultant from DP 8+ material. Overall, there is a clear shift of the distribution of DP being greater in the ethanol precipitate fractions as seen in the 6hex oligosaccharides specifically. Furthermore, a distinct enrichment of the smaller DP sizes can be seen in 80% ethanol precipitate with the higher levels of DP 3 and 4.

Fractionating CLX115 with varying ethanol concentrations resulted in colloidal suspensions of oligosaccharide at room temperature up to 20% (w/w) at each ethanol concentration tested (Table 45). The greater abundance of higher length oligosaccharides (i.e., supernatants of oligosaccharides treated with a lower percentage of ethanol) resulted in higher viscosity solutions than in the supernatants with smaller average DP. This demonstrates the ability to selectively tune the rheological properties of the oligosaccharides generated by this method.

TABLE 45
Solubility and viscosity of oligosaccharides selectively
fractionated with various ethanol concentrations.

| | | | | Absolute |
|---|---|---|---|---|
| | | | | Viscosity at |
| | Solubility at 25 C. | | | 25 C. (mPas) |
| Oligosaccharide | 200 mg/mL | 100 mg/mL | 50 mg/mL | 50 mg/mL |
| CLX115 40% EtOH Supernatant | Cloudy, off- | Cloudy, off- | Cloudy, off- | 2.378 |
| | white | white | white | |
| CLX115 60% EtOH Supernatant | Cloudy, | Cloudy, tan | Cloudy, tan | 1.097 |
| | amber | | | |
| CLX115 70% EtOH Supernatant | Soluble, | Soluble, | Soluble, | 1.382 |
| | clear | clear | clear | |
| CLX115 80% EtOH Supernatant | Soluble, tan | Soluble, tan | Soluble, tan | 1.330 |
| CLX115 40% EtOH Precipitate | Insoluble | Insoluble | Insoluble | n/a |
| CLX115 70% EtOH Precipitate | Insoluble | Insoluble | Insoluble | n/a |

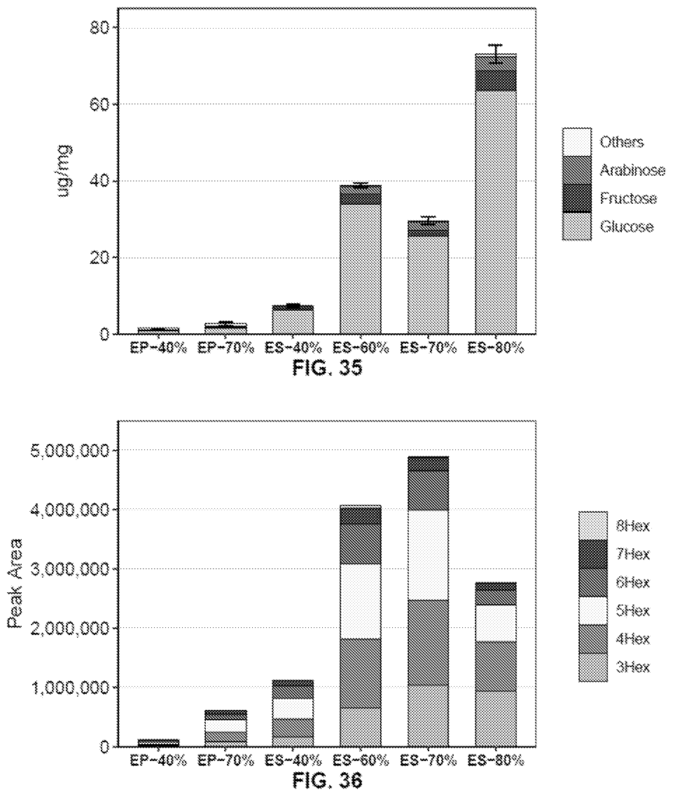

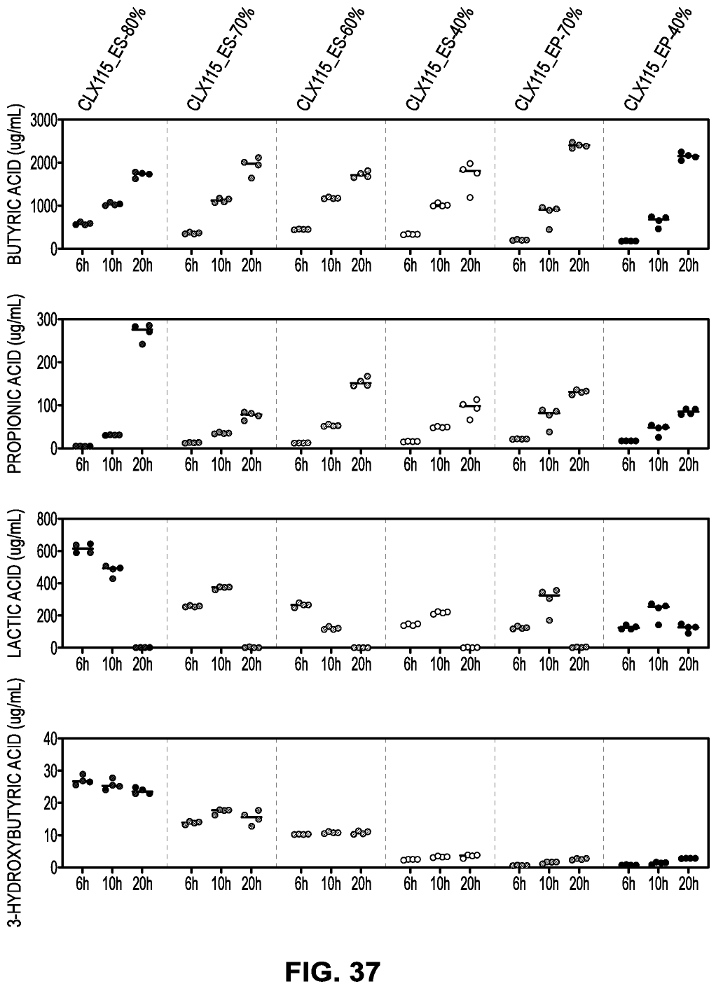

When examining the SCFA generated during the fecal microbiota fermentation, trends become apparent. As the concentration of ethanol increased there was a trend of higher levels of production of propionic, lactic and 3-hydroxybutyric acids (FIG. 37). Additionally, as the amount of ethanol decreases the propionic, lactic, and 3-hydroxybutyric acid production rate is slower. However, butyric acid production steadily increased regardless of purification scheme. Comparing the EP fractions production of 3-hydroxybutyric acid is low through the incubation time period, whereas the addition of the ethanol soluble fractions stimulates this pathway.

### Example 19

(Comparison of Similar Oligosaccharide Structures from Different Sources)

Comparison of modulation on fecal microbial community metabolite production using different sources of oligosaccharides with high concentrations of Beta linked glucose.

Data: Exemplary data has shown as follow in respect to the composition and the effect on metabolite production in a fecal microbiota community. While CLX101, CLX110, CLX112 and CLX115 all have beta linked 3-glucose and most have 4-glucose in various amounts, the linkage data reveals difference in their construction (Table 46). The overall oligosaccharides pools of CLX115 and CLX101 are composed of larger DP units on average due to the lower levels of terminal units in comparison to 3/4 linked glucose, whereas CLX110, and CLX112 both have a higher ratio of terminal to linear linkages, indicating shorter polymers. General composition between the four glucose rich oligosaccharide pools does vary with CLX115, CLX110, and CLX112 all containing more 4 linked glucose than 3 linked, apart from CLX101 which contains no 4 linked glucose. In addition, both CLX115 and CLX112 have similar ratios of 3 to 4 linked glucose with CLX110 having significantly higher levels of 4 linked glucose.

TABLE 46
Linkage Composition of different beta
glucan type oligosaccharides.

| | | | | | 100*Ratio |
|---|---|---|---|---|---|
| | | | | | of T to |
| | % of 4- | % of 3- | % of T- | % of 3 to | 3 + 4- |
| ID | Glucose | Glucose | Glucose | 4-Glucose | Glucose |
| CLX115 | 64.23 | 23.09 | 12.67 | 35.95 | 14.51 |
| CLX112 | 48.91 | 17.06 | 30.95 | 34.88 | 46.92 |
| CLX101 | 0 | 74.84 | 8.82 | N/A | 11.79 |
| CLX110 | 42.76 | 25.57 | 22.71 | 59.80 | 33.24 |

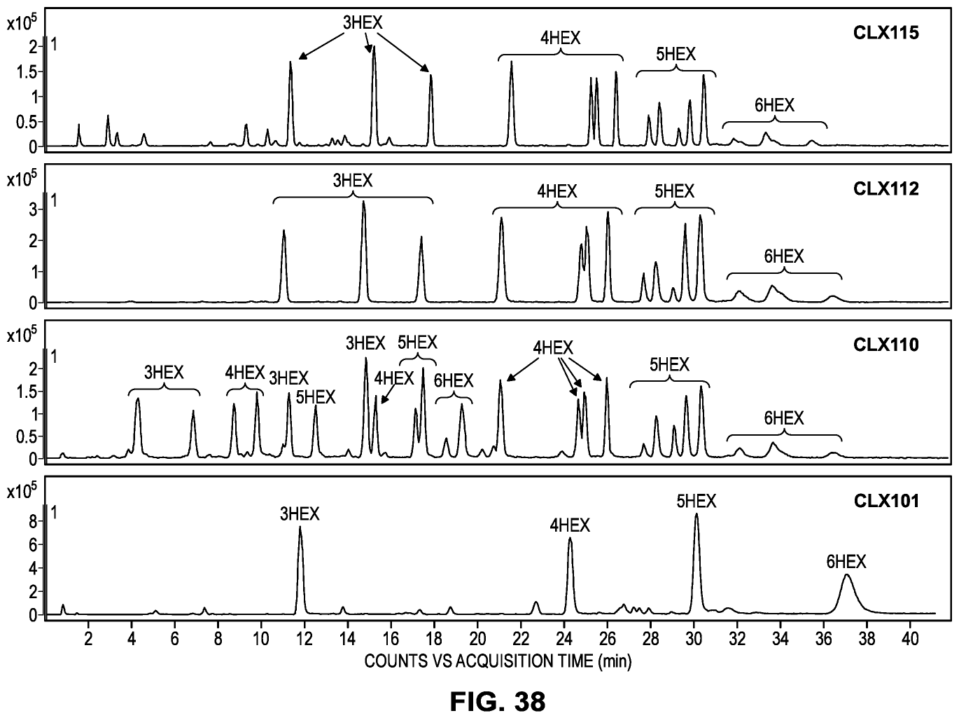

When examining the oligosaccharide composition of CLX101, CLX110, CLX112 and CLX115, the differences in each of the pool become clear and are shown in FIG. 38. Both CLX112 and CLX115 have very similar compositions and are derived from barley and oat, respectively. All peaks matched for three through six glucose units, which indicates a highly similar structure, and in turn, biological activity. While CLX110 and CLX101 differ significantly, CLX110 has many more peaks than the other samples, this increase denotes a more complicated structure with more unique isomers at each of the various DP's. The structural complexity of CLX110 is likely due to the additional mannose and galactose units observed. Inversely, CLX101 has very few peaks denoting a relatively simple structure, in concert the linkage data results this verifies a homogeneous structure comprised of only beta 3-linked bonds.

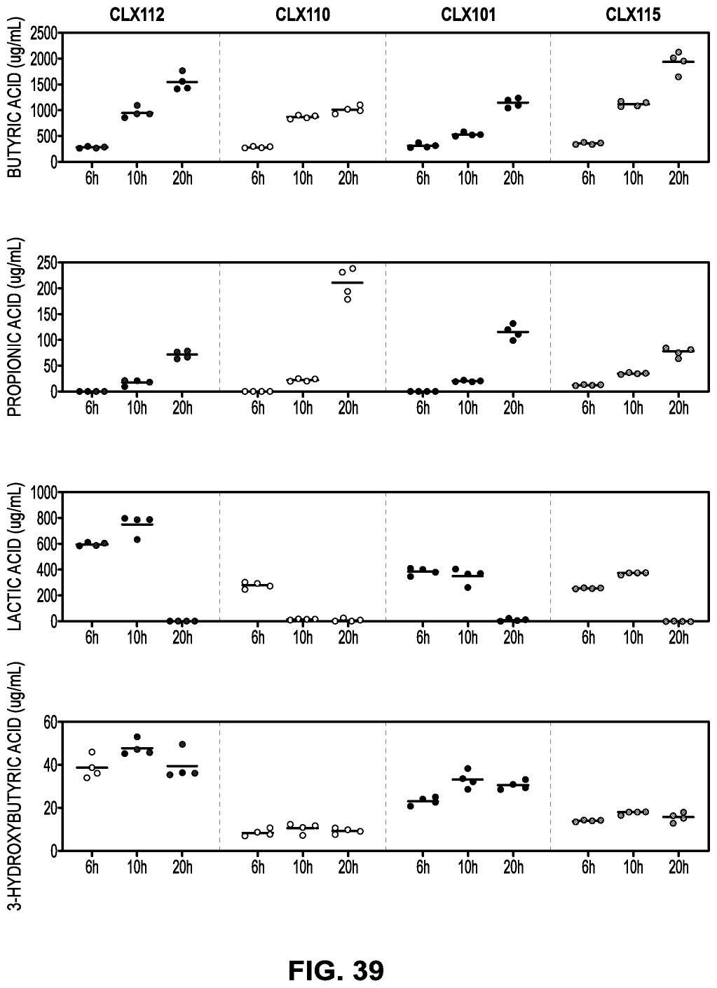

When examining the SCFA profiles of these pools after ex-vivo fecal fermentations, a clear trend become apparent (FIG. 39). We observed that all of the oligosaccharide pools will modulate the fecal community to produce butyric acid and propionic acid; However, in CLX110 lactic acid production falls after the 6-hours, in contrast to the three other oligosaccharide mixtures, which continue to produce lactic acid until at least 10-hours. Finally, CLX110 and CLX115 produced similarly low levels of 3-hydroxybutyric acid across the entire fermentation window. Conversely, CLX101 and CLX112 produced at least twice the 3-hydroxybutyric acid than the other two oligosaccharide pools.

### Example 20

### (CaCo2 Cell Model of Gastrointestinal Barrier Functioning)

Caco2 cells were cultured in high glucose Dulbecco's modified Eagle medium supplemented with 10% fetal bovine serum (FBS), 2 mM L-Gln, non-essential amino acids, 100 U/mL penicillin/100 μg/mL streptomycin (Gibco, Waltham, MA, USA), and 20 mM Hepes. The cells (passage number 14) were seeded on 13 mm polyester transwell inserts with 0.4 μm pore size (Corning, NY, USA). The cell culture medium was refreshed twice weekly. Following 20 days of Caco2 cell culture on transwells, the basolateral chamber was supplemented with 10 ng/uL tumor necrosis factor alpha (TNF-alpha) and interferon gamma (IFN-gamma) and supernatants obtained in accordance with Example 3 were diluted 1:2 in culture medium and were added to the apical side followed by incubation for 48 h. TNF-alpha and IFN-gamma were included to disrupt Caco2 barrier integrity and promote inflammation. Na-butyrate control (5 mM) and cell culture medium were used as controls.

After 24 h incubation, the medium containing the supernatants was refreshed. The integrity of the CaCo2 cell barrier integrity was then measured using transepithelial electrical resistance (TEER). This involved the measurement of electrical resistance across a cellular monolayer. Two electrodes were used, with one electrode placed in the upper compartment and the other in the lower compartment and the electrodes were separated by the cellular monolayer. An alternating current (AC) voltage signal with a square waveform was applied using an Epithelial Voltohmmeter (EVOM) at a frequency of 12.5 Hz. Three separate experiments were conducted and a minimum of three and up to six replicates for each sample were used depending on the experimental conditions.

Further, after incubation for 24 h, the apical media was harvested for cytokine analysis and protein expression. Cytokine secretion and protein expression were analyzed via mRNA expression and quantified by qPCR.

The results indicate that supernatants, obtained in Example 3, from CLX115, CLX122, CLX 114 and CLX102 significantly improved barrier integrity of Caco2 cells compared to the untreated control. Supernatants from CLX122, CLX114 and CLX102 improved barrier integrity similarly to 5 mM Na-Butyrate, a short chain fatty acid known to improve Caco2 barrier integrity and aid barrier function.

### Example 21

### (Production of Oligosaccharides)

In a first exemplary aspect, 10 g of pea fiber was dissolved in 228.6 ml of 40 mM Sodium acetate buffer adjusted to pH 5.55 in a reaction vessel and placed in a shaker-incubator for 10 min at 55° C. and 85 RPM. Hydrogen peroxide (57.1 ml) was, followed by iron (II) sulfate (59.6 mg in 500 μL water) the reaction mixture and mixed thoroughly. The reactions in a capped vessel proceeded in the shaker-incubator at 55° C. and 150 RPM for two hours. The reaction cooled to 12° C. The cleavage was initiated via the addition of 7.72 mL of concentrated ammonium hydroxide. The reaction vessel proceeded at 45° C. in a shaker-incubator for 1 hour at 150 RPM, the cap was left loose to allow oxygen, ammonia, and carbon dioxide gases to be released.

Purification. The reaction was subjected to deionization via the addition of 100 ml of hydrated Lewatit NM 60 mixed bed resin to each reaction vessel. The mixture was allowed to stir at room temperature for 45 min then the resin was removed through course filtration. The resulting eluent was frozen and lyophilized.

Once all water had been removed 2 g of the material was allocated and rehydrated in 20 mL of 70% aqueous ethanol, the resulting precipitate was pelted via centrifugation at 6500 rpm for 15 min. The supernatant was collected then concentrated via rotary evaporation under vacuum prior to lyophilization.

### Example 22

### (Production of Destarched Pea Fiber Oligosaccharides)

50 g of pea fiber was added to 600 ml of 40 mM Sodium acetate buffer adjusted to pH 5.15 in a reaction vessel to create a slurry. To the mixture 800 mg of Alpha amylase were added, the mixture was then placed in a shaker-incubator for 18 hours at 45° C. and 155 RPM. Once time had elapsed the resulting solution was centrifuged at 7500 rpm for 20 min with the supernatant being discarded. The resulting pellet was subjected to 2 iterative 200 mL 90% aqueous ethanol washes at 55° C. and 155 RPM discarding the liquid layer both time. Once complete the resulting powder was dried over a vacuum resulting in 27 grams of white powder.

Of the stock of white powder, 8 g was dissolved in 175 ml of 40 mM Sodium acetate buffer adjusted to pH 5.55 in a reaction vessel and placed in a shaker-incubator for 10 min at 55° C. and 85 RPM. Hydrogen peroxide (54 ml) was, followed by iron (II) sulfate (47.7 mg in 500 μL water) the reaction mixture and mixed thoroughly. The reactions in a capped vessel proceeded in the shaker-incubator at 55° C. and 150 RPM for two hours. The reaction cooled to 12° C. The cleavage was initiated via the addition of 6.17 mL of concentrated ammonium hydroxide. The reaction vessel proceeded at 45° C. in a shaker-incubator for 1 hour at 150 RPM, the cap was left loose to allow oxygen, ammonia, and carbon dioxide gases to be released.

Once complete the reaction was subjected to deionization via the addition of 120 ml of hydrated Lewatit NM 60 mixed bed resin to the reaction vessel. The mixture was allowed to stir at room temperature for 45 min, then the resin was removed through course filtration. The resulting eluent was frozen and lyophilized. Once all water had been removed the material was rehydrated in 20 mL of 70% aqueous ethanol, the resulting precipitate was pelted via centrifugation at 6500 rpm for 15 min. The supernatant was collected then concentrated via rotary evaporation under vacuum prior to lyophilization.

### Example 23

### (Effect of CLX 122 on Single Strain Growth)

To determine the oligosaccharide conducive to selective bifidobacteria growth, selected bacterial strains were grown in the presence of oligosaccharides as sole carbon source and assayed for growth and metabolic output. Selected bifidobacteria strains include microbes from the American Type Culture Collection and human isolates.

Pure cultures were incubated in minimal media containing oligosaccharide composition CLX 122 in a final concentration of 2% w/v. Growth was monitored by measuring OD (600 nm) using microtiter plate-reader (Epoch 2, BioTek). Specifically, fresh cultures were generated by transferring a colony (per replicate) into fresh liquid media and incubating in an anaerobic chamber (Anaerobic Chamber Vinyl Type B), using a mix of gas (carbon dioxide 5%, hydrogen 5%, nitrogen balance). De Man, Rogosa and Sharpe media (MRS) was used to grow Bifidobacterium species. Cells were harvested at late exponential phase and an experimental inoculum was prepared by washing once with phosphate-buffered saline (PBS). This was done by centrifuging the culture at 8000 g for 5 min, discarding the supernatant, and resuspending in the same volume of reduced PBS. Minimal media containing 2% of CLX 122 oligosaccharides or no carbon source (negative control) were inoculated with 2% of the experimental inoculum. Growth was run 4 times (replicates) and media sterility was tested by incubating non-inoculated media. Growth was determined based on absorbance measurements at 600 nm. Minimal media used for Bifidobacteria was basal MRS previously described by Ruiz-Moyano et al. (2013).

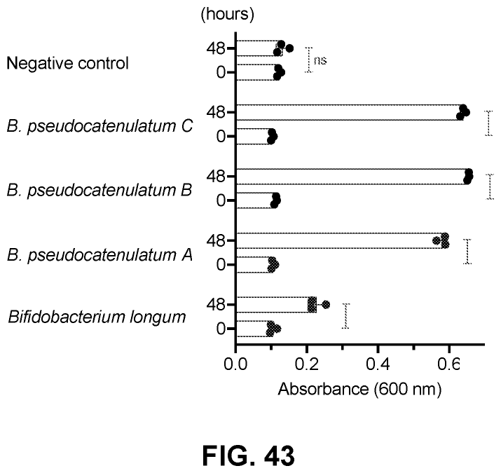

Differential growth of tested strains was observed due to the presence of CLX122 oligosaccharides used as a sole carbon source. Growth of different Bifidobacteria pseudocatenulatum and Bifidobacterium longum subsp. longum strains was supported by CLX122 (FIG. 43).

Growth of Bifodobacterium species in CLX122 was strain specific. Different growth levels were observed when CLX 122 oligosaccharides were used as a sole carbon source. Specifically, CLX 122 supported strong growth of both Bifidobacteria pseudocatenulatum and Bifidobacterium longum subsp. longum strains but not any other of the tested strains (FIG. 43).

### Example 24

### (Effect of Oligosaccharides on Ex-Vivo Human Fecal Microbiota and Associated Metabolites)

Fecal samples were collected from healthy donors by BioIVT and stored at −80 C until processing. Aliquots of slurry from the fecal samples were prepared mixing three parts of fecal samples, one part of glycerol and one part of PBS. Slurries were stored at −80 C. Static fecal fermentations were conducted in a deep 96-well format, under anaerobic conditions (Anaerobic Chamber Vinyl Type B), using a mix of gas (carbon dioxide 5%, hydrogen 5%, nitrogen balance), using a pool of feces from 8 individual donors at a final concentration of 2% of the fermentation mix.

CLX122, CLX128, CLX127 and CLX126 oligosaccharide compositions were tested at a concentration of 0.6% w/v. Fermentation media was optimized to support diverse microbial taxa and control pH within the range of the colon physiological conditions, containing mineral and vitamin solution, CaCl₂ (10 mg/ml) and basic fermentation medium as described by MacFarlane G T et al (1989). A mix of background sugars (xylan, amylopectin, potato starch, and pectin) were included in the basic fermentation media in low concentration to sustain microbial networks and minimize changes due to lack of nutrients which would confound the experimental results. Multiple samples were taken at different time points (0 h, 6 h, 11 h and 20 h) for monosaccharide analysis, and SCFA analysis. After 20 hours of fermentation, genomic DNA was extracted from each well and sent for 16S rRNA sequencing. Four replicates were run for each oligo and untreated control.

### Microbiota Analysis

DNA was extracted from fecal slurry using ZymoBIOMICS Kit D4308 and a KingFisher Flex DNA extraction robot. Microbial communities were profiled by sequencing the V4 region of the bacterial 16S rRNA gene amplified using 515F (5′ GTGCCAGCMGCCGCGGTAA 3′) and 806R (5′ GGACTACHVGGGTWTCTAAT 3′) primers. NovaSeq 6000 was used to obtain 250 bp paired end reads. Raw demultiplexed reads were processed using QIIME2 2020.11. Briefly, after quality checking, trimming, filtering and denoising were performed using the “dada2 denoise-paired” plugin in QIIME2. Taxonomic classification of ASVs was performed with the “q2-feature-classifier” plugin and a Naive Bayes classifier trained on Silva 138 99% OTUs from the 515F/806R region of 16S rRNA sequences. Finally, beta diversity analyses were conducted with QIIME2 plugins and subsequently imported into MATLAB, R and Excel for further analysis.

CLX 122 has a different impact in the microbial composition of the fecal communities compared to untreated samples (absence of oligosaccharide in the fecal fermentation medium) at phylum and species level. We evaluated the effect of the oligosaccharides in different taxa and species of bacteria with potential impact on host health (Table 47).

TABLE 47
Changes in relative abundance of microbes in fecal fermentations
with tested oligos versus untreated control, with a pool of
8 fecal samples. Oligosaccharides identified by CLX ID.

| | CLX | CLX | CLX | CLX |
|---|---|---|---|---|
| Taxonomic group | 122 | 126 | 127 | 128 |
| Actinobacteria | + | + | + | + |
| Bifidobacterium | + | + | + | + |
| Bacteroides | + | − | + | − |
| Bacteroides_intestinalis | − | − | − | − |
| Bacteroides_ovatus | + | + | 0 | 0 |
| Parabacteroides_distasonis | − | − | − | − |
| Firmicutes | + | + | − | + |
| Lactobacillus | 0 | 0 | + | + |
| Clostridia | + | − | − | + |
| Clostridiaceae | + | − | − | − |
| Clostridium_butyricum | + | + | − | + |
| Lachnospiraceae | + | + | + | + |
| Ruminococcus gnavus | + | + | − | + |
| Ruminococcus torques | − | + | + | + |
| Blautia | + | + | + | + |
| Roseburia | + | + | + | + |
| Faecalibacterium | + | + | + | + |
| Ruminococcus | 0 | 0 | + | + |
| Proteobacteria | − | − | − | − |
| Akkermansia | − | − | − | − |

Key symbols: + = increase in relative abundance vs untreated control; − = decrease in relative abundance vs untreated control; 0 = no difference.

Bifidobacterium, a well-known genus associated with beneficial health effects and positive impacts on gut mucosal barrier, was increased by CLX 122, CLX 128, CLX 127 and CLX 126. Most of the increase was associated to the species Bifidobacterium pseudocatenulatum, whose relative abundance was significantly higher in the presence of the mentioned substrates compared to the untreated control. (FIG. 44).

The genus Bacteroides, a major commensal within the human gut and a mucin and plant fiber consumer was enriched for two out of the four oligosaccharide compositions tested. These are CLX 122 and CLX 127 (FIG. 45).

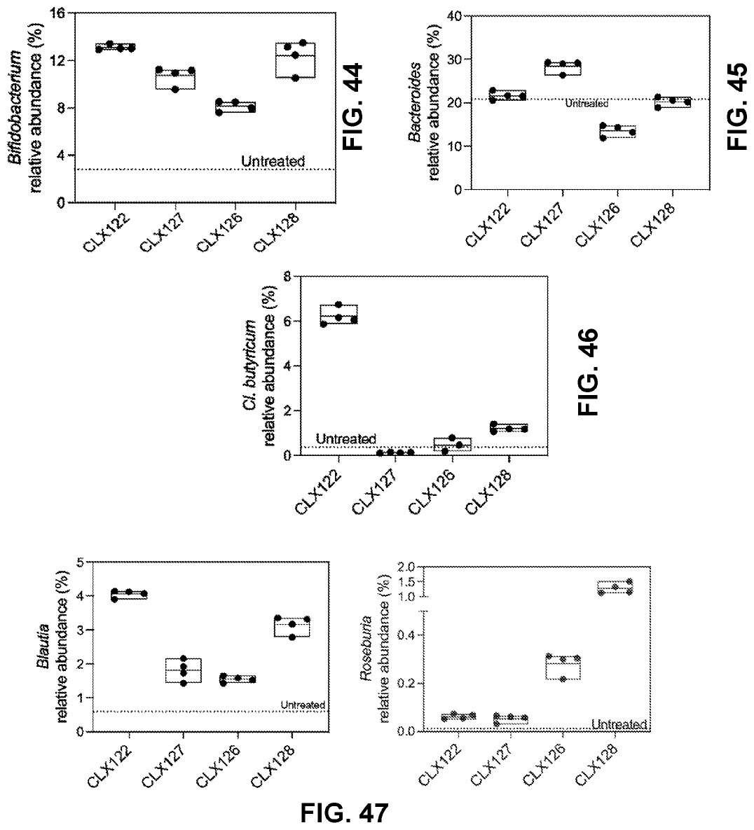

Firmicutes were enriched by CLX 122, CLX 126 and CLX 128 but not for CLX 127. Compared to all of the tested oligosaccharide compositions, CLX 122 enriched Cl. butyricum the most compared to the untreated control (FIG. 46). Ruminococcus genus was enriched when CLX 127 and CLX 128 was employed. Ruminococcus gnavus group was enriched in the presence of CLX 122, CLX 126 and CLX 128, while Ruminococcus torques group was detected at higher levels in CLX 126, CLX 127, CLX 128 fermentations. Blautia and Roseburia were enriched in all four tested oligosaccharide compositions (FIG. 47). All the tested oligosaccharide compositions decreased Proteobacteria phylum compared to the untreated control.

### Short Chain Fatty Acid (SCFA) Analysis

Supernatants of fecal bacterial growths were derivatized with 2-nitrophenylhydrazine (2-NPH) for LC-MS/QqQ analysis. In brief, supernatants of fecal bacterial growths were diluted with water (1:20). Twenty microliters of the supernatant dilutions were added to 20 μL of N-(3-Dimethylaminopropyl)-N′-ethylcarbodiimide hydrochloride (1-EDC HCl) in 5% pyridine. Then 40 μL of 200 mM 2-NPH in 80% acetonitrile with 50 mM HCl was added and briefly vortexed prior to incubating for 30 minutes at 40° C. After incubation, samples were diluted with 400 μL of 10% acetonitrile and vortexed. Aliquots of the samples were transferred to 96 well plates for LC-MS/QqQ analysis.

Short chain fatty acids (SCFAs) were analyzed with a 1290 Infinity II LC (Agilent Technologies, Santa Clara, CA) equipped with a reverse phase column (Zorbax Eclipse C18 2.1×50 mm; Agilent Technologies, Santa Clara, CA) and 6490 Triple Quad LC/MS (Agilent Technologies, Santa Clara, CA). LC separation was performed with 5% acetonitrile with 0.1% formic acid (solvent A) and 95% acetonitrile with 0.1% formic acid (solvent B). A separation gradient was as follows: 5% to 20% B for 2 minutes, then 20% to 40% B for 1 minute, 40-55% B in 1 minute, 55-100% B in 0.1 minute, hold at 100% B for 0.5 minutes, return to 5% B in 0.1 minute, and equilibrate at 5% B for 0.99 minutes. ESI-MS conditions were performed in positive mode, and the dynamic multiple reaction monitoring (dMRM) mode was used to monitor the precursor and product ion transitions. Peak areas were quantitated using Agilent Quantitative Analysis software and areas were normalized to internal standards and compared to an external standard curve for quantitation.

Microbial short chain and organic acids were monitored during the fecal fermentation at time points 0-hour, 6-hour, 10-hour, and 20-hour and were compared to a sample containing only background sugars. Short chain and organic acids make up the key intermediate and end products of carbohydrate fermentation, many of which are known to be beneficial for human health. Butyrate is an important metabolite linked to many gut-derived diseases and is a key modulator of the immune system. However, not all carbohydrates reach their end-product metabolites and those that do, do not always reach their final state in the same way. Butyrate, for example, can be produced through different pathways that may or may not have lactic acid as an intermediate. Lactic acid production may indicate differential metabolism within an organism due to the carbohydrate source or enrichment in certain communities that employ this cycle. Bifidobacteria are known lactic acid producers while, Blautia and Clostridium are known butyrate producers.

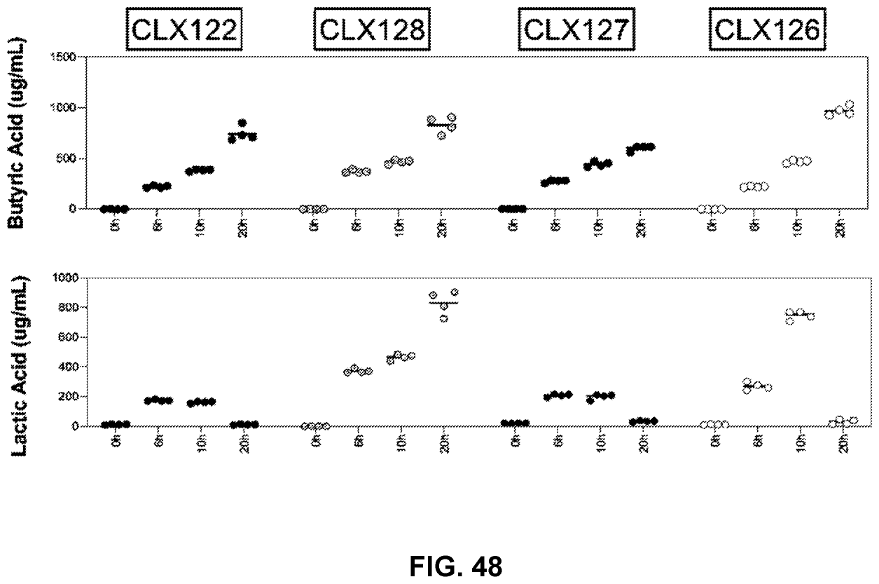

All four samples, CLX 122, CLX 128, CLX 127, and CLX 126, stimulated production of butyric acid in high amounts; however, lactic acid profiles were vastly different (FIG. 48). CLX 122 and CLX 127 produced lower amounts of lactate, which dissipated by 20-hours, which may be indicative of lactic acid being produced then converted to butyric acid. CLX 128 showed a steady increase in lactic acid, which may a different microbiota, which independently produces both butyrate and lactate. CLX 126 showed a large increase over 10 hours prior to a steep drop.

### Carbohydrate Consumption Analysis

The consumption of the compositions by the bacterial consortia in the pooled donor fecal samples were studied by analyzing and comparing the carbohydrate composition of the fermentation supernatant at 0-hours and 20-hours. Aliquoted samples were analyzed by hydrolytic monosaccharide compositional analysis. Briefly, the hydrolysis reaction to produce monosaccharides was performed at the optimized condition of 100° C. for 2 hours. Samples were run on an Agilent 1290 Infinity II ultra-high performance liquid chromatography (UHPLC) system couple to an Agilent 6490A triple quadrupole (QqQ) mass spectrometer. Separation was carried out on an Agilent InfinityLab Poroshell HPH-C18 column (2.1 mm×50 mm, 1.9 μm particle size) plus a guard column (5 mm) with the same solvent system described in the paper. With a constant flow rate of 1.2 mL/min, an isocratic gradient of 8.5% B was used for the first 4-min elution period, followed by 15% B for 0.4 min. For the flush period, 97% B was held for 1 min. The column thermostat was set at 35° C. For the mass spectrometry parameters, the only change from the method described in the paper is that the fragmentor voltage was set at 380V.For data analysis, the hydrolysis correction factor was not applied since the samples herein contains oligosaccharides instead of polysaccharides. In this analysis method, monosaccharide composition is calculated by quantifying the concentrations of 14 monosaccharides (glucose, galactose, fructose, xylose, arabinose, fucose, rhamnose, glucuronic acid, galacturonic acid, N-acetylglucosamine, N-acetylgalactosamine, mannose, allose, ribose) against their individual standard curves.

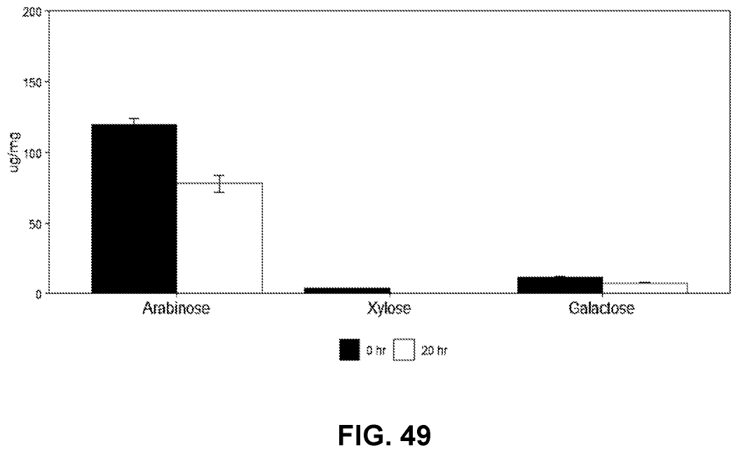

As described in the CLX 122 definition, the composition is comprised primarily of arabinose and galactose, as determined by hydrolytic monosaccharide composition analysis. Upon fermentation by the fecal fermentation donor pool, it was observed that arabinose and galactose were both consumed. Surprisingly, both arabinose and galactose were consumed at similar rates, 35.18% and 35.59%, respectively. Equal rates of consumption indicate that the combination of both arabinose, galactose, and their related glycosidic linkages are important for the mechanism of action (FIG. 49). The consumption of the composition indicates that the bloom of the Bifidobacterium species is dependent upon the fecal pools ability to degrade and consume the specific and unique composition of monosaccharides, glycosidic linkages, and oligosaccharide components that defines CLX 122 and similar compositions.

### Example 25

### (Effect of CLX 122 on Ex-Vivo Human Fecal Microbiota)

Fecal fermentations were repeated with 15 samples from individual donors, to confirm the bifidogenic effect of CLX 122. Preparation of fecal slurry, fecal fermentation assay, DNA extractions and sequencing, as well as SCFA analysis were run as described in Example 24.

### Microbiota Analysis:

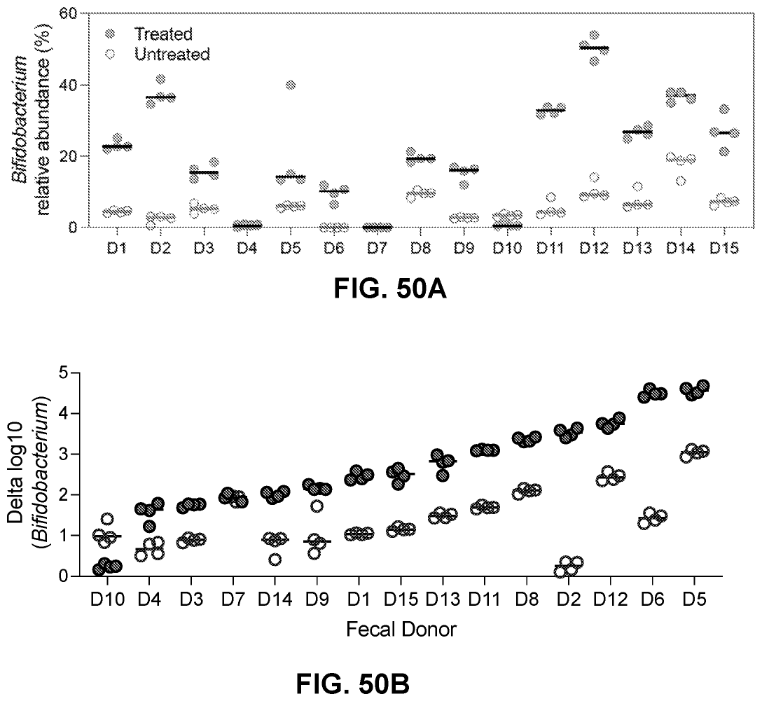

It was confirmed that CLX122 had a robust bifidogenic effect among several different complex microbial communities. Regardless of the unique microbial community composition and ecological interaction of the starting fecal communities, a significant increase in both the relative abundance and absolute abundance of Bifidobacterium was observed in 13 of the 15 fecal samples (Table 48, FIG. 50A-B). In addition, as was observed for pool 1, it was confirmed that CLX122 can significantly increase relative amount of Clostridium butyricum, Blautia and Roseburia in more than half of the samples (Table 48). A less generalized response between individuals, but still significant for some was the increase in Bacteroides, with the predominant drivers being Bacteroides vulgatus, which was enriched in 6 fecal samples, Prevotella, which was enriched in 4 fecal samples, and Parabacteroides, which was enriched in 3 out of 15 fecal samples. Lactobacillus, Faecalibacterium, or Ruminococcus were also enriched in some of the individual donor samples as shown in Table 48. In addition, CLX122 increased relative levels of Akkermansia in 6 out of the 15 donor samples.

TABLE 48
Changes in relative abundance of microbes in oligo-treated samples versus
untreated control, in 15 fecal samples from individual donors.

| Taxonomic group | D1 | D2 | D3 | D4 | D5 | D6 | D7 | D8 | D9 | D10 | D11 | D12 | D13 | D14 | D15 |
|---|---|---|---|---|---|---|---|---|---|---|---|---|---|---|---|
| Bifidobacterium | + | + | + | − | + | + | + | + | + | − | + | + | + | + | + |
| Bacteroides | − | − | − | + | + | + | + | + | + | + | − | − | − | − | − |
| Bacteroides | − | + | − | − | + | − | − | + | + | + | − | + | − | − | − |
| vulgatus | | | | | | | | | | | | | | | |
| Prevotella | 0 | − | 0 | + | + | − | + | − | 0 | 0 | 0 | 0 | 0 | + | − |
| Parabacteroides | − | − | − | − | − | − | − | − | + | − | − | + | − | − | + |
| Lactobacillus | − | + | − | − | + | + | 0 | − | − | + | + | − | + | − | − |
| Clostridium | + | − | − | + | + | + | 0 | + | + | + | + | − | − | − | + |
| butyricum | | | | | | | | | | | | | | | |
| Blautia | − | − | + | + | + | + | + | + | + | + | − | − | + | + | − |
| Roseburia | + | + | − | + | − | − | + | − | + | − | + | + | + | − | + |
| Faecalibacterium | − | + | − | + | − | + | 0 | − | − | + | − | − | − | + | − |
| Ruminococcus | + | − | − | + | − | + | 0 | − | − | + | − | − | − | − | 0 |
| Akkermansia | − | + | − | − | − | − | − | − | + | − | + | + | + | − | + |

Key symbols:
+ = significant increase in relative abundance vs untreated control; − significant decrease in relative abundance vs untreated control; 0 = no significant difference.

### Short Chain Fatty Acid Analysis

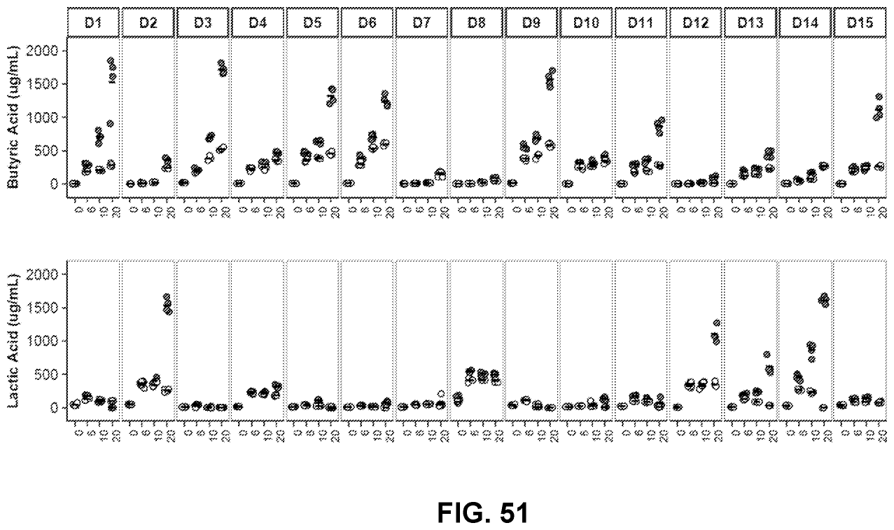

The short chain fatty acid profiles of the single donor samples were analyzed to assess the bioconversion of the CLX 122 composition into short chain fatty acid intermediate and end products. Shorty chain fatty acids, either butyrate or lactate or both, increased in all samples that also increased in Bifidobacterium relative abundance (FIG. 51). Short chain fatty acids did not increase or decreased in the two samples, D4 and D10, that did not increase in Bifidobacterium relative abundance. Short chain fatty acid profiles differed, likely due to different initial microbial populations in each donor sample and are observed in several different patterns. Samples D1, D3, D5, D6, D9, and D15, for example, had a large increase in butyrate levels but relatively little lactate production; this phenomenon is indicative of a metabolic process where lactic acid is utilized and converted to butyric acid by some types of bacteria. Samples D2, D12, D13, D14 had a large increase in lactate and relatively little increase in butyrate; this phenomenon is indicative of a metabolic process where lactic acid is produced but not converted to butyric acid due to the absence of some types of bacteria.

### Example 26

### (Effect of Additional Oligosaccharides on Ex-Vivo Human Fecal Microbiota and Associated Metabolites)

Fecal fermentations were performed as described in Example 25. CLX115A, CLX117, CLX118, CLX123, CLX124, CLX125, CLX 130, CLX131 were tested at a concentration of 0.6% w/v. Four replicates were run for each oligo and untreated control. Multiple samples were taken at different time points (0 h, 6 h, 11 h and 20 h) for monosaccharide analysis, and SCFA analysis. After 20 hours of fermentation, genomic DNA was extracted from each well, sequenced and analyzed as described in Example 4. Each oligosaccharide has a different impact in the microbial composition of the fecal community, compared to untreated sample at phylum and specie level. We evaluated the effect of the oligos in different taxa and species of bacteria with potential impact on host health, as it is shown in Table 49.

Bifidobacterium, a well-known genus associated with beneficial health effects and positive impacts on gut mucosal barrier, was increased by CLX117, CLX124 and CLX131. Most of the increase was associated to the species. Interestingly, the relative abundance of the species Bifidobacterium animalis, was increased by CLX115A and CLX118 when compared to untreated control. The genus Bacteroides, a major commensal within the human gut and a mucin and plant fiber consumer was enriched by 3 out of the oligo pools tested. These are CLX115A, CLX117 and CLX123. The species B. intestinalis increases in relative abundance in the presence of CLX125, B. ovatus with CLX129, and Parabacteroides distasonis with CLX123, CLX124 and CLX125.

Firmicutes were enriched by CLX117, CLX118, CLX123, CLX124, CLX125 and CLX129. CLX115A and CLX118 enriched Lactobacillus. Relative abundance of Clostridium increases with CLX117, CLX123, CLX124. These oligos as well as CLX125, CLX129 and CLX30 enrich Cl. butyricum species. Ruminococcus gnavus group was enriched in the presence of CLX1117, CLX130 and CLX131, while Ruminococcus torques group was detected higher levels in CLX117, CLX118, CLX123, CLX124, CLX125 and CLX131 fermentations. Blautia and Roseburia were enriched in all tested oligo pools except for CLX115A and CLX118. Faecalibacteria taxa was enriched with CLX117, CLX123, CLX125 and CLX130, while Arkkemansia increased in the presence of CLX115A, CLX125, CLX129 and CLX130.

TABLE 49
Changes in relative abundance of microbes in fecal fermentations with tested oligos versus
untreated control, with a pool of 8 fecal samples. Oligos identified by CLX ID.

| | CLX | CLX | CLX | CLX | CLX | CLX | CLX | CLX | CLX |
|---|---|---|---|---|---|---|---|---|---|
| | 131 | 130 | 125 | 129 | 124 | 123 | 117 | 115 | 118 |
| Actinobacteria | + | − | − | − | + | − | + | − | − |
| Bifidobacterium | + | − | − | − | + | − | + | − | − |
| Bifodobacterium animalis | − | − | − | − | − | − | − | + | + |
| Bacteroides | − | − | − | − | − | + | + | + | − |
| Bacteroides intestinalis | − | − | + | − | − | − | − | − | − |
| Bacteroides ovatus | 0 | 0 | 0 | + | 0 | 0 | 0 | 0 | 0 |
| Parabacteroides distasonis | − | − | + | − | + | + | − | − | − |
| Firmicutes | − | − | + | + | + | + | + | − | + |
| Lactobacillus | 0 | 0 | 0 | 0 | 0 | 0 | 0 | + | + |
| Clostridia | − | − | − | − | + | + | + | − | − |
| Clostridium butyricum | − | + | + | + | + | + | + | − | − |
| Ruminococcus gnavus | + | + | − | + | − | − | + | − | − |
| Ruminococcus torques | + | − | + | − | + | + | + | − | + |
| Blautia | + | + | + | + | + | + | + | − | − |
| Roseburia | + | + | + | + | + | + | + | − | − |
| Faecalibacteria | 0 | + | + | 0 | 0 | + | + | 0 | 0 |
| Akkermansia | − | + | + | + | − | − | − | + | − |

Key symbols:
+ = increase in relative abundance vs untreated control; − decrease in relative abundance vs untreated control; 0 = no difference.

### Example 27

### (Effect of Oligosaccharides on Probiotic Growth)

To determine the oligosaccharide that suits the best probiotic growth, four probiotic strains were grown in the presence of oligosaccharides as a sole carbon source and assayed for metabolic output. The strains belonged to the species Clostridium butyricum, Bifidobacterium pseudocatenatum, Bifidobacterium longum subsp. infantis and Bifidobacterium longum subsp. longum.

Selected strains were incubated in minimal media containing the following oligos in a final concentration of 2% w/v: CLX122, CLX124, CLX126, CLX127 and CLX128. Growth was monitored by measuring GD (600 nm) using microtiter plate-reader (Epoch 2, BioTek). Specifically, fresh culture was generated by transferring a colony (per replicate) into fresh liquid media and incubating in an anaerobic chamber (Anaerobic Chamber Vinyl Type B), using a mix of gas (carbon dioxide 5%, hydrogen 5%, nitrogen balance). Reinforced Clostridium Media (RCM) was used to propagate the Cl. butyricum strain, while MRS media was used for Bifidobacteria strains. Cells were harvested at late exponential phase and an experimental inoculum was prepared by washing once with PBS. This was done by centrifuging the culture at 8000 g for 5 min, discarding the supernatant, resuspending in PBS and precipitating the cells once more before resuspending cells again fresh basal (no carbohydrate) media.

Minimal media containing 2% of each of the tested oligos, 2% glucose or lactose (positive control) or no carbon source (negative control) were inoculated with 2% of the experimental inoculum. Growth in each media type was run 4 times (replicates) and media sterility was tested by incubating non-inoculated media. Growth was determined based on absorbance measurements at 600 nm. Minimal media used was RCM media for Cl. butyricum and MRS media for Bifidobacteria, in which we replaced glucose with the tested oligo. Measurements were exported using Gen5 2.0 software. Raw data from single strain growth was processed using an open-source tool designed to generate quantitative bacterial growth. Measurements were normalized by calculating the delta of OD₆₀₀ with a specific oligo vs negative control (media without carbohydrate), divided by the delta of OD₆₀₀ with the oligo vs positive control (2% glucose or lactose) and multiplied by 100.

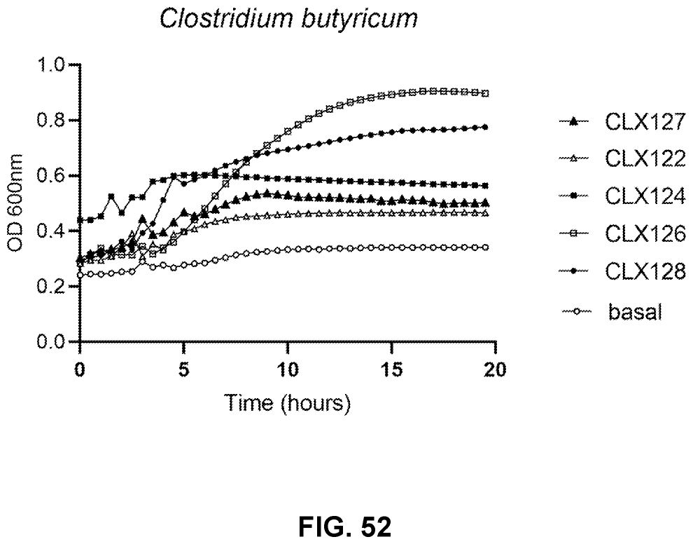

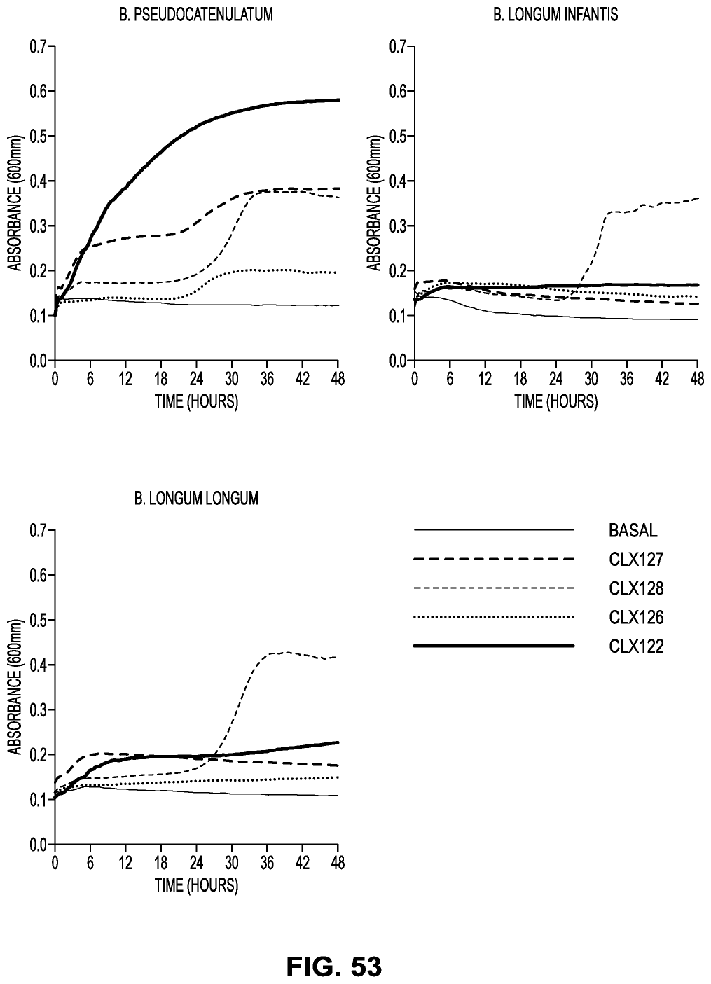

Differential growth of tested strains was observed due to the oligo used as a sole carbon source, and the number of strains they supported varies as a function of the oligo. Cl. butyricum growth was supported by CLX126 and CLX128 (FIG. 52). Bif pseudocatenatum growth was supported by CLX122, CLX126, CLX127 and CLX128. Bif longum subsp. infantis growth was supported by CLX128. Bif. longum subsp. longum growth was supported by CLX128 (FIG. 53).

These results indicate that, while many oligosaccharides can support the growth of certain bacterial strains, they each uniquely modulate the metabolic (e.g., SCFA) outputs of that bacteria. This allows the optimization of certain symbiotic pairings that are focused on metabolite modulation.

### Example 28

### (Effect of CLX 115 on In-Vivo Murine Microbiota)

The effect of CLX115 on microbial community composition and metabolite profile was evaluated in an in vivo murine model. For this purpose, 2 groups (n=8/group) of C57BL/6 mice were fed with a carbohydrate deficient diet and one of them supplemented with CLX115 (8% w/v) in the drinking water for 8 days. The second group received just water and served as a control.

### In-Vivo Murine Model of a Carbohydrate Deficient Diet

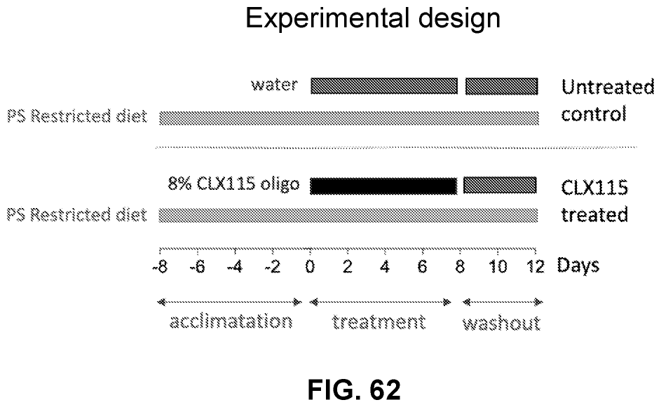

C57BL/6 mice (8 weeks old) were used to evaluate the effect of CLX115 oligosaccharides supplementation on a carbohydrate deficient murine model. Mice were divided in 6 groups (n=2-3) and acclimated for a week (7 days) while fed with a carbohydrate deficient diet (AIN-93G-Modified, Product #S6185, Bio-Serv, Flemington, NJ). At day zero of the experiment fecal samples were collected and cages divided in 2 groups. One of the groups received CLX115 in the drinking water (8% w/v) during 8 consecutive days, while the second group received just drinking water during this time. On day 9, both groups were switched back to drinking water and maintained in these conditions for an additional 4 days. This period served as a washout period to evaluate whether the effect of CLX115 is reversible (FIG. 62). Subsequently, the animals were euthanized, fecal samples were collected and serum, urine, cecal contents and tissue were sampled. Water consumption and weight were monitored throughout the experiment. Fecal samples were used to monitor the changes in microbial communities and metabolite profiles through the entire duration of the experiment.

DNA was extracted from fecal slurry using ZymoBIOMICS Kit D4308 and a KingFisher Flex DNA extraction robot. Microbial communities were profiled by sequencing the V4 region of the bacterial 16S rRNA gene amplified using 515F (5′ GTGCCAGCMGCCGCGGTAA 3′) and 806R (5′ GGACTACHVGGGTWTCTAAT 3′) primers. NovaSeq 6000 was used to obtain 250 bp paired end reads. Raw demultiplexed reads were processed using QIIME2 2020.11 as described in Example 24.

SCFA, and metabolomics assays were run as described in Example 13.

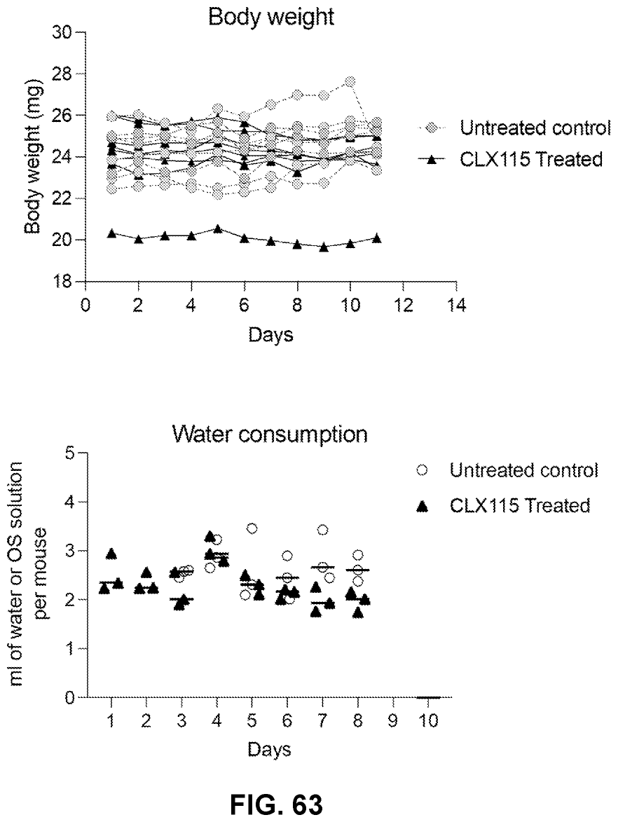

Water consumption and body weight remained constant during the study and was equivalent for both treatment groups (FIG. 63). There was one outlier mouse with a lower body weight than the rest of the cohort, but this was unrelated to CLX115 supplementation since it was underweighted from the beginning of the study. Mice remained healthy throughout the study. These results demonstrate that CLX115 is palatable to mice and had no impact on body weight or overall health status.

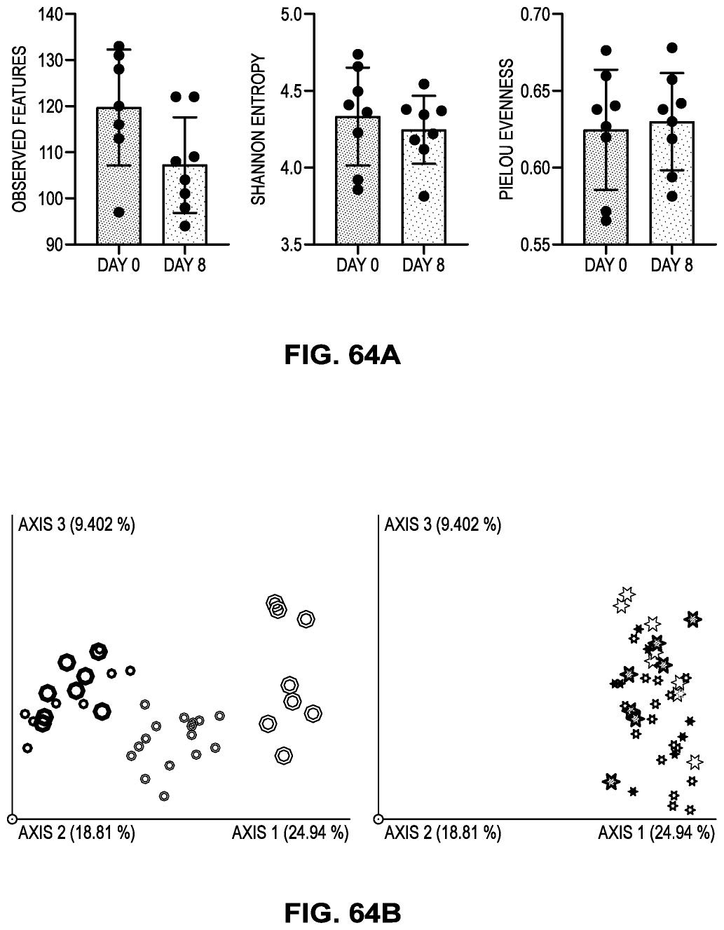

CLX115 had no effect on the alpha-diversity (observed features and/or evenness) of the murine gut microbial communities. This was determined by comparing alpha diversity scores of the communities before receiving CLX115 (day 0) and during the last day of consumption (day 8). This holds true for several different alpha-diversity metrics: observed features, Shannon entropy, and Pielou evenness (FIG. 64A). The effect of the oligosaccharide intake became evident when examining the overall microbial community membership. Dissimilarity measurements (Bray Curtis as a beta-diversity metric) and ordination methods (PCoA) revealed that communities of mice receiving CLX115 (day 6 and 8) were drastically different from the same communities before receiving the treatment (day0). However, by the last day of wash out these communities were similar to the communities at baseline (day 0), demonstrating that the oligosaccharide effect is reversible. In contrast, the fecal microbial community composition of mice in the control group was very stable throughout the study (FIG. 64B).

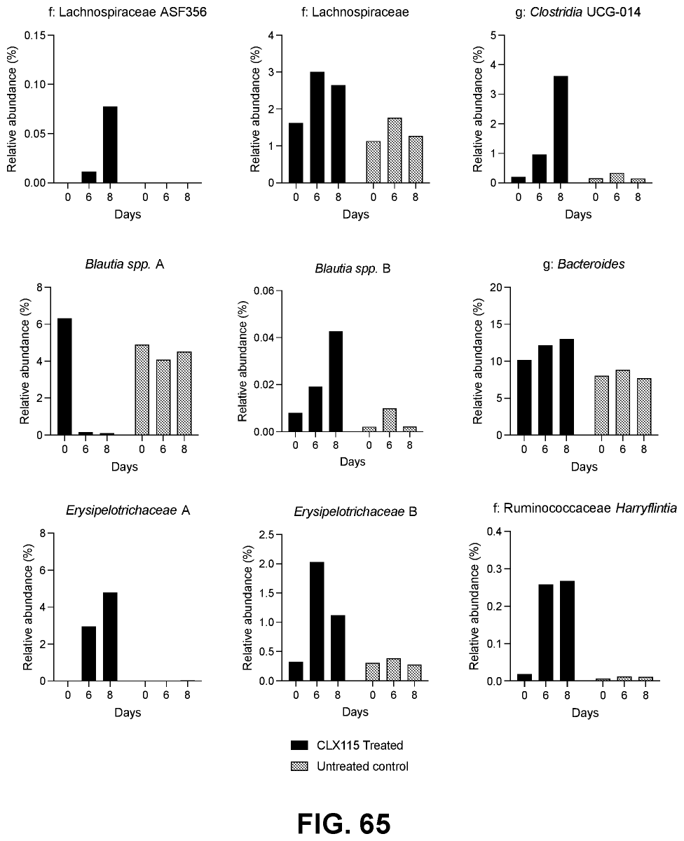

Communities' dissimilarities between day 0 and the last days of CLX115 supplementation (days 6 and 8), as well as differences between the untreated control mice and CLX115 treated mice at day 8 are reflected by enrichment of some the genus Bacteroides, some taxa within the Clostridia, Ruminococcaeae, Lachnospiraceae and Oscillospiraceae families, including some Lachnospiraceae spp., Clostridia spp., Harryflintia and other butyrate producer organisms (FIG. 65). Some species of the Blautia genus follow the same trend of being enriched by the last day of CLX115 consumption while others were depleted by the treatment. Furthermore, towards the last day of CLX115 consumption, an increase of members within the Erysipelatoclostridaceae family was observed. Interestingly taxa belonging to this same family were enriched in the human in-vitro fecal fermentation conducted using this same oligosaccharide.

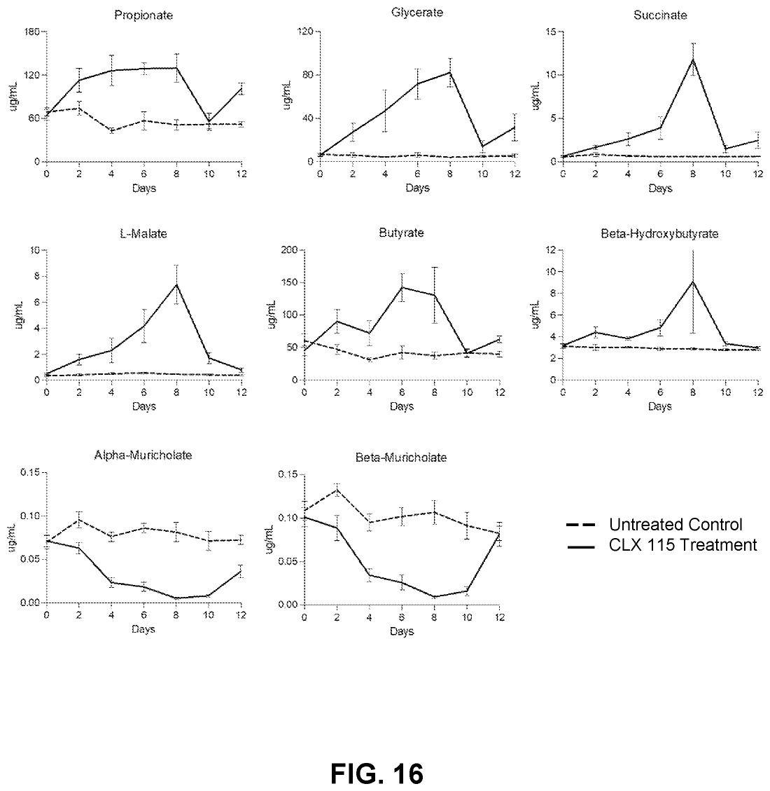

CLX115 consumption also influenced the metabolites detected in the fecal samples during the study. As expected, metabolic profiles of mice within the untreated control were very stable throughout the study. In contrast, in the group receiving CLX115 there was a steady increase in Propionate, L-Malate, Succinate, and Glycerate, since the first day of supplementation reaching the highest levels at day 8 and quickly dropping after treatment was stopped. Other metabolites such lactate, butyrate and Beta-Hydroxybutyrate showed a strong increase only during days 6 and 8. Interestingly, the spike in Butyrate and Beta-Hydroxybutyrate was significantly higher in one of the cages receiving CLX115. Even though all cages receiving the treatment had higher concentrations of these two components when compared to the control group, Butyrate and Beta-Hydroxybutyrate were 2 times higher in cage 4 that the other 2 cages. The Butyrate and Beta-hydroxybutyrate results align with the results found in the ex-vivo fecal fermentation model described in Example 13. An increase in bile acid metabolism was induced by CLX115 consumption based on a strong depletion of α-muricholate, beta-muricholate and multiple other forms of muriocholate (mice primary bile acid). These results (FIG. 16) demonstrate that CLX115 has a strong prebiotic effect, modulating both taxonomic membership and metabolic profile. Additionally, positive effects occur almost immediately after consumption starts and are quickly reversible after supplementation is interrupted.

The changes in metabolic profiles are linked with the changes in fecal microbial composition. Correlations between taxonomic and metabolic features, including short chain fatty acids (SFCA), show that enriched taxa within Lachnospiraceae and Oscillospiraceae families (such as Lachnospiraceae sp. and Harryflintia) are both potential butyrate producers given the positive correlation with butyrate levels. Additionally these taxa are positively correlated with concentrations of multiple SCFAs including, Lactate, Isobutyrate, L.Malate, Succinate, Valerate, and Pyruvate. Other positive correlations were observed between Akkermansiaceae and L Malate, members in the Clostridia genus and Succinate. Propionate was positive correlated with Blautia and lactobacillus. propionate.

Short chain fatty acids produced by microbes modulate chronic inflammation through alteration of intestinal barrier permeability and influence reverse cholesterol transport, also improving cardiovascular risk factors. Increases in SCFA production and SCFA producers have been associated with a decrease in plasma cholesterol and increase in fecal excretion of bile acids, promoting the hepatic uptake of cholesterol from the blood (Chambers E. S et al. 2018). Bile Acid modification by the gut microbiota has been suggested to have a protective effect against colonization by pathogenic organisms (Mullish B. H. et al., 2019). In fact, secondary bile acids such as LCA has been observed to protect mice against colitis by acting as an anti-inflammatory agent (Ward J. B. J. et al., 2017).

### Example 29

### (Effect of CLX 115 on Ex-Vivo Human Fecal Microbiota)

After observing a strong beneficial effect of CLX115 on fecal fermentations using a pool of feces, we decided to conduct fermentations with 15 samples from individual donors, to confirm the effect of CLX115 and explore responses at a community level. This experiment confirmed that CLX115 induced a robust production of SCFAs, especially butyrate, in several distinct complex microbial communities. Preparation of fecal slurry, fecal fermentation assay, DNA extractions and sequencing, as well as SCFA analysis were run as described in Example 24.

### Microbiota Analysis

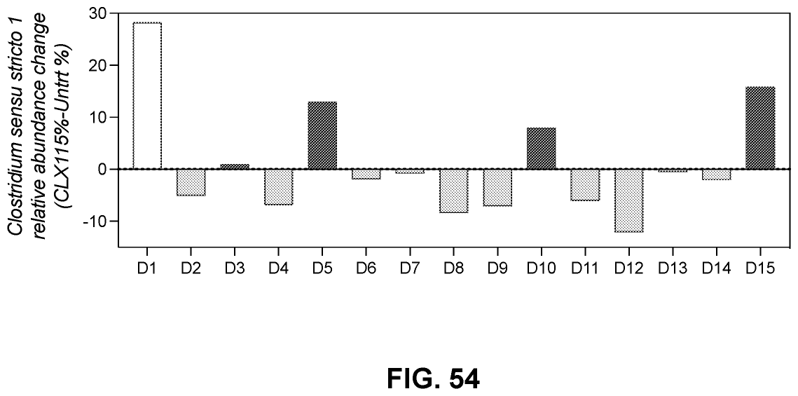

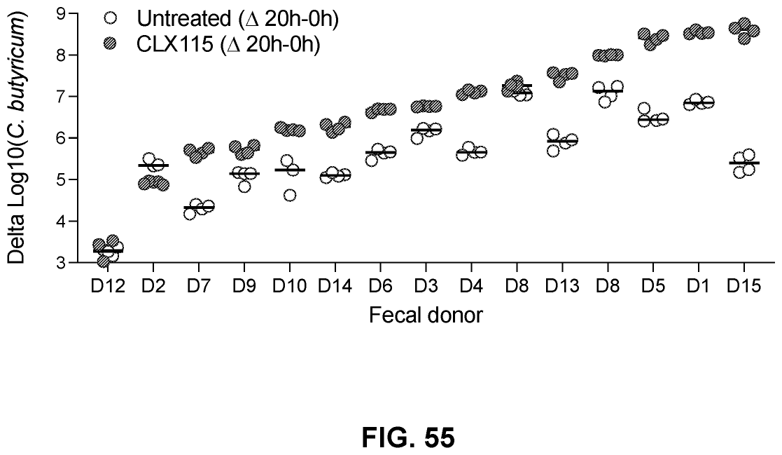

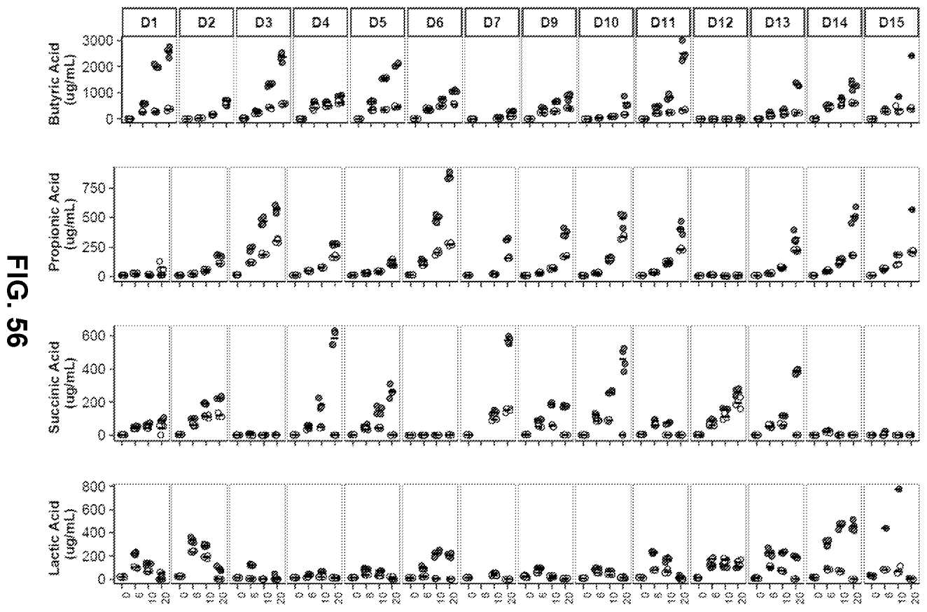

A generalized increase in the relative abundance of Firmicutes was observed in 13 of the 15 fecal samples. CLX115 supplementation resulted in an increase of one or multiple taxonomic groups previously recognized as butyrate producers. As a result of interindividual differences in the starting fecal microbial communities, different genera were enriched during fermentations in the presence of CLX115 oligosaccharides. The largest increase in relative abundance after 20 hour of fermentation was observed in the Clostridium sensu stricto 1 genus, in 31% (5 samples) of the tested population. This genus made up to 30% of the final population by the end of the incubation period (Table 50). Enrichment of this group was mainly due to the proliferation of Cl. butyricum which is consider a next generation probiotic and a well-recognized butyrate producer. Other genera that showed a significant increase include Blautia in 37.5% (6 samples), Erysipelatoclostridium in 37.5% (6 samples), Roseburia in 19% (3 samples), and Collinsella in 12.5% (2 samples) of the tested population (Table 50, FIG. 54). This attests of the functional redundancy of the gut microbial communities. Given the health and commercial relevance of Clostridium butyricum we decided to evaluate the response of this taxa as changes in absolute concentrations to overcome limitations of compositional data provided by 16S sequencing. Remarkably, absolute quantification by qPCR revealed that there is in fact an 80% responder rate for Cl. butyricum when CLX115 is provided as a carbon source (FIG. 55).

TABLE 50
Changes in relative abundance of microbes in oligo-treated samples versus
untreated control, in 15 fecal samples from individual donors and 2 pools.

| Taxonomic group | D1 | D2 | D3 | D4 | D5 | D6 | D7 | D8 | D9 | D10 | D11 | D12 | D13 | D14 | D15 |
|---|---|---|---|---|---|---|---|---|---|---|---|---|---|---|---|
| Firmicutes | + | + | + | + | + | + | − | + | + | + | + | − | + | + | + |
| Clostridiaceae | + | − | + | − | + | − | − | − | − | + | − | − | − | − | − |
| Clostridium sensu | + | − | + | − | + | − | − | − | − | + | − | − | − | − | − |
| stricto 1 | | | | | | | | | | | | | | | |
| Clostridium | + | + | + | + | + | + | + | + | − | + | − | + | − | − | − |
| butyricum | | | | | | | | | | | | | | | |
| Blautia | + | + | + | + | + | + | + | + | + | − | − | − | + | + | + |
| Roseburia | − | + | − | + | + | − | − | + | + | − | + | + | − | + | + |
| Erysipelatoclostridium | + | + | + | + | + | + | + | + | + | + | + | − | + | + | − |
| Bifidobacterium | + | − | − | − | − | − | + | − | − | − | − | + | − | − | − |
| Collinsella | + | − | − | − | + | + | − | − | + | − | + | + | + | − | + |
| Eggerthella | − | − | − | − | − | + | − | − | − | + | − | + | + | − | − |
| Bacteroides | − | − | − | − | − | + | − | − | + | − | − | + | − | − | − |
| Prevotella | − | − | − | − | + | + | − | + | + | − | − | + | + | − | + |
| Parabacteroides | − | − | − | − | + | + | + | − | + | − | − | + | − | − | + |
| Lactobacillus | + | − | + | − | − | + | − | − | + | + | − | − | − | + | + |
| Agathobacter | + | + | + | + | + | + | − | − | + | + | − | − | + | + | + |
| Proteobacteria | − | − | − | − | − | − | + | − | − | − | − | + | + | − | − |
| Akkermansia | − | − | − | − | − | − | − | − | − | + | − | − | + | − | − |

Key symbols:
+ = significant increase in relative abundance vs untreated control; − significant decrease in relative abundance vs untreated control.

### Short Chain Fatty Acid Analysis

The short chain fatty acid profiles of the single donor samples were analyzed to assess the bioconversion of the CLX115 composition into short chain fatty acid intermediate and end products. At least one of total short chain fatty acids, including butyrate, propionate, succinate and lactate, were higher in all but two samples supplemented with CLX115 when compared to the untreated samples (FIG. 56). The fermentations that did not result in higher SCFA production seem to have been a result of fecal microbial inactivity. This based on pH and GD measurements that revealed virtually no biomass production or metabolic activity throughout the fermentation in both treated and untreated samples.

When compared to the untreated control 100% of the active fecal samples (14 subjects) produced butyric acid after 20 h of fermentation when supplemented with CLX115. Short chain fatty acid profiles differed, due to different initial microbial populations in each donor sample. For example, D1 SCFA production was almost entirely butyric acid. Other subjects like D3, D6, D11, D15, and D16, produced large amounts of both butyric and propionic acid. Production of both large amount of butyrate and moderate levels of succinic acid was observed in D5. While D14 produced relatively large levels of butyric, propionate and succinate. Finally, D2, D7, D9, and D10 produced a moderate equivalent amount of butyrate, propionate and succinic acid. Distinct SCFA ratios reflect unique interindividual microbial taxonomic composition and their ecological interactions. CLX115 drives an overall beneficial modulation of taxonomic groups, enriching butyrate producers and producing large levels SCFAs. CLX115 also has the ability to produce remarkably robust responses of Clostridium butyricum despite differences in initial fecal inoculums.

### Example 30

Improvement of Oligosaccharide Production from Curdlan: Curdlan (550 mg) was dissolved in 12 ml of 55 mM Sodium acetate buffer at pH5.5 in a capped reaction vessel and placed in a shaker-zincubator for 20 min at 55° C. and 85 RPM. Hydrogen peroxide (3.7 ml) and iron (II) sulfate (2.9 mg in 50 μL water) were added to the reaction mixture and mixed thoroughly. The reaction in the capped reaction vessel proceeded in the shaker-incubator at 55° C. and 65 RPM for two hours. The capped reaction cooled to 12° C. in a −20° C. freezer. Ammonium hydroxide (0.42 ml of 28% v/v to pH 10.2) was used to adjust pH and sample was reacted at 45° C. in a shaker-incubator for 2 hours at 20 RPM, the cap was left loose to allow oxygen, ammonia, and carbon dioxide gases to be released. The sample was then frozen and lyophilized, then stored at −80° C. The freeze-dried oligosaccharide mixture was rehydrated with the minimum amount of water required to allow for a free-flowing solution. This solution was then loaded onto a column containing 5.5 mL mixed bed ion exchange resin per gram (dry weight) of crude material, and the runoff was collected in a plastic freezer bag. Once the material was loaded onto the column, the column was then rinsed with 3 bed volumes of water. Finally, the runoff was sealed and frozen in the bag, then carefully shattered and subjected to lyophilization.

Through the manipulation of reaction parameters, the generation of useful oligosaccharides was improved. The yield reaction was increased to 17%. Additionally, all chromatograms showed increased signal to noise ratios and greater diversity in oligosaccharides produced. While monosaccharide analysis remained nearly unchanged, linkage analysis showed increased break down. Furthermore, both Oligosaccharide and NMR analysis demonstrated increased diversity and prevalence of oligosaccharides. The oligosaccharide analysis results are set forth in in Table B2.

TABLE 51.1
Monosaccharide composition of claimed composition of matter pools.
Units represent relative abundance by mass. The notation “—”
represents a monosaccharide that exists in an amount less than
2% (which can be 0%) of the total polysaccharide weight.

| ID | Glucose | Arabinose | GlcA | Galactose | Mannose | Other |
|---|---|---|---|---|---|---|
| CLX101C | 93.3 | 4.72 | — | — | − | 1.66 |

TABLE 51.2
Glycosidic linkage compositions of claimed composition
of matter pools. Units represent relative abundance by
peak area. The notation “—” represents a glycosidic
linkage that exists in an amount less than 2% (which
can be 0%) of the total polysaccharide weight.

| ID | 3-Glc | T-Glc | 2,x-Hexose | Other |
|---|---|---|---|---|
| CLX101C | 69.61 | 18.18 | 5.49 | 6.72 |

TABLE 51.3
1H-13C HSQC NMR correlations from oligosaccharides created from
the COG process. The listed pairs correspond to those major peaks
in the anomeric region.
CLX 101C

| ¹H δ ppm | ¹³C δ ppm |
|---|---|
| 5.08 | 99.89 |
| 4.99 | 95.49 |
| 4.97 | 91.54 |
| 4.95 | 101.63 |
| 4.94 | 91.32 |
| 4.90 | 91.97 |
| 4.90 | 101.66 |
| 4.85 | 92.50 |
| 4.55 | 94.04 |
| 4.51 | 101.05 |
| 4.51 | 102.83 |
| 4.44 | 94.87 |
| 4.42 | 103.20 |
| 4.43 | 94.87 |
| 4.36 | 103.63 |
| 4.36 | 96.11 |
| 4.28 | 103.85 |
| 4.26 | 96.64 |
| 4.20 | 97.13 |
| 4.20 | 102.84 |
| 3.84 | 91.71 |
| 3.81 | 91.93 |

### Example 31

Improvement of Oligosaccharide Production from Yeast mannan extract: Yeast mannan extract (550 mg) was dissolved in 9.4 mL of 55 mM Sodium acetate buffer at pH5.5 in a capped reaction vessel and placed in a shaker-incubator for 20 min at 55° C. and 85 RPM. Hydrogen peroxide (2.85 ml) and iron (II) sulfate (3.90 mg in 50 μL water) were added to the reaction mixture and mixed thoroughly. The reaction in the capped reaction vessel proceeded in the shaker-incubator at 55° C. and 65 RPM for two hours. The capped reaction cooled to 12° C. in a −20° C. freezer. Ammonium hydroxide (0.331 ml of 28% v/v to pH 10.2) was used to adjust pH and sample was reacted at 45° C. in a shaker-incubator for 2 hours at 20 RPM, the cap was left loose to allow oxygen, ammonia, and carbon dioxide gases to be released. The sample was then frozen and lyophilized, then stored at −80° C. The freeze-dried oligosaccharide mixture was rehydrated with the minimum amount of water required to allow for a free-flowing solution. This solution was then loaded onto a column containing 6 mL mixed bed ion exchange resin per gram (dry weight) of crude material, and the runoff was collected in a plastic freezer bag. Once the material was loaded onto the column, the column was then rinsed with 3 bed volumes of water. Finally, the runoff was sealed and frozen in the bag, then carefully shattered and subjected to lyophilization.

Through the manipulation of reaction parameter, the generation of useful oligosaccharides was improved. The yield of the reaction increased to 35%. Additionally, all chromatograms generated showed increased signal to noise ratios and greater diversity in oligosaccharides produced. Monosaccharide analysis showed increase glucose while mannose decreased. Whereas linkage analysis showed increased complexity in oligosaccharides generated with a greater diversity in linkages seen. Furthermore, both Oligosaccharide and NMR analysis demonstrated increased prevalence of oligosaccharides. The oligosaccharide analysis results are set forth in in Table P2.

TABLE 52.1
Monosaccharide composition of claimed composition of matter pools.
Units represent relative abundance by mass. The notation “—”
represents a monosaccharide that exists in an amount less than
2% (which can be 0%) of the total polysaccharide weight.

| ID | Glucose | Mannose | Galactose | Arabinose | GlcA | Ribose | Other |
|---|---|---|---|---|---|---|---|
| CLX115AL | 42.4 | 52.33 | — | 1.19 | — | 2.63 | 1.21 |

TABLE 52.2
Glycosidic linkage compositions of claimed composition of matter pools. Units represent
relative abundance by peak area. The notation “—” represents a glycosidic linkage
that exists in an amount less than 2% (which can be 0%) of the total polysaccharide weight.

| ID | 3-Glc | 4-Glc | 6-Glc | T-Glc | 2-Man | 3-Man | 4-Man | T-Man | 5-Arab | T-Ribose | Other |
|---|---|---|---|---|---|---|---|---|---|---|---|
| CLX115AL | 3.95 | 25.15 | 9.09 | 7.84 | 24.97 | 2.21 | 2.22 | 6.74 | 2.59 | 3.98 | 11.27 |

TABLE 52.3
1H-13C HSQC NMR correlations from oligosaccharides created from
the COG process. The listed pairs correspond to those major peaks
in the anomeric region.
CLX 115AL

| ¹H δ ppm | ¹³C δ ppm |
|---|---|
| 4.99 | 92.50 |
| 4.97 | 100.63 |
| 4.93 | 91.73 |
| 4.86 | 101.75 |
| 4.86 | 92.17 |
| 4.83 | 93.88 |
| 4.81 | 102.04 |
| 4.68 | 99.12 |
| 4.50 | 94.16 |
| 4.47 | 103.06 |
| 4.39 | 103.40 |
| 4.33 | 96.32 |
| 4.32 | 103.82 |
| 4.25 | 103.93 |
| 4.22 | 96.86 |

### Example 32

Improvement of Oligosaccharide Production from Beta Glucan from oat: Beta Glucan (550 mg) was dissolved in 9.4 mL of 55 mM Sodium acetate buffer at pH5.5 in a capped reaction vessel and placed in a shaker-incubator for 20 min at 55° C. and 85 RPM. Hydrogen peroxide (3.06 ml), iron (III) sulfate (2.75 mg in 50 μL water) and Copper (II) sulfate (1.90 mg in 50 μL water) were added to the reaction mixture and mixed thoroughly. The reaction in the capped reaction vessel proceeded in the shaker-incubator at 55° C. and 65 RPM for three hours. The capped reaction cooled to 12° C. in a −20° C. freezer. Ammonium hydroxide (0.413 ml of 28% v/v to pH 10.2) was used to adjust pH and sample was reacted at 45° C. in a shaker-incubator for 1.5 hours at 20 RPM, the cap was left loose to allow oxygen, ammonia, and carbon dioxide gases to be released. The sample was then frozen and lyophilized, then stored at −80° C. The freeze-dried oligosaccharide mixture was rehydrated with the minimum amount of water required to allow for a free-flowing solution. This solution was then loaded onto a column containing 5.5 mL mixed bed ion exchange resin per gram (dry weight) of crude material, and the runoff was collected in a plastic freezer bag. Once the material was loaded onto the column, the column was then rinsed with 3 bed volumes of water. Finally, the runoff was sealed and frozen in the bag, then carefully shattered and subjected to lyophilization.

Through the manipulation of reaction parameter, the generation of useful oligosaccharides was improved. Yield increased to 43%. Additionally all chromatograms generated showed increased signal to noise ratios and greater diversity in oligosaccharides produced. While monosaccharide analysis remained nearly unchanged, linkage analysis showed increased break down. Furthermore, both Oligosaccharide and NMR analysis demonstrated increased diversity and prevalence of oligosaccharides. The oligosaccharide analysis results are set forth in in Table M2.

TABLE 53.1
Monosaccharide composition of claimed composition of matter pools.
Units represent relative abundance by mass. The notation “—”
represents a monosaccharide that exists in an amount less than
2% (which can be 0%) of the total polysaccharide weight.

| ID | Glucose | Arabinose | GlcA | Galactose | Mannose | Other |
|---|---|---|---|---|---|---|
| CLX115FC | 94.38 | 2.79 | — | — | — | 2.36 |

TABLE 53.2
Glycosidic linkage compositions of claimed composition
of matter pools. Units represent relative abundance by
peak area. The notation “—” represents a glycosidic
linkage that exists in an amount less than 2% (which
can be 0%) of the total polysaccharide weight.

| ID | 2-Glc | 3-Glc | 4-Glc | 6-Glc | T-Glc | T-Arab-f | Other |
|---|---|---|---|---|---|---|---|
| CLX115FC | — | 16.95 | 56.97 | — | 10.68 | 5.79 | 8.01 |

TABLE 53.3
1H-13C HSQC NMR correlations from oligosaccharides created from
the COG process. The listed pairs correspond to those major peaks
in the anomeric region.
CLX 115FC

| ¹H δ ppm | ¹³C δ ppm |
|---|---|
| 5.14 | 99.92 |
| 4.99 | 100.53 |
| 4.96 | 91.54 |
| 4.94 | 101.56 |
| 4.90 | 101.65 |
| 4.89 | 91.89 |
| 4.85 | 92.37 |
| 4.41 | 103.43 |
| 4.36 | 102.43 |
| 4.35 | 96.14 |
| 4.32 | 96.43 |
| 4.31 | 102.59 |
| 4.26 | 96.68 |
| 4.23 | 103.00 |
| 4.20 | 97.14 |
| 4.16 | 102.93 |

### Example 33

Improvement of Oligosaccharide Production from Pea Fiber: Destarched Pea Fiber (550 mg) was dissolved in 8.4 mL of 55 mM Sodium acetate buffer at pH5.5 in a capped reaction vessel and placed in a shaker-incubator for 20 min at 55° C. and 85 RPM. Hydrogen peroxide (2.57 ml) and iron (III) sulfate (4.60 mg in 50 μL water) were added to the reaction mixture and mixed thoroughly. The reaction in the capped reaction vessel proceeded in the shaker-incubator at 55° C. and 65 RPM for three hours. The capped reaction cooled to 12° C. in a −20° C. freezer. Ammonium hydroxide (0.27 ml of 28% v/v to pH 10.2) was used to adjust pH and sample was reacted at 45° C. in a shaker-incubator for 2 hours at 20 RPM, the cap was left loose to allow oxygen, ammonia, and carbon dioxide gases to be released. The sample was then frozen and lyophilized, then stored at −80° C. The freeze-dried oligosaccharide mixture was rehydrated with the minimum amount of water required to allow for a free-flowing solution. This solution was then loaded onto a column containing 5.5 mL mixed bed ion exchange resin per gram (dry weight) of crude material, and the runoff was collected in a plastic freezer bag. Once the material was loaded onto the column, the column was then rinsed with 3 bed volumes of water. Finally, the runoff was sealed and frozen in the bag, then carefully shattered and subjected to lyophilization.

Through the manipulation of reaction parameter, the generation of useful oligosaccharides was improved. The yield increased to 33%. Additionally all chromatograms generated showed increased signal to noise ratios and greater diversity in oligosaccharides produced. While monosaccharide analysis remained nearly unchanged with just an increase in arabinose prevalence, linkage analysis showed increased break down. Furthermore, both Oligosaccharide and NMR analysis demonstrated increased diversity and prevalence of oligosaccharides. The oligosaccharide analysis results are set forth in in Table U3.

TABLE 54.1
Monosaccharide composition of claimed composition of matter pools. Units represent
relative abundance by mass. The notation “—” represents a monosaccharide
that exists in an amount less than 2% (which can be 0%) of the total polysaccharide weight.

| ID | Glucose | Galactose | Mannose | Xylose | Arabinose | Rhamnose | GlcA | GalA | Other |
|---|---|---|---|---|---|---|---|---|---|
| CLX122DSF | 8.75 | 6.85 | — | 6.66 | 64.1 | 3.12 | — | 8.37 | 1.7 |

TABLE 54.2
Glycosidic linkage compositions of claimed composition of matter pools. Units represent
relative abundance by peak area. The notation “—” represents a glycosidic linkage
that exists in an amount less than 2% (which can be 0%) of the total polysaccharide weight.

| | | | | 3,5- | 2,5- | 2,3,5- | T- | | 2,3,4- | | |
|---|---|---|---|---|---|---|---|---|---|---|---|
| ID | 4-Glc | 4-Gal | 5-Arab | Arab | Arab | Arab | Arab-f | 2,4-Xyl | Xyl | T-Xyl | Other |
| CLX122DSF | — | 2.1 | 17 | 3.84 | 1.75 | — | 50.18 | 4.08 | 10.04 | 2 | 9.01 |

TABLE 54.3
1H-13C HSQC NMR correlations from oligosaccharides created from
the COG process. The listed pairs correspond to those major peaks
in the anomeric region.
CLX122DSF

| ¹H δ ppm | ¹³C δ ppm |
|---|---|
| 5.17 | 100.60 |
| 5.08 | 95.20 |
| 5.07 | 100.12 |
| 5.02 | 108.48 |
| 5.02 | 100.21 |
| 5.01 | 92.27 |
| 4.99 | 100.39 |
| 4.99 | 95.51 |
| 4.99 | 92.13 |
| 4.97 | 106.14 |
| 4.96 | 101.79 |
| 4.92 | 107.05 |
| 4.90 | 101.62 |
| 4.90 | 98.46 |
| 4.90 | 92.05 |
| 4.89 | 106.52 |
| 4.87 | 101.19 |
| 4.85 | 107.00 |
| 4.85 | 92.48 |
| 4.82 | 106.57 |
| 4.80 | 93.82 |
| 4.76 | 107.75 |
| 4.72 | 94.18 |
| 4.63 | 98.84 |
| 4.32 | 102.54 |
| 4.31 | 96.51 |
| 4.26 | 102.05 |
| 4.26 | 96.68 |
| 4.24 | 105.55 |
| 4.23 | 102.75 |
| 4.21 | 97.16 |

### Example 34

### (IBS Trial)

This example demonstrates how an IBS trial would be conducted to assess the effects of the oligosaccharide compositions and formulations thereof on subjects in a clinical setting.

A total of 60 male and female Irritable Bowel Syndrome (IBS) patients are recruited to participate in the study. After a screening visit and run-in period of 2 weeks, eligible patients are selected to participate in the study. The patients are randomized into two study groups, each of 30 patients, with one study group consuming the treatment product and one study group the placebo product. The study runs for a period of 12 weeks. The treatment product contains 5 grams of CLX 122DSF (or any other CLX composition disclosed herein or any combination thereof, or structurally similar variants thereof or any combination thereof), while the placebo product contains 5 grams glucose. Both products are in powder form in a unit dosage container.

The patients are eligible to participate in the study if they are at an age between 18-60 years, fulfil the definition of diarrhea predominant IBS (IBS-D), constipation predominant IBS (IBS-C) or alternating IBS (IBS-A), according to the Rome IV criteria for IBS and have a global IBS-SSS score of >174 during the 2 weeks run-in period. All recruited patients are able and willing to understand and comply with the study procedures. Patients are excluded if they: have any known gastrointestinal disease(s) that may cause symptoms or interfere with the study outcome, in particular lactose intolerance and coeliac disease; have participated in a clinical study one month prior to the screening visit; have abnormal results in the screening tests which are clinically relevant for study participation; are suffering from a severe disease such as malignancy, diabetes, severe coronary disease, kidney disease, neurological disease, or severe psychiatric disease or any condition which can confound the results of the study; have used highly dosed probiotic supplements (yogurt allowed) for 1 months prior to the study; have consumed antibiotic drugs 1 months prior to the study; have consumed on a regular basis any medication that might interfere with symptom evaluation 2 weeks prior to the study; have diagnosed with and treated for IBS for more than 10 years; and are pregnant or lactating.

At the screening visit, the patients are given both oral and written information about the study, are asked for informed assent and are asked to sign an informed consent form. Also, clinical and medical history and concomitant medication is registered. IBS diagnostic criteria are assessed and part 2 of the IBS-SSS questionnaire is completed. A fecal sample kit is distributed together with the Bristol Stool Form Scale (BSFS) and Bowel Movement Diary (BMD). The BSFS and BMD are to be filled in during the 7 days just prior to the second visit. Patients are asked to register their diet the 3 days just prior to the second visit and are reminded not to change their usual diet during the study.

At the second visit which is the start of the intervention, eligibility criteria are checked, and eligible subjects are randomized to the two arms in the trial. A physical examination is done, and the patients fill out the following questionnaires electronically: Gastrointestinal Symptom Rating Scale-IBS (GSRS-IBS), Irritable Bowel Syndrome Severity Scoring System (IBS-SSS), Hospital Anxiety and Depression Scale (HADS), Numeric Rating Scale-11 (NRS-11), Visceral Sensitivity Index (VSI), Irritable Bowel Syndrome Quality of Life (IBS-QOL) and Patient Health Questionnaire 15 (PHQ-15 scales) are filled in electronically. Those who are unable or unwilling to use the electronic system, fill out the questionnaires on paper. Based on clinical symptoms and data from questionnaires, patients are characterized into one of the three following IBS subgroups: diarrhea predominant (IBS-D), constipation predominant (IBS-C) or alternating (IBS-A). This enables even allocation of patients from each IBS subgroup into study groups. When allocated to the study groups, patients are provided with either treatment or placebo products. Patients are instructed to consume the products in the morning with breakfast. Compliance is monitored through the interactive internet enabled system. Patients are asked about any adverse events in the run-in period and any changes in their usual medication. The BSFS and BMD are collected and new forms, to be filled in daily during the intervention period, are distributed. Fecal samples are collected and equipment for new samples are distributed. Blood samples are collected for routine clinical chemistry and hematology and biomarker analysis. Diet records are collected, and patients are asked to register their diet for the 3 days just prior to the third visit. Patients are reminded not to change their usual diet during the study.

At the third visit which is 6 weeks after the second visit, a physical examination is performed and the same questionnaires (GSRS-IBS, IBS-SSS, HADS, NRS-11, VS I, IBS-QOL and PHQ-15 scales) are answered. The questionnaires are filled in electronically. Those who are unable or unwilling to use the electronic system fill out the questionnaires on paper. Blood samples are collected for routine clinical chemistry and hematology and biomarker analysis. Patients are asked about any adverse events and any changes in their usual medication. Fecal samples are collected and equipment for collecting new samples distributed. The BSFS and BMD are collected and new forms, to be filled in during the 7 days just prior to the fourth visit, are distributed. Diet records are collected, and patients are reminded not to change their usual diet during the study.

At the fourth visit which is 12 weeks after the second visit, a physical examination is performed and the same questionnaires (GSRS-IBS, IBS-SSS, HADS, NRS-11, VS I, IBS-QOL and PHQ-15 scales) are answered. The questionnaires are filled in electronically. Those who are unable or unwilling to use the electronic system fill out the questionnaires on paper. Blood samples are collected for routine clinical chemistry and hematology and biomarker analysis. Patients are asked about any adverse events and any changes in their usual medication. Fecal samples and BSFS and BMD are collected. Diet records are collected and each patient has an exit visit with the medical team. Remaining study products and compliance diaries are collected to check compliance.

To assess the intestinal microbiota profile, DNA is extracted from fecal samples using a 96-well PowerSoil DNA Isolation Kit (MO-BIO). A minimum of one sample-well per plate is kept empty to serve as a negative control during PCR. PCR is done with the forward primer S-D-Bact-0341-b-S-17 and reverse primer S-D-Bact-0785-a-A-21 using Illumina adapters (Klindworth et al. Nucleic Acids Res. 41, el (2013)). These are universal bacterial 16S rDNA primers, which target the V3-V4 region. The following PCR program is used: 98° C. for 30 sec, 25× (98° C. for 10 s, 55° C. for 20 s, 72° C. for 20 s), 72° C. for 5 min. Amplification is verified by running the products on a 1% agarose gel. Barcodes are added in a nested PCR using the Nextera Index Kit V2 (Illumina) with the following PCR program: 98° C. for 30 sec, 8× (98° C. for 10 s, 55° C. for 20 s, 72° C. for 20 s), 72° C. for 5 min. Attachment of primers is verified by running the products on a 1% agarose gel. Products from the nested PCR are normalized using the SequalPrep Normalization Plate Kit and pooled. Pooled libraries are concentrated by evaporation and the DNA concentration of pooled libraries is measured on a Qubit fluorometer using the Qubit High Sensitivity Assay Kit (Thermo Fisher Scientific). Sequencing is done on a MiSeq desktop sequencer using the MiSeq Reagent Kit V3 (Illumina) for 2×300 bp paired-end sequencing. The 64-bit version of USEARCH is used for bioinformatical analysis of the sequence data.

The results indicate that oral ingestion of 5 grams of CLX 122DSF (or any other CLX composition disclosed herein or any combination thereof, or structurally similar variants thereof or any combination thereof) modulates the intestinal microbiota, and specifically stimulate the abundance of bifidobacteria. The blood biomarker analysis indicates that the treatment patients have reduced levels of inflammatory markers. Reduction in the IBS-SSS score is reported by treatment group patients. The treatment patients indicate reduced pain severity and duration and an improvement in bowel movement as compared to the placebo group. The treatment group patients indicate an improvement in IBS-QOL and HADS scores as compared to the placebo group.

### Example 35

### (Diabetes Trial)

This example demonstrates how a diabetes trial would be conducted to assess the effects of the oligosaccharide compositions and formulations thereof on subjects in a clinical setting.

A total of 100 male and female diabetic patients with cardiovascular co-morbidities are recruited to participate in the study. Patients are randomized into two groups, each of 50 patients, with 1 group receiving a treatment product and one group receiving a placebo product. The treatment product contains 5 grams of CLX115FC (or any other CLX composition disclosed herein or any combination thereof, or structurally similar variants thereof or any combination thereof), while the placebo product contains 5 grams maltodextrin. All products are in powder form in a unit dosage container.

The patients are eligible to participate if: they are older than 45 years of age, have a BMI of 25 or above, have an A1C score of 6.5 or above or a fasting blood glucose level of 126 or above, have been diagnosed as a type II diabetic within the last 1 year, and have been diagnosed with a cardiovascular co-morbidity such as ischemic heart disease, coronary heart disease, atherosclerotic heart disease, atherosclerotic cardiovascular disease, stroke and transient ischemic attacks. All recruited patients are able and willing to understand and comply with the study procedures. Patients are excluded if: they have participated in a clinical study one month prior to the screening visit and throughout the study; have any disease(s) that may cause symptoms or may interfere with the trial outcome; have other severe disease(s) such as malignancy, kidney disease or neurological disease; have psychiatric disease; have used highly dosed probiotic supplements (yogurt allowed) 1 month prior to screening and throughout the study; have consumed antibiotic drugs 1 month prior to screening and throughout the study; and consume on a regular basis medication that might interfere with symptom evaluation 2 weeks prior to screening and throughout the study.

At an initial visit (screening), the patients are given both oral and written information about the study; the patients are asked for informed assent and the patients are asked to sign an informed consent form. Eligibility criteria are checked and for patients who are enrolled to the study, medical history and concomitant medication are registered. A physical examination is done. Blood pressure, pulse rate, height and bodyweight are measured, and body composition is determined by a DXA (dual energy x-ray absorptiometry)-scan and bioimpedance. BMI is calculated, waist and hip circumferences is measured, and food intake is registered. Medical history is checked for diabetes diagnosis and cardiovascular co-morbidity diagnosis. Fasting blood samples are collected and assessed for a fasting blood glucose level, safety and biomarker studies and for biobanking. The serum from the blood samples is transferred to cryotubes and stored at −80° C. The following biomarkers are measured; Lipopolysaccharides (LPS), hsC P, free fatty acids, total cholesterol, HDL, LDL, HbA1c, glucose, insulin, triglycerides, TNF-α, IL-Iβ, IL-6, IL-8, IL-10, GLP-1, GLP-2, Adiponectin, and Zonulin. Equipment for collecting fecal samples is distributed. The fecal samples are stored at −80° C. until analysis. Microbiological analysis is performed on the fecal samples using 16S rRNA gene sequencing. The Gastrointestinal Symptom Rating Scale (GSRS) is completed, and the Bristol Stool Form Scales (BSFS) is distributed to the patients with instructions to assess the stool consistency at each fecal sampling point using the BSFS.

At the second visit (randomization), the patients are asked about adverse events, fecal samples are collected and equipment for collection of new samples is distributed. BSFS is collected and a new BSFS is distributed. Study products are distributed together with a compliance form (diary). Patients reminded to follow their normal dietary habits.

The study runs for 16 weeks with the patients consuming either a placebo or the treatment product daily. Patients are instructed to consume the products in the morning with breakfast. Compliance is monitored via a compliance form (diary) to be filled in daily.

Eight weeks after commencement of the study, patient come in for a third visit. The patients are asked about adverse events and any changes in the patient's usual medication. Fecal samples are collected and equipment for collection of new samples is distributed. Blood pressure, pulse rate, waist and hip circumference, height and bodyweight are measured, and BMI calculated. The GSRS questionnaire is completed on site. The BSFS is collected and a new BSFS is distributed stool consistency at each fecal sampling point using the BSFS. Fasting blood samples are collected and assessed for a fasting blood glucose level, safety and biomarker studies and for biobanking. The serum from the blood samples is transferred to cryotubes and stored at −80° C. The following biomarkers are measured; Lipopolysaccharides (LPS), hsC P, free fatty acids, total cholesterol, HDL, LDL, HbA1c, glucose, insulin, triglycerides, TNF-α, IL-1β, IL-6, IL-8, IL-10, GLP-1, GLP-2, Adiponectin, and Zonulin. Patients are reminded to follow the healthy dietary habits.

At the end of intervention (16 weeks), each patient has a visit with the medical team. Patients and their representatives are asked about adverse events and any changes in the patient's usual medication. Study products and compliance forms are collected to check compliance. BSFS and fecal samples are collected. A physical examination is done. Blood pressure, pulse rate, height and bodyweight are measured, and body composition is determined by a DXA (dual energy x-ray absorptiometry)-scan and bioimpedance. BMI SDS is calculated, waist and hip circumferences measured, and food intake registered. Fasting blood samples are collected for safety and biomarker studies and for biobanking. Fasting blood samples are collected and assessed for a fasting blood glucose level, safety and biomarker studies and for biobanking. The serum from the blood samples is transferred to cryotubes and stored at −80° C. The following biomarkers are measured; Lipopolysaccharides (LPS), hsC P, free fatty acids, total cholesterol, HDL, LDL, HbA1c, glucose, insulin, triglycerides, TNF-α, IL-1β, IL-6, IL-8, IL-10, GLP-1, GLP-2, Adiponectin, and Zonulin. The GSRS questionnaire is completed on site by the patients.

To assess the intestinal microbiota profile, DNA is extracted from fecal samples using a 96-well PowerSoil DNA Isolation Kit (MO-BIG). A minimum of one sample-well per plate is kept empty to serve as a negative control during PCR. PCR is done with the forward primer S-D-Bact-0341-b-S-17 and reverse primer S-D-Bact-0785-a-A-21 using Illumina adapters (Klindworth et al. Nucleic Acids Res. 41, el (2013)). These are universal bacterial 16S rDNA primers, which target the V3-V4 region. The following PCR program is used: 98° C. for 30 sec, 25× (98° C. for 10 s, 55° C. for 20 s, 72° C. for 20 s), 72° C. for 5 min. Amplification is verified by running the products on a 1% agarose gel. Barcodes are added in a nested PCR using the Nextera Index Kit V2 (Illumina) with the following PCR program: 98° C. for 30 sec, 8× (98° C. for 10 s, 55° C. for 20 s, 72° C. for 20 s), 72° C. for 5 min. Attachment of primers is verified by running the products on a 1% agarose gel. Products from the nested PCR are normalized using the SequalPrep Normalization Plate Kit and pooled. Pooled libraries are concentrated by evaporation and the DNA concentration of pooled libraries is measured on a Qubit fluorometer using the Qubit High Sensitivity Assay Kit (Thermo Fisher Scientific). Sequencing is done on a MiSeq desktop sequencer using the MiSeq Reagent Kit V3 (Illumina) for 2×300 bp paired-end sequencing. The 64-bit version of USEARCH is used for bioinformatical analysis of the sequence data.

The results indicate that the treatment product contributes to reduced A1C, fasting blood glucose levels, and dyslipidemia. Further, the patients given the treatment product show a reduction of body fat, body weight and BMI as compared to the placebo group. The blood biomarker analysis indicates that the patients given the treatment product have increased levels of GLP-1 and GLP-2, reduced levels of metabolic endotoxemia and inflammatory markers and reduced gut permeability indicating an improved mucosal barrier compared to the placebo group. The fecal analysis indicates that the patients given the treatment product have increased abundance of butyrate producers such as Clostridium butyricum, and increased concentrations of butyrate.

All references listed below, or anywhere else throughout this description, are hereby incorporated by reference herein in their entireties for all purposes:
Amicucci, M. J., Galermo, A. G., et al. (2019). “A rapid-throughput adaptable method for determining the monosaccharide composition of polysaccharides.” International Journal of Mass Spectrometry 438: 22-28.
Amicucci, M. J., et al. (2019). “Function without Structures: The Need for In-Depth Analysis of Dietary Carbohydrates.” Journal of Agricultural and Food Chemistry 67(16): 4418-4424.
Amicucci, M. J., Park, D., et al. (2018). Production of bioactive oligosaccharides, WO 2018/236917.
Amicucci, M. J., Nandita, E., et al. (2020). “A nonenzymatic method for cleaving polysaccharides to yield oligosaccharides for structural analysis.” Nature Communications 11(1): 1-12.
Bar-On, Y. M., et al. (2018). “The biomass distribution on Earth.” Proceedings of the National Academy of Sciences 115(25): 6506-6511.
Bauer, S., et al. (2006). “Development and application of a suite of polysaccharide-degrading enzymes for analyzing plant cell walls.” Proceedings of the National Academy of Sciences 103(30): 11417-11422.
Barrett E., Ross R. P., O'Toole P. W., Fitzgerald G. F., Stanton C. Gamma-aminobutyric acid production by culturable bacteria from the human intestine. J. Appl. Microbio. 2012; 113:411-417.
Beckmann L., et al. (2006). “Isolation and identification of mixed linked β-glucan degrading bacteria in the intestine of broiler chickens and partial characterization of respective 1, 3-1, 4-β-glucanase activities.” Journal of Basic Microbiology 46(3): 175-185.
Byun, R., et al. (2004). “Quantitative Analysis of Diverse Latobacillus Species Present in Advanced Dental Caries.” Journal of Clinical Microbiology: 3128-3136.
Cancilla, M. T., et al. (1998). “Alkaline degradation of oligosaccharides coupled with matrix-assisted laser desorption/ionization Fourier transform mass spectrometry: a method for sequencing oligosaccharides.” Analytical Chemistry 70(4): 663-672.
Cani P. D, Bibiloni R., Knauf C., Waget A., Neyrinck A. M, Delzenne N. M, Burcelin R (2008). “Changes in gut microbiota control metabolic endotoxemia-induced inflammation in high-fat diet-induced obesity and diabetes in mice.” Gut. 57:1470-81.
Cani P. D., Possemiers S I, Van de Wiele T., Guiot Y., Everard A., Rottier O., Geurts L., Naslain D., Neyrinck A., Lambert D. M., Muccioli G. G., Delzenne N. M (2009). “Changes in gut microbiota control inflammation in obese mice through a mechanism involving GLP-1-driven improvement of gut permeability.” Gut. 58:1091-103.
Cavaglieri C R, Nishiyama A, Fernandes L C, et al. Differential effects of short-chain fatty acids on proliferation and production of pro- and anti-inflammatory cytokines by cultured lymphocytes. Life Sci. 2003;73: 1683-90.
Chambers E. S., Preston T., Frost G., Morrison D. J. (2008). “Role of gut microbiota-generated short-chain fatty acids in metabolic and cardiovascular health.” Curr Nutr Rep. 7:198-206.
Collins et al. (2018). “Promising Prebiotic Candidate Established by Evaluation of Lactitol, Lactulose, Raffinose, and Oligofructose for Maintenance of a Lactobacillus-Dominated Vaginal Microbiota.” Appl. Environ. Microbiol. doi: 10.1128/AEM.02200-17.
Desai, M. S., et al. (2016). “A dietary fiber-deprived gut microbiota degrades the colonic mucus barrier and enhances pathogen susceptibility.” Cell 167(5): 1339-1353.
Dominguez, A. L., et al. (2014). “An overview of the recent developments on fructooligosaccharide production and applications.” Food and Bioprocess Technology 7(2): 324-337.
Frei R., Lauener R. P., Crameri R., O'Mahony L (2012). “Microbiota and dietary interactions: an update to the hygiene hypothesis?” Allergy. 67:451-61.
Froslie K F, Jahnsen J, Moum B A, Vatn M H, IBSEN Group. Mucosal healing in inflammatory bowel disease: results from a Norwegian population-based cohort. Gastroenterology 2007; 133:412-22.
Galenro, A. G., Nandita. E., et al. (2018). “Liquid chromatography—tandem mass spectrometry approach for determining glycosidic linkages.” Analytical Chemistry 90(21): 13073-13080.
Galermo, A. G., Nandita, E., et al. (2019). “Development of an extensive linkage library for characterization of carbohydrates.” Analytical Chemistry 91(20): 13022-13031.
Gosling, A., Stevens, G. W., et al. (2010). “Recent advances refining galactooligosaccharide production from lactose.” Food Chemistry 121(2): 307-318.
Guarner, F. and Malagelada, J. R. (2003). “Gut flora in health and disease.” The Lancet 361(9356): 512-519.
Hamaker, B. R. and Tuncil, Y. E. (2014). “A perspective on the complexity of dietary fiber structures and their potential effect on the gut microbiota.” Journal of Molecular Biology 426(23): 3838-3850.
Hinz, S., et al. (2005). “Bifidobacterium longum endogalactanase liberate galactotriose from Type galactans.” Applied and Environmental Microbiology 71(9): 5501-5510.
Hobby G. P., Karaduta O., Dusio G. F., Singh M., Zybailov B. L., and J. M. Arthur. Chronic kidney disease and the gut microbiome. Am J. Physiol. Renal Physiol. 2019; 316: F1211-17.
Jausovec, D., et al. (2015). “Introduction of aldehyde vs. carboxylic groups to cellulose nanofibers using laccase/TEMPO mediated oxidation.” Carbohydrate Polymers 116: 74-85.
Kanbay M., Onal E., Afsar B., Dagel T., Yerlikaya A., Covic A., Vaziri N. D. The crosstalk of gut microbiota and chronic kidney disease: role of inflammation, proteinuria, hypertension, and diabetes mellitus. International Urology and Nephrology. 2018.
Kanoi T., Mikami Y., Hayashi A. A breakthrough in probiotics: Clostridium butyricum regulates gut homeostasis and anti-inflammatory response in inflammatory bowel disease. J Gastroenterol 2015; 50:928-39.
Kawaguchi, K., et al. (2014). “The mannobiose-forming exo-mannanase involved in a new mannan catabolic pathway in Bacteroides fragilis.” Archives of Microbiology 196(1): 17-23.
Kim Y., Shin C. The microbiota-gut-brain axis in neuropsychiatric disorders: Pathophysiological mechanisms and novel treatments. Curr Neuropharmacol. 2018; 16:559-573.
Kriaa A., Bourgin M., Potiron A., Mkaouar H., Jablaoui A., Gerard P., Maguin E., Rhimi M. (2019). “Microbial impact on cholesterol and bile acid metabolism: current status and future prospects.” J. Lipid Res. 60:323-332.
Kulcinskaja, E., et al. (2013). “Expression and characterization of a Bifidobacterium adolescentis beta-mannanase carrying mannan-binding and cell association motifs.” Applied and Environmental Microbiology 79(1): 133-140.
Lammerts van Bueren, A., et al. (2017). “Prebiotic galactoligosaccharides activate mucin and pectin galactan utilization pathways in the human gut symbiont Bacteroides thetaiotaomicron.” Scientific Reports 7(1): 1-13.
Larsbrink, J., et al. (2014). “A discrete genetic locus confers xyloglucan metabolism in select human gut Bacteroidetes.” Nature 506(7489): 498-502.
Leanti La Rosa, S., et al. (2019). “The human gut Firmicute Roseburia intestinalis is a primary degrader of dietary $-mannans.” Nature Communication 10(1): 1-14.
Li J., Kong D., Wang Q., Wu W., Tang Y., Bai T., Guo L., Wei L., Zhang Q., Yu Y., Qian Y., Zuo S., Liu Q., Wu S., Zang, Q Zhu, jia D., Want Y., Yao W., Ji Y., Yin, H., Nakamura M., Lazarus M., Breyer R. M., Want L., Yu Y. Niacin amerliorates ulcerative colitis via prostaglandin D-mediated D protanoid receptor 1 activation. EMBO Mol Med. 2017; 9:571-88.
LoCascio, R. G., Ninonuevo, M. R., et al. (2007). “Glycoprofiling of bifidobacterial consumption of human milk oligosaccharides demonstrates strain specific, preferential consumption of small chain glycans secreted in early human lactation.” Journal of Agricultural and Food Chemistry 55(22): 8914-8919.
Luis, A. S., et al. (2018). “Dietary pectin glycans are degraded by coordinated enzyme pathways in human colonic Bacteroides.” Nature Microbiology 3(2): 210-219.
Luu M., Monning H., Visekruna A (2020). “Exploring the molecular mechanisms underlying the protective effects of microbial SCFAs on intestinal tolerance and food allergy.” Front Immunol. 11:1225.
Ma X., Sun Q., Sun X., Chen D. Wei C., Yu X. C. Liu, Li Y. Li J., Activation of GABA receptors in colon epithelium exacerbates acute colitis. Front Immunol. 2018; 9:987.
Margolles, A. and De Los Reyes-Gavilin, C. G. (2003). “Purification and functional characterization of a novel alpha-L-arabinofuranosidase from Bifidobacterium longum B667.” Applied and Environmental Microbiology 69(9): 5096-5103.
Markus F Neurath, Simon P L Travis. Mucosal healing in inflammatory bowel diseases: a systematic review. Gut. 2019; 61: 1619-35.
Martens, E. C., et al. (2011). “Recognition and Degradation of Plant Cell Wall Polysaccharides by Two Human Gut Symbionts.” PLoS Biology 9(12), p.e1001221.
Merighi, M., McCoy J. M., et al. (2016). Biosynthesis of human milk oligosaccharides in engineered bacteria, Google Patents.
Mirjam A. C. Looijer-van Langen, Levinus A. Dieleman. Prebiotics in Chronic Intestinal Inflammation. Inflamm Bowel Dis. 2009; 15: 454-63.
Morrow, A. L., Ruiz-Palacios, G. M., et al. (2004). “Human milk oligosaccharides are associated with protection against diarrhea in breast-fed infants.” The Journal of Pediatrics 145(3): 297-303.
Mukhopadhya I, Hansen R. El-Omar E. M, Hold G. L. IBD-what role do Proteobacteria play? Nature Reviews, Gastroenterology & Hepatology. 2012 9: 219-230.
Mullish B H, McDonald J A K, Pechlivanis A, Allegretti J R, Kao D, Barker G F, et al. Microbial bile salt hydrolases mediate the efficacy of faecal microbiota transplant in the treatment of recurrent Clostridioides difficile infection. Gut. 2019; 68:1791-800. https://doi.org/10.1136/gutjnl-2018-317842.
Needham B. D., Kaddurah-Daouk R., Mazmanian S. K. Gut microbial molecules in behavioral and neurogenerative conditions. Nature Reviews Neuroscience. 2020; 21.
Neyrinck, A. M. et al (2012). “Wheat-derived arabinoxylan oligosacchiarides with prebiotic effect increase satietogenic gut peptides and reduce metabolic endotoxemia in diet-induced obese mice.” Nutrition & Diabetes 2(1): pp.e28-e28.
Pauly, M., et al. (1999). “A xyloglucan-specific endo-β-1, 4-glucanase from Aspergillus aculeatus: expression cloning in yeast, purification and characterization of the recombinant enzyme.” Glycobiology 9(1): 93-100.
Reddy, S. K., et al. (2016). “A β-mannan utilization locus in Bacteroides ovatus involves a GH36 α-galactosidase active on galactomannans.” FEBS Lett 590(14): 2106-2118.
Ren Z., Fan Y., Li An., Shen Q., Wu J., Ren L., Lu H., Ding S., Ren H., Liu C., Liu W., Gao D., Wu Z., Guo S., Wu G., Liu Z., Yu Z., Li L. Alterations of the human gut microbiome in Chronic Kidney Disease. Advanced Science. 2020, 7:2001936.
Rousseau et al. (2005). “Prebiotic effects of oligosaccharides on selected vaginal lactobacilli and pathogenic microorganisms.” Anaerobe, 11: 145-153.
Ruiz-Moyano, S., et al. (2013). “Variation in consumption of human milk oligosaccharides by infant gut-associated strains of Bifidobacterium breve.” Applied and Environmental Microbiology 79(19): 6040-6049.
S V Lynch (2016). “Gut microbiota and allergic disease.” New insight. Ann Am thorac Soc. 13:S51-S54.
Salyers, A. A., et al. (1977). “Fermentation of mucin and plant polysaccharides by strains of Bacteroides from the human colon.” Applied and Environmental Microbiology 33(2): 319-322.
Sartor R B. Therapeutic manipulation of the enteric microflora in inflammatory bowel diseases: antibiotics, probiotics, and prebiotics. Gastroenterology. 2004; 126:1 620-33.
Sheridan, P. O., et al. (2016). “Polysaccharide utilization loci and nutrition specialization in a dominant group of butyrate-producing human colonic Firmicutes.” Microbial Genomics 2(2).
Sikalidis A., Maykish A (2020). “The gut microbiome and type 2 diabetes mellitus: discussing a complex relationship.” Biomedicines. 8:8.
Smilowitz, J. T., Lebrilla, C. B., et al. (2014). “Breast milk oligosaccharides: structure-function relationships in the neonate.” Annual Review of Nutrition 34: 143-169.
Stadtman, E. and B. Berlett (1991). “Fenton chemistry. Amino acid oxidation.” Journal of Biological Chemistry 266(26): 17201-17211.
Strandwitz. Neurotransmitter modulation by the gut microbiota. Brain Res. 2018
Sun, B., et al. (2015). “Sodium periodate oxidation of cellulose nanocrystal and its application as a paper wet strength additive.” Cellulose 22(2): 1135-1146.
Tamura, K., et al. (2017). “Molecular Mechanism by which Prominent Human Gut Bacteroidetes Utilize Mixed-Linkage β-Glucans, Major Health-Promoting Cereal Polysaccharides.” Cell Reports 21(2): 417-430.
Turnbaugh P. J, Ley R. E, Mahowald M. A, Magrini V., Mardis E. R., Gordon J. I (2006.). “An obesity-associated gut microbiome with increased capacity for energy harvest.” Nature. 444:1027-31.
Van Laere, K. M., et al. (1999). “Transglycosidase activity of Bifidobacterium adolescents DSM 20083 alpha-galactosidase.” Applied Microbiology and Biotechnology 52(5): 681-688.
Wang, N., et al. (2016). “A review on Fenton-like processes for organic wastewater treatment.” Journal of Environmental Chemical Engineering 4(1): 762-787.
Ward J B J, Lajczak N K, Kelly O B, O'Dwyer A M, Giddam A K, Ni Gabhann J, et al. Ursodeoxycholic acid and lithocholic acid exert anti-inflammatory actions in the colon. Am J Physiol. 2017;312:G550-8 Available from: https://www.physiology.org/doi/10.1152/ajpgi.00256.2016. Accessed 23 Nov. 2020.
Wardman, P. and Candeias, L. P. (1996). “Fenton chemistry: an introduction.” Radiation Research 145(5): 523-531.
Wong J., Piceno Y. M., De Santis T. Z., Pahl M., Andersen G. L. Expansion of urease- and uricase-containing, indole- and p-cresol forming and contraction of short chain fatty acid producing intestinal microbiota in ESRD. Am J Nephrol 39: 230-237.
Yu, S., Liu, J.-J., et al. (2018). “Production of a human milk oligosaccharide 2′-fucosyllactose by metabolically engineered Saccharomyces cerevisiae.” Microbial Cell Factories 17(1): 101.

### STATEMENTS REGARDING INCORPORATION BY REFERENCE AND VARIATIONS

All references throughout this application, for example patent documents including issued or granted patents or equivalents; patent application publications; and non-patent literature documents or other source material; are hereby incorporated by reference herein in their entireties, as though individually incorporated by reference, to the extent each reference is at least partially not inconsistent with the disclosure in this application (for example, a reference that is partially inconsistent is incorporated by reference except for the partially inconsistent portion of the reference). All patents and publications mentioned in the specification are indicative of the levels of skill of those skilled in the art to which the invention pertains. References cited herein are incorporated by reference herein in their entirety to indicate the state of the art, in some cases as of their filing date, and it is intended that this information can be employed herein, if needed, to exclude (e.g., to disclaim) specific embodiments that are in the prior art. For example, when a compound is claimed, it should be understood that compounds known in the prior art, including certain compounds disclosed in the references disclosed herein (particularly in referenced patent documents), are not intended to be included in the claim.

The terms and expressions which have been employed herein are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments, exemplary embodiments and optional features, modification and variation of the concepts herein disclosed may be resorted to by those skilled in the art, and that such modifications and variations are considered to be within the scope of this invention as defined by the appended claims. The specific embodiments provided herein are examples of useful embodiments of the present invention and it will be apparent to one skilled in the art that the present invention may be carried out using a large number of variations of the devices, device components, methods steps set forth in the present description. As will be obvious to one of skill in the art, methods and devices useful for the present methods can include a large number of optional composition and processing elements and steps.

As used herein and in the appended claims, the singular forms “a”, “an”, and “the” include plural reference unless the context clearly dictates otherwise. Thus, for example, reference to “a cell” includes a plurality of such cells and equivalents thereof known to those skilled in the art. As well, the terms “a” (or “an”), “one or more” and “at least one” can be used interchangeably herein. It is also to be noted that the terms “comprising”, “including”, and “having” can be used interchangeably. The expression “of any of claims XX-YY” (wherein XX and YY refer to claim numbers) is intended to provide a multiple dependent claim in the alternative form, and in some embodiments is interchangeable with the expression “as in any one of claims XX-YY.”

When a group of substituents is disclosed herein, it is understood that all individual members of that group and all subgroups, including any isomers, enantiomers, and diastereomers of the group members, are disclosed separately. When a Markush group or other grouping is used herein, all individual members of the group and all combinations and subcombinations possible of the group are intended to be individually included in the disclosure. When a compound is described herein such that a particular isomer, enantiomer or diastereomer of the compound is not specified, for example, in a formula or in a chemical name, that description is intended to include each isomers and enantiomer of the compound described individual or in any combination. Additionally, unless otherwise specified, all isotopic variants of compounds disclosed herein are intended to be encompassed by the disclosure. For example, it will be understood that any one or more hydrogens in a molecule disclosed can be replaced with deuterium or tritium. Isotopic variants of a molecule are generally useful as standards in assays for the molecule and in chemical and biological research related to the molecule or its use. Methods for making such isotopic variants are known in the art. Specific names of compounds are intended to be exemplary, as it is known that one of ordinary skill in the art can name the same compounds differently.

Certain molecules disclosed herein may contain one or more ionizable groups [groups from which a proton can be removed (e.g., —COOH) or added (e.g., amines) or which can be quaternized (e.g., amines)]. All possible ionic forms of such molecules and salts thereof are intended to be included individually in the disclosure herein. With regard to salts of the compounds herein, one of ordinary skill in the art can select from among a wide variety of available counterions those that are appropriate for preparation of salts of this invention for a given application. In specific applications, the selection of a given anion or cation for preparation of a salt may result in increased or decreased solubility of that salt.

Every device, system, formulation, combination of components, or method described or exemplified herein can be used to practice the invention, unless otherwise stated.

Whenever a range is given in the specification, for example, a temperature range, a time range, or a composition or concentration range, all intermediate ranges and subranges, as well as all individual values included in the ranges given are intended to be included in the disclosure. It will be understood that any subranges or individual values in a range or subrange that are included in the description herein can be excluded from the claims herein.

All patents and publications mentioned in the specification are indicative of the levels of skill of those skilled in the art to which the invention pertains. References cited herein are incorporated by reference herein in their entirety to indicate the state of the art as of their publication or filing date and it is intended that this information can be employed herein, if needed, to exclude specific embodiments that are in the prior art. For example, when composition of matter are claimed, it should be understood that compounds known and available in the art prior to Applicant's invention, including compounds for which an enabling disclosure is provided in the references cited herein, are not intended to be included in the composition of matter claims herein.

As used herein, “comprising” is synonymous with “including,” “containing,” or “characterized by,” and is inclusive or open-ended and does not exclude additional, unrecited elements or method steps. As used herein, “consisting of” excludes any element, step, or ingredient not specified in the claim element. As used herein, “consisting essentially of” does not exclude materials or steps that do not materially affect the basic and novel characteristics of the claim. In each instance herein any of the terms “comprising”, “consisting essentially of” and “consisting of” may be replaced with either of the other two terms. The invention illustratively described herein suitably may be practiced in the absence of any element or elements, limitation or limitations which is not specifically disclosed herein.

One of ordinary skill in the art will appreciate that starting materials, biological materials, reagents, synthetic methods, purification methods, analytical methods, assay methods, and biological methods other than those specifically exemplified can be employed in the practice of the invention without resort to undue experimentation. All art-known functional equivalents, of any such materials and methods are intended to be included in this invention. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

1. A method for modulating microbiota to produce at least one short chain fatty acid and/or to increase an abundance of the microbiota, the method comprising:
contacting the microbiota with a formulation comprising an oligosaccharide composition;
wherein the method modulates the microbiota to produce the at least one short chain fatty acid and/or increase the abundance of the microbiota; and
wherein the oligosaccharide composition comprises:
(1) at least one first feature comprising:
a sum of glucose, galactose, and mannose subunits in an amount of at least 40 wt. %, based on total weight of saccharide subunits; or
(2) at least one second feature comprising:
a sum of rhamnose, galacturonic acid, arabinose, fucose, and mannose subunits in an amount of at least 3 wt. %, based on total weight of saccharide subunits;
a sum of xylose subunits in an amount of at least 33 wt. %, based on total weight of saccharide subunits;
a sum of mannose subunits in an amount of at least 50 wt. %, based on total weight of saccharide subunits; and a sum of galactose subunits in an amount of less than 20 wt. %, based on total weight of saccharide subunits;
a weight ratio of mannose subunits to glucose subunits between 2:1 and 4:1; and wherein at least 70 wt. % of the non-terminal mannose subunits have at least one 2-linkage, and wherein at least 70 wt. % of the non-terminal glucose subunits have at least one 3-linkage, at least one 4-linkage, and/or at least one 6-linkage; or
any combination thereof; or
(3) at least one third feature comprising:
a sum of glucose, galactose, mannose subunits in an amount of at least 50 wt. %, based on total weight of saccharide subunits;
a sum of glucose subunits in an amount of at least 20 wt. %, based on total weight of saccharide subunits; and a sum of xylose and arabinose subunits in an amount of at least 33 wt. %, based on total weight of saccharide subunits; or
any combination thereof.

2.-4. (canceled)

5. The method of claim 1, wherein the oligosaccharide composition comprises the at least one first feature.

6. The method of claim 5, wherein the oligosaccharide composition comprises:
a sum of arabinose subunits in an amount of less than 5 wt. %, based on total weight of saccharide subunits; and
less than 10 wt. % 2-linked mannose subunits, based on total weight of saccharide subunits.

7.-8. (canceled)

9. The method of claim 5, wherein the oligosaccharide composition comprises non-terminal glucose subunits, wherein (1) 20 wt. % to 100 wt. % of the non-terminal glucose subunits have at least one 3-linkage, based on total weight of non-terminal saccharide subunits; and/or (2) 33 wt. % to 80 wt. % of the non-terminal glucose subunits have at least one 4-linkage, based on total weight of non-terminal saccharide subunits.

10.-11. (canceled)

12. The method of claim 5, wherein the oligosaccharide composition comprises a weight ratio of glucose subunits having at least one 4-linkage to glucose subunits having at least one 3-linkage of between 2:1 to 4:1.

13.-14. (canceled)

15. The method of claim 5, wherein the oligosaccharide composition comprises at least one of: (a) at least two different isomers of 3Hex, (b) at least three different isomers of 3Hex, (c) at least four different isomers of 4Hex, (d) at least five different isomers of 5Hex, (e) at least three different isomers of 6Hex, or (f) any combination thereof.

16. (canceled)

17. The method of claim 5, wherein the oligosaccharide composition is derived from a material comprising:
Alcaligenes faecalis, konjac, locust bean, Icelandic moss, carob, barley, tamarind, oat, or any combination thereof; or
curdlan, glucomannan, galactomannan, lichenin, cereal beta glucan, xyloglucan, or any combination thereof.

18. (canceled)

19. The method of claim 5, wherein the oligosaccharide composition comprises:
CLX101, CLX102, CLX108, CLX110, CLX111, CLX112, CLX113, CLX115, CLX115-FC, CLX115-AL, or any combination thereof.

20. The method of claim 5, wherein the microbiota comprise:
Clostridium cluster I, optionally selected from Clostridium butyricum;
Clostridium cluster IV, optionally selected from Faecalibacterium prausnitzii;
Clostridium cluster XIVa, optionally selected from Roseburia spp, Eubacterium rectale, Eubacterium hallii, Blautia, Ruminococcus, Dorea, Coprococcus, or any combination thereof; or
any combination thereof.

21. The method of claim 5, wherein the method modulates the microbiota to produce the at least one short chain fatty acid, wherein the at least one short chain fatty acid comprises butyrate.

22. The method of claim 1, wherein the oligosaccharide composition comprises the at least one second feature.

23. The method of claim 22, wherein the oligosaccharide composition comprises:
a sum of rhamnose, galacturonic acid, arabinose, fucose, and mannose subunits in an amount of at least 5 wt. %, based on total weight of saccharide subunits; and
a sum of arabinose subunits in an amount of 20 wt. % to 90 wt. %, based on total weight of saccharide subunits; and a sum of galactose subunits in an amount of 5 wt. % to 60 wt. %, based on total weight of saccharide subunits.

24. The method of claim 22, wherein at least 70 wt. % of the non-terminal glucose subunits are β1,3 linked, β1,4 linked, and/or β1,6 linked.

25.-28. (canceled)

29. The method of claim 22, wherein the oligosaccharide composition comprises:
a sum of mannose subunits in an amount of at least 50 wt. %, based on total weight of saccharide subunits; and a sum of galactose subunits in an amount of less than 20 wt. %, based on total weight of saccharide subunits; and
a sum of arabinose subunits in an amount of 0 wt. % to 50 wt. %, based on total weight of saccharide subunits.

30. The method of claim 22, wherein the oligosaccharide composition comprises:
non-terminal xylose subunits, wherein at least 70 wt. % of the non-terminal xylose subunits have at least one 4-linkage; and
arabinose subunits, wherein at least 70 wt. % of the arabinose subunits are terminal arabinose subunits.

31. (canceled)

32. The method of claim 22, wherein the oligosaccharide composition is derived from a material comprising:
beechwood, larch, potato, carob, rye, wheat, corn, Sterculia, Saccharomyces Cerevisiae, Astragalus, pea, Xanthamonas campestris, coffee, sphingomonas elodea, soy, carrot, sugarcane, kelp, sea lettuce, olive, or any combination thereof; or
xylan, arabinogalactan, galactan, galactomannan, arabinoxylan, karaya gum, yeast mannan, yeast beta glucan, tragacanth gum, arabinan, xantham gum, galactan, mannan, gellen gum, pectin, fucoidan, or any combination thereof.

33. (canceled)

34. The method of claim 22, wherein the oligosaccharide composition comprises CLX103, CLX105, CLX109, CLX111, CLX114, CLX107, CLX115a, CLX117, CLX122, CLX122-DS, CLX122-DSF, CLX123, CLX124, CLX125, CLX126, CLX127, CLX128, CLX129, CLX130, CLX131, or any combination thereof.

35. The method of claim 22, wherein the microbiota comprise:
Bacteroides thetaiotaomicron, Bacteroides ovatus, Bacteroides fragilis, Akkermansia Mucinophila, Bacteroides eggerthii, Ruminococcus bromii, Eubacterium dolichum, Veillonella parvula, or any combination thereof.

36. The method of claim 22, wherein the method modulates the microbiota to produce the at least one short chain fatty acid, wherein the at least one short chain fatty acid comprises propionate.

37. The method of claim 1, wherein the oligosaccharide composition comprises the at least one third feature.

38. The method of claim 37, wherein the oligosaccharide composition comprises:
(1) a sum of glucose, galactose, mannose subunits in an amount of at least 50 wt. %, based on total weight of saccharide subunits; and
a sum of arabinose subunits in an amount of less than 5 wt. %, based on total weight of saccharide subunits;
(2) less than 10 wt. % 2-linked mannose subunits, based on total weight of saccharide subunits;
(3) non-terminal glucose subunits, wherein at least 70 wt. % of the non-terminal glucose subunits have at least one 3-linkage and/or at least one 4-linkage: or
(4) non-terminal xylose subunits, wherein at least 70 wt. % the non-terminal xylose subunits have at least one 4-linkage; and arabinose subunits, wherein at least 70 wt. % of the arabinose subunits are terminal arabinose subunits; or
any combination thereof.

39.-44. (canceled)

45. The method of claim 37, wherein the oligosaccharide composition comprises non-terminal glucose subunits, wherein (1) 20 wt. % to 100 wt. % of the non-terminal glucose subunits have at least one 3-linkage, based on total weight of non-terminal saccharide subunits; and/or (2) 33 wt. % to 80 wt. % of the non-terminal glucose subunits have at least one 4-linkage, based on total weight of non-terminal saccharide subunits.

46.-47. (canceled)

48. The method of claim 37, wherein the oligosaccharide composition comprises a weight ratio of glucose subunits having at least one 4-linkage to glucose subunits having at least one 3-linkage of between 2:1 to 4:1.

49.-52. (canceled)

53. The method of claim 37, wherein the oligosaccharide composition is derived from a material comprising:
Alcaligenes faecalis, konjac, locust bean, Icelandic moss, carob, barley, oat, sugar cane, or any combination thereof; or
curdlan glucomannan, galactomannan, lichenin, cereal beta glucan, sugar cane, or any combination thereof.

54. (canceled)

55. The method of claim 37, wherein the oligosaccharide composition comprises CLX101, CLX102, CLX108, CLX110, CLX111, CLX112, CLX115, CLX115-AL, CLX115-FC, CLX128, or any combination thereof.

56. The method of claim 37, wherein the microbiota comprise Clostridium cluster I, optionally selected from Clostridium butyricum.

57. The method of claim 37, wherein the method modulates the microbiota to produce the at least one short chain fatty acid, wherein the at least one short chain fatty acid comprises betahydroxybutyrate.

58.-59. (canceled)

60. The method of claim 1, wherein the at least one short chain fatty acid comprises butyrate, propionate, betahydroxybutyrate, lactate, acetate, or any combination thereof.

61.-81. (canceled)

82. A method for modulating microbiota to: produce at least one indole derivative, metabolize bile acid and/or bile salt, increase an abundance of the microbiota, or any combination thereof; the method comprising:
contacting the microbiota with a formulation comprising an oligosaccharide composition;
wherein the method modulates the microbiota to: produce the at least one indole derivative, metabolize the bile acids and/or bile salts, increase the abundance of the microbiota, or any combination thereof; and
wherein the oligosaccharide composition comprises:
a sum of galactose subunits in an amount of 3 wt. % to 60 wt. % galactose, based on total weight of saccharide subunits; and a sum of arabinose subunits in an amount of 20 wt. % to 90 wt. %, based on total weight of saccharide subunits;
non-terminal glucose subunits, wherein (1) 20 wt. % to 100 wt. % of the non-terminal glucose subunits have at least one 3-linkage, based on total weight of non-terminal saccharide subunits; and/or (2) 33 wt. % to 80 wt. % of the non-terminal glucose subunits have at least one 4-linkage, based on total weight of non-terminal saccharide subunits;
a sum of mannose subunits in an amount of 33 wt. % to 70 wt. %, based on total weight of saccharide subunits; and a sum of glucose subunits in an amount of 18 wt. % to 45 wt. %, based on total weight of saccharide subunits;
a sum of xylose subunits in an amount of 33 wt. % to 70 wt. %, based on total weight of saccharide subunits; and a sum of arabinose subunits in an amount of 18 wt. % to 45 wt. %, based on total weight of saccharide subunits; or
any combination thereof.

83.-98. (canceled)

99. The method of claim 82, wherein the oligosaccharide composition is derived from a material comprising:
konjac, wheat, corn, rye, oat, barley, pea, carrot, olive, or any combination thereof; or
glucomannan, arabinoxylan, cereal beta glucan, arabinan, arabinogalactan, pectin, or any combination thereof.

100. (canceled)

101. The method of claim 82, wherein the oligosaccharide composition comprises CLX102, CLX114, CLX115, CLX115-AL, CLX115-FC, CLX122-DS, CLX122-DSF, CLX122, CLX127, CLX131, or any combination thereof.

102.-103. (canceled)

104. The method of claim 82, wherein the at least one indole derivative comprises indole-3-aldehyde, indole-3-lactate, indole-3-propionate, or any combination thereof.

105. (canceled)

106. The method of claim 82, wherein the microbiota comprise:
Bifidobacterium, optionally selected from Bifidobacterium longum, Bifidobacterium pseudocatenulatum, Bifidobacterium lactis, Bifidobacterium infantus, Bifidobacterium adolescentis, or Bifidobacterium animalis, or any combination thereof;
Lactobacilli, optionally selected from Lactobacillus lactis, Lactobacillus rhamnosus, or Lactobacillus plantarum, or any combination thereof;
Clostridium sporongenes or Peptosreptococcus, or a combination thereof; or
any combination thereof.

107. (canceled)

108. The method of claim 82, wherein the bile acids and/or bile salts comprise cholate, deoxycholate, lithocholate, chenodeoxycholate, ursodeoxycholate, 7-oxolithocholate, 7-oxo-deoxycholate, or any combination thereof.

109.-127. (canceled)

128. An oligosaccharide composition, comprising:
a sum of galactose and arabinose subunits in an amount of at least 33 wt. %, based on total weight of saccharide subunits;
a sum of 3-linked and 4-linked glucose subunits in an amount of between 7-50% wt. %, based on total weight of saccharide subunits;
at least 35 wt. % fucose subunits, based on total weight of saccharide subunits; or
at least 25 wt. % rhamnose subunits, based on total weight of saccharide subunits;
or any combination thereof.

129.-151. (canceled)

152. The oligosaccharide composition of claim 128, wherein the oligosaccharide composition is CLX116, CLX117, CLX119, CLX122, CLX122-DS, CLX122-DSF, CLX124, CLX126, CLX127, CLX131, CLX132, CLX133, or any combination thereof; or
CLX123, CLX125, CLX128, CLX130, or any combination thereof: or
CLX129; or
CLX107.

153.-212. (canceled)

213. A formulation comprising the oligosaccharide composition of claim 128.

214.-222. (canceled)
